(19) United States
(12) Patent Application Publication
(10) Pub. No.: US 2026/0117176 A1
(43) Pub. Date: Apr. 30, 2026

(54) MODIFIED IMMUNE EFFECTOR CELLS WITH IMPROVED EFFICACY

(71) Applicant: Beam Therapeutics Inc., Cambridge, MA (US)

(72) Inventors: Leila Peraro, Cambridge, MA (US); Sami Kebede, Cambridge, MA (US)

(73) Assignee: Beam Therapeutics Inc., Cambridge, MA (US)

(21) Appl. No.: 19/354,669

(22) Filed: Oct. 9, 2025

Related U.S. Application Data
(63) Continuation of application No. PCT/US2024/018668, filed on Mar. 6, 2024.
(60) Provisional application No. 63/495,682, filed on Apr. 12, 2023.

(51) Int. Cl. C12N 5/0783; A61K 40/11; A61K 40/31; A61K 40/42; A61P 35/00; C07K 16/28; C12N 9/78; C12N 15/11

(52) U.S. Cl. CPC C12N 5/0636; A61K 40/11; A61K 40/31; A61K 40/4211; A61K 40/4212; A61P 35/00; C07K 16/2803; C12N 9/78; C12N 15/11; C12Y 305/04004; C07K 2317/622; C07K 2319/02; C12N 2310/20; C12N 2510/00

(57) ABSTRACT

Multiplex base edited chimeric antigen receptor (CAR)-expressing immune effector cells (e.g., T or NK cells) having increased resistance to development of an exhausted phenotype (e.g., increased cytotoxicity, proliferation, survival, and/or cytokine production) after repeated or continuous stimulation by an antigen relative to unedited CAR immune effector cells, compositions containing the cells, methods for the preparation of the cells, and methods for use of the cells in treating a disease or disorder (e.g., an autoimmune disorder or a neoplasia, such as a leukemia).

### CROSS REFERENCE TO RELATED APPLICATIONS

This application is a continuation under 35 U.S.C. § 111(a) of PCT International Patent Application No. PCT/US2024/018668, filed Mar. 6, 2024, designating the United States and published in English, which claims priority to and the benefit of U.S. Provisional Application No. 63/495,682, filed Apr. 12, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Mar. 6, 2024, is named 180802-055402_PCT_SL.xml and is 2,772,339 bytes in size.

### BACKGROUND OF THE DISCLOSURE

Autologous and allogeneic immunotherapies are approaches for treating a disease or disorder (e.g., a neoplasia or an autoimmune disease) in which immune cells (e.g., T cells) expressing chimeric antigen receptors are administered to a subject. To generate an immune cell that expresses a chimeric antigen receptor (CAR), the immune cell is first collected from the subject (autologous) or a donor separate from the subject receiving treatment (allogeneic) and genetically modified to express the chimeric antigen receptor. The resulting cell expresses the chimeric antigen receptor on its cell surface (e.g., CAR-T cell), and upon administration to the subject, the chimeric antigen receptor binds to the antigen expressed by a cell associated with the disease or disorder. This interaction with the antigen activates the CAR-expressing immune cell (e.g., a CAR-T cell), which then kills, inactivates, or neutralizes cells or molecules associated with the disease or disorder. But for autologous or allogeneic cell therapy to be effective and efficient, significant conditions and cellular responses, such as immune cell signaling inhibition or immune cell exhaustion, must be overcome or avoided. Autologous cell therapies have a number of disadvantages, including long manufacturing times and the requirement that the patient cells are suitable despite previous therapies or disease state. Challenges are also associated with allogeneic cell therapy, including graft-versus-host disease (GVHD), and host rejection of CAR-T cells. Currently, CAR-expressing immune effector cells develop an exhausted phenotype (e.g., reduced cytotoxicity) when repeatedly or continuously contacted with a target antigen. Thus, there is a significant need for techniques for improving immune effector cell resistance to exhaustion associated with continuous or repeated exposure to a target antigen in vivo.

### SUMMARY OF THE DISCLOSURE

As described below, the present disclosure features multiplex base edited chimeric antigen receptor (CAR)-expressing immune effector cells (e.g., T or NK cells) having increased resistance to development of an exhausted phenotype (e.g., increased cytotoxicity, proliferation, survival, and/or cytokine production) after repeated or continuous stimulation by an antigen relative to unedited CAR immune effector cells, compositions containing the cells, methods for the preparation of the cells, and methods for use of the cells in treating a disease or disorder (e.g., an autoimmune disorder or a neoplasia, such as a leukemia).

In one aspect, the disclosure features a method for producing a modified immune effector cell having reduced exhaustion after antigen exposure relative to an unedited immune effector cell. The method involves contacting the cell with (i) a base editor, or a polynucleotide encoding the base editor. The base editor contains a programmable DNA binding domain and a deaminase domain. The method further involves contacting the cell with (ii) a guide polynucleotide, or a polynucleotide encoding the guide polynucleotide. The guide polynucleotide directs the base editor to effect a nucleobase alteration in a polynucleotide encoding a polypeptide selected from one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The method also involves contacting the cell with (iii) a guide polynucleotide, or a polynucleotide encoding the guide polynucleotide.

The guide polynucleotide directs the base editor to effect a nucleobase alteration in a polynucleotide encoding a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC). Each nucleobase alteration effects a reduction in expression of the encoded polypeptide, thereby producing the modified immune effector cell.

In another aspect, the disclosure features a modified immune cell having reduced exhaustion after antigen exposure relative to an unedited immune effector cell. The modified immune cell contains a nucleobase alteration that reduces or eliminates expression of (i) a polypeptide selected from one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The nucleobase alteration also reduces or eliminates expression of (ii) a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In another aspect, the disclosure features a modified immune effector cell having reduced exhaustion after antigen exposure relative to an unedited immune effector cell. The modified immune effector cell expresses a chimeric antigen receptor targeting an antigen associated with a disease or disorder. The modified immune effector cell has reduced or undetectable expression of (i) one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The modified immune cell also has reduced or undetectable expression of (ii) one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In another aspect, the disclosure features a modified immune cell having increased efficacy relative to an unedited immune effector cell produced according to the method of any aspect of the disclosure delimited herein, or embodiments thereof.

In another aspect, the disclosure features a base editor system that contains (i) a base editor, or a polynucleotide encoding the base editor. The base editor contains a programmable DNA binding domain and a deaminase domain. The base editor system also contains (ii) a guide polynucleotide, or a polynucleotide encoding the guide polynucleotide. The guide polynucleotide directs the base editor to effect a nucleobase alteration in a polynucleotide encoding a polypeptide selected from one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The base editor system further contains (iii) a guide polynucleotide, or a polynucleotide encoding the guide polynucleotide. The guide polynucleotide directs the base editor to effect a nucleobase alteration in a polynucleotide encoding a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In another aspect, the disclosure features a cell containing the base editor system of any aspect of the disclosure delimited herein, or embodiments thereof.

In another embodiment, the disclosure features a vector or set of vectors containing (i) a polynucleotide encoding a base editor. The base editor contains a programmable DNA binding domain and a deaminase domain. The vector or set of vectors also contains (ii) a guide polynucleotide, or a polynucleotide encoding the guide polynucleotide. The guide polynucleotide directs the base editor to effect a nucleobase alteration in a polynucleotide encoding a polypeptide selected from one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The vector or set of vectors also contains (iii) a guide polynucleotide, or a polynucleotide encoding the guide polynucleotide. The guide polynucleotide directs the base editor to effect a nucleobase alteration in a polynucleotide encoding a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In another aspect, the disclosure features a pharmaceutical composition containing an effective amount of the modified immune cell, the base editor system, the cell, or the vector of any aspect of the disclosure delimited herein, or embodiments thereof, and a pharmaceutically acceptable excipient.

In another aspect, the disclosure features a pharmaceutical composition containing (i) a base editor, or a polynucleotide encoding the base editor. The base editor contains a programmable DNA binding domain and a deaminase domain. The pharmaceutical composition also contains (ii) a guide polynucleotide, or a polynucleotide encoding the guide polynucleotide. The guide polynucleotide directs the base editor to effect a nucleobase alteration in a polynucleotide encoding a polypeptide selected from one or more of ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. The pharmaceutical composition also contains (iii) a guide polynucleotide, or a polynucleotide encoding the guide polynucleotide. The guide polynucleotide directs the base editor to effect a nucleobase alteration in a polynucleotide encoding a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In another aspect, the disclosure features a method of treating a disease or disorder in a subject. The method involves administering to the subject an effective amount of a modified immune cell, the cell, or the pharmaceutical composition of any aspect of the disclosure delimited herein, or embodiments thereof.

In another aspect, the disclosure features a kit containing the modified immune cell, the cell, the pharmaceutical composition, or the composition of any aspect of the disclosure delimited herein, or embodiments thereof, for use in the method of any aspect of the disclosure delimited herein, or embodiments thereof.

In another aspect, the disclosure features a modified immune cell having reduced exhaustion after antigen exposure relative to an unedited immune effector cell, where the modified immune cell is produced according to the method of any aspect of the disclosure, or embodiments thereof.

In another aspect, the disclosure features a method for producing a modified immune effector cell having reduced exhaustion after antigen exposure relative to an unedited immune effector cell. The method involves contacting the cell with (i) a base editor, or a polynucleotide encoding the base editor. The base editor contains a programmable DNA binding domain and a deaminase domain. The method further involves contacting the cell with (ii) a guide polynucleotide, or a polynucleotide encoding the guide polynucleotide. The the guide polynucleotide directs the base editor to effect a nucleobase alteration in a polynucleotide encoding a Roquin-1 polypeptide. The method also involves contacting the cell with (iii) a guide polynucleotide, or a polynucleotide encoding the guide polynucleotide. The guide polynucleotide directs the base editor to effect a nucleobase alteration in a polynucleotide encoding a polypeptide selected from one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC). Each nucleobase alteration effects a reduction in expression of the encoded polypeptide, thereby producing the modified immune effector cell.

In another aspect, the present disclosure features a method for reducing or eliminating the presence of a neoplasia in a subject. The method involves administering to the subject a cell produced according to the method of any aspect of the disclosure, or embodiments thereof, where the cell expresses a chimeric antigen receptor targeting an antigen associated with the neoplasia.

In any aspect of the disclosure delimited herein, or embodiments thereof, the method further involves expressing one or two chimeric antigen receptors in the cell.

In any aspect of the disclosure delimited herein, or embodiments thereof, the method further involves contacting the cell with a guide polynucleotide, or a polynucleotide encoding the guide polynucleotide. The guide polynucleotide directs the base editor to effect a nucleobase alteration in a polynucleotide encoding an antigen(s) bound by the chimeric antigen receptor(s). The nucleobase alteration effects a reduction in expression of the antigen.

In any aspect of the disclosure delimited herein, or embodiments thereof, the base editing reduces expression of a CISH, CBLB, SOCS1, Roquin-1, DNMT3A, FLI-1, RASA2, Regnase-1, CD5, or PDP1B polypeptide in the cell. In any aspect of the disclosure delimited herein, or embodiments thereof, the base editing reduces expression of a TRAC, B2M, and CIITA polypeptide in the cell. In any aspect of the disclosure delimited herein, or embodiments thereof, the base editing reduces expression of a CD3g, B2M, and CIITA polypeptide in the cell. In any aspect of the disclosure delimited herein, or embodiments thereof, the base editing reduces expression of PD1 in the cell.

In any aspect of the disclosure delimited herein, or embodiments thereof, the guide polynucleotide of (ii) is selected from one or more of EF1, EF2, EF3, EF4, EF5, EF6, EF7, EF8, EF9, EF10, EF11, EF12, EF13, EF14, EF15, EF16, EF17, EF18, EF19, EF20, EF21, EF22, EF23, EF24, EF25, EF26, EF27, EF28, EF29, EF46, EF47, EF48, EF49, EF50, EF51, EF52, EF53, EF54, EF55, EF56, EF57, EF58, EF59, EF60, EF61, EF62, EF63, EF64, EF65, EF66, EF67, EF68, EF69, EF70, EF71, EF72, EF73, EF74, EF75, EF76, EF77, EF85, EF86, EF87, EF88, EF89, EF90, EF91, EF92, EF93, EF94, EF95, EF96, EF97, EF98, EF99, EF100, EF101, EF102, EF103, EF104, EF105, EF106, EF107, EF108, EF109, EF110, EF111, EF112, EF113, EF114, EF115, EF116, EF117, EF118, EF119, and EF120. In any aspect of the disclosure delimited herein, or embodiments thereof, the guide polynucleotide of (ii) is selected from one or more of EF1, EF2, EF3, EF4, EF7, EF8, EF14, EF15, EF20, EF24, EF25, EF46, EF47, EF48, EF49, EF50, EF51, EF52, EF54, EF55, EF56, EF57, EF58, EF59, EF60, EF61, EF62, EF63, EF64, EF65, EF66, EF67, EF70, EF74, EF76, EF85, EF86, EF91, EF94, EF95, EF96, EF97, EF98, EF99, EF100, EF101, EF102, EF103, EF104, EF105, EF106, EF107, EF108, EF109, EF110, EF111, EF112, EF113, EF114, and EF117. In any aspect of the disclosure delimited herein, or embodiments thereof, the guide polynucleotide of (ii) is selected from one or more of EF1, EF2, EF4, EF7, EF8, EF15, EF20, EF25, EF96, EF94.

In any aspect of the disclosure delimited herein, or embodiments thereof, the nucleobase alterations effect an alteration in the ratio of CD4+ to CD8+ cells in a population of the modified immune effector cells following one or more exposures to antigen relative to a population of unedited immune cells. In any aspect of the disclosure delimited herein, or embodiments thereof, the nucleobase alterations effect an increase in the proportion of CD4+ cells in the population of the modified immune effector cells following one or more exposures to antigen relative to a population of unedited immune cells. In any aspect of the disclosure delimited herein, or embodiments thereof, the nucleobase alterations effect an increase in CD25, ICOS, OX40, and/or CD28 expression in the modified immune effector cell following one or more exposures to antigen relative to an unedited immune cell. In any aspect of the disclosure delimited herein, or embodiments thereof, the nucleobase alterations effect an increase in cytokine secretion and/or intracellular levels in the modified immune effector cell following one or more exposures to antigen relative to an unedited immune cell. In embodiments, the cytokine is selected from one or more of cytokines granzyme B (GZMB), interferon gamma (IFNg), interleukin-2 (IL-2), and tumor necrosis factor alpha (TNFa). In any aspect of the disclosure delimited herein, or embodiments thereof, the nucleobase alterations effect an increase in EOMES+ and/or T-bet− cells in a population of the modified immune effector cells following one or more exposures to antigen relative to a population of unedited immune cells. In any aspect of the disclosure delimited herein, or embodiments thereof, the nucleobase alterations effect an increase in marker of proliferation KI67 expression in the modified immune effector cell following one or more exposures to antigen relative to an unedited immune cell. In any aspect of the disclosure delimited herein, or embodiments thereof, the base editing reduces expression of a DCK, DGKα, DGKζ, EIF2A, RASA2, PFN1, or ARID1A polypeptide in the cell, thereby increasing immune cell proliferation following one or more exposures to antigen relative to an unedited immune cell. In any aspect of the disclosure delimited herein, or embodiments thereof, the base editing reduces expression of a Roquin-1 polypeptide in the cell, thereby increasing antigen-dependent proliferation following one or more exposures to antigen relative to an unedited immune cell. In any aspect of the disclosure delimited herein, or embodiments thereof, the base editing reduces expression of a CISH, CBLB, PTP1B, SOCS1, Roquin-1, DNMT3A, FLI1, Chop, or Regnase-1 polypeptide in the cell, thereby increasing cytotoxic capacity following one or more exposures to antigen relative to an unedited immune cell. In any aspect of the disclosure delimited herein, or embodiments thereof, the base editing reduces expression of a DCK, CD5, DGKα, DGKζ, DHX37, EIF2A, ID3, IKZF2, IL-6, PFN1, PRDM1, PRKACA, PTPN6, RASA2, SOX4, TLE, TMEM184B, or TMEM222 polypeptide in the cell, thereby increasing cytotoxic capacity following one or more exposures to antigen relative to an unedited immune cell. In any aspect of the disclosure delimited herein, or embodiments thereof, the base editing reduces expression of a CISH, FLI-1, SOCS1, or Roquin-1 polypeptide in the cell, thereby increasing cytotoxic capacity following one or more exposures to antigen relative to an unedited immune cell. In any aspect of the disclosure delimited herein, or embodiments thereof, the base editing reduces expression of a CISH, CBLB, SOCS1, Roquin-1, or DNMT3A polypeptide in the cell, thereby increasing KI67 polypeptide expression, reducing T-bet polypeptide expression, and/or increasing EOMES expression in the cell following one or more exposures to antigen relative to an unedited immune cell. In any aspect of the disclosure delimited herein, or embodiments thereof, the base editing reduces expression of a CISH, SOCS1, or Roquin-1 polypeptide in the cell, thereby increasing secretion of granzyme B (GZMB), interferon gamma (IFNg), interleukin-2 (IL-2), or tumor necrosis factor alpha (TNFa) following one or more exposures to antigen relative to an unedited immune cell. In any aspect of the disclosure delimited herein, or embodiments thereof, the one or more exposures to antigen is, or is at least 1, 2, 3, 4, 5, 6, 7, 8, 9, or 10 exposures.

In any aspect of the disclosure delimited herein, or embodiments thereof, the immune cell is an NK cell or T cell.

In any aspect of the disclosure delimited herein, or embodiments thereof, the base editing efficiency for the nucleobase alterations is at least about 80%.

In any aspect of the disclosure delimited herein, or embodiments thereof, the immune cell further contains one or two chimeric antigen receptor targeting one or two antigen associated with a disease or disorder. In any aspect of the disclosure delimited herein, or embodiments thereof, the modified immune cell contains reduced or undetectable expression of an antigen(s) bound by the chimeric antigen receptor(s).

In any aspect of the disclosure delimited herein, or embodiments thereof, the disease is a neoplasia. In embodiments, the neoplasia is a lymphoma. In embodiments, the lymphoma is a mantle cell lymphoma or a B cell lymphoma.

In any aspect of the disclosure delimited herein, or embodiments thereof, the modified immune cell has reduced or undetectable levels of expression of a CISH, CBLB, SOCS1, Roquin-1, DNMT3A, FLI-1, RASA2, Regnase-1, CD5, or PDP1B polypeptide in the cell. In any aspect of the disclosure delimited herein, or embodiments thereof, the immune cell has reduced or undetectable levels of expression of a TRAC, B2M, and CIITA polypeptide. In any aspect of the disclosure delimited herein, or embodiments thereof, the modified immune cell has reduced or undetectable levels of expression of a CD3g, B2M, and CIITA polypeptide. In any aspect of the disclosure delimited herein, or embodiments thereof, the modified immune cell has reduced or undetectable levels of expression of PD1. In any aspect of the disclosure delimited herein, or embodiments thereof, a population of the modified immune cells has an alteration in the ratio of CD4+ to CD8+ cells following one or more exposures to antigen relative to a population of unedited immune cells. In any aspect of the disclosure delimited herein, or embodiments thereof, a population of the modified immune cells has an increase in the proportion of CD4+ cells following one or more exposures to antigen relative to a population of unedited immune cells. In any aspect of the disclosure delimited herein, or embodiments thereof, the modified immune cell has an increase in CD25, ICOS, OX40, and/or CD28 expression following one or more exposures to antigen relative to an unedited immune cell. In any aspect of the disclosure delimited herein, or embodiments thereof, the modified immune cell has an increase in cytokine secretion and/or intracellular levels following one or more exposures to antigen relative to an unedited immune cell. In embodiments, the cytokine is selected from one or more of cytokines granzyme B (GZMB), interferon gamma (IFNg), interleukin-2 (IL-2), and tumor necrosis factor alpha (TNFa). In any aspect of the disclosure delimited herein, or embodiments thereof, a population of the modified immune cells has an increase in EOMES+ and/or T-bet− cells following one or more exposures to antigen relative to a population of unedited immune cells. In any aspect of the disclosure delimited herein, or embodiments thereof, the modified immune cell has an increase in marker of proliferation KI67 expression following one or more exposures to antigen relative to an unedited immune cell. In any aspect of the disclosure delimited herein, or embodiments thereof, the immune cell has reduced expression of a DCK, DGKα, DGKζ, EIF2A, RASA2, PFN1, or ARID1A polypeptide, and has increased immune cell proliferation following one or more exposures to antigen relative to an unedited immune cell. In any aspect of the disclosure delimited herein, or embodiments thereof, the immune cell has reduced expression of Roquin-1 polypeptide and has increased antigen-dependent proliferation following one or more exposures to antigen relative to an unedited immune cell. In any aspect of the disclosure delimited herein, or embodiments thereof, the immune cell has reduced expression of a CISH, CBLB, PTP1B, SOCS1, Roquin-1, DNMT3A, FLI1, Chop, or Regnase-1 polypeptide, and has increased cytotoxic capacity following one or more exposures to antigen relative to an unedited immune cell. In any aspect of the disclosure delimited herein, or embodiments thereof, the immune cell has reduced expression of a DCK, CD5, DGKa, DGKz, DHX37, EIF2A, ID3, IKZF2, IL-6, PFN1, PRDM1, PRKACA, PTPN6, RASA2, SOX4, TLE, TMEM184B, or TMEM222 polypeptide, and has increased cytotoxic capacity following one or more exposures to antigen relative to an unedited immune cell. In any aspect of the disclosure delimited herein, or embodiments thereof, the immune cell has reduced expression of a CISH, FLI-1, SOCS1, or Roquin-1 polypeptide in the cell, and has increased cytotoxic capacity following one or more exposures to antigen relative to an unedited immune cell. In any aspect of the disclosure delimited herein, or embodiments thereof, the immune cell has reduced expression of a CISH, CBLB, SOCS1, Roquin-1, or DNMT3A polypeptide in the cell, and has increased KI67 polypeptide expression, reducing T-bet polypeptide expression, and/or increasing EOMES expression in the cell following one or more exposures to antigen relative to an unedited immune cell. In any aspect of the disclosure delimited herein, or embodiments thereof, the immune cell has reduced expression of a CISH, SOCS1, or Roquin-1 polypeptide in the cell, and has increased secretion of granzyme B (GZMB), interferon gamma (IFNg), interleukin-2 (IL-2), or tumor necrosis factor alpha (TNFa) following one or more exposures to antigen relative to an unedited immune cell.

In any aspect of the disclosure delimited herein, or embodiments thereof, the guide polynucleotide of (ii) contains a polynucleotide sequence with at least about 85% sequence identity to a sequence selected from those listed in Table 1 or Table 2A, or a variant thereof having an extension or truncation 1, 2, 3, 4, or 5 nucleotides in length at the 3′ and/or 5′ end. In any aspect of the disclosure delimited herein, or embodiments thereof, the guide polynucleotide of (ii) contains a polynucleotide sequence with at least about 85% sequence identity to a sequence selected from those listed in Table 1 or Table 2A, or a variant thereof having an extension or truncation 1, 2, 3, 4, or 5 nucleotides in length at the 3′ and/or 5′ end.

In any aspect of the disclosure delimited herein, or embodiments thereof, the vector or set of vectors contains a lentiviral vector or adeno-associated viral vector.

In any aspect provided herein, or embodiments thereof, the method is not a process for modifying the germline genetic identity of human beings.

In any aspect provided herein, or embodiments thereof, the base editing reduces expression of a Roquin-1, DGKζ, or FLI-1 polypeptide in the cell. In any aspect provided herein, or embodiments thereof, the modified immune cell has reduced or undetectable levels of expression of a Roquin-1, DGKζ, or FLI-1 polypeptide in the cell.

In any aspect provided herein, or embodiments thereof, the guide polynucleotide contains a spacer containing, the following nucleotide sequence AUGUACCUGGUCCAAGGAAC (SEQ ID NO: 456). In any aspect provided herein, or embodiments thereof, the guide polynucleotide contains the following nucleotide sequence:

(SEQ ID NO: 427)
AUGUACCUGGUCCAAGGAACGUUUUAGAGCUAGAAAUAGCAAGUU
AAAAUAAGGCUAGUCCGUUAUCAACUUGAAAAAGUGGCACCGAGU
CGGUGCUUUU.

In any aspect provided herein, or embodiments thereof, the base editing reduces expression of the Roquin-1 polypeptide in the cell. In any aspect provided herein, or embodiments thereof, the method is associated with improved suppression of tumor recurrence in the subject relative to a subject administered an unedited immune cell expressing the chimeric antigen receptor. In any aspect provided herein, or embodiments thereof, the cells have increased maintenance of a central memory phenotype relative to unedited immune cells administered to a subject and expressing the chimeric antigen receptor.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “AT-rich interaction domain-containing protein 1A (ARID1A) polypeptide” is meant a protein having at least about 85% amino acid sequence identity to GenBank Accession No. BAA23269.1, which is provided below, or a fragment thereof having immunomodulatory activity.

>BAA23269.1 B120 [Homo sapiens]
(SEQ ID NO: 826)
MDQMGKMRPQPYGGTNPYSQQQGPPSDPQQGHGYPGQPYGSQTPQ
RYPMTVQGRAQSAMGGLSYTQQIPPYGQQGPSGYGQQGQTPYYNQ
QSPHPQQQQPPYSQQPPSQTPHAQPSYQQQPQSQPPQLQSSQPPY
SQQPSQPPHQQSPAPYPSQQSTTQQHPQSQPPYSQPQAQSPYQQQ
QPQQPAPSTLSQQAAYPQPQSQQSQQTAYSQQRFPPPQELSQDSF
GSQASSAPSMTSSKGGQEDMNLSLQSRPSSLPDLSGSIDDLPMGT
EGALSPGVSTSGISSSQGEQSNPAQSPFSPHTSPHLPGIRGPSPS
PVGSPASVAQSRSGPLSPAAVPGNQMPPRPPSGSSDSIMHPSMNQ
SSIAQDRGYMQRNSQMPQYSSPQPGSALSPRQLSGGQIHTGMGSY
QQNSMGSYGPQGGQYGPQGGYPRQPNYNALPNANYPSAGMAGGIN
PMGAGGQMHGQPGIPPYGTLPPGRMSHASMGNRPYGPNNGQYATS
GWVRDVSPPGGMNRKTQETAVAMHVAANSIQNRPPGYPNMNQGGM
MGTGPPYGQGINSMAGMINPQGPPYSMGGTMANNSAGMAASPEMM
GLGDVKLTPATKMNNKADGTPKTESKSKKSSSSTTTNEKITKLYE
LGGGPERKMWVDRYLAFTEEKAMGMTNLPAVGRKPLDLYRLYVSV
KEIGGLTQVNKNKKWRELATNLNVGTSSSAASSLKKQYIQCLYAF
ECKIERGEDPPPDIFAAADSKKSQPKIQPPSPAGSGSMQGPQTPQ
STSSSMAEGGDLKPPTPASTPHSQIPPLPGMSRSNSVGIQDAFND
GSDSTFQKRNSMTPNPGYQPSMNTSDMMGRMSYEPNKDPYGSMRK
APGSDPFMSSGQGPNGGMGDPYSRAAGPGLGNVAMGPRQHYPYGG
PYDRVRTEPGIGPEGNMSTGAPQSNLMPSNPDSGMYSPSRYPPQQ
QQQQQQRHDSYGNQFSTQGTPSGSPFPSQQTTMYQQQQQNYKRPM
DGTYGPPAKRHEGEMYSVPYSTGQGLPQQQQLPPAQPQPASQPQA
AQPSPQQDVYNQYGNAYPATATAATERRPAGGPQNQFPFQFGRDR
VSAPPGTNAQQNMPPQMMGGPIQASAEVAQQGTMWQGRNDMTYNY
ANRQSTALPPRAPPIMA.

By “AT-rich interaction domain-containing protein 1A (ARID1A) polynucleotide” is meant a polynucleotide encoding a ARID1A polypeptide, as well as the introns, exons, 3′ untranslated regions, 5′ untranslated regions, and regulatory sequences associated with its expression, or fragments thereof. In embodiments, a ARID1A polynucleotide is the genomic sequence, cDNA, mRNA, or gene associated with and/or required for ARID1A expression. An exemplary ARID1A nucleic acid sequence is provided below (GenBank Accession No. AB001895.1:288-3716).

>AB001895.1:288-3716 Homo sapiens mRNA for B120, Complete Cds
ATGGATCAGATGGGCAAGATGAGACCTCAGCCATATGGCGGGACTAACCCATACTCGCAGCA ACAGGGACCTCCGTCAGACCCGCAGCAAGGACATGGGTACCCAGGGCAGCCATACGGGTCCC AGACCCCGCAGCGGTACCCGATGACCGTGCAGGGCCGGGCGCAGAGTGCCATGGGCGGCCTC TCTTATACACAGCAGATTCCTCCTTATGGACAACAAGGCCCCAGCGGGTATGGTCAACAGGG CCAGACTCCATATTACAACCAGCAAAGTCCTCACCCTCAGCAGCAGCAGCCACCCTACTCCC AGCAACCACCGTCCCAGACCCCTCATGCCCAACCTTCGTATCAGCAGCAGCCACAGTCTCAA CCACCACAGCTCCAGTCCTCTCAGCCTCCATACTCCCAGCAGCCATCCCAGCCTCCACATCA GCAGTCCCCGGCTCCATACCCCTCCCAGCAGTCGACGACACAGCAGCACCCCCAGAGCCAGC CCCCCTACTCACAGCCACAGGCTCAGTCTCCTTACCAGCAGCAGCAACCTCAGCAGCCAGCA CCCTCGACGCTCTCCCAGCAGGCTGCGTATCCTCAGCCCCAGTCTCAGCAGTCCCAGCAAAC TGCCTATTCCCAGCAGCGCTTCCCTCCACCGCAGGAGCTATCTCAAGATTCATTTGGGTCTC AGGCATCCTCAGCCCCCTCAATGACCTCCAGTAAGGGAGGGCAAGAAGATATGAACCTGAGC CTTCAGTCAAGACCCTCCAGCTTGCCTGATCTATCTGGTTCAATAGATGACCTCCCCATGGG GACAGAAGGAGCTCTGAGTCCTGGAGTGAGCACATCAGGGATTTCCAGCAGCCAAGGAGAGC AGAGTAATCCAGCTCAGTCTCCTTTCTCTCCTCATACCTCCCCTCACCTGCCTGGCATCCGA GGCCCTTCCCCGTCCCCTGTTGGCTCTCCCGCCAGTGTTGCTCAGTCTCGCTCAGGACCACT CTCGCCTGCTGCAGTGCCAGGCAACCAGATGCCACCTCGGCCACCCAGTGGCAGTTCGGACA GCATCATGCATCCTTCCATGAACCAATCAAGCATTGCCCAAGATCGAGGTTATATGCAGAGG AACTCCCAGATGCCCCAGTACAGTTCCCCCCAGCCCGGCTCAGCCTTATCTCCGCGTCAGCT TTCCGGAGGACAGATACACACAGGCATGGGCTCCTACCAGCAGAACTCCATGGGGAGCTATG GTCCCCAGGGGGGTCAGTATGGCCCACAAGGTGGCTACCCCAGGCAGCCAAACTATAATGCC TTGCCCAATGCCAACTACCCCAGTGCAGGCATGGCTGGAGGCATAAACCCCATGGGTGCCGG AGGTCAAATGCATGGACAGCCTGGCATCCCACCTTATGGCACACTCCCTCCAGGGAGGATGA GTCACGCCTCCATGGGCAACCGGCCTTATGGCCCTAACAATGGCCAATATGCCACCTCAGGT TGGGTCAGGGATGTGTCCCCACCAGGGGGCATGAACCGGAAAACCCAAGAAACTGCTGTCGC CATGCATGTTGCTGCCAACTCTATCCAAAACAGGCCGCCAGGCTACCCCAATATGAATCAAG GGGGCATGATGGGAACTGGACCTCCTTATGGACAAGGGATTAATAGTATGGCTGGCATGATC AACCCTCAGGGACCCCCATATTCCATGGGTGGAACCATGGCCAACAATTCTGCAGGGATGGC AGCCAGCCCAGAGATGATGGGCCTTGGGGATGTAAAGTTAACTCCAGCCACCAAAATGAACA ACAAGGCAGATGGGACACCCAAGACAGAATCCAAATCCAAGAAATCCAGTTCTTCTACTACA ACCAATGAGAAGATCACCAAGTTGTATGAGCTGGGTGGTGGGCCTGAGAGGAAGATGTGGGT GGACCGTTATCTGGCCTTCACTGAGGAGAAGGCCATGGGCATGACAAATCTGCCTGCTGTGG GTAGGAAACCTCTGGACCTCTATCGCCTCTATGTGTCTGTGAAGGAGATTGGTGGATTGACT CAGGTCAACAAGAACAAAAAATGGCGGGAACTTGCAACCAACCTCAATGTGGGCACATCAAG CAGTGCTGCCAGCTCCTTGAAAAAGCAGTATATCCAGTGTCTCTATGCCTTTGAATGCAAGA TTGAACGGGGAGAAGACCCTCCCCCAGACATCTTTGCAGCTGCTGATTCCAAGAAGTCCCAG CCCAAGATCCAGCCTCCCTCTCCTGCGGGATCAGGATCTATGCAGGGGCCCCAGACTCCCCA GTCAACCAGCAGTTCCATGGCAGAAGGAGGAGACTTAAAGCCACCAACTCCAGCATCCACAC CACACAGTCAGATCCCCCCATTGCCAGGCATGAGCAGGAGCAATTCAGTTGGGATCCAGGAT GCCTTTAATGATGGAAGTGACTCCACATTCCAGAAGCGGAATTCCATGACTCCAAACCCTGG GTATCAGCCCAGTATGAATACCTCTGACATGATGGGGCGCATGTCCTATGAGCCAAATAAGG ATCCTTATGGCAGCATGAGGAAAGCTCCAGGGAGTGATCCCTTCATGTCCTCAGGGCAGGGC CCCAACGGCGGGATGGGTGACCCCTACAGTCGTGCTGCCGGCCCTGGGCTAGGAAATGTGGC GATGGGACCACGACAGCACTATCCCTATGGAGGTCCTTATGACAGAGTGAGGACGGAGCCTG GAATAGGGCCTGAGGGAAACATGAGCACTGGGGCCCCACAGTCGAATCTCATGCCTTCCAAC CCAGACTCGGGGATGTATTCTCCTAGCCGCTACCCCCCGCAGCAGCAGCAGCAGCAGCAGCA ACGACATGATTCCTATGGCAATCAGTTCTCCACCCAAGGCACCCCTTCTGGCAGCCCCTTCC CCAGCCAGCAGACTACAATGTATCAACAGCAACAGCAGAATTACAAGCGGCCAATGGATGGC ACATATGGCCCTCCTGCCAAGCGGCACGAAGGGGAGATGTACAGCGTGCCATACAGCACTGG GCAGGGGCTGCCTCAGCAGCAGCAGTTGCCCCCAGCCCAGCCCCAGCCTGCCAGCCAGCCAC AAGCTGCCCAGCCTTCCCCTCAGCAAGATGTATACAACCAGTATGGCAATGCCTATCCTGCC ACTGCCACAGCTGCTACTGAGCGCCGACCAGCAGGCGGCCCCCAGAACCAATTTCCATTCCA GTTTGGCCGAGACCGTGTCTCTGCACCCCCTGGCACCAATGCCCAGCAAAACATGCCACCAC AAATGATGGGCGGCCCCATACAGGCATCAGCTGAGGTTGCTCAGCAAGGCACCATGTGGCAG GGGCGTAATGACATGACCTATAATTATGCCAACAGGCAGAGCACGGCTCTGCCCCCCAGGGC CCCGCCTATCATGGCGTGA (SEQ ID NO: 827). An exemplary ARID1A gene sequence is provided at ENSEMBL Accession No. ENSG00000117713 (SEQ ID NO: 834).

By “basic leucine zipper transcription factor, ATF-like (BATF) polypeptide” is meant a protein having at least about 85% amino acid sequence identity to GenBank Accession No. AAC50314.1, which is provided below, or a fragment thereof having immunomodulatory activity.

>AAC50314.1 B-ATF [Homo sapiens]
(SEQ ID NO: 828)
MPHSSDSSDSSFSRSPPPGKQDSSDDVRRVQRREKNRIAAQKSRQ
RQTQKADTLHLESEDLEKQNAALRKEIKQLTEELKYFTSVLNSHE
PLCSVLAASTPSPPEVVYSAHAFHQPHVSSPRFQP.

By “basic leucine zipper transcription factor, ATF-like (BATF) polynucleotide” is meant a polynucleotide encoding a BATF polypeptide, as well as the introns, exons, 3′ untranslated regions, 5′ untranslated regions, and regulatory sequences associated with its expression, or fragments thereof. In embodiments, a BATF polynucleotide is the genomic sequence, cDNA, mRNA, or gene associated with and/or required for BATF expression. An exemplary BATF nucleic acid sequence is provided below (GenBank Accession No. U15460.1:210-587).

>U15460.1:210-587 Human bZip Protein B-ATF mRNA, Complete Cds
ATGCCTCACAGCTCCGACAGCAGTGACTCCAGCTTCAGCCGCTCTCCTCCCCCTGGCAAACA GGACTCATCTGATGATGTGAGAAGAGTTCAGAGGAGGGAGAAAAATCGTATTGCCGCCCAGA AGAGCCGACAGAGGCAGACACAGAAGGCCGACACCCTGCACCTGGAGAGCGAAGACCTGGAG AAACAGAACGCGGCTCTACGCAAGGAGATCAAGCAGCTCACAGAGGAACTGAAGTACTTCAC GTCGGTGCTGAACAGCCACGAGCCCCTGTGCTCGGTGCTGGCCGCCAGCACGCCCTCGCCCC CCGAGGTGGTGTACAGCGCCCACGCATTCCACCAACCTCATGTCAGCTCCCCGCGCTTCCAG CCCTGA (SEQ ID NO: 829). An exemplary BATF gene sequence is provided at ENSEMBL Accession No. ENSG00000156127 (SEQ ID NO: 835).

By “beta-2 microglobulin (β2M; B2M) polypeptide” is meant a protein having at least about 85% amino acid sequence identity to UniProt Accession No. P61769, which is provided below, or a fragment thereof having immunomodulatory activity.

>sp|P61769|B2MG_HUMAN Beta-2-microglobulin
OS = Homo sapiens
OX = 9606 GN = B2M
PE = 1 SV = 1
(SEQ ID NO: 553)
MSRSVALAVLALLSLSGLEAIQRTPKIQVYSRHPAENGKSNFLNC
YVSGFHPSDIEVDLLKNGERIEKVEHSDLSFSKDWSFYLLYYTEF
TPTEKDEYACRVNHVTLSQPKIVKWDRDM.

By “beta-2-microglobulin (β2M; B2M) polynucleotide” is meant a nucleic acid molecule encoding an β2M polypeptide, as well as the introns, exons, 3′ untranslated regions, 5′ untranslated regions, and regulatory sequences associated with its expression, or fragments thereof. The beta-2-microglobulin gene encodes a serum protein associated with the major histocompatibility complex. β2M is involved in non-self-recognition by host CD8+ T cells. An exemplary β2M polynucleotide sequence is provided at GenBank Accession No. DQ217933.1, which is provided below.

>DQ217933.1 Homo sapiens Beta-2-Microglobin (β2M) Gene, Complete Cds
CATGTCATAAATGGTAAGTCCAAGAAAAATACAGGTATTCCCCCCCAAAGAAAACTGTAAAA TCGACTTTTTTCTATCTGTACTGTTTTTTATTGGTTTTTAAATTGGTTTTCCAAGTGAGTAA ATCAGAATCTATCTGTAATGGATTTTAAATTTAGTGTTTCTCTGTGATGTAGTAAACAAGAA ACTAGAGGCAAAAATAGCCCTGTCCCTTGCTAAACTTCTAAGGCACTTTTCTAGTACAACTC AACACTAACATTTCAGGCCTTTAGTGCCTTATATGAGTTTTTAAAAGGGGGAAAAGGGAGGG AGCAAGAGTGTCTTAACTCATACATTTAGGCATAACAATTATTCTCATATTTTAGTTATTGA GAGGGCTGGTAGAAAAACTAGGTAAATAATATTAATAATTATAGCGCTTATTAAACACTACA GAACACTTACTATGTACCAGGCATTGTGGGAGGCTCTCTCTTGTGCATTATCTCATTTCATT AGGTCCATGGAGAGTATTGCATTTTCTTAGTTTAGGCATGGCCTCCACAATAAAGATTATCA AAAGCCTAAAAATATGTAAAAGAAACCTAGAAGTTATTTGTTGTGCTCCTTGGGGAAGCTAG GCAAATCCTTTCAACTGAAAACCATGGTGACTTCCAAGATCTCTGCCCCTCCCCATCGCCAT GGTCCACTTCCTCTTCTCACTGTTCCTCTTAGAAAAGATCTGTGGACTCCACCACCACGAAA TGGCGGCACCTTATTTATGGTCACTTTAGAGGGTAGGTTTTCTTAATGGGTCTGCCTGTCAT GTTTAACGTCCTTGGCTGGGTCCAAGGCAGATGCAGTCCAAACTCTCACTAAAATTGCCGAG CCCTTTGTCTTCCAGTGTCTAAAATATTAATGTCAATGGAATCAGGCCAGAGTTTGAATTCT AGTCTCTTAGCCTTTGTTTCCCCTGTCCATAAAATGAATGGGGGTAATTCTTTCCTCCTACA GTTTATTTATATATTCACTAATTCATTCATTCATCCATCCATTCGTTCATTCGGTTTACTGA GTACCTACTATGTGCCAGCCCCTGTTCTAGGGTGGAAACTAAGAGAATGATGTACCTAGAGG GCGCTGGAAGCTCTAAAGCCCTAGCAGTTACTGCTTTTACTATTAGTGGTCGTTTTTTTCTC CCCCCCGCCCCCCGACAATCAACAGAACAAAGAAAATTACCTAAACAGCAAGGACATAGGG AGGAACTTCTTGGCACAGAACTTTCCAAACACTTTTTCCTGAAGGGATACAAGAAGCAAGAA AGGTACTCTTTCACTAGGACCTTCTCTGAGCTGTCCTCAGGATGCTTTTGGGACTATTTTTC TTACCCAGAGAATGGAGAAACCCTGCAGGGAATTCCCAAGCTGTAGTTATAAACAGAAGTTC TCCTTCTGCTAGGTAGCATTCAAAGATCTTAATCTTCTGGGTTTCCGTTTTCTCGAATGAAA AATGCAGGTCCGAGCAGTTAACTGGCTGGGGCACCATTAGCAAGTCACTTAGCATCTCTGGG GCCAGTCTGCAAAGCGAGGGGGCAGCCTTAATGTGCCTCCAGCCTGAAGTCCTAGAATGAGC GCCCGGTGTCCCAAGCTGGGGCGCGCACCCCAGATCGGAGGGCGCCGATGTACAGACAGCAA ACTCACCCAGTCTAGTGCATGCCTTCTTAAACATCACGAGACTCTAAGAAAAGGAAACTGAA AACGGGAAAGTCCCTCTCTCTAACCTGGCACTGCGTCGCTGGCTTGGAGACAGGTGACGGTC CCTGCGGGCCTTGTCCTGATTGGCTGGGCACGCGTTTAATATAAGTGGAGGCGTCGCGCTGG CGGGCATTCCTGAAGCTGACAGCATTCGGGCCGAGATGTCTCGCTCCGTGGCCTTAGCTGTG CTCGCGCTACTCTCTCTTTCTGGCCTGGAGGCTATCCAGCGTGAGTCTCTCCTACCCTCCCG CTCTGGTCCTTCCTCTCCCGCTCTGCACCCTCTGTGGCCCTCGCTGTGCTCTCTCGCTCCGT GACTTCCCTTCTCCAAGTTCTCCTTGGTGGCCCGCCGTGGGGCTAGTCCAGGGCTGGATCTC GGGGAAGCGGCGGGGTGGCCTGGGAGTGGGGAAGGGGGTGCGCACCCGGGACGCGCGCTACT TGCCCCTTTCGGCGGGGAGCAGGGGAGACCTTTGGCCTACGGCGACGGGAGGGTCGGGACAA AGTTTAGGGCGTCGATAAGCGTCAGAGCGCCGAGGTTGGGGGAGGGTTTCTCTTCCGCTCTT TCGCGGGGCCTCTGGCTCCCCCAGCGCAGCTGGAGTGGGGGACGGGTAGGCTCGTCCCAAAG GCGCGGCGCTGAGGTTTGTGAACGCGTGGAGGGGCGCTTGGGGTCTGGGGGAGGCGTCGCCC GGGTAAGCCTGTCTGCTGCGGCTCTGCTTCCCTTAGACTGGAGAGCTGTGGACTTCGTCTAG GCGCCCGCTAAGTTCGCATGTCCTAGCACCTCTGGGTCTATGTGGGGCCACACCGTGGGGAG GAAACAGCACGCGACGTTTGTAGAATGCTTGGCTGTGATACAAAGCGGTTTCGAATAATTAA CTTATTTGTTCCCATCACATGTCACTTTTAAAAAATTATAAGAACTACCCGTTATTGACATC TTTCTGTGTGCCAAGGACTTTATGTGCTTTGCGTCATTTAATTTTGAAAACAGTTATCTTCC GCCATAGATAACTACTATGGTTATCTTCTGCCTCTCACAGATGAAGAAACTAAGGCACCGAG ATTTTAAGAAACTTAATTACACAGGGGATAAATGGCAGCAATCGAGATTGAAGTCAAGCCTA ACCAGGGCTTTTGCGGGAGCGCATGCCTTTTGGCTGTAATTCGTGCATTTTTTTTTAAGAAA AACGCCTGCCTTCTGCGTGAGATTCTCCAGAGCAAACTGGGCGGCATGGGCCCTGTGGTCTT TTCGTACAGAGGGCTTCCTCTTTGGCTCTTTGCCTGGTTGTTTCCAAGATGTACTGTGCCTC TTACTTTCGGTTTTGAAAACATGAGGGGGTTGGGCGTGGTAGCTTACGCCTGTAATCCCAGC ACTTAGGGAGGCCGAGGCGGGAGGATGGCTTGAGGTCCGTAGTTGAGACCAGCCTGGCCAAC ATGGTGAAGCCTGGTCTCTACAAAAAATAATAACAAAAATTAGCCGGGTGTGGTGGCTCGTG CCTGTGGTCCCAGCTGCTCCGGTGGCTGAGGCGGGAGGATCTCTTGAGCTTAGGCTTTTGAG CTATCATGGCGCCAGTGCACTCCAGCGTGGGCAACAGAGCGAGACCCTGTCTCTCAAAAAAG AAAAAAAAAAAAAAAGAAAGAGAAAAGAAAAGAAAGAAAGAAGTGAAGGTTTGTCAGTCAGG GGAGCTGTAAAACCATTAATAAAGATAATCCAAGATGGTTACCAAGACTGTTGAGGACGCCA GAGATCTTGAGCACTTTCTAAGTACCTGGCAATACACTAAGCGCGCTCACCTTTTCCTCTGG CAAAACATGATCGAAAGCAGAATGTTTTGATCATGAGAAAATTGCATTTAATTTGAATACAA TTTATTTACAACATAAAGGATAATGTATATATCACCACCATTACTGGTATTTGCTGGTTATG TTAGATGTCATTTTAAAAAATAACAATCTGATATTTAAAAAAAAATCTTATTTTGAAAATTT CCAAAGTAATACATGCCATGCATAGACCATTTCTGGAAGATACCACAAGAAACATGTAATGA TGATTGCCTCTGAAGGTCTATTTTCCTCCTCTGACCTGTGTGTGGGTTTTGTTTTTGTTTTA CTGTGGGCATAAATTAATTTTTCAGTTAAGTTTTGGAAGCTTAAATAACTCTCCAAAAGTCA TAAAGCCAGTAACTGGTTGAGCCCAAATTCAAACCCAGCCTGTCTGATACTTGTCCTCTTCT TAGAAAAGATTACAGTGATGCTCTCACAAAATCTTGCCGCCTTCCCTCAAACAGAGAGTTCC AGGCAGGATGAATCTGTGCTCTGATCCCTGAGGCATTTAATATGTTCTTATTATTAGAAGCT CAGATGCAAAGAGCTCTCTTAGCTTTTAATGTTATGAAAAAAATCAGGTCTTCATTAGATTC CCCAATCCACCTCTTGATGGGGCTAGTAGCCTTTCCTTAATGATAGGGTGTTTCTAGAGAGA TATATCTGGTCAAGGTGGCCTGGTACTCCTCCTTCTCCCCACAGCCTCCCAGACAAGGAGGA GTAGCTGCCTTTTAGTGATCATGTACCCTGAATATAAGTGTATTTAAAAGAATTTTATACAC ATATATTTAGTGTCAATCTGTATATTTAGTAGCACTAACACTTCTCTTCATTTTCAATGAAA AATATAGAGTTTATAATATTTTCTTCCCACTTCCCCATGGATGGTCTAGTCATGCCTCTCAT TTTGGAAAGTACTGTTTCTGAAACATTAGGCAATATATTCCCAACCTGGCTAGTTTACAGCA ATCACCTGTGGATGCTAATTTAAAACGCAAATCCCACTGTCACATGCATTACTCCATTTGATC ATAATGGAAAGTATGTTCTGTCCCATTTGCCATAGTCCTCACCTATCCCTGTTGTATTTTAT CGGGTCCAACTCAACCATTTAAGGTATTTGCCAGCTCTTGTATGCATTTAGGTTTTGTTTCT TTGTTTTTTAGCTCATGAAATTAGGTACAAAGTCAGAGAGGGGTCTGGCATATAAAACCTCA GCAGAAATAAAGAGGTTTTGTTGTTTGGTAAGAACATACCTTGGGTTGGTTGGGCACGGTGG CTCGTGCCTGTAATCCCAACACTTTGGGAGGCCAAGGCAGGCTGATCACTTGAAGTTGGGAG TTCAAGACCAGCCTGGCCAACATGGTGAAATCCCGTCTCTACTGAAAATACAAAAATTAACC AGGCATGGTGGTGTGTGCCTGTAGTCCCAGGAATCACTTGAACCCAGGAGGCGGAGGTTGCA GTGAGCTGAGATCTCACCACTGCACACTGCACTCCAGCCTGGGCAATGGAATGAGATTCCAT CCCAAAAAATAAAAAATAAAAAAATAAAGAACATACCTTGGGTTGATCCACTTAGGAACCT CAGATAATAACATCTGCCACGTATAGAGCAATTGCTATGTCCCAGGCACTCTACTAGACACT TCATACAGTTTAGAAAATCAGATGGGTGTAGATCAAGGCAGGAGCAGGAACCAAAAAGAAAG GCATAAACATAAGAAAAAAAATGGAAGGGGTGGAAACAGAGTACAATAACATGAGTAATTTG ATGGGGGCTATTATGAACTGAGAAATGAACTTTGAAAAGTATCTTGGGGCCAAATCATGTAG ACTCTTGAGTGATGTGTTAAGGAATGCTATGAGTGCTGAGAGGGCATCAGAAGTCCTTGAGA GCCTCCAGAGAAAGGCTCTTAAAAATGCAGCGCAATCTCCAGTGACAGAAGATACTGCTAGA AATCTGCTAGAAAAAAAACAAAAAAGGCATGTATAGAGGAATTATGAGGGAAAGATACCAAG TCACGGTTTATTCTTCAAAATGGAGGTGGCTTGTTGGGAAGGTGGAAGCTCATTTGGCCAGA GTGGAAATGGAATTGGGAGAAATCGATGACCAAATGTAAACACTTGGTGCCTGATATAGCTT GACACCAAGTTAGCCCCAAGTGAAATACCCTGGCAATATTAATGTGTCTTTTCCCGATATTC CTCAGGTACTCCAAAGATTCAGGTTTACTCACGTCATCCAGCAGAGAATGGAAAGTCAAATT TCCTGAATTGCTATGTGTCTGGGTTTCATCCATCCGACATTGAAGTTGACTTACTGAAGAAT GGAGAGAGAATTGAAAAAGTGGAGCATTCAGACTTGTCTTTCAGCAAGGACTGGTCTTTCTA TCTCTTGTACTACACTGAATTCACCCCCACTGAAAAAGATGAGTATGCCTGCCGTGTGAACC ATGTGACTTTGTCACAGCCCAAGATAGTTAAGTGGGGTAAGTCTTACATTCTTTTGTAAGCT GCTGAAAGTTGTGTATGAGTAGTCATATCATAAAGCTGCTTTGATATAAAAAAGGTCTATGG CCATACTACCCTGAATGAGTCCCATCCCATCTGATATAAACAATCTGCATATTGGGATTGTC AGGGAATGTTCTTAAAGATCAGATTAGTGGCACCTGCTGAGATACTGATGCACAGCATGGTT TCTGAACCAGTAGTTTCCCTGCAGTTGAGCAGGGAGCAGCAGCAGCACTTGCACAAATACAT ATACACTCTTAACACTTCTTACCTACTGGCTTCCTCTAGCTTTTGTGGCAGCTTCAGGTATA TTTAGCACTGAACGAACATCTCAAGAAGGTATAGGCCTTTGTTTGTAAGTCCTGCTGTCCTA GCATCCTATAATCCTGGACTTCTCCAGTACTTTCTGGCTGGATTGGTATCTGAGGCTAGTAG GAAGGGCTTGTTCCTGCTGGGTAGCTCTAAACAATGTATTCATGGGTAGGAACAGCAGCCTA TTCTGCCAGCCTTATTTCTAACCATTTTAGACATTTGTTAGTACATGGTATTTTAAAAGTAA AACTTAATGTCTTCCTTTTTTTTCTCCACTGTCTTTTTCATAGATCGAGACATGTAAGCAGC ATCATGGAGGTAAGTTTTTGACCTTGAGAAAATGTTTTTGTTTCACTGTCCTGAGGACTATT TATAGACAGCTCTAACATGATAACCCTCACTATGTGGAGAACATTGACAGAGTAACATTTTA GCAGGGAAAGAAGAATCCTACAGGGTCATGTTCCCTTCTCCTGTGGAGTGGCATGAAGAAGG TGTATGGCCCCAGGTATGGCCATATTACTGACCCTCTACAGAGAGGGCAAAGGAACTGCCAG TATGGTATTGCAGGATAAAGGCAGGTGGTTACCCACATTACCTGCAAGGCTTTGATCTTTCT TCTGCCATTTCCACATTGGACATCTCTGCTGAGGAGAGAAAATGAACCACTCTTTTCCTTTG TATAATGTTGTTTTATTCTTCAGACAGAAGAGAGGAGTTATACAGCTCTGCAGACATCCCAT TCCTGTATGGGGACTGTGTTTGCCTCTTAGAGGTTCCCAGGCCACTAGAGGAGATAAAGGGA AACAGATTGTTATAACTTGATATAATGATACTATAATAGATGTAACTACAAGGAGCTCCAGA AGCAAGAGAGAGGGAGGAACTTGGACTTCTCTGCATCTTTAGTTGGAGTCCAAAGGCTTTTC AATGAAATTCTACTGCCCAGGGTACATTGATGCTGAAACCCCATTCAAATCTCCTGTTATAT TCTAGAACAGGGAATTGATTTGGGAGAGCATCAGGAAGGTGGATGATCTGCCCAGTCACACT GTTAGTAAATTGTAGAGCCAGGACCTGAACTCTAATATAGTCATGTGTTACTTAATGACGGG GACATGTTCTGAGAAATGCTTACACAAACCTAGGTGTTGTAGCCTACTACACGCATAGGCTA CATGGTATAGCCTATTGCTCCTAGACTACAAACCTGTACAGCCTGTTACTGTACTGAATACT GTGGGCAGTTGTAACACAATGGTAAGTATTTGTGTATCTAAACATAGAAGTTGCAGTAAAAA TATGCTATTTTAATCTTATGAGACCACTGTCATATATACAGTCCATCATTGACCAAAACATC ATATCAGCATTTTTTCTTCTAAGATTTTGGGAGCACCAAAGGGATACACTAACAGGATATAC TCTTTATAATGGGTTTGGAGAACTGTCTGCAGCTACTTCTTTTAAAAAGGTGATCTACACAG TAGAAATTAGACAAGTTTGGTAATGAGATCTGCAATCCAAATAAAATAAATTCATTGCTAAC CTTTTTCTTTTCTTTTCAGGTTTGAAGATGCCGCATTTGGATTGGATGAATTCCAAATTCTG CTTGCTTGCTTTTTAATATTGATATGCTTATACACTTACACTTTATGCACAAAATGTAGGGT TATAATAATGTTAACATGGACATGATCTTCTTTATAATTCTACTTTGAGTGCTGTCTCCATG TTTGATGTATCTGAGCAGGTTGCTCCACAGGTAGCTCTAGGAGGGCTGGCAACTTAGAGGTG GGGAGCAGAGAATTCTCTTATCCAACATCAACATCTTGGTCAGATTTGAACTCTTCAATCTC TTGCACTCAAAGCTTGTTAAGATAGTTAAGCGTGCATAAGTTAACTTCCAATTTACATACTC TGCTTAGAATTTGGGGGAAAATTTAGAAATATAATTGACAGGATTATTGGAAATTTGTTATA ATGAATGAAACATTTTGTCATATAAGATTCATATTTACTTCTTATACATTTGATAAAGTAAG GCATGGTTGTGGTTAATCTGGTTTATTTTTGTTCCACAAGTTAAATAAATCATAAAACTTGA TGTGTTATCTCTTATATCTCACTCCCACTATTACCCCTTTATTTTCAAACAGGGAAACAGTC TTCAAGTTCCACTTGGTAAAAAATGTGAACCCCTTGTATATAGAGTTTGGCTCACAGTGTAA AGGGCCTCAGTGATTCACATTTTCCAGATTAGGAATCTGATGCTCAAAGAAGTTAAATGGCA TAGTTGGGGTGACACAGCTGTCTAGTGGGAGGCCAGCCTTCTATATTTTAGCCAGCGTTCTT TCCTGCGGGCCAGGTCATGAGGAGTATGCAGACTCTAAGAGGGAGCAAAAGTATCTGAAGGA TTTAATATTTTAGCAAGGAATAGATATACAATCATCCCTTGGTCTCCCTGGGGGATTGGTTT CAGGACCCCTTCTTGGACACCAAATCTATGGATATTTAAGTCCCTTCTATAAAATGGTATAG TATTTGCATATAACCTATCCACATCCTCCTGTATACTTTAAATCATTTCTAGATTACTTGTA ATACCTAATACAATGTAAATGCTATGCAAATAGTTGTTATTGTTTAAGGAATAATGACAAGA AAAAAAAGTCTGTACATGCTCAGTAAAGACACAACCATCCCTTTTTTTCCCCAGTGTTTTTG ATCCATGGTTTGCTGAATCCACAGATGTGGAGCCCCTGGATACGGAAGGCCCGCTGTACTTT GAATGACAAATAACAGATTTAAA (SEQ ID NO: 554). An exemplary B2M gene sequence is provided at (SEQ ID NO: 555).

By “CBL proto-oncogene B (CBLB) polypeptide” is meant a protein having at least about 85% amino acid sequence identity to GenBank Accession No. AAB09291.1, which is provided below, or a fragment thereof having immunomodulatory activity.

>AAB09291.1 cbl-b [Homo sapiens]
(SEQ ID NO: 556)
MANSMNGRNPGGRGGNPRKGRILGIIDAIQDAVGPPKQAAADRRT
VEKTWKLMDKVVRLCQNPKLQLKNSPPYILDILPDTYQHLRLILS
KYDDNQKLAQLSENEYFKIYIDSLMKKSKRAIRLFKEGKERMYEE
QSQDRRNLTKLSLIFSHMLAEIKAIFPNGQFQGDNFRITKADAAE
FWRKFFGDKTIVPWKVFRQCLHEVHQISSSLEAMALKSTIDLTCN
DYISVFEFDIFTRLFQPWGSILRNWNFLAVTHPGYMAFLTYDEVK
ARLQKYSTKPGSYIFRLSCTRLGQWAIGYVTGDGNILQTIPHNKP
LFQALIDGSREGFYLYPDGRSYNPDLTGLCEPTPHDHIKVTQEQY
ELYCEMGSTFQLCKICAENDKDVKIEPCGHLMCTSCLTAWQESDG
QGCPFCRCEIKGTEPIIVDPFDPRDEGSRCCSIIDPFGMPMLDLD
DDDDREESLMMNRLANVRKCTDRQNSPVTSPGSSPLAQRRKPQPD
PLQIPHLSLPPVPPRLDLIQKGIVRSPCGSPTGSPKSSPCMVRKQ
DKPLPAPPPPLRDPPPPPPERPPPIPPDNRLSRHIHHVESVPSRD
PPMPLEAWCPRDVFGTNQLVGCRLLGEGSPKPGITASSNVNGRHS
RVGSDPVLMRKHRRHDLPLEGAKVFSNGHLGSEEYDVPPRLSPPP
PVTTLLPSIKCTGPLANSLSEKTRDPVEEDDDEYKIPSSHPVSLN
SQPSHCHNVKPPVRSCDNGHCMLNGTHGPSSEKKSNIPDLSIYLK
GDVFDSASDPVPLPPARPPTRDNPKHGSSLNRTPSDYDLLIPPLG
EDAFDALPPSLPPPPPPARHSLIEHSKPPGSSSRPSSGQDLFLLP
SDPFVDLASGQVPLPPARRLPGENVKTNRTSQDYDQLPSCSDGSQ
APARPPKPRPRRTAPEIHHRKPHGPEAALENVDAKIAKLMGEGYA
FEEVKRALEIAQNNVEVARSILREFAFPPPVSPRLNL.

By “CBL proto-oncogene B (CBLB) polynucleotide” is meant a polynucleotide encoding a CBLB polypeptide, as well as the introns, exons, 3′ untranslated regions, 5′ untranslated regions, and regulatory sequences associated with its expression, or fragments thereof. In embodiments, a CBLB polynucleotide is the genomic sequence, cDNA, mRNA, or gene associated with and/or required for CBLB expression. An exemplary CBLB nucleic acid sequence is provided below (GenBank Accession No. U26710.1:323-3271).

>U26710.1:323-3271 Human Cbl-b mRNA, Complete Cds
ATGGCAAACTCAATGAATGGCAGAAACCCTGGTGGTCGAGGAGGAAATCCCCGAAAAGGTCG AATTTTGGGTATTATTGATGCTATTCAGGATGCAGTTGGACCCCCTAAGCAAGCTGCCGCAG ATCGCAGGACCGTGGAGAAGACTTGGAAGCTCATGGACAAAGTGGTAAGACTGTGCCAAAAT CCCAAACTTCAGTTGAAAAATAGCCCACCATATATACTTGATATTTTGCCTGATACATATCA GCATTTACGACTTATATTGAGTAAATATGATGACAACCAGAAACTTGCCCAACTCAGTGAGA ATGAGTACTTTAAAATCTACATTGATAGCCTTATGAAAAAGTCAAAACGGGCAATAAGACTC TTTAAAGAAGGCAAGGAGAGAATGTATGAAGAACAGTCACAGGACAGACGAAATCTCACAAA ACTGTCCCTTATCTTCAGTCACATGCTGGCAGAAATCAAAGCAATCTTTCCCAATGGTCAAT TCCAGGGAGATAACTTTCGTATCACAAAAGCAGATGCTGCTGAATTCTGGAGAAAGTTTTTT GGAGACAAAACTATCGTACCATGGAAAGTATTCAGACAGTGCCTTCATGAGGTCCACCAGAT TAGCTCTAGCCTGGAAGCAATGGCTCTAAAATCAACAATTGATTTAACTTGCAATGATTACA TTTCAGTTTTTGAATTTGATATTTTTACCAGGCTGTTTCAGCCTTGGGGCTCTATTTTGCGG AATTGGAATTTCTTAGCTGTGACACATCCAGGTTACATGGCATTTCTCACATATGATGAAGT TAAAGCACGACTACAGAAATATAGCACCAAACCCGGAAGCTATATTTTCCGGTTAAGTTGCA CTCGATTGGGACAGTGGGCCATTGGCTATGTGACTGGGGATGGGAATATCTTACAGACCATA CCTCATAACAAGCCCTTATTTCAAGCCCTGATTGATGGCAGCAGGGAAGGATTTTATCTTTA TCCTGATGGGAGGAGTTATAATCCTGATTTAACTGGATTATGTGAACCTACACCTCATGACC ATATAAAAGTTACACAGGAACAATATGAATTATATTGTGAAATGGGCTCCACTTTTCAGCTC TGTAAGATTTGTGCAGAGAATGACAAAGATGTCAAGATTGAGCCTTGTGGGCATTTGATGTG CACCTCTTGCCTTACGGCATGGCAGGAGTCGGATGGTCAGGGCTGCCCTTTCTGTCGTTGTG AAATAAAAGGAACTGAGCCCATAATCGTGGACCCCTTTGATCCAAGAGATGAAGGCTCCAGG TGTTGCAGCATCATTGACCCCTTTGGCATGCCGATGCTAGACTTGGACGACGATGATGATCG TGAGGAGTCCTTGATGATGAATCGGTTGGCAAACGTCCGAAAGTGCACTGACAGGCAGAACT CACCAGTCACATCACCAGGATCCTCTCCCCTTGCCCAGAGAAGAAAGCCACAGCCTGACCCA CTCCAGATCCCACATCTAAGCCTGCCACCCGTGCCTCCTCGCCTGGATCTAATTCAGAAAGG CATAGTTAGATCTCCCTGTGGCAGCCCAACAGGTTCACCAAAGTCTTCTCCTTGCATGGTGA GAAAACAAGATAAACCACTCCCAGCACCACCTCCTCCCTTAAGAGATCCTCCTCCACCGCCA CCTGAAAGACCTCCACCAATCCCACCAGACAATAGACTGAGTAGACACATCCATCATGTGGA AAGCGTGCCTTCCAGAGACCCGCCAATGCCTCTTGAAGCATGGTGCCCTCGGGATGTGTTTG GGACTAATCAGCTTGTGGGATGTCGACTCCTAGGGGAGGGCTCTCCAAAACCTGGAATCACA GCGAGTTCAAATGTCAATGGAAGGCACAGTAGAGTGGGCTCTGACCCAGTGCTTATGCGGAA ACACAGACGCCATGATTTGCCTTTAGAAGGAGCTAAGGTCTTTTCCAATGGTCACCTTGGAA GTGAAGAATATGATGTTCCTCCCCGGCTTTCTCCTCCTCCTCCAGTTACCACCCTCCTCCCT AGCATAAAGTGTACTGGTCCGTTAGCAAATTCTCTTTCAGAGAAAACAAGAGACCCAGTAGA GGAAGATGATGATGAATACAAGATTCCTTCATCCCACCCTGTTTCCCTGAATTCACAACCAT CTCATTGTCATAATGTAAAACCTCCTGTTCGGTCCTGTGATAATGGTCACTGTATGCTGAAT GGAACACATGGTCCATCTTCAGAGAAGAAATCAAACATCCCTGACTTAAGCATATATTTAAA GGGAGATGTTTTTGATTCAGCCTCTGATCCCGTGCCATTACCACCTGCCAGGCCTCCAACTC GGGACAATCCAAAGCATGGTTCTTCACTCAACAGGACGCCCTCTGATTATGATCTTCTCATC CCTCCATTAGGTGAAGATGCTTTTGATGCCCTCCCTCCATCTCTCCCACCTCCCCCACCTCC TGCAAGGCATAGTCTCATTGAACATTCAAAACCTCCTGGCTCCAGTAGCCGGCCATCCTCAG GACAGGATCTTTTTCTTCTTCCTTCAGATCCCTTTGTTGATCTAGCAAGTGGCCAAGTTCCT TTGCCTCCTGCTAGAAGGTTACCAGGTGAAAATGTCAAAACTAACAGAACATCACAGGACTA TGATCAGCTTCCTTCATGTTCAGATGGTTCACAGGCACCAGCCAGACCCCCTAAACCACGAC CGCGCAGGACTGCACCAGAAATTCACCACAGAAAACCCCATGGGCCTGAGGCGGCATTGGAA AATGTCGATGCAAAAATTGCAAAACTCATGGGAGAGGGTTATGCCTTTGAAGAGGTGAAGAG AGCCTTAGAGATAGCCCAGAATAATGTCGAAGTTGCCCGGAGCATCCTCCGAGAATTTGCCT TCCCTCCTCCAGTATCCCCACGTCTAAATCTATAG (SEQ ID NO: 557). An exemplary CBLB gene sequence is provided at ENSEMBL Accession No. ENSG00000114423 (SEQ ID NO: 558).

By “cluster of differentiation 3 epsilon (CD3e or CD3 epsilon) polypeptide” is meant a protein having at least about 85% amino acid sequence identity to NCBI Accession No. NP_000724.1 or a fragment thereof having immunomodulatory activity. An exemplary amino acid sequence is provided below.

>NP_000724.1 T-cell surface glycoprotein CD3
epsilon chain precursor [Homo sapiens]
(SEQ ID NO: 559)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI.

By “cluster of differentiation 3 epsilon (CD3e or CD3 epsilon) polynucleotide” is meant a polynucleotide encoding a CD3e polypeptide, as well as the introns, exons, 3′ untranslated regions, 5′ untranslated regions, and regulatory sequences associated with its expression, or fragments thereof. In embodiments, a CD3e polynucleotide is the genomic sequence, cDNA, mRNA, or gene associated with and/or required for CD3e expression. An exemplary CD3e nucleic acid sequence is provided below (NCBI Ref. Seq. Accession No. NM_000733.4).

>NM_000733.4 Homo sapiens CD3e Molecule (CD3E), mRNA
AGAAACCCTCCTCCCCTCCCAGCCTCAGGTGCCTGCTTCAGAAAATGAAGTAGTAAGTCTGC TGGCCTCCGCCATCTTAGTAAAGTAACAGTCCCATGAAACAAAGATGCAGTCGGGCACTCAC TGGAGAGTTCTGGGCCTCTGCCTCTTATCAGTTGGCGTTTGGGGGCAAGATGGTAATGAAGA AATGGGTGGTATTACACAGACACCATATAAAGTCTCCATCTCTGGAACCACAGTAATATTGA CATGCCCTCAGTATCCTGGATCTGAAATACTATGGCAACACAATGATAAAAACATAGGCGGT GATGAGGATGATAAAAACATAGGCAGTGATGAGGATCACCTGTCACTGAAGGAATTTTCAGA ATTGGAGCAAAGTGGTTATTATGTCTGCTACCCCAGAGGAAGCAAACCAGAAGATGCGAACT TTTATCTCTACCTGAGGGCAAGAGTGTGTGAGAACTGCATGGAGATGGATGTGATGTCGGTG GCCACAATTGTCATAGTGGACATCTGCATCACTGGGGGCTTGCTGCTGCTGGTTTACTACTG GAGCAAGAATAGAAAGGCCAAGGCCAAGCCTGTGACACGAGGAGCGGGTGCTGGCGGCAGGC AAAGGGGACAAAACAAGGAGAGGCCACCACCTGTTCCCAACCCAGACTATGAGCCCATCCGG AAAGGCCAGCGGGACCTGTATTCTGGCCTGAATCAGAGACGCATCTGACCCTCTGGAGAACA CTGCCTCCCGCTGGCCCAGGTCTCCTCTCCAGTCCCCCTGCGACTCCCTGTTTCCTGGGCTA GTCTTGGACCCCACGAGAGAGAATCGTTCCTCAGCCTCATGGTGAACTCGCGCCCTCCAGCC TGATCCCCCGCTCCCTCCTCCCTGCCTTCTCTGCTGGTACCCAGTCCTAAAATATTGCTGCT TCCTCTTCCTTTGAAGCATCATCAGTAGTCACACCCTCACAGCTGGCCTGCCCTCTTGCCAG GATATTTATTTGTGCTATTCACTCCCTTCCCTTTGGATGTAACTTCTCCGTTCAGTTCCCTC CTTTTCTTGCATGTAAGTTGTCCCCCATCCCAAAGTATTCCATCTACTTTTCTATCGCCGTC CCCTTTTGCAGCCCTCTCTGGGGATGGACTGGGTAAAATGTTGACAGAGGCCCTGCCCCGTTC ACAGATCCTGGCCCTGAGCCAGCCCTGTGCTCCTCCCTCCCCCAACACTCCCTACCAACCCC CTAATCCCCTACTCCCTCCACCCCCCCTCCACTGTAGGCCACTGGATGGTCATTTGCATCTC CGTAAATGTGCTCTGCTCCTCAGCTGAGAGAGAAAAAAATAAACTGTATTTGGCTGCAA (SEQ ID NO: 560). An exemplary CD3e sequence is provided at ENSEMBL Accession No. ENSG00000198851 (SEQ ID NO: 561).

By “cluster of differentiation 3 gamma (CD3g or CD3 gamma) polypeptide” is meant a protein having at least about 85% amino acid sequence identity to NCBI Accession No. NP_000064.1 or a fragment thereof and having immunomodulatory activity. An exemplary amino acid sequence is provided below.

>NP_000064.1 T-cell surface glycoprotein CD3
gamma chain precursor [Homo sapiens]
(SEQ ID NO: 562)
MEQGKGLAVLILAIILLQGTLAQSIKGNHLVKVYDYQEDGSVLLT
CDAEAKNITWFKDGKMIGFLTEDKKKWNLGSNAKDPRGMYQCKGS
QNKSKPLQVYYRMCQNCIELNAATISGFLFAEIVSIFVLAVGVYF
IAGQDGVRQSRASDKQTLLPNDQLYQPLKDREDDQYSHLQGNQLR
RN.

By “cluster of differentiation 3 gamma (CD3g or CD3 gamma) polynucleotide” is meant a polypeptide encoding a CD3g polypeptide, as well as the introns, exons, 3′ untranslated regions, 5′ untranslated regions, and regulatory sequences associated with its expression, or fragments thereof. In embodiments, a CD3g polynucleotide is the genomic sequence, cDNA, mRNA, or gene associated with and/or required for CD3g expression. An exemplary CD3g nucleic acid sequence is provided below (NCBI Ref. Seq. Accession No. NM_000073.3).

>NM_000073.3 Homo sapiens CD3g Molecule (CD3G), mRNA
AGTCTAGCTGCTGCACAGGCTGGCTGGCTGGCTGGCTGCTAAGGGCTGCTCCACGCTTTTGC CGGAGGACAGAGACTGACATGGAACAGGGGAAGGGCCTGGCTGTCCTCATCCTGGCTATCAT TCTTCTTCAAGGTACTTTGGCCCAGTCAATCAAAGGAAACCACTTGGTTAAGGTGTATGACT ATCAAGAAGATGGTTCGGTACTTCTGACTTGTGATGCAGAAGCCAAAAATATCACATGGTTT AAAGATGGGAAGATGATCGGCTTCCTAACTGAAGATAAAAAAAAATGGAATCTGGGAAGTAA TGCCAAGGACCCTCGAGGGATGTATCAGTGTAAAGGATCACAGAACAAGTCAAAACCACTCC AAGTGTATTACAGAATGTGTCAGAACTGCATTGAACTAAATGCAGCCACCATATCTGGCTTT CTCTTTGCTGAAATCGTCAGCATTTTCGTCCTTGCTGTTGGGGTCTACTTCATTGCTGGACA GGATGGAGTTCGCCAGTCGAGAGCTTCAGACAAGCAGACTCTGTTGCCCAATGACCAGCTCT ACCAGCCCCTCAAGGATCGAGAAGATGACCAGTACAGCCACCTTCAAGGAAACCAGTTGAGG AGGAATTGAACTCAGGACTCAGAGTAGTCCAGGTGTTCTCCTCCTATTCAGTTCCCAGAATC AAAGCAATGCATTTTGGAAAGCTCCTAGCAGAGAGACTTTCAGCCCTAAATCTAGACTCAAG GTTCCCAGAGATGACAAATGGAGAAGAAAGGCCATCAGAGCAAATTTGGGGGTTTCTCAAAT AAAATAAAAATAAAAACAAATACTGTGTTTCAGAAGCGCCACCTATTGGGGAAAATTGTAAA AGAAAAATGAAAAGATCAAATAACCCCCTGGATTTGAATATAATTTTTTGTGTTGTAATTTT TATTTCGTTTTTGTATAGGTTATAATTCACATGGCTCAAATATTCAGTGAAAGCTCTCCCTC CACCGCCATCCCCTGCTACCCAGTGACCCTGTTGCCCTCTTCAGAGACAAATTAGTTTCTCT TTTTTTTTTTTTTTTTTTTTTTTTTGAGACAGTCTGGCTCTGTCACCCAGGCTGAAATGCAG TGGCACCATCTCGGCTCACTGCAACCTCTGCCTCCTGGGTTCAAGCGATTCTCCTGCCTCAG CCTCCCGGGCAGCTGGGATTACAGGCACACACTACCACACCTGGCTAATTTTTGTATTTTTA GTAGAGACAGGGTTTTGCTCTGTTGGCCAAGCTGGTCTCGAACTCCTGACCTCAAGTGATCC GCCCGCCTCAGCCTCCCAAAGTGCTGGGATTACAGGTGTGAGCCACCATGCCTGGTCTTAAA ACCAGTTTCTTATATATCTCTCTGGAGGTATTCTAGGCATATATGAGCACATTCTCAAGTAC ATATTATCCTCCCTTCCCCTATCTTTTAGACAAATGATATCAAACTATACATCTTGTGAGAT TATTGCATACCATTATATGAAGATACCATTATATCCTTTTTAATGCAACCATATTGTACAAA TAGACTATGATTTATTTAACCTGTTATCTATCAGTGGATATTTAAGTTGGTAGTTGGTTCCA ATCTTTTGCTCTTACAACAATTCTGCAATGACTAACATTGTATAAATATCATTTTTAAAAAT AATTGCATTGAAGCATAATGTACATGCCATAAAATCCACCCATCTTAAGTGATTTCACCTGT TCTCAGAAATTTTTAGTAAATTTAACTAATTGTACAGCCATTACCATAATCCAGCTTTAGGA CATTTTCTTTTTTTTCTTTTCTTTTCTTTTTTTTCTTTTTTTTTTTTTTTTGAAGTGGAATC TTGCTCTGTGGCCCAGGCTGGAGTGCAGTGGCGCGATCTCAGCTCACTGCAACCTCCACCTC CTGGGTTCAAGCGATTCTCTTGCCTTGGCCTCCCGAGTAGCTGAGACTACAGGCACATGCCA CCACGCCCAGCTCATTTTTTGTGTATTTAGTATTTGTGTATCTAGTATTTGTGTACTTAGTA GAGACAGGGTTTCACCATGTTGGCCAGGCTGGTCTCCAATTCCTGACCTCAGGCGATCCACC CGCCTTGACCTCCCAAAGTGCTGGGATTACAGGTGTGAGCCACCGCGCCAGGCCCGTAACTG TATTTTAATATAGCCATTCTATGGATTTAATATGGTATTTTATTATGGCCTTAATTTGCATT TCCCTAGATACTAACCATGCTGAGTGTCCTGTCTTGTGTTTATTAACCATTCATATATTTTT AGTGAAATGTGTATCAAATCTTTTGCCCATTTTTAAGTTGACTTATTTGTTTGTCTTCTTAC TATTGGGTTGCATATGTTTTTGATATAAGTCCTTTATCAGATATATGATTTGGAAATATTTT CTACCAATCTGTGGTTTGTTTTTCTTAATGGTGTCTTTTGAAGTGCAAAAGGTTTGAATTTT GAAGTACATTTTATTGATTTTTTCTTCTATATATTGTGCTTTTGGTATCATGTCTAATAAAT CTTTACCAAACCCACAGTTACAAAGATTTTCTCCTGTCTTCTTTTTATACTTTTTACAGCTT TATGGTTTTAGCTCTAACAATAAATGTGATTTTGAACATACATAAGACTATTTGTAACAAAC ACAAATAAATTGAATTGTTGGGCA (SEQ ID NO: 563). An exemplary CD3g sequence is provided at ENSEMBL Accession No. ENSG00000160654 (SEQ ID NO: 564).

By “cluster of differentiation 5 (CD5) polypeptide” is meant a protein having at least about 85% amino acid sequence identity to NCBI Accession No. NP_001333385.1, which is provided below, or a fragment thereof having immunomodulatory activity.

>NP_001333385.1 T-cell surface glycoprotein
CD5 isoform 2 [Homo sapiens]
(SEQ ID NO: 565)
MVCSQSWGRSSKQWEDPSQASKVCQRLNCGVPLSLGPFLVTYTPQ
SSIICYGQLGSFSNCSHSRNDMCHSLGLTCLEPQKTTPPTTRPPP
TTTPEPTAPPRLQLVAQSGGQHCAGVVEFYSGSLGGTISYEAQDK
TQDLENFLCNNLQCGSFLKHLPETEAGRAQDPGEPREHQPLPIQW
KIQNSSCTSLEHCFRKIKPQKSGRVLALLCSGFQPKVQSRLVGGS
SICEGTVEVRQGAQWAALCDSSSARSSLRWEEVCREQQCGSVNSY
RVLDAGDPTSRGLFCPHQKLSQCHELWERNSYCKKVFVTCQDPNP
AGLAAGTVASIILALVLLVVLLVVCGPLAYKKLVKKFRQKKQRQW
IGPTGMNQNMSFHRNHTATVRSHAENPTASHVDNEYSQPPRNSHL
SAYPALEGALHRSSMQPDNSSDSDYDLHGAQRL

By “cluster of differentiation 5 (CD5) polynucleotide” is meant a polynucleotide encoding a CD5 polypeptide, as well as the introns, exons, 3′ untranslated regions, 5′ untranslated regions, and regulatory sequences associated with its expression, or fragments thereof. In embodiments, a CD5 polynucleotide is the genomic sequence, cDNA, mRNA, or gene associated with and/or required for CD5 expression. An exemplary CD5 nucleic acid sequence is provided below (NCBI Ref. Seq. Accession No. NM_001346456.1).

>NM_001346456.1 Homo sapiens CD5 Molecule (CD5), Transcript Variant 2, mRNA
GAGTCTTGCTGATGCTCCCGGCTGAATAAACCCCTTCCTTCTTTAACTTGGTGTCTGAGGGG TTTTGTCTGTGGCTTGTCCTGCTACATTTCTTGGTTCCCTGACCAGGAAGCAAAGTGATTAA CGGACAGTTGAGGCAGCCCCTTAGGCAGCTTAGGCCTGCCTTGTGGAGCATCCCCGCGGGGA ACTCTGGCCAGCTTGAGCGACACGGATCCTCAGAGCGCTCCCAGGTAGGCAATTGCCCCAGT GGAATGCCTCGTCAGAGCAGTGCATGGCAGGCCCCTGTGGAGGATCAACGCAGTGGCTGAAC ACAGGGAAGGAACTGGCACTTGGAGTCCGGACAACTGAAACTTGTCGCTTCCTGCCTCGGAC GGCTCAGCTGGTATGACCCAGATTTCCAGGCAAGGCTCACCCGTTCCAACTCGAAGTGCCAG GGCCAGCTGGAGGTCTACCTCAAGGACGGATGGCACATGGTTTGCAGCCAGAGCTGGGGCCG GAGCTCCAAGCAGTGGGAGGACCCCAGTCAAGCGTCAAAAGTCTGCCAGCGGCTGAACTGTG GGGTGCCCTTAAGCCTTGGCCCCTTCCTTGTCACCTACACACCTCAGAGCTCAATCATCTGC TACGGACAACTGGGCTCCTTCTCCAACTGCAGCCACAGCAGAAATGACATGTGTCACTCTCT GGGCCTGACCTGCTTAGAACCCCAGAAGACAACACCTCCAACGACAAGGCCCCCGCCCACCA CAACTCCAGAGCCCACAGCTCCTCCCAGGCTGCAGCTGGTGGCACAGTCTGGCGGCCAGCAC TGTGCCGGCGTGGTGGAGTTCTACAGCGGCAGCCTGGGGGGTACCATCAGCTATGAGGCCCA GGACAAGACCCAGGACCTGGAGAACTTCCTCTGCAACAACCTCCAGTGTGGCTCCTTCTTGA AGCATCTGCCAGAGACTGAGGCAGGCAGAGCCCAAGACCCAGGGGAGCCACGGGAACACCAG CCCTTGCCAATCCAATGGAAGATCCAGAACTCAAGCTGTACCTCCCTGGAGCATTGCTTCAG GAAAATCAAGCCCCAGAAAAGTGGCCGAGTTCTTGCCCTCCTTTGCTCAGGTTTCCAGCCCA AGGTGCAGAGCCGTCTGGTGGGGGGCAGCAGCATCTGTGAAGGCACCGTGGAGGTGCGCCAG GGGGCTCAGTGGGCAGCCCTGTGTGACAGCTCTTCAGCCAGGAGCTCGCTGCGGTGGGAGGA GGTGTGCCGGGAGCAGCAGTGTGGCAGCGTCAACTCCTATCGAGTGCTGGACGCTGGTGACC CAACATCCCGGGGGCTCTTCTGTCCCCATCAGAAGCTGTCCCAGTGCCACGAACTTTGGGAG AGAAATTCCTACTGCAAGAAGGTGTTTGTCACATGCCAGGATCCAAACCCCGCAGGCCTGGC CGCAGGCACGGTGGCAAGCATCATCCTGGCCCTGGTGCTCCTGGTGGTGCTGCTGGTCGTGT GCGGCCCCCTTGCCTACAAGAAGCTAGTGAAGAAATTCCGCCAGAAGAAGCAGCGCCAGTGG ATTGGCCCAACGGGAATGAACCAAAACATGTCTTTCCATCGCAACCACACGGCAACCGTCCG ATCCCATGCTGAGAACCCCACAGCCTCCCACGTGGATAACGAATACAGCCAACCTCCCAGGA ACTCCCACCTGTCAGCTTATCCAGCTCTGGAAGGGGCTCTGCATCGCTCCTCCATGCAGCCT GACAACTCCTCCGACAGTGACTATGATCTGCATGGGGCTCAGAGGCTGTAAAGAACTGGGAT CCATGAGCAAAAAGCCGAGAGCCAGACCTGTTTGTCCTGAGAAAACTGTCCGCTCTTCACTT GAAATCATGTCCCTATTTCTACCCCGGCCAGAACATGGACAGAGGCCAGAAGCCTTCCGGAC AGGCGCTGCTGCCCCGAGTGGCAGGCCAGCTCACACTCTGCTGCACAACAGCTCGGCCGCCC CTCCACTTGTGGAAGCTGTGGTGGGCAGAGCCCCAAAACAAGCAGCCTTCCAACTAGAGACT CGGGGGTGTCTGAAGGGGGCCCCCTTTCCCTGCCCGCTGGGGAGCGGCGTCTCAGTGAAATC GGCTTTCTCCTCAGACTCTGTCCCTGGTAAGGAGTGACAAGGAAGCTCACAGCTGGGCGAGT GCATTTTGAATAGTTTTTTGTAAGTAGTGCTTTTCCTCCTTCCTGACAAATCGAGCGCTTTG GCCTCTTCTGTGCAGCATCCACCCCTGCGGATCCCTCTGGGGAGGACAGGAAGGGGACTCCC GGAGACCTCTGCAGCCGTGGTGGTCAGAGGCTGCTCACCTGAGCACAAAGACAGCTCTGCAC ATTCACCGCAGCTGCCAGCCAGGGGTCTGGGTGGGCACCACCCTGACCCACAGCGTCACCCC ACTCCCTCTGTCTTATGACTCCCCTCCCCAACCCCCTCATCTAAAGACACCTTCCTTTCCAC TGGCTGTCAAGCCCACAGGGCACCAGTGCCACCCAGGGCCCGGCACAAAGGGGCGCCTAGTA AACCTTAACCAACTTGGTTTTTTGCTTCACCCAGCAATTAAAAGTCCCAAGCTGAGGTAGTT TCAGTCCATCACAGTTCATCTTCTAACCCAAGAGTCAGAGATGGGGCTGGTCATGTTCCTTT GGTTTGAATAACTCCCTTGACGAAAACAGACTCCTCTAGTACTTGGAGATCTTGGACGTACA CCTAATCCCATGGGGCCTCGGCTTCCTTAACTGCAAGTGAGAAGAGGAGGTCTACCCAGGAG CCTCGGGTCTGATCAAGGGAGAGGCCAGGCGCAGCTCACTGCGGCGGCTCCCTAAGAAGGTG AAGCAACATGGGAACACATCCTAAGACAGGTCCTTTCTCCACGCCATTTGATGCTGTATCTC CTGGGAGCACAGGCATCAATGGTCCAAGCCGCATAATAAGTCTGGAAGAGCAAAAGGGAGTT ACTAGGATATGGGGTGGGCTGCTCCCAGAATCTGCTCAGCTTTCTGCCCCCACCAACACCCT CCAACCAGGCCTTGCCTTCTGAGAGCCCCCGTGGCCAAGCCCAGGTCACAGATCTTCCCCCG ACCATGCTGGGAATCCAGAAACAGGGACCCCATTTGTCTTCCCATATCTGGTGGAGGTGAGG GGGCTCCTCAAAAGGGAACTGAGAGGCTGCTCTTAGGGAGGGCAAAGGTTCGGGGGCAGCCA GTGTCTCCCATCAGTGCCTTTTTTAATAAAAGCTCTTTCATCTATAGTTTGGCCACCATACA GTGGCCTCAAAGCAACCATGGCCTACTTAAAAACCAAACCAAAAATAAAGAGTTTAGTTGAG GAGAAAAAAAAAAAAAAAAAAAAAAAAA (SEQ ID NO: 566). An exemplary CD5 gene sequence is provided at ENSEMBL Accession No. ENSG00000110448 (SEQ ID NO: 567).

By “B-lymphocyte antigen CD19 (CD19) polypeptide” is meant a protein having at least about 85% amino acid sequence identity to GenBank Accession No.: AAB60697.1, which is provided below, or a fragment thereof having immunomodulatory activity.

>AAB60697.1 CD19 [Homo sapiens]
(SEQ ID NO: 568)
MPPPRLLFFLLFLTPMEVRPEEPLVVKVEGEGDNAVLQCLKGTSD
GPTQQLTWSRESPLKPFLKLSLGLPGLGIHMRPLASWLFIFNVSQ
QMGGFYLCQPGPPSEKAWQPGWTVNVEGSGELFRWNVSDLGGLGC
GLKNRSSEGPSSPSGKLMSPKLYVWAKDRPEIWEGEPPCVPPRDS
LNQSLSQDLTMAPGSTLWLSCGVPPDSVSRGPLSWTHVHPKGPKS
LLSLELKDDRPARDMWVMETGLLLPRATAQDAGKYYCHRGNLTMS
FHLEITARPVLWHWLLRTGGWKVSAVTLAYLIFCLCSLVGILHLQ
RALVLRRKRKRMTDPTRRFFKVTPPPGSGPQNQYGNVLSLPTPTS
GLGRAQRWAAGLGGTAPSYGNPSSDVQADGALGSRSPPGVGPEEE
EGEGYEEPDSEEDSEFYENDSNLGQDQLSQDGSGYENPEDEPLGP
EDEDSFSNAESYENEDEELTQPVARTMDFLSPHGSAWDPSREATS
LGSQSYEDMRGILYAAPQLRSIRGQPGPNHEEDADSYENMDNPDG
PDPAWGGGGRMGTWSTR

By “B-lymphocyte antigen CD19 (CD19) polynucleotide” is meant a polynucleotide encoding a CD19 polypeptide, as well as the introns, exons, 3′ untranslated regions, 5′ untranslated regions, and regulatory sequences associated with its expression, or fragments thereof. In embodiments, a CD19 polynucleotide is the genomic sequence, cDNA, mRNA, or gene associated with and/or required for CD19 expression. An exemplary CD19 nucleic acid sequence is provided below (GenBank Accession No. AH005421.2:331-421,665-931,1230-1433,1554-1829,2057-2167,2769-2817,3128-3216,3591-3704,3896-4000,4174-4242,4343-4399,4488-4544,4813-4905,5231-5322).

>AH005421.2:331-421,665-931,1230-1433,1554-1829,2057-2167,2769-2817,3128-3216,3591-3704,3896-4000,4174-4242,4343-4399,4488-4544,4813-4905,5231-5322 Homo sapiens CD19 (CD19) Gene, Complete Cds
ATGCCACCTCCTCGCCTCCTCTTCTTCCTCCTCTTCCTCACCCCCATGGAAGTCAGGCCCGA GGAACCTCTAGTGGTGAAGGTGGAAGGTGAGGGAGATAACGCTGTGCTGCAGTGCCTCAAGG GGACCTCAGATGGCCCCACTCAGCAGCTGACCTGGTCTCGGGAGTCCCCGCTTAAACCCTTC TTAAAACTCAGCCTGGGGCTGCCAGGCCTGGGAATCCACATGAGGCCCCTGGCATCCTGGCT TTTCATCTTCAACGTCTCTCAACAGATGGGGGGCTTCTACCTGTGCCAGCCGGGGCCCCCCT CTGAGAAGGCCTGGCAGCCTGGCTGGACAGTCAATGTGGAGGGCAGCGGGGAGCTGTTCCGG TGGAATGTTTCGGACCTAGGTGGCCTGGGCTGTGGCCTGAAGAACAGGTCCTCAGAGGGCCC CAGCTCCCCTTCCGGGAAGCTCATGAGCCCCAAGCTGTATGTGTGGGCCAAAGACCGCCCTG AGATCTGGGAGGGAGAGCCTCCGTGTGTCCCACCGAGGGACAGCCTGAACCAGAGCCTCAGC CAGGACCTCACCATGGCCCCTGGCTCCACACTCTGGCTGTCCTGTGGGGTACCCCCTGACTC TGTGTCCAGGGGCCCCCTCTCCTGGACCCATGTGCACCCCAAGGGGCCTAAGTCATTGCTGA GCCTAGAGCTGAAGGACGATCGCCCGGCCAGAGATATGTGGGTAATGGAGACGGGTCTGTTG TTGCCCCGGGCCACAGCTCAAGACGCTGGAAAGTATTATTGTCACCGTGGCAACCTGACCAT GTCATTCCACCTGGAGATCACTGCTCGGCCAGTACTATGGCACTGGCTGCTGAGGACTGGTG GCTGGAAGGTCTCAGCTGTGACTTTGGCTTATCTGATCTTCTGCCTGTGTTCCCTTGTGGGC ATTCTTCATCTTCAAAGAGCCCTGGTCCTGAGGAGGAAAAGAAAGCGAATGACTGACCCCAC CAGGAGATTCTTCAAAGTGACGCCTCCCCCAGGAAGCGGGCCCCAGAACCAGTACGGGAACG TGCTGTCTCTCCCCACACCCACCTCAGGCCTCGGACGCGCCCAGCGTTGGGCCGCAGGCCTG GGGGGCACTGCCCCGTCTTATGGAAACCCGAGCAGCGACGTCCAGGCGGATGGAGCCTTGGG GTCCCGGAGCCCGCCGGGAGTGGGCCCAGAAGAAGAGGAAGGGGAGGGCTATGAGGAACCTG ACAGTGAGGAGGACTCCGAGTTCTATGAGAACGACTCCAACCTTGGGCAGGACCAGCTCTCC CAGGATGGCAGCGGCTACGAGAACCCTGAGGATGAGCCCCTGGGTCCTGAGGATGAAGACTC CTTCTCCAACGCTGAGTCTTATGAGAACGAGGATGAAGAGCTGACCCAGCCGGTCGCCAGGA CAATGGACTTCCTGAGCCCTCATGGGTCAGCCTGGGACCCCAGCCGGGAAGCAACCTCCCTG GGGTCCCAGTCCTATGAGGATATGAGAGGAATCCTGTATGCAGCCCCCCAGCTCCGCTCCAT TCGGGGCCAGCCTGGACCCAATCATGAGGAAGATGCAGACTCTTATGAGAACATGGATAATC CCGATGGGCCAGACCCAGCCTGGGGAGGAGGGGGCCGCATGGGCACCTGGAGCACCAGGTGA (SEQ ID NO: 569). An exemplary CD19 gene sequence is provided at ENSEMBL Accession No. ENSG00000177455 (SEQ ID NO: 570).

By “C/EBP Homologous Protein (CHOP) polypeptide” is meant a protein having at least about 85% amino acid sequence identity to GenBank Accession No. AAH03637.1, which is provided below, or a fragment thereof having immunomodulatory activity.

>AAH03637.1 DNA-damage-inducible transcript 3
[Homo sapiens]
(SEQ ID NO: 571)
MAAESLPFSFGTLSSWELEAWYEDLQEVLSSDENGGTYVSPPGNE
EEESKIFTTLDPASLAWLTEEEPEPAEVTSTSQSPHSPDSSQSSL
AQEEEEEDQGRTRKRKQSGHSPARAGKQRMKEKEQENERKVAQLA
EENERLKQEIERLTREVEATRRALIDRMVNLHQA.

By “C/EBP Homologous Protein (CHOP) polynucleotide” is meant a polynucleotide encoding a CHOP polypeptide, as well as the introns, exons, 3′ untranslated regions, 5′ untranslated regions, and regulatory sequences associated with its expression, or fragments thereof. In embodiments, a CHOP polynucleotide is the genomic sequence, cDNA, mRNA, or gene associated with and/or required for CHOP expression. An exemplary CHOP nucleic acid sequence is provided below (GenBank Accession No. BC003637.2:182-691).

>BC003637.2:182-691 Homo sapiens DNA-Damage-Inducible Transcript 3, mRNA (cDNA Clone MGC:4154 IMAGE:3530545), Complete Cds
ATGGCAGCTGAGTCATTGCCTTTCTCCTTTGGGACACTGTCCAGCTGGGAGCTGGAAGCCTG GTATGAGGACCTGCAAGAGGTCCTGTCTTCAGATGAAAATGGGGGTACCTATGTTTCACCTC CTGGAAATGAAGAGGAAGAATCAAAAATCTTCACCACTCTTGACCCTGCTTCTCTGGCTTGG CTGACTGAGGAGGAGCCAGAACCAGCAGAGGTCACAAGCACCTCCCAGAGCCCTCACTCTCC AGATTCCAGTCAGAGCTCCCTGGCTCAGGAGGAAGAGGAGGAAGACCAAGGGAGAACCAGGA AACGGAAACAGAGTGGTCATTCCCCAGCCCGGGCTGGAAAGCAGCGCATGAAGGAGAAAGAA CAGGAGAATGAAAGGAAAGTGGCACAGCTAGCTGAAGAGAATGAACGGCTCAAGCAGGAAAT CGAGCGCCTGACCAGGGAAGTAGAGGCGACTCGCCGAGCTCTGATTGACCGAATGGTGAATC TGCACCAAGCATGA (SEQ ID NO: 572). An exemplary CHOP gene sequence is provided at ENSEMBL Accession No. ENSG00000175197 (SEQ ID NO: 573).

By “class II, major histocompatibility complex, transactivator (CIITA) polypeptide” is meant a protein having at least about 85% amino acid sequence identity to NCBI Accession No. NP_001273331.1, which is provided below, or a fragment thereof having DNA binding activity.

>NP_001273331.1 MHC class II transactivator
isoform 1 [Homo sapiens]
(SEQ ID NO: 574)
MRCLAPRPAGSYLSEPQGSSQCATMELGPLEGGYLELLNSDADPL
CLYHFYDQMDLAGEEEIELYSEPDTDTINCDQFSRLLCDMEGDEE
TREAYANIAELDQYVFQDSQLEGLSKDIFIEHIGPDEVIGESMEM
PAEVGQKSQKRPFPEELPADLKHWKPAEPPTVVTGSLLVGPVSDC
STLPCLPLPALFNQEPASGQMRLEKTDQIPMPFSSSSLSCLNLPE
GPIQFVPTISTLPHGLWQISEAGTGVSSIFIYHGEVPQASQVPPP
SGFTVHGLPTSPDRPGSTSPFAPSATDLPSMPEPALTSRANMTEH
KTSPTQCPAAGEVSNKLPKWPEPVEQFYRSLQDTYGAEPAGPDGI
LVEVDLVQARLERSSSKSLERELATPDWAERQLAQGGLAEVLLAA
KEHRRPRETRVIAVLGKAGQGKSYWAGAVSRAWACGRLPQYDFVF
SVPCHCLNRPGDAYGLQDLLFSLGPQPLVAADEVFSHILKRPDRV
LLILDGFEELEAQDGFLHSTCGPAPAEPCSLRGLLAGLFQKKLLR
GCTLLLTARPRGRLVQSLSKADALFELSGFSMEQAQAYVMRYFES
SGMTEHQDRALTLLRDRPLLLSHSHSPTLCRAVCQLSEALLELGE
DAKLPSTLTGLYVGLLGRAALDSPPGALAELAKLAWELGRRHQST
LQEDQFPSADVRTWAMAKGLVQHPPRAAESELAFPSFLLQCFLGA
LWLALSGEIKDKELPQYLALTPRKKRPYDNWLEGVPRFLAGLIFQ
PPARCLGALLGPSAAASVDRKQKVLARYLKRLQPGTLRARQLLEL
LHCAHEAEEAGIWQHVVQELPGRLSFLGTRLTPPDAHVLGKALEA
AGQDFSLDLRSTGICPSGLGSLVGLSCVTRFRAALSDTVALWESL
QQHGETKLLQAAEEKFTIEPFKAKSLKDVEDLGKLVQTQRTRSSS
EDTAGELPAVRDLKKLEFALGPVSGPQAFPKLVRILTAFSSLQHL
DLDALSENKIGDEGVSQLSATFPQLKSLETLNLSQNNITDLGAYK
LAEALPSLAASLLRLSLYNNCICDVGAESLARVLPDMVSLRVMDV
QYNKFTAAGAQQLAASLRRCPHVETLAMWTPTIPFSVQEHLQQQD
SRISLR.

By “class II, major histocompatibility complex, transactivator (CIITA) polynucleotide” is meant a nucleic acid molecule encoding an CIITA polypeptide, as well as the introns, exons, 3′ untranslated regions, 5′ untranslated regions, and regulatory sequences associated with its expression, or fragments thereof. An exemplary CIITA polynucleotide is provided at NCBI Accession No. NM_001286402.1, which is provide below.

>NM_001286402.1 Homo sapiens Class II Major Histocompatibility Complex Transactivator (CIITA), Transcript Variant 1, mRNA
GGTTAGTGATGAGGCTAGTGATGAGGCTGTGTGCTTCTGAGCTGGGCATCCGAAGGCATCCT TGGGGAAGCTGAGGGCACGAGGAGGGGCTGCCAGACTCCGGGAGCTGCTGCCTGGCTGGGAT TCCTACACAATGCGTTGCCTGGCTCCACGCCCTGCTGGGTCCTACCTGTCAGAGCCCCAAGG CAGCTCACAGTGTGCCACCATGGAGTTGGGGCCCCTAGAAGGTGGCTACCTGGAGCTTCTTA ACAGCGATGCTGACCCCCTGTGCCTCTACCACTTCTATGACCAGATGGACCTGGCTGGAGAA GAAGAGATTGAGCTCTACTCAGAACCCGACACAGACACCATCAACTGCGACCAGTTCAGCAG GCTGTTGTGTGACATGGAAGGTGATGAAGAGACCAGGGAGGCTTATGCCAATATCGCGGAAC TGGACCAGTATGTCTTCCAGGACTCCCAGCTGGAGGGCCTGAGCAAGGACATTTTCATAGAG CACATAGGACCAGATGAAGTGATCGGTGAGAGTATGGAGATGCCAGCAGAAGTTGGGCAGAA AAGTCAGAAAAGACCCTTCCCAGAGGAGCTTCCGGCAGACCTGAAGCACTGGAAGCCAGCTG AGCCCCCCACTGTGGTGACTGGCAGTCTCCTAGTGGGACCAGTGAGCGACTGCTCCACCCTG CCCTGCCTGCCACTGCCTGCGCTGTTCAACCAGGAGCCAGCCTCCGGCCAGATGCGCCTGGA GAAAACCGACCAGATTCCCATGCCTTTCTCCAGTTCCTCGTTGAGCTGCCTGAATCTCCCTG AGGGACCCATCCAGTTTGTCCCCACCATCTCCACTCTGCCCCATGGGCTCTGGCAAATCTCT GAGGCTGGAACAGGGGTCTCCAGTATATTCATCTACCATGGTGAGGTGCCCCAGGCCAGCCA AGTACCCCCTCCCAGTGGATTCACTGTCCACGGCCTCCCAACATCTCCAGACCGGCCAGGCT CCACCAGCCCCTTCGCTCCATCAGCCACTGACCTGCCCAGCATGCCTGAACCTGCCCTGACC TCCCGAGCAAACATGACAGAGCACAAGACGTCCCCCACCCAATGCCCGGCAGCTGGAGAGGT CTCCAACAAGCTTCCAAAATGGCCTGAGCCGGTGGAGCAGTTCTACCGCTCACTGCAGGACA CGTATGGTGCCGAGCCCGCAGGCCCGGATGGCATCCTAGTGGAGGTGGATCTGGTGCAGGCC AGGCTGGAGAGGAGCAGCAGCAAGAGCCTGGAGCGGGAACTGGCCACCCCGGACTGGGCAGA ACGGCAGCTGGCCCAAGGAGGCCTGGCTGAGGTGCTGTTGGCTGCCAAGGAGCACCGGCGGC CGCGTGAGACACGAGTGATTGCTGTGCTGGGCAAAGCTGGTCAGGGCAAGAGCTATTGGGCT GGGGCAGTGAGCCGGGCCTGGGCTTGTGGCCGGCTTCCCCAGTACGACTTTGTCTTCTCTGT CCCCTGCCATTGCTTGAACCGTCCGGGGGATGCCTATGGCCTGCAGGATCTGCTCTTCTCCC TGGGCCCACAGCCACTCGTGGCGGCCGATGAGGTTTTCAGCCACATCTTGAAGAGACCTGAC CGCGTTCTGCTCATCCTAGACGGCTTCGAGGAGCTGGAAGCGCAAGATGGCTTCCTGCACAG CACGTGCGGACCGGCACCGGCGGAGCCCTGCTCCCTCCGGGGGCTGCTGGCCGGCCTTTTCC AGAAGAAGCTGCTCCGAGGTTGCACCCTCCTCCTCACAGCCCGGCCCCGGGGCCGCCTGGTC CAGAGCCTGAGCAAGGCCGACGCCCTATTTGAGCTGTCCGGCTTCTCCATGGAGCAGGCCCA GGCATACGTGATGCGCTACTTTGAGAGCTCAGGGATGACAGAGCACCAAGACAGAGCCCTGA CGCTCCTCCGGGACCGGCCACTTCTTCTCAGTCACAGCCACAGCCCTACTTTGTGCCGGGCA GTGTGCCAGCTCTCAGAGGCCCTGCTGGAGCTTGGGGAGGACGCCAAGCTGCCCTCCACGCT CACGGGACTCTATGTCGGCCTGCTGGGCCGTGCAGCCCTCGACAGCCCCCCCGGGGCCCTGG CAGAGCTGGCCAAGCTGGCCTGGGAGCTGGGCCGCAGACATCAAAGTACCCTACAGGAGGAC CAGTTCCCATCCGCAGACGTGAGGACCTGGGCGATGGCCAAAGGCTTAGTCCAACACCCACC GCGGGCCGCAGAGTCCGAGCTGGCCTTCCCCAGCTTCCTCCTGCAATGCTTCCTGGGGGCCC TGTGGCTGGCTCTGAGTGGCGAAATCAAGGACAAGGAGCTCCCGCAGTACCTAGCATTGACC CCAAGGAAGAAGAGGCCCTATGACAACTGGCTGGAGGGCGTGCCACGCTTTCTGGCTGGGCT GATCTTCCAGCCTCCCGCCCGCTGCCTGGGAGCCCTACTCGGGCCATCGGCGGCTGCCTCGG TGGACAGGAAGCAGAAGGTGCTTGCGAGGTACCTGAAGCGGCTGCAGCCGGGGACACTGCGG GCGCGGCAGCTGCTGGAGCTGCTGCACTGCGCCCACGAGGCCGAGGAGGCTGGAATTTGGCA GCACGTGGTACAGGAGCTCCCCGGCCGCCTCTCTTTTCTGGGCACCCGCCTCACGCCTCCTG ATGCACATGTACTGGGCAAGGCCTTGGAGGCGGCGGGCCAAGACTTCTCCCTGGACCTCCGC AGCACTGGCATTTGCCCCTCTGGATTGGGGAGCCTCGTGGGACTCAGCTGTGTCACCCGTTT CAGGGCTGCCTTGAGCGACACGGTGGCGCTGTGGGAGTCCCTGCAGCAGCATGGGGAGACCA AGCTACTTCAGGCAGCAGAGGAGAAGTTCACCATCGAGCCTTTCAAAGCCAAGTCCCTGAAG GATGTGGAAGACCTGGGAAAGCTTGTGCAGACTCAGAGGACGAGAAGTTCCTCGGAAGACAC AGCTGGGGAGCTCCCTGCTGTTCGGGACCTAAAGAAACTGGAGTTTGCGCTGGGCCCTGTCT CAGGCCCCCAGGCTTTCCCCAAACTGGTGCGGATCCTCACGGCCTTTTCCTCCCTGCAGCAT CTGGACCTGGATGCGCTGAGTGAGAACAAGATCGGGGACGAGGGTGTCTCGCAGCTCTCAGC CACCTTCCCCCAGCTGAAGTCCTTGGAAACCCTCAATCTGTCCCAGAACAACATCACTGACC TGGGTGCCTACAAACTCGCCGAGGCCCTGCCTTCGCTCGCTGCATCCCTGCTCAGGCTAAGC TTGTACAATAACTGCATCTGCGACGTGGGAGCCGAGAGCTTGGCTCGTGTGCTTCCGGACAT GGTGTCCCTCCGGGTGATGGACGTCCAGTACAACAAGTTCACGGCTGCCGGGGCCCAGCAGC TCGCTGCCAGCCTTCGGAGGTGTCCTCATGTGGAGACGCTGGCGATGTGGACGCCCACCATC CCATTCAGTGTCCAGGAACACCTGCAACAACAGGATTCACGGATCAGCCTGAGATGATCCCA GCTGTGCTCTGGACAGGCATGTTCTCTGAGGACACTAACCACGCTGGACCTTGAACTGGGTA CTTGTGGACACAGCTCTTCTCCAGGCTGTATCCCATGAGCCTCAGCATCCTGGCACCCGGCC CCTGCTGGTTCAGGGTTGGCCCCTGCCCGGCTGCGGAATGAACCACATCTTGCTCTGCTGAC AGACACAGGCCCGGCTCCAGGCTCCTTTAGCGCCCAGTTGGGTGGATGCCTGGTGGCAGCTG CGGTCCACCCAGGAGCCCCGAGGCCTTCTCTGAAGGACATTGCGGACAGCCACGGCCAGGCC AGAGGGAGTGACAGAGGCAGCCCCATTCTGCCTGCCCAGGCCCCTGCCACCCTGGGGAGAAA GTACTTCTTTTTTTTTATTTTTAGACAGAGTCTCACTGTTGCCCAGGCTGGCGTGCAGTGGT GCGATCTGGGTTCACTGCAACCTCCGCCTCTTGGGTTCAAGCGATTCTTCTGCTTCAGCCTC CCGAGTAGCTGGGACTACAGGCACCCACCATCATGTCTGGCTAATTTTTCATTTTTAGTAGA GACAGGGTTTTGCCATGTTGGCCAGGCTGGTCTCAAACTCTTGACCTCAGGTGATCCACCCA CCTCAGCCTCCCAAAGTGCTGGGATTACAAGCGTGAGCCACTGCACCGGGCCACAGAGAAAG TACTTCTCCACCCTGCTCTCCGACCAGACACCTTGACAGGGCACACCGGGCACTCAGAAGAC ACTGATGGGCAACCCCCAGCCTGCTAATTCCCCAGATTGCAACAGGCTGGGCTTCAGTGGCA GCTGCTTTTGTCTATGGGACTCAATGCACTGACATTGTTGGCCAAAGCCAAAGCTAGGCCTG GCCAGATGCACCAGCCCTTAGCAGGGAAACAGCTAATGGGACACTAATGGGGCGGTGAGAGG GGAACAGACTGGAAGCACAGCTTCATTTCCTGTGTCTTTTTTCACTACATTATAAATGTCTC TTTAATGTCACAGGCAGGTCCAGGGTTTGAGTTCATACCCTGTTACCATTTTGGGGTACCCA CTGCTCTGGTTATCTAATATGTAACAAGCCACCCCAAATCATAGTGGCTTAAAACAACACTC ACATTTA (SEQ ID NO: 575). An exemplary CIITA gene sequence is provided at (SEQ ID NO: 576).

By “Cytokine-inducible SH2-containing protein (CISH) polypeptide” is meant a protein having at least about 85% amino acid sequence identity to GenBank Accession No. AAD28471.2, which is provided below, or a fragment thereof having immunomodulatory activity.

>AAD28471.2 cytokine-inducible SH2-containing
protein [Homo sapiens]
(SEQ ID NO: 577)
MDTPLPRPRPLLAVERTGQRPLWAPSLELPKPVMQPLPAGAFLEE
VAEGTPAQTESEPKVLDPEEDLLCIAKTFSYLRESGWYWGSITAS
EARQHLQKMPEGTFLVRDSTHPSYLFTLSVKTTRGPTNVRIEYAD
SSFRLDSNCLSRPRILAFPDVVSLVQHYVASCTADTRSDSPDPAP
TPALPMPKEDAPSDPALPAPPPATAVHLKLVQPFVRRSSARSLQH
LCRLVINRLVADVDCLPLPRRMADYLRQYPFQL.

By “Cytokine-inducible SH2-containing protein (CISH) polynucleotide” is meant a polynucleotide encoding a CISH polypeptide, as well as the introns, exons, 3′ untranslated regions, 5′ untranslated regions, and regulatory sequences associated with its expression, or fragments thereof. In embodiments, a CISH polynucleotide is the genomic sequence, cDNA, mRNA, or gene associated with and/or required for CISH expression. An exemplary CISH nucleic acid sequence is provided below (GenBank Accession No. AF132297.2:280-1056).

>AF132297.2:280-1056 Homo sapiens Cytokine-Inducible SH2-Containing Protein (G18) mRNA, Complete Cds
ATGGACACACCCCTGCCCAGACCTCGTCCTTTGCTGGCTGTGGAGCGGACTGGGCAGCGGCC CCTGTGGGCCCCGTCCCTGGAACTGCCCAAGCCAGTCATGCAGCCCTTGCCTGCTGGGGCCT TCCTCGAGGAGGTGGCAGAGGGTACCCCAGCCCAGACAGAGAGTGAGCCAAAGGTGCTGGAC CCAGAGGAGGATCTGCTGTGCATAGCCAAGACCTTCTCCTACCTTCGGGAATCTGGCTGGTA TTGGGGTTCCATTACGGCCAGCGAGGCCCGACAACACCTGCAGAAGATGCCAGAAGGCACGT TCTTAGTACGTGACAGCACGCACCCCAGCTACCTGTTCACGCTGTCAGTGAAAACCACTCGT GGCCCCACCAATGTACGCATTGAGTATGCCGACTCCAGCTTCCGTCTGGACTCCAACTGCTT GTCCAGGCCACGCATCCTGGCCTTTCCGGATGTGGTCAGCCTTGTGCAGCACTATGTGGCCT CCTGCACTGCTGATACCCGAAGCGACAGCCCCGATCCTGCTCCCACCCCGGCCCTGCCTATG CCTAAGGAGGATGCGCCTAGTGACCCAGCACTGCCTGCTCCTCCACCAGCCACTGCTGTACA CCTAAAACTGGTGCAGCCCTTTGTACGCAGAAGCAGTGCCCGCAGCCTGCAACACCTGTGCC GCCTTGTCATCAACCGTCTGGTGGCCGACGTGGACTGCCTGCCACTGCCCCGGCGCATGGCC GACTACCTCCGACAGTACCCCTTCCAGCTCTGA (SEQ ID NO: 578). An exemplary CISH gene sequence is provided at ENSEMBL Accession No. ENSG00000114737 (SEQ ID NO: 579).

By “Deoxycytidine kinase (DCK) polypeptide” is meant a protein having at least about 85% amino acid sequence identity to GenBank Accession No. AAA35752.1, which is provided below, or a fragment thereof having immunomodulatory activity.

>AAA35752.1 deoxycytidine kinase [Homo sapiens]
(SEQ ID NO: 580)
MATPPKRSCPSFSASSEGTRIKKISIEGNIAAGKSTFVNILKQLC
EDWEVVPEPVARWCNVQSTQDEFEELTMSQKNGGNVLQMMYEKPE
RWSFTFQTYACLSRIRAQLASLNGKLKDAEKPVLFFERSVYSDRY
IFASNLYESECMNETEWTIYQDWHDWMNNQFGQSLELDGIIYLQA
TPETCLHRIYLRGRNEEQGIPLEYLEKLHYKHESWLLHRTLKTNF
DYLQEVPILTLDVNEDFKDKYESLVEKVKEFLSTL.

By “Deoxycytidine kinase (DCK) polynucleotide” is meant a polynucleotide encoding a DCK polypeptide, as well as the introns, exons, 3′ untranslated regions, 5′ untranslated regions, and regulatory sequences associated with its expression, or fragments thereof. In embodiments, a DCK polynucleotide is the genomic sequence, cDNA, mRNA, or gene associated with and/or required for DCK expression. An exemplary DCK nucleic acid sequence is provided below (GenBank Accession No. M60527.1:160-942).

>M60527.1:160-942 Human Deoxycytidine Kinase mRNA, Complete Cds
ATGGCCACCCCGCCCAAGAGAAGCTGCCCGTCTTTCTCAGCCAGCTCTGAGGGGACCCGCAT CAAGAAAATCTCCATCGAAGGGAACATCGCTGCAGGGAAGTCAACATTTGTGAATATCCTTA AACAATTGTGTGAAGATTGGGAAGTGGTTCCTGAACCTGTTGCCAGATGGTGCAATGTTCAA AGTACTCAAGATGAATTTGAGGAACTTACAATGTCTCAGAAAAATGGTGGGAATGTTCTTCA GATGATGTATGAGAAACCTGAACGATGGTCTTTTACCTTCCAAACATATGCCTGTCTCAGTC GAATAAGAGCTCAGCTTGCCTCTCTGAATGGCAAGCTCAAAGATGCAGAGAAACCTGTATTA TTTTTTGAACGATCTGTGTATAGTGACAGGTATATTTTTGCATCTAATTTGTATGAATCTGA ATGCATGAATGAGACAGAGTGGACAATTTATCAAGACTGGCATGACTGGATGAATAACCAAT TTGGCCAAAGCCTTGAATTGGATGGAATCATTTATCTTCAAGCCACTCCAGAGACATGCTTA CATAGAATATATTTACGGGGAAGAAATGAAGAGCAAGGCATTCCTCTTGAATATTTAGAGAA GCTTCATTATAAACATGAAAGCTGGCTCCTGCATAGGACACTGAAAACCAACTTCGATTATC TTCAAGAGGTGCCTATCTTAACACTGGATGTTAATGAAGACTTTAAAGACAAATATGAAAGT CTGGTTGAAAAGGTCAAAGAGTTTTTGAGTACTTTGTGA (SEQ ID NO: 581). An exemplary DCK gene sequence is provided at ENSEMBL Accession No. ENSG00000156136 (SEQ ID NO: 582).

By “Diacylglycerol kinase alpha/zeta (DGKα; DGKa) polypeptide” is meant a protein having at least about 85% amino acid sequence identity to GenBank Accession No. AAC34805.1, which is provided below, or fragments thereof having immunomodulatory activity.

>AAC34805.1 diacylglycerol kinase
alpha [Homo sapiens]
(SEQ ID NO: 583)
MAKERGLISPSDFAQLQKYMEYSTKKVSDVLKLFEDGEMAKYVQG
DAIGYEGFQQFLEIYLEVDNVPRHLSLALFQSFETGHCLNETNVT
KGMVKQMWCVSMMFPATFPFWRVVGQKTS.

By “Diacylglycerol kinase alpha/zeta (DGKα; DGKa) polynucleotide” is meant a polynucleotide encoding a DGKα polypeptide, as well as the introns, exons, 3′ untranslated regions, 5′ untranslated regions, and regulatory sequences associated with its expression, or fragments thereof. In embodiments, a DGKα polynucleotide is the genomic sequence, cDNA, mRNA, or gene associated with and/or required for DGKα/ζ expression. An exemplary DGKα nucleic acid sequences is provided below (GenBank Accession No. AF064770.1:240-599).

>AF064770.1:240-599 Homo sapiens Clone 17 Diacylglycerol Kinase Alpha (DAGK1) mRNA, Complete Cds
ATGGCCAAGGAGAGGGGCCTAATAAGCCCCAGTGATTTTGCCCAGCTGCAAAAATACATGGA ATACTCCACCAAAAAGGTCAGTGATGTCCTAAAGCTCTTCGAGGATGGCGAGATGGCTAAAT ATGTCCAAGGAGATGCCATTGGGTACGAGGGATTCCAGCAATTCCTGGAAATCTATCTCGAA GTGGATAATGTTCCCAGACACCTAAGCCTGGCACTGTTTCAATCCTTTGAGACTGGTCACTG CTTAAATGAGACAAATGTGACAAAAGGTATGGTCAAGCAGATGTGGTGTGTCTCAATGATGT TTCCTGCTACTTTTCCCTTCTGGAGGGTGGTCGGCCAGAAGACAAGTTAG (SEQ ID NO: 584). An exemplary DGK alpha gene sequence is provided at ENSEMBL Accession No. ENSG00000065357 (SEQ ID NO: 585).

By “Diacylglycerol kinase alpha/zeta (DGKζ; DGKz) polypeptide” is meant a protein having at least about 85% amino acid sequence identity to GenBank Accession No. AAC50478.1 which is provided below, or fragments thereof having immunomodulatory activity.

>AAC50478.1 diacylglycerol kinase
zeta [Homo sapiens]
(SEQ ID NO: 586)
MEPRDGSPEARSSDSESASASSSGSERDAGPEPDKAPRRLNKRRF
PGLRLFGHRKAITKSGLQHLAPPPPTPGAPCSESERQIRSTVDWS
ESATYGEHIWFETNVSGDFCYVGEQYCVARMLKSVSRRKCAACKI
VVHTPCIEQLEKINFRCKPSFRESGSRNVREPTFVRHHWVHRRRQ
DGKCRHCGKGFQQKFTFHSKEIVAISCSWCKQAYHSKVSCFMLQQ
IEEPCSLGVHAAVVIPPTWILRARRPQNTLKASKKKKRASFKRKS
SKKGPEEGRWRPFIIRPTPSPLMKPLLVFVNPKSGGNQGAKIIQS
FLWYLNPRQVFDLSQGGPKEALEMYRKVHNLRILACGGDGTVGWI
LSTLDQLRLKPPPPVAILPLGTGNDLARTLNWGGGYTDEPVSKIL
SHVEEGNVVQLDRWDLHAEPNPEAGPEDRDEGATDRLPLDVFNNY
FSLGFDAHVTLEFHESREANPEKFNSRFRNKMFYAGTAFSDFLMG
SSKDLAKHIRVVCDGMDLTPKIQDLKPQCVVFLNIPRYCAGTMPW
GHPGEHHDFEPQRHDDGYLEVIGFTMTSLAALQVGGHGERLTQCR
EVVLTTSKAIPVQVDGEPCKLAASRIRIALRNQATMVQKAKRRSA
APLHSDQQPVPEQLRIQVSRVSMHDYEALHYDKEQLKEASVPLGT
VVVPGDSDLELCRAHIERLQQEPDGAGAKSPTCQKLSPKWCFLDA
TTASRFYRIDRAQEHLNYVTEIAQDEIYILDPELLGASARPDLPT
PTSPLPTSPCSPTPRSLQGDAAPPQGEELIEAAKRNDFCKLQELH
RAGGDLMHRDEQSRTLLHHAVSTGSKDVVRYLLDHAPPEILDAVE
ENGETCLHQAAALGQRTICHYIVEAGASLMKTDQQGDTPRQRAEK
AQDTELAAYLENRQHYQMIQREDQETAV.

By “Diacylglycerol kinase alpha/zeta (DGKζ; DGKz) polynucleotide” is meant a polynucleotide encoding a DGKζ polypeptide, as well as the introns, exons, 3′ untranslated regions, 5′ untranslated regions, and regulatory sequences associated with its expression, or fragments thereof. In embodiments, a DGK polynucleotide is the genomic sequence, cDNA, mRNA, or gene associated with and/or required for DGKζ expression. An exemplary DGK nucleic acid sequence is provided below (GenBank Accession No. U51477.1:89-2875).

>U51477.1:89-2875 Human Diacylglycerol Kinase Zeta mRNA, Complete Cds
ATGGAGCCGCGGGACGGTAGCCCCGAGGCCCGGAGCAGCGACTCCGAGTCGGCTTCCGCCTC GTCCAGCGGCTCCGAGCGCGACGCCGGTCCCGAGCCGGACAAGGCGCCGCGGCGACTCAACA AGCGGCGCTTCCCGGGGCTGCGGCTCTTCGGGCACAGGAAAGCCATCACCAAGTCGGGCCTC CAGCACCTGGCCCCCCCTCCGCCCACCCCTGGGGCCCCGTGCAGCGAGTCAGAGCGGCAGAT CCGGAGTACAGTGGACTGGAGCGAGTCAGCGACATATGGGGAGCACATCTGGTTCGAGACCA ACGTGTCCGGGGACTTCTGCTACGTTGGGGAGCAGTACTGTGTAGCCAGGATGCTGAAGTCA GTGTCTCGAAGAAAGTGCGCAGCCTGCAAGATTGTGGTGCACACGCCCTGCATCGAGCAGCT GGAGAAGATAAATTTCCGCTGTAAGCCGTCCTTCCGTGAATCAGGCTCCAGGAATGTCCGCG AGCCAACCTTTGTACGGCACCACTGGGTACACAGACGACGCCAGGACGGCAAGTGTCGGCAC TGTGGGAAGGGATTCCAGCAGAAGTTCACCTTCCACAGCAAGGAGATTGTGGCCATCAGCTG CTCGTGGTGCAAGCAGGCATACCACAGCAAGGTGTCCTGCTTCATGCTGCAGCAGATCGAGG AGCCGTGCTCGCTGGGGGTCCACGCAGCCGTGGTCATCCCGCCCACCTGGATCCTCCGCGCC CGGAGGCCCCAGAATACTCTGAAAGCAAGCAAGAAGAAGAAGAGGGCATCCTTCAAGAGGAA GTCCAGCAAGAAAGGGCCTGAGGAGGGCCGCTGGAGACCCTTCATCATCAGGCCCACCCCCT CCCCGCTCATGAAGCCCCTGCTGGTGTTTGTGAACCCCAAGAGTGGGGGCAACCAGGGTGCA AAGATCATCCAGTCTTTCCTCTGGTATCTCAATCCCCGACAAGTCTTCGACCTGAGCCAGGG AGGGCCCAAGGAGGCGCTGGAGATGTACCGCAAAGTGCACAACCTGCGGATCCTGGCGTGCG GGGGCGACGGCACGGTGGGCTGGATCCTCTCCACCCTGGACCAGCTACGCCTGAAGCCGCCA CCCCCTGTTGCCATCCTGCCCCTGGGTACTGGCAACGACTTGGCCCGAACCCTCAACTGGGG TGGGGGCTACACAGATGAGCCTGTGTCCAAGATCCTCTCCCACGTGGAGGAGGGGAACGTGG TACAGCTGGACCGCTGGGACCTCCACGCTGAGCCCAACCCCGAGGCAGGGCCTGAGGACCGA GATGAAGGCGCCACCGACCGGTTGCCCCTGGATGTCTTCAACAACTACTTCAGCCTGGGCTT TGACGCCCACGTCACCCTGGAGTTCCACGAGTCTCGAGAGGCCAACCCAGAGAAATTCAACA GCCGCTTTCGGAATAAGATGTTCTACGCCGGGACAGCTTTCTCTGACTTCCTGATGGGCAGC TCCAAGGACCTGGCCAAGCACATCCGAGTGGTGTGTGATGGAATGGACTTGACTCCCAAGAT CCAGGACCTGAAACCCCAGTGTGTTGTTTTCCTGAACATCCCCAGGTACTGTGCGGGCACCA TGCCCTGGGGCCACCCTGGGGAGCACCACGACTTTGAGCCCCAGCGGCATGACGACGGCTAC CTCGAGGTCATTGGCTTCACCATGACGTCGTTGGCCGCGCTGCAGGTGGGCGGACACGGCGA GCGGCTGACGCAGTGTCGCGAGGTGGTGCTCACCACATCCAAGGCCATCCCGGTGCAGGTGG ATGGCGAGCCCTGCAAGCTTGCAGCCTCACGCATCCGCATCGCCCTGCGCAACCAGGCCACC ATGGTGCAGAAGGCCAAGCGGCGGAGCGCCGCCCCCCTGCACAGCGACCAGCAGCCGGTGCC AGAGCAGTTGCGCATCCAGGTGAGTCGCGTCAGCATGCACGACTATGAGGCCCTGCACTACG ACAAGGAGCAGCTCAAGGAGGCCTCTGTGCCGCTGGGCACTGTGGTGGTCCCAGGAGACAGT GACCTAGAGCTCTGCCGTGCCCACATTGAGAGACTCCAGCAGGAGCCCGATGGTGCTGGAGC CAAGTCCCCGACATGCCAGAAACTGTCCCCCAAGTGGTGCTTCCTGGACGCCACCACTGCCA GCCGCTTCTACAGGATCGACCGAGCCCAGGAGCACCTCAACTATGTGACTGAGATCGCACAG GATGAGATTTATATCCTGGACCCTGAGCTGCTGGGGGCATCGGCCCGGCCTGACCTCCCAAC CCCCACTTCCCCTCTCCCCACCTCACCCTGCTCACCCACGCCCCGGTCACTGCAAGGGGATG CTGCACCCCCTCAAGGTGAAGAGCTGATTGAGGCTGCCAAGAGGAACGACTTCTGTAAGCTC CAGGAGCTGCACCGAGCTGGGGGCGACCTCATGCACCGAGACGAGCAGAGTCGCACGCTCCT GCACCACGCAGTCAGCACTGGCAGCAAGGATGTGGTCCGCTACCTGCTGGACCACGCCCCCC CAGAGATCCTTGATGCGGTGGAGGAAAACGGGGAGACCTGTTTGCACCAAGCAGCGGCCCTG GGCCAGCGCACCATCTGCCACTACATCGTGGAGGCCGGGGCCTCGCTCATGAAGACAGACCA GCAGGGCGACACTCCCCGGCAGCGGGCTGAGAAGGCTCAGGACACCGAGCTGGCCGCCTACC TGGAGAACCGGCAGCACTACCAGATGATCCAGCGGGAGGACCAGGAGACGGCTGTGTAG (SEQ ID NO: 587). An exemplary DGK zeta gene sequence is provided at ENSEMBL Accession No. ENSG00000149091 (SEQ ID NO: 588).

By “DEAH-box helicase 37 (DHX37) polypeptide” is meant a protein having at least about 85% amino acid sequence identity to GenBank Accession No. AAH37964.1, which is provided below, or a fragment thereof having immunomodulatory activity.

>AAH37964.1 DEAH (Asp-Glu-Ala-His) box
polypeptide 37 [Homo sapiens]
(SEQ ID NO: 589)
MGKLRRRYNIKGRQQAGPGPSKGPPEPPPVQLELEDKDTLKGVDA
SNALVLPGKKKKKTKAPPLSKKEKKPLTKKEKKVLQKILEQKEKK
SQRAEMLQKLSEVQASEAEMRLFYTTSKLGTGNRMYHTKEKADEV
VAPGQEKISSLSGAHRKRRRWPSAEEEEEEEEESESELEEESELD
EDPAAEPAEAGVGTTVAPLPPAPAPSSQPVPAGMTVPPPPAAAPP
LPRALAKPAVFIPVNRSPEMQEERLKLPILSEEQVIMEAVAEHPI
VIVCGETGSGKTTQVPQFLYEAGFSSEDSIIGVTEPRRVAAVAMS
QRVAKEMNLSQRVVSYQIRYEGNVTEETRIKFMTDGVLLKEIQKD
FLLLRYKVVIIDEAHERSVYTDILIGLLSRIVTLRAKRNLPLKLL
IMSATLRVEDFTQNPRLFAKPPPVIKVESRQFPVTVHFNKRTPLE
DYSGECFRKVCKIHRMLPAGGILVFLTGQAEVHALCRRLRKAFPP
SRARPQEKDDDQKDSVEEMRKFKKSRARAKKARAEVLPQINLDHY
SVLPAGEGDEDREAEVDEEEGALDSDLDLDLGDGGQDGGEQPDAS
LPLHVLPLYSLLAPEKQAQVFKPPPEGTRLCVVATNVAETSLTIP
GIKYVVDCGKVKKRYYDRVTGVSSFRVTWVSQASADQRAGRAGRT
EPGHCYRLYSSAVFGDFEQFPPPEITRRPVEDLILQMKALNVEKV
INFPFPTPPSVEALLAAEELLIALGALQPPQKAERVKQLQENRLS
CPITALGRTMATFPVAPRYAKMLALSRQHGCLPYAITIVASMTVR
ELFEELDRPAASDEELTRLKSKRARVAQMKRTWAGQGASLKLGDL
MVLLGAVGACEYASCTPQFCEANGLRYKAMMEIRRLRGQLTTAVN
AVCPEAELFVDPKMQPPTESQVTYLRQIVTAGLGDHLARRVQSEE
MLEDKWRNAYKTPLLDDPVFIHPSSVLFKELPEFVVYQEIVETTK
MYMKGVSSVEVQWIPALLPSYCQFDKPLEEPAPTYCPERGRVLCH
RASVFYRVGWPLPAIEVDFPEGIDRYKHFARFLLEGQVFRKLASY
RSCLLSSPGTMLKTWARLQPRTESLLRALVAEKADCHEALLAAWK
KNPKYLLAEYCEWLPQAMHPDIEKAWPPTTVH.

By “DEAH-box helicase 37 (DHX37) polynucleotide” is meant a polynucleotide encoding a DHX37 polypeptide, as well as the introns, exons, 3′ untranslated regions, 5′ untranslated regions, and regulatory sequences associated with its expression, or fragments thereof. In embodiments, a DHX37 polynucleotide is the genomic sequence, cDNA, mRNA, or gene associated with and/or required for DHX37 expression. An exemplary DHX37 nucleic acid sequence is provided below (GenBank Accession No. BC037964.1:82-3555).

>BC037964.1:82-3555 Homo sapiens DEAH (Asp-Glu-Ala-His) Box Polypeptide 37, mRNA (cDNA Clone MGC:46245 IMAGE:5736582), Complete Cds
ATGGGGAAGCTGCGCCGGCGCTACAACATCAAGGGGCGCCAGCAGGCGGGCCCCGGACCCTC GAAGGGCCCCCCCGAGCCGCCCCCCGTGCAGCTGGAACTGGAGGACAAGGACACGTTGAAGG GAGTTGATGCAAGCAACGCGCTCGTTCTACCGGGGAAGAAGAAAAAGAAGACCAAAGCCCCT CCCCTGTCGAAGAAGGAGAAGAAGCCTCTGACCAAGAAGGAGAAGAAAGTGCTGCAGAAAAT CTTAGAACAGAAGGAGAAAAAGAGCCAGCGAGCAGAGATGCTACAGAAGCTGAGTGAAGTCC AGGCTTCCGAAGCTGAGATGAGACTCTTTTATACCACTTCCAAGCTAGGCACTGGGAACCGC ATGTATCACACCAAAGAGAAGGCTGACGAGGTGGTAGCCCCGGGCCAGGAGAAGATCAGTAG CCTCAGCGGTGCCCACCGGAAGCGTCGCCGCTGGCCCTCAGCTGAGGAGGAGGAGGAGGAGG AGGAGGAGTCGGAATCGGAGCTGGAGGAGGAGTCGGAGCTGGACGAGGACCCAGCTGCTGAG CCGGCTGAGGCTGGTGTGGGGACCACCGTGGCACCTCTGCCGCCAGCTCCAGCACCCAGCAG TCAGCCCGTGCCGGCTGGGATGACTGTTCCTCCTCCTCCAGCTGCAGCCCCACCACTGCCCA GGGCCCTGGCTAAGCCCGCCGTCTTCATCCCCGTGAACCGCTCCCCGGAAATGCAGGAGGAA CGGCTGAAGCTCCCAATTCTCTCCGAAGAACAAGTAATCATGGAGGCTGTGGCCGAGCACCC CATCGTCATCGTGTGTGGTGAGACCGGCAGCGGGAAGACCACACAGGTGCCTCAGTTTCTCT ATGAAGCAGGCTTCAGCAGTGAAGACAGCATCATCGGTGTCACGGAGCCCCGCCGAGTGGCC GCCGTGGCCATGTCCCAGCGAGTGGCCAAGGAGATGAATCTGTCCCAGCGGGTCGTCTCCTA CCAGATCCGGTATGAAGGAAACGTGACAGAGGAGACCAGAATCAAGTTCATGACGGATGGTG TGCTGCTTAAAGAAATCCAGAAGGACTTCCTGCTGCTGCGGTACAAGGTGGTGATCATCGAC GAGGCCCACGAGAGGAGCGTGTACACGGACATCCTCATCGGCCTCCTGTCCCGCATTGTGAC TCTCCGGGCTAAGAGGAACCTGCCACTCAAGCTGCTCATCATGTCGGCCACGCTGCGGGTGG AGGACTTCACCCAGAACCCACGGCTCTTCGCCAAGCCGCCGCCGGTCATCAAGGTGGAATCC AGGCAGTTCCCAGTGACTGTGCATTTCAACAAGCGGACACCGCTGGAAGACTACAGTGGCGA GTGCTTCCGGAAGGTCTGCAAGATCCACCGGATGCTGCCCGCAGGTGGCATCCTGGTGTTCC TGACGGGGCAGGCTGAGGTGCATGCGCTGTGCCGCAGGCTCAGGAAGGCTTTCCCACCCTCC AGAGCCCGGCCACAAGAAAAGGACGACGATCAGAAAGACTCGGTGGAGGAAATGCGGAAGTT TAAGAAGTCAAGGGCCAGGGCCAAGAAGGCGCGGGCTGAGGTGCTGCCCCAGATCAACTTGG ATCATTACTCGGTGTTACCGGCAGGCGAAGGCGATGAGGACAGGGAGGCAGAAGTGGATGAG GAAGAGGGGGCCCTGGACTCCGACCTCGATCTGGACCTGGGGGATGGCGGGCAAGATGGAGG TGAGCAGCCGGATGCCTCCCTCCCGCTCCACGTGCTCCCGCTGTACTCTCTGCTGGCCCCAG AGAAGCAAGCACAGGTCTTTAAGCCTCCACCGGAGGGGACTCGGTTGTGTGTTGTGGCCACC AATGTGGCCGAGACGTCGCTTACCATCCCTGGCATCAAGTACGTGGTGGACTGTGGGAAGGT CAAGAAACGCTACTACGACCGCGTCACTGGCGTATCCTCCTTCCGTGTCACCTGGGTCTCCC AGGCATCAGCTGACCAGCGAGCGGGCAGAGCAGGACGGACGGAGCCCGGCCACTGCTACAGG CTGTATTCATCTGCGGTTTTTGGTGACTTCGAGCAGTTTCCTCCTCCAGAAATCACCCGGAG GCCTGTTGAAGACTTAATCCTTCAAATGAAGGCGCTCAACGTTGAAAAGGTCATCAACTTCC CCTTCCCGACGCCCCCCTCCGTGGAAGCCCTTCTTGCCGCCGAGGAGCTGTTGATCGCACTG GGTGCCCTGCAACCGCCCCAGAAAGCAGAAAGGGTGAAGCAACTGCAGGAGAACCGGCTGAG CTGCCCCATCACTGCGCTGGGCCGGACAATGGCCACGTTCCCCGTGGCACCCCGCTACGCTA AGATGCTGGCACTGAGCCGACAACACGGCTGCCTGCCCTATGCCATCACCATCGTGGCCAGC ATGACGGTGCGGGAGCTGTTTGAGGAGCTGGACAGACCAGCGGCCAGTGACGAGGAGCTCAC CAGGCTGAAGAGCAAGCGTGCCCGGGTGGCCCAGATGAAGAGGACCTGGGCAGGGCAGGGGG CTTCTCTGAAGCTCGGCGACCTCATGGTGCTGCTGGGCGCCGTGGGAGCCTGTGAGTATGCC AGCTGCACACCCCAGTTTTGCGAAGCCAACGGGCTGCGGTACAAAGCCATGATGGAGATCCG GCGCCTGCGGGGCCAGCTGACCACCGCAGTCAATGCCGTGTGCCCCGAGGCTGAGCTCTTCG TGGATCCCAAGATGCAGCCGCCCACCGAGAGCCAGGTGACCTACCTGCGACAGATCGTGACG GCAGGCCTGGGGGACCACTTGGCCCGCAGGGTCCAGAGCGAGGAGATGCTGGAGGACAAGTG GAGGAACGCCTACAAGACCCCTCTCCTCGACGACCCTGTCTTCATCCACCCCAGCTCCGTCC TTTTCAAAGAGCTCCCCGAGTTTGTGGTCTACCAGGAAATCGTGGAGACCACTAAGATGTAC ATGAAAGGCGTCTCTAGCGTGGAGGTCCAGTGGATCCCGGCCCTGCTGCCCTCTTACTGCCA GTTTGACAAGCCCCTGGAGGAACCAGCCCCTACATACTGCCCCGAGCGGGGGCGGGTGCTGT GTCACCGGGCCAGCGTGTTCTATCGCGTGGGCTGGCCGCTCCCCGCCATCGAGGTGGATTTT CCAGAGGGGATTGACCGCTACAAGCACTTTGCCCGGTTCCTGCTGGAAGGGCAGGTCTTCCG CAAGCTGGCCTCATACCGGAGCTGTCTGCTGTCCAGCCCCGGCACCATGCTGAAGACGTGGG CCAGGCTGCAGCCCCGTACGGAGAGCCTTCTGCGAGCCCTGGTTGCAGAGAAGGCTGACTGC CATGAAGCCTTGCTGGCTGCTTGGAAGAAAAACCCCAAATACCTGCTGGCTGAGTACTGTGA GTGGCTTCCACAGGCCATGCACCCCGATATCGAGAAAGCCTGGCCCCCCACCACTGTCCACT GA (SEQ ID NO: 590). An exemplary DHX37 gene sequence is provided at ENSEMBL Accession No. ENSG00000150990 (SEQ ID NO: 591).

By “DNA methyltransferase 3A (DNMT3A) polypeptide” is meant a protein having at least about 85% amino acid sequence identity to GenBank Accession No. AAD33084.2, which is provided below, or a fragment thereof having immunomodulatory activity.

>AAD33084.2 DNA cytosine methyltransferase
3 alpha [Homo sapiens]
(SEQ ID NO: 592)
MPAMPSSGPGDTSSSAAEREEDRKDGEEQEEPRGKEERQEPSTTA
RKVGRPGRKRKHPPVESGDTPKDPAVISKSPSMAQDSGASELLPN
GDLEKRSEPQPEEGSPAGGQKGGAPAEGEGAAETLPEASRAVENG
CCTPKEGRGAPAEAGKEQKETNIESMKMEGSRGRLRGGLGWESSL
RQRPMPRLTFQAGDPYYISKRKRDEWLARWKREAEKKAKVIAGMN
AVEENQGPGESQKVEEASPPAVQQPTDPASPTVATTPEPVGSDAG
DKNATKAGDDEPEYEDGRGFGIGELVWGKLRGFSWWPGRIVSWWM
TGRSRAAEGTRWVMWFGDGKFSVVCVEKLMPLSSFCSAFHQATYN
KQPMYRKAIYEVLQVASSRAGKLFPVCHDSDESDTAKAVEVQNKP
MIEWALGGFQPSGPKGLEPPEEEKNPYKEVYTDMWVEPEAAAYAP
PPPAKKPRKSTAEKPKVKEIIDERTRERLVYEVRQKCRNIEDICI
SCGSLNVTLEHPLFVGGMCQNCKNCFLECAYQYDDDGYQSYCTIC
CGGREVLMCGNNNCCRCFCVECVDLLVGPGAAQAAIKEDPWNCYM
CGHKGTYGLLRRREDWPSRLQMFFANNHDQEFDPPKVYPPVPAEK
RKPIRVLSLFDGIATGLLVLKDLGIQVDRYIASEVCEDSITVGMV
RHQGKIMYVGDVRSVTQKHIQEWGPFDLVIGGSPCNDLSIVNPAR
KGLYEGTGRLFFEFYRLLHDARPKEGDDRPFFWLFENVVAMGVSD
KRDISRFLESNPVMIDAKEVSAAHRARYFWGNLPGMNRPLASTVN
DKLELQECLEHGRIAKFSKVRTITTRSNSIKQGKDQHFPVFMNEK
EDILWCTEMERVFGFPVHYTDVSNMSRLARQRLLGRSWSVPVIRH
LFAPLKEYFACV.

By “DNA methyltransferase 3A (DNMT3A) polynucleotide” is meant a polynucleotide encoding a DNMT3A polypeptide, as well as the introns, exons, 3′ untranslated regions, 5′ untranslated regions, and regulatory sequences associated with its expression, or fragments thereof. In embodiments, a DNMT3A polynucleotide is the genomic sequence, cDNA, mRNA, or gene associated with and/or required for DNMT3A expression. An exemplary DNMT3A nucleic acid sequence is provided below (GenBank Accession No. AF067972.2:230-2968).

>AF067972.2:230-2968 Homo sapiens DNA Cytosine Methyltransferase 3 Alpha (DNMT3A) mRNA, Complete Cds
ATGCCCGCCATGCCCTCCAGCGGCCCCGGGGACACCAGCAGCTCTGCTGCGGAGCGGGAGGA GGACCGAAAGGACGGAGAGGAGCAGGAGGAGCCGCGTGGCAAGGAGGAGCGCCAAGAGCCCA GCACCACGGCACGGAAGGTGGGGCGGCCTGGGAGGAAGCGCAAGCACCCCCCGGTGGAAAGC GGTGACACGCCAAAGGACCCTGCGGTGATCTCCAAGTCCCCATCCATGGCCCAGGACTCAGG CGCCTCAGAGCTATTACCCAATGGGGACTTGGAGAAGCGGAGTGAGCCCCAGCCAGAGGAGG GGAGCCCTGCTGGGGGGCAGAAGGGCGGGGCCCCAGCAGAGGGAGAGGGTGCAGCTGAGACC CTGCCTGAAGCCTCAAGAGCAGTGGAAAATGGCTGCTGCACCCCCAAGGAGGGCCGAGGAGC CCCTGCAGAAGCGGGCAAAGAACAGAAGGAGACCAACATCGAATCCATGAAAATGGAGGGCT CCCGGGGCCGGCTGCGGGGTGGCTTGGGCTGGGAGTCCAGCCTCCGTCAGCGGCCCATGCCG AGGCTCACCTTCCAGGCGGGGGACCCCTACTACATCAGCAAGCGCAAGCGGGACGAGTGGCT GGCACGCTGGAAAAGGGAGGCTGAGAAGAAAGCCAAGGTCATTGCAGGAATGAATGCTGTGG AAGAAAACCAGGGGCCCGGGGAGTCTCAGAAGGTGGAGGAGGCCAGCCCTCCTGCTGTGCAG CAGCCCACTGACCCCGCATCCCCCACTGTGGCTACCACGCCTGAGCCCGTGGGGTCCGATGC TGGGGACAAGAATGCCACCAAAGCAGGCGATGACGAGCCAGAGTACGAGGACGGCCGGGGCT TTGGCATTGGGGAGCTGGTGTGGGGGAAACTGCGGGGCTTCTCCTGGTGGCCAGGCCGCATT GTGTCTTGGTGGATGACGGGCCGGAGCCGAGCAGCTGAAGGCACCCGCTGGGTCATGTGGTT CGGAGACGGCAAATTCTCAGTGGTGTGTGTTGAGAAGCTGATGCCGCTGAGCTCGTTTTGCA GTGCGTTCCACCAGGCCACGTACAACAAGCAGCCCATGTACCGCAAAGCCATCTACGAGGTC CTGCAGGTGGCCAGCAGCCGCGCGGGGAAGCTGTTCCCGGTGTGCCACGACAGCGATGAGAG TGACACTGCCAAGGCCGTGGAGGTGCAGAACAAGCCCATGATTGAATGGGCCCTGGGGGGCT TCCAGCCTTCTGGCCCTAAGGGCCTGGAGCCACCAGAAGAAGAGAAGAATCCCTACAAAGAA GTGTACACGGACATGTGGGTGGAACCTGAGGCAGCTGCCTACGCACCACCTCCACCAGCCAA AAAGCCCCGGAAGAGCACAGCGGAGAAGCCCAAGGTCAAGGAGATTATTGATGAGCGCACAA GAGAGCGGCTGGTGTACGAGGTGCGGCAGAAGTGCCGGAACATTGAGGACATCTGCATCTCC TGTGGGAGCCTCAATGTTACCCTGGAACACCCCCTCTTCGTTGGAGGAATGTGCCAAAACTG CAAGAACTGCTTTCTGGAGTGTGCGTACCAGTACGACGACGACGGCTACCAGTCCTACTGCA CCATCTGCTGTGGGGGCCGTGAGGTGCTCATGTGCGGAAACAACAACTGCTGCAGGTGCTTT TGCGTGGAGTGTGTGGACCTCTTGGTGGGGCCGGGGGCTGCCCAGGCAGCCATTAAGGAAGA CCCCTGGAACTGCTACATGTGCGGGCACAAGGGTACCTACGGGCTGCTGCGGCGGCGAGAGG ACTGGCCCTCCCGGCTCCAGATGTTCTTCGCTAATAACCACGACCAGGAATTTGACCCTCCA AAGGTTTACCCACCTGTCCCAGCTGAGAAGAGGAAGCCCATCCGGGTGCTGTCTCTCTTTGA TGGAATCGCTACAGGGCTCCTGGTGCTGAAGGACTTGGGCATTCAGGTGGACCGCTACATTG CCTCGGAGGTGTGTGAGGACTCCATCACGGTGGGCATGGTGCGGCACCAGGGGAAGATCATG TACGTCGGGGACGTCCGCAGCGTCACACAGAAGCATATCCAGGAGTGGGGCCCATTCGATCT GGTGATTGGGGGCAGTCCCTGCAATGACCTCTCCATCGTCAACCCTGCTCGCAAGGGCCTCT ACGAGGGCACTGGCCGGCTCTTCTTTGAGTTCTACCGCCTCCTGCATGATGCGCGGCCCAAG GAGGGAGATGATCGCCCCTTCTTCTGGCTCTTTGAGAATGTGGTGGCCATGGGCGTTAGTGA CAAGAGGGACATCTCGCGATTTCTCGAGTCCAACCCTGTGATGATTGATGCCAAAGAAGTGT CAGCTGCACACAGGGCCCGCTACTTCTGGGGTAACCTTCCCGGTATGAACAGGCCGTTGGCA TCCACTGTGAATGATAAGCTGGAGCTGCAGGAGTGTCTGGAGCATGGCAGGATAGCCAAGTT CAGCAAAGTGAGGACCATTACTACGAGGTCAAACTCCATAAAGCAGGGCAAAGACCAGCATT TTCCTGTCTTCATGAATGAGAAAGAGGACATCTTATGGTGCACTGAAATGGAAAGGGTATTT GGTTTCCCAGTCCACTATACTGACGTCTCCAACATGAGCCGCTTGGCGAGGCAGAGACTGCT GGGCCGGTCATGGAGCGTGCCAGTCATCCGCCACCTCTTCGCTCCGCTGAAGGAGTATTTTG CGTGTGTGTAA (SEQ ID NO: 593). An exemplary DHNMT3A gene sequence is provided at ENSEMBL Accession No. ENSG00000119772 (SEQ ID NO: 594).

By “Eukaryotic translation initiation factor 2A (EIF2A) polypeptide” is meant a protein having at least about 85% amino acid sequence identity to GenBank Accession No. AAH11885.1, which is provided below, or a fragment thereof having immunomodulatory activity.

>AAH11885.1 Eukaryotic translation initiation
factor 2A, 65 kDa [Homo sapiens]
(SEQ ID NO: 595)
MAPSTPLLTVRGSEGLYMVNGPPHFTESTVFPRESGKNCKVCIFS
KDGTLFAWGNGEKVNIISVTNKGLLHSFDLLKAVCLEFSPKNTVL
ATWQPYTTSKDGTAGIPNLQLYDVKTGTCLKSFIQKKMQNWCPSW
SEDETLCARNVNNEVHFFENNNFNTIANKLHLQKINDFVLSPGPQ
PYKVAVYVPGSKGAPSFVRLYQYPNFAGPHAALANKSFFKADKVT
MLWNKKATAVLVIASTDVDKTGASYYGEQTLHYIATNGESAVVQL
PKNGPIYDVVWNSSSTEFCAVYGFMPAKATIFNLKCDPVFDFGTG
PRNAAYYSPHGHILVLAGFGNLRGQMEVWDVKNYKLISKPVASDS
TYFAWCPDGEHILTATCAPRLRVNNGYKIWHYTGSILHKYDVPSN
AELWQVSWQPFLDGIFPAKTITYQAVPSEVPNEEPKVATAYRPPA
LRNKPITNSKLHEEEPPQNMKPQSGNDKPLSKTALKNQRKHEAKK
AAKQEARSDKSPDLAPTPAPQSTPRNTVSQSISGDPEIDKKIKNL
KKKLKAIEQLKEQAATGKQLEKNQLEKIQKETALLQELEDLKLGI.

By “Eukaryotic translation initiation factor 2A (EIF2A) polynucleotide” is meant a polynucleotide encoding a EIF2A polypeptide, as well as the introns, exons, 3′ untranslated regions, 5′ untranslated regions, and regulatory sequences associated with its expression, or fragments thereof. In embodiments, a EIF2A polynucleotide is the genomic sequence, cDNA, mRNA, or gene associated with and/or required for EIF2A expression. An exemplary EIF2A nucleic acid sequence is provided below (GenBank Accession No. BC011885.1:7-1764).

>BC011885.1:7-1764 Homo sapiens Eukaryotic Translation Initiation Factor 2A, 65 kDa, mRNA (cDNA Clone MGC:20557 IMAGE:3688407), Complete Cds
ATGGCGCCGTCCACGCCGCTCTTGACAGTCCGAGGATCAGAAGGACTGTACATGGTGAATGG ACCACCACATTTTACAGAAAGCACAGTGTTTCCAAGGGAATCTGGGAAGAATTGCAAAGTCT GTATCTTTAGTAAGGATGGGACCTTGTTTGCCTGGGGCAATGGAGAAAAAGTAAATATTATC AGTGTCACTAACAAGGGACTACTGCACTCCTTCGACCTCCTGAAGGCAGTTTGCCTTGAATT CTCACCCAAAAATACTGTCCTGGCAACGTGGCAGCCTTACACTACTTCTAAAGATGGCACAG CTGGGATACCCAACCTACAACTTTATGATGTGAAAACTGGGACATGTTTGAAATCTTTCATC CAGAAAAAAATGCAAAATTGGTGTCCATCCTGGTCAGAAGATGAAACTCTTTGTGCCCGCAA TGTTAACAATGAAGTTCACTTCTTTGAAAACAACAATTTTAACACAATTGCAAATAAATTGC ATTTGCAAAAAATTAATGACTTTGTATTATCACCTGGACCCCAACCATACAAGGTGGCTGTC TATGTTCCAGGAAGTAAAGGTGCACCTTCATTTGTTAGATTATATCAGTACCCCAACTTTGC TGGACCTCATGCAGCTTTAGCTAATAAAAGTTTCTTTAAGGCAGATAAAGTTACAATGCTGT GGAATAAAAAAGCTACTGCTGTGTTGGTAATAGCTAGCACAGATGTTGACAAGACAGGAGCT TCCTACTATGGAGAACAAACTCTACACTACATTGCAACAAATGGAGAAAGTGCTGTAGTGCA ATTACCAAAAAATGGCCCCATTTATGATGTAGTTTGGAATTCTAGTTCTACTGAGTTTTGTG CTGTATATGGTTTTATGCCTGCCAAAGCGACAATTTTCAACTTGAAATGTGATCCTGTATTT GACTTTGGAACTGGTCCTCGTAATGCAGCCTACTATAGCCCTCATGGACATATATTAGTATT AGCTGGATTTGGAAATCTGAGGGGACAAATGGAAGTGTGGGATGTGAAAAACTACAAACTTA TTTCTAAACCGGTGGCTTCTGATTCTACATATTTTGCTTGGTGCCCGGATGGTGAGCATATT TTAACAGCTACATGTGCTCCCAGGTTACGGGTTAATAATGGATACAAAATTTGGCATTATAC TGGCTCTATCTTGCACAAGTATGATGTGCCATCAAATGCAGAATTATGGCAGGTTTCTTGGC AGCCATTTTTGGATGGAATATTTCCAGCAAAAACAATAACTTACCAAGCAGTTCCAAGTGAA GTACCCAATGAGGAACCTAAAGTTGCAACAGCTTATAGACCCCCAGCTTTAAGAAATAAACC AATCACCAATTCCAAATTGCATGAAGAGGAACCACCTCAGAATATGAAACCACAATCAGGAA ACGATAAGCCATTATCAAAAACAGCTCTTAAAAATCAAAGGAAGCATGAAGCTAAGAAAGCT GCAAAGCAGGAAGCAAGAAGTGACAAGAGTCCAGATTTGGCACCTACTCCTGCCCCACAGAG CACACCACGAAACACTGTCTCTCAGTCAATTTCTGGGGACCCTGAGATAGACAAAAAAATCA AGAACCTAAAGAAGAAACTGAAAGCAATCGAACAACTGAAAGAACAAGCAGCAACTGGAAAA CAGCTAGAAAAAAATCAGTTGGAGAAATTCAGAAAGAAACAGCCCTTCTCCAGGAGCTGGA AGATTTGAAATTGGGTATTTAA (SEQ ID NO: 596). An exemplary EIF2A gene sequence is provided at ENSEMBL Accession No. ENSG00000144895 (SEQ ID NO: 597).

By “eomesodermin (EOMES) polypeptide” is meant a protein having at least about 85% amino acid sequence identity to GenBank accession number: CAB37939.1, provided below, or a fragment thereof and having immunomodulatory activity.

>CAB37939.1 eomesodermin protein, EOMES,
partial [Homo sapiens]
(SEQ ID NO: 598)
KQGRRMFPFLSFNINGLNPTAHYNVFVEVVLADPNHWRFQGGKWV
TSGKADNNMQGNKMYVHPESPNTGSHWMRQEISFGKLKLTNNKGA
NNNNTQMIVLQSLHKYQPRLHIVEVTEDGVEKDLNDPSKTQTFTF
SETQFIAVTAYQNTDITQLKIDHNPFAKGFR.

By “eomesodermin (EOMES) polynucleotide” is meant a polynucleotide encoding a EOMES polypeptide, as well as the introns, exons, 3′ untranslated regions, 5′ untranslated regions, and regulatory sequences associated with its expression, or fragments thereof. In embodiments, a EOMES polynucleotide is the genomic sequence, cDNA, mRNA, or gene associated with and/or required for EOMES expression. An exemplary EOMES nucleic acid sequence is provided below (GenBank Accession No. AJ010280.1).

>AJ010280.1 Homo sapiens mRNA Encoding Eomesodermin Protein, Partial
AAACAGGGCAGGCGCATGTTTCCTTTCTTGAGCTTCAACATAAACGGACTCAATCCCACTGC CCACTACAATGTGTTCGTAGAGGTGGTGCTGGCGGACCCCAACCACTGGCGCTTCCAGGGGG GCAAATGGGTGACCTCTGGCAAAGCCGACAATAACATGCAGGGCAACAAAATGTATGTTCAC CCAGAGTCTCCTAATACTGGTTCCCACTGGATGAGACAGGAGATTTCATTCGGGAAATTAAA ACTCACCAATAACAAAGGCGCAAATAACAACAACACCCAGATGATAGTCTTACAATCCTTAC ACAAATACCAACCCCGACTGCATATTGTTGAAGTTACAGAGGATGGCGTTGAGAAGGACTTG AATGATCCCTCAAAGACCCAGACTTTTACCTTCTCAGAAACGCAATTCATTGCTGTGACTGC CTACCAAAACACCGATATTACTCAACTAAAGATTGATCATAACCCCTTTGCAAAAGGCTTCA GA (SEQ ID NO: 599). An exemplary EOMES gene sequence is provided at ENSEMBL Accession No. ENSG00000163508 (SEQ ID NO: 600).

By “Friend leukemia integration 1 transcription factor (FLI-1 or FLI1) polypeptide” is meant a protein having at least about 85% amino acid sequence identity to GenBank Accession No. AAA58480.1, which is provided below, or a fragment thereof having immunomodulatory activity.

>AAA58480.1 FLI-1 [Homo sapiens]
(SEQ ID NO: 601)
MEGGLAGERARESPVDCSVSKCSKLVGGGESNPMNYNSYMDEKNG
PPPPNMTTNERRVIVPADATLVTQEHVRQWLEWAIKEYSLMEIDT
SFFQNMDGKELCKMNKEDFLRATTLYNTEVLLSHLSYLRESSLLA
YNTTSHTDQSSRLSVKEDPSYDSVRRGAWGNNMNSGLNKSPPLGG
AQTISKNTEQRPQPDPYQILGPTSSRLANPGSGQIQLWQFLLELL
SDSANASCITWEGINGEFKMTDPDEVARRWGQRKSKPNMNYDKLS
RALRYYYDKNIMTKVHGKRYAYKFDFHGIAQALQPHPTESSMYKY
PSDISYMPSYHAHQQKVNFVPPHPSSMPVTSSSFFGAASQYWTST
GGIYPNPNVPRHPNTHVPSHLGSYY.

By “Friend leukemia integration 1 transcription factor (FLI-1) polynucleotide” is meant a polynucleotide encoding a FLI-1 polypeptide, as well as the introns, exons, 3′ untranslated regions, 5′ untranslated regions, and regulatory sequences associated with its expression, or fragments thereof. In embodiments, a FLI-1 polynucleotide is the genomic sequence, cDNA, mRNA, or gene associated with and/or required for FLI-1 expression. An exemplary FLI-1 nucleic acid sequence is provided below (GenBank Accession No. M93255.1:543-1700).

>M93255.1:543-1700 Human FLI-1 mRNA, Complete Cds for Two Alternate Splicings
ATGGAGGGAGGACTGGCAGGCGAGCGGGCGAGGGAGTCTCCGGTGGACTGCAGCGTTAGCAA ATGCAGCAAGCTGGTGGGCGGAGGCGAGTCCAACCCCATGAACTACAACAGCTATATGGACG AGAAGAATGGCCCCCCTCCTCCCAACATGACCACCAACGAGAGGAGAGTCATCGTCCCCGCA GACGCCACACTGGTGACACAGGAGCATGTGAGGCAATGGCTGGAGTGGGCCATAAAGGAGTA CAGCTTGATGGAGATCGACACATCCTTTTTCCAGAACATGGATGGCAAGGAACTGTGTAAAA TGAACAAGGAGGACTTCCTCCGCGCCACCACCCTCTACAACACGGAAGTGCTGTTGTCACAC CTCAGTTACCTCAGGGAAAGTTCACTGCTGGCCTATAATACAACCTCCCACACCGACCAATC CTCACGATTGAGTGTCAAAGAAGACCCTTCTTATGACTCAGTCAGAAGAGGAGCTTGGGGCA ATAACATGAATTCTGGCCTCAACAAAAGTCCTCCCCTTGGAGGGGCACAAACGATCAGTAAG AATACAGAGCAACGGCCCCAGCCAGATCCGTATCAGATCCTGGGCCCGACCAGCAGTCGCCT AGCCAACCCTGGAAGCGGGCAGATCCAGCTGTGGCAATTCCTCCTGGAGCTGCTCTCCGACA GCGCCAACGCCAGCTGTATCACCTGGGAGGGGACCAACGGGGAGTTCAAAATGACGGACCCC GATGAGGTGGCCAGGCGCTGGGGGCAGCGGAAAAGCAAGCCCAACATGAATTACGACAAGCT GAGCCGGGCCCTCCGTTATTACTATGATAAAAACATTATGACCAAAGTGCACGGCAAAAGAT ATGCTTACAAATTTGACTTCCACGGCATTGCCCAGGCTCTGCAGCCACATCCGACCGAGTCG TCCATGTACAAGTACCCTTCTGACATCTCCTACATGCCTTCCTACCATGCCCACCAGCAGAA GGTGAACTTTGTCCCTCCCCATCCATCCTCCATGCCTGTCACTTCCTCCAGCTTCTTTGGAG CCGCATCACAATACTGGACCTCCACGGGGGGAATCTACCCCAACCCCAACGTCCCCCGCCAT CCTAACACCCACGTGCCTTCACACTTAGGCAGCTACTACTAG (SEQ ID NO: 602). An exemplary FLI-1 gene sequence is provided at ENSEMBL Accession No. ENSG00000151702 (SEQ ID NO: 603).

By “granzyme B (GZMB) polypeptide” is meant a protein having at least about 85% amino acid sequence identity to GenBank accession No. AAA75490.1, which is provided below, or a fragment thereof having immunomodulatory activity.

>AAA75490.1 granzyme B [Homo sapiens]
(SEQ ID NO: 604)
MQPILLLLAFLLLPRADAGEIIGGHEAKPHSRPYMAYLMIWDQKS
LKRCGGFLIQDDFVLTAAHCWGSSINVTLGAHNIKEQEPTQQFIP
VKRAIPHPAYNPKNFSNDIMLLQLERKAKRTRAVQPLRLPSNKAQ
VKPGQTCSVAGWGQTAPLGKHSHTLQEVKMTVQEDRKCESDLRHY
YDSTIELCVGDPEIKKTSFKGDSGGPLVCNKVAQGIVSYGRNNGM
PPRACTKVSSFVHWIKKTMKRY.

By “granzyme B (GZMB) polynucleotide” is meant a nucleic acid molecule encoding an GZMB polypeptide, as well as the introns, exons, 3′ untranslated regions, 5′ untranslated regions, and regulatory sequences associated with its expression, or fragments thereof. An exemplary GZMB polynucleotide sequence is provided below (GenBank Accession No. M28879.1:1161-1215,2256-2403,2857-2992,3200-3460,4105-4248).

### >M28879.1:1161-1215,2256-2403,2857-2992,3200-3460,4105-4248 Human Granzyme B (CTLA-1) Gene, Complete Cds

ATGCAACCAATCCTGCTTCTGCTGGCCTTCCTCCTGCTGCCCAGGGCAGATGCAGGGGAGAT CATCGGGGGACATGAGGCCAAGCCCCACTCCCGCCCCTACATGGCTTATCTTATGATCTGGG ATCAGAAGTCTCTGAAGAGGTGCGGTGGCTTCCTGATACAAGACGACTTCGTGCTGACAGCT GCTCACTGTTGGGGAAGCTCCATAAATGTCACCTTGGGGGCCCACAATATCAAGGAACAGGA GCCGACCCAGCAGTTTATCCCTGTGAAAAGAGCCATCCCCCATCCAGCCTATAATCCTAAGA ACTTCTCCAATGACATCATGCTACTGCAGCTGGAGAGAAAGGCCAAGCGGACCAGAGCTGTG CAGCCCCTCAGGCTACCTAGCAACAAGGCCCAGGTGAAGCCAGGGCAGACATGCAGTGTGGC CGGCTGGGGGCAGACGGCCCCCCTGGGAAAACACTCACACACACTACAAGAGGTGAAGATGA CAGTGCAGGAAGATCGAAAGTGCGAATCTGACTTACGCCATTATTACGACAGTACCATTGAG TTGTGCGTGGGGGACCCAGAGATTAAAAAGACTTCCTTTAAGGGGGACTCTGGAGGCCCTCT TGTGTGTAACAAGGTGGCCCAGGGCATTGTCTCCTATGGACGAAACAATGGCATGCCTCCAC GAGCCTGCACCAAAGTCTCAAGCTTTGTACACTGGATAAAGAAAACCATGAAACGCTACTAA (SEQ ID NO: 605). An exemplary human GZMB polynucleotide sequence is provided at Ensembl Accession No. ENSG00000100453 (SEQ ID NO: 606).

By “Inhibitor of DNA binding (ID3) polypeptide” is meant a protein having at least about 85% amino acid sequence identity to GenBank Accession No. CAA51827.1, which is provided below, or a fragment thereof having immunomodulatory activity.

>CAA51827.1 Id3 [Homo sapiens]
(SEQ ID NO: 607)
MKALSPVRGCYEAVCCLSERSLAIARGRGKGPAAEEPLSLLDDMN
HCYSRLRELVPGVPRGTQLSQVEILQRVIDYILDLQVVLAEPAPG
PPDGPHLPIQTAELAPELVISNDKRSFCH.

By “Inhibitor of DNA binding (ID3) polynucleotide” is meant a polynucleotide encoding a ID3 polypeptide, as well as the introns, exons, 3′ untranslated regions, 5′ untranslated regions, and regulatory sequences associated with its expression, or fragments thereof. In embodiments, a ID3 polynucleotide is the genomic sequence, cDNA, mRNA, or gene associated with and/or required for ID3 expression. An exemplary ID3 nucleic acid sequence is provided below (GenBank Accession No. X73428.1:739-1038,1146-1205).

>X73428.1:739-1038,1146-1205 H. sapiens Id3 Gene for HLH Type Transcription Factor
ATGAAGGCGCTGAGCCCGGTGCGCGGCTGCTACGAGGCGGTGTGCTGCCTGTCGGAACGCAG TCTGGCCATCGCCCGGGGCCGAGGGAAGGGCCCGGCAGCTGAGGAGCCGCTGAGCTTGCTGG ACGACATGAACCACTGCTACTCCCGCCTGCGGGAACTGGTACCCGGAGTCCCGAGAGGCACT CAGCTTAGCCAGGTGGAAATCCTACAGCGCGTCATCGACTACATTCTCGACCTGCAGGTAGT CCTGGCCGAGCCAGCCCCTGGACCCCCTGATGGCCCCCACCTTCCCATCCAGACAGCCGAGC TCGCTCCGGAACTTGTCATCTCCAACGACAAAAGGAGCTTTTGCCACTGA (SEQ ID NO: 608). An exemplary ID3 gene sequence is provided at ENSEMBL Accession No. ENSG00000117318 (SEQ ID NO: 609).

By “Ikaros family zinc finger 2 (IKZF2) polypeptide” is meant a protein having at least about 85% amino acid sequence identity to GenBank Accession No. AAH28936.1, which is provided below, or a fragment thereof having immunomodulatory activity.

>AAH28936.1 IKAROS family zinc finger 2
(Helios) [Homo sapiens]
(SEQ ID NO: 610)
METEAIDGYITCDNELSPEREHSNMAIDLTSSTPNGQHASPSHMT
STNSVKLEMQSDEECDRKPLSREDEIRGHDEGSSLEEPLIESSEV
ADNRKVQELQGEGGIRLPNGERPFHCNQCGASFTQKGNLLRHIKL
HSGEKPFKCPFCSYACRRRDALTGHLRTHSVGKPHKCNYCGRSYK
QRSSLEEHKERCHNYLQNVSMEAAGQVMSHHVPPMEDCKEQEPIM
DNNISLVPFERPAVIEKLTGNMGKRKSSTPQKFVGEKLMRFSYPD
IHFDMNLTYEKEAELMQSHMMDQAINNAITYLGAEALHPLMQHPP
STIAEVAPVISSAYSQVYHPNRIERPISRETADSHENNMDGPISL
IRPKSRPQEREASPSNSCLDSTDSESSHDDHQSYQGHPALNPKRK
QSPAYMKEDVKALDTTKAPKGSLKDIYKVFNGEGEQIRAFKCEHC
RVLFLDHVMYTIHMGCHGYRDPLECNICGYRSQDRYEFSSHIVRG
EHTFH.

By “Ikaros family zinc finger 2 (IKZF2) polynucleotide” is meant a polynucleotide encoding a IKZF2 polypeptide, as well as the introns, exons, 3′ untranslated regions, 5′ untranslated regions, and regulatory sequences associated with its expression, or fragments thereof. In embodiments, a IKZF2 polynucleotide is the genomic sequence, cDNA, mRNA, or gene associated with and/or required for IKZF2 expression. An exemplary IKZF2 nucleic acid sequence is provided below (GenBank Accession No. BC028936.1:317-1819).

>BC028936.1:317-1819 Homo sapiens IKAROS Family Zinc Finger 2 (Helios), mRNA (cDNA Clone MGC:34330 IMAGE:4540576), Complete Cds
ATGGAAACAGAGGCTATTGATGGCTATATAACGTGTGACAATGAGCTTTCACCCGAAAGGGA GCACTCCAATATGGCAATTGACCTCACCTCAAGCACACCCAATGGACAGCATGCCTCACCAA GTCACATGACAAGCACAAATTCAGTAAAGCTAGAAATGCAGAGTGATGAAGAGTGTGACAGG AAACCCCTGAGCCGTGAAGATGAGATCAGGGGCCATGATGAGGGTAGCAGCCTAGAAGAACC CCTAATTGAGAGCAGCGAGGTGGCTGACAACAGGAAAGTCCAGGAGCTTCAAGGCGAGGGAG GAATCCGGCTTCCGAATGGTGAACGCCCCTTCCACTGTAACCAGTGTGGAGCTTCTTTTACT CAGAAGGGCAACCTTCTGAGACACATAAAGTTACACTCTGGAGAGAAGCCGTTCAAATGTCC TTTCTGTAGCTACGCCTGTAGAAGAAGGGACGCCCTCACAGGACACCTCAGGACCCATTCTG TGGGTAAACCTCACAAGTGCAACTACTGTGGACGAAGCTACAAGCAGCGCAGTTCACTGGAG GAGCACAAGGAACGCTGCCACAACTATCTCCAGAATGTCAGCATGGAGGCTGCTGGGCAGGT CATGAGTCACCATGTACCTCCTATGGAAGATTGTAAGGAACAAGAGCCTATTATGGACAACA ATATTTCTCTGGTGCCTTTTGAGAGACCTGCTGTCATAGAGAAGCTCACGGGGAATATGGGA AAACGTAAAAGCTCCACTCCACAAAAGTTTGTGGGGGAAAAGCTCATGCGATTCAGCTACCC AGATATTCACTTTGATATGAACTTAACATATGAGAAGGAGGCTGAGCTGATGCAGTCTCATA TGATGGACCAAGCCATCAACAATGCAATCACCTACCTTGGAGCTGAGGCCCTTCACCCTCTG ATGCAGCACCCGCCAAGCACAATCGCTGAAGTGGCCCCAGTTATAAGCTCAGCTTATTCTCA GGTCTATCATCCAAATAGGATAGAAAGACCCATTAGCAGGGAAACTGCTGATAGTCATGAAA ACAACATGGATGGCCCCATCTCTCTCATCAGACCAAAGAGTCGACCCCAGGAAAGAGAGGCC TCTCCCAGCAATAGCTGCCTGGATTCCACTGACTCAGAAAGCAGCCATGATGACCACCAGTC CTACCAAGGACACCCTGCCTTAAATCCCAAGAGGAAACAAAGCCCAGCTTACATGAAGGAGG ATGTCAAAGCTTTGGATACTACCAAGGCTCCTAAGGGCTCTCTGAAGGACATCTACAAGGTC TTCAATGGAGAAGGAGAACAGATTAGGGCCTTCAAGTGTGAGCACTGCCGAGTCCTTTTCCT AGACCATGTCATGTACACCATTCACATGGGTTGCCATGGCTACCGGGACCCACTGGAATGCA ACATCTGTGGCTACAGAAGCCAGGACCGTTATGAGTTTTCATCACACATTGTTCGAGGGGAG CACACATTCCACTAG (SEQ ID NO: 611). An exemplary IKZF2 gene sequence is provided at ENSEMBL Accession No. ENSG00000030419 (SEQ ID NO: 612).

By “Interleukin 6 (IL-6) polypeptide” is meant a protein having at least about 85% amino acid sequence identity to GenBank Accession No. AAC41704.1, which is provided below, or a fragment thereof having immunomodulatory activity.

>AAC41704.1 interleukin 6 [Homo sapiens]
(SEQ ID NO: 613)
MNSFSTSAFGPVAFSLGLLLVLPAAFPAPVPPGEDSKDVAAPHRQ
PLTSSERIDKQIRYILDGISALRKETCNKSNMCESSKEALAENNL
NLPKMAEKDGCFQSGFNEETCLVKIITGLLEFEVYLEYLQNRFES
SEEQARAVQMSTKVLIQFLQKKAKNLDAITTPDPTTNASLLTKLQ
AQNQWLQDMTTHLILRSFKEFLQSSLRALRQM.

By “Interleukin 6 (IL-6) polynucleotide” is meant a polynucleotide encoding a IL-6 polypeptide, as well as the introns, exons, 3′ untranslated regions, 5′ untranslated regions, and regulatory sequences associated with its expression, or fragments thereof. In embodiments, a IL-6 polynucleotide is the genomic sequence, cDNA, mRNA, or gene associated with and/or required for IL-6 expression. An exemplary IL-6 nucleic acid sequence is provided below (GenBank Accession No. M54894.1:51-689).

>M54894.1:51-689 Human Interleukin 6 mRNA, Complete Cds
ATGAACTCCTTCTCCACAAGCGCCTTCGGTCCAGTTGCCTTCTCCCTGGGGCTGCTCCTGGT GTTGCCTGCTGCCTTCCCTGCCCCAGTACCCCCAGGAGAAGATTCCAAAGATGTAGCCGCCC CACACAGACAGCCACTCACCTCTTCAGAACGAATTGACAAACAAATTCGGTACATCCTCGAC GGCATCTCAGCCCTGAGAAAGGAGACATGTAACAAGAGTAACATGTGTGAAAGCAGCAAAGA GGCACTGGCAGAAAACAACCTGAACCTTCCAAAGATGGCTGAAAAAGATGGATGCTTCCAAT CTGGATTCAATGAGGAGACTTGCCTGGTGAAAATCATCACTGGTCTTTTGGAGTTTGAGGTA TACCTAGAGTACCTCCAGAACAGATTTGAGAGTAGTGAGGAACAAGCCAGAGCTGTGCAGAT GAGTACAAAAGTCCTGATCCAGTTCCTGCAGAAAAAGGCAAAGAATCTAGATGCAATAACCA CCCCTGACCCAACCACAAATGCCAGCCTGCTGACGAAGCTGCAGGCACAGAACCAGTGGCTG CAGGACATGACAACTCATCTCATTCTGCGCAGCTTTAAGGAGTTCCTGCAGTCCAGCCTGAG GGCTCTTCGGCAAATGTAG (SEQ ID NO: 614). An exemplary IL-6 gene sequence is provided at ENSEMBL Accession No. ENSG00000136244 (SEQ ID NO: 615).

By “interferon gamma (IFN-G) polypeptide” is meant a protein having at least about 85% amino acid sequence identity to GenBank accession No. CAA44325.1, which is provided below, or a functional fragment thereof having immunomodulatory activity.

>CAA44325.1 interferon-gamma [Homo sapiens]
(SEQ ID NO: 616)
MQDPYVKEAENLKKYFNAGHSDVADNGTLFLGILKNWKEESDRKI
MQSQIVSFYFKLFKNFKDDQSIQKSVETIKEDMNVKFFNSNKKKR
DDFEKLTNYSVTDLNVQRKAIHELIQVMAELSPAAKTGKRKRSQM
LFRGRRASQ.

By “interferon gamma (IFN-G; INFg) polynucleotide” is meant a nucleic acid molecule encoding an IFN-G polypeptide, as well as the introns, exons, 3′ untranslated regions, 5′ untranslated regions, and regulatory sequences associated with its expression, or fragments thereof. An exemplary INFg polynucleotide sequence is provided below (GenBank Accession No. X62468.1:13-447).

>X62468.1:13-447 H. sapiens mRNA for IFN-Gamma (pKC-0)
ATGCAAGACCCATATGTAAAAGAAGCAGAAAACCTTAAGAAATATTTTAATGCAGGTCATTC AGATGTAGCGGATAATGGAACTCTTTTCTTAGGCATTTTGAAGAATTGGAAAGAGGAGAGTG ACAGAAAAATAATGCAGAGCCAAATTGTCTCCTTTTACTTCAAACTTTTTAAAAACTTTAAA GATGACCAGAGCATCCAAAAGAGTGTGGAGACCATCAAGGAAGACATGAATGTCAAGTTTTT CAATAGCAACAAAAAGAAACGAGATGACTTCGAAAAGCTGACTAATTATTCGGTAACTGACT TGAATGTCCAACGCAAAGCAATACATGAACTCATCCAAGTGATGGCTGAACTGTCGCCAGCA GCTAAAACAGGGAAGCGAAAAAGGAGTCAGATGCTGTTTCGAGGTCGAAGAGCATCCCAGTA A (SEQ ID NO: 617). An exemplary human IFN-G polynucleotide sequence is provided at Ensembl Accession No. ENSG00000111537 (SEQ ID NO: 618).

By “interleukin-2 (IL-2) polypeptide” is meant a protein having at least about 85% amino acid sequence identity to GenBank Accession No. CAA23827.1, provided below, or a fragment thereof and having immunomodulatory activity.

>CAA23827.1 interleukin-2 [Homo sapiens]
(SEQ ID NO: 619)
MYRMQLLSCIALSLALVTNSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT.

By “interleukin-2 (IL-2) polynucleotide” is meant a polynucleotide encoding an IL-2 polypeptide, as well as the introns, exons, 3′ untranslated regions, 5′ untranslated regions, and regulatory sequences associated with its expression, or fragments thereof. In embodiments, an IL-2 polynucleotide is the genomic sequence, cDNA, mRNA, or gene associated with and/or required for IL-2 expression. An exemplary IL-2 nucleic acid sequence is provided below (GenBank Accession No. V00564.1:48-509).

>V00564.1:48-509 Human mRNA Encoding Interleukin-2 (IL-2) a Lymphocyte Regulatory Molecule
ATGTACAGGATGCAACTCCTGTCTTGCATTGCACTAAGTCTTGCACTTGTCACAAACAGTGC ACCTACTTCAAGTTCTACAAAGAAAACACAGCTACAACTGGAGCATTTACTGCTGGATTTAC AGATGATTTTGAATGGAATTAATAATTACAAGAATCCCAAACTCACCAGGATGCTCACATTT AAGTTTTACATGCCCAAGAAGGCCACAGAACTGAAACATCTTCAGTGTCTAGAAGAAGAACT CAAACCTCTGGAGGAAGTGCTAAATTTAGCTCAAAGCAAAAACTTTCACTTAAGACCCAGGG ACTTAATCAGCAATATCAACGTAATAGTTCTGGAACTAAAGGGATCTGAAACAACATTCATG TGTGAATATGCTGATGAGACAGCAACCATTGTAGAATTTCTGAACAGATGGATTACCTTTTG TCAAAGCATCATCTCAACACTAACTTGA (SEQ ID NO: 620). An exemplary Interleukin-2 gene sequence is provided at ENSEMBL Accession No. ENSG00000057657 (SEQ ID NO: 621).

By “Marker of Proliferation KI67(KI67) polypeptide” is meant a protein having at least about 85% amino acid sequence identity to NCBI Reference Sequence No. NP 002408.3, provided below, or a fragment thereof and having immunomodulatory activity.

>NP_002408.3 proliferation marker protein
Ki-67 isoform 1
[Homo sapiens]
(SEQ ID NO: 622)
MWPTRRLVTIKRSGVDGPHFPLSLSTCLFGRGIECDIRIQLPVVS
KQHCKIETHEQEAILHNFSSTNPTQVNGSVIDEPVRLKHGDVITI
IDRSFRYENESLQNGRKSTEFPRKIREQEPARRVSRSSFSSDPDE
KAQDSKAYSKITEGKVSGNPQVHIKNVKEDSTADDSKDSVAQGTT
NVHSSEHAGRNGRNAADPISGDFKEISSVKLVSRYGELKSVPTTQ
CLDNSKKNESPFWKLYESVKKELDVKSQKENVLQYCRKSGLQTDY
ATEKESADGLQGETQLLVSRKSRPKSGGSGHAVAEPASPEQELDQ
NKGKGRDVESVQTPSKAVGASFPLYEPAKMKTPVQYSQQQNSPQK
HKNKDLYTTGRRESVNLGKSEGFKAGDKTLTPRKLSTRNRTPAKV
EDAADSATKPENLSSKTRGSIPTDVEVLPTETEIHNEPFLTLWLT
QVERKIQKDSLSKPEKLGTTAGQMCSGLPGLSSVDINNFGDSINE
SEGIPLKRRRVSFGGHLRPELFDENLPPNTPLKRGEAPTKRKSLV
MHTPPVLKKIIKEQPQPSGKQESGSEIHVEVKAQSLVISPPAPSP
RKTPVASDQRRRSCKTAPASSSKSQTEVPKRGGRKSGNLPSKRVS
ISRSQHDILQMICSKRRSGASEANLIVAKSWADVVKLGAKQTQTK
VIKHGPQRSMNKRQRRPATPKKPVGEVHSQFSTGHANSPCTIIIG
KAHTEKVHVPARPYRVLNNFISNQKMDFKEDLSGIAEMFKTPVKE
QPQLTSTCHIAISNSENLLGKQFQGTDSGEEPLLPTSESFGGNVF
FSAQNAAKQPSDKCSASPPLRRQCIRENGNVAKTPRNTYKMTSLE
TKTSDTETEPSKTVSTANRSGRSTEFRNIQKLPVESKSEETNTEI
VECILKRGQKATLLQQRREGEMKEIERPFETYKENIELKENDEKM
KAMKRSRTWGQKCAPMSDLTDLKSLPDTELMKDTARGQNLLQTQD
HAKAPKSEKGKITKMPCQSLQPEPINTPTHTKQQLKASLGKVGVK
EELLAVGKFTRTSGETTHTHREPAGDGKSIRTFKESPKQILDPAA
RVTGMKKWPRTPKEEAQSLEDLAGFKELFQTPGPSEESMTDEKTT
KIACKSPPPESVDTPTSTKQWPKRSLRKADVEEEFLALRKLTPSA
GKAMLTPKPAGGDEKDIKAFMGTPVQKLDLAGTLPGSKRQLQTPK
EKAQALEDLAGFKELFQTPGHTEELVAAGKTTKIPCDSPQSDPVD
TPTSTKQRPKRSIRKADVEGELLACRNLMPSAGKAMHTPKPSVGE
EKDIIIFVGTPVQKLDLTENLTGSKRRPQTPKEEAQALEDLTGFK
ELFQTPGHTEEAVAAGKTTKMPCESSPPESADTPTSTRRQPKTPL
EKRDVQKELSALKKLTQTSGETTHTDKVPGGEDKSINAFRETAKQ
KLDPAASVTGSKRHPKTKEKAQPLEDLAGLKELFQTPVCTDKPTT
HEKTTKIACRSQPDPVDTPTSSKPQSKRSLRKVDVEEEFFALRKR
TPSAGKAMHTPKPAVSGEKNIYAFMGTPVQKLDLTENLTGSKRRL
QTPKEKAQALEDLAGFKELFQTRGHTEESMTNDKTAKVACKSSQP
DPDKNPASSKRRLKTSLGKVGVKEELLAVGKLTQTSGETTHTHTE
PTGDGKSMKAFMESPKQILDSAASLTGSKRQLRTPKGKSEVPEDL
AGFIELFQTPSHTKESMTNEKTTKVSYRASQPDLVDTPTSSKPQP
KRSLRKADTEEEFLAFRKQTPSAGKAMHTPKPAVGEEKDINTFLG
TPVQKLDQPGNLPGSNRRLQTRKEKAQALEELTGFRELFQTPCTD
NPTTDEKTTKKILCKSPQSDPADTPTNTKQRPKRSLKKADVEEEF
LAFRKLTPSAGKAMHTPKAAVGEEKDINTFVGTPVEKLDLLGNLP
GSKRRPQTPKEKAKALEDLAGFKELFQTPGHTEESMTDDKITEVS
CKSPQPDPVKTPTSSKQRLKISLGKVGVKEEVLPVGKLTQTSGKT
TQTHRETAGDGKSIKAFKESAKQMLDPANYGTGMERWPRTPKEEA
QSLEDLAGFKELFQTPDHTEESTTDDKTTKIACKSPPPESMDTPT
STRRRPKTPLGKRDIVEELSALKQLTQTTHTDKVPGDEDKGINVF
RETAKQKLDPAASVTGSKRQPRTPKGKAQPLEDLAGLKELFQTPI
CTDKPTTHEKTTKIACRSPQPDPVGTPTIFKPQSKRSLRKADVEE
ESLALRKRTPSVGKAMDTPKPAGGDEKDMKAFMGTPVQKLDLPGN
LPGSKRWPQTPKEKAQALEDLAGFKELFQTPGTDKPTTDEKTTKI
ACKSPQPDPVDTPASTKQRPKRNLRKADVEEEFLALRKRTPSAGK
AMDTPKPAVSDEKNINTFVETPVQKLDLLGNLPGSKRQPQTPKEK
AEALEDLVGFKELFQTPGHTEESMTDDKITEVSCKSPQPESFKTS
RSSKQRLKIPLVKVDMKEEPLAVSKLTRTSGETTQTHTEPTGDSK
SIKAFKESPKQILDPAASVTGSRRQLRTRKEKARALEDLVDFKEL
FSAPGHTEESMTIDKNTKIPCKSPPPELTDTATSTKRCPKTRPRK
EVKEELSAVERLTQTSGQSTHTHKEPASGDEGIKVLKQRAKKKPN
PVEEEPSRRRPRAPKEKAQPLEDLAGFTELSETSGHTQESLTAGK
ATKIPCESPPLEVVDTTASTKRHLRTRVQKVQVKEEPSAVKFTQT
SGETTDADKEPAGEDKGIKALKESAKQTPAPAASVTGSRRRPRAP
RESAQAIEDLAGFKDPAAGHTEESMTDDKTTKIPCKSSPELEDTA
TSSKRRPRTRAQKVEVKEELLAVGKLTQTSGETTHTDKEPVGEGK
GTKAFKQPAKRKLDAEDVIGSRRQPRAPKEKAQPLEDLASFQELS
QTPGHTEELANGAADSFTSAPKQTPDSGKPLKISRRVLRAPKVEP
VGDVVSTRDPVKSQSKSNTSLPPLPFKRGGGKDGSVTGTKRLRCM
PAPEEIVEELPASKKQRVAPRARGKSSEPVVIMKRSLRTSAKRIE
PAEELNSNDMKTNKEEHKLQDSVPENKGISLRSRRQNKTEAEQQI
TEVFVLAERIEINRNEKKPMKTSPEMDIQNPDDGARKPIPRDKVT
ENKRCLRSARQNESSQPKVAEESGGQKSAKVLMQNQKGKGEAGNS
DSMCLRSRKTKSQPAASTLESKSVQRVTRSVKRCAENPKKAEDNV
CVKKIRTRSHRDSEDI.

By “Homo sapiens marker of proliferation Ki-67(Ki67) polynucleotide” is meant a polynucleotide encoding a Ki67 polypeptide, as well as the introns, exons, 3′ untranslated regions, 5′ untranslated regions, and regulatory sequences associated with its expression, or fragments thereof. In embodiments, a Ki67 polynucleotide is the genomic sequence, cDNA, mRNA, or gene associated with and/or required for Ki67 expression. An exemplary Ki67 nucleic acid sequence is provided below (NCBI Ref. Seq. No. NM_002417.5:415-10185).

>NM_002417.5:415-10185 Homo sapiens Marker of Proliferation Ki-67 (MKI67), Transcript Variant 1, mRNA
ATGTGGCCCACGAGACGCCTGGTTACTATCAAAAGGAGCGGGGTCGACGGTCCCCACTTTCC CCTGAGCCTCAGCACCTGCTTGTTTGGAAGGGGTATTGAATGTGACATCCGTATCCAGCTTC CTGTTGTGTCAAAACAACATTGCAAAATTGAAATCCATGAGCAGGAGGCAATATTACATAAT TTCAGTTCCACAAATCCAACACAAGTAAATGGGTCTGTTATTGATGAGCCTGTACGGCTAAA ACATGGAGATGTAATAACTATTATTGATCGTTCCTTCAGGTATGAAAATGAAAGTCTTCAGA ATGGAAGGAAGTCAACTGAATTTCCAAGAAAAATACGTGAACAGGAGCCAGCACGTCGTGTC TCAAGATCTAGCTTCTCTTCTGACCCTGATGAGAAAGCTCAAGATTCCAAGGCCTATTCAAA AATCACTGAAGGAAAAGTTTCAGGAAATCCTCAGGTACATATCAAGAATGTCAAAGAAGACA GTACCGCAGATGACTCAAAAGACAGTGTTGCTCAGGGAACAACTAATGTTCATTCCTCAGAA CATGCTGGACGTAATGGCAGAAATGCAGCTGATCCCATTTCTGGGGATTTTAAAGAAATTTC CAGCGTTAAATTAGTGAGCCGTTATGGAGAATTGAAGTCTGTTCCCACTACACAATGTCTTG ACAATAGCAAAAAAAATGAATCTCCCTTTTGGAAGCTTTATGAGTCAGTGAAGAAAGAGTTG GATGTAAAATCACAAAAAGAAAATGTCCTACAGTATTGTAGAAAATCTGGATTACAAACTGA TTACGCAACAGAGAAAGAAAGTGCTGATGGTTTACAGGGGGAGACCCAACTGTTGGTCTCGC GTAAGTCAAGACCAAAATCTGGTGGGAGCGGCCACGCTGTGGCAGAGCCTGCTTCACCTGAA CAAGAGCTTGACCAGAACAAGGGGAAGGGAAGAGACGTGGAGTCTGTTCAGACTCCCAGCAA GGCTGTGGGCGCCAGCTTTCCTCTCTATGAGCCGGCTAAAATGAAGACCCCTGTACAATATT CACAGCAACAAAATTCTCCACAAAAACATAAGAACAAAGACCTGTATACTACTGGTAGAAGA GAATCTGTGAATCTGGGTAAAAGTGAAGGCTTCAAGGCTGGTGATAAAACTCTTACTCCCAG GAAGCTTTCAACTAGAAATCGAACACCAGCTAAAGTTGAAGATGCAGCTGACTCTGCCACTA AGCCAGAAAATCTCTCTTCCAAAACCAGAGGAAGTATTCCTACAGATGTGGAAGTTCTGCCT ACGGAAACTGAAATTCACAATGAGCCATTTTTAACTCTGTGGCTCACTCAAGTTGAGAGGAA GATCCAAAAGGATTCCCTCAGCAAGCCTGAGAAATTGGGCACTACAGCTGGACAGATGTGCT CTGGGTTACCTGGTCTTAGTTCAGTTGATATCAACAACTTTGGTGATTCCATTAATGAGAGT GAGGGAATACCTTTGAAAAGAAGGCGTGTGTCCTTTGGTGGGCACCTAAGACCTGAACTATT TGATGAAAACTTGCCTCCTAATACGCCTCTCAAAAGGGGAGAAGCCCCAACCAAAAGAAAGT CTCTGGTAATGCACACTCCACCTGTCCTGAAGAAAATCATCAAGGAACAGCCTCAACCATCA GGAAAACAAGAGTCAGGTTCAGAAATCCATGTGGAAGTGAAGGCACAAAGCTTGGTTATAAG CCCTCCAGCTCCTAGTCCTAGGAAAACTCCAGTTGCCAGTGATCAACGCCGTAGGTCCTGCA AAACAGCCCCTGCTTCCAGCAGCAAATCTCAGACAGAGGTTCCTAAGAGAGGAGGGAGAAAG AGTGGCAACCTGCCTTCAAAGAGAGTGTCTATCAGCCGAAGTCAACATGATATTTTACAGAT GATATGTTCCAAAAGAAGAAGTGGTGCTTCGGAAGCAAATCTGATTGTTGCAAAATCATGGG CAGATGTAGTAAAACTTGGTGCAAAACAAACACAAACTAAAGTCATAAAACATGGTCCTCAA AGGTCAATGAACAAAAGGCAAAGAAGACCTGCTACTCCAAAGAAGCCTGTGGGCGAAGTTCA CAGTCAATTTAGTACAGGCCACGCAAACTCTCCTTGTACCATAATAATAGGGAAAGCTCATA CTGAAAAAGTACATGTGCCTGCTCGACCCTACAGAGTGCTCAACAACTTCATTTCCAACCAA AAAATGGACTTTAAGGAAGATCTTTCAGGAATAGCTGAAATGTTCAAGACCCCAGTGAAGGA GCAACCGCAGTTGACAAGCACATGTCACATCGCTATTTCAAATTCAGAGAATTTGCTTGGAA AACAGTTTCAAGGAACTGATTCAGGAGAAGAACCTCTGCTCCCCACCTCAGAGAGTTTTGGA GGAAATGTGTTCTTCAGTGCACAGAATGCAGCAAAACAGCCATCTGATAAATGCTCTGCAAG CCCTCCCTTAAGACGGCAGTGTATTAGAGAAAATGGAAACGTAGCAAAAACGCCCAGGAACA CCTACAAAATGACTTCTCTGGAGACAAAAACTTCAGATACTGAGACAGAGCCTTCAAAAACA GTATCCACTGCAAACAGGTCAGGAAGGTCTACAGAGTTCAGGAATATACAGAAGCTACCTGT GGAAAGTAAGAGTGAAGAAACAAATACAGAAATTGTTGAGTGCATCCTAAAAAGAGGTCAGA AGGCAACACTACTACAACAAAGGAGAGAAGGAGAGATGAAGGAAATAGAAAGACCTTTTGAG ACATATAAGGAAAATATTGAATTAAAAGAAAACGATGAAAAGATGAAAGCAATGAAGAGATC AAGAACTTGGGGGCAGAAATGTGCACCAATGTCTGACCTGACAGACCTCAAGAGCTTGCCTG ATACAGAACTCATGAAAGACACGGCACGTGGCCAGAATCTCCTCCAAACCCAAGATCATGCC AAGGCACCAAAGAGTGAGAAAGGCAAAATCACTAAAATGCCCTGCCAGTCATTACAACCAGA ACCAATAAACACCCCAACACACACAAAACAACAGTTGAAGGCATCCCTGGGGAAAGTAGGTG TGAAAGAAGAGCTCCTAGCAGTCGGCAAGTTCACACGGACGTCAGGGGAGACCACGCACACG CACAGAGAGCCAGCAGGAGATGGCAAGAGCATCAGAACGTTTAAGGAGTCTCCAAAGCAGAT CCTGGACCCAGCAGCCCGTGTAACTGGAATGAAGAAGTGGCCAAGAACGCCTAAGGAAGAGG CCCAGTCACTAGAAGACCTGGCTGGCTTCAAAGAGCTCTTCCAGACACCAGGTCCCTCTGAG GAATCAATGACTGATGAGAAAACTACCAAAATAGCCTGCAAATCTCCACCACCAGAATCAGT GGACACTCCAACAAGCACAAAGCAATGGCCTAAGAGAAGTCTCAGGAAAGCAGATGTAGAGG AAGAATTCTTAGCACTCAGGAAACTAACACCATCAGCAGGGAAAGCCATGCTTACGCCCAAA CCAGCAGGAGGTGATGAGAAAGACATTAAAGCATTTATGGGAACTCCAGTGCAGAAACTGGA CCTGGCAGGAACTTTACCTGGCAGCAAAAGACAGCTACAGACTCCTAAGGAAAAGGCCCAGG CTCTAGAAGACCTGGCTGGCTTTAAAGAGCTCTTCCAGACTCCTGGTCACACCGAGGAATTA GTGGCTGCTGGTAAAACCACTAAAATACCCTGCGACTCTCCACAGTCAGACCCAGTGGACAC CCCAACAAGCACAAAGCAACGACCCAAGAGAAGTATCAGGAAAGCAGATGTAGAGGGAGAAC TCTTAGCGTGCAGGAATCTAATGCCATCAGCAGGCAAAGCCATGCACACGCCTAAACCATCA GTAGGTGAAGAGAAAGACATCATCATATTTGTGGGAACTCCAGTGCAGAAACTGGACCTGAC AGAGAACTTAACCGGCAGCAAGAGACGGCCACAAACTCCTAAGGAAGAGGCCCAGGCTCTGG AAGACCTGACTGGCTTTAAAGAGCTCTTCCAGACCCCTGGTCATACTGAAGAAGCAGTGGCT GCTGGCAAAACTACTAAAATGCCCTGCGAATCTTCTCCACCAGAATCAGCAGACACCCCAAC AAGCACAAGAAGGCAGCCCAAGACACCTTTGGAGAAAAGGGACGTACAGAAGGAGCTCTCAG CCCTGAAGAAGCTCACACAGACATCAGGGGAAACCACACACACAGATAAAGTACCAGGAGGT GAGGATAAAAGCATCAACGCGTTTAGGGAAACTGCAAAACAGAAACTGGACCCAGCAGCAAG TGTAACTGGTAGCAAGAGGCACCCAAAAACTAAGGAAAAGGCCCAACCCCTAGAAGACCTGG CTGGCTTGAAAGAGCTCTTCCAGACACCAGTATGCACTGACAAGCCCACGACTCACGAGAAA ACTACCAAAATAGCCTGCAGATCACAACCAGACCCAGTGGACACACCAACAAGCTCCAAGCC ACAGTCCAAGAGAAGTCTCAGGAAAGTGGACGTAGAAGAAGAATTCTTCGCACTCAGGAAAC GAACACCATCAGCAGGCAAAGCCATGCACACACCCAAACCAGCAGTAAGTGGTGAGAAAAAC ATCTACGCATTTATGGGAACTCCAGTGCAGAAACTGGACCTGACAGAGAACTTAACTGGCAG CAAGAGACGGCTACAAACTCCTAAGGAAAAGGCCCAGGCTCTAGAAGACCTGGCTGGCTTTA AAGAGCTCTTCCAGACACGAGGTCACACTGAGGAATCAATGACTAACGATAAAACTGCCAAA GTAGCCTGCAAATCTTCACAACCAGACCCAGACAAAAACCCAGCAAGCTCCAAGCGACGGCT CAAGACATCCCTGGGGAAAGTGGGCGTGAAAGAAGAGCTCCTAGCAGTTGGCAAGCTCACAC AGACATCAGGAGAGACTACACACACACACACAGAGCCAACAGGAGATGGTAAGAGCATGAAA GCATTTATGGAGTCTCCAAAGCAGATCTTAGACTCAGCAGCAAGTCTAACTGGCAGCAAGAG GCAGCTGAGAACTCCTAAGGGAAAGTCTGAAGTCCCTGAAGACCTGGCCGGCTTCATCGAGC TCTTCCAGACACCAAGTCACACTAAGGAATCAATGACTAACGAAAAAACTACCAAAGTATCC TACAGAGCTTCACAGCCAGACCTAGTGGACACCCCAACAAGCTCCAAGCCACAGCCCAAGAG AAGTCTCAGGAAAGCAGACACTGAAGAAGAATTTTTAGCATTTAGGAAACAAACGCCATCAG CAGGCAAAGCCATGCACACACCCAAACCAGCAGTAGGTGAAGAGAAAGACATCAACACGTTT TTGGGAACTCCAGTGCAGAAACTGGACCAGCCAGGAAATTTACCTGGCAGCAATAGACGGCT ACAAACTCGTAAGGAAAAGGCCCAGGCTCTAGAAGAACTGACTGGCTTCAGAGAGCTTTTCC AGACACCATGCACTGATAACCCCACGACTGATGAGAAAACTACCAAAAAAATACTCTGCAAA TCTCCGCAATCAGACCCAGCGGACACCCCAACAAACACAAAGCAACGGCCCAAGAGAAGCCT CAAGAAAGCAGACGTAGAGGAAGAATTTTTAGCATTCAGGAAACTAACACCATCAGCAGGCA AAGCCATGCACACGCCTAAAGCAGCAGTAGGTGAAGAGAAAGACATCAACACATTTGTGGGG ACTCCAGTGGAGAAACTGGACCTGCTAGGAAATTTACCTGGCAGCAAGAGACGGCCACAAAC TCCTAAAGAAAAGGCCAAGGCTCTAGAAGATCTGGCTGGCTTCAAAGAGCTCTTCCAGACAC CAGGTCACACTGAGGAATCAATGACCGATGACAAAATCACAGAAGTATCCTGCAAATCTCCA CAACCAGACCCAGTCAAAACCCCAACAAGCTCCAAGCAACGACTCAAGATATCCTTGGGGAA AGTAGGTGTGAAAGAAGAGGTCCTACCAGTCGGCAAGCTCACACAGACGTCAGGGAAGACCA CACAGACACACAGAGAGACAGCAGGAGATGGAAAGAGCATCAAAGCGTTTAAGGAATCTGCA AAGCAGATGCTGGACCCAGCAAACTATGGAACTGGGATGGAGAGGTGGCCAAGAACACCTAA GGAAGAGGCCCAATCACTAGAAGACCTGGCCGGCTTCAAAGAGCTCTTCCAGACACCAGACC ACACTGAGGAATCAACAACTGATGACAAAACTACCAAAATAGCCTGCAAATCTCCACCACCA GAATCAATGGACACTCCAACAAGCACAAGGAGGCGGCCCAAAACACCTTTGGGGAAAAGGGA TATAGTGGAAGAGCTCTCAGCCCTGAAGCAGCTCACACAGACCACACACACAGACAAAGTAC CAGGAGATGAGGATAAAGGCATCAACGTGTTCAGGGAAACTGCAAAACAGAAACTGGACCCA GCAGCAAGTGTAACTGGTAGCAAGAGGCAGCCAAGAACTCCTAAGGGAAAAGCCCAACCCCT AGAAGACTTGGCTGGCTTGAAAGAGCTCTTCCAGACACCAATATGCACTGACAAGCCCACGA CTCATGAGAAAACTACCAAAATAGCCTGCAGATCTCCACAACCAGACCCAGTGGGTACCCCA ACAATCTTCAAGCCACAGTCCAAGAGAAGTCTCAGGAAAGCAGACGTAGAGGAAGAATCCTT AGCACTCAGGAAACGAACACCATCAGTAGGGAAAGCTATGGACACACCCAAACCAGCAGGAG GTGATGAGAAAGACATGAAAGCATTTATGGGAACTCCAGTGCAGAAATTGGACCTGCCAGGA AATTTACCTGGCAGCAAAAGATGGCCACAAACTCCTAAGGAAAAGGCCCAGGCTCTAGAAGA CCTGGCTGGCTTCAAAGAGCTCTTCCAGACACCAGGCACTGACAAGCCCACGACTGATGAGA AAACTACCAAAATAGCCTGCAAATCTCCACAACCAGACCCAGTGGACACCCCAGCAAGCACA AAGCAACGGCCCAAGAGAAACCTCAGGAAAGCAGACGTAGAGGAAGAATTTTTAGCACTCAG GAAACGAACACCATCAGCAGGCAAAGCCATGGACACACCAAAACCAGCAGTAAGTGATGAGA AAAATATCAACACATTTGTGGAAACTCCAGTGCAGAAACTGGACCTGCTAGGAAATTTACCT GGCAGCAAGAGACAGCCACAGACTCCTAAGGAAAAGGCTGAGGCTCTAGAGGACCTGGTTGG CTTCAAAGAACTCTTCCAGACACCAGGTCACACTGAGGAATCAATGACTGATGACAAAATCA CAGAAGTATCCTGTAAATCTCCACAGCCAGAGTCATTCAAAACCTCAAGAAGCTCCAAGCAA AGGCTCAAGATACCCCTGGTGAAAGTGGACATGAAAGAAGAGCCCCTAGCAGTCAGCAAGCT CACACGGACATCAGGGGAGACTACGCAAACACACACAGAGCCAACAGGAGATAGTAAGAGCA TCAAAGCGTTTAAGGAGTCTCCAAAGCAGATCCTGGACCCAGCAGCAAGTGTAACTGGTAGC AGGAGGCAGCTGAGAACTCGTAAGGAAAAGGCCCGTGCTCTAGAAGACCTGGTTGACTTCAA AGAGCTCTTCTCAGCACCAGGTCACACTGAAGAGTCAATGACTATTGACAAAAACACAAAAA TTCCCTGCAAATCTCCCCCACCAGAACTAACAGACACTGCCACGAGCACAAAGAGATGCCCC AAGACACGTCCCAGGAAAGAAGTAAAAGAGGAGCTCTCAGCAGTTGAGAGGCTCACGCAAAC ATCAGGGCAAAGCACACACACACACAAAGAACCAGCAAGCGGTGATGAGGGCATCAAAGTAT TGAAGCAACGTGCAAAGAAGAAACCAAACCCAGTAGAAGAGGAACCCAGCAGGAGAAGGCCA AGAGCACCTAAGGAAAAGGCCCAACCCCTGGAAGACCTGGCCGGCTTCACAGAGCTCTCTGA AACATCAGGTCACACTCAGGAATCACTGACTGCTGGCAAAGCCACTAAAATACCCTGCGAAT CTCCCCCACTAGAAGTGGTAGACACCACAGCAAGCACAAAGAGGCATCTCAGGACACGTGTG CAGAAGGTACAAGTAAAAGAAGAGCCTTCAGCAGTCAAGTTCACACAAACATCAGGGGAAAC CACGGATGCAGACAAAGAACCAGCAGGTGAAGATAAAGGCATCAAAGCATTGAAGGAATCTG CAAAACAGACACCGGCTCCAGCAGCAAGTGTAACTGGCAGCAGGAGACGGCCAAGAGCACCC AGGGAAAGTGCCCAAGCCATAGAAGACCTAGCTGGCTTCAAAGACCCAGCAGCAGGTCACAC TGAAGAATCAATGACTGATGACAAAACCACTAAAATACCCTGCAAATCATCACCAGAACTAG AAGACACCGCAACAAGCTCAAAGAGACGGCCCAGGACACGTGCCCAGAAAGTAGAAGTGAAG GAGGAGCTGTTAGCAGTTGGCAAGCTCACACAAACCTCAGGGGAGACCACGCACACCGACAA AGAGCCGGTAGGTGAGGGCAAAGGCACGAAAGCATTTAAGCAACCTGCAAAGCGGAAGCTGG ACGCAGAAGATGTAATTGGCAGCAGGAGACAGCCAAGAGCACCTAAGGAAAAGGCCCAACCC CTGGAAGATCTGGCCAGCTTCCAAGAGCTCTCTCAAACACCAGGCCACACTGAGGAACTGGC AAATGGTGCTGCTGATAGCTTTACAAGCGCTCCAAAGCAAACACCTGACAGTGGAAAACCTC TAAAAATATCCAGAAGAGTTCTTCGGGCCCCTAAAGTAGAACCCGTGGGAGACGTGGTAAGC ACCAGAGACCCTGTAAAATCACAAAGCAAAAGCAACACTTCCCTGCCCCCACTGCCCTTCAA GAGGGGAGGTGGCAAAGATGGAAGCGTCACGGGAACCAAGAGGCTGCGCTGCATGCCAGCAC CAGAGGAAATTGTGGAGGAGCTGCCAGCCAGCAAGAAGCAGAGGGTTGCTCCCAGGGCAAGA GGCAAATCATCCGAACCCGTGGTCATCATGAAGAGAAGTTTGAGGACTTCTGCAAAAAGAAT TGAACCTGCGGAAGAGCTGAACAGCAACGACATGAAAACCAACAAAGAGGAACACAAATTAC AAGACTCGGTCCCTGAAAATAAGGGAATATCCCTGCGCTCCAGACGCCAAAATAAGACTGAG GCAGAACAGCAAATAACTGAGGTCTTTGTATTAGCAGAAAGAATAGAAATAAACAGAAATGA AAAGAAGCCCATGAAGACCTCCCCAGAGATGGACATTCAGAATCCAGATGATGGAGCCCGGA AACCCATACCTAGAGACAAAGTCACTGAGAACAAAAGGTGCTTGAGGTCTGCTAGACAGAAT GAGAGCTCCCAGCCTAAGGTGGCAGAGGAGAGCGGAGGGCAGAAGAGTGCGAAGGTTCTCAT GCAGAATCAGAAAGGGAAAGGAGAAGCAGGAAATTCAGACTCCATGTGCCTGAGATCAAGAA AGACAAAAAGCCAGCCTGCAGCAAGCACTTTGGAGAGCAAATCTGTGCAGAGAGTAACGCGG AGTGTCAAGAGGTGTGCAGAAAATCCAAAGAAGGCTGAGGACAATGTGTGTGTCAAGAAAAT AAGAACCAGAAGTCATAGGGACAGTGAAGATATTTGA (SEQ ID NO: 623). An exemplary Ki67 gene sequence is provided at ENSEMBL Accession No. ENSG00000148773 (SEQ ID NO: 624).

By “programmed cell death 1 (PD1) polypeptide” is meant a protein having at least about 85% amino acid sequence identity to GenBank Accession No. AJS10360.1, which is provided below, or a fragment thereof having immunomodulatory activity.

>AJS10360.1 programmed cell death 1 protein
[Homo sapiens]
(SEQ ID NO: 628)
MQIPQAPWPVVWAVLQLGWRPGWFLDSPDRPWNPPTFSPALLVVT
EGDNATFTCSFSNTSESFVLNWYRMSPSNQTDKLAAFPEDRSQPG
QDCRFRVTQLPNGRDFHMSVVRARRNDSGTYLCGAISLAPKAQIK
ESLRAELRVTERRAEVPTAHPSPSPRPAGQFQTLVVGVVGGLLGS
LVLLVWVLAVICSRAARGTIGARRTGQPLKEDPSAVPVFSVDYGE
LDFQWREKTPEPPVPCVPEQTEYATIVFPSGMGTSSPARRGSADG
PRSAQPLRPEDGHCSWPL.

By “programmed cell death 1 (PD1) polynucleotide” is meant a nucleic acid molecule encoding a PD1 polypeptide, as well as the introns, exons, 3′ untranslated regions, 5′ untranslated regions, and regulatory sequences associated with its expression, or fragments thereof. An exemplary PD1 polynucleotide sequence is provided at GenBank Accession No. KJ865861.1, which is provided below.

>KJ865861.1 Homo sapiens Cell-Line G3361 Programmed Cell Death 1 Protein (PDCD1) mRNA, Complete Cds
ATGCAGATCCCACAGGCGCCCTGGCCAGTCGTCTGGGCGGTGCTACAACTGGGCTGGCGGCC AGGATGGTTCTTAGACTCCCCAGACAGGCCCTGGAACCCCCCCACCTTCTCCCCAGCCCTGC TCGTGGTGACCGAAGGGGACAACGCCACCTTCACCTGCAGCTTCTCCAACACATCGGAGAGC TTCGTGCTAAACTGGTACCGCATGAGCCCCAGCAACCAGACGGACAAGCTGGCCGCCTTCCC CGAGGACCGCAGCCAGCCCGGCCAGGACTGCCGCTTCCGTGTCACACAACTGCCCAACGGGC GTGACTTCCACATGAGCGTGGTCAGGGCCCGGCGCAATGACAGCGGCACCTACCTCTGTGGG GCCATCTCCCTGGCCCCCAAGGCGCAGATCAAAGAGAGCCTGCGGGCAGAGCTCAGGGTGAC AGAGAGAAGGGCAGAAGTGCCCACAGCCCACCCCAGCCCCTCACCCAGGCCAGCCGGCCAGT TCCAAACCCTGGTGGTTGGTGTCGTGGGCGGCCTGCTGGGCAGCCTGGTGCTGCTAGTCTGG GTCCTGGCCGTCATCTGCTCCCGGGCCGCACGAGGGACAATAGGAGCCAGGCGCACCGGCCA GCCCCTGAAGGAGGACCCCTCAGCCGTGCCTGTGTTCTCTGTGGACTATGGGGAGCTGGATT TCCAGTGGCGAGAGAAGACCCCGGAGCCCCCCGTGCCCTGTGTCCCTGAGCAGACGGAGTAT GCCACCATTGTCTTTCCTAGCGGAATGGGCACCTCATCCCCCGCCCGCAGGGGCTCAGCCGA CGGCCCTCGGAGTGCCCAGCCACTGAGGCCTGAGGATGGACACTGCTCTTGGCCCCTCTGA (SEQ ID NO: 629). An exemplary PD1 polynucleotide sequence is also provided at Ensenbl accession no: ENSG00000188389 (SEQ ID NO: 630).

By “Profilin 1 (PFN1) polypeptide” is meant a protein having at least about 85% amino acid sequence identity to GenBank Accession No. AAH02475.1, which is provided below, or a fragment thereof having immunomodulatory activity.

>AAH02475.1 Profilin 1
[Homo sapiens]
(SEQ ID NO: 631)
MAGWNAYIDNLMADGTCQDAAIVGYKDSPSVWAAVPGKTFVNITP
AEVGVLVGKDRSSFYVNGLTLGGQKCSVIRDSLLQDGEFSMDLRT
KSTGGAPTFNVTVTKTDKTLVLLMGKEGVHGGLINKKCYEMASHL
RRSQY.

By “Profilin 1 (PFN1) polynucleotide” is meant a polynucleotide encoding a PFN1 polypeptide, as well as the introns, exons, 3′ untranslated regions, 5′ untranslated regions, and regulatory sequences associated with its expression, or fragments thereof. In embodiments, a PFN1 polynucleotide is the genomic sequence, cDNA, mRNA, or gene associated with and/or required for PFN1 expression. An exemplary PFN1 nucleic acid sequence is provided below (GenBank Accession No. BC002475.2:123-545).

>BC002475.2:123-545 Homo sapiens Profilin 1, mRNA (cDNA Clone MGC:2264 IMAGE:3349355), Complete Cds
ATGGCCGGGTGGAACGCCTACATCGACAACCTCATGGCGGACGGGACCTGTCAGGACGCGGC CATCGTGGGCTACAAGGACTCGCCCTCCGTCTGGGCCGCCGTCCCCGGGAAAACGTTCGTCA ACATCACGCCAGCTGAGGTGGGTGTCCTGGTTGGCAAAGACCGGTCAAGTTTTTACGTGAAT GGGCTGACACTTGGGGGCCAGAAATGTTCGGTGATCCGGGACTCACTGCTGCAGGATGGGGA ATTTAGCATGGATCTTCGTACCAAGAGCACCGGTGGGGCCCCCACCTTCAATGTCACTGTCA CCAAGACTGACAAGACGCTAGTCCTGCTGATGGGCAAAGAAGGTGTCCACGGTGGTTTGATC AACAAGAAATGTTATGAAATGGCCTCCCACCTTCGGCGTTCCCAGTACTGA (SEQ ID NO: 632). An exemplary PFN1 gene sequence is provided at ENSEMBL Accession No. ENSG00000108518 (SEQ ID NO: 633).

By “PR domain-containing protein 1 (PRDM1) polypeptide” is meant a protein having at least about 85% amino acid sequence identity to GenBank Accession No. AAO45623.1, which is provided below, or a fragment thereof having immunomodulatory activity.

>AAO45623.1 PR domain-containing protein 1 alpha
[Homo sapiens]
(SEQ ID NO: 634)
MKMDMEDADMTLWTEAEFEEKCTYIVNDHPWDSGADGGTSVQAEA
SLPRNLLFKYATNSEEVIGVMSKEYIPKGTRFGPLIGEIYTNDTV
PKNANRKYFWRIYSRGELHHFIDGFNEEKSNWMRYVNPAHSPREQ
NLAACQNGMNIYFYTIKPIPANQELLVWYCRDFAERLHYPYPGEL
TMMNLTQTQSSLKQPSTEKNELCPKNVPKREYSVKEILKLDSNPS
KGKDLYRSNISPLTSEKDLDDFRRRGSPEMPFYPRVVYPIRAPLP
EDFLKASLAYGIERPTYITRSPIPSSTTPSPSARSSPDQSLKSSS
PHSSPGNTVSPVGPGSQEHRDSYAYLNASYGTEGLGSYPGYAPLP
HLPPAFIPSYNAHYPKFLLPPYGMNCNGLSAVSSMNGINNFGLFP
RLCPVYSNLLGGGSLPHPMLNPTSLPSSLPSDGARRLLQPEHPRE
VLVPAPHSAFSFTGAAASMKDKACSPTSGSPTAGTAATAEHVVQP
KATSAAMAAPSSDEAMNLIKNKRNMTGYKTLPYPLKKQNGKIKYE
CNVCAKTFGQLSNLKVHLRVHSGERPFKCQTCNKGFTQLAHLQKH
YLVHTGEKPHECQVCHKRFSSTSNLKTHLRLHSGEKPYQCKVCPA
KFTQFVHLKLHKRLHTRERPHKCSQCHKNYIHLCSLKVHLKGNCA
AAPAPGLPLEDLTRINEEIEKFDISDNADRLEDVEDDISVISVVE
KEILAVVRKEKEETGLKVSLQRNMGNGLLSSGCSLYESSDLPLMK
LPPSNPLPLVPVKVKQETVEPMDP.

By “PR domain-containing protein 1 (PRDM1) polynucleotide” is meant a polynucleotide encoding a PRDM1 polypeptide, as well as the introns, exons, 3′ untranslated regions, 5′ untranslated regions, and regulatory sequences associated with its expression, or fragments thereof. In embodiments, a PRDM1 polynucleotide is the genomic sequence, cDNA, mRNA, or gene associated with and/or required for PRDM1 expression. An exemplary PRDM1 nucleic acid sequence is provided below (GenBank Accession No. AY198414.1:343-2712).

>AY198414.1:343-2712 Homo sapiens PR Domain-Containing Protein 1 Alpha (PRDM1) mRNA, Complete Cds
ATGAAAATGGACATGGAGGATGCGGATATGACTCTGTGGACAGAGGCTGAGTTTGAAGAGAA GTGTACATACATTGTGAACGACCACCCCTGGGATTCTGGTGCTGATGGCGGTACTTCGGTTC AGGCGGAGGCATCCTTACCAAGGAATCTGCTTTTCAAGTATGCCACCAACAGTGAAGAGGTT ATTGGAGTGATGAGTAAAGAATACATACCAAAGGGCACACGTTTTGGACCCCTAATAGGTGA AATCTACACCAATGACACAGTTCCTAAGAACGCCAACAGGAAATATTTTTGGAGGATCTATT CCAGAGGGGAGCTTCACCACTTCATTGACGGCTTTAATGAAGAGAAAAGCAACTGGATGCGC TATGTGAATCCAGCACACTCTCCCCGGGAGCAAAACCTGGCTGCGTGTCAGAACGGGATGAA CATCTACTTCTACACCATTAAGCCCATCCCTGCCAACCAGGAACTTCTTGTGTGGTATTGTC GGGACTTTGCAGAAAGGCTTCACTACCCTTATCCCGGAGAGCTGACAATGATGAATCTCACA CAAACACAGAGCAGTCTAAAGCAACCGAGCACTGAGAAAAATGAACTCTGCCCAAAGAATGT CCCAAAGAGAGAGTACAGCGTGAAAGAAATCCTAAAATTGGACTCCAACCCCTCCAAAGGAA AGGACCTCTACCGTTCTAACATTTCACCCCTCACATCAGAAAAGGACCTCGATGACTTTAGA AGACGTGGGAGCCCCGAAATGCCCTTCTACCCTCGGGTCGTTTACCCCATCCGGGCCCCTCT GCCAGAAGACTTTTTGAAAGCTTCCCTGGCCTACGGGATCGAGAGACCCACGTACATCACTC GCTCCCCCATTCCATCCTCCACCACTCCAAGCCCCTCTGCAAGAAGCAGCCCCGACCAAAGC CTCAAGAGCTCCAGCCCTCACAGCAGCCCTGGGAATACGGTGTCCCCTGTGGGCCCCGGCTC TCAAGAGCACCGGGACTCCTACGCTTACTTGAACGCGTCCTACGGCACGGAAGGTTTGGGCT CCTACCCTGGCTACGCACCCCTGCCCCACCTCCCGCCAGCTTTCATCCCCTCGTACAACGCT CACTACCCCAAGTTCCTCTTGCCCCCCTACGGCATGAATTGTAATGGCCTGAGCGCTGTGAG CAGCATGAATGGCATCAACAACTTTGGCCTCTTCCCGAGGCTGTGCCCTGTCTACAGCAATC TCCTCGGTGGGGGCAGCCTGCCCCACCCCATGCTCAACCCCACTTCTCTCCCGAGCTCGCTG CCCTCAGATGGAGCCCGGAGGTTGCTCCAGCCGGAGCATCCCAGGGAGGTGCTTGTCCCGGC GCCCCACAGTGCCTTCTCCTTTACCGGGGCCGCCGCCAGCATGAAGGACAAGGCCTGTAGCC CCACAAGCGGGTCTCCCACGGCGGGAACAGCCGCCACGGCAGAACATGTGGTGCAGCCCAAA GCTACCTCAGCAGCGATGGCAGCCCCCAGCAGCGACGAAGCCATGAATCTCATTAAAAACAA AAGAAACATGACCGGCTACAAGACCCTTCCCTACCCGCTGAAGAAGCAGAACGGCAAGATCA AGTACGAATGCAACGTTTGCGCCAAGACTTTCGGCCAGCTCTCCAATCTGAAGGTCCACCTG AGAGTGCACAGTGGAGAACGGCCTTTCAAATGTCAGACTTGCAACAAGGGCTTTACTCAGCT CGCCCACCTGCAGAAACACTACCTGGTACACACGGGAGAAAAGCCACATGAATGCCAGGTCT GCCACAAGAGATTTAGCAGCACCAGCAATCTCAAGACCCACCTGCGACTCCATTCTGGAGAG AAACCATACCAATGCAAGGTGTGCCCTGCCAAGTTCACCCAGTTTGTGCACCTGAAACTGCA CAAGCGTCTGCACACCCGGGAGCGGCCCCACAAGTGCTCCCAGTGCCACAAGAACTACATCC ATCTCTGTAGCCTCAAGGTTCACCTGAAAGGGAACTGCGCTGCGGCCCCGGCGCCTGGGCTG CCCTTGGAAGATCTGACCCGAATCAATGAAGAAATCGAGAAGTTTGACATCAGTGACAATGC TGACCGGCTCGAGGACGTGGAGGATGACATCAGTGTGATCTCTGTAGTGGAGAAGGAAATTC TGGCCGTGGTCAGAAAAGAGAAAGAAGAAACTGGCCTGAAAGTGTCTTTGCAAAGAAACATG GGGAATGGACTCCTCTCCTCAGGGTGCAGCCTTTATGAGTCATCAGATCTACCCCTCATGAA GTTGCCTCCCAGCAACCCACTACCTCTGGTACCTGTAAAGGTCAAACAAGAAACAGTTGAAC CAATGGATCCTTAA (SEQ ID NO: 635). An exemplary PRDM1 gene sequence is provided at ENSEMBL Accession No. ENSG00000057657 (SEQ ID NO: 636).

By “Protein kinase, cAMP-dependent, catalytic, alpha (PRKACA) polypeptide” is meant a protein having at least about 85% amino acid sequence identity to GenBank Accession No. AAH39846.1, which is provided below, or a fragment thereof having immunomodulatory activity.

>AAH39846.1 Protein kinase, cAMP-dependent,
catalytic, alpha[Homo sapiens]
(SEQ ID NO: 637)
MGNAAAAKKGSEQESVKEFLAKAKEDFLKKWESPAQNTAHLDQFE
RIKTIGTGSFGRVMLVKHKETGNHYAMKILDKQKVVKLKQIEHTL
NEKRILQAVNFPFLVKLEFSFKDNSNLYMVMEYVPGGEMFSHLRR
IGRFSEPHARFYAAQIVLTFEYLHSLDLIYRDLKPENLLIDQQGY
IQVTDFGFAKRVKGRTWTLCGTPEYLAPEIILSKGYNKAVDWWAL
GVLIYEMAAGYPPFFADQPIQIYEKIVSGKVRFPSHFSSDLKDLL
RNLLQVDLTKRFGNLKNGVNDIKNHKWFATTDWIAIYQRKVEAPF
IPKFKGPGDTSNFDDYEEEEIRVSINEKCGKEFSEF.

By “Protein kinase, cAMP-dependent, catalytic, alpha (PRKACA) polynucleotide” is meant a polynucleotide encoding a PRKACA polypeptide, as well as the introns, exons, 3′ untranslated regions, 5′ untranslated regions, and regulatory sequences associated with its expression, or fragments thereof. In embodiments, a PRKACA polynucleotide is the genomic sequence, cDNA, mRNA, or gene associated with and/or required for PRKACA expression. An exemplary PRKACA nucleic acid sequence is provided below (GenBank Accession No. BC039846.1:197-1252).

>BC039846.1:197-1252 Homo sapiens Protein Kinase, cAMP-Dependent, Catalytic, Alpha, mRNA (cDNA Clone MGC:48865 IMAGE:6066764), Complete Cds
ATGGGCAACGCCGCCGCCGCCAAGAAGGGCAGCGAGCAGGAGAGCGTGAAAGAATTCTTAGC CAAAGCCAAAGAAGATTTTCTTAAAAAATGGGAAAGTCCCGCTCAGAACACAGCCCACTTGG ATCAGTTTGAACGAATCAAGACCCTCGGCACGGGCTCCTTCGGGCGGGTGATGCTGGTGAAA CACAAGGAGACCGGGAACCACTATGCCATGAAGATCCTCGACAAACAGAAGGTGGTGAAACT GAAACAGATCGAACACACCCTGAATGAAAAGCGCATCCTGCAAGCTGTCAACTTTCCGTTCC TCGTCAAACTCGAGTTCTCCTTCAAGGACAACTCAAACTTATACATGGTCATGGAGTACGTG CCCGGCGGGGAGATGTTCTCACACCTACGGCGGATCGGAAGGTTCAGTGAGCCCCATGCCCG TTTCTACGCGGCCCAGATCGTCCTGACCTTTGAGTATCTGCACTCGCTGGATCTCATCTACA GGGACCTGAAGCCGGAGAATCTGCTCATTGACCAGCAGGGCTACATTCAGGTGACAGACTTC GGTTTCGCCAAGCGCGTGAAGGGCCGCACTTGGACCTTGTGCGGCACCCCTGAGTACCTGGC CCCTGAGATTATCCTGAGCAAAGGCTACAACAAGGCCGTGGACTGGTGGGCCCTGGGGGTTC TTATCTATGAAATGGCCGCTGGCTACCCGCCCTTCTTCGCAGACCAGCCCATCCAGATCTAT GAGAAGATCGTCTCTGGGAAGGTGCGCTTCCCTTCCCACTTCAGCTCTGACTTGAAGGACCT GCTGCGGAACCTCCTGCAGGTAGATCTCACCAAGCGCTTTGGGAACCTCAAGAATGGGGTCA ACGATATCAAGAACCACAAGTGGTTTGCCACAACTGACTGGATTGCCATCTACCAGAGGAAG GTGGAAGCTCCCTTCATACCAAAGTTTAAAGGCCCTGGGGATACGAGTAACTTTGACGACTA TGAGGAAGAAGAAATCCGGGTCTCCATCAATGAGAAGTGTGGCAAGGAGTTTTCTGAGTTTT AG (SEQ ID NO: 638). An exemplary PRKACA gene sequence is provided at ENSEMBL Accession No. ENSG00000072062 (SEQ ID NO: 639).

By “Protein tyrosine phosphatase, nonreceptor-type, 1B (PTP1B) polypeptide” is meant a protein having at least about 85% amino acid sequence identity to GenBank Accession No. AAA60157.1, which is provided below, or a fragment thereof having immunomodulatory activity.

>AAA60157.1 non-receptor tyrosine phosphatase 1
[Homo sapiens]
(SEQ ID NO: 640)
MEMEKEFEQIDKSGSWAAIYQDIRHEASDFPCRVAKLPKNKNRNR
YRDVSPFDHSRIKLHQEDNDYINASLIKMEEAQRSYILTQGPLPN
TCGHFWEMVWEQKSRGVVMLNRVMEKGSLKCAQYWPQKEEKEMIF
EDTNLKLTLISEDIKSYYTVRQLELENLTTQETREILHFHYTTWP
DFGVPESPASFLNFLFKVRESGSLSPEHGPVVVHCSAGIGRSGTF
CLADTCLLLMDKRKDPSSVDIKKVLLEMRKFRMGLIQTADQLRFS
YLAVIEGAKFIMGDSSVQDQWKELSHEDLEPPPEHIPPPPRPPKR
ILEPHNGKCREFFPNHQWVKEETQEDKDCPIKEEKGSPLNAAPYG
IESMSQDTEVRSRVVGGSLRGAQAASPAKGEPSLPEKDEDHALSY
WKPFLVNMCVATVLTAGAYLCYRFLENSNT.

By “Protein tyrosine phosphatase, nonreceptor-type, 1B (PTP1B) polynucleotide” is meant a polynucleotide encoding a PTP1B polypeptide, as well as the introns, exons, 3′ untranslated regions, 5′ untranslated regions, and regulatory sequences associated with its expression, or fragments thereof. In embodiments, a PTP1B polynucleotide is the genomic sequence, cDNA, mRNA, or gene associated with and/or required for PTP1B expression. An exemplary PTP1B nucleic acid sequence is provided below (GenBank Accession No. M33689.1:73-1380).

>M33689.1:73-1380 Human (Clone Lambda-16-1) Non-Receptor Tyrosine Phosphatase 1 (PTPN1) mRNA, Complete Cds
ATGGAGATGGAAAAGGAGTTCGAGCAGATCGACAAGTCCGGGAGCTGGGCGGCCATTTACCA GGATATCCGACATGAAGCCAGTGACTTCCCATGTAGAGTGGCCAAGCTTCCTAAGAACAAAA ACCGAAATAGGTACAGAGACGTCAGTCCCTTTGACCATAGTCGGATTAAACTACATCAAGAA GATAATGACTATATCAACGCTAGTTTGATAAAAATGGAAGAAGCCCAAAGGAGTTACATTCT TACCCAGGGCCCTTTGCCTAACACATGCGGTCACTTTTGGGAGATGGTGTGGGAGCAGAAAA GCAGGGGTGTCGTCATGCTCAACAGAGTGATGGAGAAAGGTTCGTTAAAATGCGCACAATAC TGGCCACAAAAAGAAGAAAAAGAGATGATCTTTGAAGACACAAATTTGAAATTAACATTGAT CTCTGAAGATATCAAGTCATATTATACAGTGCGACAGCTAGAATTGGAAAACCTTACAACCC AAGAAACTCGAGAGATCTTACATTTCCACTATACCACATGGCCTGACTTTGGAGTCCCTGAA TCACCAGCCTCATTCTTGAACTTTCTTTTCAAAGTCCGAGAGTCAGGGTCACTCAGCCCGGA GCACGGGCCCGTTGTGGTGCACTGCAGTGCAGGCATCGGCAGGTCTGGAACCTTCTGTCTGG CTGATACCTGCCTCTTGCTGATGGACAAGAGGAAAGACCCTTCTTCCGTTGATATCAAGAAA GTGCTGTTAGAAATGAGGAAGTTTCGGATGGGGCTGATCCAGACAGCCGACCAGCTGCGCTT CTCCTACCTGGCTGTGATCGAAGGTGCCAAATTCATCATGGGGGACTCTTCCGTGCAGGATC AGTGGAAGGAGCTTTCCCACGAGGACCTGGAGCCCCCACCCGAGCATATCCCCCCACCTCCC CGGCCACCCAAACGAATCCTGGAGCCACACAATGGGAAATGCAGGGAGTTCTTCCCAAATCA CCAGTGGGTGAAGGAAGAGACCCAGGAGGATAAAGACTGCCCCATCAAGGAAGAAAAAGGAA GCCCCTTAAATGCCGCACCCTACGGCATCGAAAGCATGAGTCAAGACACTGAAGTTAGAAGT CGGGTCGTGGGGGGAAGTCTTCGAGGTGCCCAGGCTGCCTCCCCAGCCAAAGGGGAGCCGTC ACTGCCCGAGAAGGACGAGGACCATGCACTGAGTTACTGGAAGCCCTTCCTGGTCAACATGT GCGTGGCTACGGTCCTCACGGCCGGCGCTTACCTCTGCTACAGGTTCCTGTTCAACAGCAAC ACATAG (SEQ ID NO: 641). An exemplary PTP1B gene sequence is provided at ENSEMBL Accession No. ENSG00000196396 (SEQ ID NO: 642).

By “Protein-tyrosine phosphatase, nonreceptor-type 6, (PTPN6) polypeptide” is meant a protein having at least about 85% amino acid sequence identity to GenBank Accession No. AAH02523.1, which is provided below, or a fragment thereof having immunomodulatory activity.

>AAH02523.1 Protein tyrosine phosphatase,
non-receptor type 6 [Homo sapiens]
(SEQ ID NO: 643)
MLSRGWFHRDLSGLDAETLLKGRGVHGSFLARPSRKNQGDFSLSV
RVGDQVTHIRIQNSGDFYDLYGGEKFATLTELVEYYTQQQGVLQD
RDGTIIHLKYPLNCSDPTSERWYHGHMSGGQAETLLQAKGEPWTF
LVRESLSQPGDFVLSVLSDQPKAGPGSPLRVTHIKVMCEGGRYTV
GGLETFDSLTDLVEHFKKTGIEEASGAFVYLRQPYYATRVNAADI
ENRVLELNKKQESEDTAKAGFWEEFESLQKQEVKNLHQRLEGQRP
ENKGKNRYKNILPFDHSRVILQGRDSNIPGSDYINANYIKNQLLG
PDENAKTYIASQGCLEATVNDFWQMAWQENSRVIVMTTREVEKGR
NKCVPYWPEVGMQRAYGPYSVTNCGEHDTTEYKLRTLQVSPLDNG
DLIREIWHYQYLSWPDHGVPSEPGGVLSFLDQINQRQESLPHAGP
IIVHCSAGIGRTGTIIVIDMLMENISTKGLDCDIDIQKTIQMVRA
QRSGMVQTEAQYKFIYVAIAQFIETTKKKLEVLQSQKGQESEYGN
ITYPPAMKNAHAKASRTSSKHKEDVYENLHTKNKREEKVKKQRSA
DKEKSKGSLKRK.

By “Protein-tyrosine phosphatase, nonreceptor-type 6, (PTPN6) polynucleotide” is meant a polynucleotide encoding a PTPN6 polypeptide, as well as the introns, exons, 3′ untranslated regions, 5′ untranslated regions, and regulatory sequences associated with its expression, or fragments thereof. In embodiments, a PTPN6 polynucleotide is the genomic sequence, cDNA, mRNA, or gene associated with and/or required for PTPN6 expression. An exemplary PTPN6 nucleic acid sequence is provided below (GenBank Accession No. BC002523.2:136-1929).

>BC002523.2:136-1929 Homo sapiens Protein Tyrosine Phosphatase, Non-Receptor Type 6, mRNA (cDNA Clone MGC:2332 IMAGE:3140265), Complete Cds
ATGCTGTCCCGTGGGTGGTTTCACCGAGACCTCAGTGGGCTGGATGCAGAGACCCTGCTCAA GGGCCGAGGTGTCCACGGTAGCTTCCTGGCTCGGCCCAGTCGCAAGAACCAGGGTGACTTCT CGCTCTCCGTCAGGGTGGGGGATCAGGTGACCCATATTCGGATCCAGAACTCAGGGGATTTC TATGACCTGTATGGAGGGGAGAAGTTTGCGACTCTGACAGAGCTGGTGGAGTACTACACTCA GCAGCAGGGTGTCCTGCAGGACCGCGACGGCACCATCATCCACCTCAAGTACCCGCTGAACT GCTCCGATCCCACTAGTGAGAGGTGGTACCATGGCCACATGTCTGGCGGGCAGGCAGAGACG CTGCTGCAGGCCAAGGGCGAGCCCTGGACGTTTCTTGTGCGTGAGAGCCTCAGCCAGCCTGG AGACTTCGTGCTTTCTGTGCTCAGTGACCAGCCCAAGGCTGGCCCAGGCTCCCCGCTCAGGG TCACCCACATCAAGGTCATGTGCGAGGGTGGACGCTACACAGTGGGTGGTTTGGAGACCTTC GACAGCCTCACGGACCTGGTGGAGCATTTCAAGAAGACGGGGATTGAGGAGGCCTCAGGCGC CTTTGTCTACCTGCGGCAGCCGTACTATGCCACGAGGGTGAATGCGGCTGACATTGAGAACC GAGTGTTGGAACTGAACAAGAAGCAGGAGTCCGAGGATACAGCCAAGGCTGGCTTCTGGGAG GAGTTTGAGAGTTTGCAGAAGCAGGAGGTGAAGAACTTGCACCAGCGTCTGGAAGGGCAGCG GCCAGAGAACAAGGGCAAGAACCGCTACAAGAACATTCTCCCCTTTGACCACAGCCGAGTGA TCCTGCAGGGACGGGACAGTAACATCCCCGGGTCCGACTACATCAATGCCAACTACATCAAG AACCAGCTGCTAGGCCCTGATGAGAACGCTAAGACCTACATCGCCAGCCAGGGCTGTCTGGA GGCCACGGTCAATGACTTCTGGCAGATGGCGTGGCAGGAGAACAGCCGTGTCATCGTCATGA CCACCCGAGAGGTGGAGAAAGGCCGGAACAAATGCGTCCCATACTGGCCCGAGGTGGGCATG CAGCGTGCTTATGGGCCCTACTCTGTGACCAACTGCGGGGAGCATGACACAACCGAATACAA ACTCCGTACCTTACAGGTCTCCCCGCTGGACAATGGAGACCTGATTCGGGAGATCTGGCATT ACCAGTACCTGAGCTGGCCCGACCATGGGGTCCCCAGTGAGCCTGGGGGTGTCCTCAGCTTC CTGGACCAGATCAACCAGCGGCAGGAAAGTCTGCCTCACGCAGGGCCCATCATCGTGCACTG CAGCGCCGGCATCGGCCGCACAGGCACCATCATTGTCATCGACATGCTCATGGAGAACATCT CCACCAAGGGCCTGGACTGTGACATTGACATCCAGAAGACCATCCAGATGGTGCGGGCGCAG CGCTCGGGCATGGTGCAGACGGAGGCGCAGTACAAGTTCATCTACGTGGCCATCGCCCAGTT CATTGAAACCACTAAGAAGAAGCTGGAGGTCCTGCAGTCGCAGAAGGGCCAGGAGTCGGAGT ACGGGAACATCACCTATCCCCCAGCCATGAAGAATGCCCATGCCAAGGCCTCCCGCACCTCG TCCAAACACAAGGAGGATGTGTATGAGAACCTGCACACTAAGAACAAGAGGGAGGAGAAAGT GAAGAAGCAGCGGTCAGCAGACAAGGAGAAGAGCAAGGGTTCCCTCAAGAGGAAGTGA (SEQ ID NO: 644). An exemplary PTPN6 gene sequence is provided at ENSEMBL Accession No. ENSG00000111679 (SEQ ID NO: 645).

By “RAS p21 protein activator 2 (RASA2) polypeptide” is meant a protein having at least about 85% amino acid sequence identity to GenBank Accession No. EAW78998.1, which is provided below, or a fragment thereof having immunomodulatory activity.

>EAW78998.1 RAS p21 protein activator 2,
isoform CRA_a[Homo sapiens]
(SEQ ID NO: 646)
MAAAAPAAAAASSEAPAASATAEPEAGDQDSREVRVLQSLRGKIC
EAKNLLPYLGPHKMRDCFCTINLDQEEVYRTQVVEKSLSPFFSEE
FYFEIPRTFQYLSFYVYDKNVLQRDLRIGKVAIKKEDLCNHSGKE
TWFSLQPVDSNSEVQGKVHLELKLNELITENGTVCQQLVVHIKAC
HGLPLINGQSCDPYATVSLVGPSRNDQKKTKVKKKTSNPQFNEIF
YFEVTRSSSYTRKSQFQVEEEDIEKLEIRIDLWNNGNLVQDVFLG
EIKVPVNVLRTDSSHQAWYLLQPRDNGNKSSKTDDLGSLRLNICY
TEDYVLPSEYYGPLKTLLLKSPDVQPISASAAYILSEICRDKNDA
VLPLVRLLLHHDKLVPFATAVAELDLKDTQDANTIFRGNSLATRC
LDEMMKIVGGHYLKVTLKPILDEICDSSKSCEIDPIKLKEGDNVE
NNKENLRYYVDKLFNTIVKSSMSCPTVMCDIFYSLRQMATQRFPN
DPHVQYSAVSSFVFLRFFAVAVVSPHTFHLRPHHPDAQTIRTLTL
ISKTIQTLGSWGSLSKSKSSFKETFMCEFFKMFQEEGYIIAVKKF
LDEISSTETKESSGTSEPVHLKEGEMYKRAQGRTRIGKKNFKKRW
FCLTSRELTYHKQPGKDAIYTIPVKNILAVEKLEESSFNKKNMFQ
VIHTEKPLYVQANNCVEANEWIDVLCRVSRCNQNRLSFYHPSVYL
NGNWLCCQETGENTLGCKPCTAGVPADIQIDIDEDRETERIYSLF
TLSLLKLQKMEEACGTIAVYQGPQKEPDDYSNFVIEDSVTTFKTI
QQIKSIIEKLDEPHEKYRKKRSSSAKYGSKENPIVGKAS.

By “RAS p21 protein activator 2 (RASA2) polynucleotide” is meant a polynucleotide encoding a RASA2 polypeptide, as well as the introns, exons, 3′ untranslated regions, 5′ untranslated regions, and regulatory sequences associated with its expression, or fragments thereof. In embodiments, a RASA2 polynucleotide is the genomic sequence, cDNA, mRNA, or gene associated with and/or required for RASA2 expression. An exemplary RASA2 nucleic acid sequence is provided below (GenBank Accession No. CH471052.2:47815149-47815281,47840228-47840345,47844394-47844497,47857775-47857869,47868600-47868676,47881926-47882009,47883909-47883981,47886955-47887031,47887963-47888064,47898869-47899025,47899363-47899511,47900568-47900682,47901106-47901180,47901903-47902026,47904959-47905065,47908327-47908410,47909059-47909136,47913984-47914057,47914605-47914711,47935981-47936063,47936792-47937000,47937723-47937826,47938180-47938369,47940547-47940577).

>CH471052.2:47815149-47815281,47840228-47840345,47844394-47844497,47857775-47857869,47868600-47868676,47881926-47882009,47883909-47883981,47886955-47887031,47887963-47888064,47898869-47899025,47899363-47899511,47900568-47900682,47901106-47901180,47901903-47902026,47904959-47905065,47908327-47908410,47909059-47909136,47913984-47914057,47914605-47914711,47935981-47936063,47936792-47937000,47937723-47937826,47938180-47938369,47940547-47940577 Homo sapiens 211000035835664 Genomic Scaffold, Whole Genome Shotgun Sequence
ATGGCGGCGGCGGCGCCTGCTGCTGCGGCGGCTTCTTCCGAGGCGCCAGCGGCGAGTGCGAC TGCAGAGCCCGAGGCCGGGGACCAGGACAGTCGCGAGGTTCGAGTGTTGCAGAGCCTGCGGG GCAAGATCTGTGAAGCAAAAAATTTATTGCCATATCTTGGACCCCACAAAATGAGAGATTGT TTCTGTACCATAAATTTGGACCAGGAAGAAGTTTATCGTACCCAAGTTGTGGAAAAATCTTT AAGCCCATTTTTCAGTGAAGAATTTTACTTTGAGATTCCAAGAACTTTCCAGTATTTGTCTT TCTATGTTTATGATAAGAATGTTTTACAAAGAGATCTCCGTATAGGAAAAGTAGCCATCAAA AAAGAAGACTTGTGTAATCACAGTGGCAAAGAAACTTGGTTTTCATTACAGCCTGTTGACTC CAATTCAGAGGTTCAGGGTAAAGTTCACCTTGAATTAAAACTGAATGAACTGATAACGGAGA ATGGAACTGTATGCCAGCAGCTTGTTGTACACATCAAGGCATGCCATGGGTTGCCTCTCATA AATGGCCAAAGCTGTGACCCTTATGCAACAGTTTCTCTAGTGGGCCCTTCTAGGAATGACCA AAAGAAGACAAAAGTAAAGAAGAAAACAAGCAATCCGCAGTTTAATGAAATCTTTTATTTTG AGGTAACCAGATCCAGTAGTTACACCAGAAAGTCCCAGTTCCAGGTAGAAGAGGAGGACATT GAAAAGCTAGAAATCAGGATCGACTTGTGGAACAATGGAAACCTAGTCCAAGATGTTTTCCT AGGTGAGATTAAGGTTCCTGTGAACGTATTAAGAACTGATTCCTCTCATCAAGCCTGGTACT TGCTACAGCCAAGAGACAATGGAAACAAGTCATCCAAAACTGATGACCTGGGGTCTCTTCGA TTAAATATATGTTATACAGAAGACTACGTGCTTCCTTCAGAGTACTATGGTCCTTTGAAAAC TTTGCTGCTAAAATCACCAGATGTTCAACCAATATCTGCCTCAGCTGCTTACATTTTGAGTG AAATATGTCGAGATAAAAATGATGCTGTTTTGCCCCTTGTACGACTGCTGCTGCACCATGAT AAACTTGTTCCTTTTGCCACTGCTGTGGCTGAATTAGACTTGAAGGATACACAAGATGCAAA CACAATTTTTAGAGGAAATTCCCTGGCTACCCGATGTCTGGATGAGATGATGAAAATAGTGG GAGGGCACTACCTGAAAGTAACATTAAAACCTATTCTTGATGAGATATGTGACTCCTCAAAA TCCTGTGAAATCGATCCTATTAAATTGAAAGAGGGAGATAATGTAGAAAATAATAAGGAGAA TCTGCGCTACTATGTAGACAAGTTATTCAATACAATTGTAAAATCAAGTATGAGCTGCCCCA CTGTAATGTGTGATATCTTTTATTCTCTAAGGCAGATGGCTACTCAGAGATTTCCTAATGAC CCTCATGTTCAGTATTCTGCAGTGAGCAGCTTTGTATTTCTTCGTTTCTTTGCTGTAGCCGT AGTATCACCTCATACTTTTCATTTGCGACCTCATCATCCAGATGCACAGACAATTAGAACAT TAACTCTCATCTCAAAAACTATACAAACTTTGGGAAGCTGGGGGAGTCTGTCCAAAAGCAAG TCAAGTTTCAAAGAGACATTCATGTGTGAATTTTTCAAAATGTTTCAAGAAGAAGGATATAT TATAGCAGTTAAAAAGTTCTTGGATGAAATTTCATCTACTGAAACTAAAGAGTCCAGTGGTA CGAGTGAGCCTGTGCACCTGAAAGAAGGTGAGATGTATAAAAGAGCTCAAGGAAGAACTCGG ATTGGAAAAAAGAATTTTAAGAAACGATGGTTCTGCTTAACAAGCAGAGAGCTCACCTACCA CAAACAGCCAGGCAAAGATGCAATCTACACAATCCCAGTAAAAAACATTCTTGCTGTGGAAA AACTGGAAGAGAGCTCTTTCAACAAGAAAAATATGTTCCAAGTAATACATACGGAGAAACCA CTCTATGTCCAGGCAAATAACTGTGTAGAAGCTAATGAATGGATAGACGTACTCTGCAGGGT GAGCCGATGCAATCAAAACAGGCTCAGTTTTTATCATCCCTCTGTGTATCTAAACGGAAATT GGCTCTGCTGTCAGGAGACTGGTGAAAACACTCTCGGCTGCAAGCCATGTACTGCAGGTGTC CCTGCAGACATCCAAATAGATATTGATGAAGACAGAGAAACAGAAAGAATTTATTCCCTTTT TACCCTCAGTTTACTTAAGCTGCAGAAGATGGAAGAGGCTTGTGGAACTATTGCAGTCTATC AAGGACCACAGAAAGAGCCTGATGATTATTCTAACTTTGTAATCGAGGATTCTGTAACAACC TTTAAGACAATTCAGCAAATAAAAAGCATAATTGAGAAGCTGGATGAACCTCATGAAAAATA TAGGAAGAAAAGATCCAGTAGTGCAAAATATGGGAGCAAGGAAAATCCAATTGTTGGGAAAG CATCTTAG (SEQ ID NO: 647). An exemplary RASA2 gene sequence is provided at ENSEMBL Accession No. ENSG00000155903 (SEQ ID NO: 648).

By “Zinc finger CCCH domain-containing protein 12A (Regnase-1) polypeptide” is meant a protein having at least about 85% amino acid sequence identity to GenBank Accession No. AAH05001.1, which is provided below, or a fragment thereof having immunomodulatory activity.

>AAH05001.1 Zinc finger CCCH-type containing
12A [Homo sapiens]
(SEQ ID NO: 649)
MSGPCGEKPVLEASPTMSLWEFEDSHSRQGTPRPGQELAAEEASA
LELQMKVDFFRKLGYSSTEIHSVLQKLGVQADTNTVLGELVKHGT
ATERERQTSPDPCPQLPLVPRGGGTPKAPNLEPPLPEEEKEGSDL
RPVVIDGSNVAMSHGNKEVFSCRGILLAVNWFLERGHTDITVFVP
SWRKEQPRPDVPITDQHILRELEKKKILVFTPSRRVGGKRVVCYD
DRFIVKLAYESDGIVVSNDTYRDLQGERQEWKRFIEERLLMYSFV
NDKFMPPDDPLGRHGPSLDNFLRKKPLTLEHRKQPCPYGRKCTYG
IKCRFFHPERPSCPQRSVADELRANALLSPPRAPSKDKNGRRPSP
SSQSSSLLTESEQCSLDGKKLGAQASPGSRQEGLTQTYAPSGRSL
APSGGSGSSFGPTDWLPQTLDSLPYVSQDCLDSGIGSLESQMSEL
WGVRGGGPGEPGPPRAPYTGYSPYGSELPATAAFSAFGRAMGAGH
FSVPADYPPAPPAFPPREYWSEPYPLPPPTSVLQEPPVQSPGAGR
SPWGRADSLAKEQASVYTKLCGVFPPHLVEAVMGRFPQLLDPQQL
AAEILSYKSQHPSE.

By “Zinc finger CCCH domain-containing protein 12A (Regnase-1) polynucleotide” is meant a polynucleotide encoding a Regnase-1 polypeptide, as well as the introns, exons, 3′ untranslated regions, 5′ untranslated regions, and regulatory sequences associated with its expression, or fragments thereof. In embodiments, a Regnase-1 polynucleotide is the genomic sequence, cDNA, mRNA, or gene associated with and/or required for Regnase-1 expression. An exemplary Regnase-1 nucleic acid sequence is provided below (GenBank Accession No. BC0005001.1:75-1874).

>BC0005001.1:75-1874 Homo sapiens Zinc Finger CCCH-Type Containing 12A, mRNA (cDNA Clone MGC:4427 IMAGE:2958199), Complete Cds
ATGAGTGGCCCCTGTGGAGAGAAGCCTGTCCTGGAAGCCAGCCCCACCATGAGTCTGTGGGA ATTTGAGGACAGCCACAGCCGTCAGGGCACCCCAAGGCCGGGTCAAGAGCTGGCCGCTGAGG AGGCCTCGGCCCTGGAACTGCAGATGAAGGTGGACTTCTTCCGGAAGCTGGGCTATTCATCC ACGGAGATCCACAGCGTCCTGCAGAAGCTGGGCGTCCAGGCAGACACCAACACGGTGCTGGG TGAGCTGGTGAAACACGGGACAGCCACCGAGCGGGAGCGCCAGACCTCACCGGACCCCTGCC CTCAGCTCCCTCTAGTCCCGCGGGGTGGTGGCACCCCTAAGGCTCCCAACCTGGAGCCTCCA CTCCCAGAAGAGGAAAAGGAGGGCAGCGACCTGAGACCAGTGGTCATCGATGGGAGCAACGT GGCCATGAGCCATGGGAACAAGGAGGTCTTCTCCTGCCGGGGCATCCTGCTGGCAGTGAACT GGTTTCTGGAGCGGGGCCACACAGACATCACAGTGTTTGTGCCATCCTGGAGGAAGGAGCAG CCTCGGCCCGACGTGCCCATCACAGACCAGCACATCCTGCGGGAACTGGAGAAGAAGAAGAT CCTGGTGTTCACACCATCACGACGCGTGGGTGGCAAGCGGGTGGTGTGCTATGACGACAGAT TCATTGTGAAGCTGGCCTACGAGTCTGACGGGATCGTGGTTTCCAACGACACATACCGTGAC CTCCAAGGCGAGCGGCAGGAGTGGAAGCGCTTCATCGAGGAGCGGCTGCTCATGTACTCCTT CGTCAATGACAAGTTTATGCCCCCTGATGACCCACTGGGCCGGCACGGGCCCAGCCTGGACA ACTTCCTGCGTAAGAAGCCACTCACTTTGGAGCACAGGAAGCAGCCGTGTCCCTATGGAAGG AAATGCACCTATGGGATCAAGTGCCGATTCTTCCACCCAGAGCGGCCAAGCTGCCCCCAGCG CTCTGTGGCAGATGAGCTCCGTGCCAATGCTCTCCTCTCACCCCCCAGAGCCCCAAGCAAGG ACAAAAATGGCCGGCGGCCTTCACCTTCATCCCAGTCCAGCTCTCTGCTAACAGAGAGTGAG CAGTGCAGCCTGGATGGGAAGAAGCTGGGGGCCCAGGCATCCCCAGGGTCCCGCCAAGAGGG TCTAACACAGACCTATGCCCCATCAGGCAGGAGCCTCGCACCTAGCGGGGGCAGTGGCAGCA GCTTTGGGCCCACAGACTGGCTCCCACAGACGCTGGACTCACTCCCGTACGTCTCCCAGGAT TGCCTGGACTCGGGCATTGGCTCCCTGGAGAGCCAGATGTCGGAACTTTGGGGGGTTCGAGG AGGAGGCCCTGGTGAGCCGGGCCCACCCCGAGCCCCTTACACGGGCTACAGTCCCTATGGAT CTGAGCTCCCAGCCACCGCAGCCTTCTCTGCCTTTGGCCGGGCCATGGGTGCTGGCCACTTC AGTGTCCCTGCCGACTACCCACCCGCGCCCCCTGCCTTTCCACCTCGAGAGTACTGGTCTGA ACCATACCCACTGCCCCCACCCACATCAGTCCTTCAGGAGCCCCCAGTGCAGAGCCCAGGGG CTGGCAGGAGCCCGTGGGGCAGGGCAGACAGCCTGGCCAAGGAGCAGGCCAGCGTGTATACT AAGCTGTGTGGTGTGTTTCCCCCGCACCTGGTGGAGGCTGTGATGGGGCGCTTCCCACAGCT CCTGGACCCCCAGCAGCTGGCTGCCGAGATCCTCTCCTACAAGTCCCAGCACCCCAGTGAGT AA (SEQ ID NO: 650). An exemplary Regnase-1 gene sequence is provided at ENSEMBL Accession No. ENSG00000163874 (SEQ ID NO: 651).

By “Ring finger and CCCH-type zinc finger domains-containing 1(Roquin-1) polypeptide” is meant a protein having at least about 85% amino acid sequence identity to NCBI Reference Sequence: NP_001287779.1, which is provided below, or a fragment thereof having immunomodulatory activity.

>NP_001287779.1 roquin-1 isoform a
[Homo sapiens]
(SEQ ID NO: 652)
MPVQAPQWTDFLSCPICTQTFDETIRKPISLGCGHTVCKMCLNKL
HRKACPFDQTTINTDIELLPVNSALLQLVGAQVPEQQPITLCSGV
EDTKHYEEAKKCVEELALYLKPLSSARGVGLNSTTQSVLSRPMQR
KLVTLVHCQLVEEEGRIRAMRAARSLGERTVTELILQHQNPQQLS
SNLWAAVRARGCQFLGPAMQEEALKLVLLALEDGSALSRKVLVLF
VVQRLEPRFPQASKTSIGHVVQLLYRASCFKVTKRDEDSSLMQLK
EEFRTYEALRREHDSQIVQIAMEAGLRIAPDQWSSLLYGDQSHKS
HMQSIIDKLQTPASFAQSVQELTIALQRTGDPANLNRLRPHLELL
ANIDPSPDAPPPTWEQLENGLVAVRTVVHGLVDYIQNHSKKGADQ
QQPPQHSKYKTYMCRDMKQRGGCPRGASCTFAHSQEELEKFRKMN
KRLVPRRPLSASLGQLNEVGLPSAAILPDEGAVDLPSRKPPALPN
GIVSTGNTVTQLIPRGTDPSYDSSLKPGKIDHLSSSAPGSPPDLL
ESVPKSISALPVNPHSIPPRGPADLPPMPVTKPLQMVPRGSQLYP
AQQTDVYYQDPRGAAPPFEPAPYQQGMYYTPPPQCVSRFVRPPPS
APEPAPPYLDHYPPYLQERVVNSQYGTQPQQYPPIYPSHYDGRRV
YPAPSYTREEIFRESPIPIEIPPAAVPSYVPESRERYQQIESYYP
VAPHPTQIRPSYLREPPYSRLPPPPQPHPSLDELHRRRKEIMAQL
EERKVISPPPFAPSPTLPPTFHPEEFLDEDLKVAGKYKGNDYSQY
SPWSCDTIGSYIGTKDAKPKDVVAAGSVEMMNVESKGMRDQRLDL
QRRAAETSDDDLIPFGDRPTVSRFGAISRTSKTIYQGAGPMQAMA
PQGAPTKSINISDYSPYGTHGGWGASPYSPHQNIPSQGHFSERER
ISMSEVASHGKPLPSAEREQLRLELQQLNHQISQQTQLRGLEAVS
NRLVLQREANTLAGQSQPPPPPPPKWPGMISSEQLSLELHQVERE
IGKRTRELSMENQCSLDMKSKLNTSKQAENGQPEPQNKVPAEDLT
LTFSSDVPNGSALTQENISLLSNKTSSLNLSEDPEGGGDNNDSQR
SGVTPSSAP.

By “Ring finger and CCCH-type zinc finger domains-containing 1(Roquin-1) polynucleotide” is meant a polynucleotide encoding a Roquin-1 polypeptide, as well as the introns, exons, 3′ untranslated regions, 5′ untranslated regions, and regulatory sequences associated with its expression, or fragments thereof. In embodiments, a Roquin-1 polynucleotide is the genomic sequence, cDNA, mRNA, or gene associated with and/or required for Roquin-1 expression. An exemplary Roquin-1 nucleic acid sequence is provided below (NCBI Ref. Seq. Accession No. NM_001300850.1:88-3492).

>NM_001300850.1:88-3492 Homo sapiens Ring Finger and CCCH-Type Domains 1 (RC3H1), Transcript Variant 1, mRNA
ATGCCTGTACAAGCTCCACAATGGACGGATTTCCTCTCCTGCCCAATTTGCACTCAGACTTT CGACGAAACAATTCGAAAGCCCATCAGTTTGGGTTGTGGCCATACTGTCTGCAAGATGTGCC TGAATAAACTCCACCGCAAGGCTTGCCCATTTGACCAGACCACTATCAATACAGACATTGAG CTCCTCCCTGTGAACTCAGCATTGCTGCAGCTCGTGGGTGCTCAGGTCCCTGAGCAGCAGCC TATTACTTTGTGTAGTGGGGTTGAAGACACAAAGCATTATGAGGAAGCCAAGAAATGTGTAG AAGAATTAGCATTGTACTTAAAACCGCTCAGCAGTGCTAGAGGAGTGGGTCTGAACAGCACT ACTCAGAGTGTTCTGAGTCGCCCAATGCAGAGGAAGCTTGTGACTCTAGTCCACTGTCAACT AGTAGAAGAAGAAGGCAGGATTCGTGCCATGAGGGCAGCTCGATCTTTAGGTGAACGAACAG TTACAGAGCTCATTCTCCAGCACCAGAATCCTCAGCAACTCTCTTCCAATCTTTGGGCAGCA GTAAGGGCTAGGGGATGCCAGTTCCTTGGACCAGCAATGCAGGAGGAAGCTTTAAAGTTGGT TCTGCTGGCCTTAGAAGATGGTTCTGCTTTGTCAAGAAAAGTATTGGTTCTGTTTGTGGTGC AAAGATTGGAGCCACGGTTTCCTCAAGCCTCTAAAACTAGCATTGGGCATGTTGTTCAGCTC CTTTATAGAGCCTCCTGTTTCAAGGTCACCAAACGTGATGAAGACTCTTCTTTGATGCAGCT GAAAGAAGAATTTAGAACCTATGAAGCTCTGCGGCGAGAACATGACTCCCAGATAGTGCAGA TTGCTATGGAAGCAGGCTTACGAATTGCACCGGACCAGTGGTCCTCTTTGCTTTATGGAGAC CAGTCTCACAAATCTCATATGCAGTCCATTATTGACAAGTTGCAGACTCCAGCCTCTTTTGC ACAGAGTGTTCAGGAACTAACAATTGCTCTCCAGCGAACTGGAGACCCAGCAAACTTGAACC GACTAAGACCCCATTTGGAGCTGTTAGCAAACATTGACCCTAGTCCAGATGCTCCTCCTCCT ACTTGGGAACAGCTGGAAAATGGGCTGGTTGCTGTGCGTACAGTGGTACATGGGCTGGTTGA TTATATCCAGAACCACAGCAAAAAAGGAGCAGATCAGCAGCAGCCTCCACAGCATAGCAAAT ACAAAACATACATGTGTCGAGATATGAAGCAGAGAGGAGGATGCCCTCGTGGGGCCAGCTGT ACATTTGCACACTCACAGGAGGAACTGGAAAAATTTCGTAAAATGAATAAGCGCCTGGTTCC GAGAAGACCCTTGAGTGCCTCTTTGGGTCAACTTAATGAGGTGGGCCTGCCTTCAGCAGCTA TCCTTCCAGATGAAGGTGCAGTGGATCTCCCTAGCAGAAAACCTCCTGCTCTGCCAAATGGA ATTGTATCAACAGGGAATACAGTAACACAGCTTATTCCGCGAGGGACAGACCCCAGCTATGA TTCTAGTCTGAAACCAGGAAAAATAGATCATCTGAGCAGTAGTGCTCCTGGATCCCCTCCTG ACCTGCTAGAATCTGTTCCTAAGAGTATTTCTGCCTTACCTGTGAATCCACATTCTATACCT CCAAGGGGGCCAGCAGATCTGCCTCCAATGCCTGTTACCAAACCACTTCAGATGGTACCTCG AGGTTCTCAGTTATATCCAGCACAACAGACGGATGTTTATTATCAGGATCCTCGAGGAGCAG CTCCGCCATTTGAACCAGCACCTTATCAGCAGGGTATGTATTATACTCCACCACCACAATGT GTGTCCCGCTTTGTCCGACCTCCACCATCTGCTCCTGAACCTGCTCCTCCCTACTTGGATCA TTATCCACCCTACCTCCAAGAACGTGTTGTAAACTCTCAGTATGGCACACAGCCACAGCAGT ACCCACCTATATACCCATCTCACTATGATGGCCGTCGAGTGTACCCTGCTCCGTCTTACACA AGAGAAGAGATATTCCGAGAAAGCCCTATACCCATTGAGATTCCACCTGCAGCAGTACCATC GTATGTACCAGAATCCAGAGAAAGATACCAACAGATCGAGAGTTACTATCCAGTGGCTCCTC ATCCAACTCAGATCAGACCTTCGTACCTCAGAGAACCTCCTTATAGCCGGCTTCCTCCTCCT CCCCAGCCTCATCCTAGTCTAGATGAACTACATCGCCGACGAAAGGAAATAATGGCCCAGCT AGAGGAAAGAAAGGTTATCTCTCCACCTCCTTTTGCACCTTCACCAACCTTGCCTCCTACCT TTCATCCGGAAGAATTTTTGGATGAAGACTTGAAGGTAGCTGGGAAATACAAAGGAAATGAT TATAGCCAATACTCTCCCTGGTCATGTGACACCATCGGCTCCTACATTGGAACCAAAGATGC AAAACCCAAAGATGTTGTGGCAGCAGGGAGTGTGGAAATGATGAATGTGGAGAGTAAAGGAA TGAGGGACCAGCGATTAGATCTTCAGAGAAGAGCAGCAGAAACCAGTGATGATGACCTCATC CCATTTGGAGACCGACCAACAGTGTCTCGGTTTGGTGCCATCTCACGAACTTCCAAAACTAT ATATCAGGGTGCTGGTCCAATGCAGGCTATGGCACCTCAGGGAGCTCCTACAAAATCTATTA ACATTTCAGATTATAGTCCATATGGAACCCACGGTGGCTGGGGAGCTTCTCCATATTCACCT CATCAAAACATACCTTCTCAGGGACACTTCAGTGAGAGGGAGAGAATATCTATGTCAGAAGT GGCCAGTCATGGAAAACCCCTTCCATCTGCTGAGAGAGAACAGCTACGACTAGAATTGCAGC AATTGAACCATCAGATTAGCCAGCAGACCCAGCTACGTGGACTAGAGGCTGTTAGTAACAGG CTGGTGTTGCAGAGGGAGGCAAACACCCTGGCAGGCCAGTCACAGCCCCCACCACCGCCACC TCCAAAATGGCCTGGGATGATCTCAAGTGAGCAGTTGAGCTTGGAACTGCACCAGGTGGAAA GGGAAATCGGGAAGAGAACACGGGAACTGAGTATGGAAAACCAGTGTTCTCTGGACATGAAA AGCAAACTGAATACAAGTAAACAAGCAGAAAATGGACAACCAGAACCACAAAACAAGGTTCC GGCTGAGGACCTTACATTGACATTCAGCAGTGATGTACCAAATGGATCAGCCTTGACACAAG AGAATATCAGCCTCCTATCAAACAAGACCAGCTCTCTGAACCTGTCAGAGGACCCTGAGGGA GGAGGGGATAATAATGACTCCCAGAGATCAGGAGTTACTCCCAGTTCTGCTCCTTAA (SEQ ID NO: 653). An exemplary Roquin-1 gene sequence is provided at ENSEMBL Accession No. ENSG00000135870 (SEQ ID NO: 654).

By “Suppressor of cytokine signaling 1 (SOCS1) polypeptide” is meant a protein having at least about 85% amino acid sequence identity to GenBank Accession No. AAB62401.1, which is provided below, or a fragment thereof having immunomodulatory activity.

>AAB62401.1 suppressor of cytokine signalling-1
[Homo sapiens]
(SEQ ID NO: 655)
MVAHNQVAADNAVSTAAEPRRRPEPSSSSSSSPAAPARPRPCPAV
PAPAPGDTHFRTFRSHADYRRITRASALLDACGFYWGPLSVHGAH
ERLRAEPVGTFLVRDSRQRNCFFALSVKMASGPTSIRVHFQAGRF
HLDGSRESFDCLFELLEHYVAAPRRMLGAPLRQRRVRPLQELCRQ
RIVATVGRENLARIPLNPVLRDYLSSFPFQI.

By “Suppressor of cytokine signaling 1 (SOCS1) polynucleotide” is meant a polynucleotide encoding a SOCS1 polypeptide, as well as the introns, exons, 3′ untranslated regions, 5′ untranslated regions, and regulatory sequences associated with its expression, or fragments thereof. In embodiments, a SOCS1 polynucleotide is the genomic sequence, cDNA, mRNA, or gene associated with and/or required for SOCS1 expression. An exemplary SOCS1 nucleic acid sequence is provided below (GenBank Accession No. U88326.1:24-659).

>U88326.1:24-659 Human Suppressor of Cytokine Signalling-1 (SOCS-1) mRNA, Complete Cds
ATGGTAGCACACAACCAGGTGGCAGCCGACAATGCAGTCTCCACAGCAGCAGAGCCCCGACG GCGGCCAGAACCTTCCTCCTCTTCCTCCTCCTCGCCCGCGGCCCCCGCGCGCCCGCGGCCGT GCCCCGCGGTCCCGGCCCCGGCCCCCGGCGACACGCACTTCCGCACATTCCGTTCGCACGCC GATTACCGGCGCATCACGCGCGCCAGCGCGCTCCTGGACGCCTGCGGATTCTACTGGGGGCC CCTGAGCGTGCACGGGGCGCACGAGCGGCTGCGCGCCGAGCCCGTGGGCACCTTCCTGGTGC GCGACAGCCGCCAGCGGAACTGCTTTTTCGCCCTTAGCGTGAAGATGGCCTCGGGACCCACG AGCATCCGCGTGCACTTTCAGGCCGGCCGCTTTCACCTGGATGGCAGCCGCGAGAGCTTCGA CTGCCTCTTCGAGCTGCTGGAGCACTACGTGGCGGCGCCGCGCCGCATGCTGGGGGCCCCGC TGCGCCAGCGCCGCGTGCGGCCGCTGCAGGAGCTGTGCCGCCAGCGCATCGTGGCCACCGTG GGCCGCGAGAACCTGGCTCGCATCCCCCTCAACCCCGTCCTCCGCGACTACCTGAGCTCCTT CCCCTTCCAGATTTGA (SEQ ID NO: 656). An exemplary SOCS1 gene sequence is provided at ENSEMBL Accession No. ENSG00000185338 (SEQ ID NO: 657).

By “SRY-box 4 (SOX4) polypeptide” is meant a protein having at least about 85% amino acid sequence identity to GenBank Accession No. CAA50018.1, which is provided below, or a fragment thereof having immunomodulatory activity.

>CAA50018.1 SOX-4 protein
[Homo sapiens]
(SEQ ID NO: 658)
MVQQTNNAENTEALLAGESSDSGAGLELGIASSPTPGSTASTGGK
ADDPSWCKTPSGHIKRPMNAFMVWSQIERRKIMEQSPDMHNAEIS
KRLGKRWKLLKDSDKIPFIREAERLRLKHMADYPDYKYRPRKKVK
SGNANSSSSAAASSKPGEKGDKVGGSGGGGHGGGGGGGSSNAGGG
GGGASGGGANSKPAQKKSCGSKVAGGAGGGVSKPHAKLILAGGGG
GGKAAAAAAASFAAEQAGAAALLPLGAAADHHSLYKARTPSASAS
ASSAASASAALAAPGKHLAEKKVKRVYLFGGLGTSSSPVGGVGAG
ADPSDPLGLYEEEGAGCSPDAPSLSGRSSAASSPAAGRSPADHRG
YASLRAASPAPSSAPSHASSSASSHSSSSSSSGSSSSDDEFEDDL
LDLNPSSNFESMSLGSFSSSSALDRDLDFNFEPGSGSHFEFPDYC
TPEVSEMISGDWLESSISNLVFTY.

By “SRY-box 4 (SOX4) polynucleotide” is meant a polynucleotide encoding a SOX4 polypeptide, as well as the introns, exons, 3′ untranslated regions, 5′ untranslated regions, and regulatory sequences associated with its expression, or fragments thereof. In embodiments, a SOX4 polynucleotide is the genomic sequence, cDNA, mRNA, or gene associated with and/or required for SOX4 expression. An exemplary SOX4 nucleic acid sequence is provided below (GenBank Accession No. X70683.1:351-1775).

>X70683.1:351-1775 H. sapiens mRNA for SOX-4 Protein
ATGGTGCAGCAAACCAACAATGCCGAGAACACGGAAGCGCTGCTGGCCGGCGAGAGCTCGGA CTCGGGCGCCGGCCTCGAGCTGGGAATCGCCTCCTCCCCCACGCCCGGCTCCACCGCCTCCA CGGGCGGCAAGGCCGACGACCCGAGCTGGTGCAAGACCCCGAGTGGGCACATCAAGCGACCC ATGAACGCCTTCATGGTGTGGTCGCAGATCGAGCGGCGCAAGATCATGGAGCAGTCGCCCGA CATGCACAACGCCGAGATCTCCAAGCGGCTGGGCAAACGCTGGAAGCTGCTCAAAGACAGCG ACAAGATCCCTTTCATTCGAGAGGCGGAGCGGCTGCGCCTCAAGCACATGGCTGACTACCCC GACTACAAGTACCGGCCCAGGAAGAAGGTGAAGTCCGGCAACGCCAACTCCAGCTCCTCGGC CGCCGCCTCCTCCAAGCCGGGGGAGAAGGGAGACAAGGTCGGTGGCAGTGGCGGGGGCGGCC ATGGGGGCGGCGGCGGCGGCGGGAGCAGCAACGCGGGGGGAGGAGGCGGCGGTGCGAGTGGC GGCGGCGCCAACTCCAAACCGGCGCAGAAAAAGAGCTGCGGCTCCAAAGTGGCGGGCGGCGC GGGCGGTGGGGTTAGCAAACCGCACGCCAAGCTCATCCTGGCAGGCGGCGGCGGCGGCGGGA AAGCAGCGGCTGCCGCCGCCGCCTCCTTCGCCGCCGAACAGGCGGGGGCCGCCGCCCTGCTG CCCCTGGGCGCCGCCGCCGACCACCACTCGCTGTACAAGGCGCGGACTCCCAGCGCCTCGGC CTCCGCCTCCTCGGCAGCCTCGGCCTCCGCAGCGCTCGCGGCCCCGGGCAAGCACCTGGCGG AGAAGAAGGTGAAGCGCGTCTACCTGTTCGGCGGCCTGGGCACGTCGTCGTCGCCCGTGGGC GGCGTGGGCGCGGGAGCCGACCCCAGCGACCCCCTGGGCCTGTACGAGGAGGAGGGCGCGGG CTGCTCGCCCGACGCGCCCAGCCTGAGCGGCCGCAGCAGCGCCGCCTCGTCCCCCGCCGCCG GCCGCTCGCCCGCCGACCACCGCGGCTACGCCAGCCTGCGCGCCGCCTCGCCCGCCCCGTCC AGCGCGCCCTCGCACGCGTCCTCCTCGGCCTCGTCCCACTCCTCCTCTTCCTCCTCCTCGGG CTCCTCGTCCTCCGACGACGAGTTCGAAGACGACCTGCTCGACCTGAACCCCAGCTCAAACT TTGAGAGCATGTCCCTGGGCAGCTTCAGTTCGTCGTCGGCGCTCGACCGGGACCTGGATTTT AACTTCGAGCCCGGCTCCGGCTCGCACTTCGAGTTCCCGGACTACTGCACGCCCGAGGTGAG CGAGATGATCTCGGGAGACTGGCTCGAGTCCAGCATCTCCAACCTGGTTTTCACCTACTGA (SEQ ID NO: 659). An exemplary SOX4 gene sequence is provided at ENSEMBL Accession No. ENSG00000124766 (SEQ ID NO: 670).

By (“T-BOX transcription factor 21 (T-bet) polypeptide” is meant a protein having at least about 85% amino acid sequence identity to NCBI Reference Sequence: NP_037483.1, provided below, or a fragment thereof and having immunomodulatory activity.

>NP_037483.1 T-box transcription factor TBX21
[Homo sapiens]
(SEQ ID NO: 661)
MGIVEPGCGDMLTGTEPMPGSDEGRAPGADPQHRYFYPEPGAQDA
DERRGGGSLGSPYPGGALVPAPPSRFLGAYAYPPRPQAAGFPGAG
ESFPPPADAEGYQPGEGYAAPDPRAGLYPGPREDYALPAGLEVSG
KLRVALNNHLLWSKFNQHQTEMIITKQGRRMFPFLSFTVAGLEPT
SHYRMFVDVVLVDQHHWRYQSGKWVQCGKAEGSMPGNRLYVHPDS
PNTGAHWMRQEVSFGKLKLTNNKGASNNVTQMIVLQSLHKYQPRL
HIVEVNDGEPEAACNASNTHIFTFQETQFIAVTAYQNAEITQLKI
DNNPFAKGFRENFESMYTSVDTSIPSPPGPNCQFLGGDHYSPLLP
NQYPVPSRFYPDLPGQAKDVVPQAYWLGAPRDHSYEAEFRAVSMK
PAFLPSAPGPTMSYYRGQEVLAPGAGWPVAPQYPPKMGPASWFRP
MRTLPMEPGPGGSEGRGPEDQGPPLVWTEIAPIRPESSDSGLGEG
DSKRRRVSPYPSSGDSSSPAGAPSPFDKEAEGQFYNYFPN.

By “T-BOX transcription factor 21 (T-bet) polynucleotide” is meant a polynucleotide encoding a T-bet polypeptide, as well as the introns, exons, 3′ untranslated regions, 5′ untranslated regions, and regulatory sequences associated with its expression, or fragments thereof. In embodiments, a T-bet polynucleotide is the genomic sequence, cDNA, mRNA, or gene associated with and/or required for T-bet expression. An exemplary T-bet nucleic acid sequence is provided below (NCBI Ref. Seq. Accession No. NM_013351.2:220-1827).

>NM_013351.2:220-1827 Homo sapiens T-Box Transcription Factor 21 (TBX21), mRNA
ATGGGCATCGTGGAGCCGGGTTGCGGAGACATGCTGACGGGCACCGAGCCGATGCCGGGGAG CGACGAGGGCCGGGCGCCTGGCGCCGACCCGCAGCACCGCTACTTCTACCCGGAGCCGGGCG CGCAGGACGCGGACGAGCGTCGCGGGGGCGGCAGCCTGGGGTCTCCCTACCCGGGGGGCGCC TTGGTGCCCGCCCCGCCGAGCCGCTTCCTTGGAGCCTACGCCTACCCGCCGCGACCCCAGGC GGCCGGCTTCCCCGGCGCGGGCGAGTCCTTCCCGCCGCCCGCGGACGCCGAGGGCTACCAGC CGGGCGAGGGCTACGCCGCCCCGGACCCGCGCGCCGGGCTCTACCCGGGGCCGCGTGAGGAC TACGCGCTACCCGCGGGACTGGAGGTGTCGGGGAAACTGAGGGTCGCGCTCAACAACCACCT GTTGTGGTCCAAGTTTAATCAGCACCAGACAGAGATGATCATCACCAAGCAGGGACGGCGGA TGTTCCCATTCCTGTCATTTACTGTGGCCGGGCTGGAGCCCACCAGCCACTACAGGATGTTT GTGGACGTGGTCTTGGTGGACCAGCACCACTGGCGGTACCAGAGCGGCAAGTGGGTGCAGTG TGGAAAGGCCGAGGGCAGCATGCCAGGAAACCGCCTGTACGTCCACCCGGACTCCCCCAACA CAGGAGCGCACTGGATGCGCCAGGAAGTTTCATTTGGGAAACTAAAGCTCACAAACAACAAG GGGGCGTCCAACAATGTGACCCAGATGATTGTGCTCCAGTCCCTCCATAAGTACCAGCCCCG GCTGCATATCGTTGAGGTGAACGACGGAGAGCCAGAGGCAGCCTGCAACGCTTCCAACACGC ATATCTTTACTTTCCAAGAAACCCAGTTCATTGCCGTGACTGCCTACCAGAATGCCGAGATT ACTCAGCTGAAAATTGATAATAACCCCTTTGCCAAAGGATTCCGGGAGAACTTTGAGTCCAT GTACACATCTGTTGACACCAGCATCCCCTCCCCGCCTGGACCCAACTGTCAATTCCTTGGGG GAGATCACTACTCTCCTCTCCTACCCAACCAGTATCCTGTTCCCAGCCGCTTCTACCCCGAC CTTCCTGGCCAGGCGAAGGATGTGGTTCCCCAGGCTTACTGGCTGGGGGCCCCCCGGGACCA CAGCTATGAGGCTGAGTTTCGAGCAGTCAGCATGAAGCCTGCATTCTTGCCCTCTGCCCCTG GGCCCACCATGTCCTACTACCGAGGCCAGGAGGTCCTGGCACCTGGAGCTGGCTGGCCTGTG GCACCCCAGTACCCTCCCAAGATGGGCCCGGCCAGCTGGTTCCGCCCTATGCGGACTCTGCC CATGGAACCCGGCCCTGGAGGCTCAGAGGGACGGGGACCAGAGGACCAGGGTCCCCCCTTGG TGTGGACTGAGATTGCCCCCATCCGGCCGGAATCCAGTGATTCAGGACTGGGCGAAGGAGAC TCTAAGAGGAGGCGCGTGTCCCCCTATCCTTCCAGTGGTGACAGCTCCTCCCCTGCTGGGGC CCCTTCTCCTTTTGATAAGGAAGCTGAAGGACAGTTTTATAACTATTTTCCCAACTGA (SEQ ID NO: 662). An exemplary T-bet gene sequence is provided at ENSEMBL Accession No. ENSG00000073861 (SEQ ID NO: 663).

By “Transducin-like enhancer protein (TLE1) polypeptide” is meant a protein having at least about 85% amino acid sequence identity to GenBank Accession No. AAA61192.1, which is provided below, or a fragment thereof having immunomodulatory activity.

>AAA61192.1 transducin-like enhancer protein
[Homo sapiens]
(SEQ ID NO: 664)
MFPQSRHPTPHQAAGQPFKFTIPESLDRIKEEFQFLQAQYHSLKL
ECEKLASEKTEMQRHYVMYYEMSYGLNIEMHKQTEIAKRLNTICA
QVIPFLSQEHQQQVAQAVERAKQVTMAELNAIIGQQQLQAQHLSH
GHGPPVPLTPHPSGLQPPGIPPLGGSAGLLALSSALSGQSHLAIK
DDKKHHDAEHHRDREPGTSNSLLVPDSLRGTDKRRNGPEFSNDIK
KRKVDDKDSSHYDSDGDKSDDNLVVDVSNEDPSSPRASPAHSPRE
NGIDKNRLLKKDASSSPASTASSASSTSLKSKEMSLHEKASTPVL
KSSTPTPRSDMPTPGTSATPGLRPGLGKPPAIDPLVNQAAAGLRT
PLAVPGPYPAPFGMVPHAGMNGELTSPGAAYASLHNMSPQMSAAA
ARGRRGRYGRSPMVGFDPPPHMRVPTIPPNLAGIPGGKPAYSFHV
TADGQMQPVPFPPTPLIGPGIPRHARQINTLNHGEVVCAVTISNP
TRHVYTGGKGCVKVWDISHPGNKSPVSQLDCLNRDNYIRSCKLLP
DGCTLIVGGEASTLSIWDLAAPTPRIKAELTSSAPACYALAISPD
SKVCFSCCSDGNIAVWDLHNQTLVRQFQGHTDGASCIDISNDGTK
LWTGGLDNTVRSWDLREGRQLQQHDFTSQIFSLGYCPTGEWLAVG
MESSNVEVLHVNKPDKYQLHLHESCVLSLKFAYCGKWFVSTGKDN
LLNAWRTPYGASIFQSKESSSVLSCDISVDDKYIVTGSGDKKATV
YEVIY.

By “Transducin-like enhancer protein (TLE1) polynucleotide” is meant a polynucleotide encoding a TLE1 polypeptide, as well as the introns, exons, 3′ untranslated regions, 5′ untranslated regions, and regulatory sequences associated with its expression, or fragments thereof. In embodiments, a TLE1 polynucleotide is the genomic sequence, cDNA, mRNA, or gene associated with and/or required for TLE1 expression. An exemplary TLE1 nucleic acid sequence is provided below (GenBank Accession No. M99435.1:26-2338).

>M99435.1:26-2338 Human Transducin-Like Enhancer Protein (TLE1) mRNA, Complete Cds
ATGTTCCCGCAGAGCCGGCACCCGACGCCGCACCAGGCTGCAGGCCAGCCCTTCAAGTTCAC TATCCCGGAGTCCCTGGACCGGATTAAAGAGGAATTCCAGTTCCTGCAGGCGCAGTATCACA GCCTTAAATTGGAATGTGAGAAACTGGCAAGTGAAAAGACAGAAATGCAGAGGCACTATGTG ATGTATTATGAAATGTCATATGGATTAAACATTGAAATGCACAAACAGACTGAAATCGCCAA GAGATTGAATACGATTTGTGCACAAGTCATCCCATTTCTGTCTCAGGAACATCAACAACAGG TGGCCCAGGCTGTTGAACGTGCCAAACAGGTGACCATGGCAGAGTTGAATGCCATCATCGGG CAGCAGCAGTTGCAAGCTCAGCATCTTTCTCATGGCCACGGACCCCCAGTTCCCCTTACGCC TCACCCTTCGGGACTTCAGCCTCCTGGAATCCCGCCCCTCGGGGGCAGTGCCGGCCTTCTTG CGCTGTCTAGTGCTCTGAGTGGGCAGTCTCACTTGGCAATAAAAGATGACAAGAAGCACCAC GATGCAGAGCACCACAGAGACAGAGAGCCGGGCACAAGTAATTCCCTCCTGGTCCCAGACAG TCTAAGAGGCACAGATAAACGCAGAAATGGACCTGAATTTTCCAATGACATCAAGAAAAGGA AGGTGGATGATAAGGACTCCAGCCACTATGACAGTGATGGTGACAAAAGCGATGACAACTTA GTTGTGGATGTGTCTAATGAGGACCCTTCTTCTCCGCGAGCAAGCCCTGCCCACTCGCCCCG GGAAAATGGAATCGACAAAAATCGCCTGCTAAAGAAGGATGCTTCTAGCAGTCCAGCTTCCA CGGCCTCCTCGGCAAGTTCCACTTCTTTGAAATCCAAAGAAATGAGCTTGCATGAAAAAGCC AGCACGCCTGTTCTGAAATCCAGCACACCAACGCCTCGGAGCGACATGCCAACGCCGGGCAC CAGCGCCACTCCAGGCCTCCGTCCAGGTCTCGGCAAGCCTCCAGCCATAGACCCCCTCGTTA ACCAAGCGGCAGCTGGCTTGAGGACACCCCTGGCAGTGCCCGGCCCATATCCTGCTCCTTTT GGGATGGTCCCCCACGCTGGCATGAACGGCGAGCTGACCAGCCCAGGCGCTGCCTACGCCAG TTTACACAACATGTCGCCCCAGATGAGCGCCGCAGCCGCCCGCGGCCGCCGTGGTCGGTACG GGCGCTCCCCCATGGTGGGGTTTGATCCTCCCCCTCACATGAGAGTACCTACCATTCCTCCA AACCTGGCAGGAATCCCTGGGGGGAAACCTGCATACTCCTTCCACGTTACTGCAGACGGTCA GATGCAGCCTGTCCCTTTTCCCCCGACGCCCCTCATCGGACCCGGAATCCCCCGGCATGCTC GCCAGATCAACACCCTCAACCACGGGGAGGTGGTGTGCGCTGTGACCATCAGCAACCCCACG AGACACGTGTACACAGGCGGGAAGGGCTGCGTCAAGGTCTGGGACATCAGCCACCCTGGCAA TAAGAGCCCTGTCTCCCAGCTCGACTGTCTGAACAGAGACAATTATATCCGTTCCTGTAAAT TGCTACCCGATGGCTGCACTCTCATAGTGGGAGGGGAAGCCAGTACTTTGTCCATTTGGGAC CTGGCGGCTCCAACCCCGCGCATCAAGGCGGAGCTGACGTCCTCGGCCCCCGCCTGCTACGC CCTGGCCATCAGCCCCGATTCCAAGGTCTGCTTCTCATGCTGCAGCGACGGCAACATCGCTG TGTGGGATCTGCACAACCAGACACTAGTGAGGCAATTCCAGGGCCACACAGACGGAGCCAGC TGTATTGACATTTCTAATGATGGCACCAAGCTCTGGACGGGTGGTTTGGACAACACAGTCAG GTCCTGGGACCTGCGCGAGGGGCGGCAGCTGCAGCAGCACGACTTCACCTCCCAGATCTTCT CCCTGGGGTACTGCCCCACCGGGGAGTGGCTGGCAGTGGGCATGGAGAGCAGCAATGTGGAG GTGCTGCACGTGAACAAGCCTGACAAGTACCAGCTGCACCTGCATGAGAGCTGCGTGCTGTC CCTGAAATTTGCTTACTGTGGTAAATGGTTTGTGAGTACTGGAAAAGATAACCTCCTCAATG CTTGGCGGACCCCCTATGGAGCCAGCATATTCCAGTCCAAAGAGTCCTCGTCAGTGCTTAGC TGTGACATCTCTGTGGATGATAAGTACATAGTCACTGGCTCGGGGGACAAGAAGGCTACAGT CTATGAAGTCATCTACTGA (SEQ ID NO: 665). An exemplary TLE1 gene sequence is provided at ENSEMBL Accession No. ENSG00000150990 (SEQ ID NO: 666).

By “Transmembrane protein 184B (TMEM184B) polypeptide” is meant a protein having at least about 85% amino acid sequence identity to NCBI Reference Sequence No.: NP_001182000.1, which is provided below, or a fragment thereof having immunomodulatory activity.

>NP_001182000.1 transmembrane protein 184B
isoform a [Homo sapiens]
(SEQ ID NO: 667)
MTVRGDVLAPDPASPTTAAASPSVSVIPEGSPTAMEQPVFLMTTA
AQAISGFFVWTALLITCHQIYMHLRCYSCPNEQRYIVRILFIVPI
YAFDSWLSLLFFTNDQYYVYFGTVRDCYEALVIYNFLSLCYEYLG
GESSIMSEIRGKPIESSCMYGTCCLWGKTYSIGFLRFCKQATLQF
CVVKPLMAVSTVVLQAFGKYRDGDFDVTSGYLYVTIIYNISVSLA
LYALFLFYFATRELLSPYSPVLKFFMVKSVIFLSFWQGMLLAILE
KCGAIPKIHSARVSVGEGTVAAGYQDFIICVEMFFAALALRHAFT
YKVYADKRLDAQGRCAPMKSISSSLKETMNPHDIVQDAIHNFSPA
YQQYTQQSTLEPGPTWRGGAHGLSRSHSLSGARDNEKTLLLSSDD
EF.

By “Transmembrane protein 184B (TMEM184B) polynucleotide” is meant a polynucleotide encoding a TMEM184B polypeptide, as well as the introns, exons, 3′ untranslated regions, 5′ untranslated regions, and regulatory sequences associated with its expression, or fragments thereof. In embodiments, a TMEM184B polynucleotide is the genomic sequence, cDNA, mRNA, or gene associated with and/or required for TMEM184B expression. An exemplary TMEM184B nucleic acid sequence is provided below (NCBI Ref. Seq. Accession No. NM_001195071.1:225-1448).

>NM_001195071.1:225-1448 Homo sapiens Transmembrane Protein 184B (TMEM184B), Transcript Variant 2, mRNA
ATGACAGTGAGGGGGGATGTGCTGGCCCCGGATCCAGCGTCGCCCACGACCGCAGCAGCCTC GCCCAGCGTCTCCGTGATCCCCGAGGGCAGCCCCACTGCCATGGAGCAGCCTGTGTTCCTGA TGACAACTGCCGCTCAGGCCATCTCTGGCTTCTTCGTGTGGACGGCCCTGCTCATCACATGC CACCAGATCTACATGCACCTGCGCTGCTACAGCTGCCCCAACGAGCAGCGCTACATCGTGCG CATCCTCTTCATCGTGCCCATCTACGCCTTTGACTCCTGGCTCAGCCTCCTCTTCTTCACCA ACGACCAGTACTACGTGTACTTCGGCACCGTCCGCGACTGCTATGAGGCCTTGGTCATCTAT AATTTCCTGAGCCTGTGCTATGAGTACCTAGGAGGAGAAAGTTCCATCATGTCGGAGATCAG AGGAAAACCCATTGAGTCCAGCTGTATGTATGGCACCTGCTGCCTCTGGGGAAAGACTTATT CCATCGGATTTCTGAGGTTCTGCAAACAGGCCACCCTGCAGTTCTGTGTGGTGAAGCCACTC ATGGCGGTCAGCACTGTGGTCCTCCAGGCCTTCGGCAAGTACCGGGATGGGGACTTTGACGT CACCAGTGGCTACCTCTACGTGACCATCATCTACAACATCTCCGTCAGCCTGGCCCTCTACG CCCTCTTCCTCTTCTACTTCGCCACCCGGGAGCTGCTCAGCCCCTACAGCCCCGTCCTCAAG TTCTTCATGGTCAAGTCCGTCATCTTTCTTTCCTTCTGGCAAGGCATGCTCCTGGCCATCCT GGAGAAGTGTGGGGCCATCCCCAAAATCCACTCGGCCCGCGTGTCGGTGGGCGAGGGCACCG TGGCTGCCGGCTACCAGGACTTCATCATCTGTGTGGAGATGTTCTTTGCAGCCCTGGCCCTG CGGCACGCCTTCACCTACAAGGTCTATGCTGACAAGAGGCTGGACGCACAAGGCCGCTGTGC CCCCATGAAGAGCATCTCCAGCAGCCTCAAGGAGACCATGAACCCGCACGACATCGTGCAGG ACGCCATCCACAACTTCTCACCTGCCTACCAGCAGTACACGCAGCAGTCCACCCTGGAGCCT GGGCCCACCTGGCGTGGTGGCGCCCACGGCCTCTCCCGCTCCCACAGCCTCAGTGGCGCCCG CGACAACGAGAAGACTCTCCTGCTCAGCTCTGATGATGAATTCTAG (SEQ ID NO: 668). An exemplary TMEM184B gene sequence is provided at ENSEMBL Accession No. ENSG00000198792 (SEQ ID NO: 669).

By “Transmembrane protein 222 (TMEM222) polypeptide” is meant a protein having at least about 85% amino acid sequence identity to GenBank Accession No. AAH90039.1, which is provided below, or a fragment thereof having immunomodulatory activity.

>AAH90039.1 TMEM222 protein [Homo sapiens]
(SEQ ID NO: 670)
MAFGKPAKYWKLDPAQVYASGPNAWDTAVHDASEEYKHRMHNLCCDNCH
SHVALALNLMRYNNSTNWNMVTLCFFCLLYGKYVSVGAFVKTWLPFILL
LGIILTVSLVFNLR.

By “Transmembrane protein 222 (TMEM222) polynucleotide” is meant a polynucleotide encoding a TMEM222 polypeptide, as well as the introns, exons, 3′ untranslated regions, 5′ untranslated regions, and regulatory sequences associated with its expression, or fragments thereof. In embodiments, a TMEM222 polynucleotide is the genomic sequence, cDNA, mRNA, or gene associated with and/or required for TMEM222 expression. An exemplary TMEM222 nucleic acid sequence is provided below (GenBank Accession No. BC090039.1:387-725).

>BC090039.1:387-725 Homo sapiens Transmembrane Protein 222, mRNA (cDNA Clone MGC:111002 IMAGE:6181849), Complete Cds
ATGGCCTTTGGAAAGCCTGCCAAGTACTGGAAGTTGGACCCTGCTCAGGTCTATGCTAGCGG GCCCAACGCATGGGACACGGCTGTGCACGACGCCTCTGAGGAGTACAAGCACCGCATGCACA ATCTCTGCTGTGACAACTGCCACTCGCACGTGGCATTGGCCCTGAATCTGATGCGCTACAAC AACAGCACCAACTGGAATATGGTGACGCTCTGCTTCTTCTGCCTGCTCTACGGGAAGTACGT CAGCGTTGGGGCCTTCGTGAAGACCTGGCTGCCCTTCATCCTTCTCCTGGGCATCATCCTCA CCGTCAGCCTGGTCTTTAACCTCCGGTGA (SEQ ID NO: 671). An exemplary TMEM222 gene sequence is provided at ENSEMBL Accession No. ENSG00000186501 (SEQ ID NO: 672).

By “Tumor necrosis factor alpha (TNFα or TNFa) polypeptide” is meant a protein having at least about 85% amino acid sequence identity to GenBank Accession No. CAA26669.1, provided below, or a fragment thereof and having immunomodulatory activity.

>CAA26669.1 TNF-alpha [Homo sapiens]
(SEQ ID NO: 673)
MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL.

By “Tumor necrosis factor alpha (TNFα or TNFa) polynucleotide” is meant a polynucleotide encoding an Tumor Necrosis Factor Alpha polypeptide, as well as the introns, exons, 3′ untranslated regions, 5′ untranslated regions, and regulatory sequences associated with its expression, or fragments thereof. In embodiments, a Tumor Necrosis Factor Alpha polynucleotide is the genomic sequence, cDNA, mRNA, or gene associated with and/or required for Tumor Necrosis Factor Alpha expression. An exemplary Tumor Necrosis Factor Alpha nucleic acid sequence is provided below (GenBank Accession No. X02910.1: 796-981,1589-1634,1822-1869,2171-2592).

### >X02910.1:796-981,1589-1634,1822-1869,2171-2592 Human Gene for Tumor Necrosis Factor (TNF-Alpha)

ATGAGCACTGAAAGCATGATCCGGGACGTGGAGCTGGCCGAGGAGGCGCTCCCCAAGAAGAC AGGGGGGCCCCAGGGCTCCAGGCGGTGCTTGTTCCTCAGCCTCTTCTCCTTCCTGATCGTGG CAGGCGCCACCACGCTCTTCTGCCTGCTGCACTTTGGAGTGATCGGCCCCCAGAGGGAAGAG TTCCCCAGGGACCTCTCTCTAATCAGCCCTCTGGCCCAGGCAGTCAGATCATCTTCTCGAAC CCCGAGTGACAAGCCTGTAGCCCATGTTGTAGCAAACCCTCAAGCTGAGGGGCAGCTCCAGT GGCTGAACCGCCGGGCCAATGCCCTCCTGGCCAATGGCGTGGAGCTGAGAGATAACCAGCTG GTGGTGCCATCAGAGGGCCTGTACCTCATCTACTCCCAGGTCCTCTTCAAGGGCCAAGGCTG CCCCTCCACCCATGTGCTCCTCACCCACACCATCAGCCGCATCGCCGTCTCCTACCAGACCA AGGTCAACCTCCTCTCTGCCATCAAGAGCCCCTGCCAGAGGGAGACCCCAGAGGGGGCTGAG GCCAAGCCCTGGTATGAGCCCATCTATCTGGGAGGGGTCTTCCAGCTGGAGAAGGGTGACCG ACTCAGCGCTGAGATCAATCGGCCCGACTATCTCGACTTTGCCGAGTCTGGGCAGGTCTACT TTGGGATCATTGCCCTGTGA (SEQ ID NO: 674). An exemplary TNFα gene sequence is provided at ENSEMBL Accession No. ENSG00000232810 (SEQ ID NO: 675).

By “T cell receptor alpha chain (TRAC) polypeptide” is meant a protein having at least about 85% amino acid sequence identity to GenBank Accession No.: AAO72258.1, which is provided below, or a fragment thereof having immunomodulatory activity.

>AAO72258.1 T cell receptor alpha chain
[Homo sapiens]
(SEQ ID NO: 676)
METLLGVSLVILWLQLARVNSQQGEEDPQALSIQEGENATMNCSYKTSI
NNLQWYRQNSGRGLVHLILIRSNEREKHSGRLRVTLDTSKKSSSLLITA
SRAADTASYFCATANAGGTSYGKLTFGQGTILTVHPNIQNPDPAVYQLR
DSKSSDKSVCLFTDFDSQTNVSQSKDSDVYITDKTVLDMRSMDFKSNSA
VAWSNKSDFACANAFNNSIIPEDTFFPSPESSCDVKLVEKSFETDTNLN
FQNLSVIGFRILLLKVAGFNLLMTLRLWSS.

By “T cell receptor alpha chain (TRAC) polynucleotide” is meant a polynucleotide encoding a TRAC polypeptide, as well as the introns, exons, 3′ untranslated regions, 5′ untranslated regions, and regulatory sequences associated with its expression, or fragments thereof. In embodiments, a TRAC polynucleotide is the genomic sequence, cDNA, mRNA, or gene associated with and/or required for TRAC expression. An exemplary TRAC nucleic acid sequence is provided below (GenBank Accession No. AY247834.1).

>AY247834.1 Homo sapiens T Cell Receptor Alpha Chain (TCRA) mRNA, TCRA-AV17S1 J52 AC Allele, Complete Cds
ATGGAAACTCTCCTGGGAGTGTCTTTGGTGATTCTATGGCTTCAACTGGCTAGGGTGAACAG TCAACAGGGAGAAGAGGATCCTCAGGCCTTGAGCATCCAGGAGGGTGAAAATGCCACCATGA ACTGCAGTTACAAAACTAGTATAAACAATTTACAGTGGTATAGACAAAATTCAGGTAGAGGC CTTGTCCACCTAATTTTAATACGTTCAAATGAAAGAGAGAAACACAGTGGAAGATTAAGAGT CACGCTTGACACTTCCAAGAAAAGCAGTTCCTTGTTGATCACGGCTTCCCGGGCAGCAGACA CTGCTTCTTACTTCTGTGCTACGGCTAATGCTGGTGGTACTAGCTATGGAAAGCTGACATTT GGACAAGGGACCATCTTGACTGTCCATCCAAATATCCAGAACCCTGACCCTGCCGTGTACCA GCTGAGAGACTCTAAATCCAGTGACAAGTCTGTCTGCCTATTCACCGATTTTGATTCTCAAA CAAATGTGTCACAAAGTAAGGATTCTGATGTGTATATCACAGACAAAACTGTGCTAGACATG AGGTCTATGGACTTCAAGAGCAACAGTGCTGTGGCCTGGAGCAACAAATCTGACTTTGCATG TGCAAACGCCTTCAACAACAGCATTATTCCAGAAGACACCTTCTTCCCCAGCCCAGAAAGTT CCTGTGATGTCAAGCTGGTCGAGAAAAGCTTTGAAACAGATACGAACCTAAACTTTCAAAAC CTGTCAGTGATTGGGTTCCGAATCCTCCTCCTGAAAGTGGCCGGGTTTAATCTGCTCATGAC GCTGCGGCTGTGGTCCAGCTGA (SEQ ID NO: 677). An exemplary TRAC gene sequence is provided at ENSEMBL Accession No. ENSG00000277734 (SEQ ID NO: 678).

By “adenine” or “9H-Purin-6-amine” is meant a purine nucleobase with the molecular formula C₅H₅N₅, having the structure

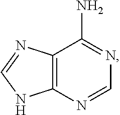

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to

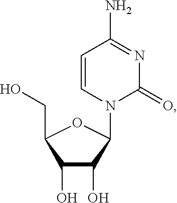

a ribose sugar via a glycosidic bond, having the structure and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any cell, small molecule chemical compound, antibody, nucleic acid molecule, or polypeptide, or fragments thereof.

“Allogeneic,” as used herein, refers to cells that are genetically dissimilar from the cells of a subject to which they are to be administered. In embodiments, allogeneic cells are administered to a genetically dissimilar and/or immunologically incompatible subject.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen. The term “antibody” includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)2, Fab, Fv, rlgG, and scFv fragments. Further non-limiting examples of antibodies include nanobodies and VHH domains. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody. Examples of these antibody fragments are described herein. Typically, an antibody binds to an antigen using a combination of a variable light chain (VL) and a corresponding variable heavy chain (VH) domain.

The term “antigen-binding domain” refers to a polypeptide or fragment thereof that binds an antigen. In embodiments, two or more antigen-binding domains can function in combination to bind an antigen. In some embodiments, the antigen-binding domain is a region of an antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n(D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C.

By “base editing efficiency” is meant the total percent of one or more target bases in a sample that have been modified using a base editor. In some cases, the base editing efficiency is calculated as the total percent of target polynucleotides in a sample containing a modified target base. In some instances, the base editing efficiency is calculated as the total percent of target polynucleotides in a sample containing a modification to one or more (e.g., 2, 3, 4, 5, 6, 7, 8, 9 or 10) of 2, 3, 4, 5, 6, 7, 8, 9, or 10 target bases. Methods for measuring base editing efficiency for a base editor are known in the art (see, e.g., Gaudelli, et al. Nature 551:464-471 (2017), the disclosure of which is incorporated herein in its entirety for all purposes). In some cases a base editing efficiency is a median base editing efficiency calculated across 2, 3, 4, 5, 6, 7, 8, 9, 10, 15, 20, 25, 30, 35, 40, 45, 50, 60, 70, 80, 90, 100 or more target sites.

By “base editing window” for a base editor is meant bases within a target polynucleotide sequence that can be modified using the base editor. In some embodiments, the position of the nucleobases in the target polynucleotide sequence are numbered relative to a protospacer adjacent motif (PAM) for which a nucleic acid programmable DNA binding protein (napDNAbp) domain of the base editor has specificity, where base 1 corresponds to the base immediately adjacent to the PAM. In some embodiments, the position of the nucleobases in the target polynucleotide sequence are numbered relative to the 5′ or 3′ end of a spacer of a guide polynucleotide used to guide a nucleic acid programmable DNA binding protein (napDNAbp) domain of the base editor to a target site, where base 1 corresponds to the 5′ or 3′ terminal base of the spacer.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor” or “CAR” is meant a synthetic or engineered receptor comprising an extracellular antigen binding domain operationally joined to one or more intracellular signaling domains where the CAR confers specificity for an antigen bound by the extracellular antigen binding domain onto an immune effector cell. In some cases, the intracellular signaling domain is a T cell signaling domain. In embodiments, the immune effector cell is a T cell, an NK cell, or a macrophage. In embodiments, the CAR is a SUPRA CAR, an anti-tag CAR, a TCR-CAR, or a TCR-like CAR (see, e.g., Guedan, et al. “Engineering and Design of Chimeric Antigen Receptors,” Methods and Clinical Development, 12:145-156 (2019); Poorebrahim, et al., “TCR-like CARs and TCR-CARs targeting neoepitopes: an emerging potential,” Cancer Gene Therapy, 28:581-589 (2021); and Minutolo, et al. “The Emergence of Universal Immune Receptor T Cell Therapy for Cancer,” Front Oncol., 9:176 (2019), the disclosures of which are incorporated herein by reference in their entireties for all purposes). In various embodiments, a CAR polypeptide of the disclosure comprises a polypeptide or polynucleotide sequence having about or at least about 85%, 90%, 95%, 96%, 97%, 98%, 99%, or more sequence identity to one or more polypeptide or polynucleotide sequence listed in Table 12 or Table 13 or to one or more of SEQ ID NOs: 830-833, or functional fragments thereof.

By “chimeric antigen receptor (CAR) T cell” or “CAR-T cell” is meant a T cell expressing a CAR that has antigen specificity determined by the antibody-derived targeting domain of the CAR. As used herein, “CAR-T cells” include T cells, regulatory T cells (T_{REG}), macrophages, or NK cells. As used herein, the term “CAR-T cells” includes cells engineered to express a CAR or a T cell receptor (TCR, sometimes referred to as TCR-CARs or TCR-like CARs). Methods of making CARs (e.g., for treatment of cancer) are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; Poorebrahim, et al., Cancer Gene Ther 28, 581-589 (2021), doi.org/10.1038/s41417-021-00307-7, PCT Pubs. WO2012/079000, WO2013/059593; and U.S. Pub. 2012/0213783, the disclosure of each of which is incorporated herein by reference herein in its entirety).

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe. In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with

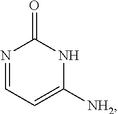

the molecular formula C₄H₅N₃O, having the structure NH₂ and corresponding to CAS No. 71-30-7.

By “cytidine” is meant a cytosine molecule attached to a ribose sugar via a glycosidic

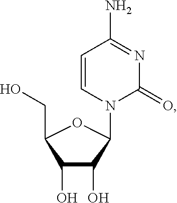

bond, having the structure, and corresponding to CAS No. 65-46-3. Its molecular formula is C₉H₁₃N₃O₅.

By “Cytidine Base Editor (CBE)” is meant a base editor comprising a cytidine deaminase.

By “Cytidine Base Editor (CBE) polynucleotide” is meant a polynucleotide encoding a CBE.

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, and PCT/US2016/058344. By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

By “cytotoxic capacity” is meant the ability of an immune effector cell to mediate the killing of a target cell. In some embodiments, the immune effector cell is a chimeric antigen receptor (CAR) expressing T cell (CAR-T cell). In some embodiments the target cell is a neoplastic cell. In various embodiments, cytotoxic capacity may be measured as killing of target cells, expression of activation markers (e.g., OX40, CD25), expression of stimulatory signals (e.g., ICOS, CD28), and/or expression of cytokines (e.g., IL-2, IL-6, IFN-gamma, TNF-alpha, granzyme B).

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In some embodiments, the disease is a cancer (e.g., a hematological cancer or a solid tumor). In some instances, the disease is a disease that can be treated using the base edited CAR-T cells of the disclosure. In one embodiment, the disease is a neoplasia or cancer. In some embodiments, the disease is a hematological cancer. By “hematological cancer” is meant a malignancy of immune system cells. In some embodiments, the hematological cancer is leukemia, myeloma, and/or lymphoma. Lymphomas and Leukemias are examples of “liquid cancers” or cancers present in the blood and are derived from the transformation of either a hematopoietic precursor in the bone marrow or a mature hematopoietic cell in the blood. Leukemias can be lymphoid or myeloid, and acute or chronic. In the case of myelomas, the transformed cell is a fully differentiated plasma cell that may be present as a dispersed collection of malignant cells or as a solid mass in the bone marrow. In the case of lymphomas, a transformed lymphocyte in a secondary lymphoid tissue generates a solid mass. Lymphomas are classified either Hodgkin lymphoma (HL) or non-Hodgkin lymphoma (NHL). In some instances, the hematologic cancer is a mantle cell lymphoma (MCL) or a B cell lymphoma (BCL). The disease in some cases is an autoimmune disease.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity.

By “effective amount” is meant the amount of an agent (e.g., a base editor, cell, such as a CAR-T cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “exhaustion” or “exhausted phenotype” is meant a condition in which an immune effector cell exhibits a reduction in capacity to effect an immune response. In embodiments, an exhausted immune cells shows a reduction in cytotoxicity, antitumor properties, proliferation, and/or cytokine production relative to a reference cell, such as a resting immune effector cell (e.g., a CAR-T cell not repeated or continuously exposed to an antigen). In some cases, exhaustion is associated with upregulation of repressor genes, increase in inhibitory receptors, reduction in quantity of immune effector cells, and/or reduced survival relative to a reference cell, such as a resting immune effector cell (e.g., a CAR-T cell not repeated or continuously exposed to an antigen). In some instances, the immune response is the killing of a cell expressing a target antigen. An immune effector cell can develop an exhausted phenotype when stimulated by a target antigen multiple times or continuously (see, e.g., FIG. 3A). In some cases, an exhausted immune effector cell shows reduced production of a cytokine selected from GZMB; IFNg; IL-2; and TNFa relative to a reference cell. In some instances, an exhausted immune effector cell shows reduced levels of activation relative to a reference cell, as indicated by reduced levels of CD25 expression. In some cases, an exhausted immune cell shows increased levels of EOMES and/or reduced levels of T-bet relative to a reference cell, or a reduction in Ki67 levels relative to a reference cell. The exhausted phenotype is epigenetically reinforced. Immune effector cell exhaustion is described further in Jiang, et al., “T-cell exhaustion in the tumor microenvironment,” Cell Death and Disease, 6:e1792 (2015), the disclosure of which is incorporated herein in its entirety by reference for all purposes.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immune cell” is meant a cell of the immune system capable of generating an immune response. Exemplary immune cells include, but are not limited to, T cells, NK cells, B cells, macrophages, hematopoietic stem cells, or precursors thereof. In embodiments, an immune cell is allogeneic to a subject to whom the cell is to be administered. In embodiments, an immune cell is from a donor and is allogeneic to a subject to which the immune cell will be administered after being modified according to the methods provided herein. The disclosure features methods for preparing modified allogeneic immune cells with improved characteristics (e.g., increased persistence in a subject) as well as the cells produced by these methods.

By “immune effector cell” is meant a lymphocyte, once activated, capable of effecting an immune response upon a target cell. In some embodiments, immune effector cells are effector T cells. In some embodiments, the effector T cell is a naïve CD8⁺ T cell, a cytotoxic T cell, a natural killer T (NKT) cell, a macrophage, a natural killer (NK) cell, or a regulatory T (Treg) cell. In some embodiments, immune effector cells are effector NK cells. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell).

By “immunomodulatory activity” is meant increasing, decreasing, or sustaining an immune response.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any agent or clinical parameter having an alteration that is associated with a disease or disorder. In embodiments, the agent is a polypeptide or polynucleotide and the alteration is in expression, level, structure, or activity. In embodiments, the disease or disorder is a neoplasia, such as a hematologic cancer (e.g., a lymphoma). A non-limiting examples of a markers include CD5, CD7, CD19, CD20, CD22, CD79B, and ROR1.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

“Neoplasia” refers to cells or tissues exhibiting abnormal growth or proliferation. The term neoplasia encompasses cancer, liquid, and solid tumors. In some embodiments, the neoplasia is a solid tumor. In other embodiments, the neoplasia is a liquid tumor. In some embodiments, the neoplasia is a hematological cancer. In some embodiments, the hematological cancer is leukemia, myeloma, and/or lymphoma. In some embodiments, the hematological cancer is a B cell cancer (e.g., a B cell lymphoma). In some embodiments, the B cell cancer is a lymphoma or a leukemia. In some cases, the leukemia comprises a pre-leukemia. In some instances, the neoplasia is a mantle cell lymphoma. In some cases, the leukemia is an acute leukemia. Acute leukemias include, for example, an acute myeloid leukemia (AML). Acute leukemias also include, for example, an acute lymphoid leukemia or an acute lymphocytic leukemia (ALL); ALL includes B-lineage ALL; T-lineage ALL; and T-cell acute lymphocytic leukemia (T-ALL).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSEFESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2cl, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2cl, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

By “persistence” in the context of an allogeneic transplant is meant the continued survival of a donor cell in a host organism. In some embodiments, allogeneic cell(s) comprising one or more of the edits described herein (e.g., a base edit in a CD5, CD3e, CD3g, B2M, and/or CHTA gene, or regulatory element(s) thereof; or knockdown of a CD5, TCRαβ, B2M, and/or CIITA polypeptide) persist in a subject allogeneic to the cells at higher levels over time post-infusion than corresponding unedited allogeneic control cells. In embodiments, the percentage of edited cells (e.g., T cells, NK cells, or lymphocytes) persisting in a subject at a given time point (e.g., 7 days, 14 days, 1 month, 3 months, 6 months, 9 months, or greater than 1, 2, or 3 years is at least about 10%, 15%, 20%, 25%, 30%, 35%, 40%, 45%, 50%, 60%, 70%, 80%, 90%, 95%, 99%, or 100% greater than the level of unedited control cells at the same time point. A cell(s) modified by methods of the present disclosure are more persistent than a reference unmodified cell(s).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide, which comprises protein domains from at least two different proteins.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 5%, 10%, 15%, 25%, 30%, 35%, 40%, 45% 50%, 55%, 60%, 65%, 70%, 75%, 80%, 85%, 90%, 95%, or 100%.

By “reference” is meant a standard or control condition. In one embodiment, the reference is a wild-type cell not base edited according to the methods provided herein or healthy cell. In other embodiments and without limitation, a reference is an untreated cell that is not subjected to a test condition, or is subjected to placebo or normal saline, medium, buffer, a control polynucleotide that does not encode a polypeptide of interest, and/or a control vector that does not harbor a polynucleotide of interest. In some cases, a reference is a healthy subject, such as a subject not having a neoplasia. In some embodiments, the reference is a cell lacking a nucleobase alteration and/or having an additional nucleobase alteration. The reference may be a cell that does not express one or more of the polypeptides described herein. The reference may be a subject before administration of a composition provided herein or treated according to a method provided herein and/or the subject before a change in a treatment (e.g., an alteration in dose or agent administered to the subject). A reference may be an antigen-naïve immune effector cell that has not previously or recently (e.g., within 1 hour, 2 hours, 3 hours, 4 hours, 5 hours, 6 hours, 12 hours, 1 day, 2 days, 3 days, 4 days, 5 days, 6 days, 7 days, one month, or longer) been stimulated by an antigen, that has not been continuously stimulated by an antigen, and/or that has not been repeatedly stimulated by an antigen (e.g., 1, 2, 3, 4, 5, 6, 7, 8, 9, or 10 times according to the method shown in FIG. 3A, or to a modified version of the method).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g. a sequence with at least about 85% sequence identity to a Cas9, such as Nme2Cas9 or spCas9).

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (VH) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In some embodiments, the split polypeptide is a nucleic acid programmable DNA binding protein (e.g., a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA glycosylase
inhibitor
(SEQ ID NO: 231)
MTNLSDIIEKETGKQLVIQESILMLPEEVEEVIGNKPESDILVHTAYDE
STDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a schematic diagram showing a summary of non-limiting embodiments of improved characteristics of immune effector cells (e.g., chimeric antigen receptor expressing T cells (CAR-T cells)) prepared according to the methods provided herein, referenced in FIG. 1 as “Next-gen CAR-T cells,” relative to immune effector cells prepared according to alternative methods.

FIG. 2 provides a schematic diagram showing how persistent antigen stimulation can lead to chimeric antigen receptor (CAR) T cell dysfunction (e.g., reduced cytotoxicity, proliferation, and/or cytokine production; and/or increased expression of repressor genes and/or inhibitory receptors; and/or reduced survival) in the form of an exhausted phenotype. CAR-T cell dysfunction may be characterized by exhaustion of antitumor properties, rapid contraction in quantity, and/or restricted survival.

FIGS. 3A to 3C provide a schematic diagram, a plot, and a bar graph showing an in vitro model of repeated stimulation of chimeric antigen receptor (CAR) T cells by an antigen. FIG. 3A provides a schematic diagram showing an experimental protocol for repeatedly exposing CAR-T cells to an antigen. The cells were successfully transferred between antigen-coated plates, where each time the cells were contacted with a new antigen-coated plate was considered an individual instance of antigen exposure. Cells were contacted with new antigen-coated plates (i.e., exposed to antigen) between about 4 and 6 times prior to being characterized. In some cases, characterization of the cells involved measuring proliferation, measuring cytotoxicity, phenotyping, and/or measuring cytokine release. FIG. 3B provides a plot showing that CAR-T cells contacted with antigen multiple times according to the method shown in FIG. 3A showed impaired cytotoxicity when co-cultured with target cells (effector to target (E:T) ratio of 1:5) relative to CAR-T cells not exposed to antigen according to the method shown in FIG. 3A (i.e., resting CAR-T cells). FIG. 3C provides a bar graph showing that CAR-T cells contacted with antigen multiple times according to the method shown in FIG. 3A showed reduced proliferative potential with repeated antigen exposures relative to CAR-T cells not exposed to antigen according to the method shown in FIG. 3A (i.e., resting CAR-T cells). In FIGS. 3B and 3C, “T cells” represents T cells that did not express a chimeric antigen receptor (CAR) and that were used to prepare the CAR-T cells evaluated. In FIGS. 3B and 3C the chimeric antigen receptors targeted a cluster of differentiation 19 (CD19) antigen, and the target cells (Nalm6 B cell precursor leukemia cells initiated from an adolescent male and that expressed green fluorescent protein (GFP)) surface-expressed CD19.

FIG. 4 provides a schematic diagram providing lists of representative polypeptides that function as negative regulators of immune cell (e.g., T cell) function, such as polypeptides that are involved in immune cell gene expression regulation, that are involved in immune cell receptor (TCR) or cytokine signaling, that are involved in immune cell growth and/or differentiation, or that are immune cell transcription factors. In embodiments, one or more genes encoding one or more of the polypeptides listed in FIG. 4 may be edited in an immune cell according to the methods provided herein to reduce or eliminate the expression of the encoded polypeptide(s) and to improve one or more characteristics of the immune cell (e.g., efficacy in killing tumor cells).

FIG. 5 provides a schematic diagram showing different types of base edits that may be used to knock-out expression of a polypeptide (i.e., disrupting a start codon, disrupting a splice donor or acceptor site, and/or introduction of a stop codon). In embodiments, a base edit used to reduce or eliminate expression of a polypeptide from a gene is located within a region of the gene corresponding to the first 10%, 25%, 50%, or 75% of the nucleotides transcribed from the gene.

FIG. 6 provides a series of Western blots showing that base editor systems containing the indicated guide polynucleotides (i.e., EF02, EF03, EF04, EF14, EF15, EF01, EF07, EF19, or EF20) (see Tables 1 and 2 for guide sequences) and ABE8.20m were effective in base editing polynucleotides in immune cells to reduce or eliminate expression of the indicated target polypeptides (i.e., CBLB, PTP1B, DNMT3A, CISH, SOCS1, or FLI-1). In FIG. 6, (3-actin was used as a reference.

FIG. 7 provides a plot and images showing that antigen-naïve chimeric antigen receptor (CAR) T cells maintained cytotoxic capacity after being base edited to reduce or eliminate expression of CISH, CBLB, PTP1B, SOCS1, Roquin-1, DNMT3A, FLI-1, Chop, or Regnase-1. In FIG. 7, “T cells” represents T cells that did not express a chimeric antigen receptor (CAR) and that were used to prepare the CAR-T cells evaluated. The immune effector cells (T cells or anti-CD19 CAR-T cells) were co-cultured with target cells (Nalm6 B cell precursor leukemia cells initiated from an adolescent male and that expressed green fluorescent protein (GFP)) at an effector to target cell ratio (E:T) of 1:5 and target cell proliferation was monitored over time by measuring GFP fluorescence/expression. The images of FIG. 7 are fluorescent microscopy images showing that the co-cultures containing the base edited CAR-T cells contained lower levels of tumor cells (i.e., Nalm6 cells) than co-cultures containing T cells (“T cells”) that did not express any chimeric antigen receptor.

FIGS. 8A to 8D provide plots and bar graphs showing that anti-CD19 CAR-T cells base edited to reduce or eliminate expression of CISH, CBLB, PTP1B, SOCS1, Roquin-1, DNMT3A, FLI-1, Chop, or Regnase-1 showed improved antigen-dependent expansion relative to unedited (WT) anti-CD19 CAR-T cells. FIG. 8A provides a plot showing antigen-dependent fold expansion of the indicated anti-CD19 CAR-T cells following antigen exposure at each of the indicated time points. FIG. 8B provides a bar graph showing cumulative fold expansion measurements corresponding to FIG. 8A. FIG. 8C provides a plot showing antigen-independent fold expansion of the indicated anti-CD19 CAR-T cells. FIG. 8D provides a bar graph showing cumulative fold expansion measurements corresponding to FIG. 8C.

FIGS. 9A to 9J provide plots and a bar graph showing that anti-CD19 chimeric antigen receptor (CAR) T cells base edited to reduce or eliminate expression of CISH, CBLB, PTP1B, SOCS1, Roquin-1, DNMT3A, FLI-1, Chop, or Regnase-1 showed higher levels of cytotoxicity after being exposed to antigen 4 times (see FIG. 3A) relative to unedited anti-CD19 CAR-T cells similarly exposed to antigen 4 times. The anti-CD19 CAR-T cells T cells not expressing any CAR were co-cultured with Nalm6 (B cell precursor leukemia cells initiated from an adolescent male) that expressed green fluorescent protein (GFP) at an effector to target cell (E:T) ratio of 1:5. The CAR-T cells were exposed to antigen four (4) times prior to being co-cultured with the target cells (Nalm6 cells). In each of FIGS. 9A to 9J, the base edited cells were edited to reduce or eliminate expression of the polypeptide indicated at the top of the plot. FIG. 9J provides a bar graph showing the area under the curves corresponding to the base edited CAR-T cells of each of FIGS. 9A to 9I, as indicated along the x-axis. In FIG. 9J, “CAR-T” indicates unedited anti-CD19 CAR-T cells. In FIGS. 9A to 9I, the term “GCU” indicates “green calibrated unit,” which is a measure of tumor growth and is proportional to levels of green fluorescent protein expressed by the target Nalm6 tumor cells. In FIGS. 9A to 9J, “T cells” represents T cells that did not express a chimeric antigen receptor (CAR) and that were used to prepare the CAR-T cells evaluated.

FIG. 10 provides a series of plots demonstrating that base editing of anti-CD19 CAR-T cells to reduce or eliminate expression of CISH, SOCS1, or Roquin-1 improved efficacy (e.g., cytotoxicity) of the CAR-T cells following repeated antigen exposures relative to unedited CAR-T cells regardless of the donor from which the CAR-T cells were derived. The anti-CD19 CAR-T cells were prepared using T cells collected from two different human subjects (Donor 1 and Donor 2). The upper plots of FIG. 10 correspond to CAR-T cells prepared using T cells from Donor 1 and the lower plots of FIG. 10 correspond to CAR-T cells prepared using T cells from Donor 2. The CAR-T cells were exposed to antigen four (4) times (see FIG. 3A) prior to being co-cultured with target cells (Nalm6 B cell precursor leukemia cells initiated from an adolescent male and that expressed green fluorescent protein (GFP)) at an effector to target cell ratio (E:T) of 1:5.

FIGS. 11A and 11B provide bar graphs showing that anti-CD19 CAR-T cells base edited according to the methods provided herein to reduce or eliminate expression of CISH, CBLB, SOCS1, Roquin-1, or DNMT3A showed improved T cell intrinsic phenotypes relative to unedited anti-CD19 CAR-T cells after repeated (four) exposures to CD19 antigen. FIG. 11A provides a bar graph showing that anti-CD19 CAR-T cells base edited to reduce or eliminate expression of the polypeptides indicated along the x-axis showed improved proliferation after repeated exposures to antigen, as indicated by lower-magnitude fold-changes in instances of the Ki67+ phenotype in the base edited CAR-T cells. The fold change in Ki67+ expression was measured relative to the cells prior to repeated exposures to antigen. In FIG. 11A, a higher negative value indicates less proliferation. FIG. 11B provides a bar graph showing reduced levels of the phenotype EOMES+, T-bet− in populations of anti-CD19 CAR-T cells base edited to reduce or eliminate expression of the polypeptides indicated along the x-axis after repeated antigen exposures. The phenotype EOMES+, T-bet− is associated with an exhausted phenotype (e.g., reduced cytotoxicity, reduced proliferation, reduced cytokine production, reduced survival, increase in inhibitory receptors, and/or upregulation of repressor genes) in T cells. In FIGS. 11A and 11B, “CAR-T” indicates anti-CD19 CAR-T cells that were not base edited. In FIGS. 11A and 11B, the anti-CD19 CAR-T cells were exposed to CD19 antigen four (4) times (see FIG. 3A).

FIGS. 12A to 12D provide bar graphs showing that anti-CD19 CAR-T cells base edited according to the methods provided herein to reduce or eliminate expression of CISH, SOCS1, or Roquin-1 maintained robust cytokine (e.g., GZMB, IL-2, IFNg, TNFa) secretion after repeated antigen exposures. The CAR-T cells were exposed to antigen four times (see FIG. 3A) prior to co-culturing the cells with target cells and subsequently measuring, in pg/mL, secretion of the cytokines granzyme B (GZMB; FIG. 12A), interferon gamma (IFNg; FIG. 12B), interleukin-2 (IL-2; FIG. 12C), and tumor necrosis factor alpha (TNFa; FIG. 12D). The CAR-T cells were co-cultured with target cells (Nalm6 B cell precursor leukemia cells initiated from an adolescent male and that expressed green fluorescent protein (GFP)) at an effector to target cell ratio (E:T) of 1:1. In FIGS. 12A to 12D, unedited CAR-T cells (CAR-T), unedited CAR-T cells cultured in the absence of target cells (CAR-T Alone), and target cells cultured in the absence of any CAR-T cells (Tumor Alone) were evaluated as controls. In each of FIGS. 12A to 12D, the dashed horizontal lines indicate levels of secretion measured for unedited CAR-T cells co-cultured with the target cells.

FIGS. 13A to 13D provide plots showing that base editing of anti-CD19 CAR-T cells according to the methods provided herein to reduce or eliminate expression of CISH, CBLB, SOCS1, Roquin-1, DNMT3A, FLI-1, CHOP, or Regnase-1 improved antitumor activity of the cells in vivo relative to unedited CAR-T cells. Anti-tumor activity of the CAR-T cells was evaluated using a sub-therapeutic B cell lymphoma (BCL) model involving the infusion of 5E5 Raji cells (a human B lymphoblastoid cell line derived from a patient with Burkitt lymphoma) expressing luciferase into mice at day zero (0) and subsequently infusing 1E6 CAR-T cells into the mice at day 1. FIGS. 13A to 13C provide plots showing measurements over time of tumor growth, measured as luciferase flux in photons per second (p/s), in mice administered the Raji cells and treated using anti-CD19 CAR-T cells base edited to knock-out (KO) expression of SOCS1, DNMT3A, or FLI-1, respectively. FIG. 13D provides a plot showing measurements over time of tumor growth, measured as luciferase flux in photons per second (p/s), in mice administered the Raji cells and treated using anti-CD19 CAR-T cells base edited to knock-out (KO) expression of CISH, CBLB, SOCS1, Roquin-1, DNMT3A, FLI-1, CHOP, or Regnase-1, as indicated. Knock out of FLP-1 expression, in particular, was effective in delaying tumor progression. In FIGS. 13A to 13D, the term “LV224” indicates unedited anti-CD19 CAR-T cells, the term “UTD,” which is short for “untransduced,” indicates T cells from which the CAR-T cells were derived but that do not express any chimeric antigen receptor (CAR), the term “Treatment” indicates the time at which mice were administered the CAR-T cells, and “Days Post Implant” indicates time in days measured from administration of the Raji cells to the mice.

FIGS. 14A and 14B provide a plot and images showing that base editing of anti-CD19 CAR-T cells according to the methods provided herein to reduce or eliminate expression of CISH, CBLB, SOCS1, Roquin-1, DNMT3A, FLI-1, RASA2, Regnase-1, CD5, or PTP1B improved antitumor activity of the cells in vivo relative to unedited CAR-T cells. Anti-tumor activity of the CAR-T cells was evaluated using a sub-therapeutic mantle cell lymphoma (MCL) model involving the infusion of 5E5 JeKo-1 cells (a mantle cell lymphoma (MCL) cell line isolated from the peripheral blood mononuclear cells of a 78-year-old female with a large cell variant of MCL showing leukemic conversion) expressing luciferase into mice at day zero (0) and subsequently infusing 2.5E5 CAR-T cells into the mice at day 7. FIG. 14A provides a plot showing measurements over time of tumor growth, measured as luciferase flux in photons per second (p/s), in mice administered the JeKo-1 cells and treated using anti-CD19 CAR-T cells base edited to knock-out (KO) expression of Roquin-1. The chimeric antigen receptor (CAR) polypeptides expressed by the anti-CD19 CAR-T cells contained an anti-CD19 scFv as an antigen-binding domain, a 4-1BB costimulatory domain, and a CD3ζ signaling domain. Knock out of Roquin-1 expression in the anti-CD19 CAR-T cells prolonged tumor control in the MCL model. FIG. 14B provides bioluminescence images of mice taken at about day 45 following administration of the JeKo-1 cells, where the mice were treated using anti-CD19 CAR-T cells base edited to knock-out (KO) expression of CISH, CBLB, SOCS1, Roquin-1, DNMT3A, FLI-1, RASA2, Regnase-1, CD5, or PTP1B. In FIG. 14B, tumor-free regions of the mice are shown as lighter areas (i.e., areas of low bioluminescence). In FIGS. 14A and 14B, the terms “LV224” and “WT” indicate unedited anti-CD19 CAR-T cells, the term “UTD,” which is short for “untransduced,” indicates T cells from which the CAR-T cells were derived but that do not express any chimeric antigen receptor (CAR), the term “EF08” indicates the guide polynucleotide (see Tables 1 and 2) used to base edit cells to reduce or eliminate expression of Roquin-1, and “Days Post Inoculation” indicates time in days measured from administration of the JeKo-1 cells to the mice.

FIGS. 15A to 15C provide bar graphs showing percent (%) on-target base editing efficiencies measured in immune cells for base editor systems containing the guides indicated along the x-axis (e.g., EF46; see also Tables 1 and 2). In FIGS. 15A and 15B, each guide polynucleotide name is followed by an underscore (“_”) and the name of the polypeptide encoded by the polynucleotide targeted by the guide polynucleotide. In FIG. 15C, the polypeptide encoded by the polynucleotide targeted by each guide polynucleotide is indicated below each guide polynucleotide. In FIG. 15C, a guide known to be effective in targeting a base editor to edit beta microglobulin (B2M) was used as a positive control. Many of the base editor systems evaluated had percent on-target base editing efficiencies of over about 80%. Knock-out of polypeptide expression was validated using Western blots.

FIGS. 16A and 16B provide a flow cytometry contour plot and a stacked bar graph showing that base editing of anti-CD19 CAR-T cells to reduce or eliminate expression of DCK, DGKa, DGKz, PRDM1 (BLIMP-1), PRKACA, PTPN6, EIF2A, ID3, IKZF2, SOX4, TLE4, TMEM184B, CD5, RASA2, DHX37, PFN1, BATF, or ARID1A altered CD4/CD8 CAR-T cell ratios of cells after multiple exposures to antigen relative to unedited CAR-T cells (“WT”). Prior to measuring CD4/CD8 cell ratios using flow cytometry, the CAR-T cells were stimulated four times by being exposed to antigen (see FIG. 3A) and then co-cultured overnight with Nalm6 cells (B cell precursor leukemia cells initiated from an adolescent male) at an effector to target cell (E:T) ratio of 1:1. FIG. 16A provides a representative flow cytometry contour plot showing a gating strategy for distinguishing CD4+ cells from CD8+ cells. FIG. 16B provides a stacked bar graph showing percent CD4+ and CD8+ cells measured in CAR-T cells base edited to knock out expression of the polypeptides indicated along the x-axis. The arrows in FIG. 16B indicate cell populations that had high levels of CD4+ cells.

FIGS. 17A and 17B provide bar graphs showing the percents of the CD8+ (FIG. 17A) and CD4+ (FIG. 17B) CAR-T cell populations of FIG. 16B that were also positive for expression of cluster of differentiation 25 (CD25), which is an indicator of T cell activation. Base edited CAR-T cells showed enhanced levels of activation. The dashed horizontal lines in FIGS. 17A and 17B indicate the percent CD25+ cells measured in unedited cells (“19BBz (no edit)”). The term “19BBz” refers to the anti-CD19 scFv antigen-binding domain, 4-1BB costimulatory domain, and CD3ζ signaling domain of the chimeric antigen receptor (CAR) expressed by the CAR-T cells.

FIGS. 18A and 18B provide a plot and a bar graph showing that base editing of anti-CD19 CAR-T cells to reduce or eliminate expression of DCK, DGKa, DGKz, PRDM1 (BLIMP-1), PRKACA, PTPN6, EIF2A, ID3, IKZF2, SOX4, TLE4, TMEM184B, CD5, RASA2, DHX37, PFN1, BATF, or ARID1A enhanced proliferation of the cells after multiple antigen-exposures relative to unedited CAR-T cells (“WT”). Prior to measuring cell proliferation, the CAR-T cells were stimulated four times by being exposed to antigen (see FIG. 3A). All of the CAR-T cells were prepared using T cells collected from the same donor subject. FIG. 18A provides a plot showing fold expansion of the cells over time. FIG. 18B provides a bar graph showing cumulative fold expansion values corresponding to the “full expansion curves” of FIG. 18A.

FIGS. 19A to 19C provide plots showing that base editing of anti-CD19 CAR-T cells according to the methods provided herein to reduce or eliminate expression of DCK, CD5, DGKα/ζ (DGKa and DGKz), DHX37, EIF2A, ID3, IKZF2, IL-6, PFN1, PRDM1, PRKACA, PTPN6, RASA2, SOX4, TLE, TMEM184B, or TMEM222 improved cytotoxicity of the cells after multiple antigen exposures relative to unedited CAR-T cells. Prior to measuring cytotoxicity, the CAR-T cells were stimulated four times by being exposed to CD19 antigen (see FIG. 3A). To measure cytotoxicity, the cells were co-cultured overnight with Nalm6 cells (B cell precursor leukemia cells initiated from an adolescent male) expressing green fluorescent protein (GFP) at an effector to target cell (E:T) ratio of 1:5. Clearance of the Nalm6 cells was measured over time using live cell fluorescent imaging carried out using an IncuCyte® Live-Cell Analysis System. In FIG. 19A, the cells were base edited using one of the following guides (see Tables 1 and 2A): EF46, EF58, EF74, EF76, EF85, EF86, EF91, EF112, EF114, or EF117. In FIG. 19C, the cells were base edited using one of the following guides (see Tables 1 and 2A): EF46, EF58, EF70, EF74, EF76, EF85, EF86, EF91, EF112, EF114, or EF117. In FIGS. 19A to 19C, “GCU” indicates “green calibrated unit,” which is a measure of tumor growth and is proportional to levels of green fluorescent protein expressed by the target Nalm6 tumor cells, “UTD,” which is short for “untransduced,” indicates T cells from which the CAR-T cells were derived but that do not express any chimeric antigen receptor (CAR), and “LV224” and “19BBz” indicate unedited CAR-T cells. The term “19BBz” refers to the anti-CD19 scFv antigen-binding domain, 4-1BB costimulatory domain, and CD3ζ signaling domain of the anti-CD19 chimeric antigen receptor (CAR) expressed by the CAR-T cells.

FIGS. 20A to 20D provide plots showing that base editing of anti-CD19 CAR-T cells according to the methods provided herein to reduce or eliminate expression of cluster of differentiation 5 (CD5) improved cytotoxicity of the cells after multiple (6 total) CD19 antigen exposures relative to unedited CAR-T cells similarly exposed to the CD19 antigen multiple times. The T cells were co-cultured with target cells at effector to target cell ratios (E:T) of 1:1, 1:2, 1:4, and 1:8, as indicated along the x-axes of FIGS. 20A to 20D, and lysis of the target cells was evaluated after 24 hours and 48 hours of coculture. Prior to being co-cultured with target cells, the T cells were exposed to CD19 antigen six times (see FIG. 3A). The target cells were either JeKo-1 cells (a mantle cell lymphoma (MCL) cell line isolated from the peripheral blood mononuclear cells of a 78-year-old female with a large cell variant of MCL showing leukemic conversion) or Nalm6 cells (B cell precursor leukemia cells initiated from an adolescent male). FIG. 20A provides a plot showing percent specific cell lysis of Nalm6 target cells after 24 hours of co-culture. FIG. 20B provides a plot showing percent specific cell lysis of Nalm6 target cells after 48 hours of co-culture. FIG. 20C provides a plot showing percent specific cell lysis of JeKo-1 target cells after 24 hours of co-culture. FIG. 20D provides a plot showing percent specific cell lysis of JeKo-1 target cells after 48 hours of co-culture. In FIGS. 20A to 20D, the term “UTD,” which is short for “untransduced,” indicates T cells from which the CAR-T cells were derived but that do not express any chimeric antigen receptor (CAR), “6 Ag-Exp” indicates “six antigen exposures,” and “19BBz” indicates unedited CAR-T cells. In FIGS. 20A to 20D, a negative “% specific lysis” indicates that the target cells showed net proliferation during the co-culture so that there were more target cells at the end of co-culture than at the beginning.

FIGS. 21A and 21B provide plots demonstrating that knock-out (KO) of Roquin-1 expression in chimeric antigen receptor (CAR) expressing T cells (CAR-T cells) was associated with improved cytotoxicity in vitro independent of the co-stimulatory domain of the chimeric antigen receptor. The CAR polypeptides contained an anti-CD19 scFv as an antigen-binding domain, either a 4-1BB costimulatory domain or a CD28 co-stimulatory domain, and a CD3ζ signaling domain. Cytotoxicity of the CAR-T cells was measured using an IncuCyte® Live-Cell Analysis System following being exposed to antigen 6 times according to the method shown in FIG. 3A. The CAR-T cells were co-cultured with JeKo-1 lymphoblast cells expressing green fluorescent protein (GFP) at an effector-to-target ratio (E:T) of 1:5, where the CAR-T cells were the effector cells and the JeKo-1 cells were the target cells. FIG. 21A provides a plot showing improved cytotoxicity of Roquin-1 KO CAR-T cells relative to unedited CAR-T cells, where the CAR polypeptides contained a R-1BB co-stimulatory domain. FIG. 21B provides a plot showing improved cytotoxicity of Roquin-1 KO CAR-T cells relative to unedited CAR-T cells, where the CAR polypeptides contained a CD28 co-stimulatory domain. In FIGS. 21A and 21B the term “19BBz” refers to T cells expressing Roquin-1 and a CAR polypeptide containing an anti-CD19 scFv antigen-binding domain, 4-1BB costimulatory domain, and CD3ζ signaling domain; the term “1928z” refers to T cells expressing Roquin-1 and a CAR polypeptide containing an anti-CD19 scFv antigen-binding domain, CD28 costimulatory domain, and CD3ζ signaling domain; the term “Roquin-1 KO” indicates CAR-T cells modified to knock out expression of Roquin-1; the term “T cells” indicates T cells that express Roquin-1 and do not express any CAR; and the term “GCU” indicates a Green calibrated unit (GCU).

FIGS. 22A to 22C provide plots demonstrating, when compared, e.g., to FIGS. 21A and 21B, that knock-out (KO) of Roquin-1 expression in chimeric antigen receptor (CAR) expressing T cells (CAR-T cells) lead to an increase in cytotoxicity of CAR-T cells across alternative scFv domains. The CAR polypeptides contained an anti-CD22 scFv domain, a 4-1BB costimulatory domain, and a CD3ζ signaling domain. The CAR-T cells were co-cultured with antigen-positive Nalm-6 tumor cells expressing GFP at effector-to-target ratios (E:T) of 1:2 (FIG. 22A), 1:5 (FIG. 22B), and 1:10 (FIG. 22C), where the effector cells were the CAR-T cells and the target cells were the Nalm-6 tumor cells expressing GFP. Cytotoxicity of the CAR-T cells was measured using an IncuCyte® Live-Cell Analysis System following being exposed to antigen 4 times according to the method shown in FIG. 3A. In FIGS. 22A to 22C, the term “22BBz CAR cells” refers to T cells expressing Roquin-1 and a CAR polypeptide containing an anti-CD22 scFv antigen-binding domain, 4-1BB costimulatory domain, and CD3ζ signaling domain; the term “Roquin-1 KO” indicates CAR-T cells modified to knock out expression of Roquin-1; the term “Tumor only” indicates Nalm-6 tumor cells cultured in the absence of any effector cells; and the term “GCU” indicates a Green calibrated unit (GCU).

FIG. 23 provides a bar graph demonstrating that Roquin-1 knock-out (KO) chimeric antigen receptor (CAR) expressing T cells (CAR-T cells) expressing two different chimeric antigen receptors showed improved anti-tumor activity over similar CAR-T cells expressing Roquin-1. The CAR-T cells expressed two CAR polypeptides where one CAR polypeptide contained an anti-CD19 scFv domain, a 4-1BB costimulatory domain, and a CD3ζ signaling domain and the other CAR polypeptide contained an anti-ROR1 scFv domain, a CD28 costimulatory domain, and a CD3ζ signaling domain. The CAR-T cells were also editing according to the methods provided herein to knock-out expression of one of the following polypeptides prior to evaluating cytotoxicity (see x-axis of FIG. 23): CISH, SOCS1, Roquin-1, DNMT3A, FLI1, Regnase-1, DGKa, DGKz, PRKACA, PTPN6, EIF2A, RASA2, or DHX37. The CAR-T cells, which were not previously contacted with a target antigen, were co-cultured with JeKo-1 lymphoblast cells expressing GFP at an effector-to-target ratio (E:T) of 1:5, where the CAR-T cells were the effector cells and the JeKo-1 cells were the target cells. Cytotoxicity of the CAR-T cells was measured using an IncuCyte® Live-Cell Analysis System. FIG. 23 provides a bar graph showing the area under the curve (AUC) for plots of tumor levels over time, measured as green calibrated units (GCU), for the co-cultures. In FIG. 23 the terms “T cells” and “UTD” indicate T cells without expression of any of the above-listed polypeptides knocked out and that do not express a CAR (i.e., untransduced (UTD) cells); the terms “WT aROR1/CD19 CAR” and “WT Dual CAR” indicate CAR-T cells expressing the two CAR polypeptides and without expression of any of the above-listed polypeptides knocked out; and the term “Roquin-1 KO” indicates CAR-T cells altered to knock out expression of Roquin-1.

FIGS. 24A to 24C each provide a plot presenting data from FIGS. 14A and 14B in an alternative format and demonstrating that knock-out of Roquin-1 increased the ability of CAR-T cells to kill tumor cells in the mantle cell lymphoma (MCL) mouse model. FIG. 24C provides a plot where data corresponding to Roquin-1 knock-out responders (2 out of 10 (8/10) total mice) are plotted separately from data corresponding to Roquin-1 knock-out non-responders (2 out of 10 (2/10) total mice), where “responder” indicates a mouse where administration of Roquin-1 knock-out (KO) chimeric antigen receptor (CAR) expressing T cells (CAR-T cells) led to an increased reduction in tumor levels in the mice relative to administration of CAR-T cells expressing Roquin-1. In FIGS. 24A to 24C, the term “T cells” indicates mice administered T cells that were not edited to knock-out expression of CISH, CBLB, SOCS1, Roquin-1, DNMT3A, FLI-1, RASA2, Regnase-1, CD5, or PTP1B and that did not express any CAR polypeptide; the term “19BBz CAR-T cells” indicates mice administered CAR-T cells that were not edited to knock-out expression of CISH, CBLB, SOCS1, Roquin-1, DNMT3A, FLI-1, RASA2, Regnase-1, CD5, or PTP1B; the terms “SOCS1 KO,” “FLI-1 KO,” “Roquin-1 KO,” and “CBLB KO” indicate mice administered CAR-T cells edited to knock-out expression of the indicated polypeptide; the term “Roquin-1 KO (responders)” indicates mice where administration of Roquin-1 KO CAR-T cells led to a reduction in tumor levels in the mice relative to mice administered CAR-T cells expressing Roquin-1; the term “Roquin-1 KO (non-responders)” indicates mice where administration of Roquin-1 KO CAR-T cells did not lead to a significant reduction in tumor levels in the mice relative to mice administered CAR-T cells expressing Roquin-1.

FIGS. 25A and 25B provide plots showing that Roquin-1 knock-out (KO) chimeric antigen receptor (CAR) T cells cleared tumors in vivo and were associated with improved prevention of tumor recurrence relative to CAR-T cells expressing Roquin-1. The CAR-T cells expressed a CAR polypeptide containing an anti-CD19 scFv as an antigen-binding domain, a 4-1BB costimulatory domain, and a CD3ζ signaling domain. Anti-tumor activity of the CAR-T cells was evaluated using a sub-therapeutic (FIG. 25A) or tumor clearance and rechallenge (FIG. 25B) mantle cell lymphoma (MCL) model involving the infusion (“inoculation”) of 5E5 JeKo-1 cells (a mantle cell lymphoma (MCL) cell line isolated from the peripheral blood mononuclear cells of a 78-year-old female with a large cell variant of MCL showing leukemic conversion) expressing luciferase into mice at day zero (0) and subsequently infusing 2.5E5 CAR-T cells (FIG. 25A; “low dose”) or 2.5E6 CAR-T cells (FIG. 25B; “high dose”) into the mice at day 7. The plots of FIGS. 26A and 25B show measurements over time of tumor growth, measured as luciferase flux in photons per second (p/s) in the mice. The CAR-T cells were edited according to the methods provided herein to knock-out expression of DGKz, FLI-1, or Roquin-1. In FIGS. 25A and 25B, the term “T cells” indicates mice administered T cells that were not edited to knock-out expression of DGKz, FLI-1, or Roquin-1 and that did not express any CAR polypeptide; the term “19BBz CAR-T cells” indicates mice administered CAR-T cells that were not edited to knock-out expression of DGKz, FLI-1, or Roquin-1; the terms “DGKz KO,” “FLI-1 KO,” and “Roquin-1 KO” indicate mice administered CAR-T cells edited to knock-out expression of the indicated polypeptide; and the term “Tumor only” indicates mice that were not administered any effector cells (e.g., CAR-T cells or T cells). In FIG. 25B, the vertical dotted line indicates the time at which the mice were re-administered the JeKo-1 cells.

FIGS. 26A and 26B provide a plot and bar graph that, when compared to FIGS. 25A and 25B, show that knock-out (KO) of Roquin-1 in chimeric antigen receptor (CAR) expressing T cells (CAR-T cells) enhanced efficacy of the CAR-T cells independent of the co-stimulatory domain of the CAR polypeptide. The CAR-T cells expressed a CAR polypeptide containing an anti-CD19 scFv as an antigen-binding domain, a CD28 costimulatory domain, and a CD3ζ signaling domain. Anti-tumor activity of the CAR-T cells was evaluated using a sub-therapeutic mantle cell lymphoma (MCL) model involving the infusion (“inoculation”) of 5E5 JeKo-1 cells (a mantle cell lymphoma (MCL) cell line isolated from the peripheral blood mononuclear cells of a 78-year-old female with a large cell variant of MCL showing leukemic conversion) expressing luciferase into mice at day zero (0) and subsequently infusing 2.5E5 CAR-T cells into the mice at day 7. FIG. 26A shows measurements over time of tumor growth, measured as luciferase flux in photons per second (p/s) in the mice, and FIG. 26B provides a bar graph showing the area under the curve (AUC) for each curve of FIG. 26A. The CAR-T cells were edited according to the methods provided herein to knock-out expression of DGKz, FLI-1, or Roquin-1. In FIGS. 25A and 25B, the term “T cells” indicates mice administered T cells that were not edited to knock-out expression of DGKz, FLI-1, or Roquin-1 and that did not express any CAR polypeptide; the term “1928z CAR-T cells” indicates mice administered CAR-T cells that were not edited to knock-out expression of DGKz, FLI-1, or Roquin-1; and the terms “DGKz KO,” “FLI-1 KO,” and “Roquin-1 KO” indicate mice administered CAR-T cells edited to knock-out expression of the indicated polypeptide.

FIG. 27 provides a schematic diagram summarizing an experiment undertaken to evaluate expansion kinetics and cytokine secretion of chimeric antigen receptor (CAR) expression T cells (CAR-T cells) of the disclosure in vivo. Anti-tumor activity of the CAR-T cells was evaluated using a mantle cell lymphoma (MCL) model involving the infusion (“inoculation”) of 5E5 JeKo-1 cells (a mantle cell lymphoma (MCL) cell line isolated from the peripheral blood mononuclear cells of a 78-year-old female with a large cell variant of MCL showing leukemic conversion) expressing luciferase into mice at day zero (−7) and subsequently infusing CAR-T cells into the mice at day 0. The CAR-T cells expressed a CAR polypeptide containing an anti-CD19 scFv as an antigen-binding domain, a 4-1BB costimulatory domain, and a CD3ζ signaling domain. Bleed samples were collected from the mice twice a week, and tumor growth was evaluated once a week using an IVIS® Spectrum In Vivo Imaging System. Serum was collected from the mice to measure cytokines, and the blood was processed to quantify CAR-T cell counts using flow cytometry (CAR/EGFR detection+phenotyping (CD45RA, CD62L)). The following cytokines were measured: IL-2, IL-6, TNFα, IFNγ, GM-CSF, and GRZB. The terms “Group A” and “Group B” in FIG. 27 refer to the mice corresponding to Group 1A, 2A, 3A, 4A, 5A, and/or 6A or to Group 1B, 2B, 3B, 4B, 5B, and/or 6B described in Table 9, respectively.

FIG. 28 provides a set of plots showing data corresponding to the experiment described in FIG. 27 and demonstrating that peak CAR-T cell expansion was detected at between 7 and 10 days post-administration of the CAR-T cells. In FIG. 28, the term “low dose” indicates mice administered 5E+5 CAR-T cells; the term “high dose” indicates mice administered 2.5E+6 CAR-T cells; the terms “DGKz” and “Roquin-1” indicate mice administered T cells edited to knock-out expression of the indicated polypeptide; and the term “LV224” indicates CAR-T cells that were not edited to knock out expression of DGKz or Roquin-1.

FIG. 29 provides a set of plots showing data corresponding to the experiment described in FIG. 27 and demonstrating that CAR-T cell expansion kinetics correlated with detected tumor burden in vivo. In FIG. 29, the term “low dose” indicates mice administered 5E+5 CAR-T cells; the term “high dose” indicates mice administered 2.5E+6 CAR-T cells; the terms “DGKz” and “Roquin-1” indicate mice administered T cells edited to knock-out expression of the indicated polypeptide; the term “LV224” indicates CAR-T cells that were not edited to knock out expression of DGKz or Roquin-1; the term “CAR Expansion” indicates CAR-T cell counts in blood samples measured over time; and the term “Mouse BLI” indicates measurements over time of tumor growth, measured using in vivo bioluminescent imaging (BLI) as luciferase flux in photons per second (p/s) in the mice. In FIG. 29, the “CAR Expansion” curve corresponds to the right y-axis and the “Mouse BLI” curve corresponds to the left y-axis.

FIG. 30 provides a bar graph showing data corresponding to the experiment described in FIG. 27 and demonstrating that Roquin-1 knock-out (KO) CAR-T cells maintained the central memory phenotype over time. In FIG. 30, the term “CAR T cells” indicates CAR-T cells that were not edited to knock out expression of DGKz or Roquin-1; and the term “CAR T+Roquin-1 KO” indicates CAR-T cells modified accordingly to the methods provided herein to knock-out expression of Roquin-1.

FIG. 31 provides a schematic diagram summarizing a mechanism of action for chimeric antigen receptor (CAR) expressing T cells.

FIG. 32 provides a schematic diagram showing how Roquin-1 regulates mRNA degradation.

FIGS. 33A to 33D provide bar graphs and flow cytometry histograms demonstrating that Roquin-1 knock-out in CAR-T cells was associated with an increase in expression in the CAR-T cells of the activation markers OX40 and CD25. The CAR-T cells expressed a CAR polypeptide containing an anti-CD19 scFv as an antigen-binding domain, a 4-1BB costimulatory domain, and a CD3ζ signaling domain. The CAR-T cells were co-cultured for 24-hours with JeKo-1 cells (a mantle cell lymphoma (MCL) cell line isolated from the peripheral blood mononuclear cells of a 78-year-old female with a large cell variant of MCL showing leukemic conversion). The CAR-T cells were co-cultured at an effector-to-target ratio of 1:1 with the JeKo-1 cells following being exposed to antigen 4 times according to a method similar to that shown in FIG. 3A with the modification that the cells were contacted with beads coated with the antigen targeted by the CAR T cells rather than with plates coated with the antigen. FIG. 33A provides a plot showing percent of total CD4+ or CD8+ CAR-T cells expressing OX40. FIG. 33B provides a flow cytometry histogram showing levels of OX40 expression in CD4+ CAR-T cells. FIG. 33C provides a bar graph showing geometric mean of fluorescence intensity (GMRI) measuring levels of CD25 expression in CD4+ or CD8+ CAR-T cells. Each set of four bars in FIGS. 33A and 33C correspond from left-to-right to “Roquin1,” “FL1,” “DGKz,” and “WT,” respectively. FIG. 33D provides flow cytometry histograms showing levels of CD25 expression in CD8+ CAR-T cells. The flow cytometry histograms in each of FIGS. 33B and 33D correspond from top-to-bottom to “Roquin1,” “FLI,” “DGKz,” and “WT,” respectively. In FIGS. 33A to 33D “Roquin1,” “FLI1,” and “DGKz” indicate CAR-T cells modified according to the methods provided herein to knock-out expression of Roquin-1, FLI-1, or DGKz, respectively; and the term “WT” indicates CAR-T cells expressing each of Roquin-1, FLI-1, and DGKz.

FIGS. 34A to 34D provide bar graphs and flow cytometry histograms demonstrating that Roquin-1 knock-out in CAR-T cells was associated with increased expression of T cell co-stimulatory signals. The CAR-T cells expressed a CAR polypeptide containing an anti-CD19 scFv as an antigen-binding domain, a 4-1BB costimulatory domain, and a CD3ζ signaling domain. The CAR-T cells were co-cultured for 24-hours with JeKo-1 cells (a mantle cell lymphoma (MCL) cell line isolated from the peripheral blood mononuclear cells of a 78-year-old female with a large cell variant of MCL showing leukemic conversion). The CAR-T cells were co-cultured at an effector-to-target ratio of 1:1 with the JeKo-1 cells following being exposed to antigen 4 times according to the method shown in FIG. 3A. FIG. 34A provides a plot showing geometric mean of fluorescence intensity (GMRI) measuring levels of expression of ICOS in CD4+ CAR-T cells. FIG. 34B provides a flow cytometry histogram showing levels of ICOS expression in CD4+ CAR-T cells. FIG. 34C provides a bar graph showing percent of total CD4+ or CD8+ CAR-T cells expressing CD28. Each set of four bars in FIGS. 34A and 34C correspond from left-to-right to “Roquin1,” “FL1,” “DGKz,” and “WT,” respectively. FIG. 33D provides flow cytometry histograms showing levels of CD28 expression in CD8+ CAR-T cells. The flow cytometry histograms in each of FIGS. 34B and 34D correspond from top-to-bottom to “Roquin1,” “FL1,” “DGKz,” and “WT,” respectively. In FIGS. 34A to 34D “Roquin1,” “FLIT,” and “DGKz” indicate CAR-T cells modified according to the methods provided herein to knock-out expression of Roquin-1, FLI-1, or DGKz, respectively; and the term “WT” indicates CAR-T cells expressing each of Roquin-1, FLI-1, and DGKz.

FIGS. 35A to 35C provide bar graphs and flow cytometry scatter plots demonstrating that Roquin-1 knock-out in CAR-T cells was associated with a large increase in IL-2 secretion compared to CAR-T cells expressing Roquin-1. The CAR-T cells expressed a CAR polypeptide containing an anti-CD19 scFv as an antigen-binding domain, a 4-1BB costimulatory domain, and a CD3ζ signaling domain. The CAR-T cells were co-cultured at an effector-to-target ratio of 1:1 for 24-hours with Raji cells (a line of lymphoblast-like cells isolated in 1963 from the jaw of a Burkitt's lymphoma patient). The CAR-T cells were co-cultured with the Raji cells following being exposed to antigen 1 or 5 times (e.g., 5 times for FIGS. 35B and 35C) according to the method shown in FIG. 3A. FIG. 35A provides a bar graph showing measured levels of secreted IL-2 for CAR-T cells exposed to antigen once (1×) or five times (5×) prior to co-culture. FIG. 35B provides a bar graph showing geometric mean of fluorescence intensity (GMRI) measuring levels of intracellular expression of IL-2 for CD4+ or CD8+ CAR-T cells. Each set of four bars in FIGS. 35A and 35B correspond from left-to-right to “Roquin1,” “FL1,” “DGKz,” and “WT,” respectively. FIG. 35C provides flow cytometry scatter plots showing counts of CD4+ CAR-T cells expressing IL-2 intracellularly, where the percent of total cells counted falling within each outlined region is indicated above each respective outlined region. In FIGS. 35A to 35C, “Roquin1,” “FLIT,” and “DGKz” indicate CAR-T cells modified according to the methods provided herein to knock-out expression of Roquin-1, FLI-1, or DGKz, respectively; and the term “WT” indicates CAR-T cells expressing each of Roquin-1, FLI-1, and DGKz.

FIGS. 36A to 36C provide bar graphs and flow cytometry scatter plots demonstrating that Roquin-1 knock-out in CAR-T cells was associated with a increases in interferon gamma (IFNγ) secretion compared to CAR-T cells expressing Roquin-1. The CAR-T cells expressed a CAR polypeptide containing an anti-CD19 scFv as an antigen-binding domain, a 4-1BB costimulatory domain, and a CD3ζ signaling domain. The CAR-T cells were co-cultured at an effector-to-target ratio of 1:1 for 24-hours with Raji cells (a line of lymphoblast-like cells isolated in 1963 from the jaw of a Burkitt's lymphoma patient). The CAR-T cells were co-cultured with the Raji cells following being exposed to antigen 1 or 5 times (e.g., 5 times for FIG. 36C) according to the method shown in FIG. 3A. FIG. 36A provides a bar graph showing measured levels of secreted IFNγ for CAR-T cells exposed to antigen once (1×) or five times (5×) prior to co-culture. FIG. 36B provides a bar graph showing percent of total CD4+ or CD8+ CAR-T cells intracellularly expressing IFNγ. Each set of four bars in FIGS. 36A and 36B correspond from left-to-right to “Roquin1,” “FL1,” “DGKz,” and “WT,” respectively. FIG. 36C provides flow cytometry scatter plots showing counts of CD8+ and CD4+ CAR-T cells expressing IL-2 and IFNγ intracellularly, where the percent of total cells counted falling within each quadrant is indicated by a number within the respective quadrant. In FIG. 36A, “Roquin1,” “FLI1,” and “DGKz” indicate CAR-T cells modified according to the methods provided herein to knock-out expression of Roquin-1, FLI-1, or DGKz, respectively; and the term “WT” indicates CAR-T cells expressing each of Roquin-1, FLI-1, and DGKz.

FIGS. 37A and 37B provide bar graphs and flow cytometry scatter plots showing TNF-α in CAR-T cells modified to knock out expression of Roquin-1 (Roquin1), FLI-1, or DGKZ. The CAR-T cells expressed a CAR polypeptide containing an anti-CD19 scFv as an antigen-binding domain, a 4-1BB costimulatory domain, and a CD3ζ signaling domain. The CAR-T cells were co-cultured for 24-hours with Raji cells (a line of lymphoblast-like cells isolated in 1963 from the jaw of a Burkitt's lymphoma patient). The CAR-T cells were co-cultured at an effector-to-target ratio of 1:1 with the Raji cells following being exposed to antigen 1 or 5 times according to the method shown in FIG. 3A. FIG. 37A provides a bar graph showing measured levels of secreted TNF-α for CAR-T cells exposed to antigen once (1×) prior to co-culture. FIG. 37B provides a bar graph showing measured levels of secreted TNF-α for CAR-T cells exposed to antigen five times (5×) prior to co-culture. In FIGS. 37A and 37B, “Roquin1,” “FLI1,” and “DGKz” indicate CAR-T cells modified according to the methods provided herein to knock-out expression of Roquin-1, FLI-1, or DGKz, respectively; and the term “WT” indicates CAR-T cells expressing each of Roquin-1, FLI-1, and DGKz.

### DETAILED DESCRIPTION OF THE DISCLOSURE

The disclosure features multiplex base edited chimeric antigen receptor (CAR)-expressing immune effector cells (e.g., T or NK cells) having increased resistance to development of an exhausted phenotype (e.g., increased cytotoxicity, proliferation, survival, and/or cytokine production) after repeated or continuous stimulation by an antigen relative to unedited CAR immune effector cells, compositions containing the cells, methods for the preparation of the cells, and methods for use of the cells in treating a disease or disorder (e.g., an autoimmune disorder or a neoplasia, such as a leukemia).

The disclosure is based, at least in part, on the discovery that chimeric antigen receptor (CAR) T cells base edited to reduce or eliminate expression of Roquin-1, CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, or TMEM222 had a reduced tendency relative to unedited CAR-T cells to develop an exhausted phenotype after being stimulated by multiple antigen exposures or continuous exposure to an antigen. The base edited CAR-T cells showed increased efficacy (e.g., increased cytotoxicity, increased proliferation, and/or increased cytokine production) relative to unedited CAR-T cells. In various embodiments, the improved efficacy of the CAR-T cells and/or reduced tendency to develop the exhausted phenotype is independent of tumor composition and/or phenotype in a subject administered the CAR-T cells.

Accordingly, the disclosure provides multiplex base edited CAR-expressing immune effector cells with a reduction in susceptibility to becoming dysfunctional after multiple or continuous antigen exposures.

### Immune Effector Cell Exhaustion

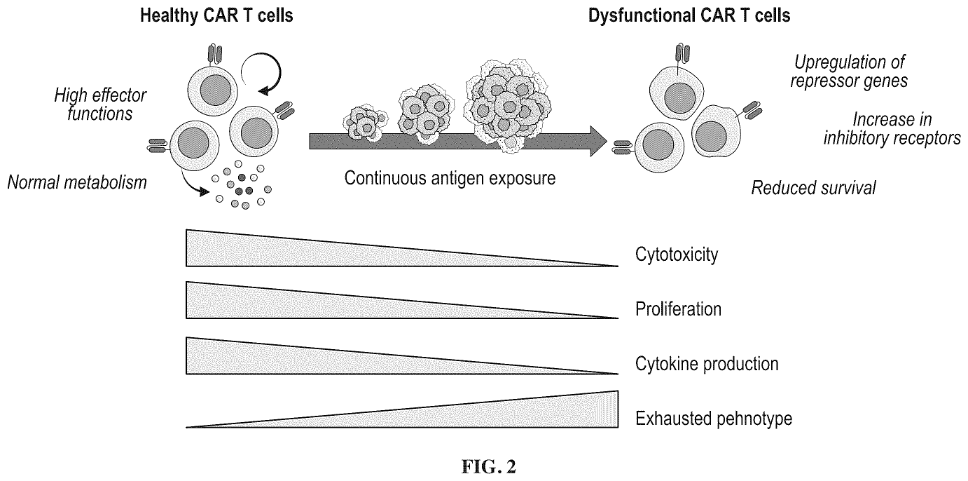

Immune effector cells, such as chimeric antigen receptor (CAR) T cells, are susceptible to becoming dysfunctional or developing an exhausted phenotype when stimulated continuously or multiple times by a target antigen (see FIG. 2). For example, when a CAR-T cells is continuously or repeatedly activated by a target antigen, the CAR-T cell is susceptible to showing a reduction in cytotoxicity, antitumor properties, proliferation, and/or cytokine production as a result of the continuous or repeated activation, and this resulting reduction can be referred to as “dysfunction” an “exhausted phenotype” or “functional exhaustion.” The exhausted phenotype can also be associated with upregulation of repressor genes, increase in inhibitory receptors, reduction in quantity of immune effector cells, and/or reduced survival of the immune effector cells. The exhausted phenotype is epigenetically reinforced. Immune effector cell exhaustion is described further in Jiang, et al., “T-cell exhaustion in the tumor microenvironment,” Cell Death and Disease, 6:e1792 (2015), the disclosure of which is incorporated herein in its entirety by reference for all purposes.

### CAR-T Cell Therapies

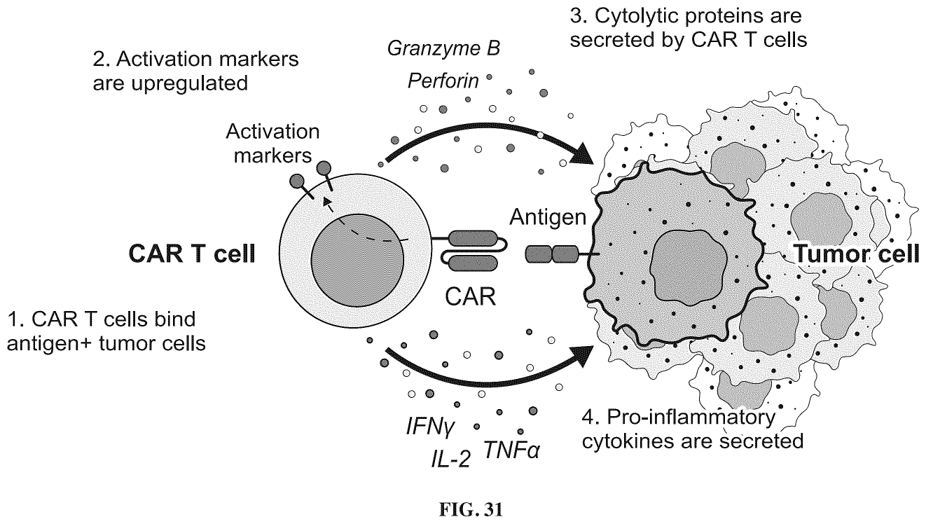

The present disclosure provides immune cells (e.g., T- or NK-cells) modified using nucleobase editors and/or nucleases described herein. The modified immune cells may express chimeric antigen receptors (CARs) (e.g., CAR-T cells). Modification of immune cells to express a chimeric antigen receptor can enhance an immune cell's immunoreactive activity, where the chimeric antigen receptor has an affinity for an epitope on an antigen, and where the antigen is associated with an altered fitness of an organism. For example, the chimeric antigen receptor can have an affinity for an epitope on a protein expressed in a diseased cell. Because the CAR-T cells can act independently of major histocompatibility complex (MHC), activated CAR-T cells can kill the diseased cell expressing the antigen. The direct action of the CAR-T cell evades defensive mechanisms that have evolved in response to MHC presentation of antigens to immune cells. FIG. 31 provides a schematic diagram providing a description of a mechanism of action for CAR-T cells attacking a tumor cell.

The modified immune cells and methods provided herein address known limitations of CAR-T therapy and represent a promising development towards the next generation of precision cell-based therapies.

In embodiments, one or more genes are modified in an immune effector cell so that the cell has a reduced level of, lacks, or have virtually undetectable levels of CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and/or TMEM222. In embodiments, one or more genes are modified in an immune effector cell so that the cell has a reduced level of, lacks, or have virtually undetectable levels of 1, 2, 3, 4, or 5 of the following polypeptides: CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. In embodiments, one or more genes are modified in an immune effector cell so that the cell has a reduced level of, lacks, or have virtually undetectable levels of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC). In embodiments, one or more genes are modified in an immune effector cell so that the cell has a reduced level of, lacks, or have virtually undetectable levels of CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and/or TMEM222 and of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

In embodiments, one or more genes are modified in an immune effector cell so that the cell has a reduced level of, lacks, or have virtually undetectable levels of CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and/or TMEM222, and/or one or more of the following polypeptides relative to an unmodified immune cell: B cell leukemia/lymphoma 11b (Bcl11b); B cell leukemia/lymphoma 2 related protein A1d (Bcl2a1d); B cell leukemia/lymphoma 6 (Bcl6); butyrophilin-like 6 (Btnl6); CD151 antigen (Cd151); chemokine (C—C motif) receptor 7 (Ccr7); discs large MAGUK scaffold protein 5 (Dlg5); erythropoietin (Epo); G protein-coupled receptor 18 (Gpr18); interferon alpha 15 (Ifna15); interleukin 6 signal transducer (Il6st); interleukin 7 receptor (Il7r); Janus kinase 3 (Jak3); membrane associated ring-CH-type finger 7 (Marchf7); NCK associated protein 1 like (Nckap1l); phospholipase A2, group IIF (Pla2g2f); runt related transcription factor 3 (Runx3); Signal-regulatory protein beta 1B (Sirpb1b); transforming growth factor, beta 1 (Tgfb1); tumor necrosis factor (ligand) superfamily, member 14 (Tnfsf14); tumor necrosis factor (ligand) superfamily, member 18 (Tnfsf18); tumor necrosis factor (ligand) superfamily, member 8 (Tnfsf8); zinc finger CCCH type containing 8 (Zc3h8); (Rac family small GTPase 2); (Slc4a1); 5-azacytidine induced gene 2 (Azi2); a disintegrin and metalloprotease domain 17 (Adam 17); a disintegrin and metalloprotease domain 8 (Adam8); Acetyl-CoA Acetyltransferase 1 (ACAT1); ACLY; adapter related protein complex 3 beta 1 sububit (Ap3b1); adapter related protein complex 3 delta 1 sububit (Ap3d1); adenosine A2a receptor (Adora2a); adenosine deaminase (Ada); adenosine kinase (Adk); adenosine regulating molecule 1 (Adrm1); advanced glycosylation end product-specific receptor (Ager) allograft inflammatory factor 1 (Aif1); AKT1; AKT2; amyloid beta (A4) precursor protein-binding family B member 1 interacting protein (Apbb1ip); ankyrin repeat and LEM domain (Ankle1); annecin A1 (Anxa1); arginase liver (Arg 1); arginase type II (Arg 2); AtPase Cu++ transporting, alpha polypeptide (Atp7a); autoimmune regulator (Aire); autophagy related 5 (Atg5); AXL; B and T Lymphocyte Associated (BTLA); B and T lymphocyte associated (Btla); B cell leukemia/lymphoma 10 (Bcl10); B cell leukemia/lymphoma 11a (Bcl11a); B cell leukemia/lymphoma 2 (Bcl2); B cell leukemia/lymphoma 3 (Bcl3); basic leucine zipper transcription factor, ATF-like (Batf); BCL2-associated X protein (Bax); BCL2L11; beta 2 microglobulin (B2m); BL2-associated agonist of cell death (Bad); BLIMP1; Bloom syndrome, RecQ like helicase (Blm); Bmi1 polycomb ring finger oncogene (Bmi1); Bone morphogenic protein 4 (Bmp4); Braf transforming gene (Braf); butyrophilin, subfamily 2, member A1 (Btn2a1); butyrophilin, subfamily 2, member A2 (Btn2a2); butyrophilin-like 1 (Btnl1); butyrophilin-like 2 (Btnl2); c-abl oncogene 1 (Abl1); c-abl oncogene 2 (Abl2); cadherin-like 26(Cdh26); calcium channel, voltage dependent, beta 4 subunit (Cacnb4); CAMK2D; capping protein regulator and myosin 1 linker 2 (Carmil2); carcinoembryonic antigen-related cell adhesion molecule (Ceacam1); Casitas B-lineage lymphoma b (Cblb); CASP8; Caspase 3 (Casp3); caspase recruitment domain family member 11 (Card11); catenin (cadherin associated protein), beta 1 (Ctnnb1); caveolin 1 (Cav1); CBL-B; CCAAT/enhancer binding protein (C/EBP), beta (Cebpb); CCR10; CCR4; CCR5; CCR6; CCR9; CD103; CD11a; CD122; CD123; CD127; CD130; CD132; CD160 antigen (Cd160); CD161; CD19; CD1d1 antigen (Cd1d1); CD1d2 antigen (CD1d2); CD2 antigen (CD2); CD209e antigen (Cd209e); CD23; CD244 molecule A (Cd244a); CD24a antigen (Cd24a); CD27 antigen (CD27); CD274 antigen (Cd274); CD276 antigen (Cd276); CD28 antigen (Cd28); CD3 delta; CD3 epsilon; CD3 gamma; CD30; CD300A molecule (Cd300a); CD33; CD38; CD4 antigen (Cd4); CD40 ligand (Cd40lg); CD44 antigen (Cd44); CD46 antigen, complement regulatory protein (Cd46); CD47 antigen (Rh-related antigen, integrin-associated signal transducer) (Cd47); CD48 antigen (Cd48); CD5 antigen (Cd5); CD52; CD58; CD59b antigen (Cd59b); CD6 antigen (Cd6); CD69; CD7; CD70; CD74 antigen (Cd74); CD8; CD8 antigen (Cd8); CD80 antigen (Cd80); CD81 antigen (Cd81); CD82; CD83 antigen (Cd83); CD86; CD86 antigen (Cd86); CD8A; CD96; CD99; CDK4; CDK8; CDKN1B; chemokine (C motif) ligand 1 (Xcl1); chemokine (C—C motif) ligand 19 (Ccl19); chemokine (C—C motif) ligand 2 (Ccl2); chemokine (C—C motif) ligand 20 (Ccl20); chemokine (C—C motif) ligand 5 (Ccl5); chemokine (C—C motif) receptor 2 (Ccr2); chemokine (C—C motif) receptor 6 (Ccr6); chemokine (C—C motif) receptor 9 (Ccr9); chemokine (C—X—C motif) ligand 12 (Cxcl12); chemokine (C—X—C motif) receptor (Cxcr4); Chitinase 3 Like 1 (Chi3l1); cholinergic receptor, nicotinic, alpha polypeptide 7 (Chrna7); chromodomain helicase DNA binding protein 7 (Chd7); CLA; Class II Major Histocompatibility Complex Transactivator (CIITA); cleft lip and palate associated transmembrane protein 1 (Clptm1); Cluster of Differentiation 123 (CD123); Cluster of Differentiation 3 (CD3); Cluster of Differentiation 33 (CD33); Cluster of Differentiation 52 (CD52); Cluster of Differentiation 7 (CD7); Cluster of Differentiation 96 (CD96); coagulation factor II (thrombin) receptor-like 1 (F2rl1); coil-coil domain containing 88B (Ccdc88b); core-binding factor beta (Cbfb); coronin, actin binding protein 1A (Coro1a); coxsackie virus and adenovirus receptor (Cxadr); CS-1; CSF2CSK; c-src tyrosine kinase (Csk); C-type lectin domain family 2, member i (Clec2i); C-type lectin domain family 4, member a2 (Clec4a2); C-type lectin domain family 4, member d (Clec4d); C-type lectin domain family 4, member e (Clec4e); C-type lectin domain family 4, member f (Clec4f); C-type lectin domain family 4, member g (Clec4g); CUL3; CXCR3; cyclic GMP-AMP synthase (Cgas); cyclin D3 (Ccnd3); cyclin dependent kinase inhibitor 2A (Cdkn2a); cyclin-dependent kinase (Cdk6); CYLD lysine 63 deubiquitinase (Cyld); cysteine-rich protein 3 (Crip3); cytidine 5′-triphosphate synthase (Ctps); Cytochrome P450 Family 11 Subfamily A Member 1 (Cyp11a1); cytochrome P450, family 26, subfamily b, polypeptide (Cyp26b1); Cytokine Inducible SH2 Containing Protein (CISH); cytotoxic T lymphocyte-associated protein 2 alpha (Ctla2a); Cytotoxic T-Lymphocyte Associated Protein 4 (CTLA-4); DCK; dedicator of cytokinesis 2 (Dock2); dedicator of cytokinesis 8 (Dock8); delta like canonical Notch ligand 4 (D114); deltex 1, E3 ubiquitin ligase (Dtx1); deoxyhypusine synthase (Dhps); DGKA; DGKZ; DHX37; dicer 1, ribonuclease type III (Dicer1); dipeptidylpeptidase 4 (Dpp4); discs large MAGUK scaffold protein 1 (Dlg1); DnaJ heat shock protein family (Hsp40) member A3 (Dnaja3); dolichyl-di-phosphooligosaccharide-protein glycotransferase (Ddost); double homeobox B-like 1 (Duxbl1); drosha, ribonuclease type III (Drosha); dual specificity phosphatase 10 (Dusp10); dual specificity phosphatase 22 (Dusp22); dual specificity phosphatase 3 (Dusp3); E74-like factor 4 (Elf4); early growth response 1(Egr1); early growth response 3 (Egr3); ELOB (TCEB2); ENTPD1 (CD39); eomesodermin (Eomes); Eph receptor B4 (Ephb4); Eph receptor B6 (Ephb6); ephrin B1 (Efnb1); ephrin B2 (Efnb2); ephrin B3 (Efnb3); Epstein-Barr virus induced gene 3 (Ebi3); erb-b2 receptor tyrosine kinase (Erbb2); eukaryotic translation initiation factor 2 alpha kinase 4 (Eif2ak4); FADD; family with sequence similarity 49, member B (Fam49b); Fanconi anemia, complementation group A (Fanca); Fanconi anemia, complementation group D2 (Fancd2); Fas (TNF receptor superfamily member 6) (Fas); Fas (TNFRSF6)-associated via death domain (Fadd); Fas Cell Surface Death Receptor (FAS); Fc receptor, IgE, high affinity I, gamma polypeptide (Fcer1g); fibrinogen-like protein 1 (Fgl1); fibrinogen-like protein 2 (Fgl2); FK506 binding protein 1a (Fkbp1a); FK506 binding protein 1b ((Fkbp1b); flotillin 2 (Flot2); FMS-like tyrosine kinase (Flt3); forkhead box J1 (Foxj1); forkhead box N1 (Foxn1); forkhead box P1 (Foxp1); forkhead box P3 (Foxp3); frizzled class receptor 5 (Fzd5); frizzled class receptor 7 (Fzd7); frizzled class receptor 8 (Fzd8); fucosyltransferase 7 (Fut7); Fyn proto-oncogene (Fyn); gap junction protein, alpha 1 (Gja1); GATA binding protein 3 (GATA3); GCN2 kinase (IDO pathway); gelsolin (Gsn); GLI-Kruppel family member GLI3 (Gli3); glycerol-3-phosphate acyltransferase, mitochondrial (Gpam); growth arrest and DNA-damage-inducible 45 gamma (Gadd45g); GTPase, IMAP family member 1 (Gimap1); H1TET2; H2.0-like homeobox (Hlx); haematopoietic 1 (hem1); HCLS1 binding protein 3 (Hs1bp3); heat shock 105 kDa/110 kDa protein 1 (Hsph1); heat shock protein 1 (chaperonin) (Hspd1); heat shock protein 90, alpha (cytosolic), class A member 1 (Hsp90aa1); hematopoietic SH2 domain containing (Hsh2d); hepatitis A virus cellular receptor 2 (Havcr2); hes family bHLH transcription factor 1 (Hes1); histocompatibility 2, class II antigen A, alpha (H2-Aa); histocompatibility 2, class II antigen A, beta 1 (H2-Abl); histocompatibility 2, class II, locus DMa (H2-DMa); histocompatibility 2, M region locus 3 (H3-M3); histocompatibility 2, O region alpha locus (H2-Oa); histocompatibility 2, T region locus 23 (H2-T23); HLA-DR; homeostatic iron regulator (Hfe); icos ligand (Icosl); IKAROS family zinc finger 1 (Ikzf1); IL10; IL10RA; IL2 inducible T cell kinase (Itk); IL6R; Indian hedgehog (Ihh); indoleamine 2,3-dioxygenase 1 (Idol); inducible T cell co-stimulator (Icos); inositol 1,4,5-trisphosphate 3-kinase B (Itpkb); insulin II (Ins2); insulin-like growth factor 1 (Igf1); insulin-like growth factor 2 (Igf2); insulin-like growth factor binding protein 2 (Igfbp2); integrin alpha L (Itgal); integrin alpha M (Itgam); integrin alpha V (Itgav); integrin alpha X (Itgax); integrin beta 2 (Itgb2); integrin, alpha D (Itgad); intercellular adhesion molecule 1 (Icam1); interferon (alpha and beta) receptor 1 (Ifnar1); interferon alpha 1 (Ifna1); interferon alpha 11 (Ifna11); interferon alpha 12 (Ifna12); interferon alpha 13 (Ifna13); interferon alpha 14 (Ifna14); interferon alpha 16 (Ifna16); interferon alpha 2 (Ifna2); interferon alpha 4 (Ifna4); interferon alpha 5 (Ifna5); interferon alpha 6 (Ifna6); interferon alpha 7 (Ifna7); interferon alpha 9 (Ifna9); interferon alpha B (Ifnab); interferon beta 1 (Ifnb1); interferon gamma (IFNg); interferon kappa (Ifnk); interferon regulatory factor 1 (Irf1); interferon regulatory factor 4 (Irf4); interferon zeta (Ifnz); interleukin 1 beta (Il1b; interleukin 1 family, member 8 (Il1f8); interleukin 1 receptor-like 2 (Il1rl2); interleukin 12 receptor, beta1 (Il12rb1); interleukin 12a (Il12a); interleukin 12b (Il12b); interleukin 15 (Il15); interleukin 18 (Il18); interleukin 18 receptor 1 (Il18r1); interleukin 2 (Il2); interleukin 2 receptor, alpha chain (Il2ra); interleukin 2 receptor, gamma chain (Il2rg); interleukin 20 receptor beta (Il20rb); interleukin 21 (Il21); interleukin 23, alpha subunit p19 (Il23a); interleukin 27 (Il27); interleukin 4 (Il4); interleukin 4 receptor, alpha (Il4ra); interleukin 6 (Il6); interleukin 7 (Il7); IRF8; itchy, E3 ubiquitin protein ligase (Itch); jagged 2 (Jag2); jumonji domain containing 6 (Jmjd6); JUNB; junction adhesion molecule like 9 (Jam9); K(lysine) acetyltransferase 2A (Kat2a); KDEL (Lys-Asp-Glu-Leu) endoplasmic reticulum protein retention receptor 1 (Kdelr1); KIT proto-oncogene receptor tyrosine kinase (Kit); LAG-3; LAIR-1 (CD305); LDHA; lectin, galactose binding, soluble 1 (Lgals1); lectin, galactose binding, soluble 3 (Lgals3); lectin, galactose binding, soluble 8 (Lgals8); lectin, galactose binding, soluble 9 (Lgals9); leptin (Lep); leptin receptor (Lepr); leucine rich repeat containing 32 (Lrrc32); leukocyte immunoglobulin-like receptor, subfamily B, member 4A (Lilrb4a); LFNG O-fucosylpeptide 3-beta-N-acetylglucosaminyltransferase (Lfng); LIF; ligase IV, DNA, ATP-dependent (Lig4); LIM domain only 1 (Lmo1); limb region 1 like (Lmbrl); linker for activation of T cells (Lat); lymphocyte antigen 9 (Ly9); lymphocyte cytosolic protein 1 (Lcp1); lymphocyte protein tyrosine kinase (Lck); lymphocyte transmembrane adaptor 1 (Lax1); lymphocyte-activation gene 3 (Lag3); lymphoid enhancer binding factor 1 (Lef1); LYN; lysyl oxidase-like 3 (Loxl3); MAD1 mitotic arrest deficient 1-like 1 (Mad1l1); MALT1 paracaspase (Malt1); MAP4K4; MAPK14; MCJ; mechanistic target of rapamycin kinase (Mtor); MEF2D; Methylation-Controlled J Protein (MCJ); methyltransferase like 3 (Mettl3); MGAT5; MHC I like leukocyte 2 (Mill2); midkine (Mdk); mitogen-activated protein kinase 8 interacting protein 1 (Mapk8ip10); moesin (Msn); myelin protein zero-like 2 (Mpzl2); myeloblastosis oncogene (Myb); myosin, heavy polypeptide 9, non-muscle (Myh9); Nedd4 family interacting protein 1 (Ndfip1); neural precursor cell expressed, developmentally down-regulated 4 (Nedd4); NFATcT; NFATC2; NFATC4; NFKB activating protein (Nkap); nicastrin (Ncstn); NK2 homeobox 3 (Nkx2-3); NLR family, CARD domain containing 3 (Nlrc3); NLR family, pyrin domain containing 3 (Nlrp3); non-catalytic region of tyrosine kinase adaptor protein 1 (Nck1); non-catalytic region of tyrosine kinase adaptor protein 2 (Nck2); non-homologous end joining factor 1 (Nhej1); non-SMC condensin II complex, subunit H2 (Ncaph2); Notch-regulated ankyrin repeat protein (Nrarp); NT5E (CD73); nuclear factor of activated T cells, cytoplasmic, calcineurin dependent (Nfatc3); nuclear factor of kappa light polypeptide gene enhancer in B cells inhibitor, delta (Nfkbid); nuclear receptor co-repressor 1 (Ncor1); ODC1; OTU domain containing 5 (Otud5); OTULINL (FAM105A); paired box 1 (Pax1); PDCD1 (PD1; PD-1); PDIA3; pellino 1 (Peli1); peroxiredoxin 2 (Prdx2); PHD1 (EGLN2); PHD2 (EGLN1); PHD3 (EGLN3); phosphodiesterase 5A, cGMP-specific (Pde5a); phosphoinositide-3-kinase regulatory subunit (Pik3r6); phospholipase A2, group IIA (Pla2g2a); phospholipase A2, group IID (Pla2g2d); phospholipase A2, group IIE (Pla2g2e); phosphoprotein associated with glycosphingolipid microdomains 1 (Pag1); PIK3CD; PIKFYVE; POZ (BTB) and AT hook containing zinc finger 1 (Patz1); PPARa; PPARd; PR domain containing 1, with ZNF domain (Prdm1); presenilin 1 (Psen1); presenilin 2 (Psen2); PRKACA; PRKC, apoptosis, WT1, regulator (Pawr); programmed cell death 1 ligand 2 (Pdcd1lg2); prosaposin (Psap); prostaglandin E receptor 4 (subtype EP4) (Ptger4); protein kinase C, theta 2 (Prkcq); protein kinase C, zeta (Prkcz); protein kinase, cAMP dependent regulatory, type I, alpha (Prkar1a); protein kinase, DNA activated, catalytic polypeptide (Prkdc); protein phosphatase 3, catalytic subunit, beta isoform (Ppp3cb); protein tyrosine phosphatase, non-receptor type 2 (Ptpn2); protein tyrosine phosphatase, non-receptor type 22 (lymphoid) (Ptpn22); protein tyrosine phosphatase, non-receptor type 6 (Ptpn6); protein tyrosine phosphatase, receptor type, C (Ptprc); PTEN; PTPN11; purine-nucleoside phosphorylase (Pnp); purinergic receptor P2X, ligand-gated ion channel, 7 (P2rx7); PVR Related Immunoglobulin Domain Containing (PVRIG; CD112R); PYD and CARD domain containing 7 (Pycard); RAB27A, member RAS oncogene family (Rab27a); RAB29, member RAS oncogene family (Rab29); radical S-adenosyl methionine domain containing 2 (Rsad2); RAR-related orphan receptor alpha (Rora); RAR-related orphan receptor gamma (Ror); RAS guanyl releasing protein 1 (Rasgrp1); ras homolog family member A (Rhoa); ras homolog family member H (Rhoh); RAS protein activator like 3 (Rasal3); RASA2; receptor (TNFRSF)-interacting serine-threonine kinase 2 (Ripk2); recombination activating gene 1 (Rag1); recombination activating gene 2 (Rag2); Regulatory Factor X Associated Ankyrin Containing Protein (RFXANK); RHO family interacting cell polarization regulator 2 (Ripor2); ribosomal protein L22 (Rpl 22); ribosomal protein S6 (Rps6); RING CCCH (C3H) domains 1 (Rc3h1); ring finger and CCCH-type zinc finger domains 2 (Rc3h2); RNF2; runt related transcription factor 1 (Runx1); runt related transcription factor 2 (Runx2); SAM and SH3 domain containing 3 (Sash3); schlafen 1; Selectin P Ligand/P-Selectin Glycoprotein Ligand-1 (SELPG/PSGL1) polypeptide; selenoprotein K (Selenok); sema domain immunoglobulin domain (Ig), transmembrane domain (TM) and short cytoplasmic domain, (semaphorin) 4A (Sema4a); serine/threonine kinase 11 (Stk11); SH3 domain containing ring finger 1 (Sh3rf1); SHP1; sialophorin (Spn); SIGLEC15; signal transducer and activator of transcription 3 (Stat3); signal transducer and activator of transcription 5A (Stat5A); signal transducer and activator of transcription 5B (Stat5B); signal-regulatory protein alpha (Sirpa); Signal-regulatory protein beta 1A (Sirpb1a); Signal-regulatory protein beta 1C (Sirpb1c); SLA; SLAM family member 6 (Slamf6); SLAMF7; SMAD family member 3 (Smad3); SMAD family member 7 (Smad7); SMARCA4; solute carrier family 11 (proton-coupled divalent metal ion transporters), member 1 (Slc11a1); solute carrier family 4 (anion exchanger), member 1; solute carrier family 46, member 2 (Slc46a2); sonic hedgehog (Shh); SOS Ras/Rac guanine nucleotide exchange factor 1 (Sos1); SOS Ras/Rac guanine nucleotide exchange factor 2 (Sos2); special AT-rich sequence binding protein 1 (Satb1); spleen tyrosine kinase (Syk); Sprouty RTK Signaling Antagonist 1 (Spry1); Sprouty RTK Signaling Antagonist 2 (Spry2); squamous cell carcinoma antigen recognized by T cells (Sart1); src homology 2 domain-containing transforming protein B (Shb); Src-like-adaptor 2 (Sla2); SRY (sex determining region Y)-box 4 (Sox4); STK4; suppression inducing transmembrane adaptor 1 (Sit1); suppressor of cytokine signaling 1 (Socs1); suppressor of cytokine signaling 5 (Socs5); suppressor of cytokine signaling 6 (Socs6); surfactant associated protein D (Sftpd); SUV39; syndecan 4 (Sdc4); syntaxin 11 (Stx11); T Cell Immunoglobulin Mucin 3 (Tim-3); T cell immunoreceptor with Ig and ITIM domains (Tigit); T cell receptor alpha joining 18 (Traj18); T Cell Receptor Beta Constant 1 (TRBC1); T Cell Receptor Beta Constant 2 (TRBC2); T cell, immune regulator 1, ATPase, H+ transporting, lysosomal V0 protein A3 (Tcirg1); T cell-interacting, activating receptor on myeloid cells 1 (Tarm1); T-box 21 (Tbx21); TCR; TCR alpha; TCR beta; TCR complex gene sequence; Tet Methylcytosine Dioxygenase 2 (TET2); TGFbRII; TGFbRII (TGFBR2); three prime repair exonuclease 1 (Trex1); thymocyte selection associated (Themis); thymus cell antigen 1, theta (Thy 1); TMEM222; TNF receptor-associated factor 6 (Traf6); TNFAIP3; TNFRSF10B; TNFRSF8 (CD30); TOX; TOX2; TRAC; transformation related protein 53 (Trp53); Transforming Growth Factor Beta Receptor II (TGFbRII); transforming growth factor, beta receptor II (Tgfbr2); transmembrane 131 like (Tmem131l); transmembrane protein 98 (Tmem98); triggering receptor expressed on myeloid cells-like 2 (Treml2); TSC complex subunit 1 (Tsc1); tumor necrosis factor (ligand) superfamily, member 11 (Tnfsf11); tumor necrosis factor (ligand) superfamily, member 13b (Tnfsf13b); tumor necrosis factor (ligand) superfamily, member 4 (Tnfsf4); tumor necrosis factor (ligand) superfamily, member 9 (Tnfsf9); tumor necrosis factor receptor superfamily, member 13c (Tnfrsf13c); tumor necrosis factor receptor superfamily, member 4 (Tnfrsf4); tumor necrosis factor, alpha-induced protein 8-like 2 (Tnfa1p8l2); twisted gastrulation BMP signaling modulator 1 (Twsg1); UBASH3A; vanin 1 (Vnn1); vascular cell adhesion molecule 1 (Vcam1); VHL; v-maf musculoaponeurotic fibrosarcoma oncogene family, protein B (avian) (Mafb); V-set and immunoglobulin domain containing 4 (Vsig4); V-Set Immunoregulatory Receptor (VISTA); WD repeat and FYVE domain containing 4 (Wdfy4); wingless-type MMTV integration site family, member 1 (Wnt1); wingless-type MMTV integration site family, member 4 (Wnt4); WNT signaling pathway regulator (Apc); WW domain containing E3 ubiquitin protein ligase 1 (Wwp1); XBP1; YAP1; ZAP70; ZC3H12A; zfp35; zinc finger and BTB domain containing 1 (Zbtb1); zinc finger and BTB domain containing 7B (Zbtb7B); zinc finger CCCH type containing 12A (Zc3h12a); zinc finger CCCH type containing 12D (Zc3h12d); zinc finger E-box binding homeobox 1 (Zeb1); zinc finger protein 36, C3H type (Zfp36); zinc finger protein 36, C3H type-like 1 (Zfp36L1); zinc finger protein 36, C3H type-like 2 (Zfp36L2); and zinc finger protein 683 (Zfp683).

Immune cells and/or immune effector cells can be isolated or purified from a sample collected from a subject/donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the present disclosure provides T cells that have targeted gene knock-outs at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRαβ T cells are removed using CliniMACS magnetic bead depletion to minimize the risk of GVHD. In another embodiment, the present disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878).

Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

In embodiments, the immune effector cells contemplated in the present disclosure are effector T cells. In some embodiments, the effector T cell is a naïve CD8⁺ T cell, a cytotoxic T cell, a natural killer T (NKT) cell, a natural killer (NK) cell, or a regulatory T (T_{reg}) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor (CAR), an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Provided herein are also polynucleotides that encode the chimeric antigen receptors (CARs) described herein. In some embodiments, the nucleic acid molecule is isolated or purified. Delivery of the nucleic acid molecules ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen receptor (CAR) and an altered endogenous gene that provides resistance to development of an exhausted phenotype after repeated or continuous exposure to an antigen and/or increased persistence, resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in a start codon, an exon, an intron, a splice acceptor site, a splice donor site, an exon-intron injunction, or a regulatory element thereof. The modification may be edit to a single nucleobase in a gene or a regulatory element thereof. The modification may be in a exon, more than one exons, an intron, or more than one introns, or a combination thereof. The modification may be in an open reading frame of a gene. The modification may be in an untranslated region of the gene, for example, a 3′-UTR or a 5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g., a Kozak sequence), or any combination thereof.

Immune effector cells expressing an endogenous immune cell receptor and a chimeric antigen receptor (CAR) may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide resistance to development of an exhausted phenotype after repeated or continuous exposure to an antigen, increased persistence, fratricide resistance, enhance the function of the immune cell or to reduce immunosuppression or inhibition can occur in the immune cell before the cell is transformed to express a chimeric antigen receptor (CAR). In other aspects, editing of genes to provide resistance to development of an exhausted phenotype after repeated or continuous exposure to an antigen, increase persistence, provide fratricide resistance, enhance the function of the immune cell or to reduce immunosuppression or inhibition can occur in a CAR-T cell, i.e., after the immune cell has been transformed to express a chimeric antigen receptor (CAR).

In some embodiments, the immune cell may comprise one or more edited genes, one or more regulatory elements thereof, or combinations thereof, wherein expression of the edited gene is either knocked out or knocked down. In some embodiments, the immune cell may comprise one or more edited genes, one or more regulatory elements thereof, or combinations thereof, wherein expression of the edited gene is increased. In some embodiments, the immune cell may comprise a chimeric antigen receptor (CAR) and one or more edited genes, one or more regulatory elements thereof, or combinations thereof, wherein expression of the edited gene is either knocked out or knocked down. In some embodiments, the immune cell may comprise a chimeric antigen receptor (CAR) and one or more edited genes, one or more regulatory elements thereof, or combinations thereof, wherein expression of the edited gene is increased.

In some embodiments, the CAR-T cells have reduced (e.g., a negative alteration of at least 10%, 25%, 50%, 75%, or 100%) or inactivated surface HLA class-I expression as compared to a similar CAR-T cell lacking one or more edited genes as described herein. In some embodiments, the CAR-T cells have resistance to development of an exhausted phenotype after repeated or continuous exposure to an antigen as compared to a similar CAR-T cell lacking one or more edited genes as described herein. In some embodiments, the CAR-T cells have increased persistence as compared to a similar CAR-T cell lacking one or more edited genes as described herein. In some embodiments, the CAR-T cells have increased fratricide resistance as compared to a similar CAR-T cell lacking one or more edited genes as described herein. In some embodiments, the CAR-T cells have reduced immunogenicity as compared to a similar CAR-T cell lacking one or more edited genes as described herein. In some embodiments, the CAR-T cells have lower activation threshold as compared to a similar CAR-T lacking one or more edited genes as described herein. In some embodiments, the CAR-T cells have increased anti-neoplasia activity as compared to a similar CAR-T cell lacking one or more edited genes as described herein. In some embodiments, the CAR-T cells have increased T- and/or NK-cell resistance as compared to a similar CAR-T cell lacking one or more edited genes as described herein. The one or more genes may be edited by base editing. In some embodiments the one or more genes are directed to components of the peptide loading complex (PLC) or regulatory components thereof. In some embodiments the one or more genes may be selected from a group consisting of: β2M, TAP1, TAP2, Tapasin, and CD58. In some embodiments, the one or more genes may be selected from the group consisting of CBLB, CD5, Chop, CISH, DCK, DGKα/ζ, DHX37, DNMT3A, EIF2A, FLI-1, ID3, IKZF2, IL-6, PFN1, PRDM1, PRKACA, PTP1B, PTPN6, RASA2, Regnase-1, Roquin-1, SOCS1, SOX4, TLE, TMEM184B, and TMEM222. In some embodiments, the one or more genes are selected from the group consisting of B2M, CD3e, CD3g, CIITA, PD1, and TRAC. In embodiments, the gene corresponds to an antigen targeted by a CAR expressed by the cell. In some the genes may be edited by base editing and or using a nuclease (e.g., Cas12b). In some cases, the one or more genes are selected from CD58, CD115, CD48, MICA, MICB, Nectin-2, ULBP, β2M, TAP1, TAP2, TAPBP, PDIA3, NLRC5, HLA-A, HLA-B, and/or HLA-C. In some embodiments, one or more additional genes may be edited using a base editor or nuclease. In some embodiments, the one or more additional genes may be selected from TRAC and CIITA. In some embodiments, the one or more additional genes edited may be selected from HLA-E, HLA-G, PD-L1, and CD47. In some embodiments, one or more of β2M, TAP1, TAP2, Tapasin, and/or CD58 are edited in combination with edits in each of HLA-E, HLA-G, PD-L1, and CD47.

In some embodiments, the one or more genes are selected from CD5, CD7, CD19, B2M, CD3γ, CIITA, CD38, and PD1. In some embodiments, the CAR-T cells contain modifications in genes encoding one or more of CD5, CD7, CD19, B2M, CD3γ, CIITA, CD38, and PD1. In some embodiments, the CAR-T cells have reduced or undetectable expression of one or more of CD5, CD7, CD19, B2M, CD3γ, CIITA, CD38, and PD1 relative to a wild type or unedited T cell.

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g., a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, for example, such as a PD1 gene, a PDC1 gene, or a member related to or regulating the pathway of their formation or activation.

In some embodiments, provided herein is an immune cell with an edited gene in the peptide loading complex (PLC) or a regulatory element thereof, such that the immune cell does not express or expresses at reduced levels surface HLA class-I peptides. In some embodiments, provided herein is an immune cell with an edited gene in the peptide loading complex (PLC) or a regulatory element thereof, such that the immune cell has increased persistence. In some embodiments, the immune cell comprises an edited gene in the peptide loading complex (PLC) or a regulatory element thereof, and additionally, at least one edited gene.

In some embodiments, provided herein is an immune cell (e.g., T- or NK-cell) with an edited β2M gene, such that the immune cell does not express an endogenous functional Beta-2-microglobulin. In some embodiments, provided herein is an immune cell with an edited β2M gene, such that the immune cell does not express or expresses at reduced levels surface HLA class-I peptides. In some embodiments, provided herein is an immune cell with an edited β2M gene, such that the immune cell has increased persistence. In some embodiments, the immune cell comprises an edited β2M gene, and additionally, at least one edited gene.

In some embodiments, provided herein is an immune cell (e.g., T- or NK-cell) with an edited TAP1 gene, such that the immune cell does not express an endogenous functional TAP1. In some embodiments, provided herein is an immune cell with an edited TAP1 gene, such that the immune cell does not express or expresses at reduced levels surface HLA class-I peptides. In some embodiments, provided herein is an immune cell with an edited TAP1 gene, such that the immune cell has increased persistence. In some embodiments, the immune cell comprises an edited TAP1 gene, and additionally, at least one edited gene.

In some embodiments, provided herein is an immune cell (e.g., T- or NK-cell) with an edited TAP2 gene, such that the immune cell does not express an endogenous functional TAP2. In some embodiments, provided herein is an immune cell with an edited TAP2 gene, such that the immune cell does not express or expresses at reduced levels surface HLA class-I peptides. In some embodiments, provided herein is an immune cell with an edited TAP2 gene, such that the immune cell has increased persistence. In some embodiments, the immune cell comprises an edited TAP2 gene, and additionally, at least one edited gene.

In some embodiments, provided herein is an immune cell (e.g., T- or NK-cell) with edited TAP1 and TAP2 genes, such that the immune cell does not express endogenous functional TAP1 and TAP2. In some embodiments, provided herein is an immune cell with edited TAP1 and TAP2 genes, such that the immune cell does not express or expresses at reduced levels surface HLA class-I peptides. In some embodiments, provided herein is an immune cell with an edited TAP1 and TAP2 gene, such that the immune cell has increased persistence. In some embodiments, the immune cell comprises an edited TAP1 and TAP2 gene, and additionally, at least one edited gene.

In some embodiments, provided herein is an immune cell (e.g., T- or NK-cell) with an edited Tapasin gene, such that the immune cell does not express an endogenous functional Tapasin. In some embodiments, provided herein is an immune cell with an edited Tapasin gene, such that the immune cell does not express or expresses at reduced levels surface HLA class-I peptides. In some embodiments, provided herein is an immune cell with an edited Tapasin gene, such that the immune cell has increased persistence. In some embodiments, the immune cell comprises an edited Tapasin gene, and additionally, at least one edited gene.

In some embodiments, provided herein is an immune cell (e.g., T- or NK-cell) with an edited CD58 gene, such that the immune cell does not express an endogenous functional CD58. In some embodiments, provided herein is an immune cell with an edited CD58 gene, such that the immune cell has increased persistence. In some embodiments, the immune cell comprises an edited CD58 gene, and additionally, at least one edited gene.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Chimeric Antigen Receptors and CAR-T Cells

The disclosure provides immune cells modified using nucleobase editors described herein and that express chimeric antigen receptors (CARs). Modification of immune cells to express a chimeric antigen receptor can enhance an immune cell's immunoreactive activity, wherein the chimeric antigen receptor has an affinity for an epitope on an antigen, wherein the antigen is associated with an altered fitness of an organism. For example, the chimeric antigen receptor can have an affinity for an epitope on a protein expressed in a neoplastic cell. Because the CAR-T cells can act independently of major histocompatibility complex (MHC), activated CAR-T cells can kill the neoplastic cell expressing the antigen. The direct action of the CAR-T cell evades neoplastic cell defensive mechanisms that have evolved in response to MHC presentation of antigens to immune cells. Exemplary chimeric antigen receptors, modified immune cells, and methods for preparing the same are described in PCT Applications No. PCT/US2020/013964, PCT/US2020/052822, PCT/US2020/018178, PCT/US2021/52035, and PCT/US2022/075021, or in Hardke-Wolenski, et al., Biomedicines 10:1493 (2022), the disclosures of which are incorporated herein by reference in their entirety for all purposes.

However, target antigens associated with neoplastic cells may also be expressed on healthy immune cells. Accordingly, activated CAR-T cells not only kill neoplastic cells expressing the target antigen but also healthy immune cells that also express the target antigen. To prevent this fratricide or self-killing of immune cells, the disclosure provides a CAR-T that has been modified using nucleobase editors to reduce or eliminate the expression of a target antigen (e.g., CD19) to provide fratricide resistance. In some embodiments, the disclosure provides a fratricide resistant modified immune effector cell that expresses a chimeric antigen receptor to target a neoplastic cell.

Some embodiments comprise autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a disease, such as a neoplasia (e.g., T- or NK-cell malignancy) or autoimmune disease. In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

In another embodiment, the disclosure provides T cells that have targeted gene knockouts at the TCR constant region (TRAC), which is responsible for TCRαβ surface expression. TCRαβ-deficient CAR-T cells are compatible with allogeneic immunotherapy (Qasim et al., Sci. Transl. Med. 9, eaaj2013 (2017); Valton et al., Mol Ther. 2015 September; 23(9): 1507-1518). If desired, residual TCRαβ T cells are removed using CliniMACS magnetic bead depletion to minimize the risk of GVHD. In another embodiment, the disclosure provides donor T cells selected ex vivo to recognize minor histocompatibility antigens expressed on recipient hematopoietic cells, thereby minimizing the risk of graft-versus-host disease (GVHD), which is the main cause of morbidity and mortality after transplantation (Warren et al., Blood 2010; 115(19):3869-3878). Another technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the disclosure include effector T cells. In some embodiments, the effector T cell is a naïve CD8⁺ T cell, a cytotoxic T cell, a natural killer T (NKT) cell, a natural killer (NK) cell, or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, immune effector cells are effector NK cells. In some embodiments, the immune effector cell is any other subset of T cells. The modified immune effector cell may express, in addition to the chimeric antigen receptor, an exogenous cytokine, a different chimeric receptor, or any other agent that would enhance immune effector cell signaling or function. For example, co-expression of the chimeric antigen receptor and a cytokine may enhance the CAR-T cell's ability to lyse a target cell.

Chimeric antigen receptors as contemplated in the present disclosure comprise an extracellular binding domain, a transmembrane domain, and an intracellular domain. Binding of an antigen to the extracellular binding domain can activate the CAR-T cell and generate an effector response, which includes CAR-T cell proliferation, cytokine production, and other processes that lead to the death, inactivation, and/or neutralization of the antigen expressing cell. In some embodiments of the present disclosure, the chimeric antigen receptor further comprises a linker. In some embodiments, the linker is a (GGGGS)ₙ linker (SEQ ID NO: 274). In some embodiments, the linker is a (GGGGS)₃ linker (SEQ ID NO: 679). In some embodiments, a CAR of the present disclosure includes a leader peptide sequence (e.g., N-terminal to the antigen binding domain). An exemplary leader peptide amino acid sequence is: METDTLLLWVLLLWVPGSTG (SEQ ID NO: 680).

In various embodiments, the CAR-T specifically targets a cluster of differentiation 19 (CD19) polypeptide. In some embodiments, the CAR-T specifically targets CD5, CD7, CD19, CD20, CD22, CD79B, or ROR1.

Provided herein are also nucleic acids that encode the chimeric antigen receptors described herein. In some embodiments, the nucleic acid is isolated or purified. Delivery of the nucleic acids ex vivo can be accomplished using methods known in the art. For example, immune cells obtained from a subject may be transformed with a nucleic acid vector encoding the chimeric antigen receptor. The vector may then be used to transform recipient immune cells so that these cells will then express the chimeric antigen receptor. Efficient means of transforming immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

Some aspects of the present disclosure provide for immune cells comprising a chimeric antigen and an altered endogenous gene that provides a reduced tendency relative to unedited CAR-T cells to develop an exhausted phenotype after being stimulated by multiple antigen exposures or continuous exposure to an antigen, resistance to fratricide, enhances immune cell function, resistance to immunosuppression or inhibition, or a combination thereof. In some embodiments, the altered endogenous gene may be created by base editing. In some embodiments, the base editing may reduce or attenuate the gene expression. In some embodiments, the base editing may reduce or attenuate the gene activation. In some embodiments, the base editing may reduce or attenuate the functionality of the gene product. In some other embodiments, the base editing may activate or enhance the gene expression. In some embodiments, the base editing may increase the functionality of the gene product. In some embodiments, the altered endogenous gene may be modified or edited in an exon, an intron, an exon-intron injunction, or a regulatory element thereof. The modification may be edit to a single nucleobase in a gene or a regulatory element thereof. The modification may be in a exon, more than one exons, a start codon, a splice acceptor site, a splice donor site, an intron, or more than one introns, or a combination thereof. The modification may be in an open reading frame of a gene. The modification may be in an untranslated region of the gene, for example, a 3′-UTR or a 5′5′-UTR. In some embodiments, the modification is in a regulatory element of an endogenous gene. In some embodiments, the modification is in a promoter, an enhancer, an operator, a silencer, an insulator, a terminator, a transcription initiation sequence, a translation initiation sequence (e.g., a Kozak sequence), or any combination thereof.

Allogeneic immune cells expressing an endogenous immune cell receptor as well as a chimeric antigen receptor may recognize and attack host cells, a circumstance termed graft versus host disease (GVHD). The alpha component of the immune cell receptor complex is encoded by the TRAC gene, and in some embodiments, this gene is edited such that the alpha subunit of the TCR complex is nonfunctional or absent. Because this subunit is necessary for endogenous immune cell signaling, editing this gene can reduce the risk of graft versus host disease caused by allogeneic immune cells.

In some embodiments, editing of genes to provide a reduced tendency relative to unedited CAR-T cells to develop an exhausted phenotype after being stimulated by multiple antigen exposures or continuous exposure to an antigen, fratricide resistance, enhance the function of the immune cell or to reduce immunosuppression or inhibition can occur in the immune cell before the cell is transformed to express a chimeric antigen receptor. In other aspects, editing of genes to provide a reduced tendency relative to unedited CAR-T cells to develop an exhausted phenotype after being stimulated by multiple antigen exposures or continuous exposure to an antigen, fratricide resistance, enhance the function of the immune cell or to reduce immunosuppression or inhibition can occur in a CAR-T cell, i.e., after the immune cell has been transformed to express a chimeric antigen receptor.

In some embodiments, the immune cell may comprise a chimeric antigen receptor (CAR) and one or more edited genes (e.g., those genes listed herein), one or more regulatory elements thereof, or combinations thereof, wherein expression of the edited gene is either knocked out or knocked down. In some embodiments, the CAR-T cells have a reduced tendency to develop an exhausted phenotype after being stimulated by multiple antigen exposures or continuous exposure to an antigen as compared to a similar reference CAR-T cell not having the one or more edited genes as described herein. In some embodiments, the CAR-T cells have increased fratricide resistance as compared to a similar reference CAR-T cell not having the one or more edited genes as described herein. In some embodiments, the CAR-T cells have reduced immunogenicity as compared to a similar CAR-T cell but without further having the one or more edited genes as described herein. In some embodiments, the CAR-T cells have lower activation threshold as compared to a similar reference CAR-T not having the one or more edited genes as described herein. In some embodiments, the CAR-T cells have increased anti-neoplasia activity as compared to a similar reference CAR-T cell not having the one or more edited genes as described herein. The one or more genes may be edited by base editing.

In some embodiments, an immune cell comprises a chimeric antigen receptor and one or more edited genes, a regulatory element thereof, or combinations thereof. An edited gene may be an immune response regulation gene, an immunogenic gene, a checkpoint inhibitor gene, a gene involved in immune responses, a cell surface marker, e.g., a T cell surface marker, or any combination thereof. In some embodiments, an immune cell comprises a chimeric antigen receptor and an edited gene that is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene, such as a PD-1 gene, or a member related to or regulating the pathway of their formation or activation.

In some embodiments, provided herein is an immune cell with an edited gene (e.g., CD5, CD7, CD19, CD3e, CD3g, B2M, and/or CIITa), such that the immune cell does not express an endogenous functional polypeptide encoded by the gene. In some embodiments, provided herein is a CAR-T cell with an edited gene, such that the CAR-T cell exhibits reduced or negligible expression or no expression of endogenous polypeptide encoded by the gene. In embodiments, the gene encodes CD5, CD7, CD19, CD3e, CD3g, B2M, and/or CIITa.

In some embodiments, each edited gene may comprise a single base edit. In some embodiments, each edited gene may comprise multiple base edits at different regions of the gene. In some embodiments, a single modification event (such as electroporation), may introduce one or more gene edits. In some embodiments at least one, two, three, four, five, six, seven, eight, nine, ten, eleven, twelve, thirteen, fourteen, fifteen, sixteen, seventeen, eighteen, nineteen, twenty or more edits may be introduced in one or more genes simultaneously. In some embodiments, an immune cell, including but not limited to any immune cell comprising an edited gene selected from any of the aforementioned gene edits, can be edited to generate mutations in other genes that enhance the CAR-T's function or reduce immunosuppression or inhibition of the cell.

### Extracellular Binding Domain

The chimeric antigen receptors of the disclosure include an extracellular binding domain. The extracellular binding domain of a chimeric antigen receptor contemplated herein comprises an amino acid sequence of an antibody, or an antigen binding fragment thereof, that has an affinity for a specific antigen. In some embodiments, the antigen is a cluster of differentiation 19 (CD19) polypeptide, or a fragment thereof.

In some embodiments the chimeric antigen receptor comprises an amino acid sequence of an antibody. In some embodiments, the chimeric antigen receptor comprises the amino acid sequence of an antigen binding fragment of an antibody. The antibody (or fragment thereof) portion of the extracellular binding domain recognizes and binds to an epitope of an antigen. In some embodiments, the antibody fragment portion of a chimeric antigen receptor is a single chain variable fragment (scFv). An scFv comprises the light and variable fragments of a monoclonal antibody. In other embodiments, the antibody fragment portion of a chimeric antigen receptor is a multichain variable fragment, which comprises more than one extracellular binding domains and therefore bind to more than one antigen simultaneously. In a multiple chain variable fragment embodiment, a hinge region may separate the different variable fragments, providing necessary spatial arrangement and flexibility.

In some embodiments, the extracellular binding domain is an anti-CD19 scFv. In some cases, the extracellular binding domain is an anti-CD5, anti-CD7, anti-CD19, anti-CD20, anti-CD22, anti-CD79B, or anti-ROR1 scFv.

In other embodiments, the antibody portion of a chimeric antigen receptor comprises at least one heavy chain and at least one light chain. In some embodiments, the antibody portion of a chimeric antigen receptor comprises two heavy chains, joined by disulfide bridges and two light chains, wherein the light chains are each joined to one of the heavy chains by disulfide bridges. In some embodiments, the light chain comprises a constant region and a variable region. Complementarity determining regions residing in the variable region of an antibody are responsible for the antibody's affinity for a particular antigen. Thus, antibodies that recognize different antigens comprise different complementarity determining regions. Complementarity determining regions reside in the variable domains of the extracellular binding domain, and variable domains (i.e., the variable heavy and variable light) can be linked with a linker or, in some embodiments, with disulfide bridges. In some embodiments, the variable heavy chain and variable light chain are linked by a (GGGGS)ₙ linker (SEQ ID NO: 274), wherein the n is an integer from 1 to 10. In some embodiments, the linker is a (GGGGS)₃ linker (SEQ ID NO: 679).

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell.

For example, in some embodiments, the antigen is expressed in a neoplastic cell. In some embodiments, the neoplastic cell is a malignant T-, B-, or NK-cell. In some embodiments, the malignant T-, B-, or NK-cell is a malignant precursor T-, B-, or NK-cell. In some embodiments, the malignant T-, B-, or NK-cell is a malignant mature T-, B-, or NK-cell. Nonlimiting examples of neoplasia include B cell lymphoma, mantle cell lymphoma, T-cell acute lymphoblastic leukemia (T-ALL), mycosis fungoides (MF), Sézary syndrome (SS), Peripheral T/NK-cell lymphoma, Anaplastic large cell lymphoma ALK+, Primary cutaneous T-cell lymphoma, T-cell large granular lymphocytic leukemia, Angioimmunoblastic T/NK-cell lymphoma, Hepatosplenic T-cell lymphoma, Primary cutaneous CD30+lymphoproliferative disorders, Extranodal NK/T-cell lymphoma, Adult T-cell leukemia/lymphoma, T-cell prolymphocytic leukemia, Subcutaneous panniculitis-like T-cell lymphoma, Primary cutaneous gamma-delta T-cell lymphoma, Aggressive NK-cell leukemia, and Enteropathy-associated T-cell lymphoma.

Antibody-antigen interactions are noncovalent interactions resulting from hydrogen bonding, electrostatic or hydrophobic interactions, or from van der Waals forces. The affinity of extracellular binding domain of the chimeric antigen receptor for an antigen can be calculated with the following formula:

$${K}_{A} = [ Antibody - Antigen ] / [ Antibody ] [ Antigen ] , wherein$$

$$[ Ab ] = molar concentration of unoccupied binding sites on the antibody ;$$

$$[ Ag ] = molar concentration of unoccupied binding sites on the antigen ; \\ and$$

$$[ Ab - Ag ] = molar concentration of the antibody - antigen complex .$$

The antibody-antigen interaction can also be characterized based on the dissociation of the antigen from the antibody. The dissociation constant (K_{D}) is the ratio of the association rate to the dissociation rate and is inversely proportional to the affinity constant. Thus, K_{D}=1/K_{A}. Those skilled in the art will be familiar with these concepts and will know that traditional methods, such as ELISA assays, can be used to calculate these constants.

### Transmembrane Domain

The chimeric antigen receptors of the disclosure include a transmembrane domain. The transmembrane domain of the chimeric antigen receptors described herein spans the CAR-T cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell. Examples of such proteins include, but are not limited to, subunits of the T cell receptor (TCR) complex, PD1, or any of the Cluster of Differentiation proteins, or other proteins, that are expressed in the immune effector cell and that have a transmembrane domain. In some embodiments, the transmembrane domain will be synthetic, and such sequences will comprise many hydrophobic residues.

Transmembrane domains for use in the disclosed CARs can include at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, the transmembrane domain is derived from CD4, CD8α, CD28 and CD3ζ.

The chimeric antigen receptor is designed, in some embodiments, to comprise a spacer between the transmembrane domain and the extracellular domain, the intracellular domain, or both. Such spacers can be 1, 2, 3, 4, 5, 6, 7, 8, 9, 10, 11, 12, 13, 14, 15, 16, 17, 18, 19, or 20 amino acids in length. In some embodiments, the spacer can be 20, 30, 40, 50, 60, 70, 80, 90, or 100 amino acids in length. In still other embodiments the spacer can be between 100 and 500 amino acids in length. The spacer can be any polypeptide that links one domain to another and are used to position such linked domains to enhance or optimize chimeric antigen receptor function.

### Intracellular Signaling Domain

The chimeric antigen receptors of the disclosure include an intracellular signaling domain. The intracellular signaling domain is the intracellular portion of a protein expressed in a T cell that transduces a T cell effector function signal (e.g., an activation signal) and directs the T cell to perform a specialized function. T cell activation can be induced by a number of factors, including binding of cognate antigen to the T cell receptor on the surface of T cells and binding of cognate ligand to costimulatory molecules on the surface of the T cell. A T cell co-stimulatory molecule is a cognate binding partner on a T cell that specifically binds with a co-stimulatory ligand, thereby mediating a co-stimulatory response by the T cell, such as, but not limited to, proliferation. Co-stimulatory molecules include but are not limited to an MHC class I molecule. Activation of a T cell leads to immune response, Such as T cell proliferation and differentiation (see, e.g., Smith-Garvin et al., Annu. Rev. Immunol., 27:591-619, 2009). Exemplary T cell signaling domains are known in the art. Non-limiting examples include the CD3ζ, CD8, CD28, CD27, CD154, GITR (TNFRSF18), CD134 (OX40), and CD137 (4-1BB) signaling domains.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

In some embodiments, the primary signaling domain comprises one or more immunoreceptor tyrosine-based activation motifs, or ITAMs. In some embodiments, the primary signaling domain comprises more than one ITAM. ITAMs incorporated into the chimeric antigen receptor may be derived from ITAMs from other cellular receptors. In some embodiments, the primary signaling domain comprising an ITAM may be derived from subunits of the TCR complex, such as CD3γ, CD3ε, CD3ζ, or CD3δ. In some embodiments, the primary signaling domain comprising an ITAM may be derived from FcRγ, FcRβ, CD5, CD22, CD79a, CD79b, or CD66d.

In some embodiments, the primary signaling domain is selected from the group consisting of CD8, CD28, CD134 (OX40), CD137 (4-1BB), and CD3ζ.

In some embodiments, the secondary, or co-stimulatory, signaling domain is derived from CD2, CD4, CDS, CD8α, CD28, CD83, CD134, CD137 (4-1BB), ICOS, or CD154, or a combination thereof. In some embodiments, the co-signaling domain is a cytoplasmic domain.

In some embodiments, the CAR comprises one or more signaling domains. In some embodiments, the CAR comprises a combination of signaling domains.

### Editing of Target Genes in Immune Cells

In some embodiments, provided herein is an immune cell with at least one modification in an endogenous gene or regulatory elements thereof. In some embodiments, the immune cell may comprise a further modification in at least one, two, three, four, five, six, seven, eight, nine, ten, eleven, twelve, thirteen, fourteen, fifteen, sixteen, seventeen, eighteen, nineteen, twenty or more endogenous genes or regulatory elements thereof. In some embodiments, the at least one modification is a single nucleobase modification. In some embodiments, the at least one modification is by base editing. The base editing may be positioned at any suitable position of the gene, or in a regulatory element of the gene. Thus, it may be appreciated that a single base editing at a start codon, for example, can completely abolish the expression of the gene. In some embodiments, the base editing may be performed at a site within an exon. In some embodiments, the base editing may be performed at a site on more than one exons. In some embodiments, the base editing may be performed at a start codon. In some embodiments, the base editing may be performed at a splice acceptor site. In some embodiments, the base editing may be performed at a splice donor site. In some embodiments, the base editing may be performed at any exon of the multiple exons in a gene. In some embodiments, base editing may introduce a premature STOP codon into an exon, resulting in either lack of a translated product or in a truncated that may be misfolded and thereby eliminated by degradation, or may produce an unstable mRNA that is readily degraded. In some embodiments, the immune cell is a T cell. In some embodiments, the immune cell is a CAR-T cell. In some embodiments, the immune cell is a NK cell.

In some embodiments, the immune cell is modified using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes). In some embodiments, the cell is modified using a CRISPR/Cas system. In some cases, expression of a gene in the cell may be disrupted through introduction of an insertion/deletion (indel) mutation to the gene using, e.g., a nuclease, such as a Cas12b or Cas9 protein, or through insertion of a heterologous polynucleotide sequence into the gene, such as through the use of a transposon or a CRISPR/Cas system.

In some embodiments, an edited gene may be an immune response regulation gene, an immunogenic gene, a checkpoint inhibitor gene, a gene involved in immune responses, a cell surface marker, e.g., a T cell surface marker, or any combination thereof. In some embodiments, the edited gene is associated with activated T cell proliferation, alpha-beta T cell activation, gamma-delta T cell activation, positive regulation of T cell proliferation, negative regulation of T-helper cell proliferation or differentiation, or their regulatory elements thereof, or combinations thereof. In some embodiments, the edited gene may be a checkpoint inhibitor gene.

In some embodiments, the gene is selected from those genes listed herein. Further non-limiting examples of genes that may be edited include those listed in any one of PCT Applications No. PCT/US2020/013964, PCT/US2020/052822, PCT/US2020/018178, PCT/US2021/52035, and PCT/US2022/075021, the disclosures of which are incorporated herein by reference in their entirety for all purposes.

In some embodiments, the editing of the endogenous gene reduces expression of the gene. In some embodiments, the editing of the endogenous gene reduces expression of the gene by at least 50% as compared to a control cell without the modification. In some embodiments, the editing of the endogenous gene reduces expression of the gene by at least 60% as compared to a control cell without the modification. In some embodiments, the editing of the endogenous gene reduces expression of the gene by at least 70% as compared to a control cell without the modification. In some embodiments, the editing of the endogenous gene reduces expression of the gene by at least 80% as compared to a control cell without the modification. In some embodiments, the editing of the endogenous gene reduces expression of the gene by at least 90% as compared to a control cell without the modification. In some embodiments, the editing of the endogenous gene reduces expression of the gene by at least 100% as compared to a control cell without the modification. In some embodiments, the editing of the endogenous gene eliminates gene expression.

In some embodiments, base editing may be performed on an intron. For example, base editing may be performed on an intron. In some embodiments, the base editing may be performed at a site within an intron. In some embodiments, the base editing may be performed at a site one or more introns. In some embodiments, the base editing may be performed at any exon of the multiple introns in a gene. In some embodiments, one or more base editing may be performed on an exon, an intron or any combination of exons and introns.

In some embodiments, the modification or base edit may be within a promoter site. In some embodiments, the base edit may be introduced within an alternative promoter site. In some embodiments, the base edit may be in a 5′ regulatory element, such as an enhancer. In some embodiment, base editing may be introduced to disrupt the binding site of a nucleic acid binding protein. Exemplary nucleic acid binding proteins may be a polymerase, nuclease, gyrase, topoisomerase, methylase or methyl transferase, transcription factors, enhancer, PABP, zinc finger proteins, among many others.

In some embodiments, base editing may be used for splice disruption to silence target protein expression. In some embodiments, base editing may generate a splice acceptor-splice donor (SA-SD) site. Targeted base editing generating a SA-SD, or at a SA-SD site can result in reduced expression of a gene. In some embodiments, base editors (e.g., ABE, CBE, CABE) are used to target dinucleotide motifs that constitute splice acceptor and splice donor sites, which are the first and last two nucleotides of each intron. In some embodiments, splice disruption is achieved with an adenosine base editor (ABE). In some embodiments, splice disruption is achieved with a cytidine base editor (CBE). In some embodiments, base editors (e.g., CBE, CABE) are used to edit exons by creating STOP codons.

In some embodiments, provided herein is an immune cell with at least one modification in one or more endogenous genes. In some embodiments, the immune cell may have at least one modification in one, two, three, four, five, six, seven, eight, nine, ten, eleven, twelve, thirteen, fourteen, fifteen, sixteen, seventeen, eighteen, nineteen, twenty or more endogenous genes. In some embodiments, the modification generates a premature stop codon in the endogenous genes. In some embodiments, the STOP codon silences target protein expression. In some embodiments, the modification is a single base modification. In some embodiments, the modification is generated by base editing. The premature stop codon may be generated in an exon, an intron, or an untranslated region. In some embodiments, base editing may be used to introduce more than one STOP codon, in one or more alternative reading frames. In some embodiments, the stop codon is generated by a adenosine base editor (ABE). In some embodiments, the stop codon is generated by a cytidine base editor (CBE). In some embodiments, the CBE generates any one of the following edits (shown in underlined font) to generate a STOP codon: CAG→TAG; CAA→TAA; CGA→TGA; TGG→TGA; TGG→TAG; or TGG→TAA.

In some embodiments, modification/base edits may be introduced at a 3′-UTR, for example, in a poly adenylation (poly-A) site. In some embodiments, base editing may be performed on a 5′-UTR region.

### Editing of Target Genes

To produce the gene edits described herein using base editing, cells are contacted with one or more guide RNAs and a nucleobase editor polypeptide comprising a nucleic acid programmable DNA binding protein (napDNAbp) and a cytidine deaminase or adenosine deaminase or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid, where the at least one nucleic acid encodes one or more guide RNAs and a nucleobase editor polypeptide containing a nucleic acid programmable DNA binding protein (napDNAbp) and a deaminase. In some embodiments, the gRNA comprises nucleotide analogs. In some instances, the gRNA is added directly to a cell. These nucleotide analogs can inhibit degradation of the gRNA from cellular processes. Tables 1 and 2A-2C provide representative sequences to be used for gRNAs. In some embodiments, the gene edits described herein are introduced to a polynucleotide using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes). In some embodiments, the gene edits described herein are introduced to a polynucleotide using a CRISPR/Cas system. In some cases, expression of a gene may be disrupted through introduction of an insertion/deletion (indel) mutation to the gene using, e.g., a nuclease, such as a Cas12b or Cas9 protein, or through insertion of a heterologous polynucleotide sequence into the gene, such as through the use of a transposon or a CRISPR/Cas system.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In embodiments, a guide RNA of the disclosure contains a scaffold. A non-limiting example of a scaffold nucleotide sequence is the following: GUUUUAGAGCUAGAAAUAGCAAGUUAAAAUAAGGCUAGUCCGUUAUCAACUUGAAAAAGUGG CACCGAGUCGGUGCU*mU*mU*mU (SEQ ID NO: 1452), where the symbol “*mU” indicates a uracil nucleotide with a 2′-O-methyl modification and linked to the listed 5′ nucleotide by a 3′-phosphorothioate.

Exemplary guide RNA sequences are provided in Tables 1, and 2A-2C below.

TABLE 1
Exemplary guide polynucleotide sequences.

| Guide | Alternative | | SEQ ID |
|---|---|---|---|
| Name | Name | sgRNA Sequence¹ | NO |
| EF8 | LP | AUGUACCUGGUCCAAGGAACGUUUUAGAGCUAGAAAUAG | 427 |
| | sgRNA26 | CAAGUUAAAAUAAGGCUAGUCCGUUAUCAACUUGAAAAA | |
| | | GUGGCACCGAGUCGGUGCU*mU*mU*mU | |
| EF7 | LP | UAGGAUGGUAGCACACAACCGUUUUAGAGCUAGAAAUAG | 426 |
| | sgRNA19 | CAAGUUAAAAUAAGGCUAGUCCGUUAUCAACUUGAAAAA | |
| | | GUGGCACCGAGUCGGUGCU*mU*mU*mU | |
| EF9 | LP | GGUUCAUACCUUGAAACAGGGUUUUAGAGCUAGAAAUAG | 428 |
| | sgRNA27 | CAAGUUAAAAUAAGGCUAGUCCGUUAUCAACUUGAAAAA | |
| | | GUGGCACCGAGUCGGUGCU*mU*mU*mU | |
| EF10 | LP | AGGUCCUACCUUGUCAAUAAGUUUUAGAGCUAGAAAUAG | 429 |
| | sgRNA28 | CAAGUUAAAAUAAGGCUAGUCCGUUAUCAACUUGAAAAA | |
| | | GUGGCACCGAGUCGGUGCU*mU*mU*mU | |
| EF11 | Effi-sg17 | UUUAGGAACCUCCUUAUAGCGUUUUAGAGCUAGAAAUAG | 430 |
| | | CAAGUUAAAAUAAGGCUAGUCCGUUAUCAACUUGAAAAA | |
| | | GUGGCACCGAGUCGGUGCU*mU*mU*mU | |
| EF12 | Effi-sg18 | GCUUACUUCUUCCGGAUGAAGUUUUAGAGCUAGAAAUAG | 431 |
| | | CAAGUUAAAAUAAGGCUAGUCCGUUAUCAACUUGAAAAA | |
| | | GUGGCACCGAGUCGGUGCU*mU*mU*mU | |
| EF13 | LP | CCUGCAGGACGGAGAGGAGCGUUUUAGAGCUAGAAAUAG | 432 |
| | sgRNA35 | CAAGUUAAAAUAAGGCUAGUCCGUUAUCAACUUGAAAAA | |
| | | GUGGCACCGAGUCGGUGCU*mU*mU*mU | |
| EF14 | Effi-sg19 | GCGGGCAUCUGGGCGCCGGGGUUUUAGAGCUAGAAAUAG | 433 |
| | | CAAGUUAAAAUAAGGCUAGUCCGUUAUCAACUUGAAAAA | |
| | | GUGGCACCGAGUCGGUGCU*mU*mU*mU | |
| EF15 | Effi-sg20 | GGACUCACCCGCUUCUGCAGGUUUUAGAGCUAGAAAUAG | 434 |
| | | CAAGUUAAAAUAAGGCUAGUCCGUUAUCAACUUGAAAAA | |
| | | GUGGCACCGAGUCGGUGCU*mU*mU*mU | |
| EF16 | Effi-sg21 | CUCCACAGGCAAAGAACAGAGUUUUAGAGCUAGAAAUAG | 435 |
| | | CAAGUUAAAAUAAGGCUAGUCCGUUAUCAACUUGAAAAA | |
| | | GUGGCACCGAGUCGGUGCU*mU*mU*mU | |
| EF17 | Effi-sg22 | CUCCAGGACGGCCGGGGCUUGUUUUAGAGCUAGAAAUAG | 436 |
| | | CAAGUUAAAAUAAGGCUAGUCCGUUAUCAACUUGAAAAA | |
| | | GUGGCACCGAGUCGGUGCU*mU*mU*mU | |
| EF18 | Effi-sg23 | AUUUACCUUCUGGUGGCUCCGUUUUAGAGCUAGAAAUAG | 437 |
| | | CAAGUUAAAAUAAGGCUAGUCCGUUAUCAACUUGAAAAA | |
| | | GUGGCACCGAGUCGGUGCU*mU*mU*mU | |
| EF19 | LP | CUUACCUGGAUCCAUUCAUGGUUUUAGAGCUAGAAAUAG | 438 |
| | sgRNA38 | CAAGUUAAAAUAAGGCUAGUCCGUUAUCAACUUGAAAAA | |
| | | GUGGCACCGAGUCGGUGCU*mU*mU*mU | |
| EF20 | LP | CCUGCAGGAGGCUCUGUCGGGUUUUAGAGCUAGAAAUAG | 439 |
| | sgRNA39 | CAAGUUAAAAUAAGGCUAGUCCGUUAUCAACUUGAAAAA | |
| | | GUGGCACCGAGUCGGUGCU*mU*mU*mU | |
| EF21 | LP | UCACUUUAGGGAGUCUCCGGGUUUUAGAGCUAGAAAUAG | 440 |
| | sgRNA40 | CAAGUUAAAAUAAGGCUAGUCCGUUAUCAACUUGAAAAA | |
| | | GUGGCACCGAGUCGGUGCU*mU*mU*mU | |
| EF22 | LP | CCUCUGCAGACCCCACACUGGUUUUAGAGCUAGAAAUAG | 441 |
| | sgRNA41 | CAAGUUAAAAUAAGGCUAGUCCGUUAUCAACUUGAAAAA | |
| | | GUGGCACCGAGUCGGUGCU*mU*mU*mU | |
| EF23 | Effi-sg24 | CACAGGUCCUCCCCUUGGAGGUUUUAGAGCUAGAAAUAG | 442 |
| | | CAAGUUAAAAUAAGGCUAGUCCGUUAUCAACUUGAAAAA | |
| | | GUGGCACCGAGUCGGUGCU*mU*mU*mU | |
| EF24 | LP | CUUACCUCUUCAUUUCCAGGGUUUUAGAGCUAGAAAUAG | 443 |
| | sgRNA46 | CAAGUUAAAAUAAGGCUAGUCCGUUAUCAACUUGAAAAA | |
| | | GUGGCACCGAGUCGGUGCU*mU*mU*mU | |
| EF25 | LP | CUCACCUGUGAUGGGCACGUGUUUUAGAGCUAGAAAUAG | 444 |
| | sgRNA118 | CAAGUUAAAAUAAGGCUAGUCCGUUAUCAACUUGAAAAA | |
| | | GUGGCACCGAGUCGGUGCU*mU*mU*mU | |
| EF26 | LP | AACAUACUUGUCAUUGACGAGUUUUAGAGCUAGAAAUAG | 445 |
| | sgRNA119 | CAAGUUAAAAUAAGGCUAGUCCGUUAUCAACUUGAAAAA | |
| | | GUGGCACCGAGUCGGUGCU*mU*mU*mU | |
| EF27 | Effi-sg25 | UCCCAGACCAGCACAUCCUGGUUUUAGAGCUAGAAAUAG | 446 |
| | | CAAGUUAAAAUAAGGCUAGUCCGUUAUCAACUUGAAAAA | |
| | | GUGGCACCGAGUCGGUGCU*mU*mU*mU | |
| EF28 | Effi-sg26 | CCCUCCCAGCCAUGGGAACAGUUUUAGAGCUAGAAAUAG | 447 |
| | | CAAGUUAAAAUAAGGCUAGUCCGUUAUCAACUUGAAAAA | |
| | | GUGGCACCGAGUCGGUGCU*mU*mU*mU | |
| EF29 | Effi-sg16 | ACUCUAAAAAGUGAAAAUCAGUUUUAGAGCUAGAAAUAG | 448 |
| | | CAAGUUAAAAUAAGGCUAGUCCGUUAUCAACUUGAAAAA | |
| | | GUGGCACCGAGUCGGUGCU*mU*mU*mU | |

¹The symbol “*mU” incidates a uracil nucleotide with a 2′-O-methyl modification and linked to the listed 5′ nucleotide by a 3′-phosphorothioate. In some embodiments, the first three 5′-terminal nucleotides of a guide polynucleotide contain a 2′-O-methyl modification and/or the first four 5′ terminal nucleotides of a guide polynucleotide are each linked to each other by a 3′-phosphorothioate.

TABLE 2A
Exemplary spacer sequences. An exemplary adenosine base editor (ABE) is
ABE8.20m.

| | | | | Protospacer | Compatible |
|---|---|---|---|---|---|
| Guide | Alternative | | SEQ ID | Adjacent | base |
| Name | Name | Spacer | NO | Motif (PAM) | editor(s) |
| EF8 | LP sgRNA26 | AUGUACCUGGUCCAAGGAAC | 456 | | ABE/CBE |
| EF1 | LP sgRNA87 | CUCACCAGAUUCCCGAAGGU | 449 | AGG | ABE |
| EF2 | LP sgRNA88 | UCUUUAGUCUUCUCCUUGCA | 450 | TGG | ABE |
| EF3 | LP sgRNA13 | CCUCCUUACCAGCAAGAGGC | 451 | | ABE/CBE |
| EF4 | LP sgRNA14 | AUAUUACCUUUCGAUGCCGU | 452 | | ABE/CBE |
| EF5 | LP sgRNA15 | GCCCGUCAUGGAGAUGGAAA | 453 | | ABE |
| EF6 | LP sgRNA16 | GGAAACAUACCCUGUAGCAG | 454 | | ABE |
| EF7 | LP sgRNA19 | UAGGAUGGUAGCACACAACC | 455 | | ABE |
| EF9 | LP sgRNA27 | GGUUCAUACCUUGAAACAGG | 457 | | ABE |
| EF10 | LP sgRNA28 | AGGUCCUACCUUGUCAAUAA | 458 | | ABE |
| EF11 | Effi-sg17 | UUUAGGAACCUCCUUAUAGC | 459 | CGG | ABE |
| EF12 | Effi-sg18 | GCUUACUUCUUCCGGAUGAA | 460 | AGG | ABE |
| EF13 | LP sgRNA35 | CCUGCAGGACGGAGAGGAGC | 461 | | ABE |
| EF14 | Effi-sg19 | GCGGGCAUCUGGGCGCCGGG | 462 | AGG | ABE |
| EF15 | Effi-sg20 | GGACUCACCCGCUUCUGCAG | 463 | GGG | ABE |
| EF16 | Effi-sg21 | CUCCACAGGCAAAGAACAGA | 464 | AGG | ABE |
| EF17 | Effi-sg22 | CUCCAGGACGGCCGGGGCUU | 465 | TGG | ABE |
| EF18 | Effi-sg23 | AUUUACCUUCUGGUGGCUCC | 466 | AGG | ABE |
| EF19 | LP sgRNA38 | CUUACCUGGAUCCAUUCAUG | 467 | | ABE/CBE |
| EF20 | LP sgRNA39 | CCUGCAGGAGGCUCUGUCGG | 468 | | ABE |
| EF21 | LP sgRNA40 | UCACUUUAGGGAGUCUCCGG | 469 | | ABE |
| EF22 | LP sgRNA41 | CCUCUGCAGACCCCACACUG | 470 | | ABE |
| EF23 | Effi-sg24 | CACAGGUCCUCCCCUUGGAG | 471 | GGG | ABE |
| EF24 | LP sgRNA46 | CUUACCUCUUCAUUUCCAGG | 472 | | ABE/CBE |
| EF25 | LP | CUCACCUGUGAUGGGCACGU | 473 | | ABE |
| | sgRNA118 | | | | |
| EF26 | LP | AACAUACUUGUCAUUGACGA | 474 | | ABE |
| | sgRNA119 | | | | |
| EF27 | Effi-sg25 | UCCCAGACCAGCACAUCCUG | 475 | CGG | ABE |
| EF28 | Effi-sg26 | CCCUCCCAGCCAUGGGAACA | 476 | AGG | ABE |
| EF29 | Effi-sg16 | ACUCUAAAAAGUGAAAAUCA | 477 | AGG | CBE |
| EF46 | | CUUACCGAUGUUCCCUUCGA | 478 | TGG | ABE |
| EF47 | | CUUACUUCAGUGUCCUAUGC | 479 | AGG | ABE |
| EF48 | | CCCAGGCCUACCCUCUGAAG | 480 | AGG | ABE |
| EF49 | | CACCCUAGACUCCACCAAAA | 481 | AGG | ABE |
| EF50 | | AUUAUAGGCCAUUGGGUACG | 482 | AGG | ABE |
| EF51 | | UCCUAGACUCUGAAGGACGA | 483 | CGG | ABE |
| EF52 | | CCAAGGUCCAAUCACAUGUG | 484 | TGG | ABE |
| EF53 | | GGCUAGGAGUCAGCGACAUA | 485 | TGG | ABE |
| EF54 | | ACUUACCUCGCGGACAUUCC | 486 | TGG | ABE |
| EF55 | | UCCCCCAGCCAACCUUUGUA | 487 | CGG | ABE |
| EF56 | | UGCUUACCCCACCCCAGUUG | 488 | AGG | ABE |
| EF57 | | AAUCACCUCUGAACAAUCCC | 489 | TGG | ABE |
| EF58 | | GAGGCUUACCUCUUCACUGU | 490 | TGG | ABE |
| EF59 | | UUUGGACAGAUCUAUUCCAG | 491 | AGG | ABE |
| EF60 | | ACCUCACCUUCUGUUUGUCG | 492 | AGG | ABE |
| EF61 | | CCCACAGAACACAGCCCACU | 493 | TGG | ABE |
| EF62 | | UGCCCACAGGUGACAGACUU | 494 | CGG | ABE |
| EF63 | | AGCCCUCACCUCUCACUAGU | 495 | GGG | ABE |
| EF64 | | CCCAGCCGUACUAUGCCACG | 496 | AGG | ABE |
| EF65 | | AUCUCAGAGUUUGCAGAAGC | 497 | AGG | ABE |
| EF66 | | ACUGCUGACCUUGAUGUAGU | 498 | TGG | ABE |
| EF67 | | UGCGUCCAGAACCAGCUGCU | 499 | AGG | ABE |
| EF68 | | CUCACCUGUCAAGAGCGGCG | 500 | TGG | ABE |
| EF69 | | UCUUUAGUCCGAGGAUCAGA | 501 | AGG | ABE |
| EF70 | | GCAAUUACCUUGUAUGGUUG | 502 | GGG | ABE |
| EF71 | | UGUUAUAGCUACUGCUGUGU | 503 | TGG | ABE |
| EF72 | | UUUACUUACCAAUUUGGAAU | 504 | TGG | ABE |
| EF73 | | CAGCAUGAAGGCGCUGAGCC | 505 | CGG | ABE |
| EF74 | | GGCUUACCUGGAUGGGAAGG | 506 | TGG | ABE |
| EF75 | | UCAGUUUACCAUUCGGAAGC | 507 | CGG | ABE |
| EF76 | | ACUUACCAGAAUGGGUCCUG | 508 | AGG | ABE |
| EF77 | | UGCUUACCCACAAACUUUUG | 509 | TGG | ABE |
| EF85 | | GCACCAUGGCCUCGGCUCGG | 517 | CGG | ABE |
| EF86 | | CUCACUGGGUGUCUGGUCUG | 518 | CGG | ABE |
| EF87 | | CUUACCUCUUGGGACAGGAA | 519 | AGG | ABE |
| EF88 | | UGCAGGCAGAGAUUGUCAAG | 520 | AGG | ABE |
| EF89 | | UUUAUAGCACCAGCAACAAG | 521 | TGG | ABE |
| EF90 | | CCCAAUACCUUUAAUGAUCC | 522 | AGG | ABE |
| EF91 | | CCUCACCCUCAUAGCAGUCG | 523 | CGG | ABE |
| EF92 | | UCCAGGUCCAGCUGUAUGUA | 524 | TGG | ABE |
| EF93 | | CUUACUCAAAGUCCCCAUCC | 525 | CGG | ABE |
| EF94 | TSBTx1599/ | ACUCACCCAGCAUCCCCAGC | 526 | AGG | ABE |
| | JMG222 | | | | |
| EF95 | | GCUUACAGAUCUUGCCCCGC | 527 | AGG | ABE |
| EF96 | | AUUUACCUGAACCUCUGAAU | 528 | TGG | ABE |
| EF97 | | CUUAGCAUCAAGGCAUGCCA | 529 | TGG | ABE |
| EF98 | | CUUACUGUACAACAAGCUGC | 530 | TGG | ABE |
| EF99 | | CCCUUACCAGGCUUGAUGAG | 531 | AGG | ABE |
| EF100 | | CUCAGACAAGGACACGUUGA | 532 | AGG | ABE |
| EF101 | | CAACAGGAAGGCUGACGAGG | 533 | TGG | ABE |
| EF102 | | CCCAGUGAAGACAGCAUCAU | 534 | CGG | ABE |
| EF103 | | CCCAGGACUUCCUGCUGCUG | 535 | CGG | ABE |
| EF104 | | UGUGUUACCUUGAUGACCGG | 536 | CGG | ABE |
| EF105 | | GUUCAUAGCUGGGCUCCUGG | 537 | AGG | ABE |
| EF106 | | CCUACCCACCUCCUUUCUCA | 538 | GGG | ABE |
| EF107 | | ACUCACGUGAGCACCGGGAU | 539 | GGG | ABE |
| EF108 | | GACUCACGUGAGCACCGGGA | 540 | TGG | ABE |
| EF109 | | GGACUCACUGAGACAAAGUA | 541 | GGG | ABE |
| EF110 | | GACUCACUGAGACAAAGUAG | 542 | GGG | ABE |
| EF111 | | CUCUUCCAGCCAGCUGAGGU | 543 | GGG | ABE |
| EF112 | | GAACUCACUCUUGUCAGUCU | 544 | TGG | ABE |
| EF113 | | UCCAGCGCUAGUCCUGCUGA | 545 | TGG | ABE |
| EF114 | | GAGGCAUGGCUGAAAUCUUC | 546 | CGG | ABE |
| EF115 | | GACUCUACCUGUUUGCCAGG | 547 | GGG | ABE |
| EF116 | | ACUUACCAGGUGCAGGGUGU | 548 | CGG | ABE |
| EF117 | | GCUGUGUACCUGAGGGGUCC | 549 | GGG | ABE |
| EF118 | | UUUGUAGCCAUCCAGUCCAA | 550 | TGG | ABE |
| EF119 | | UUCCUACAGAUUCCUCCUUA | 551 | TGG | ABE |
| EF120 | | AUACUCACAGGCAAGCUGGA | 552 | GGG | ABE |

TABLE 2B
Description of sequences targeted by spacers of Table 2A.

| | | | | Molecular |
|---|---|---|---|---|
| | | | | Weight of |
| | | | | Polypeptide |
| | | | | Encoded by |
| | | | Target | Target |
| Guide | | Description of | Alteration | Polypeptide |
| Name | Alternative Name | Target | Location¹ | (kDa) |
| EF1 | LP sgRNA87 | CISH | CISH Ex. 3 SA | 29 |
| | | | A4/A5 | |
| EF2 | LP sgRNA88 | CBLB | CBLB Ex. 9 SD | 109 |
| | | | A6 | |
| EF3 | LP sgRNA13 | PTPN1 (PTP1B) | | 50 |
| EF4 | LP sgRNA14 | PTPN1 (PTP1B) | A6 | |
| EF5 | LP sgRNA15 | PTPN1 (PTP1B) | sgRNA3 (ABE) | |
| EF6 | LP sgRNA16 | PTPN1 (PTP1B) | sgRNA8 (ABE) | |
| EF7 | LP sgRNA19 | SOCS1 | Start Co. A5 | 23 |
| EF8 | LP sgRNA26 | R3CH1 (Roquin- | Ex. 3 SD A5 | 130 |
| | | 1) | | |
| EF9 | LP sgRNA27 | R3CH1 (Roquin- | Ex. 4 SD A8 | |
| | | 1) | | |
| EF10 | LP sgRNA28 | R3CH1 (Roquin- | Ex. 5 SD A8 | |
| | | 1) | | |
| EF11 | Effi-sg17 | R3CH1 (Roquin- | Ex. 12 SD A4 | |
| | | 1) | | |
| EF12 | Effi-sg18 | R3CH1 (Roquin- | Ex. 12 SA A5 | |
| | | 1) | | |
| EF13 | LP sgRNA35 | DNMT3A | Ex. 2 SD A6 | 102 |
| EF14 | Effi-sg19 | DNMT3A | Start Co. A7 | |
| EF15 | Effi-sg20 | DNMT3A | Ex. 3 SA A7 | |
| EF16 | Effi-sg21 | DNMT3A | Ex. 4 SD A7 | |
| EF17 | Effi-sg22 | DNMT3A | Ex. 7 SD A5 | |
| EF18 | Effi-sg23 | DNMT3A | Ex. 9 SA A6 | |
| EF19 | LP sgRNA38 | FLI-1 | Ex. 2 SD A4 | 51 |
| EF20 | LP sgRNA39 | FLI-1 | Ex. 2 SA A6 | |
| EF21 | LP sgRNA40 | FLI-1 | Ex. 3 SD A8 | |
| EF22 | LP sgRNA41 | FLI-1 | Ex. 4 SD A8 | |
| EF23 | Effi-sg24 | FLI-1 | Ex. 6 SD A4 | |
| EF24 | LP sgRNA46 | DDIT3 (Chop) | Ex. 1 SA A4 | 20 |
| EF25 | LP sgRNA118 | ZC3H12A | Ex. 2 SA A4, C5 | 66 |
| | | (Regnase1) | | |
| EF26 | LP sgRNA119 | ZC3H12A | Ex. 3 SA A6, C7 | |
| | | (Regnase1) | | |
| EF27 | Effi-sg25 | ZC3H12A | Ex. 3 SD A5 | |
| | | (Regnase1) | | |
| EF28 | Effi-sg26 | ZC3H12A | Ex. 2 SD A8 | |
| | | (Regnase1) | | |
| EF29 | Effi-sg16 | PTPN2 | Ex. 6 SD C4 | |
| EF46 | | DCK | Ex. 1 SA A4 | 31 |
| EF47 | | DCK | Ex. 5 SA A4 | |
| EF48 | | DGKa | Ex. 2 SD A4 | 83 |
| EF49 | | DGKa | Ex. 3 SD A7 | |
| EF50 | | DGKa | Ex. 4 SD A6 | |
| EF51 | | DGKa | Ex. 7 SD A5 | |
| EF52 | | DGKa | Ex. 9 SD A4 | |
| EF53 | | DGKz | Ex. 3 SD A5 | 124 |
| EF54 | | DGKz | Ex. 5 SA A5 | |
| EF55 | | DGKz | Ex. 6 SD A7 | |
| EF56 | | DGKz | Ex. 13 SA A6 | |
| EF57 | | PRDM1 | Ex. 2 SA A5 | 95-100 |
| EF58 | | PRDM1 | Ex. 3 SA A8 | |
| EF59 | | PRDM1 | Ex. 4 SD A8 | |
| EF60 | | PRKACA | Ex. 3 SA A6 | 42 |
| EF61 | | PRKACA | Ex. 3 SD A6 | |
| EF62 | | PRKACA | Ex. 7 SD A8 | |
| EF63 | | PTPN6 | Ex. 3 SA A8 | 68 |
| EF64 | | PTPN6 | Ex. 6 SD A4 | |
| EF65 | | PTPN6 | Ex. 7 SD A6 | |
| EF66 | | PTPN6 | Ex. 8 SA A8 | |
| EF67 | | PTPN6 | Ex. 8 SD A8 | |
| EF68 | | EIF2A | Ex. 1 SA A4 | ~70 |
| EF69 | | EIF2A | Ex. 2 SD A6 | |
| EF70 | | EIF2A | Ex. 7 SA A7 | |
| EF71 | | EIF2A | Ex. 9 SD A7 | |
| EF72 | | EIF2A | Ex. 10 SA A8 | |
| EF73 | | ID3 | Start Co. A5 | 13 |
| EF74 | | ID3 | Ex. 1 SA A6 | |
| EF75 | | IKZF2 | Ex. 3 SA A8 | 58, 63-70, 85 |
| EF76 | | IKZF2 | Ex. 5 SA A5 | |
| EF77 | | IKZF2 | Ex. 7 SA A6 | |
| EF85 | | SOX4 | Start Co. A6 | |
| EF86 | | TLE4 | Ex. 1 SA A4 | 84 |
| EF87 | | TLE4 | Ex. 5 SA A4 | |
| EF88 | | TLE4 | Ex. 5 SD A4 | |
| EF89 | | TLE4 | Ex. 6 SD A6 | |
| EF90 | | TLE4 | Ex. 7 SA A7 | |
| EF91 | | TMEM184B | Ex. 3 SA A5 | 12 |
| EF92 | | TMEM184B | Ex. 5 SD A4 | |
| EF93 | | TMEM184B | Ex. 6 SA A4 | |
| EF94 | TSBTx1599/JMG222 | CD5 | Ex. 1 SA A5 | |
| EF95 | | RASA2 | Ex. 1 SA A5 | |
| EF96 | | RASA2 | Ex. 4 SA A5 | |
| EF97 | | RASA2 | Ex. 7 SD A4 | |
| EF98 | | RASA2 | Ex. 6 SA A4 | |
| EF99 | | RASA2 | Ex. 9 SA A6 | |
| EF100 | | DHX37 | | 130 |
| EF101 | | DHX37 | | |
| EF102 | | DHX37 | | |
| EF103 | | DHX37 | | |
| EF104 | | DHX37 | | |
| EF105 | | IL6 | Start Co. A5 | |
| EF106 | | IL6 | Ex. 1 SA A8 | |
| EF107 | | TMEM222 | | |
| EF108 | | TMEM222 | | |
| EF109 | | TMEM222 | | |
| EF110 | | TMEM222 | | |
| EF111 | | PFN1 | Ex. 2 SA A8 | 15 |
| EF112 | | PFN1 | Ex. 2 SD A7 | |
| EF113 | | PFN1 | Ex. 3 SA A4 | |
| EF114 | | BATF | Start co. A6 | 15 |
| EF115 | | BATF | Ex. 1 SD A7 | |
| EF116 | | BATF | Ex. 2 SD A5 | |
| EF117 | | ARID1A | Ex. 1 SD A8 | 270 |
| EF118 | | ARID1A | Ex. 2 SA A6 | |
| EF119 | | ARID1A | Ex. 3 SA A8 | |
| EF120 | | ARID1A | Ex. 4 SD A7 | |

¹The term “SA” indicates a splice acceptor site, the term “SD” indicates a splice donor site, the term “Ex.” Indicates an exon, the term “Start Co.” indicates a start codon, and the term “NX,” where N is any nucleotide and X is an integer, indicates the location within the spacer (relative to the 5′-end of the spacer) corresponding to the target nucleobase.

TABLE 2C
Further exemplary spacer sequences.

| Polypeptide | | | |
|---|---|---|---|
| endoded by | | | Compatible |
| target | | SEQ | base |
| polynucleotide | Spacer | ID NO | editor(s) |
| B2M | AAGUCAACUUCAAUGUCGGA | 681 | CBE, ABE |
| B2M | ACAAAGUCACAUGGUUCACA | 682 | CBE, ABE |
| B2M | ACAGCCCAAGAUAGUUAAGU | 683 | CBE |
| B2M | ACCCAGACACAUAGCAAUUC | 684 | CBE, ABE |
| B2M | ACUCACGCUGGAUAGCCUCC | 685 | CBE, ABE |
| B2M | ACUUGUCUUUCAGCAAGGAC | 686 | CBE, ABE |
| B2M | AGUCACAUGGUUCACACGGC | 687 | CBE, ABE |
| B2M | AUACUCAUCUUUUUCAGUGG | 688 | CBE, ABE |
| B2M | CACAGCCCAAGAUAGUUAAG | 689 | CBE |
| B2M | CAGCCCAAGAUAGUUAAGUG | 690 | CBE |
| B2M | CAGUAAGUCAACUUCAAUGU | 691 | CBE, ABE |
| B2M | CAUACUCAUCUUUUUCAGUG | 692 | CBE, ABE |
| B2M | CGCGAGCACAGCUAAGGCCA | 693 | CBE, ABE |
| B2M | CGUGAGUAAACCUGAAUCUU | 694 | CBE |
| B2M | CUCACGCUGGAUAGCCUCC | 695 | CBE, ABE |
| B2M | CUCAGGUACUCCAAAGAUUC | 696 | |
| B2M | CUUACCCCACUUAACUAUCU | 697 | CBE, ABE |
| B2M | GAAGUUGACUUACUGAAGAA | 698 | CBE, ABE |
| B2M | GAGUAGCGCGAGCACAGCUA | 699 | CBE, ABE |
| B2M | GCAUACUCAUCUUUUUCAGU | 700 | CBE, ABE |
| B2M | GCUACUCUCUCUUUCUGGCC | 701 | CBE, ABE |
| B2M | GGCAUACUCAUCUUUUUCAG | 702 | CBE, ABE |
| B2M | UACCCCACUUAACUAUCU | 703 | |
| B2M | UCACGCUGGAUAGCCUCC | 704 | CBE, ABE |
| B2M | UCGAUCUAUGAAAAAGACAG | 705 | ABE |
| B2M | UGGAGAGAGAAUUGAAAAAG | 706 | CBE, ABE |
| B2M | UGGAGUACCUGAGGAAUAUC | 707 | |
| B2M | UGGGCUGUGACAAAGUCACA | 708 | CBE, ABE |
| B2M | UUACCCCACUUAACUAUCUU | 709 | CBE, ABE |
| B2M | UUCAGACUUGUCUUUCAGCA | 710 | CBE, ABE |
| B2M | UUUGACUUUCCAUUCUCUGC | 711 | CBE, ABE |
| CD3e | ACACACUGUGGGGGGUGGGG | 712 | |
| CD3e | ACACAGACACGUGAGUUUAU | 713 | |
| CD3e | ACACUGUGGGGGGUGGGGUG | 714 | |
| CD3e | ACUCACCUGAUAAGAGGCAG | 715 | |
| CD3e | CACACUGUGGGGGGUGGGGU | 716 | |
| CD3e | CCGACUGCAUCUUUGUUUCA | 717 | |
| CD3e | CGUUACCUCAUAGUCUGGGU | 718 | |
| CD3e | CUGGAUUACCUCUUGCCCUC | 719 | |
| (TSBTx4073) | | | |
| CD3e | GUUACCUCAUAGUCUGGGUU | 720 | |
| CD3e | UACCACCUGAAAAUGAAAAA | 721 | |
| CD3e | UAUAUGCUGGGGAGAAAGAA | 722 | |
| CD3e | UUAUAUGCUGGGGAGAAAGA | 723 | |
| CD3e | UUGUCCUGCGGAGGAAGGAG | 724 | |
| CD3e | UUUGUCCUGCGGAGGAAGGA | 725 | |
| CD3e | UUUUGUCCUGCGGAGGAAGG | 726 | |
| CD3g | ACAUACUUCUGUAAUACACU | 727 | |
| CD3g | ACCUUCAAGGAAACCAGUUG | 728 | |
| CD3g | CAUGGAACAGGGGAAGGGCC | 729 | |
| CD3g | CUCUUCCAUUGGGUACAUAA | 730 | |
| CD3g | CUUCAAGGUAAGGGCCUACU | 731 | |
| CD3g | GACCAGUACAGCCACCUUCA | 732 | |
| CD3g | UCUCCUACCUUUGAUUGACU | 733 | |
| CD3g | UGACCAGCUCUACCAGGUAA | 734 | |
| CD3g | UGACUAUCAAGAAGAUGGUU | 735 | |
| CD3g | UGGCCCAGUCAAUCAAAGGU | 736 | |
| CD3g | UUCUCCUACCUUUGAUUGAC | 737 | |
| CD3g | UUUAAACCAUGUGAUAUUUU | 738 | |
| CD7 | CccUACCUGUCACCAGGACC | 739 | |
| CIITA | AAAGGCACUGCAAGAGACAA | 740 | |
| CIITA | ACACUCACUCCAUCACCCGG | 741 | |
| CIITA | ACAUCAAAGUACCCUACAGG | 742 | |
| CIITA | ACCGGCUCUGCAAAGGCCAG | 743 | |
| CIITA | ACCUCCCGAGCAAACAUGAC | 744 | |
| CIITA | ACUGGACCAGUAUGUCUUCC | 745 | |
| CIITA | AGACUCAGAGGUGAGAGGAG | 746 | |
| CIITA | AGCCAAGUACCCCCUCCCAG | 747 | |
| CIITA | AGCCCCAAGGUAAAAAGGCC | 748 | |
| CIITA | AGCCUAGGAGGCAAAGAGCA | 749 | |
| CIITA | AGGCCAUUUUGGAAGCUUGU | 750 | |
| CIITA | AGGCUGCAGGUGGAAUCAGA | 751 | |
| CIITA | CACAUCCUGCAAGGGGGGAU | 752 | |
| CIITA | CACUCACCUUAGCCUGAGCA | 753 | |
| CIITA | CACUCACUCCAUCACCCGGA | 754 | |
| CIITA | CACUCACUUGAGGGUUUCCA | 755 | |
| CIITA | CAGACAUCAAAGUACCCUAC | 756 | |
| CIITA | CAGACUGCGGGGACACAGUG | 757 | |
| CIITA | CAGCUCACAGUGUGCCACCA | 758 | |
| CIITA | CCACAUCCUGCAAGGGGGGA | 759 | |
| CIITA | CCACUCACCUUAGCCUGAGC | 760 | |
| CIITA | CCCACCCAAUGCCCGGCAGC | 761 | |
| CIITA | CCCCCAGGCUUUCCCCAAAC | 762 | |
| CIITA | CCUCCUGCAAUGCUUCCUGG | 763 | |
| CIITA | CCUGGUCCAGAGCCUGAGCA | 764 | |
| CIITA | CCUUACCUGUCAUGUUUGCU | 765 | |
| CIITA | CGCCCAGGUCCUCACGUCUG | 766 | |
| CIITA | CGUCCAGUACAACAAGUUCA | 767 | |
| CIITA | CUCUGGCAAAUCUCUGAGGC | 768 | |
| CIITA | CUGCCAAAUUCCAGCCUCCU | 769 | |
| CIITA | CUGGUCAGGGCAAGAGCUAU | 770 | |
| CIITA | CUUAGUCCAACACCCACCGC | 771 | |
| CIITA | CUUCCCCCAGCUGAAGUCCU | 772 | |
| CIITA | GAACGGCAGCUGGCCCAAGG | 773 | |
| CIITA | GACACGAGUGAUUGCUGUGC | 774 | |
| CIITA | GACCAGAUUCCCAGUAUGUU | 775 | |
| CIITA | GAGCCAGCCACAGGGCCCCC | 776 | |
| CIITA | GAGCCCCAAGGUAAAAAGGC | 777 | |
| CIITA | GCGUCCACAUCCUGCAAGGG | 778 | |
| CIITA | GGAAGCAGAAGGUGCUUGCG | 779 | |
| CIITA | GGCCCAAGGAGGCCUGGCUG | 780 | |
| CIITA | GGCGGGCCAAGACUUCUCCC | 781 | |
| CIITA | GGCGUCCACAUCCUGCAAGG | 782 | |
| CIITA | GGCUGCAGCCGGGGACACUG | 783 | |
| CIITA | GGGCCCACAGCCACUCGUGG | 784 | |
| CIITA | GGGCGUCCACAUCCUGCAAG | 785 | |
| CIITA | GUACAAGCUGUCGGAAACAG | 786 | |
| CIITA | UAACAUACUGGGAAUCUGGU | 787 | |
| CIITA | UAUGACCAGAUGGACCUGGC | 788 | |
| CIITA | UCACUCCAGAUGCUGCAGGG | 789 | |
| CIITA | UGCUCUGGAGAUGGAGAAGC | 790 | |
| CIITA | UGGGCGUCCACAUCCUGCAA | 791 | |
| CIITA | UGGUGCAGGCCAGGCUGGAG | 792 | |
| CIITA | UGUCUUCCAGGACUCCCAGC | 793 | |
| CIITA | UUCAACCAGGAGCCAGCCUC | 794 | |
| CIITA | UUCCAGAAGAAGCUGCUCCG | 795 | |
| CIITA | UUUUACCUUGGGGCUCUGAC | 796 | |
| PD1 | ACGACUGGCCAGGGCGCCUG | 797 | |
| PD1 | CACCUACCUAAGAACCAUCC | 798 | |
| PD1 | GGACCCAGACUAGCAGCACC | 799 | |
| PD1 | GGAGUCUGAGAGAUGGAGAG | 800 | |
| PD1 | GGGGUUCCAGGGCCUGUCUG | 801 | |
| PD1 | CCUCCUUCUUUGAGGAGAAA | 802 | |
| PD1 | CCAGUGGCGAGAGAAGACCC | 803 | |
| PD1 | UGCCCAGCCACUGAGGCCUG | 804 | |
| PD1 | CGACUGGCCAGGGCGCCUGU | 805 | |
| PD1 | ACCGCCCAGACGACUGGCCA | 806 | |
| PD1 | CACCGCCCAGACGACUGGCC | 807 | |
| PD1 | UUCUCUCUGGAAGGGCACAA | 808 | |
| PD1 | CUACAACUGGGCUGGCGGCC | 809 | |
| PD1 | GACGUUACCUCGUGCGGCCC | 810 | |
| PD1 | CAGCAACCAGACGGACAAGC | 811 | |
| PD1 | CCUGCAGAGAAACACACUUG | 812 | |
| PD1 | CAGUUCCAAACCCUGGUGGU | 813 | |
| PD1 | GUGUCACACAACUGCCCAAC | 814 | |
| PD1 | AGCCGGCCAGUUCCAAACCC | 815 | |
| PD1 | CGGCCAGUUCCAAACCCUGG | 816 | |
| PD1 | UCCCUGCAGAGAAACACACU | 817 | |
| PD1 | GAGACUCACCAGGGGCUGGC | 818 | |
| TRAC | CCAGCCAAGUACGUAAGUAG | 819 | |
| TRAC | CUGGAUAUCUGUGGGACAAG | 820 | |
| TRAC | CUUACCUGGGCUGGGGAAGA | 821 | |
| TRAC | GCUACAAACAAGCUCAUCUU | 822 | |
| TRAC | UUCAAAACCUGUCAGUGAUU | 823 | |
| TRAC | UUCGUAUCUGUAAAACCAAG | 824 | |
| TRAC | UUUCAAAACCUGUCAGUGAU | 825 | |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain may comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2cl (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that may comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 may comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein may comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein may comprise a deaminase flanked by an N-terminal fragment and a C-terminal fragment of a Cas9 or Cas12 (e.g., Cas12b/C2c1), polypeptide

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes may comprise more than one deaminase. The fusion protein or complex may comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein may comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2cl, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:
ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion | Inserted |
|---|---|---|
| | site | between aa |
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA.

For example, the base editor may comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g. ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants. Alterations are referenced to TaadA*7.10.
Additional details of TadA*9 adenosine deaminases are described in
International PCT Application No. PCT/US2020/049975, which is incorporated
herein by reference in its entirety for all purposes.

| TadA*9 Description | Alterations |
|---|---|
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K_V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K_V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R1471D, F149Y, T1661, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of MI, MIS, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, I49G, I49H, I49V, I49F, I49H, I49I, I49M, I49N, I49K, I49Q, I49T, G50L, G50S, G50R, G50G, R51H, R51L, R51N, L51W, R51Y, R51G, R51V, R51R, H52D, H52Y, H52I, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E591, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q711, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, I76I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, I95S, I95G, I95L, I95H, I95V, H96A, H96L, H96R, H96S, H96H, H96N, H96E, S97C, S97G, S97I, S97M, S97R, S97S, S97P, R98K, R98I, R98N, R98Q, R98G, R98H, R98C, R98L, R98R, G100R, G100V, G100K, G100A, G100S, G100M, G100I, R101V, R101R, R101S, R101C, V102A, V102F, V1021, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K1101, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M1260, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E1311, E131V, E131K, I132I, I132F, I132T, I132L, I132V, I132E, T133V, T133E, T133G, T133K, T133T, T133A, T133H, T133F, T133I, E134A, E134E, E134G, E1341, E134H, E134K, E134T, G135G, G135V, G135I, G135P, G135E, I136G, I136L, I136T, I136I, I137A, I137D, I137E, L137M, I137S, L137L, L137I, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D1471, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q1541, Q154A, Q154K, E155F, E155G, E1551, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K1611, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D1671, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminse base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor may comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor may comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor may comprise one or more mutations selected from the group consisting of H121R, H122R, R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (TADC)” domains. In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, I49K, I49M, I49N, I49Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+149N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to TadA*8.20.
“S” indicates “Surface,” and “NAS” indicates “Near Active Site.”
Adenosine Deaminase Variants. Mutations are indicated with reference to TadA*8.20.
“S” indicates “Surface,” and “NAS” indicates “Near Active Site.”

| | location in structure | | | | | | | | | | |
|---|---|---|---|---|---|---|---|---|---|---|---|
| | N/A | S h1 | S h1 | S h1 | NAS | NAS | NAS | NAS | | S | |
| | Amino Acid No. | | | | | | | | | | |
| | (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | I | |
| TadA*8.20 | S | H | R | T | E | R | A | I | G | Y | D |
| TadA*8.19 | | | | | | | | | | I | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

Adenosine Deaminase Variants. Mutations are indicated with reference to TadA*8.20.
“I” indicates “Internal,” “S” indicates “Surface,” and “NAS” indicates “Near Active Site.”

| | location in structure | | | | | | | | | | | |
|---|---|---|---|---|---|---|---|---|---|---|---|---|
| | I | NAS | | NAS | S | | S | S | S | | S | |
| | Amino Acid No. | | | | | | | | | | | |
| | (*START Met is AA# 1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

TABLE 6B
Adenosine deaminase variants. Mutations are
indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | L | K | T | | C | | | |
| S1.25 | S | | L | K | T | | | H | | |
| S1.26 | S | | L | K | T | | | | M | |
| S1.27 | S | | L | K | T | | | | | E |
| S1.28 | S | | L | K | T | | C | H | | |
| S1.29 | S | | L | K | T | | C | | M | |
| S1.30 | S | | L | K | T | | C | | | E |
| S1.31 | S | | L | K | T | | | H | | E |
| S1.32 | S | | L | K | T | | | | M | E |
| S1.33 | S | | L | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | I | K | T | L | C | | | |
| S1.58 | S | | I | K | T | L | | H | | |
| S1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | I | K | T | L | | H | | E |
| S1.65 | S | | I | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | C | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| S1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| S1.98 | | K | | K | T | | | | M | E |
| S1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | I | K | T | | C | | | E |
| S1.108 | | K | I | K | T | | | H | | E |
| S1.109 | | K | I | K | T | | | | M | E |
| S1.110 | | K | I | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | I | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | I | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | E |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | E |
| S1.153 | H | | | K | T | | | | M | E |
| S1.154 | H | | | K | T | | C | H | M | E |
| S1.155 | S | | | | T | | | | | |
| S1.156 | S | | | | T | | C | | | |
| S1.157 | S | | | | T | | | H | | |
| S1.158 | S | | | | T | | | | M | |
| S1.159 | S | | | | T | | | | | E |
| S1.160 | S | | | | T | | C | H | | |
| S1.161 | S | | | | T | | C | | M | |
| S1.162 | S | | | | T | | C | | | E |
| S1.163 | S | | | | T | | | H | | E |
| S1.164 | S | | | | T | | | | M | E |
| S1.165 | S | | | | T | | C | H | M | E |
| S1.166 | | A | | | T | | | | | |
| S1.167 | | A | | | T | | C | | | |
| S1.168 | | A | | | T | | | H | | |
| S1.169 | | A | | | T | | | | M | |
| S1.170 | | A | | | T | | | | | E |
| S1.171 | | A | | | T | | C | H | | |
| S1.172 | | A | | | T | | C | | M | |
| S1.173 | | A | | | T | | C | | | E |
| S1.174 | | A | | | T | | | H | | E |
| S1.175 | | A | | | T | | | | M | E |
| S1.176 | | A | | | T | | C | H | M | E |
| S1.177 | S | | I | | T | | | | | |
| S1.178 | S | | I | | T | | C | | | |
| S1.179 | S | | I | | T | | | H | | |
| S1.180 | S | | I | | T | | | | M | |
| S1.181 | S | | I | | T | | | | | E |
| S1.182 | S | | I | | T | | C | H | | |
| S1.183 | S | | I | | T | | C | | M | |
| S1.184 | S | | I | | T | | C | | | E |
| S1.185 | S | | I | | T | | | H | | E |
| S1.186 | S | | I | | T | | | | M | E |
| S1.187 | S | | I | | T | | C | H | M | E |
| S1.188 | | A | I | | T | L | | | | |
| S1.189 | | A | I | | T | L | C | | | |
| S1.190 | | A | I | | T | L | | H | | |
| S1.191 | | A | I | | T | L | | | M | |
| S1.192 | | A | I | | T | L | | | | E |
| S1.193 | | A | I | | T | L | C | H | | |
| S1.194 | | A | I | | T | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminse variants. Mutations are indicated
with reference to variant 1.2 (Table 6A) .

| | Alternative | Residue identity | | | | | | | | |
|---|---|---|---|---|---|---|---|---|---|---|
| | Variant | (START Met is amino acid #1) | | | | | | | | |
| Variant Name | Names | 4 | 6 | 17 | 23 | 76 | 77 | 100 | 111 | 114 |
| Reference | 1.2 | V | F | T | R | I | D | G | T | A |
| | (see Table 6A) | | | | | | | | | |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | W | | H | | | | |
| TadAC2.23 | pDKL-157; 2.23 | S | | | | Y | | | | |
| TadAC2.24 | pDKL-158; 2.24 | | | | | | | | | |

| | Alternative | Residue identity | | | | | | | | |
|---|---|---|---|---|---|---|---|---|---|---|
| | Variant | (START Met is amino acid #1) | | | | | | | | |
| Variant Name | Names | 119 | 122 | 127 | 143 | 147 | 158 | 159 | 162 | 166 |
| Reference | 1.2 | D | H | N | A | R | A | Q | A | T |
| | (see Table 6A) | | | | | | | | | |
| TadAC2.1 | pDKL-135; 2.1 | | | | | | | | | |
| TadAC2.2 | pDKL-136; 2.2 | | | | | | | | | |
| Reference | 1.2 | D | H | N | A | R | A | Q | A | T |
| | (see Table 6A) | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| TadA*8.20 | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| TadA*8.20 | F | E | I | Y | D | S | R | G | A | G | D | H | N | A | A |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | G | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| TadA*8.20 | F | E | I | Y | D | S | R | G | A | G | D | H | N | A | A |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | M | G | T | C | H | C | M | | G | | E | |
| S2.47 | Y | H | K | W | G | T | C | H | C | M | | | P | E | |
| S2.48 | Y | H | K | M | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | M | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| TadA*7.10 | I | V | A | T | D | H | Y | Y | F | Q | T | D |
| TadA*8e | | | S | R | N | N | | D | Y | | I | N |
| TadA*8.20 | Y | S | | | | | H | R | | R | | |
| TadA*8.17 | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| | AA positions: | | | | | | | | | | | | |
|---|---|---|---|---|---|---|---|---|---|---|---|---|---|
| | 17 | 27 | 48 | 49 | 76 | 82 | 84 | 118 | 142 | 147 | 149 | 166 | 167 |
| ABE8.19 m/ | T | E | A | I | Y/I | S | F | M | A | Y | F | T | D |
| 8.20 m | | | | | | | | | | | | | |
| 1.1 + | | H | | | I | | M | | | | Y | | |
| 8e(B879) | | | | | | | | | | | | | |
| 1.2 + | | H | | K | I | | | | | | Y | | |
| 8e(B879) | | | | | | | | | | | | | |
| 1.12 + | A | H | | M | I | | | L | | | Y | | |
| 8e(B879) | | | | | | | | | | | | | |
| 1.17 + | A | | G | | | T | | | E | | Y | | |
| 8e(B879) | | | | | | | | | | | | | |
| 1.18 + | | G | | | | | | | | | Y | | |
| 8e(B879) | | | | | | | | | | | | | |
| 1.19 + | | G | | N | | | | | | | Y | | |
| 8e(B879) | | | | | | | | | | | | | |
| 1.1 + | | H | | | I | | M | | | D | Y | I | N |
| 8e(B882) | | | | | | | | | | | | | |
| 1.2 + | | H | | K | I | | | | | D | Y | I | N |
| 8e(B882) | | | | | | | | | | | | | |
| 1.12 + | A | H | | M | I | | | L | | D | Y | I | N |
| 8e(B882) | | | | | | | | | | | | | |
| 1.17 + | A | | G | | | T | | | E | D | Y | I | N |
| 8e(B882) | | | | | | | | | | | | | |
| 1.18 + | | G | | | | | | | | D | Y | I | N |
| 8e(B882) | | | | | | | | | | | | | |
| 1.19 + | | G | | N | | | | | | D | Y | I | N |
| 8e(B882) | | | | | | | | | | | | | |

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide may comprise a CRISPR RNA (crRNA) and a trans-activating CRISPR RNA (tracrRNA) or may comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides may comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are separated in some embodiments by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide may comprise one or more modifications to provide a nucleic acid with a new or enhanced feature. A guide polynucleotide may comprise a nucleic acid affinity tag. A guide polynucleotide may comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NL)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise the amino acid sequence EGADKRTADGSEFESPKKKRKV (amino acids 8 to 29 of SEQ ID NO: 328). In some embodiments, the NLS is at a C-terminal portion of the adenosine base editor. In some embodiemtns, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U:G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure may comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences. In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10 + L36H + V82G + Y147D + F149Y + |
| | | Q154S + N157K + D167N |
| ABE-827m | MSP827 | monomer_TadA*7.10 + L36H + I76Y + V82G + Y147T + |
| | | Q154S + N157K |
| ABE-828m | MSP828 | monomer_TadA*7.10 + I76Y + V82G + Y147D + F149Y + |
| | | Q154S + D167N |
| ABE-829m | MSP829 | monomer_TadA*7.10 + L36H + I76Y + V82G + Y147D + |
| | | F149Y + Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + |
| | | Y147T + Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + |
| | | Y147D + F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)n (SEQ ID NO: 246), (GGGGS)n (SEQ ID NO: 247), and (G)n to more rigid linkers of the form (EAAAK)n (SEQ ID NO: 248), (SGGS)n (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.
Nucleic Acid Programmable DN4 Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure may comprise any domain, feature or amino acid sequence which facilitates the editing of a target polynucleotide sequence.

### Methods of Using Fusion Proteins or Complexes Comprising a Cytidine or Adenosine Dearminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein may comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Polypeptides in a Host Cell

Polypeptides of the present disclosure may be expressed in virtually any host cell of interest, including mammalian cells (e.g., human cells). In some embodiments, the host cell is an immune cell (e.g., T- or NK-cell). In some embodiments, the host cell is an allogeneic immune cell (e.g., T- or NK-cell). In some embodiments, the host cell is a CAR-T cell.

An expression vector containing a DNA encoding a nucleic acid sequence-recognizing module and/or a nucleic acid base converting enzyme can be produced, for example, by linking the DNA to the downstream of a promoter in a suitable expression vector.

In some embodiments, the nucleic acid sequence is inserted into the genome of the cell (e.g., T cell or NK cell) by introducing a vector, for example, a viral or non-viral vector, comprising the nucleic acid. Examples of viral vectors include, but are not limited to, adeno-associated viral (AAV) vectors, retroviral vectors or lentiviral vectors. In some embodiments, the lentiviral vector is an integrase-deficient lentiviral vector. In some embodiments, the nucleic acid sequence is inserted into the genome of the cell (e.g., T cell) via non-viral delivery. In non-viral delivery methods, the nucleic acid can be naked DNA, or in a non-viral plasmid or vector.

Regarding the promoter to be used, any promoter appropriate for a host to be used for gene expression can be used. For example, when the host is an animal cell, an SRα promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK), MND (a synthetic promoter that contains the U3 region of a modified MoMuLV LTR with myeloproliferative sarcoma virus enhancer) promoter, and the like can be used. Of these, CMV promoter, SR.alpha. promoter and the like may advantageously be employed in some embodiments.

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors. Non-limiting examples of viral vectors include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas9 nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (lssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632 or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N-[N-terminal portion of the split Cas9]-[intein-N]-C. In some embodiments, an intein-C is fused to the N-terminus of the C-terminal portion of the split Cas9, i.e., to form a structure of N-[intein-C]-[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of an autoimmune disease or a neoplasia (e.g., a lymphoma) in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of the embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Base Edited Chimeric Antigen Receptor (CAR) T Cells Showed Improved Function Relative to Unedited Cells after being Exposed to the Target Antigen

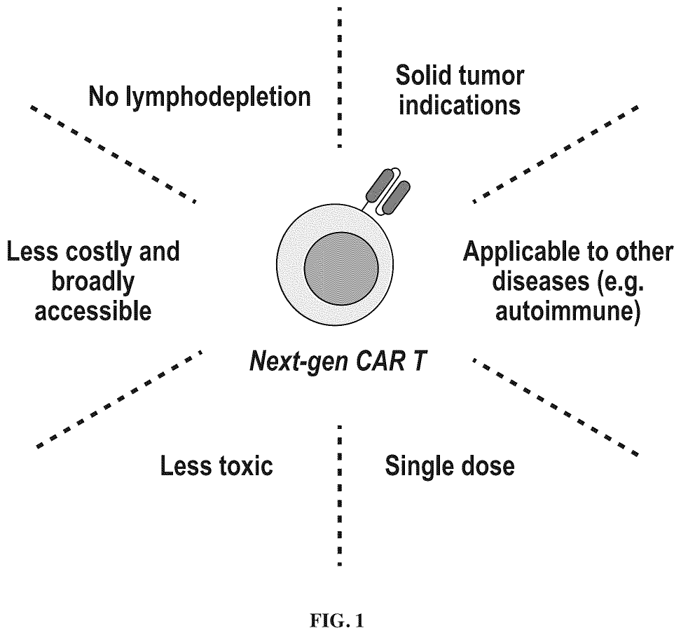

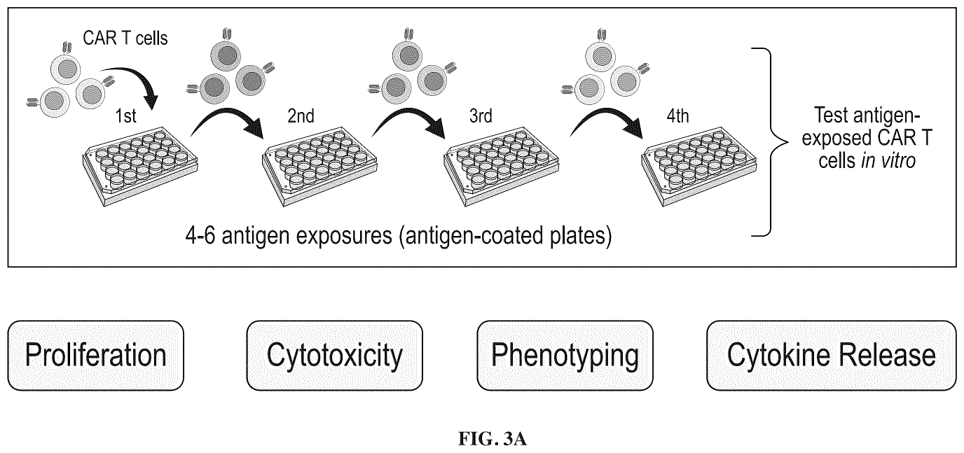

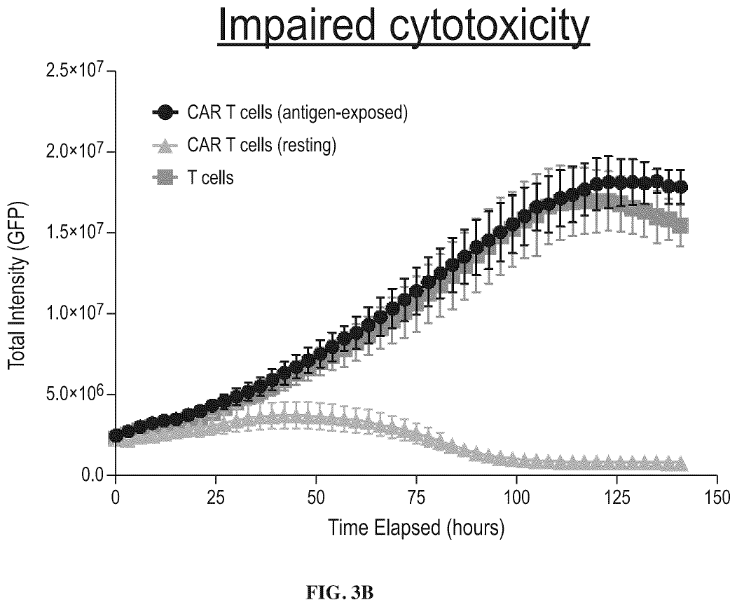

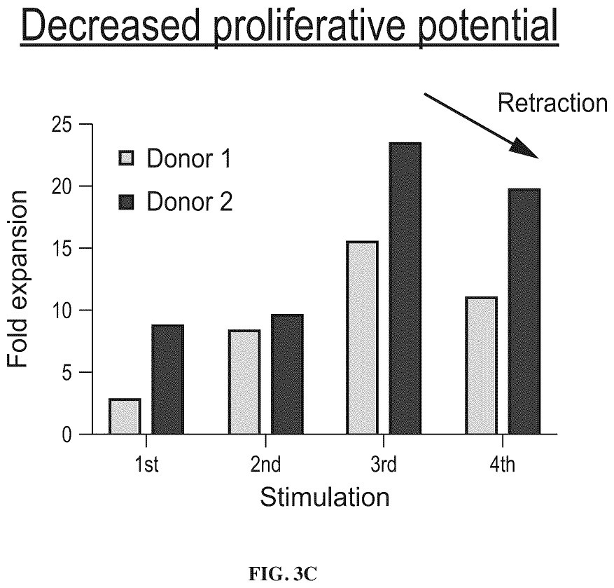

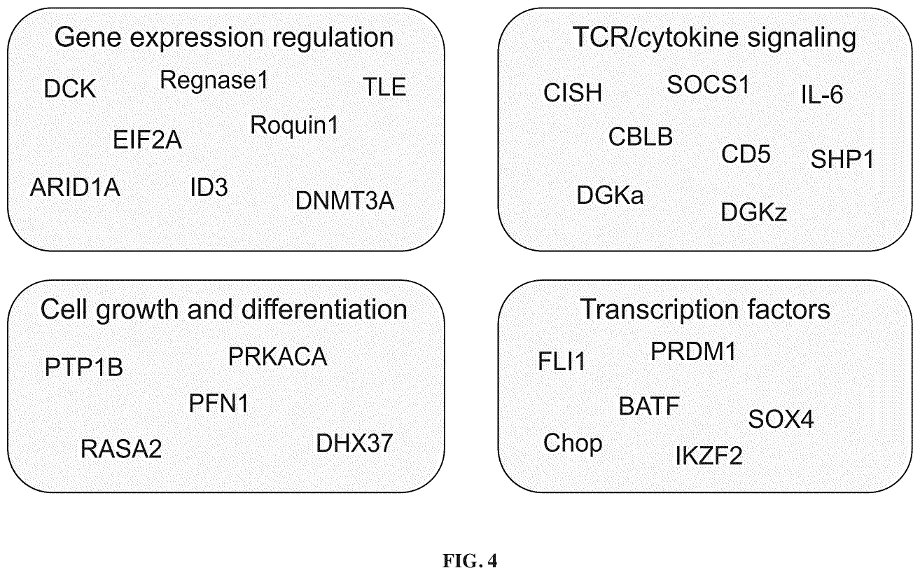

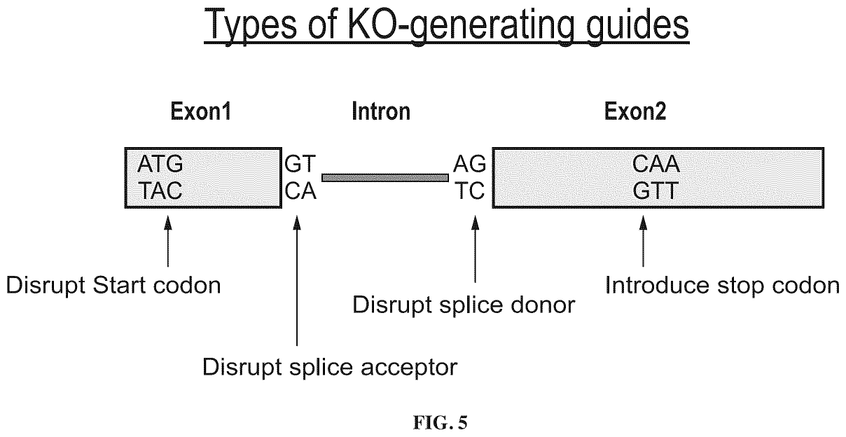

Base editing can be used to modify chimeric antigen receptor expressing T cells (CAR-T cells) to improve their function (see, e.g., FIG. 1). Genetic alterations that improve the ability of the CAR-T cells to function in treating a disease can be referred to as “efficacy edits.” Non-limiting examples of CAR-T cell functions that can be improved through efficacy edits include, but are not limited to, reduced cost of production, reduced or eliminated lymphodepletion, improved function in treatment of solid tumors, improved function in treatment of a disease (e.g., an autoimmune disease), improved potency so that only a small number (e.g., one) doses is necessary for treatment of a disease, and reduced toxicity. Persistent antigen stimulation of CAR-T cells (see, e.g., cells repeatedly exposed to antigen as shown in FIG. 3A) can lead to T cell dysfunction, also referred to as T cells in an “exhausted” or “functionally exhausted” state, characterized by one or more of the following: reduced cytotoxicity (FIG. 3B), reduced proliferation (FIG. 3C), reduced cytokine production, increase in inhibitory receptors, reduced survival, and/or upregulation of repressor genes relative to resting CAR-T cells that were not repeatedly or continuously exposed to antigen. Accordingly, experiments were undertaken to identify efficacy edits carried out using base editing that were effective in reducing levels of T cell dysfunction/exhaustion after repeated or continuous exposure to and stimulation by an antigen. In particular, base editing was used to reduce or eliminate expression of each of the proteins listed in Table 8 below (see also FIG. 4) and the ability of the cells to resist dysfunction resulting from multiple antigen exposures was then evaluated. The proteins were negative regulators of T cell function and functioned in processes such as, to name a few, T cell receptor (TCR) and/or cytokine signaling, gene expression regulation, or cell growth and differentiation, or functioned as transcription factors (FIG. 4).

TABLE 8
List of proteins whose expression was reduced or eliminated through base editing
in CAR-T cells to evaluate the impact of the reduced or eliminated expression
on CAR-T cell function following repeated antigen exposures. For select gene
targets, the guide(s) used in the Examples to target the gene for base editing,
unless indicated otherwise, is indicated under “More info.”

| Protein | Gene | Type | More info. |
|---|---|---|---|
| ARID1A | ARID1A | | Guide: EF117 |
| BATF | BATF | | Guide: EF114 |
| CBLB | CBLB | E3 ubiquitin-protein | Guide: EF2 |
| | | ligase which promotes | |
| | | protease-mediated | |
| | | protein degradation | |
| CD5 | CD5 | Cluster of | Guide: EF94 |
| | | differentiation 5 | |
| Chop | DDIT3 | Downstream sensor of | Guide: EF24. |
| | | ER stress | |
| CISH | CISH | Cytokine Inducible | Guide: EF1 |
| | | SH2 Containing | |
| | | Protein | |
| DCK | DCK | Nucleoside protein | Nucleoside protein |
| | | kinase | kinase. Guide: EF46 |
| DGKα/ζ | DGKα/ζ | Diacylglycerol kinase | Inhibitor in T-cell signal |
| | | | transduction. Guide for |
| | | | DGKa: EF51. Guide for |
| | | | DGKz: EF54. |
| DHX37 | DHX37 | DEAH-Box Helicase | ATP-binding RNA |
| | | 37 | helicase. Guide: EF101. |
| DNMT3A | DNMT3A | Epigenetic regulator | Responsible for de novo |
| | | | methylation. Guide: |
| | | | EF15. |
| EIF2A | EIF2A | Eukaryotic translation | Guide: EF70 |
| | | initiation factor | |
| FLI-1 | FLI-1 | ETS family | Guide: EF20 |
| | | transcription factor | |
| ID3 | ID3 | DNA-binding protein | Guide: EF74 |
| | | inhibitor | |
| IKZF2 | IKZF2 | Member of Ikaros | Upregulated in exhausted |
| | | transcription regulator | T cells. Guide: EE76 |
| | | family (aka Helios) | |
| IL-6 | IL-6 | Interleukin 6 | Cytokine. Guide: EF105 |
| | | | or EF106 |
| PFN1 | PFN1 | Profilin 1 | Regulates |
| | | | actin polymerization. |
| | | | Guide: EF112. |
| PRDM1 | PRDM1 | PR/SET Domain 1 | Repressor of IFNb |
| | | (aka BLIMP-1) | expression. Guide: EF58. |
| PRKACA | PRKACA | Protein Kinase | cAMP-dependent protein |
| | | CAMP-Activated | kinase. Guide: EF62. |
| | | Catalytic Subunit | |
| | | Alpha | |
| PTP1B | PTPN1 | Protein tyrosine | Important in metabolism, |
| | | phosphatase | glucose homeostasis |
| PTPN6 | PTPN6 | Protein Tyrosine | Guide: EF65. |
| | | Phosphatase Non- | |
| | | Receptor Type 6 (aka | |
| | | SHP-1) | |
| RASA2 | RASA2 | RAS P21 Protein | Inhibitory regulator of the |
| | | Activator 2 | Ras-cyclic AMP |
| | | | pathway. Guide: EF96. |
| Regnase-1 | ZC3H12A | Zinc Finger CCCH- | Transcriptional activator. |
| | | Type Containing 12A | Guide: EF25 and/or |
| | | | EF26. |
| Roquin-1 | R3CH1 | Ring Finger And | Recognizes and binds to a |
| | | CCCH-Type Domains | constitutive decay |
| | | 1 | element (CDE) in the 3′ |
| | | | UTR of mRNAs, leading |
| | | | to mRNA deadenylation |
| | | | and degradation. Guide: |
| | | | EF8 |
| SOCS1 | SOCS1 | Suppressor Of | Guide: EF7 |
| | | Cytokine Signaling 1 | |
| SOX4 | SOX4 | Transcription factor | Guide: EF85 |
| | | involved in cell fate | |
| | | determination | |
| TLE | TLE | Repressor of signaling | Transcriptional |
| | | pathways via | corepressor of multiple |
| | | transcription factors | genes encoding |
| | | | inflammatory cytokines. |
| | | | Guide for TLE4: EF86 |
| TMEM184B | TMEM184B | Transmembrane | Guide: EF91 |
| | | protein | |
| TMEM222 | TMEM222 | Transmembrane | Guide: EF107, EF108, |
| | | Protein 222 | EF109, or EF110 |

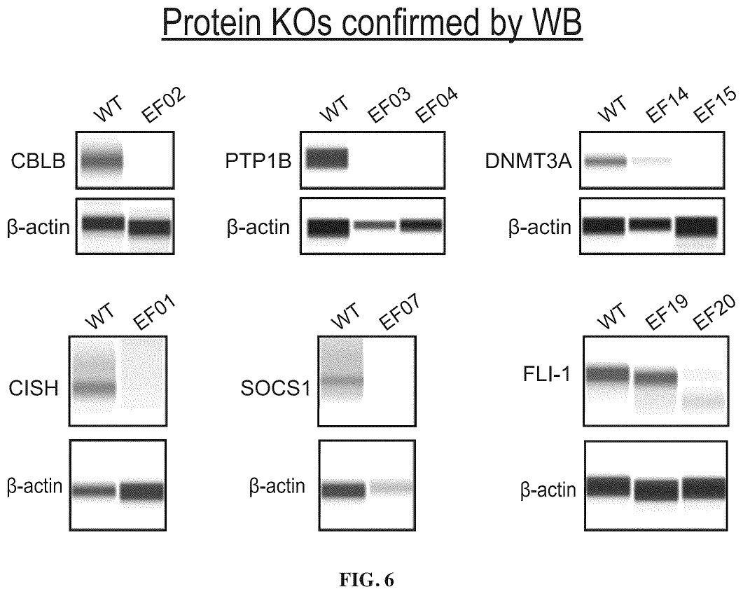

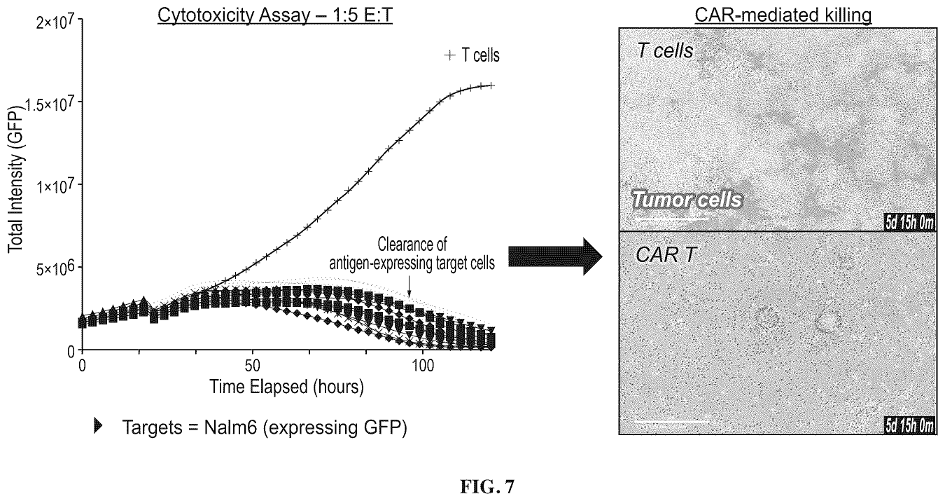

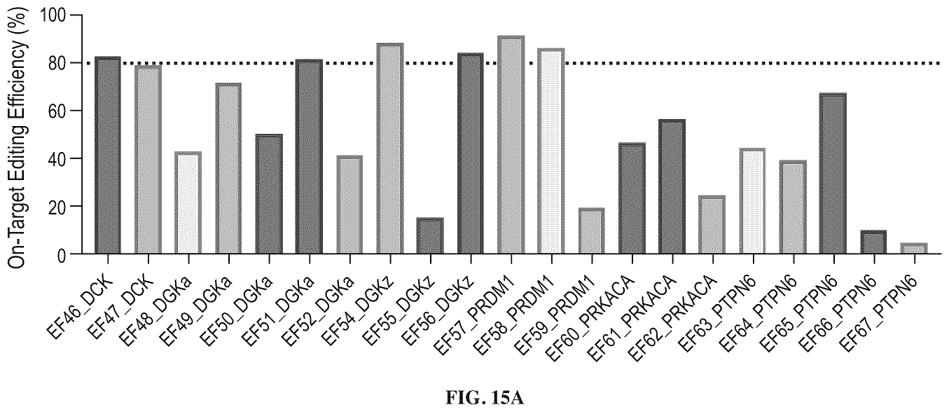

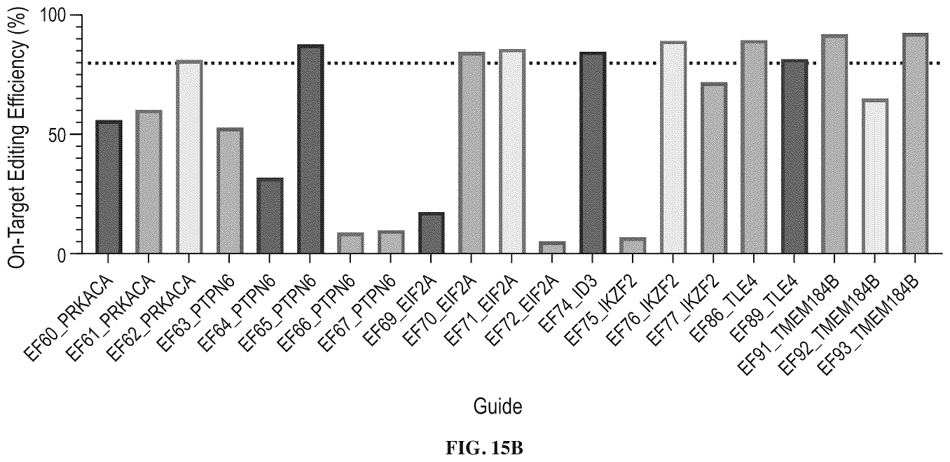

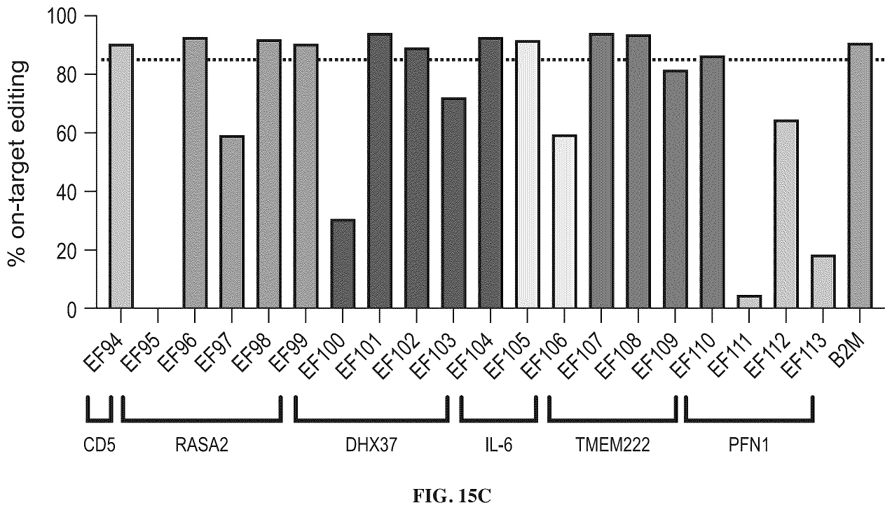

To evaluate the impact of reduced or eliminated expression of each of the polypeptides listed in Table 8 on CAR-T cell resistance to exhaustion after repeated or continuous stimulation by antigen exposures, base editing was used to introduce nucleotide alterations to polynucleotides encoding the polypeptides in CAR-T cells that led to a reduction or elimination in expression of the polypeptides. All of the CAR-T cells expressed an anti-CD19 chimeric antigen receptor containing the following domains, from N-terminus to C-terminus: an anti-CD19 scFv antigen binding domain, a CD8-derived hinge domain, a CD8-derived transmembrane domain, a 4-1BB costimulatory domain, and a CD3ζ domain. The chimeric antigen receptors were fused at the C-terminus by way of a T2A self-cleaving peptide to an epidermal growth factor receptor (EGFR) derived tag. The amino acid sequence of the anti-CD19 chimeric antigen receptor is provided below and in the Sequence Listing as SEQ ID NO: 830. The chimeric antigen receptor (CAR) was introduced to the T cells by transducing them using a lentiviral particle containing a polynucleotide encoding the CAR. The CAR-T cells were each prepared using T cells collected from a human donor subject. The CAR-T cells were base edited using base editor systems containing guide polynucleotides containing spacers and/or sgRNA sequences listed in Tables 1, 2A, and/or 2B in combination with one of the suitable base editors listed in Table 2B and corresponding to the spacer of the guide polynucleotide. Unless indicated otherwise, cells referenced in the figures of the application were base edited using the base editor ABE8.20m in combination with a guide polynucleotide containing a sequence corresponding to a sequence listed in Table 20 above. The base editor systems were introduced into the T cells by contacting the cells with the guide polynucleotide and an mRNA molecule encoding the base editor (ABE8.20m). The base editor systems introduced base edits to polynucleotides encoding each of the polypeptides listed in Table 8 that disrupted expression of the polypeptides therefrom by either 1) disrupting a start codon, 2) disrupting a splice acceptor (SA) site, 3) disrupting a splice donor (SD) site, 4) or introducing a stop codon (see FIG. 5). The nucleobases targeted for base editing were contained within about the first 50% of the nucleotides of each polynucleotide corresponding to the mRNA molecule transcribed therefrom and encoding the polypeptide. The base editing window for each base editor system was from about 4 to about 7 nucleotides. Western blots were used to confirm that base editing resulted in reduced or eliminated expression of the polypeptides. For example, Western Blots were used to confirm that cells base edited using one of the following guide polynucleotides (see Tables 1 and 2A for corresponding sequences) showed reduced or undetectable expression of the polypeptides (e.g., CBLB, PTP1B, DNMT3A, CISH, SOCS1, and FLI-1) encoded by polynucleotides targeted with the following guides, whose sequences are provided at Tables 1 and 2A: EF46, EF47, EF48, EF49, EF50, EF51, EF52, EF53, EF54, EF55, EF56, EF57, EF58, EF59, EF60, EF61, EF62, EF63, EF64, EF65, EF66, EF67, EF68, EF69, EF70, EF71, EF72, EF73, EF74, EF75, EF76, EF77, EF86, EF87, EF88, EF89, EF90, EF91, EF92, EF93, EF94, EF95, EF96, EF97, EF98, EF99, EF100, EF101, EF102, EF103, EF104, EF105, EF106, EF107, EF108, EF109, EF110, EF111, EF112, and EF113 (FIG. 6). Next-generation sequencing was used to evaluate percent on-target editing efficiencies for many of the base editor systems (FIGS. 15A-15C), and many of the base editor systems, including those containing one of the following guide polynucleotides (see Tables 1 and 2A for corresponding sequences), had percent on-target editing efficiencies of over about 80%: EF1, EF2, EF3, EF4, EF7, EF8, EF12, EF14, EF15, EF19, EF20, EF24, EF25, EF26, EF46, EF47, EF51, EF54, EF56, EF57, EF58, EF62, EF65, EF70, EF71, EF74, EF76, EF86, EF89, EF91, EF93, EF94, EF96, EF98, EF99, EF101, EF102, EF104, EF105, EF107, EF108, and EF110.

An experiment was undertaken to determine whether cells base edited to reduce or eliminate expression of one of the following polypeptides: CISH, CBLB, PTP1B, SOCS1, Roquin-1, DNMT3A, FLI-1, Chop, and Regnase-1 still had cytotoxic capacity. To evaluate the cytotoxic capacity of the base-edited CAR-T cells, the cells were co-cultured with target cells (Nalm6 B cell precursor leukemia cells initiated from an adolescent male and that expressed green fluorescent protein (GFP)) at an effector to target cell ratio (E:T) of 1:5, and target cell proliferation was monitored over time by measuring GFP fluorescence/expression. Interestingly, the base-edited CAR-T cells maintained the ability to kill the target cells, as shown by clearance of the antigen-expressing target cells from the co-culture (FIG. 7).

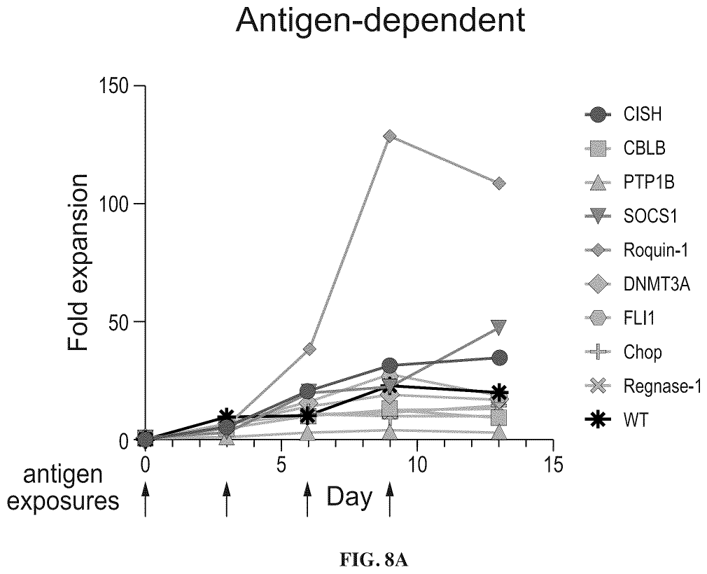

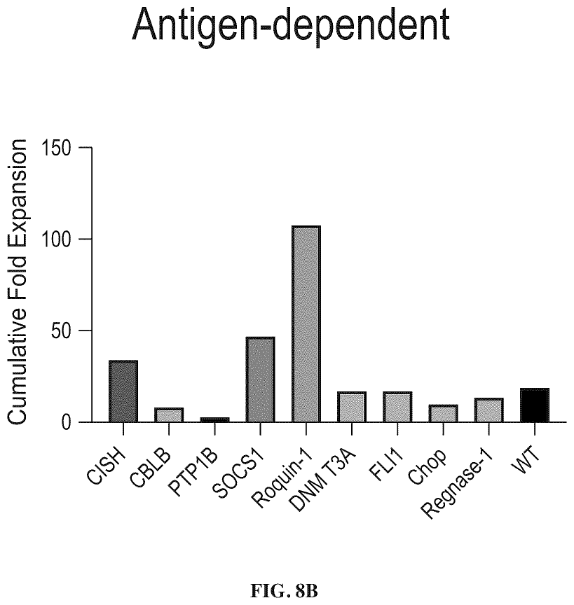

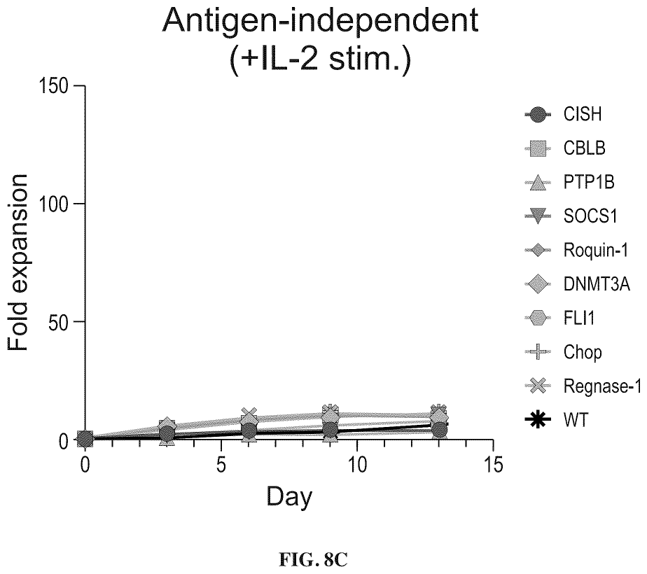

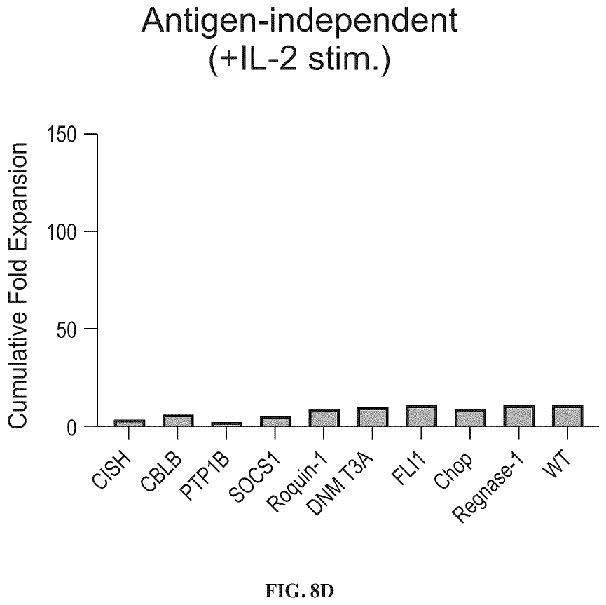

An experiment was undertaken to show that the base-edited CAR-T cells showed improved antigen-dependent expansion/proliferation relative to unedited CAR-T cells. CAR-T cells base edited to reduce or eliminate expression of CISH, CBLB, PTP1B, SOCS1, Roquin-1, DNMT3A, FLI-1, Chop, or Regnase-1 were either exposed to target antigen (CD19) (antigen-dependent expansion) at days 0, 3, and 9 or never exposed to antigen (antigen-independent expansion) and fold expansion was monitored from day 0 to day 15. It was determined that CAR-T cells base edited to reduce or eliminate expression of CISH, SOCS1, or Roquin-1 showed increased antigen-dependent expansion relative to unedited CAR-T cells (FIGS. 8A-8D).

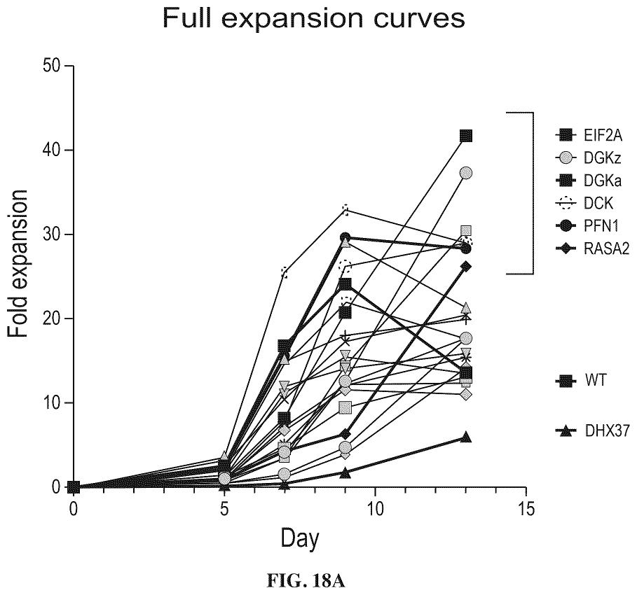

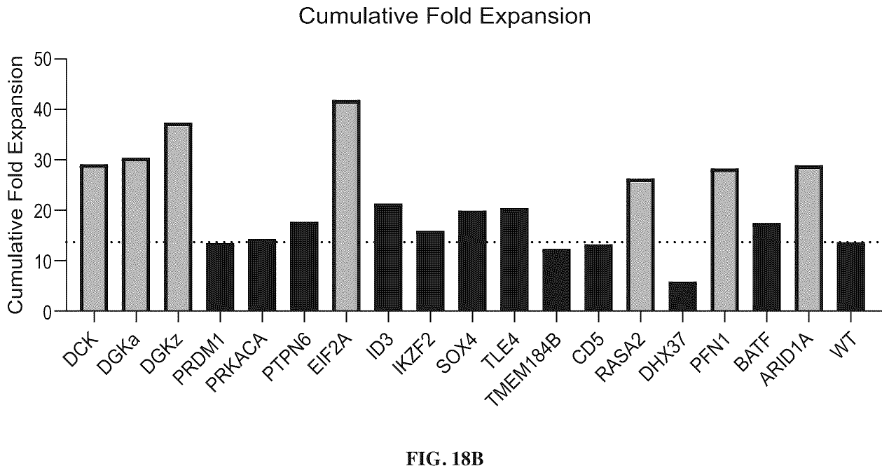

An experiment was next undertaken to evaluate whether the base-edited CAR-T cells showed improved proliferation relative to unedited CAR-T cells after multiple antigen exposures. CAR-T cells base edited to reduce or eliminate expression of DCK, DGKa, DGKz, PRDM1 (BLIMP-1), PRKACA, PTPN6, EIF2A, ID3, IKZF2, SOX4, TLE4, TMEM184B, CD5, RASA2, DHX37, PFN1, BATF, or ARID1A were stimulated multiple times (e.g., four times for the data shown in FIG. 18A) by being exposed to antigen (see FIG. 3A) and then co-cultured overnight with Nalm6 cells (B cell precursor leukemia cells initiated from an adolescent male) at an effector to target cell (E:T) ratio of, for example, 1:1 in FIG. 18B. All of the CAR-T cells were prepared using T cells collected from the same donor subject. CAR-T cells base edited to reduce or eliminate expression of DCK, DGKa, DGKz, EIF2A, RASA2, PFN1, or ARID1A showed enhanced proliferation after the four antigen exposures relative to unedited CAR-T cells (FIGS. 18A and 18B). This indicates that base editing to reduce expression of DCK, DGKa, DGKz, EIF2A, RASA2, PFN1, or ARID1A polypeptides was effective in overcoming exhaustion related reductions in proliferation.

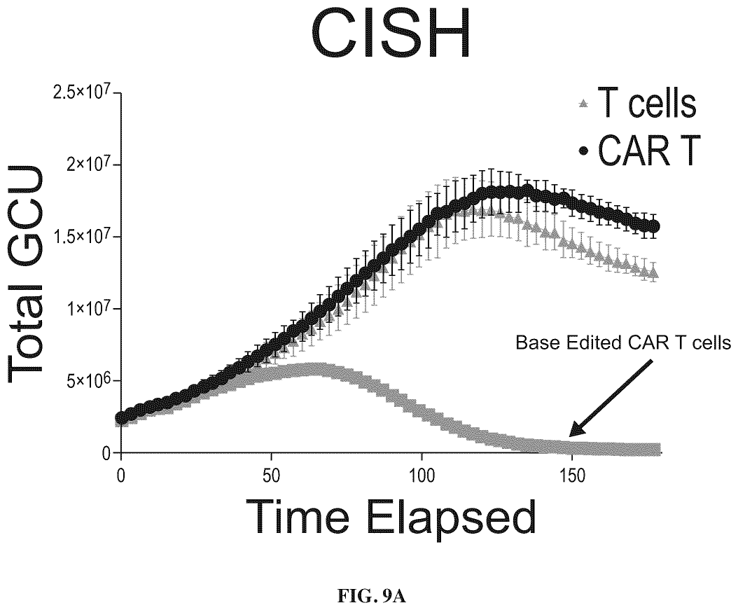

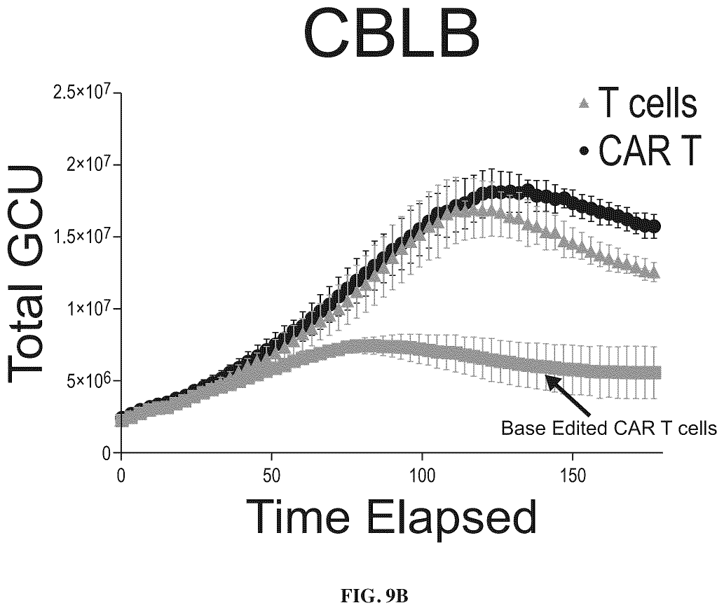

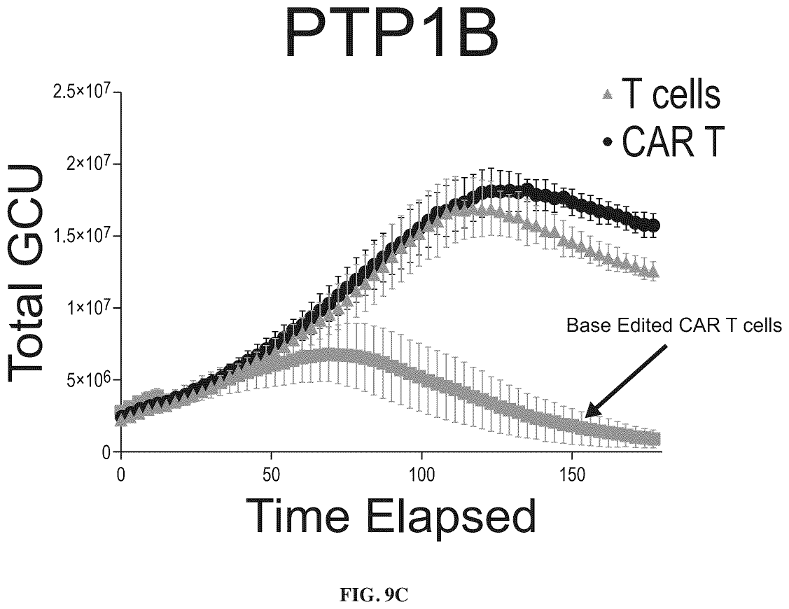

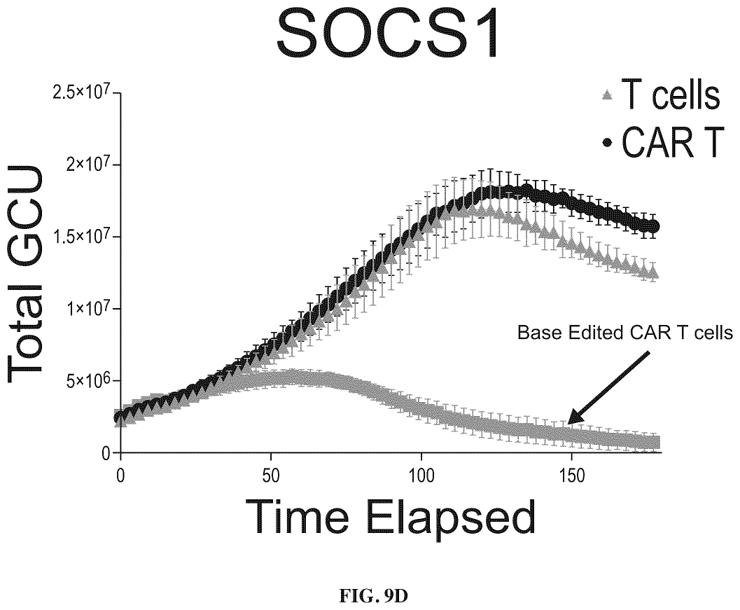

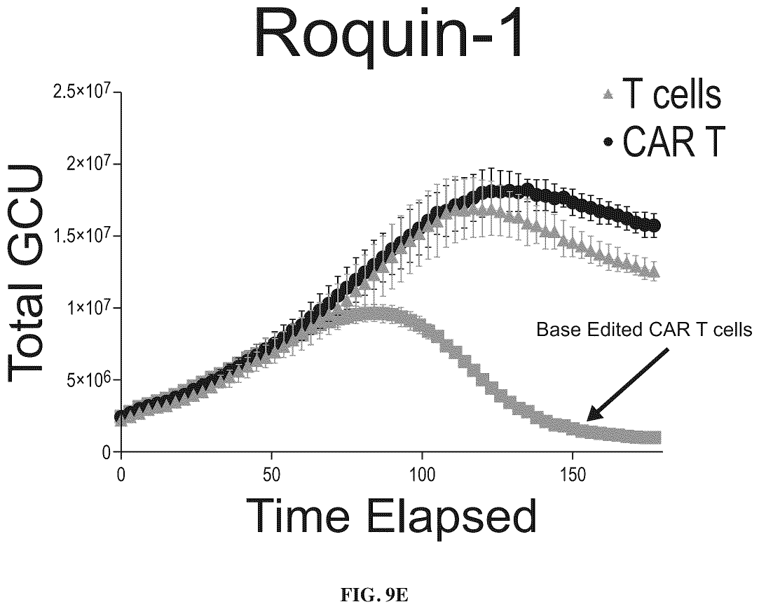

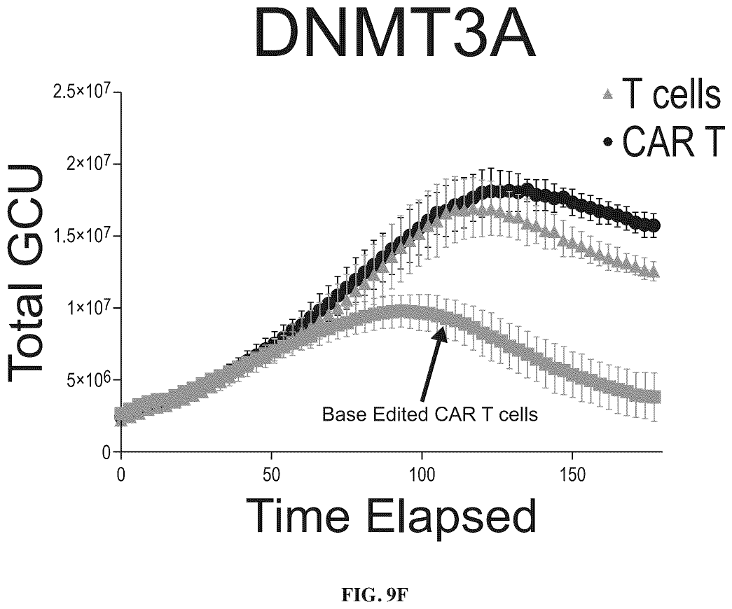

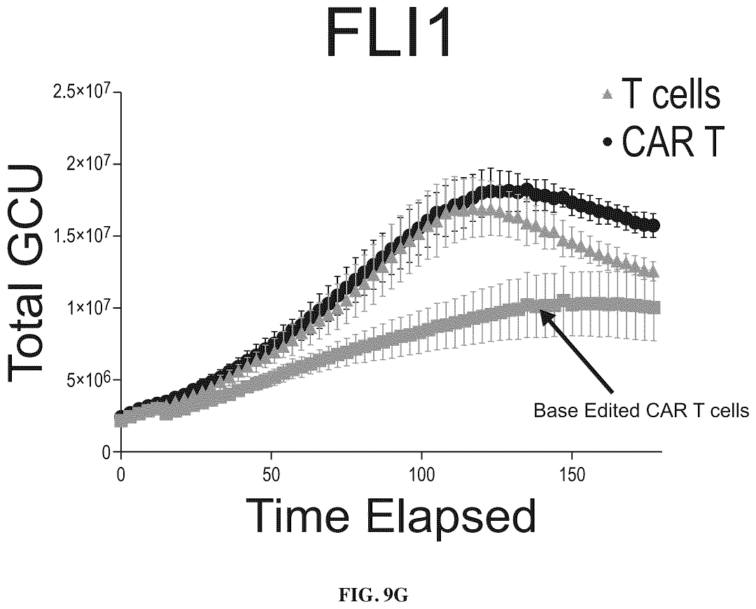

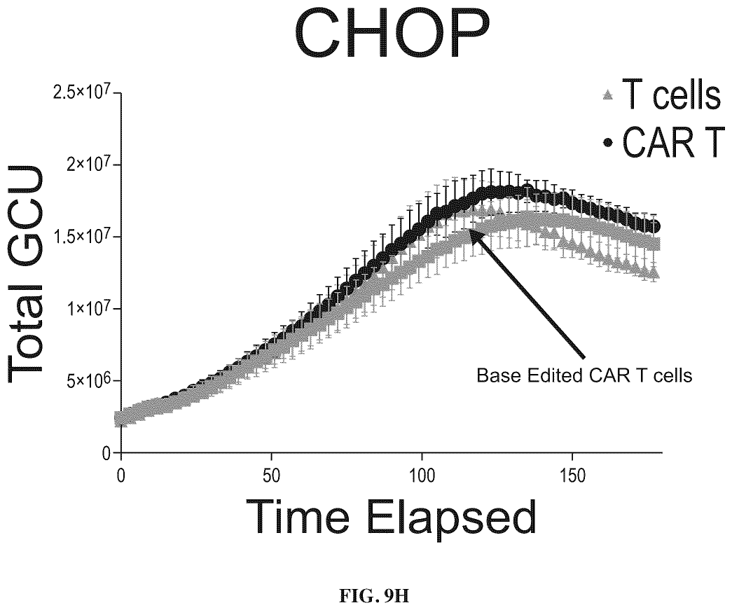

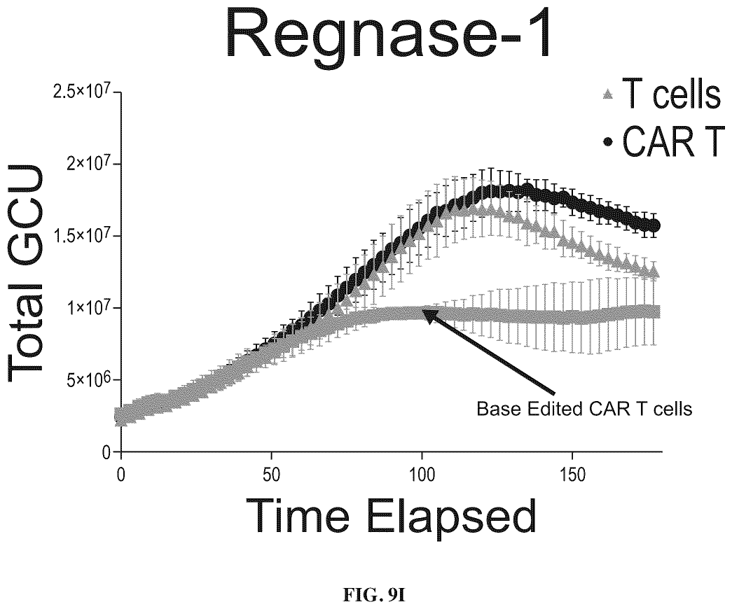

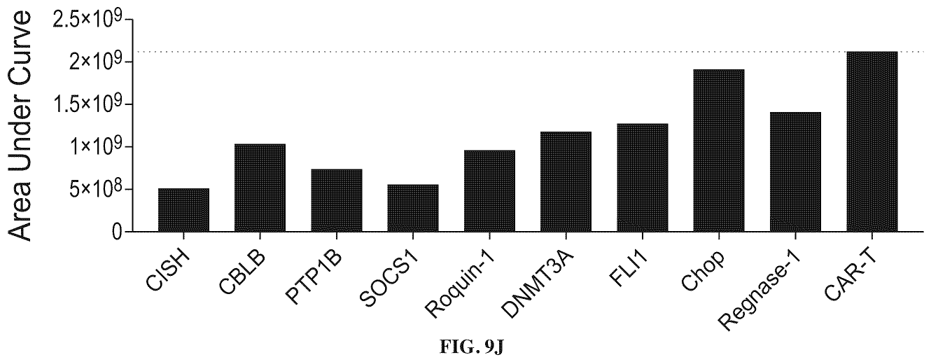

An experiment was then undertaken to determine whether CAR-T cells base edited to reduce or eliminate expression of CISH, CBLB, PTP1B, SOCS1, Roquin-1, DNMT3A, FLI-1, Chop, or Regnase-1 polypeptides showed reduced levels of dysfunction relative to unedited CAR-T cells after repeated antigen exposures. The CAR-T cells were exposed to target antigen four times, as shown in FIG. 3A, and then co-cultured with Nalm6 (B cell precursor leukemia cells initiated from an adolescent male) that expressed green fluorescent protein (GFP) at an effector to target cell (E:T) ratio of 1:5. Clearance of the Nalm6 target cells was evaluated by measuring GFP fluorescence in the co-culture over time. All of the base-edited CAR-T cells showed increased capacity to clear the Nalm6 tumor cells from the co-culture relative to the unedited CAR-T cells (FIGS. 9A-9J), thereby showing that the base edited CAR-T cells showed improved resistance to exhaustion associated with repeated antigen stimulations.

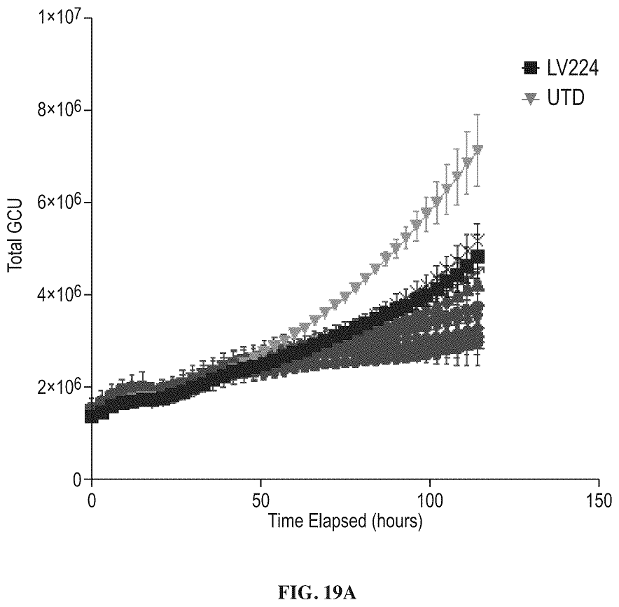

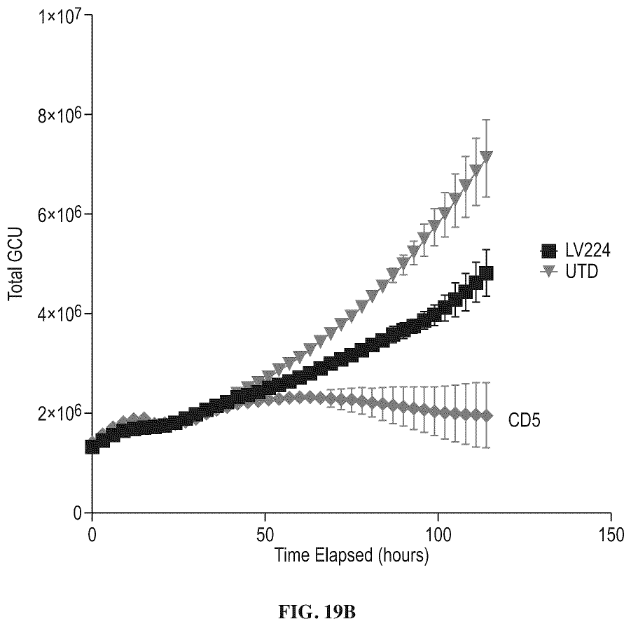

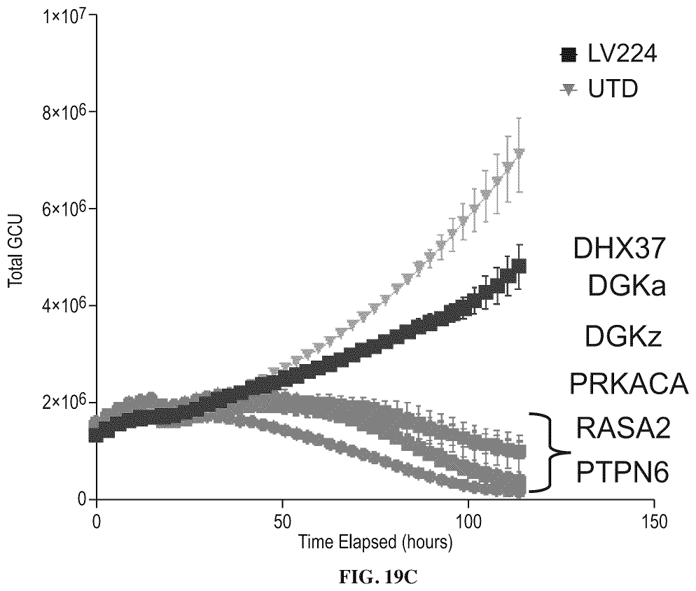

A further experiment was undertaken to determine whether CAR-T cells base edited to reduce or eliminate expression of DCK, CD5, DGKα/ζ (DGKa and DGKz), DHX37, EIF2A, ID3, IKZF2, IL-6, PFN1, PRDM1, PRKACA, PTPN6, RASA2, SOX4, TLE, TMEM184B, or TMEM222 showed reduced levels of exhaustion, measured as increased cytotoxicity, relative to unedited CAR-T cells after repeated antigen exposures. Prior to measuring cytotoxicity, the CAR-T cells were stimulated four times by being exposed to CD19 antigen (see FIG. 3A). To measure cytotoxicity, the cells were co-cultured overnight with Nalm6 cells (B cell precursor leukemia cells initiated from an adolescent male) expressing green fluorescent protein (GFP) at an effector to target cell (E:T) ratio of 1:5. Clearance of the Nalm6 cells was measured over time using live cell fluorescent imaging carried out using an IncuCyte® Live-Cell Analysis System. Many of the base-edited CAR-T cells showed increased cytotoxicity (i.e., improved resistance to exhaustion) after multiple antigen exposures relative to unedited CAR-T cells (FIGS. 19A-19C).

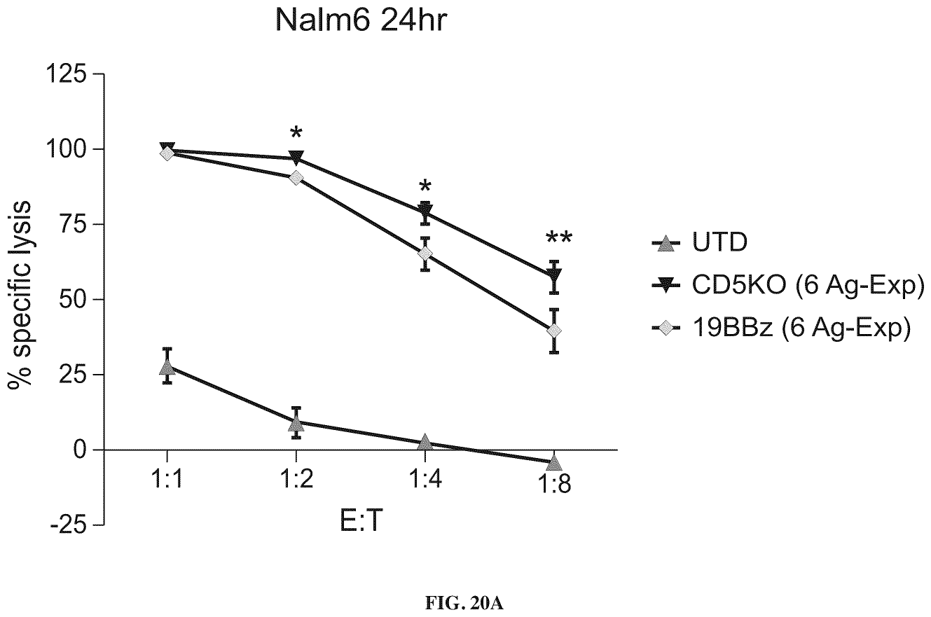

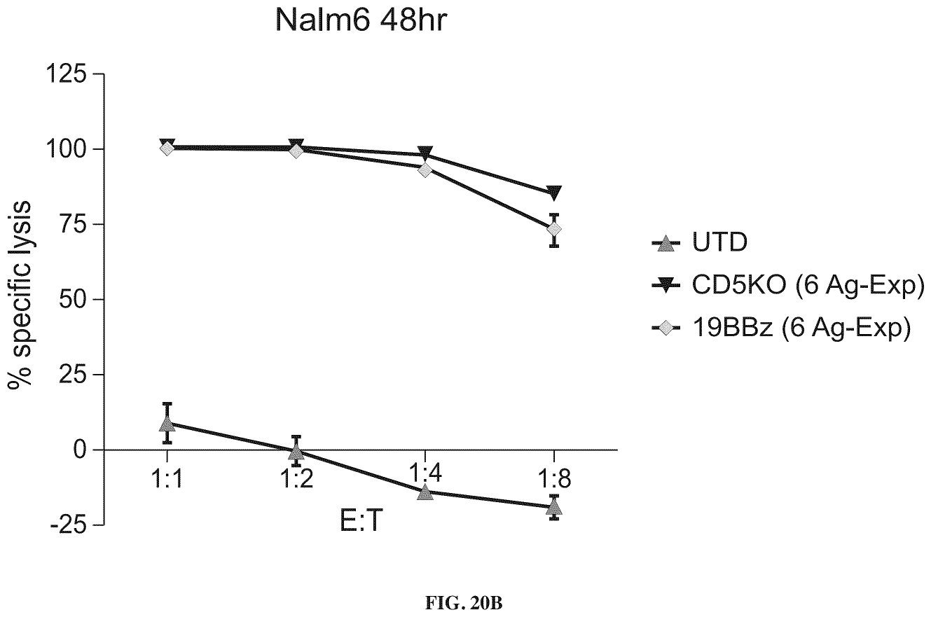

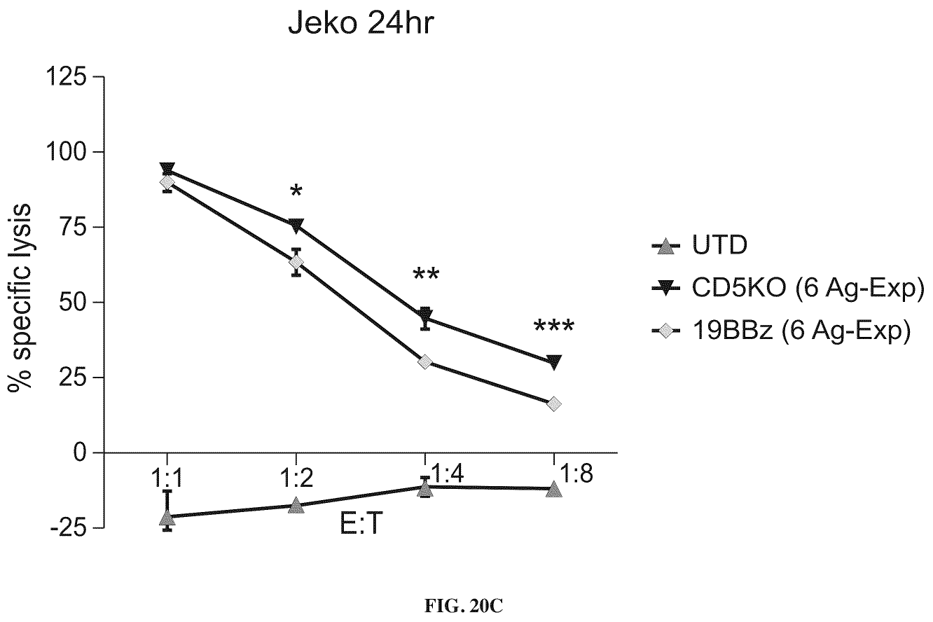

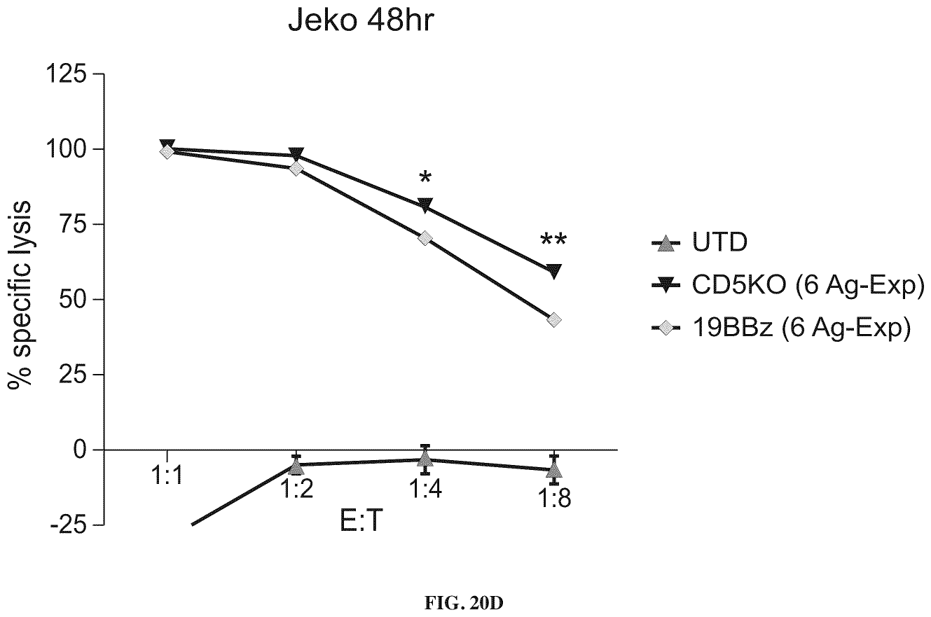

A similar experiment was then undertaken to determine whether chimeric antigen receptor (CAR) T cells base edited to reduce or eliminate expression of CD5 showed reduced levels of dysfunction, measured as increased cytotoxicity, relative to unedited CAR-T cells after repeated antigen exposures. The CAR-T cells were co-cultured with target cells at effector to target cell ratios (E:T) of 1:1, 1:2, 1:4, and 1:8, and lysis/clearance of the target cells was evaluated after 24 hours and 48 hours of coculture. Prior to being co-cultured with target cells, the T cells were exposed to CD19 antigen six (6) times (see FIG. 3A). The target cells were either JeKo-1 cells (a mantle cell lymphoma (MCL) cell line isolated from the peripheral blood mononuclear cells of a 78-year-old female with a large cell variant of MCL showing leukemic conversion) or Nalm6 cells (B cell precursor leukemia cells initiated from an adolescent male). The base-edited CAR-T cells showed increased cytotoxicity against both the JeKo-1 and the Nalm6 target cells at all evaluated E:T ratios after repeated exposures to the target antigen (a CD19 antigen) relative to unedited CAR-T cells (FIGS. 20A-20D).

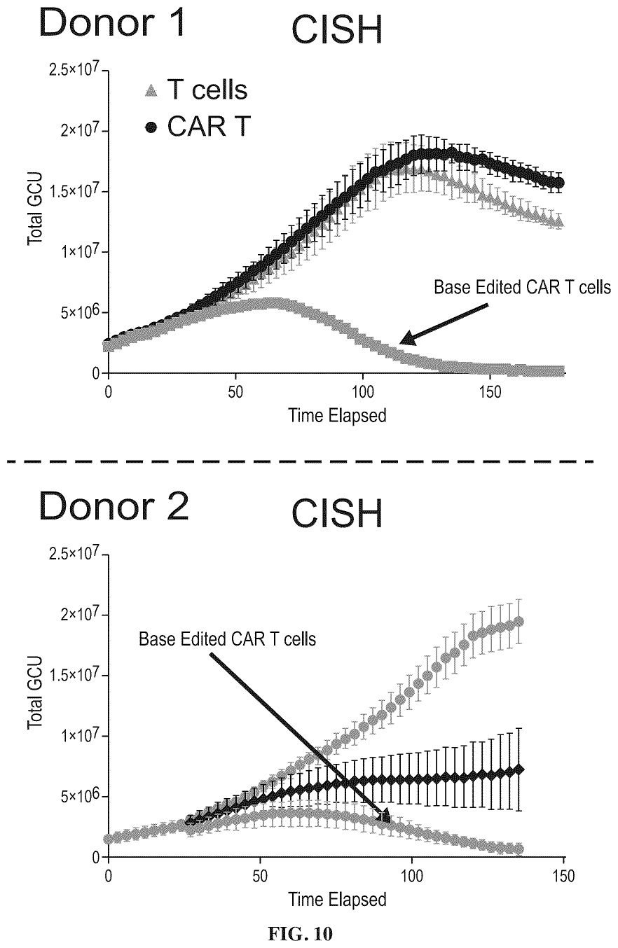

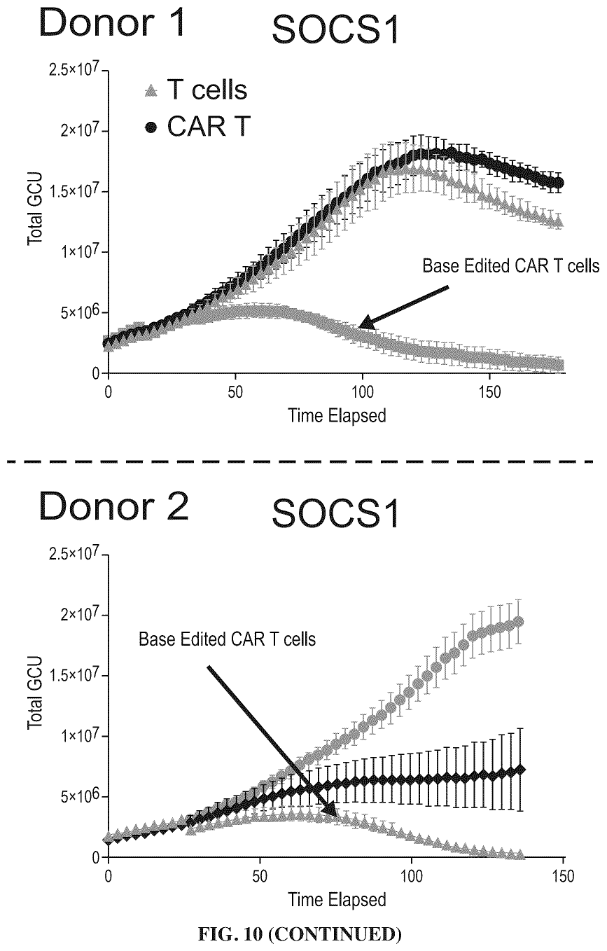

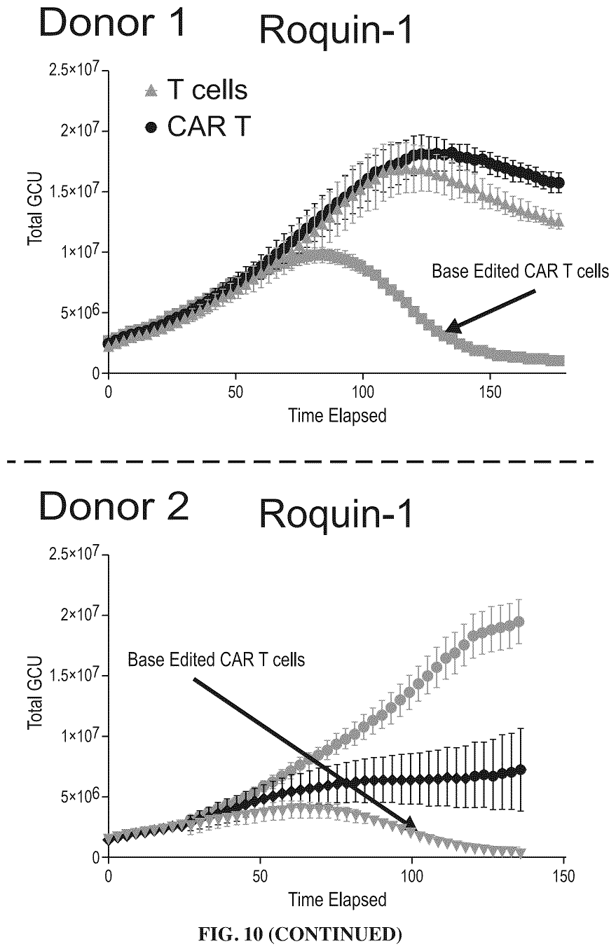

The observed improvement in resistance to dysfunction associated with repeated antigen stimulations observed in the base-edited CAR-T cells was observed across donors. Anti-CD19 CAR-T cells prepared from two different human donor subjects (Donor 1 and Donor 2) were base-edited as described above to reduce or eliminate expression of CISH, SOCS1, or Roquin-1. The CAR-T cells were exposed to target antigen 4 times, as shown in FIG. 3A, and then co-cultured with Nalm6 (B cell precursor leukemia cells initiated from an adolescent male) that expressed green fluorescent protein (GFP) at an effector to target cell (E:T) ratio of 1:5 and clearance of the Nalm6 target cells was evaluated by measuring GFP fluorescence in the co-culture over time. The base-edited CAR-T cells showed increased capacity to clear the Nalm6 tumor cells from the co-culture relative to the unedited CAR-T cells (FIG. 10), regardless of the donor from which the CAR-T cells were derived. Therefore, the improved resistance to dysfunction was observed in CAR-T cells across different donor subjects (i.e., in T cells derived from different donor subjects).

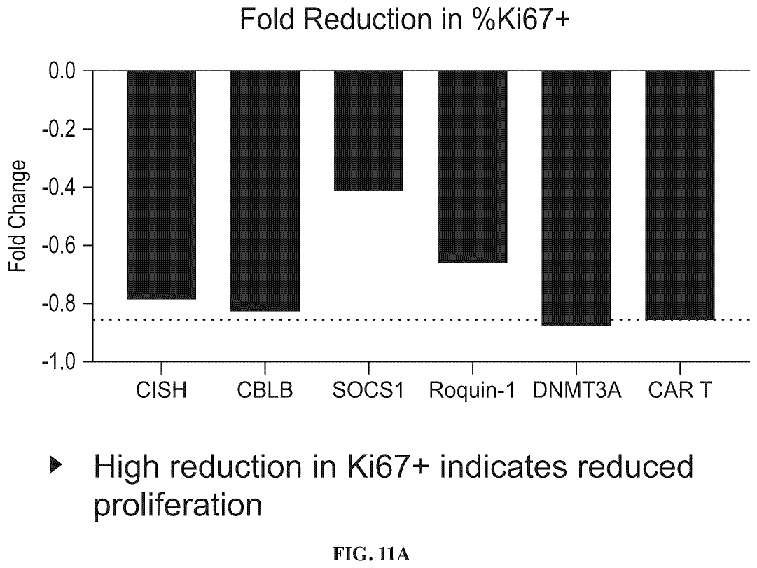

CAR-T cells base edited to reduce or eliminate expression of CISH, CBLB, SOCS1, Roquin-1, or DNMT3A showed improved intrinsic phenotypes after repeated stimulations by a target antigen, relative to unedited CAR-T cells. For example, when the base edited CAR-T cells were exposed to antigen four times (see protocol shown in FIG. 3A), the CAR-T cells showed lower fold reductions in instances of the Ki67+ phenotype and lower levels of the EOMES+ T-bet− phenotype relative to the unedited CAR-T cells (FIGS. 11A and 11B). EOMES+ and T-bet− are factors linked to an exhausted T cell phenotype, and a higher reduction in Ki67+ indicates a higher reduction in proliferation. Therefore, the lower fold reductions (i.e., lower magnitude of negative fold changes) in Ki67+ indicated that the base edited CAR-T cells had increased capacity for proliferation relative to the unedited CAR-T cells, and the reduced instance of the EOMES+T-bet− phenotype in the base-edited CAR-T cells indicated a reduction in occurrences of an exhausted phenotype (e.g., reduced cytotoxicity, reduced proliferation, reduced cytokine production, reduced survival, increase in inhibitory receptors, and/or upregulation of repressor genes) in the base edited CAR-T cells.

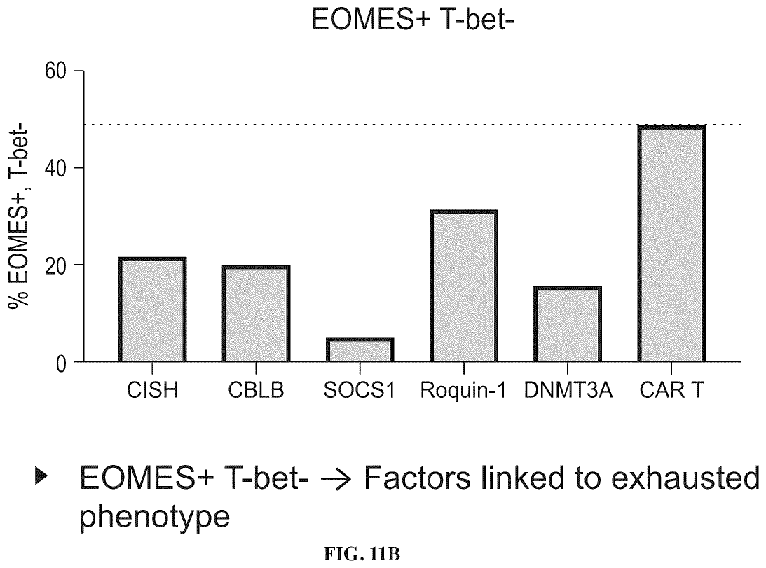

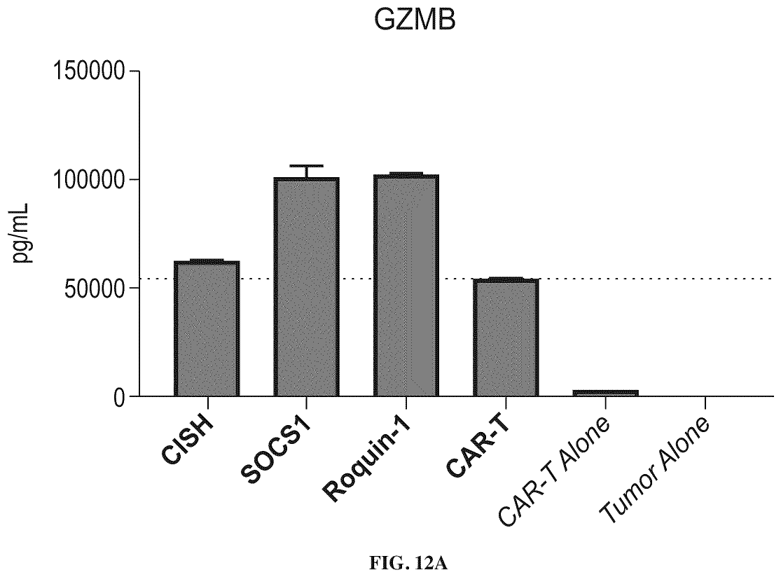

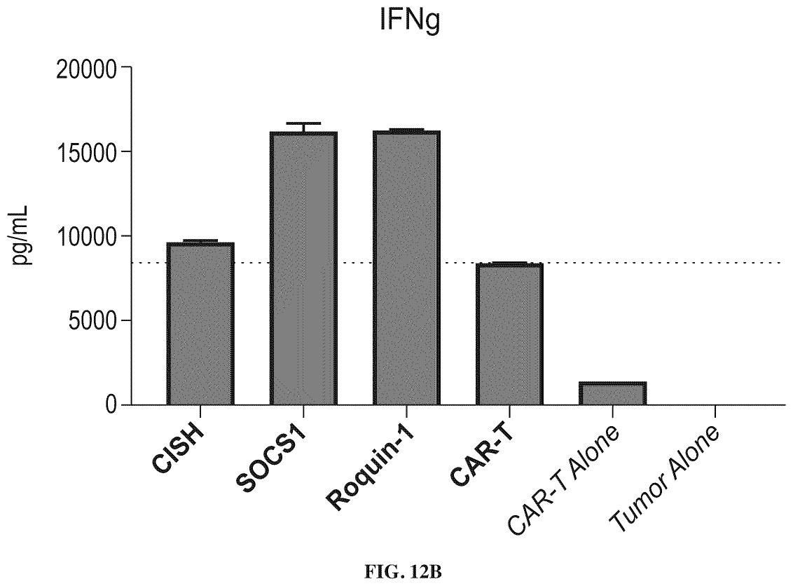

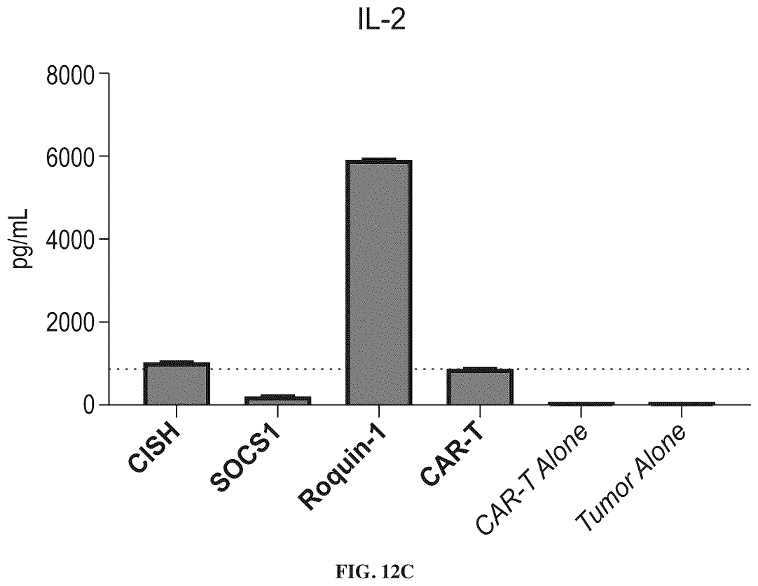

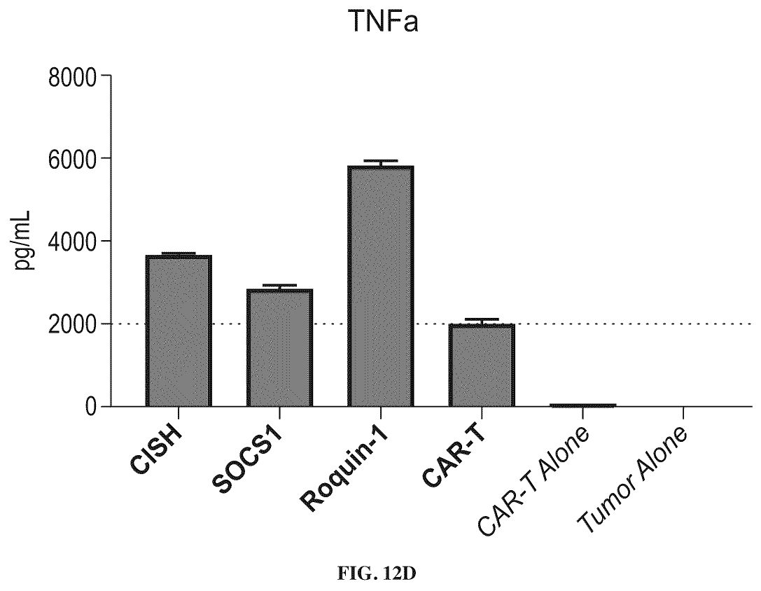

CAR-T cells base edited to reduce or eliminate expression of CISH, SOCS1, or Roquin-1 maintained robust cytokine (e.g., GZMB, IL-2, IFNg, TNFa) secretion after repeated antigen exposures. The CAR-T cells were exposed to antigen four times (see FIG. 3A) prior to co-culturing the cells with target cells and subsequently measuring, in pg/mL, secretion of the cytokines granzyme B (GZMB; FIG. 12A), interferon gamma (IFNg; FIG. 12B), interleukin-2 (IL-2; FIG. 12C), and tumor necrosis factor alpha (TNFa; FIG. 12D). Prior to measuring cytokine secretion, the CAR-T cells were co-cultured with target cells (Nalm6 B cell precursor leukemia cells initiated from an adolescent male and that expressed green fluorescent protein (GFP)) at an effector to target cell ratio (E:T) of 1:1. The base edited CAR-T cells showed increased levels of secretion of most of the cytokines relative to unedited CAR-T cells.

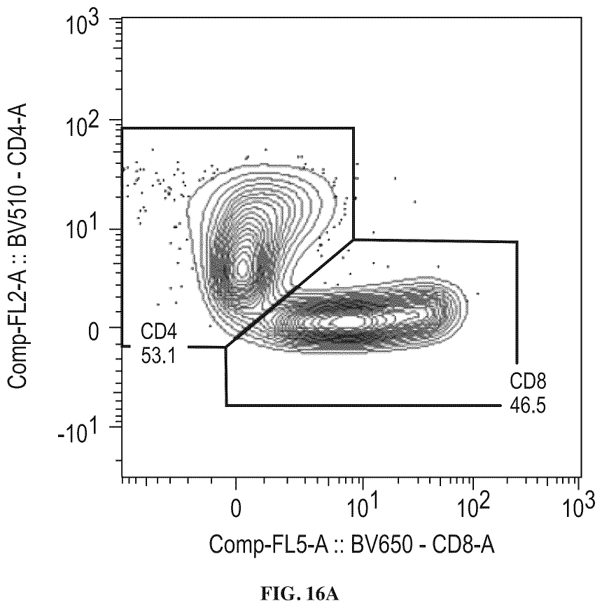

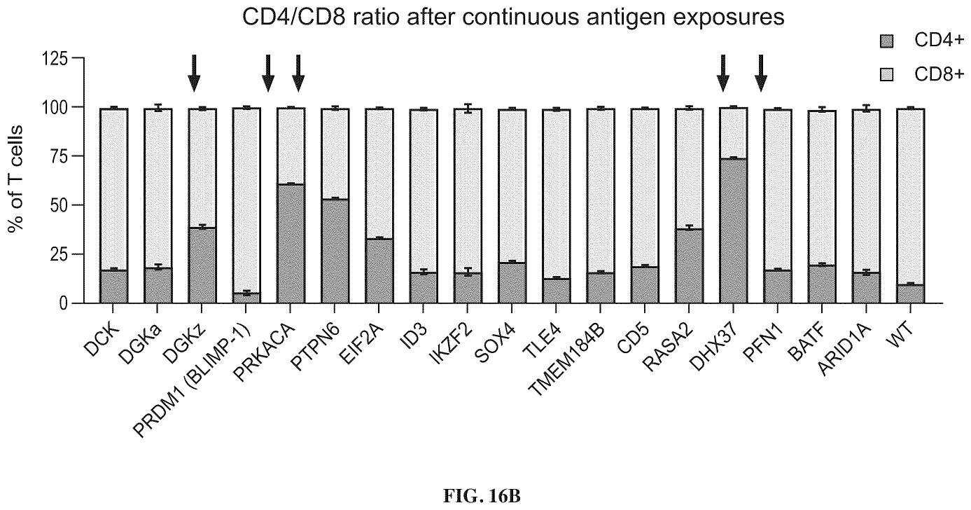

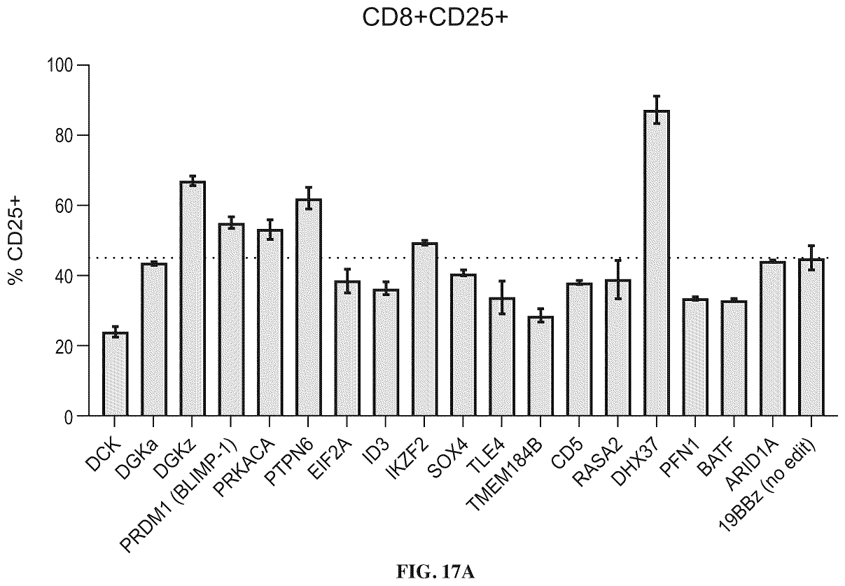

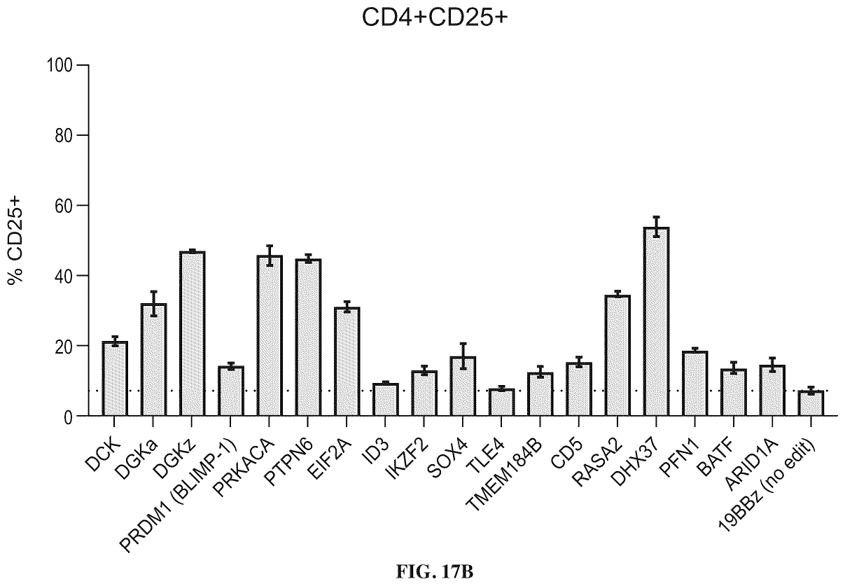

CAR-T cells base edited to reduce or eliminate expression of DCK, DGKa, DGKz, PRDM1 (BLIMP-1), PRKACA, PTPN6, EIF2A, ID3, IKZF2, SOX4, TLE4, TMEM184B, CD5, RASA2, DHX37, PFN1, BATF, or ARID1A had altered CD4+ to CD8+ ratios and enhanced activation by antigen after repeated antigen exposures relative to unedited CAR-T cells. Prior to measuring CD4+ to CD8+ ratios or cell activation using flow cytometry (see, e.g., FIG. 16A), the CAR-T cells were stimulated four (4) times by being exposed to antigen (see FIG. 3A) and then co-cultured overnight with Nalm6 cells (B cell precursor leukemia cells initiated from an adolescent male) at an effector to target cell (E:T) ratio of 1:1. It was found that the base edited CAR-T cells showed altered CD4+ to CD8+ ratios relative to unedited CAR-T cells (FIG. 16B). For example, the CAR-T cells base edited to reduce or eliminate expression of DGKz, PRKACA, PTPN6, RASA2, or DHX37 all showed increased instances of the CD4+ phenotype and reduced instances of the CD8+ phenotype relative to unedited CAR-T cells (FIG. 16B). It was also found that the base edited CAR-T cells having the CD8+ or CD4+ phenotype showed enhanced levels of activation relative to unedited CAR-T cells, where increases in activation was measured as increased instances of the CD25+ phenotype (FIGS. 17A and 17B). These findings confirmed that base edited CAR-T cells showed improved resistance to developing an exhausted phenotype after repeated antigen exposures relative to unedited CAR-T cells.

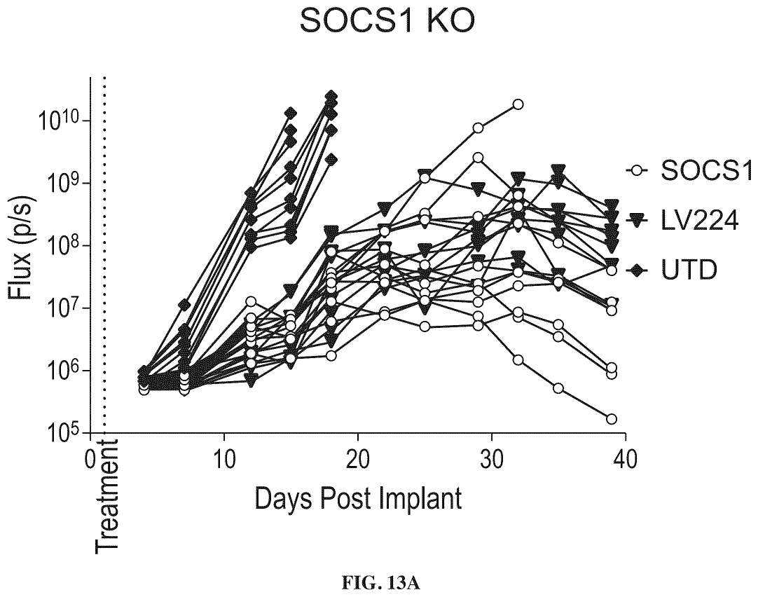

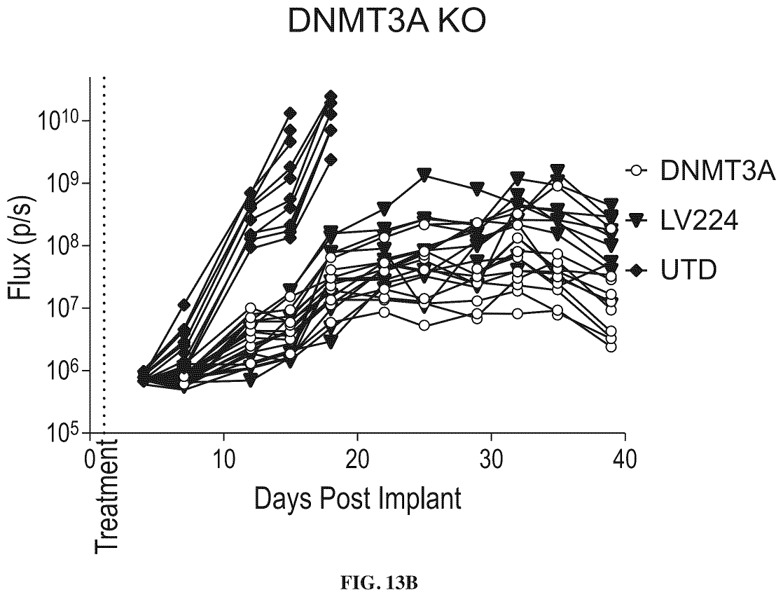

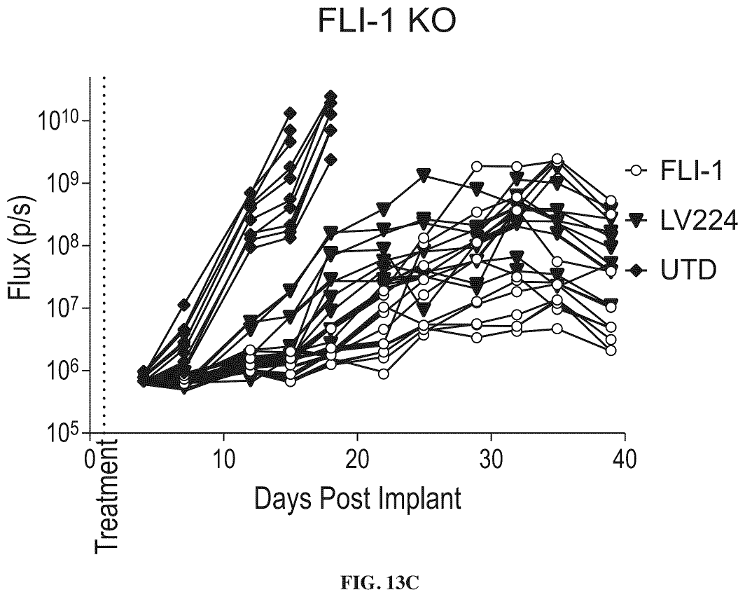

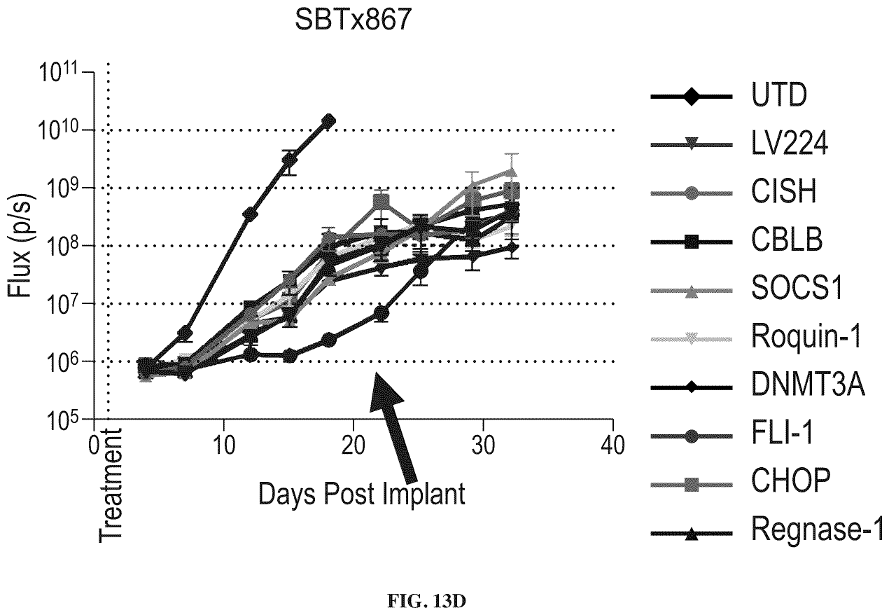

Experiments were next undertaken using two sub-therapeutic models to evaluate the ability of the base edited CAR-T cells to clear tumor cells from a subject in vivo. In a first experiment, anti-tumor activity of CAR-T cells base edited using the base editor ABE8.20m in combination with a guide polynucleotide corresponding to EF01, EF02, EF08, EF15, EF20, EF24, or EF25 (see Tables 1, 2A, and 2B for sequences) to reduce or eliminate expression of CISH, CBLB, SOCS1, Roquin-1, DNMT3A, FLI-1, CHOP, or Regnase-1, respectively, was evaluated using a sub-therapeutic B cell lymphoma (BCL) model involving the intravenous administration of 5E5 Raji cells (a human B lymphoblastoid cell line derived from a patient with Burkitt lymphoma) expressing luciferase into 6-7 week-old NSG female mice (Jackson Labs: Stock 05557) at day zero (0) and subsequently infusing 1E6 of the base edited CAR-T cells (N=10 for each base-edited CAR-T cell and N=10 or 5 for non-base-edited cells) into the mice at day 1. Groups were randomized based on mouse body weight. Tumor clearance was monitored over time (2×/wk) by measuring luciferase flux in photons per second (p/s) in the mice using an IVIS® Spectrum in vivo imaging system. Also, measurements of mouse bodyweights were measured and clinical observations were collected 2×/wk. Several of the base edited CAR-T cells (e.g., cells base edited to reduce or eliminate expression of FLI-1) showed improved antitumor activity in vivo in the BCL model (FIGS. 13A-13D).

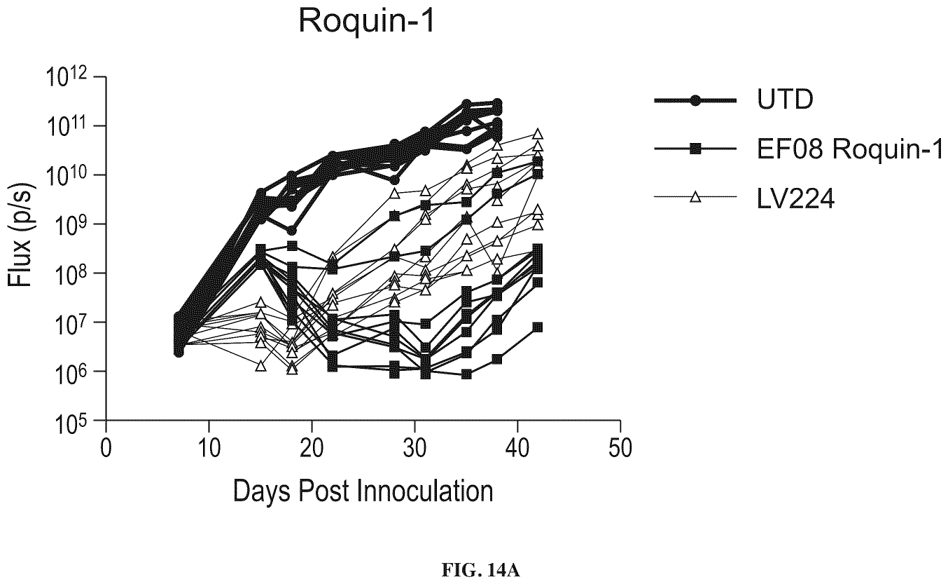

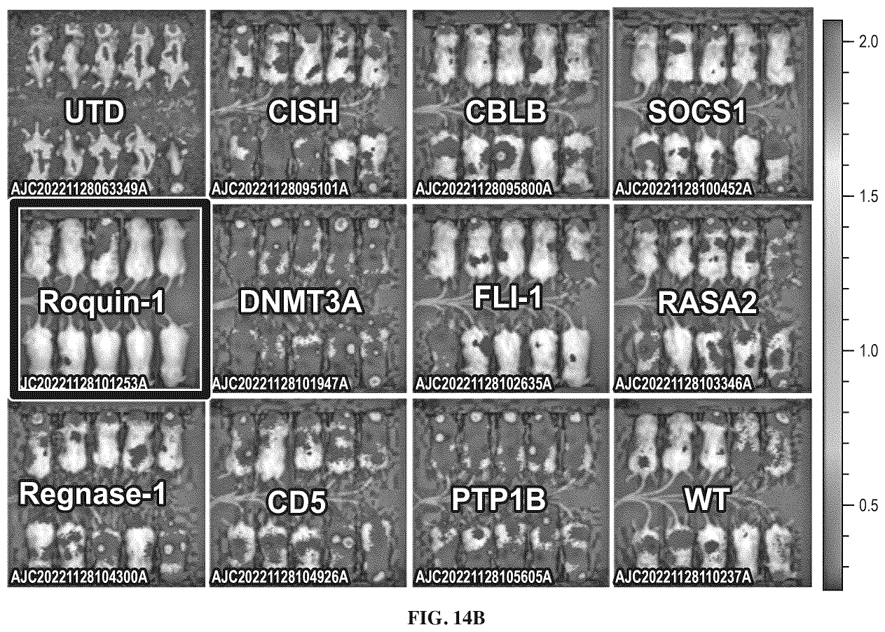

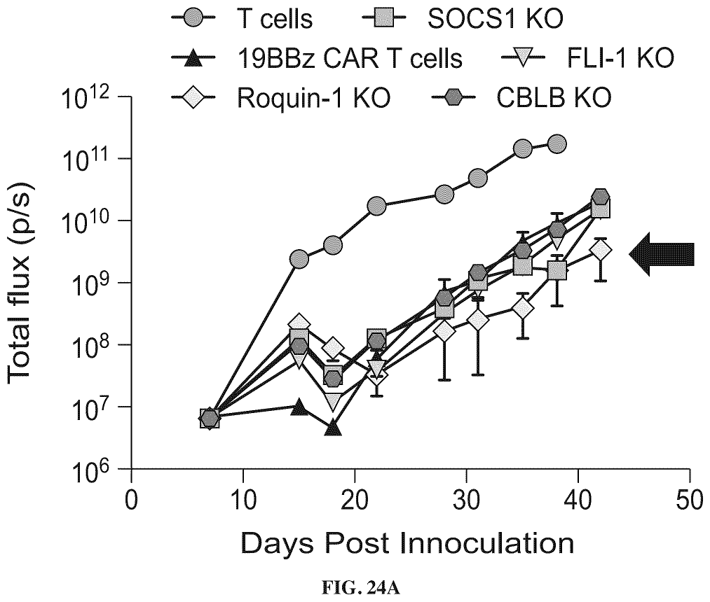

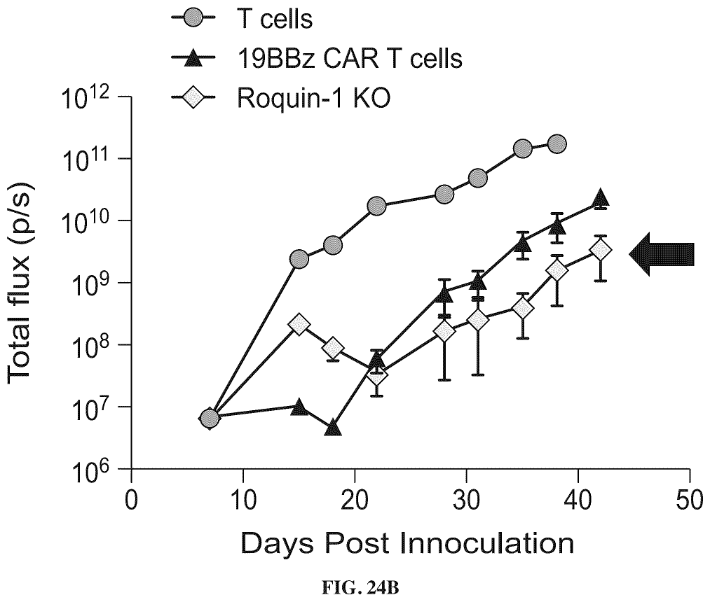

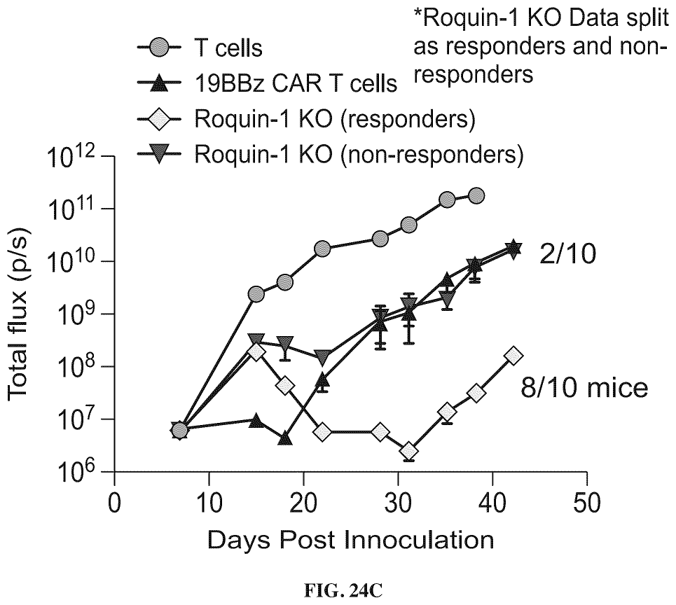

In a second experiment, anti-tumor activity of CAR-T cells base edited using the base editor ABE8.20m in combination with a guide polynucleotide corresponding to EF01, EF02, EF07, EF08, EF15, EF20, EF96, EF25, EF94, or EF4 (see Tables 1, 2A, and 2B for sequences) to reduce or eliminate expression of CISH, CBLB, SOCS1, Roquin-1, DNMT3A, FLI-1, RASA2, Regnase-1, CD5, or PDP1B, respectively, was evaluated using a sub-therapeutic mantle cell lymphoma (MCL) model involving the intravenous administration of 5E5 JeKo-1 cells (a mantle cell lymphoma (MCL) cell line isolated from the peripheral blood mononuclear cells of a 78-year-old female with a large cell variant of MCL showing leukemic conversion) expressing luciferase into 6-7 week-old NSG female mice (Jackson Labs: Stock 05557) at day zero (0) and subsequently infusing 2.5E5 CAR-T cells (N=10 for each base-edited CAR-T cell and N=10 or 5 for each non-base-edited cells) into the mice at day 7. Groups were randomized based on mouse body weight. Tumor clearance was monitored over time (2×/wk) by measuring luciferase flux in photons per second (p/s) in the mice using an IVIS® Spectrum in vivo imaging system, and tumor size was also evaluated at the termination of the experiment using bioluminescence imaging. Also, measurements of mouse bodyweights were measured and clinical observations were collected 2×/wk. Some of the base edited CAR-T cells (e.g., CAR-T cells base edited to reduce or eliminate expression of Roquin-1) showed prolonged tumor control in the MCL model. Some of the base edited CAR-T cells (e.g., CAR-T cells base edited to knock-out expression of CBLB, SOCS1, FLI-1, or Roquin-1) showed improved clearance of the JeKo-1 cells from the mice relative to unedited CAR-T cells (FIGS. 14A, 14B, 24A, 24B, and 24C). Often (10) mice administered Roquin-1 knock-out CAR-T cells, eight (8) were observed to be responders showing higher reductions in JeKo-1 cells than mice administered CAR-T cells expressing Roquin-1 (FIG. 24C).

Therefore, base editing of CAR-T cells to reduce or eliminate expression of polypeptide selected from those listed in Table 8 (alterations that may be referred to as “efficacy edits”) was an effective strategy for reducing susceptibility of the CAR-T cells to developing an exhausted phenotype after continuous or multiple antigen exposures and/or improving the efficacy of the CAR-T cells in killing target cells in vitro and/or in vivo. Table 9 provides a summary of data gathered in the above-described experiments relating to the CAR-T cells.

TABLE 9
Summary of data for CAR-T cells edited to knock-out expression
of polypeptides selected from those listed in Table 8.

| Name of | | | | Cytotoxicity | | |
|---|---|---|---|---|---|---|
| guide RNA | | | | after repeat | Repeated | In vivo |
| used to | | On- | | stim. | stim. | activity |
| knock-out | | target | | (AUC² | Expansion | (AUC¹ |
| the target | Target | editing | Protein KO | WT/AUC | (fold exp. | WT/AUC |
| gene | Gene | efficiency | (WB)¹ | edit) | over WT) | edit) |
| EF1 | CISH | 88% | Yes | 1.6 | 1.5 | — |
| EF7 | SOCS1 | 93% | Yes | 1.8 | 2 | — |
| EF8 | Roquin-1 | 97% | Yes by ELISA | 2.8 | 4 | 38 |
| EF15 | DNMT3A | 95% | Yes | 1.4 | 1 | — |
| EF20 | FLI1 | 97% | Yes | 0.9 | 1 | 11 |
| EF51 | DGKa | 82% | Yes | 1.1 | 2 | — |
| EF54 | DGKz | 88% | Reduced | 1.2 | 2.5 | 20 |
| | | | expression | | | |
| EF62 | PRKACA | 82% | Reduced | 1.3 | 1 | 0.5 |
| | | | expression | | | |
| EF65 | PTPN6 | 88% | Reduced | 1.8 | 1 | — |
| | | | expression | | | |
| EF70 | EIF2A | 85% | Yes | 1.8 | 2.5 | 0.6 |
| EF96 | RASA2 | 93% | Yes | 1.8 | 2 | 0.7 |
| EF101 | DHX37 | 94% | Yes | 2.2 | 0.5 | — |

¹Protein knockout was measured using Western blots (WB) unless indicated otherwise.
²Area under curve.

### Example 2: Roquin-1 Knock-Out Chimeric Antigen Receptor (CAR) T Cells Showed Improved Function Relative to Unedited Cells after being Exposed to the Target Antigen

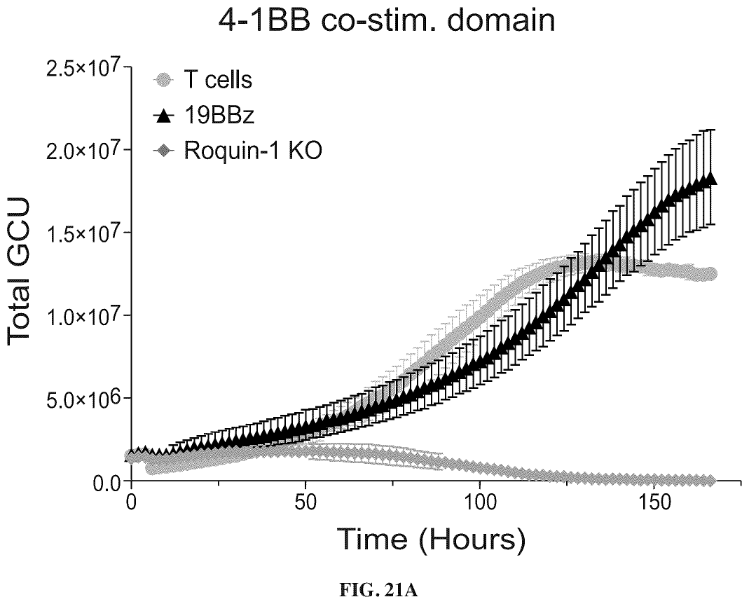

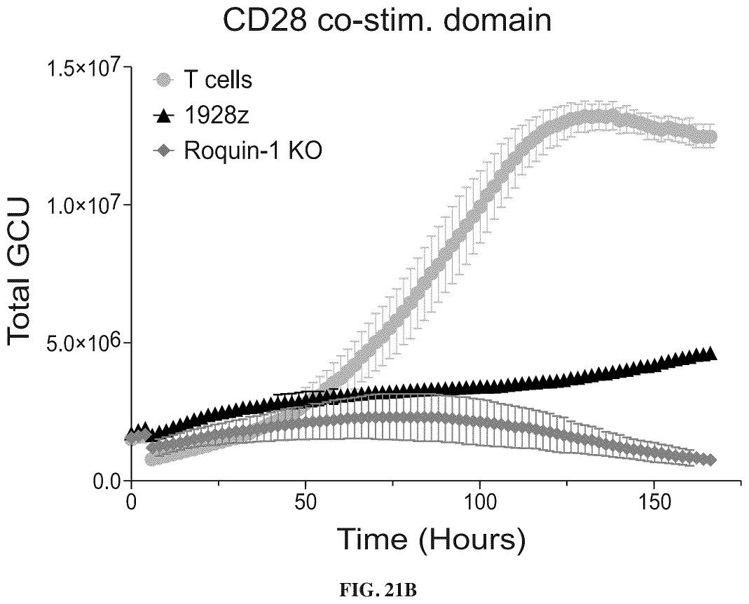

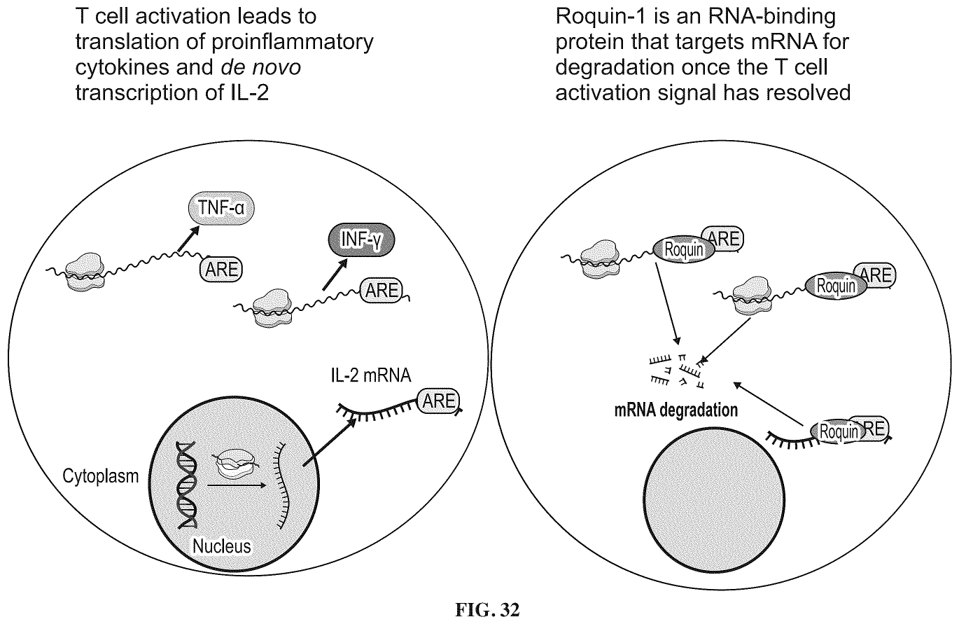

Experiments were undertaken to demonstrate that (KO) chimeric antigen receptor (CAR) T cells modified as described in Example 1 to knock-out expression of Roquin-1 showed improved function both in vitro and in vivo relative to CAR-T cells expressing Roquin-1 after being exposed either continuously and/or multiple times in succession to the antigen targeted by the CAR-T cells. Not intending to be bound by theory, knock-out of Roquin-1 may improve T cell function because Roquin-1 is a polypeptide involved in mRNA degradation (FIG. 32). Binding of Roquin-1 (e.g., at the 3′ untranslated region (UTR) of mRNA) targets an mRNA for degradation. Roquin-1 mRNA targets include, as non-limiting examples, 1) mRNAs associated with DNA replication and cell proliferation (e.g., IRF4, Pola1, Prim1, and Prim2), 2) pro-inflammatory cytokines (e.g., TNFa, IL-2), and 3) T cell costimulatory receptors (e.g., ICOS, OX40). Improved cytotoxicity of anti-CD19 CAR T cells modified to knock-out expression of Roquin-1 was observed across cells expressing CAR polypeptides having different co-stimulatory domains. Roquin-1 KO T cells expressing a chimeric antigen receptor (CAR) polypeptide containing an anti-CD19 scFv as an antigen-binding domain, either a 4-1BB costimulatory domain or a CD28 co-stimulatory domain, and a CD3ζ signaling domain showed increased cytotoxicity relative to CAR-T cells expressing Roquin-1 when co-cultured with JeKo-1 cells at an effector-to-target (E:T) ratio of 1:5 (FIGS. 21A and 21B) following six consecutive plate-bound antigen exposures carried out as described in FIG. 3A.

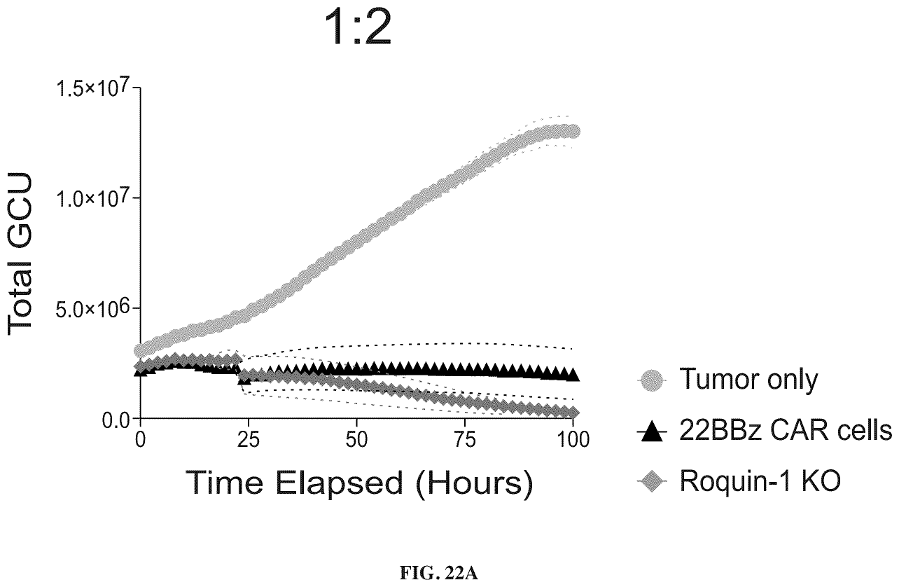

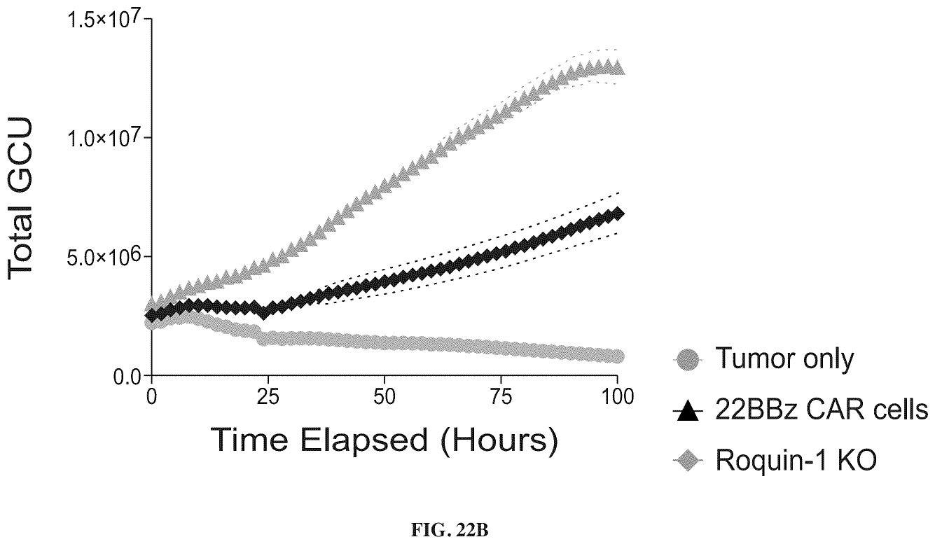

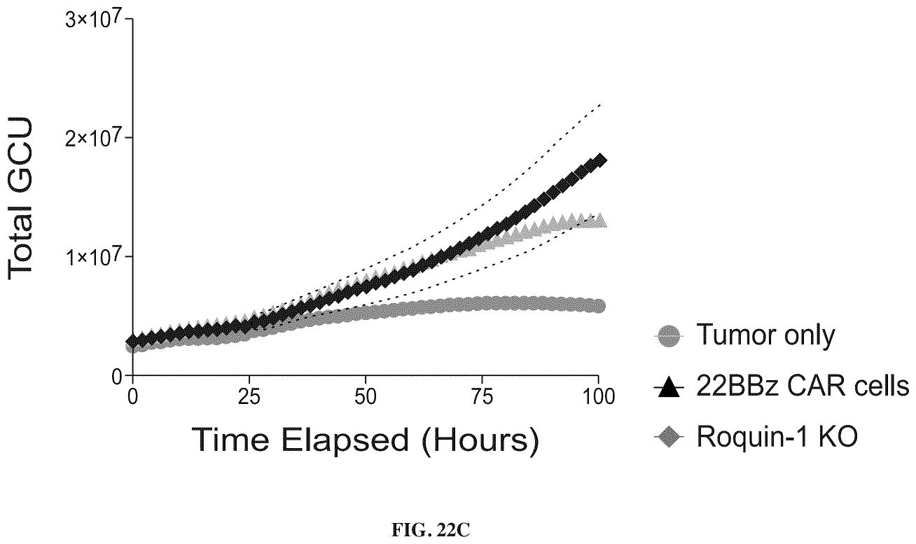

The improved cytotoxicity of CAR-T cells modified to knock-out expression of Roquin-1 was observed across CAR-T cells expressing different antigen binding domains. For example, the increase in cytotoxicity described above for CAR-T cells expressing a chimeric antigen receptor (CAR) polypeptide containing an anti-CD19 scFv as an antigen-binding domain, a 4-1BB costimulatory domain, and a CD3ζ signaling domain was also observed in CAR-T cells expressing a CAR polypeptide containing an anti-CD22 scFv as an antigen-binding domain, a 4-1BB costimulatory domain, and a CD3ζ signaling domain (FIGS. 22A to 22C). The CAR-T cells were co-cultured with Nalm-6 cells at effector-to-target ratios (E:T) of 1:2, 1:5, and 1:10 following four consecutive plate-bound antigen stimulations carried out as described in FIG. 3A. The increase in cytotoxicity was observed across all E:T ratios evaluated.

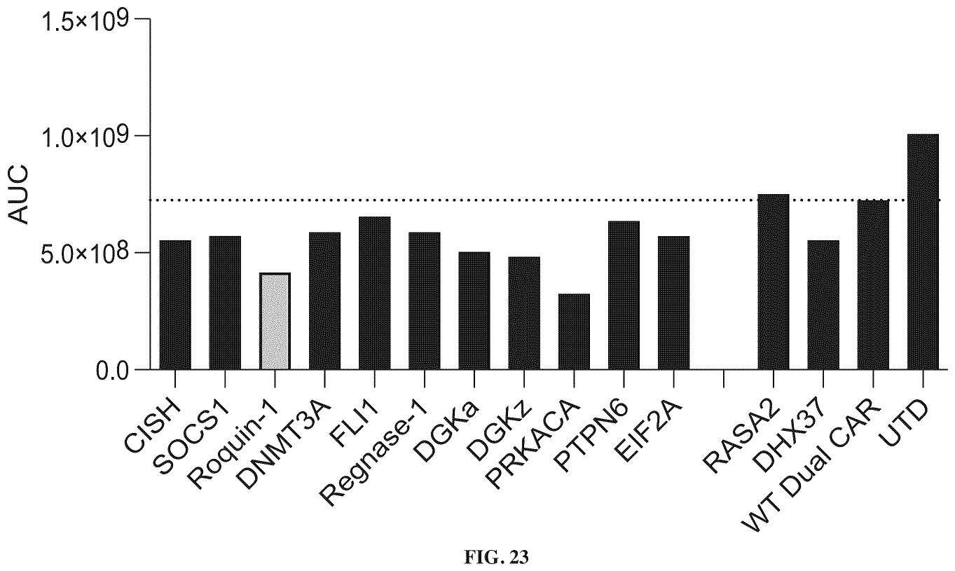

Increased cytotoxicity of CAR-T cells modified as described in Example 1 to knock-out expression of Roquin-1, as well as other polypeptides listed in Table 8, relative to unmodified CAR-T cells was observed for CAR-T cells expressing two different chimeric antigen receptors (CARs) (FIG. 23). One of the two CAR polypeptide contained an anti-CD19 scFv domain, a 4-1BB costimulatory domain, and a CD3ζ signaling domain, and the other CAR polypeptide contained an anti-ROR1 scFv domain, a CD28 costimulatory domain, and a CD3ζ signaling domain. The CAR-T cells were co-cultured with JeKo-1 cells expressing green fluorescent protein (GFP) at an effector-to-target ratio (E:T) of 1:5.

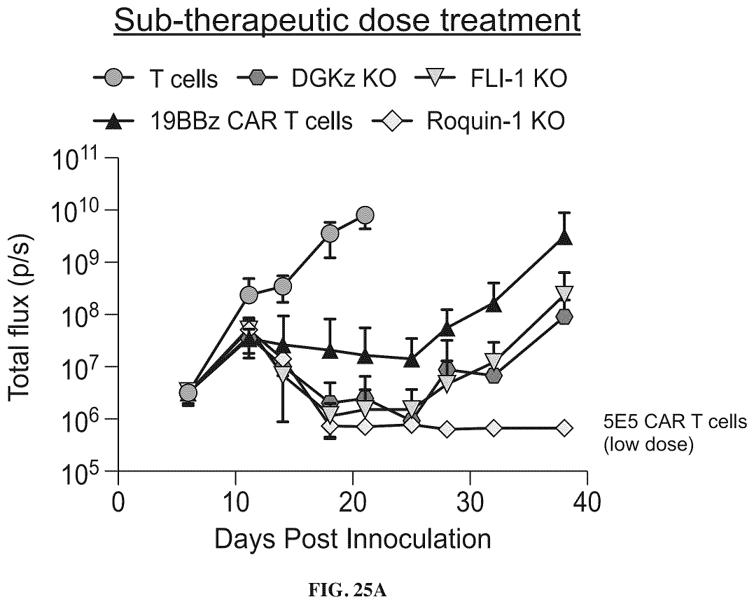

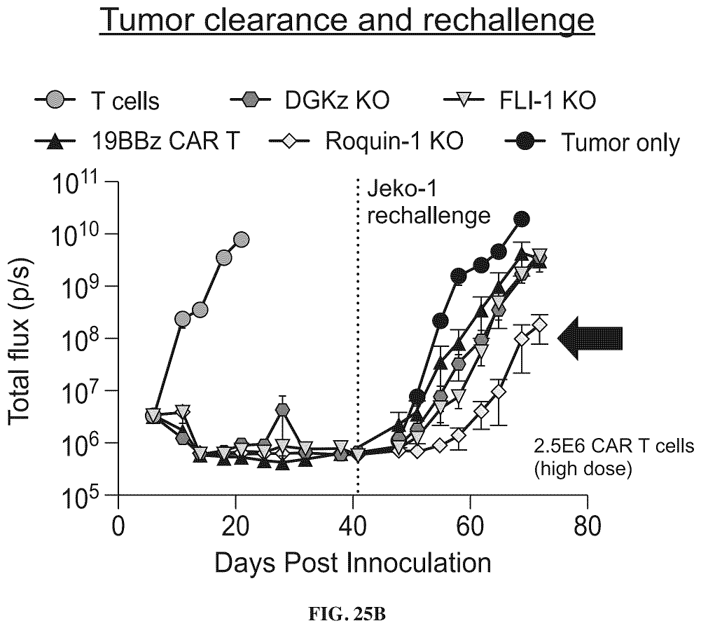

An experiment was undertaken to demonstrate, in vivo, that CAR-T cells modified as described in Example 1 to knock out expression of Roquin-1 showed improved ability to clear tumors in a mouse and control tumor clearance relative to unmodified CAR-T cells and CAR-T cells modified to knock-out expression of DGKz or FLI-1. The CAR-T cells expressed a CAR polypeptide containing an anti-CD19 scFv as an antigen-binding domain, a 4-1BB costimulatory domain, and a CD3ζ signaling domain. Anti-tumor activity of the CAR-T cells was evaluated using a sub-therapeutic (FIG. 25A) or tumor clearance and rechallenge (FIG. 25B) mantle cell lymphoma (MCL) model involving the infusion (“inoculation”) of 5E5 JeKo-1 expressing luciferase into mice at day zero (0) and subsequently infusing 2.5E5 CAR-T cells (FIG. 25A; “low dose”) or 2.5E6 CAR-T cells (FIG. 25B; “high dose”) into the mice at day 7. Mice administered the Roquin-1 KO CAR-T cells at the low dose showed increased reductions in levels of the JeKo-1 cells relative to CAR-T cells expressing Roquin-1 (FIG. 25A). Mice previously administered the Roquin-1 KO CAR-T cells at the high dose at day 7 and then re-inoculated with JeKo-1 cells at about day 40 (i.e., about 33 days following administration of the CAR-T cells) also showed increased reductions in levels of the JeKo-1 cells relative to CAR-T cells expressing Roquin-1 (FIG. 25B).

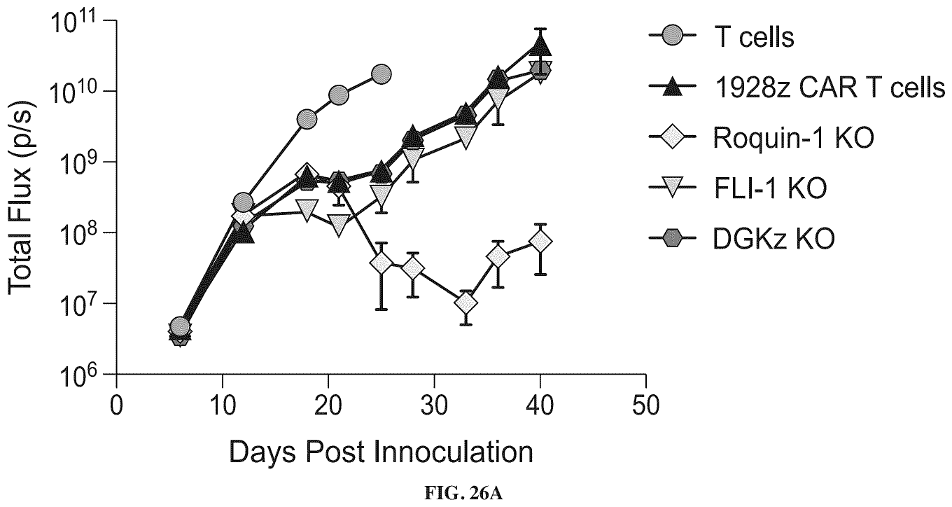

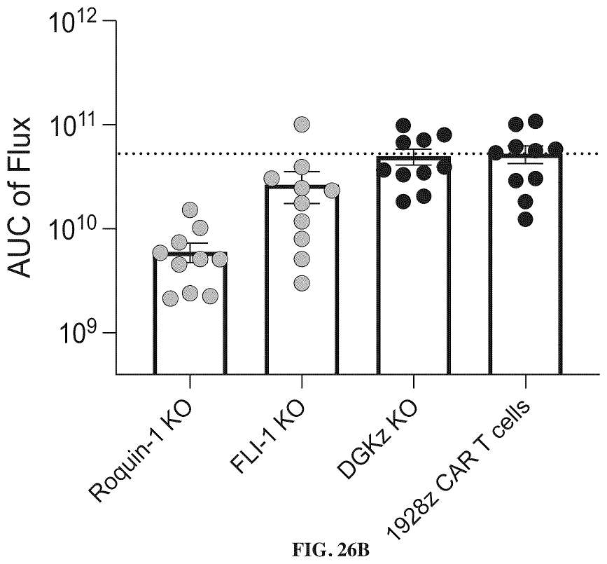

The improved ability of the Roquin-1 knock-out (KO) CAR-T cells to clear tumors in the mice relative to unmodified CAR-T cells or CAR-T cells modified to knock-out expression of DGKz or FLI-1 was found to be independent of the co-stimulatory domain of the chimeric antigen receptors expressed by the cells. For example, Roquin-1 KO CAR-T cells expressing a CAR polypeptide containing an anti-CD19 scFv as an antigen-binding domain, a CD28 costimulatory domain rather than the 4-1BB costimulatory domain used in the above in vivo experiment, and a CD3ζ signaling domain also showed improved ability to clear tumors in mice (FIGS. 26A and 26B). Anti-tumor activity of the CAR-T cells was evaluated using a sub-therapeutic mantle cell lymphoma (MCL) model involving the infusion (“inoculation”) of 5E5 JeKo-1 cells expressing luciferase into mice at day zero (0) and subsequently infusing 2.5E5 CAR-T cells (“low dose”) into the mice at day 7. Mice administered the Roquin-1 KO CAR-T cells showed increased reductions in levels of the JeKo-1 cells relative to CAR-T cells expressing Roquin-1 (FIGS. 26A and 26B).

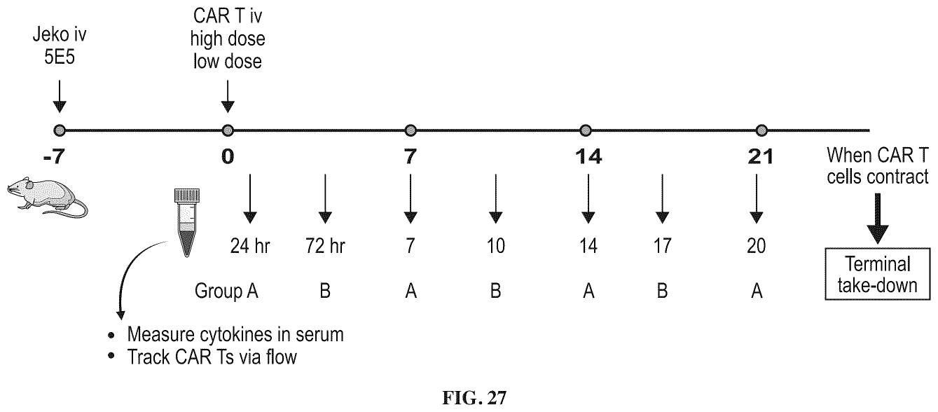

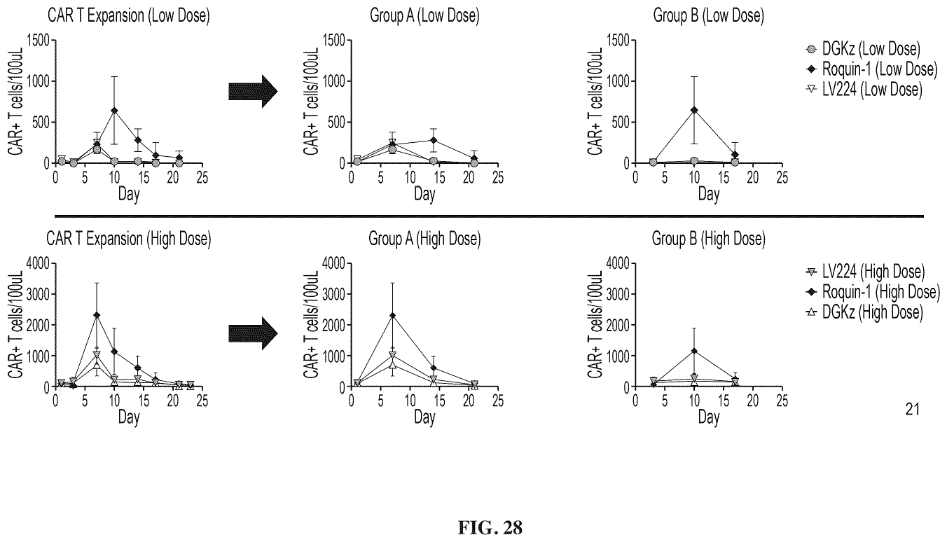

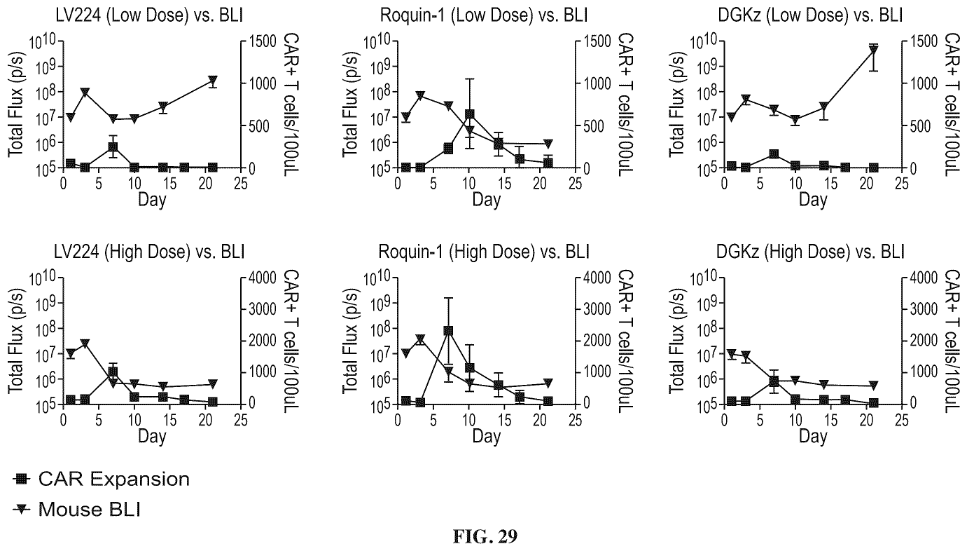

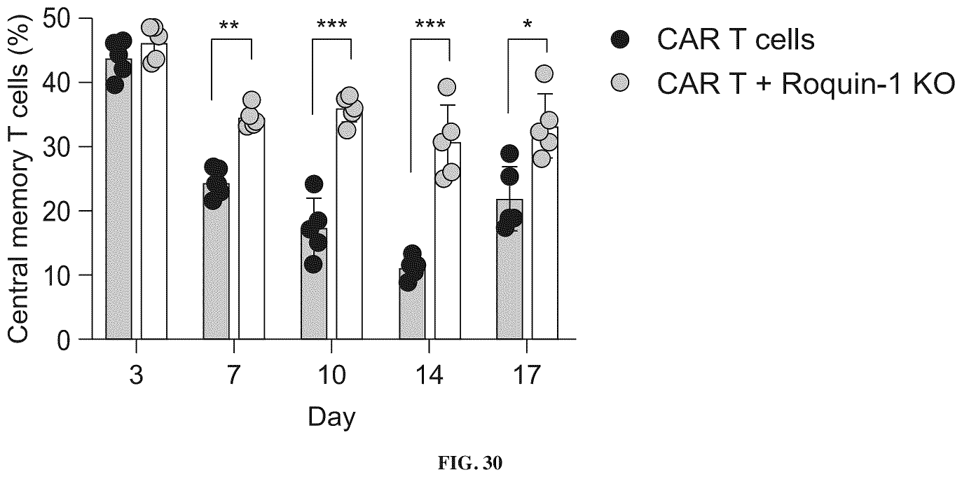

An experiment as described in FIG. 27 and Table 10 was undertaken to demonstrate improved expansion kinetics (i.e., increased rates of proliferation) and increased cytokine secretion levels for T cells expressing a chimeric antigen receptor (CAR) polypeptide modified as described in Example 1 to knock-out expression of Roquin-1 or DGKz relative to cells expressing Roquin-1 and DGKz. The chimeric antigen receptor polypeptides contained an anti-CD19 scFv as an antigen-binding domain, a 4-1BB costimulatory domain, and a CD3ζ signaling domain. Peak CAR T cell expansion was detected between 7 and 10 days following administration of the CAR T cells to the mice (FIG. 28). CAR T cell expansion kinetics correlated with detected tumor burden in vivo (FIG. 29). The Roquin-1 KO CAR-T cells also maintained the central memory phenotype over time (FIG. 30) more than CAR-T cells expressing Roquin-1.

TABLE 10
Description of groups of mice evaluated.

| | | | | | | Guide | |
|---|---|---|---|---|---|---|---|
| | | | | | | RNAs Used | |
| | | | | | | to Edit the | |
| | | | | | | Cells to | |
| | | | | | | Knock-Out | |
| | | | | | Target | Expression | |
| | Tumor | Tumor Cells | Number | | Gene | of the | CAR+ Cells |
| | Cell | Administered | of Mice | | Knocked | Target | Administered |
| Group | Line | Per Mouse | (N) | CAR³ | Out | Gene | Per Mouse |
| 1A | JeKo-1 | 5E+5 | 5 | 19BBz | — | — | 5E+5 |
| 1B | JeKo-1 | 5E+5 | 5 | 19BBz | — | — | 5E+5 |
| 2A | JeKo-1 | 5E+5 | 5 | 19BBz | Roquin-1 | EF08 | 5E+5 |
| 2B | JeKo-1 | 5E+5 | 5 | 19BBz | Roquin-1 | EF08 | 5E+5 |
| 3A | JeKo-1 | 5E+5 | 5 | 19BBz | DGKz | EF54 | 5E+5 |
| 3B | JeKo-1 | 5E+5 | 5 | 19BBz | DGKz | EF54 | 5E+5 |
| 4A | JeKo-1 | 5E+5 | 5 | 19BBz | — | — | 2.5E+6 |
| 4B | JeKo-1 | 5E+5 | 5 | 19BBz | — | — | 2.5E+6 |
| 5A | JeKo-1 | 5E+5 | 5 | 19BBz | Roquin-1 | EF08 | 2.5E+6 |
| 5B | JeKo-1 | 5E+5 | 5 | 19BBz | Roquin-1 | EF08 | 2.5E+6 |
| 6A | JeKo-1 | 5E+5 | 5 | 19BBz | DGKz | EF54 | 2.5E+6 |
| 6B | JeKo-1 | 5E+5 | 5 | 19BBz | DGKz | EF54 | 2.5E+6 |
| 7 | JeKo-1 | 5E+5 | 5 | UTD | n/a | n/a | 2.5E+6 |

³The term “19BBz” indicates a chimeric antigen receptor (CAR) polypeptide containing an anti-CD19 scFv as an antigen-binding domain, a 4-1BB costimulatory domain, and a CD3ζ signaling domain. The term “UTD” indicates untransduced cells that do not express any CAR polypeptide.

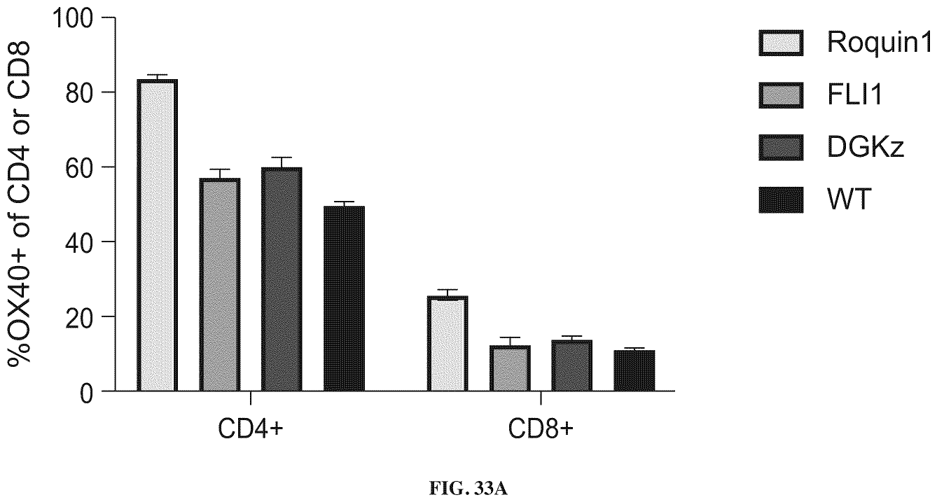

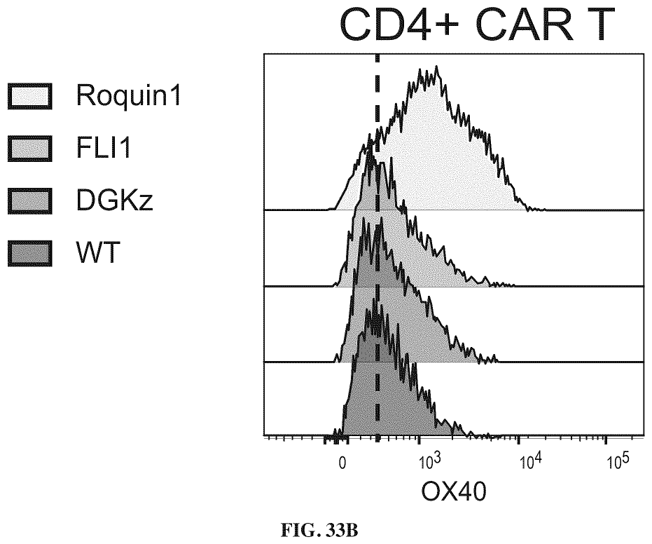

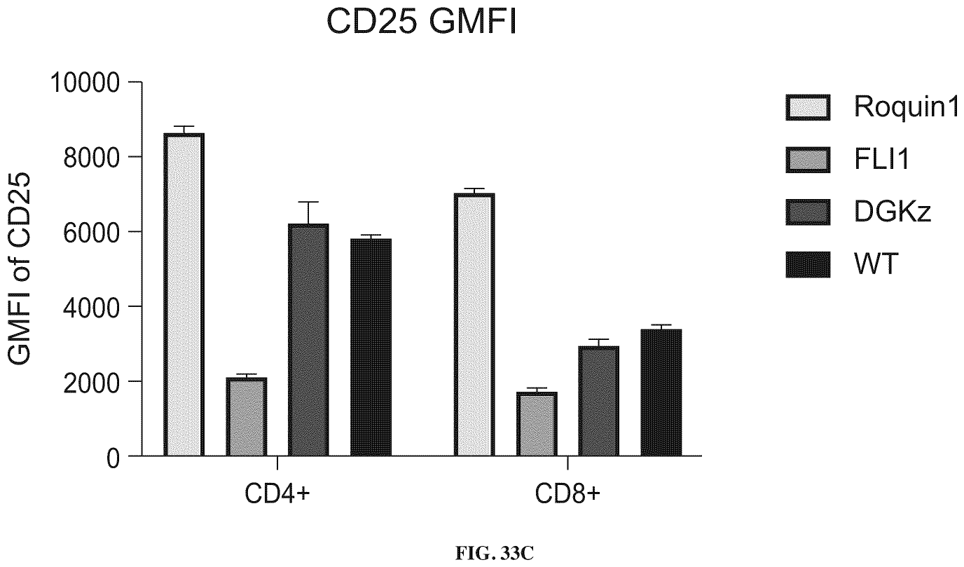

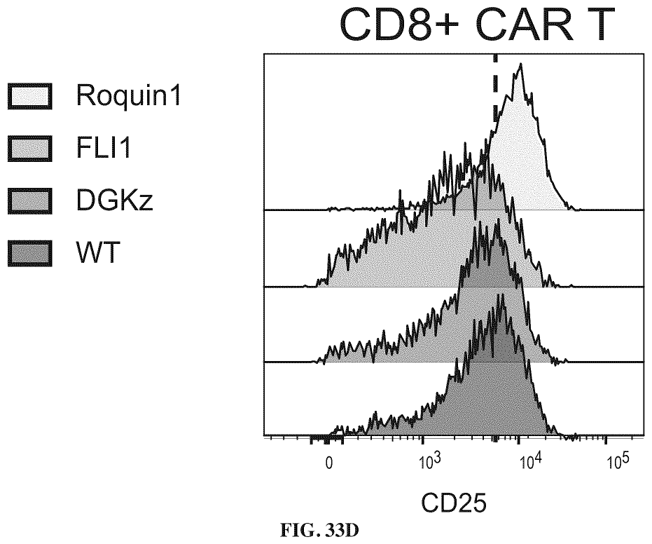

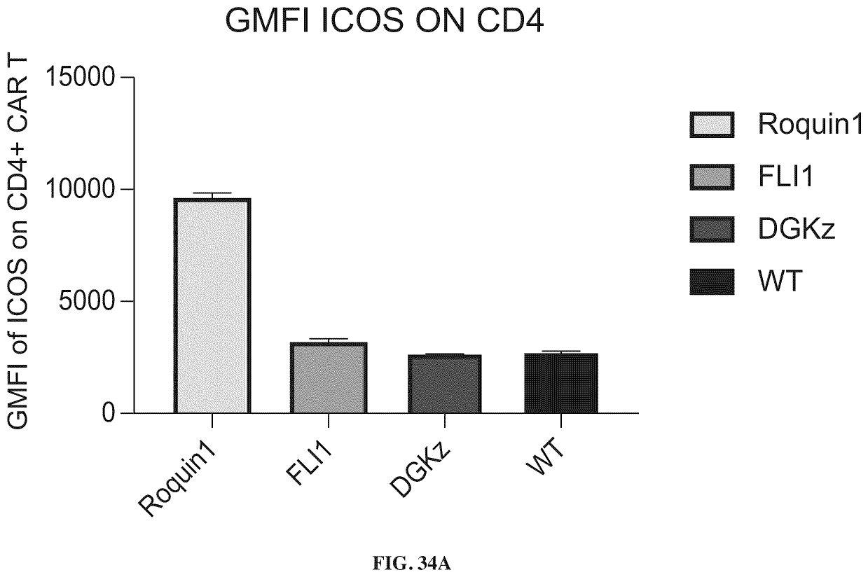

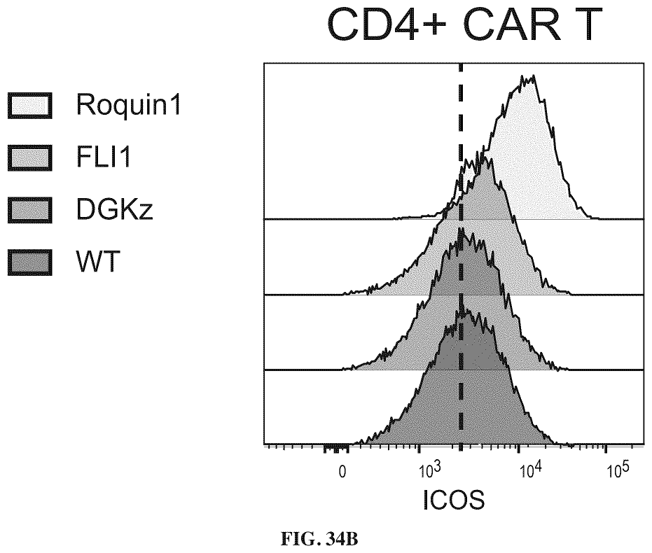

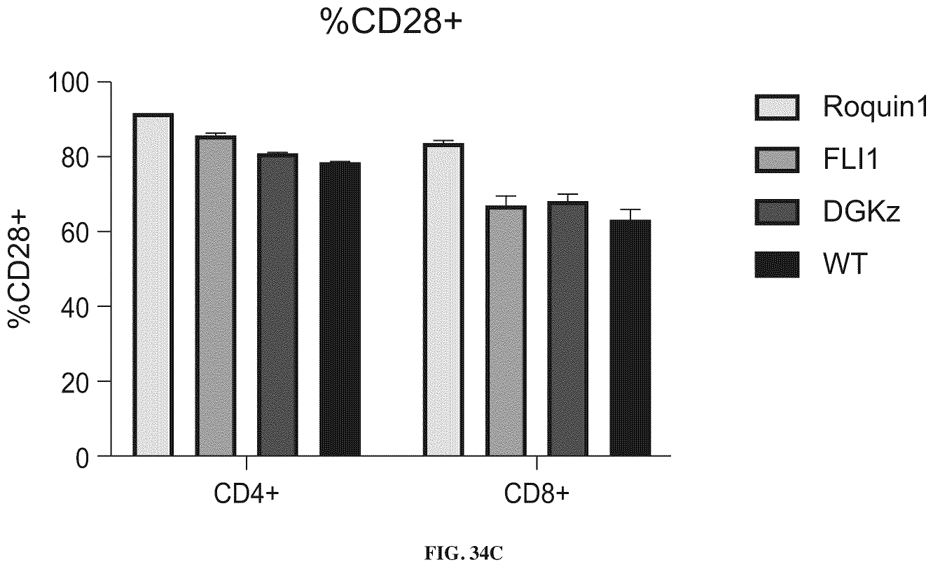

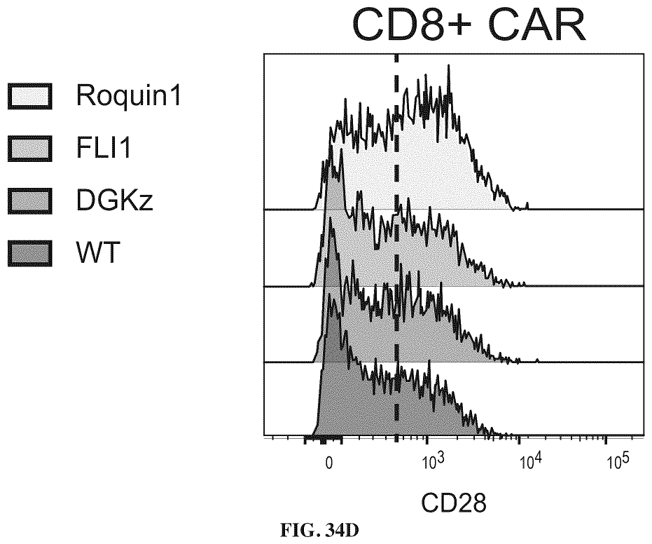

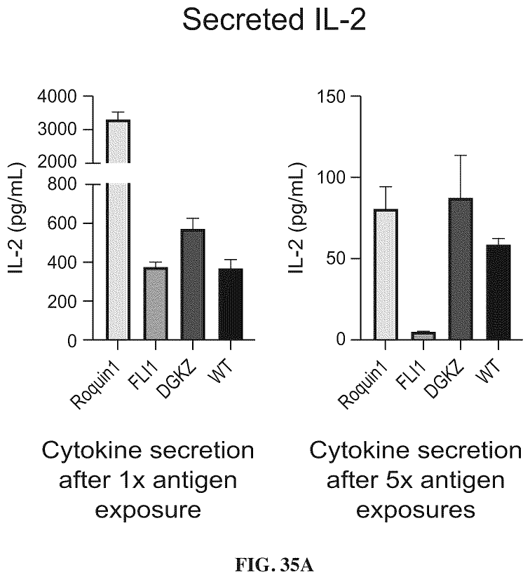

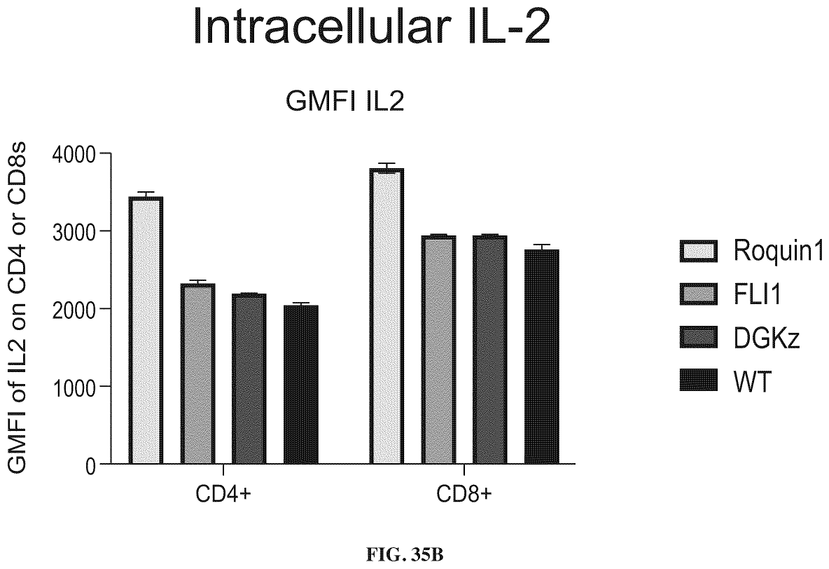

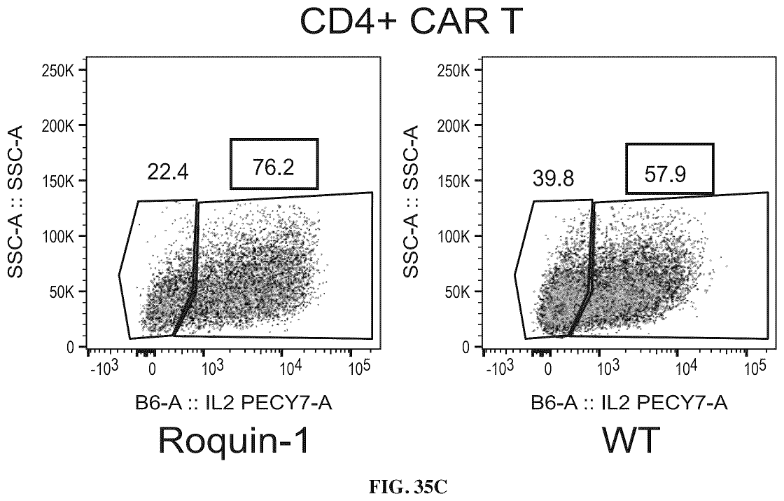

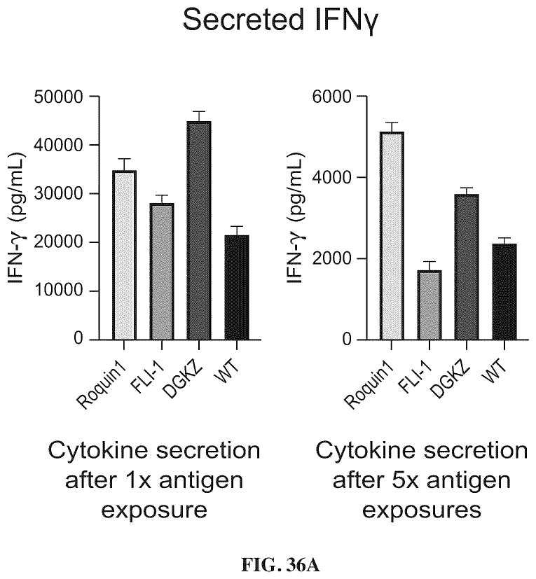

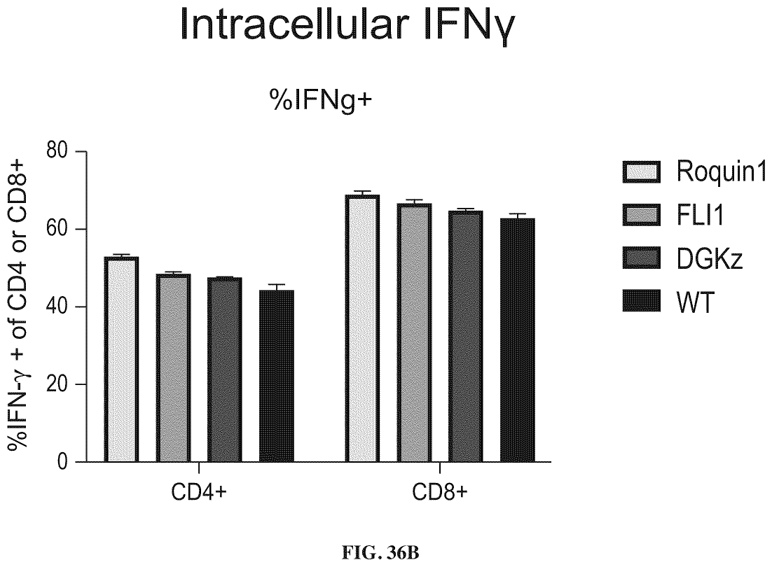

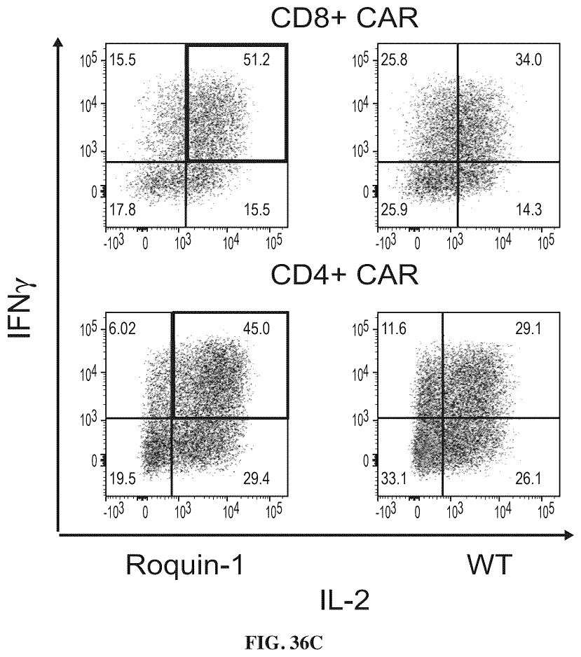

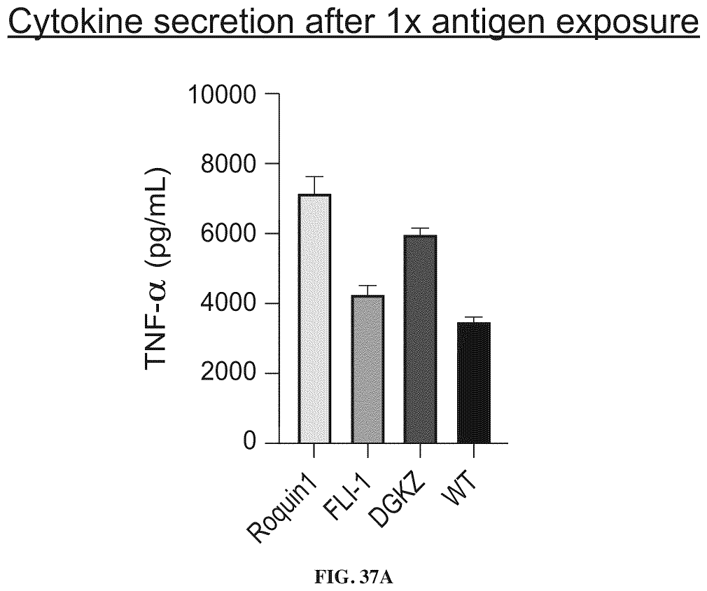

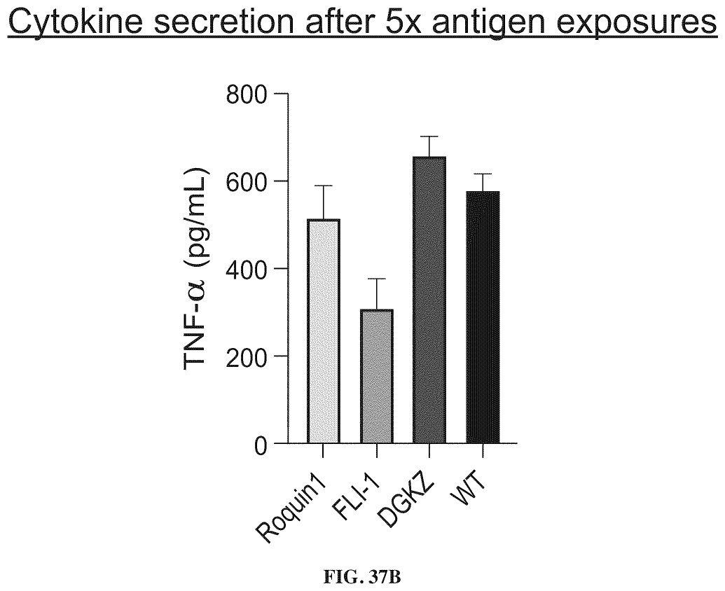

Experiments were undertaken to demonstrate that knock-out of Roquin-1 expression in CAR-T cells led to an increase in expression of activation markers (e.g., OX40, CD25), co-stimulatory signals (e.g., ICOS, CD28), and secretion of cytokines (e.g., IL-2, IFNγ, and TNFα) relative to CAR-T cells expressing Roquin-1 or expressing Roquin-1 and modified as described in Example 1 to knock-out expression of DGKz or FLI-1. The CAR-T cells expressed a chimeric antigen receptor containing an anti-CD19 scFv as an antigen-binding domain, a 4-1BB costimulatory domain, and a CD3ζ signaling domain. When the Roquin-1 CAR-T cells were co-cultured for 24-hours with JeKo-1 cells following being stimulated through exposure to antigen 4 times, the Roquin-1 CAR-T cells showed increased expression of the activation markers OX40 (FIGS. 33A and 33B) and CD25 (FIGS. 33C and 33D) and of the T cell co-stimulatory signals ICOS (FIGS. 34A and 34B) and CD28 (FIGS. 34C and 34D) relative to the CAR-T cells expressing Roquin-1 or expressing Roquin-1 and modified to knock-out expression of DGKz or FLI-1. OX40 signaling promotes T cell division and cytokine production, and CD25 expression is associated with T cell growth and proliferation. Inducible T-cell costimulatory (ICOS) is an inducible co-stimulator expressed on activated CD4+ T cells, and cluster of differentiation 28 (CD28) acts as a major co-stimulatory receptor in promoting activation of naïve T cells. When the Roquin-1 CAR-T cells were co-cultured for 24-hours with Raji cells following being stimulated through exposure to antigen either 1 or 5 times, the Roquin-1 CAR-T cells showed increased expression of the cytokines IL-2 (FIGS. 35A to 35C), interferon gamma (IFNγ) (FIGS. 36A to 36C), and TNF-alpha (FIGS. 37A and 37B) relative to the CAR-T cells expressing Roquin-1.

The above-described experiments establish knock-out of expression of Roquin-1 as an effective strategy for reducing susceptibility of CAR-T cells to developing an exhausted phenotype after continuous or multiple antigen exposures and/or improving the efficacy of the CAR-T cells in killing target cells in vitro and/or in vivo.

### Example 3: Disruption of Roquin-1 Expression in Immune Cells Using Prime Editing

A prime editor construct together with a paired prime editing guide RNA (pegRNA), with or without a secondary nicking guide RNA (nRNA), may be used to induce targeted, programmable changes to genomic DNA. These targeted changes may involve frameshift insertion/deletion (indel) mutations resulting in a premature stop codon that disrupts endogenous expression of Roquin-1 in allogeneic human immune cells (e.g. T cells). Alternatively, or additionally, these targeted changes may involve transversion and/or transition mutations that may disrupt endogenous expression of Roquin-1 in allogeneic human immune cells (e.g., T cells) through various mechanisms, such as splice site disruption, start site disruption, active site disruption, promoter or enhancer disruption, protein structure disruption, or stop codon insertion. Additional mutations may be encoded into the pegRNA to increase the frequency or product purity of a mutation introduced to a polynucleotide using prime editing. Methods for editing polynucleotide sequences using prime editing are well known in the art (see, e.g., Petrova I O, Smirnikhina S A. The Development, Optimization and Future of Prime Editing. Int J Mol Sci. 2023 Dec. 1; 24(23):17045. doi: 10.3390/ijms242317045, the disclosure of which is incorporated herein in its entirety by reference for all purposes).

The pegRNA and/or nRNA sequences each contain a spacer selected from one or more of the following: CCUAAAGUUAAUUAUGUACC (SEQ ID NO: 876), GGAUGCCAGUUCCUUGGACC (SEQ ID NO: 877), GCUAGGGGAUGCCAGUUCCU (SEQ ID NO: 878), UGGGCAGCAGUAAGGGCUAG (SEQ ID NO: 879), and UUGGGCAGCAGUAAGGGCUA (SEQ ID NO: 880). The pegRNAs contain the following scaffold sequence:

(SEQ ID NO: 881)
GUUUUAGAGCUAGAAAUAGCAAGUUAAAAUAAGGCUAGUCCGUUAUCAA
CUUGAAAAAGUGGCACCGAGUCGGUGC.

Experiments are undertaken to disrupt expression of Roquin-1 in T cells. Primary human T cells are first cultured at 10⁶ cells/mL in complete medium comprising ImmunoCult™ XF T Cell Expansion Medium (Stem-Cell Technologies), 1% Penicilin-Streptomycin, 2 mM GlutaMax™ and 25 mM HEPES Buffer (Life Technologies), and 5% CTS™ ImmuneCell SR (ThermoFisher). The complete medium also contains 5 ng/mL human IL-15 (Biolegend) and 10 ng/mL IL-7 (Biolegend). T cells are stimulated with ImmunoCult Human CD3/CD28/CD2 T Cell Activator (Stem-Cell Technologies) following the manufacturer's instructions and then incubated at 37° C., 5% CO² and 95% humidity. Two-days post-activation, T cells are counted, washed with sterile PBS (Gibco) and resuspended in P3 buffer (Lonza) at 10⁷ cells/mL. T cells are electroporated with 1 mg of Roquin-1-specific pegRNA (see Table 14), 0.5 mg of Roquin-1-specific nRNA (see Table 15) and 2 mg of mRNA encoding the prime editor construct per 10⁶ cells using the Lonza 4D Nucleofector system (program DH-102). T cells are allowed to recover in complete medium at 10⁶ cells/mL and medium is exchanged every other day to adjust T cell concentration to 5×10⁵ cells mL⁻¹ until day 10 when cells are cryopreserved in CS10 for later analysis.

The following methods were employed in the above examples.

### Repeated Antigen Stimulation

Cells were exposed to antigen multiple times according to the method shown in FIG. 3A. Specifically, a recombinant human CD19 (rhCD19) antigen-coated plate used to stimulate the CAR-T cells was prepared at day −1 either by contacting anti-his-tag coated plate wells overnight at 4° C. with a 5 ng/μL solution of human CD19 his-tag recombinant protein available from Thermo Fisher (50 μL per well of a 96-well plate, 180 μL per well of a 48-well plate, 300 μL per well of a 24-well plate, 550 μL per well of a 12-well plate, or 1.5 mL per well of a 6-well plate), or by contacting anti-Fc coated plate wells overnight at 4° C. with a 5 ng/μL solution of recombinant human CD19-Fc chimera (carrier-free) available from BioLegend (50 μL per well of a 96-well plate, 180 μL per well of a 48-well plate, 300 μL per well of a 24-well plate, 550 μL per well of a 12-well plate, or 1.5 mL per well of a 6-well plate). CAR-T cells in XF Base Medium and pre-counted using either a NucleoCounter® NC-200™ automated cell analyzer or using a NucleoCounter® NC-250™ (8-channel) where the cells were stained using an acridine orange/propidium iodide viability stain, were added to the rhCD19 antigen-coated plate. The following cell culture volumes and total cell counts were added to wells for each of the following plate types: 96-well plate wells were loaded with about 5e4 total cells in 200 μL media; 48-well plate wells were loaded with about 1.8e-5 cells in about 400 μL media; 24-well plate wells were loaded with about 3e5 cells in about 1 mL media; 12-well plate wells were loaded with about 6e5 cells in about 2 mL media; and 6-well plate wells were loaded with about 2e6 cells in 4 mL media. The cells were then cultured in the rhCD19 antigen-coated plate wells until day 3, at which time the cells were either transferred to a newly-prepared recombinant human CD19 antigen-coated plate well prepared as described above starting on the previous day (i.e., day 2), or evaluated in an experiment as described above. Cells were transferred to a newly-prepared antigen-coated plate every three days, as appropriate, until the cells were exposed to the CD19 antigen the number of times desired.

### XF Base Medium

XF Base Medium was prepared by combining 500 mL ImmunoCult™ XF media from STEMCELL™ Technologies, 25 mL CTS™ supplement, 5 mL GlutaMAX™ supplement, and 5 mL of a HEPES buffer. The XF Base Medium was sterilized using filtration.

### Base Editing of Human T Cells

To base edit human T cells, 1 mL of frozen primary human T cells in a cryo tube were thawed on day 1 in a 37° C. water bath and transferred to a 15-mL conical tube containing 9 mL cell media (XF Base Medium). The cells were then spun at 500×g for 5 minutes and the cell media was removed from the resulting cell pellet. The cell pellet was resuspended in 10 mL media and the number of cells in the media was then determined. The cells were placed in a T75 flask and incubated overnight. Then, on day 2, the number of cells in the media was again determined and the concentration of the cells in the media was adjusted to be approximately 1e6 cells/mL. The cells were then activated using 26 25 μL of a commercially available ImmunoCult™ CD3/CD28/CD2 T cell activator media supplement for activation of T cells per 1 mL of cell media. Interleukin-7 (IL-7) and interleukin-15 (IL-15) were both then added to the cell media. The cells were incubated in the cell media and the cells were counted on day 5. On day 5 the cells were electroporated. The cells were electroplated using cuvettes or wells containing a maximum total cell count of about 5e+6 (cuvette) or 1e+6 (well) cells, 2 μg/μL each of mRNA (5 μL total for cuvette and 1 μL total for well) encoding a base editor and a guide RNA molecule (2.5 μL total for cuvette and 0.5 μL total for well), and P3 electroporation buffer. The cells electroporated using a commercially available electroporation device and fresh media was then added to the electroporated cells along with IL-7 and IL-15. Subsequently, IL-7 and IL-15 was added to the cell media every 2-3 days.

### Cell Lysis and Protein Quantitation

Cells were lysed for Western Blotting (WB) using a lysis solution containing a commercially available Radio-Immunoprecipitation Assay (RIPA) Lysis and Extraction Buffer and a phosphatase inhibitor cocktail. To lyse the cells, about 1-5e+6 cells washed once using phosphate buffer solution were resuspended in about 100-500 μL of the lysis solution and incubated on ice for about 30 minutes. In some cases, lysis was accelerated by performing about three freeze-thaw cycles of the cells suspended in the lysis solution using dry ice. The lysed cells were then centrifuged at 14,000×g for about 10 minutes. The resulting supernatant was transferred to a clean tube and stored at −20° C. for later use.

Protein concentrations were measured using a commercially available Pierce™ 660 nm protein assay, which is a rapid ready-to-use colorimetric method for measuring protein concentration.

### Measurement of Cytokine Secretion Levels

Cytokine secretion levels (i.e., levels of GZMB, IFNg, IL-2, and/or TNFa) were measured using the commercially available Ella™ system from BioAgilytix Labs, LLC for conducting microfluidics-based immunoassays. To measure cytokine secretion levels, at day −1, about 1e5 antigen-positive tumor cells were co-cultured in a wells with about 1e5 CAR+ T cells in triplicate. The total volume of each co-culture was about 200 μL. The cells were cultured in XF Base Medium. The co-cultures were incubated for about 24 hours. At day 0, the wells were spun at 500g for 5 minutes and the Ella™ system was used according to the manufacturer's protocols to quantify cytokine levels in the resulting supernatant.

### Flow Cytometry Stain Protocol

Flow cytometry was used to characterize base editing efficiency (e.g., reduced or eliminated target protein expression), cell identity (e.g., percentage of cells remaining in culture within various lymphocyte and monocyte compartments), chimeric antigen receptor (CAR) expression (e.g., percentage of T cells expressing a CAR), memory phenotype, and/or activation state (e.g., percentage of cells upregulating CD25 and CD69) in T cells. First, cells were counted using a NucleoCounter NC-200™ automated cell counter. Then, between about 2e5 and 1e6 cells to be evaluated were transferred to a well of a 96-well plate. The plate was then spun down at 500×g for about 5 minutes and the supernatant was removed. The cells were then washed in about 200 μL of flow cytometry staining buffer (FACS Buffer containing phosphate buffered saline and 2% fetal bovine serum). The cells were then stained using a near-infra red (NIR) live/dead stain followed by staining using one or more (e.g., panels) fluorochrome-labeled antibodies described in Table 11. The antibody-stained cells were then fixed using paraformaldehyde and subsequently evaluated using a MACSQuant™ Analyzer 16 flow cytometer. In some cases, compensation was carried out using CultraComp eBeads™ by combining 1 μL of antibody with 1 drop of UltraComp eBeads following by fixing using paraformaldehyde.

TABLE 11
Description of antibodies and fluorochromes linked thereto
used to immunostain cells for flow cytometry.

| Antibody Target | |
|---|---|
| Polypeptide | Fluorochrome |
| B2M | PE |
| CCR7 | APC |
| CD14 | VioBlue |
| CD19 | PE |
| CD2 | PEVio770 |
| CD25 | APC |
| CD27 | BV605 |
| CD4 | VioBright 515 |
| CD4 | BV510 |
| CD45 | VioGreen |
| CD45RA | VioBlue |
| CD5 | APC |
| CD5 CAR | FITC |
| CD52 | PE |
| CD56 | VioBright 667 |
| CD62L | PE |
| CD69 | VioBlue |
| CD8 | PerCPVio700 |
| CD8 | BV650 |
| EGFR | PE-Cy7 |
| GM-CSF | BV421 |
| GzmB | PE-CF594 |
| HLADR | BV421 |
| IFNg | PE |
| IL-2 | APC |
| PD1 (CD279) | VioBright 515 |
| Perforin | PerCP-Cy5.5 |
| TCRab | PerCPVio700 |
| TNFa | BV605 |

The following representative flow cytometry gating strategies were used:

### To Characterize Cell Identity

1. All Cells→Single Cells→CD45+→Live/Dead (L/D)→CD56−→CD2+→Report CD2+% of CD45+CD56− cells
2. All Cells→Single Cells→CD45+→L/D→CD56−→CD2+→CD4/CD8→Report CD4/CD8 split of CD2+ cells
3. All Cells→Single Cells→CD45+→L/D→CD56+→Report CD56+% of Live CD45+ cells
4. All Cells→Single Cells→CD45+→L/D→CD14+→Report CD14+% of Live CD45+ cells
5. All Cells→Single Cells→CD45+→L/D→CD19+→Report CD19+% of Live CD45+ cells

### To Characterize Editing Efficiency

1. All Cells→Single Cells→CD45+→L/D→CD2+→TCRab+→Report TCRab+% of CD2+ cells
2. All Cells→Single Cells→CD45+→L/D→CD2+→CD7+→Report CD7+% of CD2+ cells
3. All Cells→Single Cells→CD45+→L/D→CD2+→CD52+→Report CD52+% of CD2+ cells
4. All Cells→Single Cells→CD45+→L/D→CD2+→PD1+→Report PD1+% of CD2+ cells

### To Characterize CAR Expression

1. All Cells→Single Cells→CD45+→L/D→CD7 FITC+→Report CAR+% of Live CD45+ cells
2. All Cells→Single Cells→CD45+→L/D→CD8/CD7 FITC→Report CAR+% of CD8+ cells

### To Characterize T Cell Activation

1. All Cells→Single Cells→CD45+→L/D→CD2+→CD25+→Report CD25+% of CD2+ cells
2. All Cells→Single Cells→CD45+→L/D→CD2+→CD69+→Report CD69+% of CD2+ cells

### Preparation of Base Edited CAR-T Cells

Base edited chimeric antigen receptor (CAR) T cells were typically prepared as follows. On day −1 or day 0 T cells were thawed and subsequently activated using ImmunoCult™ CD3/CD28/CD2 T cell activator media supplement. On day 2, the T cells were counted and adjusted to a concentration of 1e6/mL. Then, 1 mL of the cell culture were added to a well of a 6-well plate and add to the cell culture poloxamer synperonic F108 and lentiviral particles containing a polynucleotide encoding a chimeric antigen receptor. The cells were subsequently incubated overnight and fresh XF Base Medium was then added to the well. On day 5 or day 6, the cells were base edited by electroporating the cells with mRNA encoding a base editor and a guide RNA, as described above. Cytokines were added to the cell media every 2-3 days. At day 12 or day 13, between about 50 k and 100 k cells were added to a 96-well U-bottom plate for pelleted and stored at −20° C. for later analysis using next-generation sequencing (NGS). The cells were also analyzed as described above using flow cytometry. Cells were cryopreserved in 100% CryoStor® CS10 freeze media or in a composition containing 50% PlasmaLyte and 40% 100% CryoStor® CS10 freeze media.

### CAR-T Cell Cytotoxicity Assays

Cytotoxicity of T cells was evaluated using an IncuCyte® live-cell analysis system for real-time quantitative live-cell imaging and analysis that enabled visualization and quantitation of cell behavior over time by automatically gathering and analyzing images within a laboratory incubator. At day −1, 50 μL of poly-L-ornithine (0.01%) was added to each well of a 96-well plate and incubated at room temperature for 30 minutes. Then, the poly-L-omithine (a coating used to enhance cell attachment and adhesion to surfaces) solution was removed from the plate and the plate was allowed to dry at room temperature for 30 minutes. Then, 100 μL of cell culture media (XF Base Medium) containing antigen-positive tumor cells (e.g., about 20,000 cells) was added to wells of the 96-well plate. The plate was then placed into the IncuCyte® live-cell analysis system and the system was programmed to take four images of green fluorescent protein (GFP) expression in each well every three hours. At day 0, about 100 μL of cell culture media containing CAR-T cells (e.g., about 4e3 cells) was added to the wells containing the antigen-positive tumor cells. Each condition was evaluated in triplicate. The 96-well plate was then added back to the IncuCyte® live-cell analysis system programmed to take four images of green fluorescent protein (GFP) expression in each well every three hours.

Cytotoxicity of CAR-T cells was also measured by measuring luciferase activity in target antigen-positive tumor cells. Co-cultures were prepared in 96-well plates. Each co-culture had a total volume of about 200 μL and contained a total of about 50 k or 100 k target cells. The co-cultures were incubated and luminescence was measured at 24 hr, 48 hr, and 72 hr by adding D-luciferin to the co-cultures to be evaluated at each time point, incubating the resulting mixtures for 10 minutes at 37° C. and subsequently measuring luminescence using a spectrophotometer commercially available from Tecan.

### Intracellular Cytokine Staining

Intracellular cytokine staining was used to measure cytokine/chemokine expression in CAR-T cells (effector cells) that were stimulated in vitro by being co-cultured in wells for about 6-hours with antigen-positive tumor cells (target cells) according to the following protocol. First, co-cultures were prepared containing about 1.5e5 cells/well of the effector cells and about 1.5e5 cells/well of the target cells. The final concentration of cells in the co-culture was about 1.5e6 cells/mL in complete medium containing no exogenous cytokines. The total volume of each co-culture was about 200 μL. As a positive control, some effector cells were cultured in the absence of the target cells and stimulated using a cell stimulation cocktail containing paramethoxyamphetamine (PMA) and ionomycin. To each cell culture was added an anti-CD107 antibody (CD107a) labeled using a BV650 fluorophore/dye. The cell cultures were then incubated for about 1 hour at 37° C. under 5% CO₂. Then, the protein transport inhibitors brefeldin A (Golgi Plug) and monesin (Golgi Stop) were added to each cell culture and the cells were incubated for between about 4 and 5 hours. The cells were stained using viability eFluor780 stain and labeled anti-CD4, CD8, and EGFR antibodies. The cells were then permeabilized using a FIX & PERM™ Cell Permeabilization Kit available from ThermoFisher Scientific and evaluated using flow cytometry, as described above, after immunostaining the cells to detect intracellular expression of the cytokines granulocyte-macrophage colony-stimulating factor (GM-CSF), interferon-gamma (INF-γ), tumor necrosis factor (TNF), interleukin-2 (IL-2), and granzyme B (GzmB).

### Western Blots

For evaluation of protein expression using Western blots, lysed cell supernatant samples were first prepared as described above. The supernatant samples were then denatured by adding dithiothreitol (DTT) to each sample and incubating for 5 min at 95° C. The denatured samples were then combined with primary antibodies specific for a target antigen of interest and for a control target antigen (e.g., β-actin/GAPDH). The samples were then combined with a secondary antibody (e.g., anti-rabbit antibody linked to horseradish peroxidase (HRP) and/or anti-mouse antibody linked to a near-infra red (NIR) dye). The immunostained samples were then evaluated using chemiluminescence and/or fluorescence detection using a Jess™ instrument for size-based automated capillary Western blot assays.

### Amino Acid and Nucleotide Sequences for the Anti-CD19 Chimeric Antigen Receptors

An amino acid sequence of the anti-CD19 chimeric antigen receptor (CAR) used in the Examples is provided below. An amino acid sequence of the CAR fused by way of a self-cleaving polypeptide to an epidermal growth factor receptor (EGFR) tag and alternatively referred to as “LV224” is also provided below. In the below amino acid sequences, T-cell surface glycoprotein CD8 alpha chain signal peptides are indicated by BOLD-UNDERLINED UPPERCASE TEXT, an immunoglobulin light chain variable region is indicated by DOUBLE-UNDERLINE ALL CAPS TEXT, linkers are indicated by bold text, an immunoglobulin heavy chain variable region is indicated by DOUBLE-UNDERLINE BOLD ALL CAPS TEXT, a CD8a hinge domain is indicated by double-underlined text, a CD8a transmembrane domain is indicated by bold italic text, a 4-1BB costimulatory domain is indicated by underlined text, a CD3z domain is indicated by bold-dash-underlined text, a T2A self-cleaving peptide is indicated by bold lowercase text, an epidermal growth factor receptor tag is indicated by lowercase plain text.
Anti-CD19 CAR amino acid sequence:

(SEQ ID NO: 830)
MALPVTALLLPLALLLHAARPDIQMTQTTSSLSASLGDRVTISCRASQDISKYLNWYQQKPD
GTVKLLIYHTSRLHSGVPSRFSGSGSGTDYSLTISNLEQEDIATYDCQQGNTLPYTFGGGTK
LEITGGGGSGGGGSGGGGSEVKLQESGPGLVAPSQSLSVTCTBSGVSLPDYGVSWIRQPPPK
GLEWLGVIWGSETTYYNSALKSRLTIIKDNSKSQVFLKMNSLQTDDTAIYYCAKHYYYGGSY
AMDYWGQGTSVTVSSTTTPAPRPPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEGGC

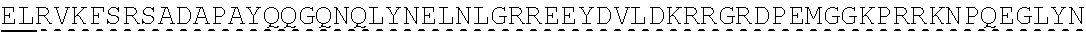

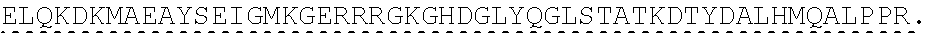

LV224 amino acid sequence:

(SEQ ID NO: 831)
MALPVTALLLPLALLLHAARPDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTBSGVSLPDYGVSWIRQPPPK
GLEWLGVIWGSETTYYNSALKSRLTIIKDNSKSQVFLKMNSLQTDDTAIYYCAKHYYYGGSY
AMDYWGQGTSVTVSSTTTPAPRPPTPAPTIASQPLSLRPEACRPAAGGAVHTRGLDFACDIY
IWAPLAGTCGVLLLSLVITLYCKRGRKKLLYIFKQPFMRPVQTTQEEDGCSCRFPEEEEGGC

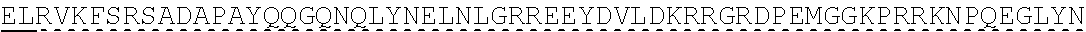

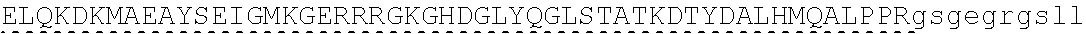

tcgdveenpgpMALPVTALLLPLALLLHAARPGSrkvcngigigefkdslsinatnikhfkn
ctsisgdlhilpvafrgdsfthtppldpqeldilktvkeitgflliqawpenrtdlhafenl
eiirgrtkqhgqfslavvslnitslglrslkeisdgdviisgnknlcyantinwkklfgtsg
qktkiisnrgensckatgqvchalcspegcwgpeprdcvscrnvsrgrecvdkcnllegepr
efvenseciqchpeclpqamnitctgrgpdnciqcahyidgphcvktcpagvmgenntlvwk
yadaghvchlchpnctygctgpglegcptngpkipsiatgmvgalllllvvalgiglfm.

Representative nucleotide sequence encoding the anti-CD19 CAR:

(SEQ ID NO: 832)
ATGGCCCTGCCCGTTACCGCCCTACTCCTGCCCCTGGCCCTCCTGCTCCACGCCGCCAGGCC
CGACATCCAGATGACCCAGACCACCAGCAGCCTGTCCGCCAGCCTGGGGGACAGGGTGACCA
TCTCCTGTCGGGCCTCTCAGGACATCAGCAAGTACCTGAATTGGTACCAGCAGAAGCCTGAC
GGCACCGTGAAGCTGCTGATCTACCACACCAGCCGGCTGCACTCCGGCGTGCCCAGCCGGTT
CAGCGGCAGCGGGTCAGGCACCGACTACAGCCTGACCATCTCCAACCTGGAGCAGGAGGACA
TCGCCACCTACTTCTGCCAGCAGGGGAACACCCTGCCATACACCTTTGGCGGGGGCACCAAG
CTGGAGATCACCGGGGGGGGCGGGAGCGGCGGCGGCGGGTCCGGGGGGGGCGGCTCCGAGGT
GAAGCTGCAGGAGTCCGGCCCCGGACTGGTCGCCCCCTCCCAGAGCCTGTCCGTGACATGCA
CCGTGTCCGGCGTGAGCCTGCCCGACTACGGCGTGTCATGGATCAGGCAGCCCCCCCGGAAG
GGGCTGGAGTGGCTGGGGGTGATCTGGGGGAGCGAGACCACTTACTACAACAGTGCTCTCAA
GAGCAGGCTGACCATCATCAAGGACAACAGCAAGAGCCAGGTGTTCCTGAAGATGAACAGCC
TGCAGACCGATGACACCGCCATCTACTACTGTGCCAAGCACTACTACTATGGCGGCAGCTAC
GCCATGGACTACTGGGGGCAGGGGACAAGCGTGACTGTGAGCAGCACCACCACCCCTGCCCC
CAGGCCCCCCACCCCCGCCCCCACCATCGCCAGCCAGCCCCTGTCCCTGCGGCCCGAGGCCT
GCCGGCCCGCCGCCGGCGGCGCCGTGCACACCCGGGGCCTGGACTTCGCCTGCGACATCTAC
ATCTGGGCCCCCCTGGCCGGAACCTGCGGCGTGCTGCTGCTGTCCCTGGTGATCACCCTGTA
CTGCAAGCGGGGCCGGAAGAAGCTGCTGTACATCTTCAAGCAGCCCTTCATGCGCCCCGTGC
AGACCACCCAGGAGGAGGACGGCTGCAGCTGCAGGTTCCCCGAGGAGGAGGAGGGGGGCTGC
GAGCTGCGGGTGAAGTTCAGCAGGAGCGCCGACGCCCCTGCTTACCAGCAGGGCCAGAATCA
GCTGTACAACGAGCTGAATCTGGGCCGGAGGGAGGAGTACGACGTGCTGGACAAGCGGAGGG
GCAGGGACCCCGAGATGGGCGGCAAGCCCAGGCGGAAGAACCCCCAGGAGGGGCTGTACAAC
GAGCTGCAGAAGGACAAGATGGCCGAGGCCTACTCCGAGATCGGCATGAAGGGCGAGAGGCG
GAGAGGCAAGGGCCATGACGGCCTGTACCAGGGCCTGTCCACCGCCACCAAGGACACCTACG
ACGCCCTGCACATGCAGGCCCTGCCCCCCCGC.

Representative nucleotide sequence encoding LV224:

(SEQ ID NO: 833)
ATGGCCCTGCCCGTTACCGCCCTACTCCTGCCCCTGGCCCTCCTGCTCCACGCCGCCAGGCC
CGACATCCAGATGACCCAGACCACCAGCAGCCTGTCCGCCAGCCTGGGGGACAGGGTGACCA
TCTCCTGTCGGGCCTCTCAGGACATCAGCAAGTACCTGAATTGGTACCAGCAGAAGCCTGAC
GGCACCGTGAAGCTGCTGATCTACCACACCAGCCGGCTGCACTCCGGCGTGCCCAGCCGGTT
CAGCGGCAGCGGGTCAGGCACCGACTACAGCCTGACCATCTCCAACCTGGAGCAGGAGGACA
TCGCCACCTACTTCTGCCAGCAGGGGAACACCCTGCCATACACCTTTGGCGGGGGCACCAAG
CTGGAGATCACCGGGGGGGGGGGGAGCGGCGGCGGCGGGTCCGGGGGGGGCGGCTCCGAGGT
GAAGCTGCAGGAGTCCGGCCCCGGACTGGTCGCCCCCTCCCAGAGCCTGTCCGTGACATGCA
CCGTGTCCGGCGTGAGCCTGCCCGACTACGGCGTGTCATGGATCAGGCAGCCCCCCCGGAAG
GGGCTGGAGTGGCTGGGGGTGATCTGGGGGAGCGAGACCACTTACTACAACAGTGCTCTCAA
GAGCAGGCTGACCATCATCAAGGACAACAGCAAGAGCCAGGTGTTCCTGAAGATGAACAGCC
TGCAGACCGATGACACCGCCATCTACTACTGTGCCAAGCACTACTACTATGGCGGCAGCTAC
GCCATGGACTACTGGGGGCAGGGGACAAGCGTGACTGTGAGCAGCACCACCACCCCTGCCCC
CAGGCCCCCCACCCCCGCCCCCACCATCGCCAGCCAGCCCCTGTCCCTGCGGCCCGAGGCCT
GCCGGCCCGCCGCCGGCGGCGCCGTGCACACCCGGGGCCTGGACTTCGCCTGCGACATCTAC
ATCTGGGCCCCCCTGGCCGGAACCTGCGGCGTGCTGCTGCTGTCCCTGGTGATCACCCTGTA
CTGCAAGCGGGGCCGGAAGAAGCTGCTGTACATCTTCAAGCAGCCCTTCATGCGCCCCGTGC
AGACCACCCAGGAGGAGGACGGCTGCAGCTGCAGGTTCCCCGAGGAGGAGGAGGGGGGCTGC
GAGCTGCGGGTGAAGTTCAGCAGGAGCGCCGACGCCCCTGCTTACCAGCAGGGCCAGAATCA
GCTGTACAACGAGCTGAATCTGGGCCGGAGGGAGGAGTACGACGTGCTGGACAAGCGGAGGG
GCAGGGACCCCGAGATGGGCGGCAAGCCCAGGCGGAAGAACCCCCAGGAGGGGCTGTACAAC
GAGCTGCAGAAGGACAAGATGGCCGAGGCCTACTCCGAGATCGGCATGAAGGGCGAGAGGCG
GAGAGGCAAGGGCCATGACGGCCTGTACCAGGGCCTGTCCACCGCCACCAAGGACACCTACG
ACGCCCTGCACATGCAGGCCCTGCCCCCCCGCGGCAGCGGAGAGGGCAGAGGAAGTCTTCTA
ACATGCGGTGACGTGGAGGAGAATCCCGGCCCTATGGCCCTGCCTGTGACAGCTCTGCTGCT
TCCTCTGGCACTGCTGCTGCATGCTGCCAGACCAGGCAGCAGAAAAGTGTGCAACGGCATCG
GCATCGGAGAGTTCAAGGACAGCCTGAGCATCAACGCCACCAACATCAAGCACTTCAAGAAC
TGCACCAGCATCAGCGGCGACCTGCACATTCTGCCTGTGGCCTTTAGAGGCGACAGCTTCAC
CCACACACCTCCACTCGATCCCCAGGAGCTGGACATCCTGAAAACCGTGAAAGAGATCACCG
GCTTTCTGCTGATCCAGGCTTGGCCCGAGAACCGGACAGATCTGCACGCCTTCGAGAACCTG
GAAATCATCAGAGGCCGGACCAAGCAGCACGGCCAGTTTTCTCTGGCTGTGGTGTCCCTGAA
CATCACCAGCCTGGGCCTGAGAAGCCTGAAAGAAATCAGCGACGGCGACGTGATCATCTCCG
GCAACAAGAACCTGTGCTACGCCAACACCATCAACTGGAAGAAGCTGTTCGGCACCAGCGGC
CAGAAAACAAAGATCATCAGCAACCGGGGCGAGAACAGCTGCAAGGCTACAGGCCAAGTGTG
CCACGCTCTGTGTAGCCCTGAAGGCTGTTGGGGACCCGAGCCTAGAGATTGCGTGTCCTGCA
GAAACGTGTCCCGGGGCAGAGAATGCGTGGACAAGTGCAATCTGCTGGAAGGCGAGCCCCGC
GAGTTCGTGGAAAACAGCGAGTGCATCCAGTGTCACCCCGAGTGTCTGCCCCAGGCCATGAA
CATTACCTGTACCGGCAGAGGCCCCGACAACTGTATTCAGTGCGCCCACTACATCGACGGCC
CTCACTGCGTGAAAACATGTCCTGCTGGCGTGATGGGAGAGAACAACACCCTCGTGTGGAAG
TATGCCGACGCCGGACATGTGTGCCACCTGTGTCACCCTAATTGCACCTACGGCTGTACAGG
CCCTGGCCTGGAAGGCTGTCCAACAAACGGACCTAAGATCCCCTCTATCGCCACCGGCATGG
TTGGAGCCTTGCTGCTTCTGCTGGTGGTGGCCCTTGGCATCGGCCTGTTTATG.

Further amino acid and nucleotide sequences for chimeric antigen receptors (CARs) used in the Examples are provided in Tables 12 and 13.

TABLE 12
Amino acid sequences for chimeric antigen receptors (CARs) used in the
examples of the application.

| | CAR Domain | |
|---|---|---|
| | (listed from | |
| | top-to-bottom | |
| CAR | in order from | |
| Polypeptide | N-terminus to | |
| Description | C-terminus) | CAR Domain Amino Acid Sequence |
| Anti- | CD8a Leader | MALPVTALLLPLALLLHAARP (SEQ ID NO: 836) |
| CD19 | Peptide | |
| CAR | scFv | DIQMTQTTSSLSASLGDRVTISCRASQDISKYLNWYQQKPDGTVKL |
| with | | LIYHTSRLHSGVPSRFSGSGSGTDYSLTISNLEQEDIATYFCQQGN |
| CD28 | | TLPYTFGGGTKLEITGGGGSGGGGSGGGGSEVKLQESGPGLVAPSQ |
| co- | | SLSVTCTVSGVSLPDYGVSWIRQPPRKGLEWLGVIWGSETTYYNSA |
| stim | | LKSRLTIIKDNSKSQVFLKMNSLQTDDTAIYYCAKHYYYGGSYAMD |
| | | YWGQGTSVTVSS (SEQ ID NO: 837) |
| | CD8a Hinge | TTTPAPRPPTPAPTIASQPLSLRPEACRPAAGGAVHTRGLDFACD |
| | | (SEQ ID NO: 838) |
| | CD28 | FWVLVVVGGVLACYSLLVTVAFIIFWV (SEQ ID NO: 839) |
| | Transmembrane | |
| | CD28 | RSKRSRLLHSDYMNMTPRRPGPTRKHYQPYAPPRDFAAYRS (SEQ |
| | Intracellular | ID NO: 840) |
| | CD3 zeta | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR (SEQ ID NO: 841) |
| | tEGFR Tag | MALPVTALLLPLALLLHAARPGSRKVCNGIGIGEFKDSLSINATNI |
| | | KHFKNCTSISGDLHILPVAFRGDSFTHTPPLDPQELDILKTVKEIT |
| | | GFLLIQAWPENRTDLHAFENLEIIRGRTKQHGQFSLAVVSLNITSL |
| | | GLRSLKEISDGDVIISGNKNLCYANTINWKKLFGTSGQKTKIISNR |
| | | GENSCKATGQVCHALCSPEGCWGPEPRDCVSCRNVSRGRECVDKCN |
| | | LLEGEPREFVENSECIQCHPECLPQAMNITCTGRGPDNCIQCAHYI |
| | | DGPHCVKTCPAGVMGENNTLVWKYADAGHVCHLCHPNCTYGCTGPG |
| | | LEGCPTNGPKIPSIATGMVGALLLLLVVALGIGLFM (SEQ ID NO: |
| | | 842) |
| Anti- | CD8a Leader | MALPVTALLLPLALLLHAARP (SEQ ID NO: 843) |
| CD22 | Peptide | |
| CAR | scFv | QVQLQQSGPGLVKPSQTLSLTCAISGDSVSSNSAAWNWIRQSPSRG |
| | | LEWLGRTYYRSKWYNDYAVSVKSRITINPDTSKNQFSLQLNSVTPE |
| | | DTAVYYCAREVTGDLEDAFDIWGQGTMVTVSSGGGGSDIQMTQSPS |
| | | SLSASVGDRVTITCRASQTIWSYLNWYQQRPGKAPNLLIYAASSLQ |
| | | SGVPSRFSGRGSGTDFTLTISSLQAEDFATYYCQQSYSIPQTFGQG |
| | | TKLEIK (SEQ ID NO: 844) |
| | CD8a Hinge | TTTPAPRPPTPAPTIASQPLSLRPEACRPAAGGAVHTRGLDFACD |
| | | (SEQ ID NO: 845) |
| | CD8a | IYIWAPLAGTCGVLLLSLVITLYC (SEQ ID NO: 846) |
| | Transmembrane | |
| | 4-1BB | KRGRKKLLYIFKQPFMRPVQTTQEEDGCSCRFPEEEEGGCEL (SEQ |
| | Intracellular | ID NO: 847) |
| | CD3 zeta | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR (SEQ ID NO: 848) |
| | tEGFR Tag | MALPVTALLLPLALLLHAARPGSRKVCNGIGIGEFKDSLSINATNI |
| | | KHFKNCTSISGDLHILPVAFRGDSFTHTPPLDPQELDILKTVKEIT |
| | | GFLLIQAWPENRTDLHAFENLEIIRGRTKQHGQFSLAVVSLNITSL |
| | | GLRSLKEISDGDVIISGNKNLCYANTINWKKLFGTSGQKTKIISNR |
| | | GENSCKATGQVCHALCSPEGCWGPEPRDCVSCRNVSRGRECVDKCN |
| | | LLEGEPREFVENSECIQCHPECLPQAMNITCTGRGPDNCIQCAHYI |
| | | DGPHCVKTCPAGVMGENNTLVWKYADAGHVCHLCHPNCTYGCTGPG |
| | | LEGCPTNGPKIPSIATGMVGALLLLLVVALGIGLFM (SEQ ID NO: |
| | | 849) |
| Anti- | Signal Peptide | MLLLVTSLLLCELPHPAFLLIP (SEQ ID NO: 850) |
| ROR1 | scFv | QEQLVESGGRLVTPGGSLTLSCKASGFDFSAYYMSWVRQAPGKGLE |
| CAR | | WIATIYPSSGKTYYATWVNGRFTISSDNAQNTVDLQMNSLTAADRA |
| | | TYFCARDSYADDGALFNIWGPGTLVTISSGGGGSGGGGSGGGGSEL |
| | | VLTQSPSVSAALGSPAKITCTLSSAHKTDTIDWYQQLQGEAPRYLM |
| | | QVQSDGSYTKRPGVPDRFSGSSSGADRYLIIPSVQADDEADYYCGA |
| | | DYIGGYVFGGGTQLTVTG (SEQ ID NO: 851) |
| | CD8a | ESKYGPPCPPCPMFWVLVVVGGVLACYSLLVTVAFIIFWV (SEQ ID |
| | Hinge/ | NO: 852) |
| | Transmembrane | |
| | CD28 | RSKRSRLLHSDYMNMTPRRPGPTRKHYQPYAPPRDFAAYRS (SEQ |
| | Intracellular | ID NO: 853) |
| | CD3 zeta | RVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQG |
| | | LSTATKDTYDALHMQALPPR (SEQ ID NO: 854) |
| | tEGFR Tag | MLLLVTSLLLCELPHPAFLLIPRKVCNGIGIGEFKDSLSINATNIK |
| | | HFKNCTSISGDLHILPVAFRGDSFTHTPPLDPQELDILKTVKEITG |
| | | FLLIQAWPENRTDLHAFENLEIIRGRTKQHGQFSLAVVSLNITSLG |
| | | LRSLKEISDGDVIISGNKNLCYANTINWKKLFGTSGQKTKIISNRG |
| | | ENSCKATGQVCHALCSPEGCWGPEPRDCVSCRNVSRGRECVDKCNL |
| | | LEGEPREFVENSECIQCHPECLPQAMNITCTGRGPDNCIQCAHYID |
| | | GPHCVKTCPAGVMGENNTLVWKYADAGHVCHLCHPNCTYGCTGPGL |
| | | EGCPTNGPKIPSIATGMVGALLLLLVVALGIGLFM (SEQ ID NO: |
| | | 855) |

TABLE 13
Nucleotide sequences for chimeric antigen receptors (CARs) used in the
examples of the application.

| | CAR Domain (listed | |
|---|---|---|
| CAR | from top-to-bottom in | |
| Polypeptide | order from 5′ end to | |
| Description | 3′ end) | Nucleotide Sequence |
| Anti-CD19 | CD8a Leader Peptide | ATGGCCCTGCCCGTGACCGCCCTGCTGCTGCCCCTGGCCC |
| CAR with | scFv | TGCTGCTGCACGCCGCCAGGCCC (SEQ ID NO: 856) |
| CD28 co- | | GACATCCAGATGACCCAGACTACCAGCTCCCTGAGCGCCA |
| stim | | GTCTGGGCGATCGGGTGACCATCAGCTGCAGGGCCAGCCA |
| | | GGACATCTCCAAGTACCTGAATTGGTACCAACAGAAACCC |
| | | GACGGCACCGTGAAGCTGCTGATCTACCACACCTCTAGGC |
| | | TGCACAGCGGGGTGCCCAGCCGCTTCAGCGGAAGTGGCAG |
| | | TGGCACCGACTACTCCCTGACCATCAGCAACCTGGAGCAG |
| | | GAGGACATTGCCACCTACTTCTGCCAGCAGGGCAACACCC |
| | | TGCCCTACACCTTCGGCGGCGGCACCAAGCTGGAGATCAC |
| | | AGGCGGGGGCGGCTCTGGCGGGGGCGGCAGCGGGGGCGGG |
| | | GGCTCCGAGGTGAAGCTGCAGGAGTCCGGCCCCGGCCTGG |
| | | TGGCCCCCAGCCAGAGCCTGTCCGTGACCTGTACCGTGTC |
| | | CGGGGTGTCCCTGCCCGACTATGGCGTGAGCTGGATCCGC |
| | | CAGCCACCCAGGAAGGGCCTGGAGTGGCTGGGCGTGATCT |
| | | GGGGCAGCGAGACCACCTACTACAATAGCGCCCTGAAGAG |
| | | CAGGCTGACCATCATCAAGGACAACAGCAAGAGCCAGGTG |
| | | TTCCTGAAGATGAACAGCCTGCAGACCGACGACACCGCCA |
| | | TCTACTACTGTGCCAAGCACTACTACTATGGCGGCAGCTA |
| | | CGCCATGGACTACTGGGGGCAGGGCACCTCCGTGACCGTG |
| | | AGTAGC (SEQ ID NO: 857) |
| | CD8a Hinge | ACCACCACCCCAGCACCCCGCCCACCCACCCCAGCCCCAA |
| | | CAATCGCAAGCCAGCCCCTGTCCCTGCGGCCCGAGGCCTG |
| | | CCGGCCCGCCGCCGGCGGCGCCGTGCACACCAGGGGCCTG |
| | | GACTTCGCTTGCGAT (SEQ ID NO: 858) |
| | CD28 Transmembrane | TTTTGGGTGCTGGTGGTGGTGGGCGGGGTGCTGGCCTGCT |
| | | ACTCCCTGCTGGTGACCGTGGCCTTCATCATCTTCTGGGT |
| | | G (SEQ ID NO: 859) |
| | CD28 Intracellular | CGCTCCAAGCGGAGCAGGCTGCTGCACAGCGACTACATGA |
| | | ACATGACACCCAGAAGACCCGGCCCCACACGGAAGCATTA |
| | | CCAGCCCTACGCCCCTCCCCGCGACTTCGCCGCCTACCGG |
| | | AGC (SEQ ID NO: 860) |
| | CD3 zeta | CGGGTGAAGTTCAGCCGCTCCGCCGATGCCCCTGCCTACC |
| | | AGCAGGGGCAGAATCAGCTGTACAACGAGCTGAACCTGGG |
| | | CCGGCGCGAGGAGTACGACGTGCTGGACAAGCGCCGGGGC |
| | | CGCGACCCAGAGATGGGCGGCAAGCCCCGGCGGAAGAATC |
| | | CCCAGGAGGGCCTGTACAACGAGCTCCAGAAGGACAAGAT |
| | | GGCCGAGGCCTACAGCGAGATCGGCATGAAGGGCGAGCGG |
| | | AGACGCGGGAAGGGGCACGACGGCCTGTACCAGGGCCTGT |
| | | CCACCGCCACCAAGGACACCTACGACGCCCTGCACATGCA |
| | | GGCCCTGCCCCCCCGC (SEQ ID NO: 861) |
| | tEGFR Tag | ATGGCCCTGCCTGTGACAGCTCTGCTGCTTCCTCTGGCAC |
| | | TGCTGCTGCATGCTGCCAGACCAGGCAGCAGAAAAGTGTG |
| | | CAACGGCATCGGCATCGGAGAGTTCAAGGACAGCCTGAGC |
| | | ATCAACGCCACCAACATCAAGCACTTCAAGAACTGCACCA |
| | | GCATCAGCGGCGACCTGCACATTCTGCCTGTGGCCTTTAG |
| | | AGGCGACAGCTTCACCCACACACCTCCACTCGATCCCCAG |
| | | GAGCTGGACATCCTGAAAACCGTGAAAGAGATCACCGGCT |
| | | TTCTGCTGATCCAGGCTTGGCCCGAGAACCGGACAGATCT |
| | | GCACGCCTTCGAGAACCTGGAAATCATCAGAGGCCGGACC |
| | | AAGCAGCACGGCCAGTTTTCTCTGGCTGTGGTGTCCCTGA |
| | | ACATCACCAGCCTGGGCCTGAGAAGCCTGAAAGAAATCAG |
| | | CGACGGCGACGTGATCATCTCCGGCAACAAGAACCTGTGC |
| | | TACGCCAACACCATCAACTGGAAGAAGCTGTTCGGCACCA |
| | | GCGGCCAGAAAACAAAGATCATCAGCAACCGGGGCGAGAA |
| | | CAGCTGCAAGGCTACAGGCCAAGTGTGCCACGCTCTGTGT |
| | | AGCCCTGAAGGCTGTTGGGGACCCGAGCCTAGAGATTGCG |
| | | TGTCCTGCAGAAACGTGTCCCGGGGCAGAGAATGCGTGGA |
| | | CAAGTGCAATCTGCTGGAAGGCGAGCCCCGCGAGTTCGTG |
| | | GAAAACAGCGAGTGCATCCAGTGTCACCCCGAGTGTCTGC |
| | | CCCAGGCCATGAACATTACCTGTACCGGCAGAGGCCCCGA |
| | | CAACTGTATTCAGTGCGCCCACTACATCGACGGCCCTCAC |
| | | TGCGTGAAAACATGTCCTGCTGGCGTGATGGGAGAGAACA |
| | | ACACCCTCGTGTGGAAGTATGCCGACGCCGGACATGTGTG |
| | | CCACCTGTGTCACCCTAATTGCACCTACGGCTGTACAGGC |
| | | CCTGGCCTGGAAGGCTGTCCAACAAACGGACCTAAGATCC |
| | | CCTCTATCGCCACCGGCATGGTTGGAGCCTTGCTGCTTCT |
| | | GCTGGTGGTGGCCCTTGGCATCGGCCTGTTTATG (SEQ |
| | | ID NO: 862) |
| Anti-CD22 | CD8a Leader Peptide | ATGGCCCTTCCAGTAACTGCCCTTCTGTTGCCACTTGCTC |
| CAR | scFv | TTCTCCTTCACGCGGCGCGCCCA (SEQ ID NO: 863) |
| | | CAGGTGCAGCTGCAGCAGAGCGGCCCGGGCCTGGTGAAAC |
| | | CGAGCCAGACCCTGAGCCTGACCTGCGCGATTAGCGGCGA |
| | | TAGCGTGAGCAGCAACAGCGCGGCGTGGAACTGGATTCGC |
| | | CAGAGCCCGAGCCGCGGCCTGGAATGGCTGGGCCGCACCT |
| | | ATTATCGCAGCAAATGGTATAACGATTATGCGGTGAGCGT |
| | | GAAAAGCCGCATTACCATTAACCCGGATACCAGCAAAAAC |
| | | CAGTTTAGCCTGCAGCTGAACAGCGTGACCCCGGAAGATA |
| | | CCGCGGTGTATTATTGCGCGCGCGAAGTGACCGGCGATCT |
| | | GGAAGATGCGTTTGATATTTGGGGCCAGGGCACCATGGTG |
| | | ACCGTGTCGTCCGGCGGAGGCGGGTCAGATATTCAGATGA |
| | | CCCAGTCACCCAGTTCACTCTCTGCGTCCGTTGGAGATAG |
| | | AGTCACAATTACATGTCGCGCGAGCCAGACGATTTGGAGT |
| | | TACCTCAATTGGTATCAGCAGCGCCCGGGCAAAGCGCCGA |
| | | ACCTGCTGATTTATGCGGCGAGCAGCCTGCAGAGCGGCGT |
| | | GCCGAGCCGCTTTAGCGGCCGCGGCAGCGGCACCGATTTT |
| | | ACCCTGACCATTAGCAGCCTGCAGGCGGAAGATTTTGCGA |
| | | CCTATTATTGCCAGCAGAGCTATAGCATTCCGCAGACCTT |
| | | TGGCCAGGGCACCAAACTGGAAATTAAA (SEQ ID NO: |
| | | 864) |
| | CD8a Hinge | ACGACTACACCAGCTCCACGCCCGCCAACTCCAGCACCTA |
| | | CAATTGCATCACAACCTCTGAGCTTGAGGCCTGAGGCATG |
| | | TAGACCTGCCGCTGGAGGTGCTGTGCATACACGCGGACTG |
| | | GATTTTGCTTGCGAT (SEQ ID NO: 865) |
| | CD8a Transmembrane | ATTTATATTTGGGCCCCATTGGCTGGGACCTGCGGCGTCT |
| | | TGCTCCTGTCTCTTGTCATTACTTTGTATTGC (SEQ ID |
| | | NO: 866) |
| | 4-1BB Intracellular | AAGCGCGGAAGGAAAAAGTTGTTGTATATCTTTAAGCAAC |
| | | CCTTCATGCGCCCGGTTCAGACAACACAGGAAGAGGACGG |
| | | ATGCTCTTGTAGGTTTCCTGAGGAAGAAGAGGGCGGGTGC |
| | | GAACTC (SEQ ID NO: 867) |
| | CD3 zeta | AGAGTCAAATTTTCACGCTCCGCCGATGCACCTGCATACC |
| | | AGCAAGGGCAAAATCAACTGTACAACGAACTGAACCTCGG |
| | | CAGGCGGGAAGAATACGACGTGCTCGACAAACGACGCGGG |
| | | AGAGACCCCGAAATGGGTGGCAAACCCCGCCGGAAAAATC |
| | | CACAGGAGGGCTTGTATAACGAACTTCAAAAGGACAAGAT |
| | | GGCTGAAGCTTACTCAGAAATCGGTATGAAGGGAGAACGG |
| | | AGGCGCGGTAAAGGCCACGACGGATTGTACCAAGGACTGT |
| | | CTACCGCTACAAAAGACACTTACGATGCACTGCATATGCA |
| | | AGCACTCCCGCCGAGA (SEQ ID NO: 868) |
| | tEGFR Tag | ATGGCCCTGCCTGTGACAGCTCTGCTGCTTCCTCTGGCAC |
| | | TGCTGCTGCATGCTGCCAGACCAGGCAGCAGAAAAGTGTG |
| | | CAACGGCATCGGCATCGGAGAGTTCAAGGACAGCCTGAGC |
| | | ATCAACGCCACCAACATCAAGCACTTCAAGAACTGCACCA |
| | | GCATCAGCGGCGACCTGCACATTCTGCCTGTGGCCTTTAG |
| | | AGGCGACAGCTTCACCCACACACCTCCACTCGATCCCCAG |
| | | GAGCTGGACATCCTGAAAACCGTGAAAGAGATCACCGGCT |
| | | TTCTGCTGATCCAGGCTTGGCCCGAGAACCGGACAGATCT |
| | | GCACGCCTTCGAGAACCTGGAAATCATCAGAGGCCGGACC |
| | | AAGCAGCACGGCCAGTTTTCTCTGGCTGTGGTGTCCCTGA |
| | | ACATCACCAGCCTGGGCCTGAGAAGCCTGAAAGAAATCAG |
| | | CGACGGCGACGTGATCATCTCCGGCAACAAGAACCTGTGC |
| | | TACGCCAACACCATCAACTGGAAGAAGCTGTTCGGCACCA |
| | | GCGGCCAGAAAACAAAGATCATCAGCAACCGGGGCGAGAA |
| | | CAGCTGCAAGGCTACAGGCCAAGTGTGCCACGCTCTGTGT |
| | | AGCCCTGAAGGCTGTTGGGGACCCGAGCCTAGAGATTGCG |
| | | TGTCCTGCAGAAACGTGTCCCGGGGCAGAGAATGCGTGGA |
| | | CAAGTGCAATCTGCTGGAAGGCGAGCCCCGCGAGTTCGTG |
| | | GAAAACAGCGAGTGCATCCAGTGTCACCCCGAGTGTCTGC |
| | | CCCAGGCCATGAACATTACCTGTACCGGCAGAGGCCCCGA |
| | | CAACTGTATTCAGTGCGCCCACTACATCGACGGCCCTCAC |
| | | TGCGTGAAAACATGTCCTGCTGGCGTGATGGGAGAGAACA |
| | | ACACCCTCGTGTGGAAGTATGCCGACGCCGGACATGTGTG |
| | | CCACCTGTGTCACCCTAATTGCACCTACGGCTGTACAGGC |
| | | CCTGGCCTGGAAGGCTGTCCAACAAACGGACCTAAGATCC |
| | | CCTCTATCGCCACCGGCATGGTTGGAGCCTTGCTGCTTCT |
| | | GCTGGTGGTGGCCCTTGGCATCGGCCTGTTTATG (SEQ |
| | | ID NO: 869) |
| Anti-ROR1 | Signal Peptide | ATGCTGCTGCTGGTGACAAGCCTGCTGCTGTGCGAGCTGC |
| CAR | scFv | CCCACCCCGCCTTTCTGCTGATCCCC (SEQ ID NO: 870) |
| | | CAGGAACAGCTCGTCGAAAGCGGCGGCAGACTGGTGACAC |
| | | CTGGCGGCAGCCTGACCCTGAGCTGCAAGGCCAGCGGCTT |
| | | CGACTTCAGCGCCTACTACATGAGCTGGGTCCGCCAGGCC |
| | | CCTGGCAAGGGACTGGAATGGATCGCCACCATCTACCCCA |
| | | GCAGCGGCAAGACCTACTACGCCACCTGGGTGAACGGACG |
| | | GTTCACCATCTCCAGCGACAACGCCCAGAACACCGTGGAC |
| | | CTGCAGATGAACAGCCTGACAGCCGCCGACCGGGCCACCT |
| | | ACTTTTGCGCCAGAGACAGCTACGCCGACGACGGCGCCCT |
| | | GTTCAACATCTGGGGCCCTGGCACCCTGGTGACAATCTCT |
| | | AGCGGCGGAGGCGGATCTGGTGGCGGAGGAAGTGGCGGCG |
| | | GAGGATCTGAGCTGGTGCTGACCCAGAGCCCCTCTGTGTC |
| | | TGCTGCCCTGGGAAGCCCTGCCAAGATCACCTGTACCCTG |
| | | AGCAGCGCCCACAAGACCGACACCATCGACTGGTATCAGC |
| | | AGCTGCAGGGCGAGGCCCCCAGATACCTGATGCAGGTGCA |
| | | GAGCGACGGCAGCTACACCAAGAGGCCAGGCGTGCCCGAC |
| | | CGGTTCAGCGGATCTAGCTCTGGCGCCGACCGCTACCTGA |
| | | TCATCCCCAGCGTGCAGGCCGATGACGAGGCCGATTACTA |
| | | CTGTGGCGCCGACTACATCGGCGGCTACGTGTTCGGCGGA |
| | | GGCACCCAGCTGACCGTGACCGGC (SEQ ID NO: 871) |
| | CD8a | GAGTCTAAGTACGGACCGCCCTGCCCCCCTTGCCCTATGT |
| | Hinge/Transmembrane | TCTGGGTGCTGGTGGTGGTGGGCGGGGTGCTGGCCTGCTA |
| | | CAGCCTGCTGGTGACAGTGGCCTTCATCATCTTTTGGGTG |
| | | (SEQ ID NO: 872) |
| | CD28 Intracellular | CGGAGCAAGCGGAGCAGGCTGCTGCATTCGGATTACATGA |
| | | ACATGACCCCAAGGAGGCCTGGTCCGACTCGGAAGCACTA |
| | | CCAGCCCTATGCCCCGCCTCGCGATTTCGCCGCCTACCGC |
| | | TCA (SEQ ID NO: 873) |
| | CD3 zeta | CGGGTGAAGTTCAGCAGAAGCGCCGACGCCCCTGCCTACC |
| | | AGCAGGGCCAGAATCAGCTGTACAACGAGCTGAACCTGGG |
| | | CAGAAGGGAAGAGTACGACGTCCTGGATAAGCGGAGAGGC |
| | | CGGGACCCTGAGATGGGCGGCAAGCCTCGGCGGAAGAACC |
| | | CCCAGGAAGGCCTGTATAACGAACTGCAGAAAGACAAGAT |
| | | GGCCGAGGCCTACAGCGAGATCGGCATGAAGGGCGAGCGG |
| | | AGGCGGGGCAAGGGCCACGACGGCCTGTATCAGGGCCTGT |
| | | CCACCGCCACCAAGGATACCTACGACGCCCTGCACATGCA |
| | | GGCCCTGCCCCCAAGG (SEQ ID NO: 874) |
| | tEGFR Tag | ATGCTTCTCCTGGTGACAAGCCTTCTGCTCTGTGAGTTAC |
| | | CACACCCAGCATTCCTCCTGATCCCACGCAAAGTGTGTAA |
| | | CGGAATAGGTATTGGTGAATTTAAAGACTCACTCTCCATA |
| | | AATGCTACGAATATTAAACACTTCAAAAACTGCACCTCCA |
| | | TCAGTGGCGATCTCCACATCCTGCCGGTGGCATTTAGGGG |
| | | TGACTCCTTCACACATACTCCTCCTCTGGATCCACAGGAA |
| | | CTGGATATTCTGAAAACCGTAAAGGAAATCACAGGGTTTT |
| | | TGCTGATTCAGGCTTGGCCTGAAAACAGGACGGACCTCCA |
| | | TGCCTTTGAGAACCTAGAAATCATACGCGGCAGGACCAAG |
| | | CAACATGGTCAGTTTTCTCTTGCAGTCGTCAGCCTGAACA |
| | | TAACATCCTTGGGATTACGCTCCCTCAAGGAGATAAGTGA |
| | | TGGAGATGTGATAATTTCAGGAAACAAAAATTTGTGCTAT |
| | | GCAAATACAATAAACTGGAAAAAACTGTTTGGGACCTCCG |
| | | GTCAGAAAACCAAAATTATAAGCAACAGAGGTGAAAACAG |
| | | CTGCAAGGCCACAGGCCAGGTCTGCCATGCCTTGTGCTCC |
| | | CCCGAGGGCTGCTGGGGCCCGGAGCCCAGGGACTGCGTCT |
| | | CTTGCCGGAATGTCAGCCGAGGCAGGGAATGCGTGGACAA |
| | | GTGCAACCTTCTGGAGGGTGAGCCAAGGGAGTTTGTGGAG |
| | | AACTCTGAGTGCATACAGTGCCACCCAGAGTGCCTGCCTC |
| | | AGGCCATGAACATCACCTGCACAGGACGGGGACCAGACAA |
| | | CTGTATCCAGTGTGCCCACTACATTGACGGCCCCCACTGC |
| | | GTCAAGACCTGCCCGGCAGGAGTCATGGGAGAAAACAACA |
| | | CCCTGGTCTGGAAGTACGCAGACGCCGGCCATGTGTGCCA |
| | | CCTGTGCCATCCAAACTGCACCTACGGATGCACTGGGCCA |
| | | GGTCTTGAAGGCTGTCCAACGAATGGGCCTAAGATCCCGT |
| | | CCATCGCCACTGGGATGGTGGGGGCCCTCCTCTTGCTGCT |
| | | GGTGGTGGCCCTGGGGATCGGCCTCTTCATGV (SEQ ID |
| | | NO: 875) |

TABLE 14
Prime editing guide RNA (pegRNA) sequences. In some embodiments, a
pegRNA sequences further contain a 5′ G. In some cases, a pegRNA further comprises
the sequence “cacc” or “caccg” at the 5′ end.

| | SEQ ID |
|---|---|
| pegRNA Sequence | NO |
| CCUAAAGUUAAUUAUGUACCguuuuagaGCUAGAAAUAGCAAGUUAAAAUAAGGCUAGUCCGU | 882 |
| UAUCAACUUGAAAAAGUGGCACCGAGUCGgugcUGGACCAGGCACAUAAUUAA | |
| CCUAAAGUUAAUUAUGUACCguuuuagaGCUAGAAAUAGCAAGUUAAAAUAAGGCUAGUCCGU | 883 |
| UAUCAACUUGAAAAAGUGGCACCGAGUCGgugcUGGACCAGGCACAUAAUUAAC | |
| CCUAAAGUUAAUUAUGUACCguuuuagaGCUAGAAAUAGCAAGUUAAAAUAAGGCUAGUCCGU | 884 |
| UAUCAACUUGAAAAAGUGGCACCGAGUCGgugcUGGACCAGGCACAUAAUUAACU | |
| CCUAAAGUUAAUUAUGUACCguuuuagaGCUAGAAAUAGCAAGUUAAAAUAAGGCUAGUCCGU | 885 |
| UAUCAACUUGAAAAAGUGGCACCGAGUCGgugcUGGACCAGGCACAUAAUUAACUU | |
| CCUAAAGUUAAUUAUGUACCguuuuagaGCUAGAAAUAGCAAGUUAAAAUAAGGCUAGUCCGU | 886 |
| UAUCAACUUGAAAAAGUGGCACCGAGUCGgugcUGGACCAGGCACAUAAUUAACUUU | |
| CCUAAAGUUAAUUAUGUACCguuuuagaGCUAGAAAUAGCAAGUUAAAAUAAGGCUAGUCCGU | 887 |
| UAUCAACUUGAAAAAGUGGCACCGAGUCGgugcUGGACCAGGCACAUAAUUAACUUUA | |
| CCUAAAGUUAAUUAUGUACCguuuuagaGCUAGAAAUAGCAAGUUAAAAUAAGGCUAGUCCGU | 888 |
| UAUCAACUUGAAAAAGUGGCACCGAGUCGgugcUUGGACCAGGCACAUAAUUAA | |
| CCUAAAGUUAAUUAUGUACCguuuuagaGCUAGAAAUAGCAAGUUAAAAUAAGGCUAGUCCGU | 889 |
| UAUCAACUUGAAAAAGUGGCACCGAGUCGgugcUUGGACCAGGCACAUAAUUAAC | |
| CCUAAAGUUAAUUAUGUACCguuuuagaGCUAGAAAUAGCAAGUUAAAAUAAGGCUAGUCCGU | 890 |
| UAUCAACUUGAAAAAGUGGCACCGAGUCGgugcUUGGACCAGGCACAUAAUUAACU | |
| CCUAAAGUUAAUUAUGUACCguuuuagaGCUAGAAAUAGCAAGUUAAAAUAAGGCUAGUCCGU | 891 |
| UAUCAACUUGAAAAAGUGGCACCGAGUCGgugcUUGGACCAGGCACAUAAUUAACUU | |
| CCUAAAGUUAAUUAUGUACCguuuuagaGCUAGAAAUAGCAAGUUAAAAUAAGGCUAGUCCGU | 892 |
| UAUCAACUUGAAAAAGUGGCACCGAGUCGgugcUUGGACCAGGCACAUAAUUAACUUU | |
| CCUAAAGUUAAUUAUGUACCguuuuagaGCUAGAAAUAGCAAGUUAAAAUAAGGCUAGUCCGU | 893 |
| UAUCAACUUGAAAAAGUGGCACCGAGUCGgugcUUGGACCAGGCACAUAAUUAACUUUA | |
| CCUAAAGUUAAUUAUGUACCguuuuagaGCUAGAAAUAGCAAGUUAAAAUAAGGCUAGUCCGU | 894 |
| UAUCAACUUGAAAAAGUGGCACCGAGUCGgugcCUUGGACCAGGCACAUAAUUAA | |
| CCUAAAGUUAAUUAUGUACCguuuuagaGCUAGAAAUAGCAAGUUAAAAUAAGGCUAGUCCGU | 895 |
| UAUCAACUUGAAAAAGUGGCACCGAGUCGgugcCUUGGACCAGGCACAUAAUUAAC | |
| CCUAAAGUUAAUUAUGUACCguuuuagaGCUAGAAAUAGCAAGUUAAAAUAAGGCUAGUCCGU | 896 |
| UAUCAACUUGAAAAAGUGGCACCGAGUCGgugcCUUGGACCAGGCACAUAAUUAACU | |
| CCUAAAGUUAAUUAUGUACCguuuuagaGCUAGAAAUAGCAAGUUAAAAUAAGGCUAGUCCGU | 897 |
| UAUCAACUUGAAAAAGUGGCACCGAGUCGgugcCUUGGACCAGGCACAUAAUUAACUU | |
| CCUAAAGUUAAUUAUGUACCguuuuagaGCUAGAAAUAGCAAGUUAAAAUAAGGCUAGUCCGU | 898 |
| UAUCAACUUGAAAAAGUGGCACCGAGUCGgugcCUUGGACCAGGCACAUAAUUAACUUU | |
| CCUAAAGUUAAUUAUGUACCguuuuagaGCUAGAAAUAGCAAGUUAAAAUAAGGCUAGUCCGU | 899 |
| UAUCAACUUGAAAAAGUGGCACCGAGUCGgugcCUUGGACCAGGCACAUAAUUAACUUUA | |
| CCUAAAGUUAAUUAUGUACCguuuuagaGCUAGAAAUAGCAAGUUAAAAUAAGGCUAGUCCGU | 900 |
| UAUCAACUUGAAAAAGUGGCACCGAGUCGgugcCCUUGGACCAGGCACAUAAUUAA | |
| CCUAAAGUUAAUUAUGUACCguuuuagaGCUAGAAAUAGCAAGUUAAAAUAAGGCUAGUCCGU | 901 |
| UAUCAACUUGAAAAAGUGGCACCGAGUCGgugcCCUUGGACCAGGCACAUAAUUAAC | |
| CCUAAAGUUAAUUAUGUACCguuuuagaGCUAGAAAUAGCAAGUUAAAAUAAGGCUAGUCCGU | 902 |
| UAUCAACUUGAAAAAGUGGCACCGAGUCGgugcCCUUGGACCAGGCACAUAAUUAACU | |
| CCUAAAGUUAAUUAUGUACCguuuuagaGCUAGAAAUAGCAAGUUAAAAUAAGGCUAGUCCGU | 903 |
| UAUCAACUUGAAAAAGUGGCACCGAGUCGgugcCCUUGGACCAGGCACAUAAUUAACUU | |
| CCUAAAGUUAAUUAUGUACCguuuuagaGCUAGAAAUAGCAAGUUAAAAUAAGGCUAGUCCGU | 904 |
| UAUCAACUUGAAAAAGUGGCACCGAGUCGgugcCCUUGGACCAGGCACAUAAUUAACUUU | |
| CCUAAAGUUAAUUAUGUACCguuuuagaGCUAGAAAUAGCAAGUUAAAAUAAGGCUAGUCCGU | 905 |
| UAUCAACUUGAAAAAGUGGCACCGAGUCGgugcCCUUGGACCAGGCACAUAAUUAACUUUA | |
| CCUAAAGUUAAUUAUGUACCguuuuagaGCUAGAAAUAGCAAGUUAAAAUAAGGCUAGUCCGU | 906 |
| UAUCAACUUGAAAAAGUGGCACCGAGUCGgugcUCCUUGGACCAGGCACAUAAUUAA | |
| CCUAAAGUUAAUUAUGUACCguuuuagaGCUAGAAAUAGCAAGUUAAAAUAAGGCUAGUCCGU | 907 |
| UAUCAACUUGAAAAAGUGGCACCGAGUCGgugcUCCUUGGACCAGGCACAUAAUUAAC | |
| CCUAAAGUUAAUUAUGUACCguuuuagaGCUAGAAAUAGCAAGUUAAAAUAAGGCUAGUCCGU | 908 |
| UAUCAACUUGAAAAAGUGGCACCGAGUCGgugcUCCUUGGACCAGGCACAUAAUUAACU | |
| CCUAAAGUUAAUUAUGUACCguuuuagaGCUAGAAAUAGCAAGUUAAAAUAAGGCUAGUCCGU | 909 |
| UAUCAACUUGAAAAAGUGGCACCGAGUCGgugcUCCUUGGACCAGGCACAUAAUUAACUU | |
| CCUAAAGUUAAUUAUGUACCguuuuagaGCUAGAAAUAGCAAGUUAAAAUAAGGCUAGUCCGU | 910 |
| UAUCAACUUGAAAAAGUGGCACCGAGUCGgugcUCCUUGGACCAGGCACAUAAUUAACUUU | |
| CCUAAAGUUAAUUAUGUACCguuuuagaGCUAGAAAUAGCAAGUUAAAAUAAGGCUAGUCCGU | 911 |
| UAUCAACUUGAAAAAGUGGCACCGAGUCGgugcUCCUUGGACCAGGCACAUAAUUAACUUUA | |
| CCUAAAGUUAAUUAUGUACCguuuuagaGCUAGAAAUAGCAAGUUAAAAUAAGGCUAGUCCGU | 912 |
| UAUCAACUUGAAAAAGUGGCACCGAGUCGgugcUUCCUUGGACCAGGCACAUAAUUAA | |
| CCUAAAGUUAAUUAUGUACCguuuuagaGCUAGAAAUAGCAAGUUAAAAUAAGGCUAGUCCGU | 913 |
| UAUCAACUUGAAAAAGUGGCACCGAGUCGgugcUUCCUUGGACCAGGCACAUAAUUAAC | |
| CCUAAAGUUAAUUAUGUACCguuuuagaGCUAGAAAUAGCAAGUUAAAAUAAGGCUAGUCCGU | 914 |
| UAUCAACUUGAAAAAGUGGCACCGAGUCGgugcUUCCUUGGACCAGGCACAUAAUUAACU | |
| CCUAAAGUUAAUUAUGUACCguuuuagaGCUAGAAAUAGCAAGUUAAAAUAAGGCUAGUCCGU | 915 |
| UAUCAACUUGAAAAAGUGGCACCGAGUCGgugcUUCCUUGGACCAGGCACAUAAUUAACUU | |
| CCUAAAGUUAAUUAUGUACCguuuuagaGCUAGAAAUAGCAAGUUAAAAUAAGGCUAGUCCGU | 916 |
| UAUCAACUUGAAAAAGUGGCACCGAGUCGgugcUUCCUUGGACCAGGCACAUAAUUAACUUU | |
| CCUAAAGUUAAUUAUGUACCguuuuagaGCUAGAAAUAGCAAGUUAAAAUAAGGCUAGUCCGU | 917 |
| UAUCAACUUGAAAAAGUGGCACCGAGUCGgugcUUCCUUGGACCAGGCACAUAAUUAACUUUA | |
| CCUAAAGUUAAUUAUGUACCguuuuagaGCUAGAAAUAGCAAGUUAAAAUAAGGCUAGUCCGU | 918 |
| UAUCAACUUGAAAAAGUGGCACCGAGUCGgugcGUUCCUUGGACCAGGCACAUAAUUAA | |
| CCUAAAGUUAAUUAUGUACCguuuuagaGCUAGAAAUAGCAAGUUAAAAUAAGGCUAGUCCGU | 919 |
| UAUCAACUUGAAAAAGUGGCACCGAGUCGgugcGUUCCUUGGACCAGGCACAUAAUUAAC | |
| CCUAAAGUUAAUUAUGUACCguuuuagaGCUAGAAAUAGCAAGUUAAAAUAAGGCUAGUCCGU | 920 |
| UAUCAACUUGAAAAAGUGGCACCGAGUCGgugcGUUCCUUGGACCAGGCACAUAAUUAACU | |
| CCUAAAGUUAAUUAUGUACCguuuuagaGCUAGAAAUAGCAAGUUAAAAUAAGGCUAGUCCGU | 921 |
| UAUCAACUUGAAAAAGUGGCACCGAGUCGgugcGUUCCUUGGACCAGGCACAUAAUUAACUU | |
| CCUAAAGUUAAUUAUGUACCguuuuagaGCUAGAAAUAGCAAGUUAAAAUAAGGCUAGUCCGU | 922 |
| UAUCAACUUGAAAAAGUGGCACCGAGUCGgugcGUUCCUUGGACCAGGCACAUAAUUAACUUU | |
| CCUAAAGUUAAUUAUGUACCguuuuagaGCUAGAAAUAGCAAGUUAAAAUAAGGCUAGUCCGU | 923 |
| UAUCAACUUGAAAAAGUGGCACCGAGUCGgugcGUUCCUUGGACCAGGCACAUAAUUAACUUU | |
| A | |
| CCUAAAGUUAAUUAUGUACCguuuuagaGCUAGAAAUAGCAAGUUAAAAUAAGGCUAGUCCGU | 924 |
| UAUCAACUUGAAAAAGUGGCACCGAGUCGgugcAGUUCCUUGGACCAGGCACAUAAUUAA | |
| CCUAAAGUUAAUUAUGUACCguuuuagaGCUAGAAAUAGCAAGUUAAAAUAAGGCUAGUCCGU | 925 |
| UAUCAACUUGAAAAAGUGGCACCGAGUCGgugcAGUUCCUUGGACCAGGCACAUAAUUAAC | |
| CCUAAAGUUAAUUAUGUACCguuuuagaGCUAGAAAUAGCAAGUUAAAAUAAGGCUAGUCCGU | 926 |
| UAUCAACUUGAAAAAGUGGCACCGAGUCGgugcAGUUCCUUGGACCAGGCACAUAAUUAACU | |
| CCUAAAGUUAAUUAUGUACCguuuuagaGCUAGAAAUAGCAAGUUAAAAUAAGGCUAGUCCGU | 927 |
| UAUCAACUUGAAAAAGUGGCACCGAGUCGgugcAGUUCCUUGGACCAGGCACAUAAUUAACUU | |
| CCUAAAGUUAAUUAUGUACCguuuuagaGCUAGAAAUAGCAAGUUAAAAUAAGGCUAGUCCGU | 928 |
| UAUCAACUUGAAAAAGUGGCACCGAGUCGgugcAGUUCCUUGGACCAGGCACAUAAUUAACUU | |
| U | |
| CCUAAAGUUAAUUAUGUACCguuuuagaGCUAGAAAUAGCAAGUUAAAAUAAGGCUAGUCCGU | 929 |
| UAUCAACUUGAAAAAGUGGCACCGAGUCGgugcAGUUCCUUGGACCAGGCACAUAAUUAACUU | |
| UA | |
| CCUAAAGUUAAUUAUGUACCguuuuagaGCUAGAAAUAGCAAGUUAAAAUAAGGCUAGUCCGU | 930 |
| UAUCAACUUGAAAAAGUGGCACCGAGUCGgugcCAGUUCCUUGGACCAGGCACAUAAUUAA | |
| CCUAAAGUUAAUUAUGUACCguuuuagaGCUAGAAAUAGCAAGUUAAAAUAAGGCUAGUCCGU | 931 |
| UAUCAACUUGAAAAAGUGGCACCGAGUCGgugcCAGUUCCUUGGACCAGGCACAUAAUUAAC | |
| CCUAAAGUUAAUUAUGUACCguuuuagaGCUAGAAAUAGCAAGUUAAAAUAAGGCUAGUCCGU | 932 |
| UAUCAACUUGAAAAAGUGGCACCGAGUCGgugcCAGUUCCUUGGACCAGGCACAUAAUUAACU | |
| CCUAAAGUUAAUUAUGUACCguuuuagaGCUAGAAAUAGCAAGUUAAAAUAAGGCUAGUCCGU | 933 |
| UAUCAACUUGAAAAAGUGGCACCGAGUCGgugcCAGUUCCUUGGACCAGGCACAUAAUUAACU | |
| U | |
| CCUAAAGUUAAUUAUGUACCguuuuagaGCUAGAAAUAGCAAGUUAAAAUAAGGCUAGUCCGU | 934 |
| UAUCAACUUGAAAAAGUGGCACCGAGUCGgugcCAGUUCCUUGGACCAGGCACAUAAUUAACU | |
| UU | |
| CCUAAAGUUAAUUAUGUACCguuuuagaGCUAGAAAUAGCAAGUUAAAAUAAGGCUAGUCCGU | 935 |
| UAUCAACUUGAAAAAGUGGCACCGAGUCGgugcCAGUUCCUUGGACCAGGCACAUAAUUAACU | |
| UUA | |
| CCUAAAGUUAAUUAUGUACCguuuuagaGCUAGAAAUAGCAAGUUAAAAUAAGGCUAGUCCGU | 936 |
| UAUCAACUUGAAAAAGUGGCACCGAGUCGgugcCCAGUUCCUUGGACCAGGCACAUAAUUAA | |
| CCUAAAGUUAAUUAUGUACCguuuuagaGCUAGAAAUAGCAAGUUAAAAUAAGGCUAGUCCGU | 937 |
| UAUCAACUUGAAAAAGUGGCACCGAGUCGgugcCCAGUUCCUUGGACCAGGCACAUAAUUAAC | |
| CCUAAAGUUAAUUAUGUACCguuuuagaGCUAGAAAUAGCAAGUUAAAAUAAGGCUAGUCCGU | 938 |
| UAUCAACUUGAAAAAGUGGCACCGAGUCGgugcCCAGUUCCUUGGACCAGGCACAUAAUUAAC | |
| U | |
| CCUAAAGUUAAUUAUGUACCguuuuagaGCUAGAAAUAGCAAGUUAAAAUAAGGCUAGUCCGU | 939 |
| UAUCAACUUGAAAAAGUGGCACCGAGUCGgugcCCAGUUCCUUGGACCAGGCACAUAAUUAAC | |
| UU | |
| CCUAAAGUUAAUUAUGUACCguuuuagaGCUAGAAAUAGCAAGUUAAAAUAAGGCUAGUCCGU | 940 |
| UAUCAACUUGAAAAAGUGGCACCGAGUCGgugcCCAGUUCCUUGGACCAGGCACAUAAUUAAC | |
| UUU | |
| CCUAAAGUUAAUUAUGUACCguuuuagaGCUAGAAAUAGCAAGUUAAAAUAAGGCUAGUCCGU | 941 |
| UAUCAACUUGAAAAAGUGGCACCGAGUCGgugcCCAGUUCCUUGGACCAGGCACAUAAUUAAC | |
| UUUA | |
| CCUAAAGUUAAUUAUGUACCguuuuagaGCUAGAAAUAGCAAGUUAAAAUAAGGCUAGUCCGU | 942 |
| UAUCAACUUGAAAAAGUGGCACCGAGUCGgugcGCCAGUUCCUUGGACCAGGCACAUAAUUAA | |
| CCUAAAGUUAAUUAUGUACCguuuuagaGCUAGAAAUAGCAAGUUAAAAUAAGGCUAGUCCGU | 943 |
| UAUCAACUUGAAAAAGUGGCACCGAGUCGgugcGCCAGUUCCUUGGACCAGGCACAUAAUUAA | |
| C | |
| CCUAAAGUUAAUUAUGUACCguuuuagaGCUAGAAAUAGCAAGUUAAAAUAAGGCUAGUCCGU | 944 |
| UAUCAACUUGAAAAAGUGGCACCGAGUCGgugcGCCAGUUCCUUGGACCAGGCACAUAAUUAA | |
| CU | |
| CCUAAAGUUAAUUAUGUACCguuuuagaGCUAGAAAUAGCAAGUUAAAAUAAGGCUAGUCCGU | 945 |
| UAUCAACUUGAAAAAGUGGCACCGAGUCGgugcGCCAGUUCCUUGGACCAGGCACAUAAUUAA | |
| CUU | |
| CCUAAAGUUAAUUAUGUACCguuuuagaGCUAGAAAUAGCAAGUUAAAAUAAGGCUAGUCCGU | 946 |
| UAUCAACUUGAAAAAGUGGCACCGAGUCGgugcGCCAGUUCCUUGGACCAGGCACAUAAUUAA | |
| CUUU | |
| CCUAAAGUUAAUUAUGUACCguuuuagaGCUAGAAAUAGCAAGUUAAAAUAAGGCUAGUCCGU | 947 |
| UAUCAACUUGAAAAAGUGGCACCGAGUCGgugcGCCAGUUCCUUGGACCAGGCACAUAAUUAA | |
| CUUUA | |
| CCUAAAGUUAAUUAUGUACCguuuuagaGCUAGAAAUAGCAAGUUAAAAUAAGGCUAGUCCGU | 948 |
| UAUCAACUUGAAAAAGUGGCACCGAGUCGgugcUGCCAGUUCCUUGGACCAGGCACAUAAUUA | |
| A | |
| CCUAAAGUUAAUUAUGUACCguuuuagaGCUAGAAAUAGCAAGUUAAAAUAAGGCUAGUCCGU | 949 |
| UAUCAACUUGAAAAAGUGGCACCGAGUCGgugcUGCCAGUUCCUUGGACCAGGCACAUAAUUA | |
| AC | |
| CCUAAAGUUAAUUAUGUACCguuuuagaGCUAGAAAUAGCAAGUUAAAAUAAGGCUAGUCCGU | 950 |
| UAUCAACUUGAAAAAGUGGCACCGAGUCGgugcUGCCAGUUCCUUGGACCAGGCACAUAAUUA | |
| ACU | |
| CCUAAAGUUAAUUAUGUACCguuuuagaGCUAGAAAUAGCAAGUUAAAAUAAGGCUAGUCCGU | 951 |
| UAUCAACUUGAAAAAGUGGCACCGAGUCGgugcUGCCAGUUCCUUGGACCAGGCACAUAAUUA | |
| ACUU | |
| CCUAAAGUUAAUUAUGUACCguuuuagaGCUAGAAAUAGCAAGUUAAAAUAAGGCUAGUCCGU | 952 |
| UAUCAACUUGAAAAAGUGGCACCGAGUCGgugcUGCCAGUUCCUUGGACCAGGCACAUAAUUA | |
| ACUUU | |
| CCUAAAGUUAAUUAUGUACCguuuuagaGCUAGAAAUAGCAAGUUAAAAUAAGGCUAGUCCGU | 953 |
| UAUCAACUUGAAAAAGUGGCACCGAGUCGgugcUGCCAGUUCCUUGGACCAGGCACAUAAUUA | |
| ACUUUA | |
| CCUAAAGUUAAUUAUGUACCguuuuagaGCUAGAAAUAGCAAGUUAAAAUAAGGCUAGUCCGU | 954 |
| UAUCAACUUGAAAAAGUGGCACCGAGUCGgugcAUGCCAGUUCCUUGGACCAGGCACAUAAUU | |
| AA | |
| CCUAAAGUUAAUUAUGUACCguuuuagaGCUAGAAAUAGCAAGUUAAAAUAAGGCUAGUCCGU | 955 |
| UAUCAACUUGAAAAAGUGGCACCGAGUCGgugcAUGCCAGUUCCUUGGACCAGGCACAUAAUU | |
| AAC | |
| CCUAAAGUUAAUUAUGUACCguuuuagaGCUAGAAAUAGCAAGUUAAAAUAAGGCUAGUCCGU | 956 |
| UAUCAACUUGAAAAAGUGGCACCGAGUCGgugcAUGCCAGUUCCUUGGACCAGGCACAUAAUU | |
| AACU | |
| CCUAAAGUUAAUUAUGUACCguuuuagaGCUAGAAAUAGCAAGUUAAAAUAAGGCUAGUCCGU | 957 |
| UAUCAACUUGAAAAAGUGGCACCGAGUCGgugcAUGCCAGUUCCUUGGACCAGGCACAUAAUU | |
| AACUU | |
| CCUAAAGUUAAUUAUGUACCguuuuagaGCUAGAAAUAGCAAGUUAAAAUAAGGCUAGUCCGU | 958 |
| UAUCAACUUGAAAAAGUGGCACCGAGUCGgugcAUGCCAGUUCCUUGGACCAGGCACAUAAUU | |
| AACUUU | |
| CCUAAAGUUAAUUAUGUACCguuuuagaGCUAGAAAUAGCAAGUUAAAAUAAGGCUAGUCCGU | 959 |
| UAUCAACUUGAAAAAGUGGCACCGAGUCGgugcAUGCCAGUUCCUUGGACCAGGCACAUAAUU | |
| AACUUUA | |
| CCUAAAGUUAAUUAUGUACCguuuuagaGCUAGAAAUAGCAAGUUAAAAUAAGGCUAGUCCGU | 960 |
| UAUCAACUUGAAAAAGUGGCACCGAGUCGgugcGAUGCCAGUUCCUUGGACCAGGCACAUAAU | |
| UAA | |
| CCUAAAGUUAAUUAUGUACCguuuuagaGCUAGAAAUAGCAAGUUAAAAUAAGGCUAGUCCGU | 961 |
| UAUCAACUUGAAAAAGUGGCACCGAGUCGgugcGAUGCCAGUUCCUUGGACCAGGCACAUAAU | |
| UAAC | |
| CCUAAAGUUAAUUAUGUACCguuuuagaGCUAGAAAUAGCAAGUUAAAAUAAGGCUAGUCCGU | 962 |
| UAUCAACUUGAAAAAGUGGCACCGAGUCGgugcGAUGCCAGUUCCUUGGACCAGGCACAUAAU | |
| UAACU | |
| CCUAAAGUUAAUUAUGUACCguuuuagaGCUAGAAAUAGCAAGUUAAAAUAAGGCUAGUCCGU | 963 |
| UAUCAACUUGAAAAAGUGGCACCGAGUCGgugcGAUGCCAGUUCCUUGGACCAGGCACAUAAU | |
| UAACUU | |
| CCUAAAGUUAAUUAUGUACCguuuuagaGCUAGAAAUAGCAAGUUAAAAUAAGGCUAGUCCGU | 964 |
| UAUCAACUUGAAAAAGUGGCACCGAGUCGgugcGAUGCCAGUUCCUUGGACCAGGCACAUAAU | |
| UAACUUU | |
| CCUAAAGUUAAUUAUGUACCguuuuagaGCUAGAAAUAGCAAGUUAAAAUAAGGCUAGUCCGU | 965 |
| UAUCAACUUGAAAAAGUGGCACCGAGUCGgugcGAUGCCAGUUCCUUGGACCAGGCACAUAAU | |
| UAACUUUA | |
| CCUAAAGUUAAUUAUGUACCguuuuagaGCUAGAAAUAGCAAGUUAAAAUAAGGCUAGUCCGU | 966 |
| UAUCAACUUGAAAAAGUGGCACCGAGUCGgugcGGAUGCCAGUUCCUUGGACCAGGCACAUAA | |
| UUAA | |
| CCUAAAGUUAAUUAUGUACCguuuuagaGCUAGAAAUAGCAAGUUAAAAUAAGGCUAGUCCGU | 967 |
| UAUCAACUUGAAAAAGUGGCACCGAGUCGgugcGGAUGCCAGUUCCUUGGACCAGGCACAUAA | |
| UUAAC | |
| CCUAAAGUUAAUUAUGUACCguuuuagaGCUAGAAAUAGCAAGUUAAAAUAAGGCUAGUCCGU | 968 |
| UAUCAACUUGAAAAAGUGGCACCGAGUCGgugcGGAUGCCAGUUCCUUGGACCAGGCACAUAA | |
| UUAACU | |
| CCUAAAGUUAAUUAUGUACCguuuuagaGCUAGAAAUAGCAAGUUAAAAUAAGGCUAGUCCGU | 969 |
| UAUCAACUUGAAAAAGUGGCACCGAGUCGgugcGGAUGCCAGUUCCUUGGACCAGGCACAUAA | |
| UUAACUU | |
| CCUAAAGUUAAUUAUGUACCguuuuagaGCUAGAAAUAGCAAGUUAAAAUAAGGCUAGUCCGU | 970 |
| UAUCAACUUGAAAAAGUGGCACCGAGUCGgugcGGAUGCCAGUUCCUUGGACCAGGCACAUAA | |
| UUAACUUU | |
| CCUAAAGUUAAUUAUGUACCguuuuagaGCUAGAAAUAGCAAGUUAAAAUAAGGCUAGUCCGU | 971 |
| UAUCAACUUGAAAAAGUGGCACCGAGUCGgugcGGAUGCCAGUUCCUUGGACCAGGCACAUAA | |
| UUAACUUUA | |
| CCUAAAGUUAAUUAUGUACCguuuuagaGCUAGAAAUAGCAAGUUAAAAUAAGGCUAGUCCGU | 972 |
| UAUCAACUUGAAAAAGUGGCACCGAGUCGgugcGGGAUGCCAGUUCCUUGGACCAGGCACAUA | |
| AUUAA | |
| CCUAAAGUUAAUUAUGUACCguuuuagaGCUAGAAAUAGCAAGUUAAAAUAAGGCUAGUCCGU | 973 |
| UAUCAACUUGAAAAAGUGGCACCGAGUCGgugcGGGAUGCCAGUUCCUUGGACCAGGCACAUA | |
| AUUAAC | |
| CCUAAAGUUAAUUAUGUACCguuuuagaGCUAGAAAUAGCAAGUUAAAAUAAGGCUAGUCCGU | 974 |
| UAUCAACUUGAAAAAGUGGCACCGAGUCGgugcGGGAUGCCAGUUCCUUGGACCAGGCACAUA | |
| AUUAACU | |
| CCUAAAGUUAAUUAUGUACCguuuuagaGCUAGAAAUAGCAAGUUAAAAUAAGGCUAGUCCGU | 975 |
| UAUCAACUUGAAAAAGUGGCACCGAGUCGgugcGGGAUGCCAGUUCCUUGGACCAGGCACAUA | |
| AUUAACUU | |
| CCUAAAGUUAAUUAUGUACCguuuuagaGCUAGAAAUAGCAAGUUAAAAUAAGGCUAGUCCGU | 976 |
| UAUCAACUUGAAAAAGUGGCACCGAGUCGgugcGGGAUGCCAGUUCCUUGGACCAGGCACAUA | |
| AUUAACUUU | |
| CCUAAAGUUAAUUAUGUACCguuuuagaGCUAGAAAUAGCAAGUUAAAAUAAGGCUAGUCCGU | 977 |
| UAUCAACUUGAAAAAGUGGCACCGAGUCGgugcGGGAUGCCAGUUCCUUGGACCAGGCACAUA | |
| AUUAACUUUA | |
| CCUAAAGUUAAUUAUGUACCguuuuagaGCUAGAAAUAGCAAGUUAAAAUAAGGCUAGUCCGU | 978 |
| UAUCAACUUGAAAAAGUGGCACCGAGUCGgugcGGGGAUGCCAGUUCCUUGGACCAGGCACAU | |
| AAUUAA | |
| CCUAAAGUUAAUUAUGUACCguuuuagaGCUAGAAAUAGCAAGUUAAAAUAAGGCUAGUCCGU | 979 |
| UAUCAACUUGAAAAAGUGGCACCGAGUCGgugcGGGGAUGCCAGUUCCUUGGACCAGGCACAU | |
| AAUUAAC | |
| CCUAAAGUUAAUUAUGUACCguuuuagaGCUAGAAAUAGCAAGUUAAAAUAAGGCUAGUCCGU | 980 |
| UAUCAACUUGAAAAAGUGGCACCGAGUCGgugcGGGGAUGCCAGUUCCUUGGACCAGGCACAU | |
| AAUUAACU | |
| CCUAAAGUUAAUUAUGUACCguuuuagaGCUAGAAAUAGCAAGUUAAAAUAAGGCUAGUCCGU | 981 |
| UAUCAACUUGAAAAAGUGGCACCGAGUCGgugcGGGGAUGCCAGUUCCUUGGACCAGGCACAU | |
| AAUUAACUU | |
| CCUAAAGUUAAUUAUGUACCguuuuagaGCUAGAAAUAGCAAGUUAAAAUAAGGCUAGUCCGU | 982 |
| UAUCAACUUGAAAAAGUGGCACCGAGUCGgugcGGGGAUGCCAGUUCCUUGGACCAGGCACAU | |
| AAUUAACUUU | |
| CCUAAAGUUAAUUAUGUACCguuuuagaGCUAGAAAUAGCAAGUUAAAAUAAGGCUAGUCCGU | 983 |
| UAUCAACUUGAAAAAGUGGCACCGAGUCGgugcGGGGAUGCCAGUUCCUUGGACCAGGCACAU | |
| AAUUAACUUUA | |
| CCUAAAGUUAAUUAUGUACCguuuuagaGCUAGAAAUAGCAAGUUAAAAUAAGGCUAGUCCGU | 984 |
| UAUCAACUUGAAAAAGUGGCACCGAGUCGgugcAGGGGAUGCCAGUUCCUUGGACCAGGCACA | |
| UAAUUAA | |
| CCUAAAGUUAAUUAUGUACCguuuuagaGCUAGAAAUAGCAAGUUAAAAUAAGGCUAGUCCGU | 985 |
| UAUCAACUUGAAAAAGUGGCACCGAGUCGgugcAGGGGAUGCCAGUUCCUUGGACCAGGCACA | |
| UAAUUAAC | |
| CCUAAAGUUAAUUAUGUACCguuuuagaGCUAGAAAUAGCAAGUUAAAAUAAGGCUAGUCCGU | 986 |
| UAUCAACUUGAAAAAGUGGCACCGAGUCGgugcAGGGGAUGCCAGUUCCUUGGACCAGGCACA | |
| UAAUUAACU | |
| CCUAAAGUUAAUUAUGUACCguuuuagaGCUAGAAAUAGCAAGUUAAAAUAAGGCUAGUCCGU | 987 |
| UAUCAACUUGAAAAAGUGGCACCGAGUCGgugcAGGGGAUGCCAGUUCCUUGGACCAGGCACA | |
| UAAUUAACUU | |
| CCUAAAGUUAAUUAUGUACCguuuuagaGCUAGAAAUAGCAAGUUAAAAUAAGGCUAGUCCGU | 988 |
| UAUCAACUUGAAAAAGUGGCACCGAGUCGgugcAGGGGAUGCCAGUUCCUUGGACCAGGCACA | |
| UAAUUAACUUU | |
| CCUAAAGUUAAUUAUGUACCguuuuagaGCUAGAAAUAGCAAGUUAAAAUAAGGCUAGUCCGU | 989 |
| UAUCAACUUGAAAAAGUGGCACCGAGUCGgugcAGGGGAUGCCAGUUCCUUGGACCAGGCACA | |
| UAAUUAACUUUA | |
| CCUAAAGUUAAUUAUGUACCguuuuagaGCUAGAAAUAGCAAGUUAAAAUAAGGCUAGUCCGU | 990 |
| UAUCAACUUGAAAAAGUGGCACCGAGUCGgugcUAGGGGAUGCCAGUUCCUUGGACCAGGCAC | |
| AUAAUUAA | |
| CCUAAAGUUAAUUAUGUACCguuuuagaGCUAGAAAUAGCAAGUUAAAAUAAGGCUAGUCCGU | 991 |
| UAUCAACUUGAAAAAGUGGCACCGAGUCGgugcUAGGGGAUGCCAGUUCCUUGGACCAGGCAC | |
| AUAAUUAAC | |
| CCUAAAGUUAAUUAUGUACCguuuuagaGCUAGAAAUAGCAAGUUAAAAUAAGGCUAGUCCGU | 992 |
| UAUCAACUUGAAAAAGUGGCACCGAGUCGgugcUAGGGGAUGCCAGUUCCUUGGACCAGGCAC | |
| AUAAUUAACU | |
| CCUAAAGUUAAUUAUGUACCguuuuagaGCUAGAAAUAGCAAGUUAAAAUAAGGCUAGUCCGU | 993 |
| UAUCAACUUGAAAAAGUGGCACCGAGUCGgugcUAGGGGAUGCCAGUUCCUUGGACCAGGCAC | |
| AUAAUUAACUU | |
| CCUAAAGUUAAUUAUGUACCguuuuagaGCUAGAAAUAGCAAGUUAAAAUAAGGCUAGUCCGU | 994 |
| UAUCAACUUGAAAAAGUGGCACCGAGUCGgugcUAGGGGAUGCCAGUUCCUUGGACCAGGCAC | |
| AUAAUUAACUUU | |
| CCUAAAGUUAAUUAUGUACCguuuuagaGCUAGAAAUAGCAAGUUAAAAUAAGGCUAGUCCGU | 995 |
| UAUCAACUUGAAAAAGUGGCACCGAGUCGgugcUAGGGGAUGCCAGUUCCUUGGACCAGGCAC | |
| AUAAUUAACUUUA | |
| CCUAAAGUUAAUUAUGUACCguuuuagaGCUAGAAAUAGCAAGUUAAAAUAAGGCUAGUCCGU | 996 |
| UAUCAACUUGAAAAAGUGGCACCGAGUCGgugcCUAGGGGAUGCCAGUUCCUUGGACCAGGCA | |
| CAUAAUUAA | |
| CCUAAAGUUAAUUAUGUACCguuuuagaGCUAGAAAUAGCAAGUUAAAAUAAGGCUAGUCCGU | 997 |
| UAUCAACUUGAAAAAGUGGCACCGAGUCGgugcCUAGGGGAUGCCAGUUCCUUGGACCAGGCA | |
| CAUAAUUAAC | |
| CCUAAAGUUAAUUAUGUACCguuuuagaGCUAGAAAUAGCAAGUUAAAAUAAGGCUAGUCCGU | 998 |
| UAUCAACUUGAAAAAGUGGCACCGAGUCGgugcCUAGGGGAUGCCAGUUCCUUGGACCAGGCA | |
| CAUAAUUAACU | |
| CCUAAAGUUAAUUAUGUACCguuuuagaGCUAGAAAUAGCAAGUUAAAAUAAGGCUAGUCCGU | 999 |
| UAUCAACUUGAAAAAGUGGCACCGAGUCGgugcCUAGGGGAUGCCAGUUCCUUGGACCAGGCA | |
| CAUAAUUAACUU | |
| CCUAAAGUUAAUUAUGUACCguuuuagaGCUAGAAAUAGCAAGUUAAAAUAAGGCUAGUCCGU | 1000 |
| UAUCAACUUGAAAAAGUGGCACCGAGUCGgugcCUAGGGGAUGCCAGUUCCUUGGACCAGGcA | |
| CAUAAUUAACUUU | |
| CCUAAAGUUAAUUAUGUACCguuuuagaGCUAGAAAUAGCAAGUUAAAAUAAGGCUAGUCCGU | 1001 |
| UAUCAACUUGAAAAAGUGGCACCGAGUCGgugcCUAGGGGAUGCCAGUUCCUUGGACCAGGCA | |
| CAUAAUUAACUUUA | |
| CCUAAAGUUAAUUAUGUACCguuuuagaGCUAGAAAUAGCAAGUUAAAAUAAGGCUAGUCCGU | 1002 |
| UAUCAACUUGAAAAAGUGGCACCGAGUCGgugcGCUAGGGGAUGCCAGUUCCUUGGACCAGGC | |
| ACAUAAUUAA | |
| CCUAAAGUUAAUUAUGUACCguuuuagaGCUAGAAAUAGCAAGUUAAAAUAAGGCUAGUCCGU | 1003 |
| UAUCAACUUGAAAAAGUGGCACCGAGUCGgugcGCUAGGGGAUGCCAGUUCCUUGGACCAGGc | |
| ACAUAAUUAAC | |
| CCUAAAGUUAAUUAUGUACCguuuuagaGCUAGAAAUAGCAAGUUAAAAUAAGGCUAGUCCGU | 1004 |
| UAUCAACUUGAAAAAGUGGCACCGAGUCGgugcGCUAGGGGAUGCCAGUUCCUUGGACCAGG | |
| ACAUAAUUAACU | |
| CCUAAAGUUAAUUAUGUACCguuuuagaGCUAGAAAUAGCAAGUUAAAAUAAGGCUAGUCCGU | 1005 |
| UAUCAACUUGAAAAAGUGGCACCGAGUCGgugcGCUAGGGGAUGCCAGUUCCUUGGACCAGGc | |
| ACAUAAUUAACUU | |
| CCUAAAGUUAAUUAUGUACCguuuuagaGCUAGAAAUAGCAAGUUAAAAUAAGGCUAGUCCGU | 1006 |
| UAUCAACUUGAAAAAGUGGCACCGAGUCGgugcGCUAGGGGAUGCCAGUUCCUUGGACCAGGC | |
| ACAUAAUUAACUUU | |
| CCUAAAGUUAAUUAUGUACCguuuuagaGCUAGAAAUAGCAAGUUAAAAUAAGGCUAGUCCGU | 1007 |
| UAUCAACUUGAAAAAGUGGCACCGAGUCGgugcGCUAGGGGAUGCCAGUUCCUUGGACCAGGC | |
| ACAUAAUUAACUUUA | |
| GGAUGCCAGUUCCUUGGACCguuuuagaGCUAGAAAUAGCAAGUUAAAAUAAGGCUAGUCCGU | 1008 |
| UAUCAACUUGAAAAAGUGGCACCGAGUCGgugcUGUgCCUGGUCCAAGGAACU | |
| GGAUGCCAGUUCCUUGGACCguuuuagaGCUAGAAAUAGCAAGUUAAAAUAAGGCUAGUCCGU | 1009 |
| UAUCAACUUGAAAAAGUGGCACCGAGUCGgugcUGUgCCUGGUCCAAGGAACUG | |
| GGAUGCCAGUUCCUUGGACCguuuuagaGCUAGAAAUAGCAAGUUAAAAUAAGGCUAGUCCGU | 1010 |
| UAUCAACUUGAAAAAGUGGCACCGAGUCGgugcUGUgCCUGGUCCAAGGAACUGG | |
| GGAUGCCAGUUCCUUGGACCguuuuagaGCUAGAAAUAGCAAGUUAAAAUAAGGCUAGUCCGU | 1011 |
| UAUCAACUUGAAAAAGUGGCACCGAGUCGgugcUGUgCCUGGUCCAAGGAACUGGC | |
| GGAUGCCAGUUCCUUGGACCguuuuagaGCUAGAAAUAGCAAGUUAAAAUAAGGCUAGUCCGU | 1012 |
| UAUCAACUUGAAAAAGUGGCACCGAGUCGgugcUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1013 |
| UAUCAACUUGAAAAAGUGGCACCGAGUCGgugcUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1014 |
| UAUCAACUUGAAAAAGUGGCACCGAGUCGgugcAUGUgCCUGGUCCAAGGAACU | |
| GGAUGCCAGUUCCUUGGACCguuuuagaGCUAGAAAUAGCAAGUUAAAAUAAGGCUAGUCCGU | 1015 |
| UAUCAACUUGAAAAAGUGGCACCGAGUCGgugcAUGUgCCUGGUCCAAGGAACUG | |
| GGAUGCCAGUUCCUUGGACCguuuuagaGCUAGAAAUAGCAAGUUAAAAUAAGGCUAGUCCGU | 1016 |
| UAUCAACUUGAAAAAGUGGCACCGAGUCGgugcAUGUgCCUGGUCCAAGGAACUGG | |
| GGAUGCCAGUUCCUUGGACCguuuuagaGCUAGAAAUAGCAAGUUAAAAUAAGGCUAGUCCGU | 1017 |
| UAUCAACUUGAAAAAGUGGCACCGAGUCGgugcAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1018 |
| UAUCAACUUGAAAAAGUGGCACCGAGUCGgugcAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1019 |
| UAUCAACUUGAAAAAGUGGCACCGAGUCGgugcAUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1020 |
| UAUCAACUUGAAAAAGUGGCACCGAGUCGgugcUAUGUgCCUGGUCCAAGGAACU | |
| GGAUGCCAGUUCCUUGGACCguuuuagaGCUAGAAAUAGCAAGUUAAAAUAAGGCUAGUCCGU | 1021 |
| UAUCAACUUGAAAAAGUGGCACCGAGUCGgugcUAUGUgCCUGGUCCAAGGAACUG | |
| GGAUGCCAGUUCCUUGGACCguuuuagaGCUAGAAAUAGCAAGUUAAAAUAAGGCUAGUCCGU | 1022 |
| UAUCAACUUGAAAAAGUGGCACCGAGUCGgugcUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1023 |
| UAUCAACUUGAAAAAGUGGCACCGAGUCGgugcUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1024 |
| UAUCAACUUGAAAAAGUGGCACCGAGUCGgugcUAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1025 |
| UAUCAACUUGAAAAAGUGGCACCGAGUCGgugcUAUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1026 |
| UAUCAACUUGAAAAAGUGGCACCGAGUCGgugcUUAUGUgCCUGGUCCAAGGAACU | |
| GGAUGCCAGUUCCUUGGACCguuuuagaGCUAGAAAUAGCAAGUUAAAAUAAGGCUAGUCCGU | 1027 |
| UAUCAACUUGAAAAAGUGGCACCGAGUCGgugcUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1028 |
| UAUCAACUUGAAAAAGUGGCACCGAGUCGgugcUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1029 |
| UAUCAACUUGAAAAAGUGGCACCGAGUCGgugcUUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1030 |
| UAUCAACUUGAAAAAGUGGCACCGAGUCGgugcUUAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1031 |
| UAUCAACUUGAAAAAGUGGCACCGAGUCGgugcUUAUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1032 |
| UAUCAACUUGAAAAAGUGGCACCGAGUCGgugcAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1033 |
| UAUCAACUUGAAAAAGUGGCACCGAGUCGgugcAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1034 |
| UAUCAACUUGAAAAAGUGGCACCGAGUCGgugcAUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1035 |
| UAUCAACUUGAAAAAGUGGCACCGAGUCGgugcAUUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1036 |
| UAUCAACUUGAAAAAGUGGCACCGAGUCGgugcAUUAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1037 |
| UAUCAACUUGAAAAAGUGGCACCGAGUCGgugcAUUAUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1038 |
| UAUCAACUUGAAAAAGUGGCACCGAGUCGgugcAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1039 |
| UAUCAACUUGAAAAAGUGGCACCGAGUCGgugcAAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1040 |
| UAUCAACUUGAAAAAGUGGCACCGAGUCGgugcAAUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1041 |
| UAUCAACUUGAAAAAGUGGCACCGAGUCGgugcAAUUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1042 |
| UAUCAACUUGAAAAAGUGGCACCGAGUCGgugcAAUUAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1043 |
| UAUCAACUUGAAAAAGUGGCACCGAGUCGgugcAAUUAUGUgCCUGGUCCAAGGAACUGGCAU | |
| GGAUGCCAGUUCCUUGGACCguuuuagaGCUAGAAAUAGCAAGUUAAAAUAAGGCUAGUCCGU | 1044 |
| UAUCAACUUGAAAAAGUGGCACCGAGUCGgugcUAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1045 |
| UAUCAACUUGAAAAAGUGGCACCGAGUCGgugcUAAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1046 |
| UAUCAACUUGAAAAAGUGGCACCGAGUCGgugcUAAUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1047 |
| UAUCAACUUGAAAAAGUGGCACCGAGUCGgugcUAAUUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1048 |
| UAUCAACUUGAAAAAGUGGCACCGAGUCGgugcUAAUUAUGUgCCUGGUCCAAGGAACUGGCA | |
| GGAUGCCAGUUCCUUGGACCguuuuagaGCUAGAAAUAGCAAGUUAAAAUAAGGCUAGUCCGU | 1049 |
| UAUCAACUUGAAAAAGUGGCACCGAGUCGgugcUAAUUAUGUgCCUGGUCCAAGGAACUGGCA | |
| U | |
| GGAUGCCAGUUCCUUGGACCguuuuagaGCUAGAAAUAGCAAGUUAAAAUAAGGCUAGUCCGU | 1050 |
| UAUCAACUUGAAAAAGUGGCACCGAGUCGgugcUUAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1051 |
| UAUCAACUUGAAAAAGUGGCACCGAGUCGgugcUUAAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1052 |
| UAUCAACUUGAAAAAGUGGCACCGAGUCGgugcUUAAUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1053 |
| UAUCAACUUGAAAAAGUGGCACCGAGUCGgugcUUAAUUAUGUgCCUGGUCCAAGGAACUGGC | |
| GGAUGCCAGUUCCUUGGACCguuuuagaGCUAGAAAUAGCAAGUUAAAAUAAGGCUAGUCCGU | 1054 |
| UAUCAACUUGAAAAAGUGGCACCGAGUCGgugcUUAAUUAUGUgCCUGGUCCAAGGAACUGGC | |
| A | |
| GGAUGCCAGUUCCUUGGACCguuuuagaGCUAGAAAUAGCAAGUUAAAAUAAGGCUAGUCCGU | 1055 |
| UAUCAACUUGAAAAAGUGGCACCGAGUCGgugcUUAAUUAUGUgCCUGGUCCAAGGAACUGGC | |
| AU | |
| GGAUGCCAGUUCCUUGGACCguuuuagaGCUAGAAAUAGCAAGUUAAAAUAAGGCUAGUCCGU | 1056 |
| UAUCAACUUGAAAAAGUGGCACCGAGUCGgugcGUUAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1057 |
| UAUCAACUUGAAAAAGUGGCACCGAGUCGgugcGUUAAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1058 |
| UAUCAACUUGAAAAAGUGGCACCGAGUCGgugcGUUAAUUAUGUgCCUGGUCCAAGGAACUGG | |
| GGAUGCCAGUUCCUUGGACCguuuuagaGCUAGAAAUAGCAAGUUAAAAUAAGGCUAGUCCGU | 1059 |
| UAUCAACUUGAAAAAGUGGCACCGAGUCGgugcGUUAAUUAUGUgCCUGGUCCAAGGAACUGG | |
| C | |
| GGAUGCCAGUUCCUUGGACCguuuuagaGCUAGAAAUAGCAAGUUAAAAUAAGGCUAGUCCGU | 1060 |
| UAUCAACUUGAAAAAGUGGCACCGAGUCGgugcGUUAAUUAUGUgCCUGGUCCAAGGAACUGG | |
| CA | |
| GGAUGCCAGUUCCUUGGACCguuuuagaGCUAGAAAUAGCAAGUUAAAAUAAGGCUAGUCCGU | 1061 |
| UAUCAACUUGAAAAAGUGGCACCGAGUCGgugcGUUAAUUAUGUgCCUGGUCCAAGGAACUGG | |
| CAU | |
| GGAUGCCAGUUCCUUGGACCguuuuagaGCUAGAAAUAGCAAGUUAAAAUAAGGCUAGUCCGU | 1062 |
| UAUCAACUUGAAAAAGUGGCACCGAGUCGgugcAGUUAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1063 |
| UAUCAACUUGAAAAAGUGGCACCGAGUCGgugcAGUUAAUUAUGUgCCUGGUCCAAGGAACUG | |
| GGAUGCCAGUUCCUUGGACCguuuuagaGCUAGAAAUAGCAAGUUAAAAUAAGGCUAGUCCGU | 1064 |
| UAUCAACUUGAAAAAGUGGCACCGAGUCGgugcAGUUAAUUAUGUgCCUGGUCCAAGGAACUG | |
| G | |
| GGAUGCCAGUUCCUUGGACCguuuuagaGCUAGAAAUAGCAAGUUAAAAUAAGGCUAGUCCGU | 1065 |
| UAUCAACUUGAAAAAGUGGCACCGAGUCGgugcAGUUAAUUAUGUgCCUGGUCCAAGGAACUG | |
| GC | |
| GGAUGCCAGUUCCUUGGACCguuuuagaGCUAGAAAUAGCAAGUUAAAAUAAGGCUAGUCCGU | 1066 |
| UAUCAACUUGAAAAAGUGGCACCGAGUCGgugcAGUUAAUUAUGUgCCUGGUCCAAGGAACUG | |
| GCA | |
| GGAUGCCAGUUCCUUGGACCguuuuagaGCUAGAAAUAGCAAGUUAAAAUAAGGCUAGUCCGU | 1067 |
| UAUCAACUUGAAAAAGUGGCACCGAGUCGgugcAGUUAAUUAUGUgCCUGGUCCAAGGAACUG | |
| GCAU | |
| GGAUGCCAGUUCCUUGGACCguuuuagaGCUAGAAAUAGCAAGUUAAAAUAAGGCUAGUCCGU | 1068 |
| UAUCAACUUGAAAAAGUGGCACCGAGUCGgugcAAGUUAAUUAUGUgCCUGGUCCAAGGAACU | |
| GGAUGCCAGUUCCUUGGACCguuuuagaGCUAGAAAUAGCAAGUUAAAAUAAGGCUAGUCCGU | 1069 |
| UAUCAACUUGAAAAAGUGGCACCGAGUCGgugcAAGUUAAUUAUGUgCCUGGUCCAAGGAACU | |
| G | |
| GGAUGCCAGUUCCUUGGACCguuuuagaGCUAGAAAUAGCAAGUUAAAAUAAGGCUAGUCCGU | 1070 |
| UAUCAACUUGAAAAAGUGGCACCGAGUCGgugcAAGUUAAUUAUGUgCCUGGUCCAAGGAACU | |
| GG | |
| GGAUGCCAGUUCCUUGGACCguuuuagaGCUAGAAAUAGCAAGUUAAAAUAAGGCUAGUCCGU | 1071 |
| UAUCAACUUGAAAAAGUGGCACCGAGUCGgugcAAGUUAAUUAUGUgCCUGGUCCAAGGAACU | |
| GGC | |
| GGAUGCCAGUUCCUUGGACCguuuuagaGCUAGAAAUAGCAAGUUAAAAUAAGGCUAGUCCGU | 1072 |
| UAUCAACUUGAAAAAGUGGCACCGAGUCGgugcAAGUUAAUUAUGUgCCUGGUCCAAGGAACU | |
| GGCA | |
| GGAUGCCAGUUCCUUGGACCguuuuagaGCUAGAAAUAGCAAGUUAAAAUAAGGCUAGUCCGU | 1073 |
| UAUCAACUUGAAAAAGUGGCACCGAGUCGgugcAAGUUAAUUAUGUgCCUGGUCCAAGGAACU | |
| GGCAU | |
| GGAUGCCAGUUCCUUGGACCguuuuagaGCUAGAAAUAGCAAGUUAAAAUAAGGCUAGUCCGU | 1074 |
| UAUCAACUUGAAAAAGUGGCACCGAGUCGgugcAAAGUUAAUUAUGUgCCUGGUCCAAGGAAC | |
| U | |
| GGAUGCCAGUUCCUUGGACCguuuuagaGCUAGAAAUAGCAAGUUAAAAUAAGGCUAGUCCGU | 1075 |
| UAUCAACUUGAAAAAGUGGCACCGAGUCGgugcAAAGUUAAUUAUGUgCCUGGUCCAAGGAAC | |
| UG | |
| GGAUGCCAGUUCCUUGGACCguuuuagaGCUAGAAAUAGCAAGUUAAAAUAAGGCUAGUCCGU | 1076 |
| UAUCAACUUGAAAAAGUGGCACCGAGUCGgugcAAAGUUAAUUAUGUgCCUGGUCCAAGGAAC | |
| UGG | |
| GGAUGCCAGUUCCUUGGACCguuuuagaGCUAGAAAUAGCAAGUUAAAAUAAGGCUAGUCCGU | 1077 |
| UAUCAACUUGAAAAAGUGGCACCGAGUCGgugcAAAGUUAAUUAUGUgCCUGGUCCAAGGAAC | |
| UGGC | |
| GGAUGCCAGUUCCUUGGACCguuuuagaGCUAGAAAUAGCAAGUUAAAAUAAGGCUAGUCCGU | 1078 |
| UAUCAACUUGAAAAAGUGGCACCGAGUCGgugcAAAGUUAAUUAUGUgCCUGGUCCAAGGAAC | |
| UGGCA | |
| GGAUGCCAGUUCCUUGGACCguuuuagaGCUAGAAAUAGCAAGUUAAAAUAAGGCUAGUCCGU | 1079 |
| UAUCAACUUGAAAAAGUGGCACCGAGUCGgugcAAAGUUAAUUAUGUgCCUGGUCCAAGGAAC | |
| UGGCAU | |
| GGAUGCCAGUUCCUUGGACCguuuuagaGCUAGAAAUAGCAAGUUAAAAUAAGGCUAGUCCGU | 1080 |
| UAUCAACUUGAAAAAGUGGCACCGAGUCGgugcUAAAGUUAAUUAUGUgCCUGGUCCAAGGAA | |
| CU | |
| GGAUGCCAGUUCCUUGGACCguuuuagaGCUAGAAAUAGCAAGUUAAAAUAAGGCUAGUCCGU | 1081 |
| UAUCAACUUGAAAAAGUGGCACCGAGUCGgugcUAAAGUUAAUUAUGUgCCUGGUCCAAGGAA | |
| CUG | |
| GGAUGCCAGUUCCUUGGACCguuuuagaGCUAGAAAUAGCAAGUUAAAAUAAGGCUAGUCCGU | 1082 |
| UAUCAACUUGAAAAAGUGGCACCGAGUCGgugcUAAAGUUAAUUAUGUgCCUGGUCCAAGGAA | |
| CUGG | |
| GGAUGCCAGUUCCUUGGACCguuuuagaGCUAGAAAUAGCAAGUUAAAAUAAGGCUAGUCCGU | 1083 |
| UAUCAACUUGAAAAAGUGGCACCGAGUCGgugcUAAAGUUAAUUAUGUgCCUGGUCCAAGGAA | |
| CUGGC | |
| GGAUGCCAGUUCCUUGGACCguuuuagaGCUAGAAAUAGCAAGUUAAAAUAAGGCUAGUCCGU | 1084 |
| UAUCAACUUGAAAAAGUGGCACCGAGUCGgugcUAAAGUUAAUUAUGUgCCUGGUCCAAGGAA | |
| CUGGCA | |
| GGAUGCCAGUUCCUUGGACCguuuuagaGCUAGAAAUAGCAAGUUAAAAUAAGGCUAGUCCGU | 1085 |
| UAUCAACUUGAAAAAGUGGCACCGAGUCGgugcUAAAGUUAAUUAUGUgCCUGGUCCAAGGAA | |
| CUGGCAU | |
| GGAUGCCAGUUCCUUGGACCguuuuagaGCUAGAAAUAGCAAGUUAAAAUAAGGCUAGUCCGU | 1086 |
| UAUCAACUUGAAAAAGUGGCACCGAGUCGgugcCUAAAGUUAAUUAUGUgCCUGGUCCAAGGA | |
| ACU | |
| GGAUGCCAGUUCCUUGGACCguuuuagaGCUAGAAAUAGCAAGUUAAAAUAAGGCUAGUCCGU | 1087 |
| UAUCAACUUGAAAAAGUGGCACCGAGUCGgugcCUAAAGUUAAUUAUGUgCCUGGUCCAAGGA | |
| ACUG | |
| GGAUGCCAGUUCCUUGGACCguuuuagaGCUAGAAAUAGCAAGUUAAAAUAAGGCUAGUCCGU | 1088 |
| UAUCAACUUGAAAAAGUGGCACCGAGUCGgugcCUAAAGUUAAUUAUGUgCCUGGUCCAAGGA | |
| ACUGG | |
| GGAUGCCAGUUCCUUGGACCguuuuagaGCUAGAAAUAGCAAGUUAAAAUAAGGCUAGUCCGU | 1089 |
| UAUCAACUUGAAAAAGUGGCACCGAGUCGgugcCUAAAGUUAAUUAUGUgCCUGGUCCAAGGA | |
| ACUGGC | |
| GGAUGCCAGUUCCUUGGACCguuuuagaGCUAGAAAUAGCAAGUUAAAAUAAGGCUAGUCCGU | 1090 |
| UAUCAACUUGAAAAAGUGGCACCGAGUCGgugcCUAAAGUUAAUUAUGUgCCUGGUCCAAGGA | |
| ACUGGCA | |
| GGAUGCCAGUUCCUUGGACCguuuuagaGCUAGAAAUAGCAAGUUAAAAUAAGGCUAGUCCGU | 1091 |
| UAUCAACUUGAAAAAGUGGCACCGAGUCGgugcCUAAAGUUAAUUAUGUgCCUGGUCCAAGGA | |
| ACUGGCAU | |
| GGAUGCCAGUUCCUUGGACCguuuuagaGCUAGAAAUAGCAAGUUAAAAUAAGGCUAGUCCGU | 1092 |
| UAUCAACUUGAAAAAGUGGCACCGAGUCGgugcCCUAAAGUUAAUUAUGUgCCUGGUCCAAGG | |
| AACU | |
| GGAUGCCAGUUCCUUGGACCguuuuagaGCUAGAAAUAGCAAGUUAAAAUAAGGCUAGUCCGU | 1093 |
| UAUCAACUUGAAAAAGUGGCACCGAGUCGgugcCCUAAAGUUAAUUAUGUgCCUGGUCCAAGG | |
| AACUG | |
| GGAUGCCAGUUCCUUGGACCguuuuagaGCUAGAAAUAGCAAGUUAAAAUAAGGCUAGUCCGU | 1094 |
| UAUCAACUUGAAAAAGUGGCACCGAGUCGgugcCCUAAAGUUAAUUAUGUgCCUGGUCCAAGG | |
| AACUGG | |
| GGAUGCCAGUUCCUUGGACCguuuuagaGCUAGAAAUAGCAAGUUAAAAUAAGGCUAGUCCGU | 1095 |
| UAUCAACUUGAAAAAGUGGCACCGAGUCGgugcCCUAAAGUUAAUUAUGUgCCUGGUCCAAGG | |
| AACUGGC | |
| GGAUGCCAGUUCCUUGGACCguuuuagaGCUAGAAAUAGCAAGUUAAAAUAAGGCUAGUCCGU | 1096 |
| UAUCAACUUGAAAAAGUGGCACCGAGUCGgugcCCUAAAGUUAAUUAUGUgCCUGGUCCAAGG | |
| AACUGGCA | |
| GGAUGCCAGUUCCUUGGACCguuuuagaGCUAGAAAUAGCAAGUUAAAAUAAGGCUAGUCCGU | 1097 |
| UAUCAACUUGAAAAAGUGGCACCGAGUCGgugcCCUAAAGUUAAUUAUGUgCCUGGUCCAAGG | |
| AACUGGCAU | |
| GGAUGCCAGUUCCUUGGACCguuuuagaGCUAGAAAUAGCAAGUUAAAAUAAGGCUAGUCCGU | 1098 |
| UAUCAACUUGAAAAAGUGGCACCGAGUCGgugcACCUAAAGUUAAUUAUGUgCCUGGUCCAAG | |
| GAACU | |
| GGAUGCCAGUUCCUUGGACCguuuuagaGCUAGAAAUAGCAAGUUAAAAUAAGGCUAGUCCGU | 1099 |
| UAUCAACUUGAAAAAGUGGCACCGAGUCGgugcACCUAAAGUUAAUUAUGUgCCUGGUCCAAG | |
| GAACUG | |
| GGAUGCCAGUUCCUUGGACCguuuuagaGCUAGAAAUAGCAAGUUAAAAUAAGGCUAGUCCGU | 1100 |
| UAUCAACUUGAAAAAGUGGCACCGAGUCGgugcACCUAAAGUUAAUUAUGUgCCUGGUCCAAG | |
| GAACUGG | |
| GGAUGCCAGUUCCUUGGACCguuuuagaGCUAGAAAUAGCAAGUUAAAAUAAGGCUAGUCCGU | 1101 |
| UAUCAACUUGAAAAAGUGGCACCGAGUCGgugcACCUAAAGUUAAUUAUGUgCCUGGUCCAAG | |
| GAACUGGC | |
| GGAUGCCAGUUCCUUGGACCguuuuagaGCUAGAAAUAGCAAGUUAAAAUAAGGCUAGUCCGU | 1102 |
| UAUCAACUUGAAAAAGUGGCACCGAGUCGgugcACCUAAAGUUAAUUAUGUgCCUGGUCCAAG | |
| GAACUGGCA | |
| GGAUGCCAGUUCCUUGGACCguuuuagaGCUAGAAAUAGCAAGUUAAAAUAAGGCUAGUCCGU | 1103 |
| UAUCAACUUGAAAAAGUGGCACCGAGUCGgugcACCUAAAGUUAAUUAUGUgCCUGGUCCAAG | |
| GAACUGGCAU | |
| GGAUGCCAGUUCCUUGGACCguuuuagaGCUAGAAAUAGCAAGUUAAAAUAAGGCUAGUCCGU | 1104 |
| UAUCAACUUGAAAAAGUGGCACCGAGUCGgugcUACCUAAAGUUAAUUAUGUgCCUGGUCCAA | |
| GGAACU | |
| GGAUGCCAGUUCCUUGGACCguuuuagaGCUAGAAAUAGCAAGUUAAAAUAAGGCUAGUCCGU | 1105 |
| UAUCAACUUGAAAAAGUGGCACCGAGUCGgugcUACCUAAAGUUAAUUAUGUgCCUGGUCCAA | |
| GGAACUG | |
| GGAUGCCAGUUCCUUGGACCguuuuagaGCUAGAAAUAGCAAGUUAAAAUAAGGCUAGUCCGU | 1106 |
| UAUCAACUUGAAAAAGUGGCACCGAGUCGgugcUACCUAAAGUUAAUUAUGUgCCUGGUCCAA | |
| GGAACUGG | |
| GGAUGCCAGUUCCUUGGACCguuuuagaGCUAGAAAUAGCAAGUUAAAAUAAGGCUAGUCCGU | 1107 |
| UAUCAACUUGAAAAAGUGGCACCGAGUCGgugcUACCUAAAGUUAAUUAUGUgCCUGGUCCAA | |
| GGAACUGGC | |
| GGAUGCCAGUUCCUUGGACCguuuuagaGCUAGAAAUAGCAAGUUAAAAUAAGGCUAGUCCGU | 1108 |
| UAUCAACUUGAAAAAGUGGCACCGAGUCGgugcUACCUAAAGUUAAUUAUGUgCCUGGUCCAA | |
| GGAACUGGCA | |
| GGAUGCCAGUUCCUUGGACCguuuuagaGCUAGAAAUAGCAAGUUAAAAUAAGGCUAGUCCGU | 1109 |
| UAUCAACUUGAAAAAGUGGCACCGAGUCGgugcUACCUAAAGUUAAUUAUGUgCCUGGUCCAA | |
| GGAACUGGCAU | |
| GGAUGCCAGUUCCUUGGACCguuuuagaGCUAGAAAUAGCAAGUUAAAAUAAGGCUAGUCCGU | 1110 |
| UAUCAACUUGAAAAAGUGGCACCGAGUCGgugcAUACCUAAAGUUAAUUAUGUgCCUGGUCCA | |
| AGGAACU | |
| GGAUGCCAGUUCCUUGGACCguuuuagaGCUAGAAAUAGCAAGUUAAAAUAAGGCUAGUCCGU | 1111 |
| UAUCAACUUGAAAAAGUGGCACCGAGUCGgugcAUACCUAAAGUUAAUUAUGUgCCUGGUCCA | |
| AGGAACUG | |
| GGAUGCCAGUUCCUUGGACCguuuuagaGCUAGAAAUAGCAAGUUAAAAUAAGGCUAGUCCGU | 1112 |
| UAUCAACUUGAAAAAGUGGCACCGAGUCGgugcAUACCUAAAGUUAAUUAUGUgCCUGGUCCA | |
| AGGAACUGG | |
| GGAUGCCAGUUCCUUGGACCguuuuagaGCUAGAAAUAGCAAGUUAAAAUAAGGCUAGUCCGU | 1113 |
| UAUCAACUUGAAAAAGUGGCACCGAGUCGgugcAUACCUAAAGUUAAUUAUGUgCCUGGUCCA | |
| AGGAACUGGC | |
| GGAUGCCAGUUCCUUGGACCguuuuagaGCUAGAAAUAGCAAGUUAAAAUAAGGCUAGUCCGU | 1114 |
| UAUCAACUUGAAAAAGUGGCACCGAGUCGgugcAUACCUAAAGUUAAUUAUGUgCCUGGUCCA | |
| AGGAACUGGCA | |
| GGAUGCCAGUUCCUUGGACCguuuuagaGCUAGAAAUAGCAAGUUAAAAUAAGGCUAGUCCGU | 1115 |
| UAUCAACUUGAAAAAGUGGCACCGAGUCGgugcAUACCUAAAGUUAAUUAUGUgCCUGGUCCA | |
| AGGAACUGGCAU | |
| GGAUGCCAGUUCCUUGGACCguuuuagaGCUAGAAAUAGCAAGUUAAAAUAAGGCUAGUCCGU | 1116 |
| UAUCAACUUGAAAAAGUGGCACCGAGUCGgugcAAUACCUAAAGUUAAUUAUGUgCCUGGUCC | |
| AAGGAACU | |
| GGAUGCCAGUUCCUUGGACCguuuuagaGCUAGAAAUAGCAAGUUAAAAUAAGGCUAGUCCGU | 1117 |
| UAUCAACUUGAAAAAGUGGCACCGAGUCGgugcAAUACCUAAAGUUAAUUAUGUgCCUGGUCC | |
| AAGGAACUG | |
| GGAUGCCAGUUCCUUGGACCguuuuagaGCUAGAAAUAGCAAGUUAAAAUAAGGCUAGUCCGU | 1118 |
| UAUCAACUUGAAAAAGUGGCACCGAGUCGgugcAAUACCUAAAGUUAAUUAUGUgCCUGGUCC | |
| AAGGAACUGG | |
| GGAUGCCAGUUCCUUGGACCguuuuagaGCUAGAAAUAGCAAGUUAAAAUAAGGCUAGUCCGU | 1119 |
| UAUCAACUUGAAAAAGUGGCACCGAGUCGgugcAAUACCUAAAGUUAAUUAUGUgCCUGGUCC | |
| AAGGAACUGGC | |
| GGAUGCCAGUUCCUUGGACCguuuuagaGCUAGAAAUAGCAAGUUAAAAUAAGGCUAGUCCGU | 1120 |
| UAUCAACUUGAAAAAGUGGCACCGAGUCGgugcAAUACCUAAAGUUAAUUAUGUgCCUGGUCC | |
| AAGGAACUGGCA | |
| GGAUGCCAGUUCCUUGGACCguuuuagaGCUAGAAAUAGCAAGUUAAAAUAAGGCUAGUCCGU | 1121 |
| UAUCAACUUGAAAAAGUGGCACCGAGUCGgugcAAUACCUAAAGUUAAUUAUGUgCCUGGUCC | |
| AAGGAACUGGCAU | |
| GGAUGCCAGUUCCUUGGACCguuuuagaGCUAGAAAUAGCAAGUUAAAAUAAGGCUAGUCCGU | 1122 |
| UAUCAACUUGAAAAAGUGGCACCGAGUCGgugcAAAUACCUAAAGUUAAUUAUGUgCCUGGUC | |
| CAAGGAACU | |
| GGAUGCCAGUUCCUUGGACCguuuuagaGCUAGAAAUAGCAAGUUAAAAUAAGGCUAGUCCGU | 1123 |
| UAUCAACUUGAAAAAGUGGCACCGAGUCGgugcAAAUACCUAAAGUUAAUUAUGUgCCUGGUC | |
| CAAGGAACUG | |
| GGAUGCCAGUUCCUUGGACCguuuuagaGCUAGAAAUAGCAAGUUAAAAUAAGGCUAGUCCGU | 1124 |
| UAUCAACUUGAAAAAGUGGCACCGAGUCGgugcAAAUACCUAAAGUUAAUUAUGUgCCUGGUC | |
| CAAGGAACUGG | |
| GGAUGCCAGUUCCUUGGACCguuuuagaGCUAGAAAUAGCAAGUUAAAAUAAGGCUAGUCCGU | 1125 |
| UAUCAACUUGAAAAAGUGGCACCGAGUCGgugcAAAUACCUAAAGUUAAUUAUGUgCCUGGUC | |
| CAAGGAACUGGC | |
| GGAUGCCAGUUCCUUGGACCguuuuagaGCUAGAAAUAGCAAGUUAAAAUAAGGCUAGUCCGU | 1126 |
| UAUCAACUUGAAAAAGUGGCACCGAGUCGgugcAAAUACCUAAAGUUAAUUAUGUgCCUGGUC | |
| CAAGGAACUGGCA | |
| GGAUGCCAGUUCCUUGGACCguuuuagaGCUAGAAAUAGCAAGUUAAAAUAAGGCUAGUCCGU | 1127 |
| UAUCAACUUGAAAAAGUGGCACCGAGUCGgugcAAAUACCUAAAGUUAAUUAUGUgCCUGGUC | |
| CAAGGAACUGGCAU | |
| GGAUGCCAGUUCCUUGGACCguuuuagaGCUAGAAAUAGCAAGUUAAAAUAAGGCUAGUCCGU | 1128 |
| UAUCAACUUGAAAAAGUGGCACCGAGUCGgugcUAAAUACCUAAAGUUAAUUAUGUgCCUGGU | |
| CCAAGGAACU | |
| GGAUGCCAGUUCCUUGGACCguuuuagaGCUAGAAAUAGCAAGUUAAAAUAAGGCUAGUCCGU | 1129 |
| UAUCAACUUGAAAAAGUGGCACCGAGUCGgugcUAAAUACCUAAAGUUAAUUAUGUgCCUGGU | |
| CCAAGGAACUG | |
| GGAUGCCAGUUCCUUGGACCguuuuagaGCUAGAAAUAGCAAGUUAAAAUAAGGCUAGUCCGU | 1130 |
| UAUCAACUUGAAAAAGUGGCACCGAGUCGgugcUAAAUACCUAAAGUUAAUUAUGUgCCUGGU | |
| CCAAGGAACUGG | |
| GGAUGCCAGUUCCUUGGACCguuuuagaGCUAGAAAUAGCAAGUUAAAAUAAGGCUAGUCCGU | 1131 |
| UAUCAACUUGAAAAAGUGGCACCGAGUCGgugcUAAAUACCUAAAGUUAAUUAUGUgCCUGGU | |
| CCAAGGAACUGGC | |
| GGAUGCCAGUUCCUUGGACCguuuuagaGCUAGAAAUAGCAAGUUAAAAUAAGGCUAGUCCGU | 1132 |
| UAUCAACUUGAAAAAGUGGCACCGAGUCGgugcUAAAUACCUAAAGUUAAUUAUGUgCCUGGU | |
| CCAAGGAACUGGCA | |
| GGAUGCCAGUUCCUUGGACCguuuuagaGCUAGAAAUAGCAAGUUAAAAUAAGGCUAGUCCGU | 1133 |
| UAUCAACUUGAAAAAGUGGCACCGAGUCGgugcUAAAUACCUAAAGUUAAUUAUGUgCCUGGU | |
| CCAAGGAACUGGCAU | |
| GCUAGGGGAUGCCAGUUCCUguuuuagaGCUAGAAAUAGCAAGUUAAAAUAAGGCUAGUCCGU | 1134 |
| UAUCAACUUGAAAAAGUGGCACCGAGUCGgugcUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1135 |
| UAUCAACUUGAAAAAGUGGCACCGAGUCGgugcUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1136 |
| UAUCAACUUGAAAAAGUGGCACCGAGUCGgugcUgCCUGGUCCAAGGAACUGGCAUCCC | |
| GCUAGGGGAUGCCAGUUCCUguuuuagaGCUAGAAAUAGCAAGUUAAAAUAAGGCUAGUCCGU | 1137 |
| UAUCAACUUGAAAAAGUGGCACCGAGUCGgugcUgCCUGGUCCAAGGAACUGGCAUCCCC | |
| GCUAGGGGAUGCCAGUUCCUguuuuagaGCUAGAAAUAGCAAGUUAAAAUAAGGCUAGUCCGU | 1138 |
| UAUCAACUUGAAAAAGUGGCACCGAGUCGgugcUgCCUGGUCCAAGGAACUGGCAUCCCCU | |
| GCUAGGGGAUGCCAGUUCCUguuuuagaGCUAGAAAUAGCAAGUUAAAAUAAGGCUAGUCCGU | 1139 |
| UAUCAACUUGAAAAAGUGGCACCGAGUCGgugcUgCCUGGUCCAAGGAACUGGCAUCCCCUA | |
| GCUAGGGGAUGCCAGUUCCUguuuuagaGCUAGAAAUAGCAAGUUAAAAUAAGGCUAGUCCGU | 1140 |
| UAUCAACUUGAAAAAGUGGCACCGAGUCGgugcGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1141 |
| UAUCAACUUGAAAAAGUGGCACCGAGUCGgugcGUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1142 |
| UAUCAACUUGAAAAAGUGGCACCGAGUCGgugcGUgCCUGGUCCAAGGAACUGGCAUCCC | |
| GCUAGGGGAUGCCAGUUCCUguuuuagaGCUAGAAAUAGCAAGUUAAAAUAAGGCUAGUCCGU | 1143 |
| UAUCAACUUGAAAAAGUGGCACCGAGUCGgugcGUgCCUGGUCCAAGGAACUGGCAUCCCC | |
| GCUAGGGGAUGCCAGUUCCUguuuuagaGCUAGAAAUAGCAAGUUAAAAUAAGGCUAGUCCGU | 1144 |
| UAUCAACUUGAAAAAGUGGCACCGAGUCGgugcGUgCCUGGUCCAAGGAACUGGCAUCCCCU | |
| GCUAGGGGAUGCCAGUUCCUguuuuagaGCUAGAAAUAGCAAGUUAAAAUAAGGCUAGUCCGU | 1145 |
| UAUCAACUUGAAAAAGUGGCACCGAGUCGgugcGUgCCUGGUCCAAGGAACUGGCAUCCCCUA | |
| GCUAGGGGAUGCCAGUUCCUguuuuagaGCUAGAAAUAGCAAGUUAAAAUAAGGCUAGUCCGU | 1146 |
| UAUCAACUUGAAAAAGUGGCACCGAGUCGgugcUGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1147 |
| UAUCAACUUGAAAAAGUGGCACCGAGUCGgugcUGUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1148 |
| UAUCAACUUGAAAAAGUGGCACCGAGUCGgugcUGUgCCUGGUCCAAGGAACUGGCAUCCC | |
| GCUAGGGGAUGCCAGUUCCUguuuuagaGCUAGAAAUAGCAAGUUAAAAUAAGGCUAGUCCGU | 1149 |
| UAUCAACUUGAAAAAGUGGCACCGAGUCGgugcUGUgCCUGGUCCAAGGAACUGGCAUCCCC | |
| GCUAGGGGAUGCCAGUUCCUguuuuagaGCUAGAAAUAGCAAGUUAAAAUAAGGCUAGUCCGU | 1150 |
| UAUCAACUUGAAAAAGUGGCACCGAGUCGgugcUGUgCCUGGUCCAAGGAACUGGCAUCCCCU | |
| GCUAGGGGAUGCCAGUUCCUguuuuagaGCUAGAAAUAGCAAGUUAAAAUAAGGCUAGUCCGU | 1151 |
| UAUCAACUUGAAAAAGUGGCACCGAGUCGgugcUGUgCCUGGUCCAAGGAACUGGCAUCCCCU | |
| A | |
| GCUAGGGGAUGCCAGUUCCUguuuuagaGCUAGAAAUAGCAAGUUAAAAUAAGGCUAGUCCGU | 1152 |
| UAUCAACUUGAAAAAGUGGCACCGAGUCGgugcAUGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1153 |
| UAUCAACUUGAAAAAGUGGCACCGAGUCGgugcAUGUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1154 |
| UAUCAACUUGAAAAAGUGGCACCGAGUCGgugcAUGUgCCUGGUCCAAGGAACUGGCAUCCC | |
| GCUAGGGGAUGCCAGUUCCUguuuuagaGCUAGAAAUAGCAAGUUAAAAUAAGGCUAGUCCGU | 1155 |
| UAUCAACUUGAAAAAGUGGCACCGAGUCGgugcAUGUgCCUGGUCCAAGGAACUGGCAUCCCC | |
| GCUAGGGGAUGCCAGUUCCUguuuuagaGCUAGAAAUAGCAAGUUAAAAUAAGGCUAGUCCGU | 1156 |
| UAUCAACUUGAAAAAGUGGCACCGAGUCGgugcAUGUgCCUGGUCCAAGGAACUGGCAUCCCC | |
| U | |
| GCUAGGGGAUGCCAGUUCCUguuuuagaGCUAGAAAUAGCAAGUUAAAAUAAGGCUAGUCCGU | 1157 |
| UAUCAACUUGAAAAAGUGGCACCGAGUCGgugcAUGUgCCUGGUCCAAGGAACUGGCAUCCCC | |
| UA | |
| GCUAGGGGAUGCCAGUUCCUguuuuagaGCUAGAAAUAGCAAGUUAAAAUAAGGCUAGUCCGU | 1158 |
| UAUCAACUUGAAAAAGUGGCACCGAGUCGgugcUAUGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1159 |
| UAUCAACUUGAAAAAGUGGCACCGAGUCGgugcUAUGUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1160 |
| UAUCAACUUGAAAAAGUGGCACCGAGUCGgugcUAUGUgCCUGGUCCAAGGAACUGGCAUCCC | |
| GCUAGGGGAUGCCAGUUCCUguuuuagaGCUAGAAAUAGCAAGUUAAAAUAAGGCUAGUCCGU | 1161 |
| UAUCAACUUGAAAAAGUGGCACCGAGUCGgugcUAUGUgCCUGGUCCAAGGAACUGGCAUCCC | |
| C | |
| GCUAGGGGAUGCCAGUUCCUguuuuagaGCUAGAAAUAGCAAGUUAAAAUAAGGCUAGUCCGU | 1162 |
| UAUCAACUUGAAAAAGUGGCACCGAGUCGgugcUAUGUgCCUGGUCCAAGGAACUGGCAUCCC | |
| CU | |
| GCUAGGGGAUGCCAGUUCCUguuuuagaGCUAGAAAUAGCAAGUUAAAAUAAGGCUAGUCCGU | 1163 |
| UAUCAACUUGAAAAAGUGGCACCGAGUCGgugcUAUGUgCCUGGUCCAAGGAACUGGCAUCCC | |
| CUA | |
| GCUAGGGGAUGCCAGUUCCUguuuuagaGCUAGAAAUAGCAAGUUAAAAUAAGGCUAGUCCGU | 1164 |
| UAUCAACUUGAAAAAGUGGCACCGAGUCGgugcUUAUGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1165 |
| UAUCAACUUGAAAAAGUGGCACCGAGUCGgugcUUAUGUgCCUGGUCCAAGGAACUGGCAUCC | |
| GCUAGGGGAUGCCAGUUCCUguuuuagaGCUAGAAAUAGCAAGUUAAAAUAAGGCUAGUCCGU | 1166 |
| UAUCAACUUGAAAAAGUGGCACCGAGUCGgugcUUAUGUgCCUGGUCCAAGGAACUGGCAUCC | |
| C | |
| GCUAGGGGAUGCCAGUUCCUguuuuagaGCUAGAAAUAGCAAGUUAAAAUAAGGCUAGUCCGU | 1167 |
| UAUCAACUUGAAAAAGUGGCACCGAGUCGgugcUUAUGUgCCUGGUCCAAGGAACUGGCAUCC | |
| cc | |
| GCUAGGGGAUGCCAGUUCCUguuuuagaGCUAGAAAUAGCAAGUUAAAAUAAGGCUAGUCCGU | 1168 |
| UAUCAACUUGAAAAAGUGGCACCGAGUCGgugcUUAUGUgCCUGGUCCAAGGAACUGGCAUCC | |
| CCU | |
| GCUAGGGGAUGCCAGUUCCUguuuuagaGCUAGAAAUAGCAAGUUAAAAUAAGGCUAGUCCGU | 1169 |
| UAUCAACUUGAAAAAGUGGCACCGAGUCGgugcUUAUGUgCCUGGUCCAAGGAACUGGCAUCC | |
| CCUA | |
| GCUAGGGGAUGCCAGUUCCUguuuuagaGCUAGAAAUAGCAAGUUAAAAUAAGGCUAGUCCGU | 1170 |
| UAUCAACUUGAAAAAGUGGCACCGAGUCGgugcAUUAUGUgCCUGGUCCAAGGAACUGGCAUC | |
| GCUAGGGGAUGCCAGUUCCUguuuuagaGCUAGAAAUAGCAAGUUAAAAUAAGGCUAGUCCGU | 1171 |
| UAUCAACUUGAAAAAGUGGCACCGAGUCGgugcAUUAUGUgCCUGGUCCAAGGAACUGGCAUC | |
| C | |
| GCUAGGGGAUGCCAGUUCCUguuuuagaGCUAGAAAUAGCAAGUUAAAAUAAGGCUAGUCCGU | 1172 |
| UAUCAACUUGAAAAAGUGGCACCGAGUCGgugcAUUAUGUgCCUGGUCCAAGGAACUGGCAUC | |
| CC | |
| GCUAGGGGAUGCCAGUUCCUguuuuagaGCUAGAAAUAGCAAGUUAAAAUAAGGCUAGUCCGU | 1173 |
| UAUCAACUUGAAAAAGUGGCACCGAGUCGgugcAUUAUGUgCCUGGUCCAAGGAACUGGCAUC | |
| CCC | |
| GCUAGGGGAUGCCAGUUCCUguuuuagaGCUAGAAAUAGCAAGUUAAAAUAAGGCUAGUCCGU | 1174 |
| UAUCAACUUGAAAAAGUGGCACCGAGUCGgugcAUUAUGUgCCUGGUCCAAGGAACUGGCAUC | |
| CCCU | |
| GCUAGGGGAUGCCAGUUCCUguuuuagaGCUAGAAAUAGCAAGUUAAAAUAAGGCUAGUCCGU | 1175 |
| UAUCAACUUGAAAAAGUGGCACCGAGUCGgugcAUUAUGUgCCUGGUCCAAGGAACUGGCAUC | |
| CCCUA | |
| GCUAGGGGAUGCCAGUUCCUguuuuagaGCUAGAAAUAGCAAGUUAAAAUAAGGCUAGUCCGU | 1176 |
| UAUCAACUUGAAAAAGUGGCACCGAGUCGgugcAAUUAUGUgCCUGGUCCAAGGAACUGGCAU | |
| C | |
| GCUAGGGGAUGCCAGUUCCUguuuuagaGCUAGAAAUAGCAAGUUAAAAUAAGGCUAGUCCGU | 1177 |
| UAUCAACUUGAAAAAGUGGCACCGAGUCGgugcAAUUAUGUgCCUGGUCCAAGGAACUGGCAU | |
| CC | |
| GCUAGGGGAUGCCAGUUCCUguuuuagaGCUAGAAAUAGCAAGUUAAAAUAAGGCUAGUCCGU | 1178 |
| UAUCAACUUGAAAAAGUGGCACCGAGUCGgugcAAUUAUGUgCCUGGUCCAAGGAACUGGCAU | |
| CCC | |
| GCUAGGGGAUGCCAGUUCCUguuuuagaGCUAGAAAUAGCAAGUUAAAAUAAGGCUAGUCCGU | 1179 |
| UAUCAACUUGAAAAAGUGGCACCGAGUCGgugcAAUUAUGUgCCUGGUCCAAGGAACUGGCAU | |
| CCCC | |
| GCUAGGGGAUGCCAGUUCCUguuuuagaGCUAGAAAUAGCAAGUUAAAAUAAGGCUAGUCCGU | 1180 |
| UAUCAACUUGAAAAAGUGGCACCGAGUCGgugcAAUUAUGUgCCUGGUCCAAGGAACUGGCAU | |
| CCCCU | |
| GCUAGGGGAUGCCAGUUCCUguuuuagaGCUAGAAAUAGCAAGUUAAAAUAAGGCUAGUCCGU | 1181 |
| UAUCAACUUGAAAAAGUGGCACCGAGUCGgugcAAUUAUGUgCCUGGUCCAAGGAACUGGCAU | |
| CCCCUA | |
| GCUAGGGGAUGCCAGUUCCUguuuuagaGCUAGAAAUAGCAAGUUAAAAUAAGGCUAGUCCGU | 1182 |
| UAUCAACUUGAAAAAGUGGCACCGAGUCGgugcUAAUUAUGUgCCUGGUCCAAGGAACUGGCA | |
| UC | |
| GCUAGGGGAUGCCAGUUCCUguuuuagaGCUAGAAAUAGCAAGUUAAAAUAAGGCUAGUCCGU | 1183 |
| UAUCAACUUGAAAAAGUGGCACCGAGUCGgugcUAAUUAUGUgCCUGGUCCAAGGAACUGGCA | |
| UCC | |
| GCUAGGGGAUGCCAGUUCCUguuuuagaGCUAGAAAUAGCAAGUUAAAAUAAGGCUAGUCCGU | 1184 |
| UAUCAACUUGAAAAAGUGGCACCGAGUCGgugcUAAUUAUGUgCCUGGUCCAAGGAACUGGCA | |
| UCCC | |
| GCUAGGGGAUGCCAGUUCCUguuuuagaGCUAGAAAUAGCAAGUUAAAAUAAGGCUAGUCCGU | 1185 |
| UAUCAACUUGAAAAAGUGGCACCGAGUCGgugcUAAUUAUGUgCCUGGUCCAAGGAACUGGCA | |
| UCCCC | |
| GCUAGGGGAUGCCAGUUCCUguuuuagaGCUAGAAAUAGCAAGUUAAAAUAAGGCUAGUCCGU | 1186 |
| UAUCAACUUGAAAAAGUGGCACCGAGUCGgugcUAAUUAUGUgCCUGGUCCAAGGAACUGGCA | |
| UCCCCU | |
| GCUAGGGGAUGCCAGUUCCUguuuuagaGCUAGAAAUAGCAAGUUAAAAUAAGGCUAGUCCGU | 1187 |
| UAUCAACUUGAAAAAGUGGCACCGAGUCGgugcUAAUUAUGUgCCUGGUCCAAGGAACUGGCA | |
| UCCCCUA | |
| GCUAGGGGAUGCCAGUUCCUguuuuagaGCUAGAAAUAGCAAGUUAAAAUAAGGCUAGUCCGU | 1188 |
| UAUCAACUUGAAAAAGUGGCACCGAGUCGgugcUUAAUUAUGUgCCUGGUCCAAGGAACUGGC | |
| AUC | |
| GCUAGGGGAUGCCAGUUCCUguuuuagaGCUAGAAAUAGCAAGUUAAAAUAAGGCUAGUCCGU | 1189 |
| UAUCAACUUGAAAAAGUGGCACCGAGUCGgugcUUAAUUAUGUgCCUGGUCCAAGGAACUGGC | |
| AUCC | |
| GCUAGGGGAUGCCAGUUCCUguuuuagaGCUAGAAAUAGCAAGUUAAAAUAAGGCUAGUCCGU | 1190 |
| UAUCAACUUGAAAAAGUGGCACCGAGUCGgugcUUAAUUAUGUgCCUGGUCCAAGGAACUGGC | |
| AUCCC | |
| GCUAGGGGAUGCCAGUUCCUguuuuagaGCUAGAAAUAGCAAGUUAAAAUAAGGCUAGUCCGU | 1191 |
| UAUCAACUUGAAAAAGUGGCACCGAGUCGgugcUUAAUUAUGUgCCUGGUCCAAGGAACUGGC | |
| AUCCCC | |
| GCUAGGGGAUGCCAGUUCCUguuuuagaGCUAGAAAUAGCAAGUUAAAAUAAGGCUAGUCCGU | 1192 |
| UAUCAACUUGAAAAAGUGGCACCGAGUCGgugcUUAAUUAUGUgCCUGGUCCAAGGAACUGGC | |
| AUCCCCU | |
| GCUAGGGGAUGCCAGUUCCUguuuuagaGCUAGAAAUAGCAAGUUAAAAUAAGGCUAGUCCGU | 1193 |
| UAUCAACUUGAAAAAGUGGCACCGAGUCGgugcUUAAUUAUGUgCCUGGUCCAAGGAACUGGC | |
| AUCCCCUA | |
| GCUAGGGGAUGCCAGUUCCUguuuuagaGCUAGAAAUAGCAAGUUAAAAUAAGGCUAGUCCGU | 1194 |
| UAUCAACUUGAAAAAGUGGCACCGAGUCGgugcGUUAAUUAUGUgCCUGGUCCAAGGAACUGG | |
| CAUC | |
| GCUAGGGGAUGCCAGUUCCUguuuuagaGCUAGAAAUAGCAAGUUAAAAUAAGGCUAGUCCGU | 1195 |
| UAUCAACUUGAAAAAGUGGCACCGAGUCGgugcGUUAAUUAUGUgCCUGGUCCAAGGAACUGG | |
| CAUCC | |
| GCUAGGGGAUGCCAGUUCCUguuuuagaGCUAGAAAUAGCAAGUUAAAAUAAGGCUAGUCCGU | 1196 |
| UAUCAACUUGAAAAAGUGGCACCGAGUCGgugcGUUAAUUAUGUgCCUGGUCCAAGGAACUGG | |
| CAUCCC | |
| GCUAGGGGAUGCCAGUUCCUguuuuagaGCUAGAAAUAGCAAGUUAAAAUAAGGCUAGUCCGU | 1197 |
| UAUCAACUUGAAAAAGUGGCACCGAGUCGgugcGUUAAUUAUGUgCCUGGUCCAAGGAACUGG | |
| CAUCCCC | |
| GCUAGGGGAUGCCAGUUCCUguuuuagaGCUAGAAAUAGCAAGUUAAAAUAAGGCUAGUCCGU | 1198 |
| UAUCAACUUGAAAAAGUGGCACCGAGUCGgugcGUUAAUUAUGUgCCUGGUCCAAGGAACUGG | |
| CAUCCCCU | |
| GCUAGGGGAUGCCAGUUCCUguuuuagaGCUAGAAAUAGCAAGUUAAAAUAAGGCUAGUCCGU | 1199 |
| UAUCAACUUGAAAAAGUGGCACCGAGUCGgugcGUUAAUUAUGUgCCUGGUCCAAGGAACUGG | |
| CAUCCCCUA | |
| GCUAGGGGAUGCCAGUUCCUguuuuagaGCUAGAAAUAGCAAGUUAAAAUAAGGCUAGUCCGU | 1200 |
| UAUCAACUUGAAAAAGUGGCACCGAGUCGgugcAGUUAAUUAUGUgCCUGGUCCAAGGAACUG | |
| GCAUC | |
| GCUAGGGGAUGCCAGUUCCUguuuuagaGCUAGAAAUAGCAAGUUAAAAUAAGGCUAGUCCGU | 1201 |
| UAUCAACUUGAAAAAGUGGCACCGAGUCGgugcAGUUAAUUAUGUgCCUGGUCCAAGGAACUG | |
| GCAUCC | |
| GCUAGGGGAUGCCAGUUCCUguuuuagaGCUAGAAAUAGCAAGUUAAAAUAAGGCUAGUCCGU | 1202 |
| UAUCAACUUGAAAAAGUGGCACCGAGUCGgugcAGUUAAUUAUGUgCCUGGUCCAAGGAACUG | |
| GCAUCCC | |
| GCUAGGGGAUGCCAGUUCCUguuuuagaGCUAGAAAUAGCAAGUUAAAAUAAGGCUAGUCCGU | 1203 |
| UAUCAACUUGAAAAAGUGGCACCGAGUCGgugcAGUUAAUUAUGUgCCUGGUCCAAGGAACUG | |
| GCAUCCCC | |
| GCUAGGGGAUGCCAGUUCCUguuuuagaGCUAGAAAUAGCAAGUUAAAAUAAGGCUAGUCCGU | 1204 |
| UAUCAACUUGAAAAAGUGGCACCGAGUCGgugcAGUUAAUUAUGUgCCUGGUCCAAGGAACUG | |
| GCAUCCCCU | |
| GCUAGGGGAUGCCAGUUCCUguuuuagaGCUAGAAAUAGCAAGUUAAAAUAAGGCUAGUCCGU | 1205 |
| UAUCAACUUGAAAAAGUGGCACCGAGUCGgugcAGUUAAUUAUGUgCCUGGUCCAAGGAACUG | |
| GCAUCCCCUA | |
| GCUAGGGGAUGCCAGUUCCUguuuuagaGCUAGAAAUAGCAAGUUAAAAUAAGGCUAGUCCGU | 1206 |
| UAUCAACUUGAAAAAGUGGCACCGAGUCGgugcAAGUUAAUUAUGUgCCUGGUCCAAGGAACU | |
| GGCAUC | |
| GCUAGGGGAUGCCAGUUCCUguuuuagaGCUAGAAAUAGCAAGUUAAAAUAAGGCUAGUCCGU | 1207 |
| UAUCAACUUGAAAAAGUGGCACCGAGUCGgugcAAGUUAAUUAUGUgCCUGGUCCAAGGAACU | |
| GGCAUCC | |
| GCUAGGGGAUGCCAGUUCCUguuuuagaGCUAGAAAUAGCAAGUUAAAAUAAGGCUAGUCCGU | 1208 |
| UAUCAACUUGAAAAAGUGGCACCGAGUCGgugcAAGUUAAUUAUGUgCCUGGUCCAAGGAACU | |
| GGCAUCCC | |
| GCUAGGGGAUGCCAGUUCCUguuuuagaGCUAGAAAUAGCAAGUUAAAAUAAGGCUAGUCCGU | 1209 |
| UAUCAACUUGAAAAAGUGGCACCGAGUCGgugcAAGUUAAUUAUGUgCCUGGUCCAAGGAACU | |
| GGCAUCCCC | |
| GCUAGGGGAUGCCAGUUCCUguuuuagaGCUAGAAAUAGCAAGUUAAAAUAAGGCUAGUCCGU | 1210 |
| UAUCAACUUGAAAAAGUGGCACCGAGUCGgugcAAGUUAAUUAUGUgCCUGGUCCAAGGAACU | |
| GGCAUCCCCU | |
| GCUAGGGGAUGCCAGUUCCUguuuuagaGCUAGAAAUAGCAAGUUAAAAUAAGGCUAGUCCGU | 1211 |
| UAUCAACUUGAAAAAGUGGCACCGAGUCGgugcAAGUUAAUUAUGUgCCUGGUCCAAGGAACU | |
| GGCAUCCCCUA | |
| GCUAGGGGAUGCCAGUUCCUguuuuagaGCUAGAAAUAGCAAGUUAAAAUAAGGCUAGUCCGU | 1212 |
| UAUCAACUUGAAAAAGUGGCACCGAGUCGgugcAAAGUUAAUUAUGUgCCUGGUCCAAGGAAC | |
| UGGCAUC | |
| GCUAGGGGAUGCCAGUUCCUguuuuagaGCUAGAAAUAGCAAGUUAAAAUAAGGCUAGUCCGU | 1213 |
| UAUCAACUUGAAAAAGUGGCACCGAGUCGgugcAAAGUUAAUUAUGUgCCUGGUCCAAGGAAC | |
| UGGCAUCC | |
| GCUAGGGGAUGCCAGUUCCUguuuuagaGCUAGAAAUAGCAAGUUAAAAUAAGGCUAGUCCGU | 1214 |
| UAUCAACUUGAAAAAGUGGCACCGAGUCGgugcAAAGUUAAUUAUGUgCCUGGUCCAAGGAAC | |
| UGGCAUCCC | |
| GCUAGGGGAUGCCAGUUCCUguuuuagaGCUAGAAAUAGCAAGUUAAAAUAAGGCUAGUCCGU | 1215 |
| UAUCAACUUGAAAAAGUGGCACCGAGUCGgugcAAAGUUAAUUAUGUgCCUGGUCCAAGGAAC | |
| UGGCAUCCCC | |
| GCUAGGGGAUGCCAGUUCCUguuuuagaGCUAGAAAUAGCAAGUUAAAAUAAGGCUAGUCCGU | 1216 |
| UAUCAACUUGAAAAAGUGGCACCGAGUCGgugcAAAGUUAAUUAUGUgCCUGGUCCAAGGAAC | |
| UGGCAUCCCCU | |
| GCUAGGGGAUGCCAGUUCCUguuuuagaGCUAGAAAUAGCAAGUUAAAAUAAGGCUAGUCCGU | 1217 |
| UAUCAACUUGAAAAAGUGGCACCGAGUCGgugcAAAGUUAAUUAUGUgCCUGGUCCAAGGAAC | |
| UGGCAUCCCCUA | |
| GCUAGGGGAUGCCAGUUCCUguuuuagaGCUAGAAAUAGCAAGUUAAAAUAAGGCUAGUCCGU | 1218 |
| UAUCAACUUGAAAAAGUGGCACCGAGUCGgugcUAAAGUUAAUUAUGUgCCUGGUCCAAGGAA | |
| CUGGCAUC | |
| GCUAGGGGAUGCCAGUUCCUguuuuagaGCUAGAAAUAGCAAGUUAAAAUAAGGCUAGUCCGU | 1219 |
| UAUCAACUUGAAAAAGUGGCACCGAGUCGgugcUAAAGUUAAUUAUGUgCCUGGUCCAAGGAA | |
| CUGGCAUCC | |
| GCUAGGGGAUGCCAGUUCCUguuuuagaGCUAGAAAUAGCAAGUUAAAAUAAGGCUAGUCCGU | 1220 |
| UAUCAACUUGAAAAAGUGGCACCGAGUCGgugcUAAAGUUAAUUAUGUgCCUGGUCCAAGGAA | |
| CUGGCAUCCC | |
| GCUAGGGGAUGCCAGUUCCUguuuuagaGCUAGAAAUAGCAAGUUAAAAUAAGGCUAGUCCGU | 1221 |
| UAUCAACUUGAAAAAGUGGCACCGAGUCGgugcUAAAGUUAAUUAUGUgCCUGGUCCAAGGAA | |
| CUGGCAUCCCC | |
| GCUAGGGGAUGCCAGUUCCUguuuuagaGCUAGAAAUAGCAAGUUAAAAUAAGGCUAGUCCGU | 1222 |
| UAUCAACUUGAAAAAGUGGCACCGAGUCGgugcUAAAGUUAAUUAUGUgCCUGGUCCAAGGAA | |
| CUGGCAUCCCCU | |
| GCUAGGGGAUGCCAGUUCCUguuuuagaGCUAGAAAUAGCAAGUUAAAAUAAGGCUAGUCCGU | 1223 |
| UAUCAACUUGAAAAAGUGGCACCGAGUCGgugcUAAAGUUAAUUAUGUgCCUGGUCCAAGGAA | |
| CUGGCAUCCCCUA | |
| GCUAGGGGAUGCCAGUUCCUguuuuagaGCUAGAAAUAGCAAGUUAAAAUAAGGCUAGUCCGU | 1224 |
| UAUCAACUUGAAAAAGUGGCACCGAGUCGgugcCUAAAGUUAAUUAUGUgCCUGGUCCAAGGA | |
| ACUGGCAUC | |
| GCUAGGGGAUGCCAGUUCCUguuuuagaGCUAGAAAUAGCAAGUUAAAAUAAGGCUAGUCCGU | 1225 |
| UAUCAACUUGAAAAAGUGGCACCGAGUCGgugcCUAAAGUUAAUUAUGUgCCUGGUCCAAGGA | |
| ACUGGCAUCC | |
| GCUAGGGGAUGCCAGUUCCUguuuuagaGCUAGAAAUAGCAAGUUAAAAUAAGGCUAGUCCGU | 1226 |
| UAUCAACUUGAAAAAGUGGCACCGAGUCGgugcCUAAAGUUAAUUAUGUgCCUGGUCCAAGGA | |
| ACUGGCAUCCC | |
| GCUAGGGGAUGCCAGUUCCUguuuuagaGCUAGAAAUAGCAAGUUAAAAUAAGGCUAGUCCGU | 1227 |
| UAUCAACUUGAAAAAGUGGCACCGAGUCGgugcCUAAAGUUAAUUAUGUgCCUGGUCCAAGGA | |
| ACUGGCAUCCCC | |
| GCUAGGGGAUGCCAGUUCCUguuuuagaGCUAGAAAUAGCAAGUUAAAAUAAGGCUAGUCCGU | 1228 |
| UAUCAACUUGAAAAAGUGGCACCGAGUCGgugcCUAAAGUUAAUUAUGUgCCUGGUCCAAGGA | |
| ACUGGCAUCCCCU | |
| GCUAGGGGAUGCCAGUUCCUguuuuagaGCUAGAAAUAGCAAGUUAAAAUAAGGCUAGUCCGU | 1229 |
| UAUCAACUUGAAAAAGUGGCACCGAGUCGgugcCUAAAGUUAAUUAUGUgCCUGGUCCAAGGA | |
| ACUGGCAUCCCCUA | |
| GCUAGGGGAUGCCAGUUCCUguuuuagaGCUAGAAAUAGCAAGUUAAAAUAAGGCUAGUCCGU | 1230 |
| UAUCAACUUGAAAAAGUGGCACCGAGUCGgugcCCUAAAGUUAAUUAUGUgCCUGGUCCAAGG | |
| AACUGGCAUC | |
| GCUAGGGGAUGCCAGUUCCUguuuuagaGCUAGAAAUAGCAAGUUAAAAUAAGGCUAGUCCGU | 1231 |
| UAUCAACUUGAAAAAGUGGCACCGAGUCGgugcCCUAAAGUUAAUUAUGUgCCUGGUCCAAGG | |
| AACUGGCAUCC | |
| GCUAGGGGAUGCCAGUUCCUguuuuagaGCUAGAAAUAGCAAGUUAAAAUAAGGCUAGUCCGU | 1232 |
| UAUCAACUUGAAAAAGUGGCACCGAGUCGgugcCCUAAAGUUAAUUAUGUgCCUGGUCCAAGG | |
| AACUGGCAUCCC | |
| GCUAGGGGAUGCCAGUUCCUguuuuagaGCUAGAAAUAGCAAGUUAAAAUAAGGCUAGUCCGU | 1233 |
| UAUCAACUUGAAAAAGUGGCACCGAGUCGgugcCCUAAAGUUAAUUAUGUgCCUGGUCCAAGG | |
| AACUGGCAUCCCC | |
| GCUAGGGGAUGCCAGUUCCUguuuuagaGCUAGAAAUAGCAAGUUAAAAUAAGGCUAGUCCGU | 1234 |
| UAUCAACUUGAAAAAGUGGCACCGAGUCGgugcCCUAAAGUUAAUUAUGUgCCUGGUCCAAGG | |
| AACUGGCAUCCCCU | |
| GCUAGGGGAUGCCAGUUCCUguuuuagaGCUAGAAAUAGCAAGUUAAAAUAAGGCUAGUCCGU | 1235 |
| UAUCAACUUGAAAAAGUGGCACCGAGUCGgugcCCUAAAGUUAAUUAUGUgCCUGGUCCAAGG | |
| AACUGGCAUCCCCUA | |

TABLE 13
Nicking RNA (nRNA) sequences.

| Represent | | | | | |
|---|---|---|---|---|---|
| ative | | | | | |
| spacer of | | | | | |
| a pegRNA | | | | | |
| with which | | | | | |
| the nRNA | | | | nickRNA | |
| may be | SEQ ID | | SEQ ID | (nRNA) | SEQ ID |
| used | NO | nRNA sequence | NO | spacer | NO |
| CCUAAAG | 1236 | caccGUAAUUCUAAAAGUAUGAUGUagaGC | 1308 | UAAUUCU | 1380 |
| UUAAUUA | | UAGAAAUAGCAAGUUAAAAUAAGGCUAGUC | | AAAAGUA | |
| UGUACC | | CGUUAUCAACUUGAAAAAGUGGCACCGAGU | | UGAUGU | |
| | | CG | | | |
| CCUAAAG | 1237 | caccGACUUUAGGUAUUUAUUCUGCagaGC | 1309 | ACUUUAG | 1381 |
| UUAAUUA | | UAGAAAUAGCAAGUUAAAAUAAGGCUAGUC | | GUAUUUA | |
| UGUACC | | CGUUAUCAACUUGAAAAAGUGGCACCGAGU | | UUCUGC | |
| | | CG | | | |
| CCUAAAG | 1238 | caccGCCAGGCACAUAAUUAACUUUagaGC | 1310 | GCCAGGA | 1382 |
| UUAAUUA | | UAGAAAUAGCAAGUUAAAAUAAGGCUAGUC | | CAUAAUU | |
| UGUACC | | CGUUAUCAACUUGAAAAAGUGGCACCGAGU | | AACUUU | |
| | | CG | | | |
| CCUAAAG | 1239 | caccGGAUGCCAGUUCCUUGGACCagaGCU | 1311 | GGAUGCC | 1383 |
| UUAAUUA | | AGAAAUAGCAAGUUAAAAUAAGGCUAGUCC | | AGUUCCU | |
| UGUACC | | GUUAUCAACUUGAAAAAGUGGCACCGAGUC | | UGGACC | |
| | | G | | | |
| CCUAAAG | 1240 | caccGCUAGGGGAUGCCAGUUCCUagaGCU | 1312 | GCUAGGG | 1384 |
| UUAAUUA | | AGAAAUAGCAAGUUAAAAUAAGGCUAGUCC | | GAUGCCA | |
| UGUACC | | GUUAUCAACUUGAAAAAGUGGCACCGAGUC | | GUUCCU | |
| | | G | | | |
| CCUAAAG | 1241 | caccGUGGGCAGCAGUAAGGGCUAGagaGC | 1313 | UGGGCAG | 1385 |
| UUAAUUA | | UAGAAAUAGCAAGUUAAAAUAAGGCUAGUC | | CAGUAAG | |
| UGUACC | | CGUUAUCAACUUGAAAAAGUGGCACCGAGU | | GGCUAG | |
| | | CG | | | |
| CCUAAAG | 1242 | caccGUUGGGCAGCAGUAAGGGCUAagaGC | 1314 | UUGGGCA | 1386 |
| UUAAUUA | | UAGAAAUAGCAAGUUAAAAUAAGGCUAGUC | | GCAGUAA | |
| UGUACC | | CGUUAUCAACUUGAAAAAGUGGCACCGAGU | | GGGCUA | |
| | | CG | | | |
| CCUAAAG | 1243 | caccGUUUGGGCAGCAGUAAGGGCUagaGC | 1315 | UUUGGGC | 1387 |
| UUAAUUA | | UAGAAAUAGCAAGUUAAAAUAAGGCUAGUC | | AGCAGUA | |
| UGUACC | | CGUUAUCAACUUGAAAAAGUGGCACCGAGU | | AGGGCU | |
| | | CG | | | |
| CCUAAAG | 1244 | caccGCAAUCUUUGGGCAGCAGUAAagaGC | 1316 | CAAUCUU | 1388 |
| UUAAUUA | | UAGAAAUAGCAAGUUAAAAUAAGGCUAGUC | | UGGGCAG | |
| UGUACC | | CGUUAUCAACUUGAAAAAGUGGCACCGAGU | | CAGUAA | |
| | | CG | | | |
| CCUAAAG | 1245 | caccGCCAAUCUUUGGGCAGCAGUAagaGC | 1317 | CCAAUCU | 1389 |
| UUAAUUA | | UAGAAAUAGCAAGUUAAAAUAAGGCUAGUC | | UUGGGCA | |
| UGUACC | | CGUUAUCAACUUGAAAAAGUGGCACCGAGU | | GCAGUA | |
| | | CG | | | |
| CCUAAAG | 1246 | caccGCAACUCUCUUCCAAUCUUUagaGCU | 1318 | GCAACUC | 1390 |
| UUAAUUA | | AGAAAUAGCAAGUUAAAAUAAGGCUAGUCC | | UCUUCCA | |
| UGUACC | | GUUAUCAACUUGAAAAAGUGGCACCGAGUC | | AUCUUU | |
| | | G | | | |
| CCUAAAG | 1247 | caccGAGCAACUCUCUUCCAAUCUUagaGC | 1319 | AGCAACU | 1391 |
| UUAAUUA | | UAGAAAUAGCAAGUUAAAAUAAGGCUAGUC | | CUCUUCC | |
| UGUACC | | CGUUAUCAACUUGAAAAAGUGGCACCGAGU | | AAUCUU | |
| | | CG | | | |
| GGAUGCC | 1248 | caccGCCUAAAGUUAAUUAUGUgCCagaGC | 1320 | CCUAAAG | 1392 |
| AGUUCCU | | UAGAAAUAGCAAGUUAAAAUAAGGCUAGUC | | UUAAUUA | |
| UGGACC | | CGUUAUCAACUUGAAAAAGUGGCACCGAGU | | UGUgCC | |
| | | CG | | | |
| GGAUGCC | 1249 | caccGUUAAUUAUGUgCCUGGUCCAagaGC | 1321 | UUAAUUA | 1393 |
| AGUUCCU | | UAGAAAUAGCAAGUUAAAAUAAGGCUAGUC | | UGUgCCU | |
| UGGACC | | CGUUAUCAACUUGAAAAAGUGGCACCGAGU | | GGUCCA | |
| | | CG | | | |
| GGAUGCC | 1250 | caccGAUGUgCCUGGUCCAAGGAACagaGC | 1322 | AUGUgCC | 1394 |
| AGUUCCU | | UAGAAAUAGCAAGUUAAAAUAAGGCUAGUC | | UGGUCCA | |
| UGGACC | | CGUUAUCAACUUGAAAAAGUGGCACCGAGU | | AGGAAC | |
| | | CG | | | |
| GGAUGCC | 1251 | caccGCCUUACUGCUGCCCAAAGAUagaGC | 1323 | CCUUACU | 1395 |
| AGUUCCU | | UAGAAAUAGCAAGUUAAAAUAAGGCUAGUC | | GCUGCCC | |
| UGGACC | | CGUUAUCAACUUGAAAAAGUGGCACCGAGU | | AAAGAU | |
| | | CG | | | |
| GGAUGCC | 1252 | caccGAGAUUGGAAGAGAGUUGCUGagaGC | 1324 | AGAUUGG | 1396 |
| AGUUCCU | | UAGAAAUAGCAAGUUAAAAUAAGGCUAGUC | | AAGAGAG | |
| UGGACC | | CGUUAUCAACUUGAAAAAGUGGCACCGAGU | | UUGCUG | |
| | | CG | | | |
| GGAUGCC | 1253 | caccGAAGAGAGUUGCUGAGGAUUCagaGC | 1325 | AAGAGAG | 1397 |
| AGUUCCU | | UAGAAAUAGCAAGUUAAAAUAAGGCUAGUC | | UUGCUGA | |
| UGGACC | | CGUUAUCAACUUGAAAAAGUGGCACCGAGU | | GGAUUC | |
| | | CG | | | |
| GCUAGGG | 1254 | caccGCCUAAAGUUAAUUAUGUgCCagaGC | 1326 | CCUAAAG | 1398 |
| GAUGCCA | | UAGAAAUAGCAAGUUAAAAUAAGGCUAGUC | | UUAAUUA | |
| GUUCCU | | CGUUAUCAACUUGAAAAAGUGGCACCGAGU | | UGUgCC | |
| | | CG | | | |
| GCUAGGG | 1255 | caccGUUAAUUAUGUgCCUGGUCCAagaGC | 1327 | UUAAUUA | 1399 |
| GAUGCCA | | UAGAAAUAGCAAGUUAAAAUAAGGCUAGUC | | UGUgCCU | |
| GUUCCU | | CGUUAUCAACUUGAAAAAGUGGCACCGAGU | | GGUCCA | |
| | | CG | | | |
| GCUAGGG | 1256 | caccGAUGUgCCUGGUCCAAGGAACagaGC | 1328 | AUGUgCC | 1400 |
| GAUGCCA | | UAGAAAUAGCAAGUUAAAAUAAGGCUAGUC | | UGGUCCA | |
| GUUCCU | | CGUUAUCAACUUGAAAAAGUGGCACCGAGU | | AGGAAC | |
| | | CG | | | |
| GCUAGGG | 1257 | caccGCCUUACUGCUGCCCAAAGAUagaGC | 1329 | CCUUACU | 1401 |
| GAUGCCA | | UAGAAAUAGCAAGUUAAAAUAAGGCUAGUC | | GCUGCCC | |
| GUUCCU | | CGUUAUCAACUUGAAAAAGUGGCACCGAGU | | AAAGAU | |
| | | CG | | | |
| GCUAGGG | 1258 | caccGAGAUUGGAAGAGAGUUGCUGagaGC | 1330 | AGAUUGG | 1402 |
| GAUGCCA | | UAGAAAUAGCAAGUUAAAAUAAGGCUAGUC | | AAGAGAG | |
| GUUCCU | | CGUUAUCAACUUGAAAAAGUGGCACCGAGU | | UUGCUG | |
| | | CG | | | |
| GCUAGGG | 1259 | caccGAAGAGAGUUGCUGAGGAUUCagaGC | 1331 | AAGAGAG | 1403 |
| GAUGCCA | | UAGAAAUAGCAAGUUAAAAUAAGGCUAGUC | | UUGCUGA | |
| GUUCCU | | CGUUAUCAACUUGAAAAAGUGGCACCGAGU | | GGAUUC | |
| | | CG | | | |
| UGGGCAG | 1260 | caccGCCUAAAGUUAAUUAUGUgCCagaGC | 1332 | CCUAAAG | 1404 |
| CAGUAAG | | UAGAAAUAGCAAGUUAAAAUAAGGCUAGUC | | UUAAUUA | |
| GGCUAG | | CGUUAUCAACUUGAAAAAGUGGCACCGAGU | | UGUgCC | |
| | | CG | | | |
| UGGGCAG | 1261 | caccGUUAAUUAUGUgCCUGGUCCAagaGC | 1333 | UUAAUUA | 1405 |
| CAGUAAG | | UAGAAAUAGCAAGUUAAAAUAAGGCUAGUC | | UGUgCCU | |
| GGCUAG | | CGUUAUCAACUUGAAAAAGUGGCACCGAGU | | GGUCCA | |
| | | CG | | | |
| UGGGCAG | 1262 | caccGAUGUgCCUGGUCCAAGGAACagaGC | 1334 | AUGUgCC | 1406 |
| CAGUAAG | | UAGAAAUAGCAAGUUAAAAUAAGGCUAGUC | | UGGUCCA | |
| GGCUAG | | CGUUAUCAACUUGAAAAAGUGGCACCGAGU | | AGGAAC | |
| | | CG | | | |
| UGGGCAG | 1263 | caccGCCUUACUGCUGCCCAAAGAUagaGC | 1335 | CCUUACU | 1407 |
| CAGUAAG | | UAGAAAUAGCAAGUUAAAAUAAGGCUAGUC | | GCUGCCC | |
| GGCUAG | | CGUUAUCAACUUGAAAAAGUGGCACCGAGU | | AAAGAU | |
| | | CG | | | |
| UGGGCAG | 1264 | caccGAGAUUGGAAGAGAGUUGCUGagaGC | 1336 | AGAUUGG | 1408 |
| CAGUAAG | | UAGAAAUAGCAAGUUAAAAUAAGGCUAGUC | | AAGAGAG | |
| GGCUAG | | CGUUAUCAACUUGAAAAAGUGGCACCGAGU | | UUGCUG | |
| | | CG | | | |
| UGGGCAG | 1265 | caccGAAGAGAGUUGCUGAGGAUUCagaGC | 1337 | AAGAGAG | 1409 |
| CAGUAAG | | UAGAAAUAGCAAGUUAAAAUAAGGCUAGUC | | UUGCUGA | |
| GGCUAG | | CGUUAUCAACUUGAAAAAGUGGCACCGAGU | | GGAUUC | |
| | | CG | | | |
| UUGGGCA | 1266 | caccGCCUAAAGUUAAUUAUGUgCCagaGC | 1338 | CCUAAAG | 1410 |
| GCAGUAA | | UAGAAAUAGCAAGUUAAAAUAAGGCUAGUC | | UUAAUUA | |
| GGGCUA | | CGUUAUCAACUUGAAAAAGUGGCACCGAGU | | UGUgCC | |
| | | CG | | | |
| UUGGGCA | 1267 | caccGUUAAUUAUGUgCCUGGUCCAagaGC | 1339 | UUAAUUA | 1411 |
| GCAGUAA | | UAGAAAUAGCAAGUUAAAAUAAGGCUAGUC | | UGUgCCU | |
| GGGCUA | | CGUUAUCAACUUGAAAAAGUGGCACCGAGU | | GGUCCA | |
| | | CG | | | |
| UUGGGCA | 1268 | caccGAUGUgCCUGGUCCAAGGAACagaGC | 1340 | AUGUgCC | 1412 |
| GCAGUAA | | UAGAAAUAGCAAGUUAAAAUAAGGCUAGUC | | UGGUCCA | |
| GGGCUA | | CGUUAUCAACUUGAAAAAGUGGCACCGAGU | | AGGAAC | |
| | | CG | | | |
| UUGGGCA | 1269 | caccGCCUUACUGCUGCCCAAAGAUagaGC | 1341 | CCUUACU | 1413 |
| GCAGUAA | | UAGAAAUAGCAAGUUAAAAUAAGGCUAGUC | | GCUGCCC | |
| GGGCUA | | CGUUAUCAACUUGAAAAAGUGGCACCGAGU | | AAAGAU | |
| | | CG | | | |
| UUGGGCA | 1270 | caccGAGAUUGGAAGAGAGUUGCUGagaGC | 1342 | AGAUUGG | 1414 |
| GCAGUAA | | UAGAAAUAGCAAGUUAAAAUAAGGCUAGUC | | AAGAGAG | |
| GGGCUA | | CGUUAUCAACUUGAAAAAGUGGCACCGAGU | | UUGCUG | |
| | | CG | | | |
| UUGGGCA | 1271 | caccGAAGAGAGUUGCUGAGGAUUCagaGC | 1343 | AAGAGAG | 1415 |
| GCAGUAA | | UAGAAAUAGCAAGUUAAAAUAAGGCUAGUC | | UUGCUGA | |
| GGGCUA | | CGUUAUCAACUUGAAAAAGUGGCACCGAGU | | GGAUUC | |
| | | CG | | | |
| CCUAAAG | 1272 | UAAUUCUAAAAGUAUGAUGUGUUUUAGAGC | 1344 | UAAUUCU | 1416 |
| UUAAUUA | | UAGAAAUAGCAAGUUAAAAUAAGGCUAGUC | | AAAAGUA | |
| UGUACC | | CGUUAUCAACUUGAAAAAGUGGCACCGAGU | | UGAUGU | |
| | | CGGUGCUUUU | | | |
| CCUAAAG | 1273 | ACUUUAGGUAUUUAUUCUGCGUUUUAGAGC | 1345 | ACUUUAG | 1417 |
| UUAAUUA | | UAGAAAUAGCAAGUUAAAAUAAGGCUAGUC | | GUAUUUA | |
| UGUACC | | CGUUAUCAACUUGAAAAAGUGGCACCGAGU | | UUCUGC | |
| | | CGGUGCUUUU | | | |
| CCUAAAG | 1274 | GCCAGGACAUAAUUAACUUUGUUUUAGAGC | 1346 | GCCAGGA | 1418 |
| UUAAUUA | | UAGAAAUAGCAAGUUAAAAUAAGGCUAGUC | | CAUAAUU | |
| UGUACC | | CGUUAUCAACUUGAAAAAGUGGCACCGAGU | | AACUUU | |
| | | CGGUGCUUUU | | | |
| CCUAAAG | 1275 | GGAUGCCAGUUCCUUGGACCGUUUUAGAGC | 1347 | GGAUGCC | 1419 |
| UUAAUUA | | UAGAAAUAGCAAGUUAAAAUAAGGCUAGUC | | AGUUCCU | |
| UGUACC | | CGUUAUCAACUUGAAAAAGUGGCACCGAGU | | UGGACC | |
| | | CGGUGCUUUU | | | |
| CCUAAAG | 1276 | GCUAGGGGAUGCCAGUUCCUGUUUUAGAGC | 1348 | GCUAGGG | 1420 |
| UUAAUUA | | UAGAAAUAGCAAGUUAAAAUAAGGCUAGUC | | GAUGCCA | |
| UGUACC | | CGUUAUCAACUUGAAAAAGUGGCACCGAGU | | GUUCCU | |
| | | CGGUGCUUUU | | | |
| CCUAAAG | 1277 | UGGGCAGCAGUAAGGGCUAGGUUUUAGAGC | 1349 | UGGGCAG | 1421 |
| UUAAUUA | | UAGAAAUAGCAAGUUAAAAUAAGGCUAGUC | | CAGUAAG | |
| UGUACC | | CGUUAUCAACUUGAAAAAGUGGCACCGAGU | | GGCUAG | |
| | | CGGUGCUUUU | | | |
| CCUAAAG | 1278 | UUGGGCAGCAGUAAGGGCUAGUUUUAGAGC | 1350 | UUGGGCA | 1422 |
| UUAAUUA | | UAGAAAUAGCAAGUUAAAAUAAGGCUAGUC | | GCAGUAA | |
| UGUACC | | CGUUAUCAACUUGAAAAAGUGGCACCGAGU | | GGGCUA | |
| | | CGGUGCUUUU | | | |
| CCUAAAG | 1279 | UUUGGGCAGCAGUAAGGGCUGUUUUAGAGC | 1351 | UUUGGGC | 1423 |
| UUAAUUA | | UAGAAAUAGCAAGUUAAAAUAAGGCUAGUC | | AGCAGUA | |
| UGUACC | | CGUUAUCAACUUGAAAAAGUGGCACCGAGU | | AGGGCU | |
| | | CGGUGCUUUU | | | |
| CCUAAAG | 1280 | CAAUCUUUGGGCAGCAGUAAGUUUUAGAGC | 1352 | CAAUCUU | 1424 |
| UUAAUUA | | UAGAAAUAGCAAGUUAAAAUAAGGCUAGUC | | UGGGCAG | |
| UGUACC | | CGUUAUCAACUUGAAAAAGUGGCACCGAGU | | CAGUAA | |
| | | CGGUGCUUUU | | | |
| CCUAAAG | 1281 | CCAAUCUUUGGGCAGCAGUAGUUUUAGAGC | 1353 | CCAAUCU | 1425 |
| UUAAUUA | | UAGAAAUAGCAAGUUAAAAUAAGGCUAGUC | | UUGGGCA | |
| UGUACC | | CGUUAUCAACUUGAAAAAGUGGCACCGAGU | | GCAGUA | |
| | | CGGUGCUUUU | | | |
| CCUAAAG | 1282 | GCAACUCUCUUCCAAUCUUUGUUUUAGAGC | 1354 | GCAACUC | 1426 |
| UUAAUUA | | UAGAAAUAGCAAGUUAAAAUAAGGCUAGUC | | UCUUCCA | |
| UGUACC | | CGUUAUCAACUUGAAAAAGUGGCACCGAGU | | AUCUUU | |
| | | CGGUGCUUUU | | | |
| CCUAAAG | 1283 | AGCAACUCUCUUCCAAUCUUGUUUUAGAGC | 1355 | AGCAACU | 1427 |
| UUAAUUA | | UAGAAAUAGCAAGUUAAAAUAAGGCUAGUC | | CUCUUCC | |
| UGUACC | | CGUUAUCAACUUGAAAAAGUGGCACCGAGU | | AAUCUU | |
| | | CGGUGCUUUU | | | |
| GGAUGCC | 1284 | CCUAAAGUUAAUUAUGUgCCGUUUUAGAGC | 1356 | CCUAAAG | 1428 |
| AGUUCCU | | UAGAAAUAGCAAGUUAAAAUAAGGCUAGUC | | UUAAUUA | |
| UGGACC | | CGUUAUCAACUUGAAAAAGUGGCACCGAGU | | UGUgCC | |
| | | CGGUGCUUUU | | | |
| GGAUGCC | 1285 | UUAAUUAUGUgCCUGGUCCAGUUUUAGAGC | 1357 | UUAAUUA | 1429 |
| AGUUCCU | | UAGAAAUAGCAAGUUAAAAUAAGGCUAGUC | | UGUgCCU | |
| UGGACC | | CGUUAUCAACUUGAAAAAGUGGCACCGAGU | | GGUCCA | |
| | | CGGUGCUUUU | | | |
| GGAUGCC | 1286 | AUGUgCCUGGUCCAAGGAACGUUUUAGAGC | 1358 | AUGUgCC | 1430 |
| AGUUCCU | | UAGAAAUAGCAAGUUAAAAUAAGGCUAGUC | | UGGUCCA | |
| UGGACC | | CGUUAUCAACUUGAAAAAGUGGCACCGAGU | | AGGAAC | |
| | | CGGUGCUUUU | | | |
| GGAUGCC | 1287 | CCUUACUGCUGCCCAAAGAUGUUUUAGAGC | 1359 | CCUUACU | 1431 |
| AGUUCCU | | UAGAAAUAGCAAGUUAAAAUAAGGCUAGUC | | GCUGCCC | |
| UGGACC | | CGUUAUCAACUUGAAAAAGUGGCACCGAGU | | AAAGAU | |
| | | CGGUGCUUUU | | | |
| GGAUGCC | 1288 | AGAUUGGAAGAGAGUUGCUGGUUUUAGAGC | 1360 | AGAUUGG | 1432 |
| AGUUCCU | | UAGAAAUAGCAAGUUAAAAUAAGGCUAGUC | | AAGAGAG | |
| UGGACC | | CGUUAUCAACUUGAAAAAGUGGCACCGAGU | | UUGCUG | |
| | | CGGUGCUUUU | | | |
| GGAUGCC | 1289 | AAGAGAGUUGCUGAGGAUUCGUUUUAGAGC | 1361 | AAGAGAG | 1433 |
| AGUUCCU | | UAGAAAUAGCAAGUUAAAAUAAGGCUAGUC | | UUGCUGA | |
| UGGACC | | CGUUAUCAACUUGAAAAAGUGGCACCGAGU | | GGAUUC | |
| | | CGGUGCUUUU | | | |
| GCUAGGG | 1290 | CCUAAAGUUAAUUAUGUgCCGUUUUAGAGC | 1362 | CCUAAAG | 1434 |
| GAUGCCA | | UAGAAAUAGCAAGUUAAAAUAAGGCUAGUC | | UUAAUUA | |
| GUUCCU | | CGUUAUCAACUUGAAAAAGUGGCACCGAGU | | UGUgCC | |
| | | CGGUGCUUUU | | | |
| GCUAGGG | 1291 | UUAAUUAUGUgCCUGGUCCAGUUUUAGAGC | 1363 | UUAAUUA | 1435 |
| GAUGCCA | | UAGAAAUAGCAAGUUAAAAUAAGGCUAGUC | | UGUgCCU | |
| GUUCCU | | CGUUAUCAACUUGAAAAAGUGGCACCGAGU | | GGUCCA | |
| | | CGGUGCUUUU | | | |
| GCUAGGG | 1292 | AUGUgCCUGGUCCAAGGAACGUUUUAGAGC | 1364 | AUGUgCC | 1436 |
| GAUGCCA | | UAGAAAUAGCAAGUUAAAAUAAGGCUAGUC | | UGGUCCA | |
| GUUCCU | | CGUUAUCAACUUGAAAAAGUGGCACCGAGU | | AGGAAC | |
| | | CGGUGCUUUU | | | |
| GCUAGGG | 1293 | CCUUACUGCUGCCCAAAGAUGUUUUAGAGC | 1365 | CCUUACU | 1437 |
| GAUGCCA | | UAGAAAUAGCAAGUUAAAAUAAGGCUAGUC | | GCUGCCC | |
| GUUCCU | | CGUUAUCAACUUGAAAAAGUGGCACCGAGU | | AAAGAU | |
| | | CGGUGCUUUU | | | |
| GCUAGGG | 1294 | AGAUUGGAAGAGAGUUGCUGGUUUUAGAGC | 1366 | AGAUUGG | 1438 |
| GAUGCCA | | UAGAAAUAGCAAGUUAAAAUAAGGCUAGUC | | AAGAGAG | |
| GUUCCU | | CGUUAUCAACUUGAAAAAGUGGCACCGAGU | | UUGCUG | |
| | | CGGUGCUUUU | | | |
| GCUAGGG | 1295 | AAGAGAGUUGCUGAGGAUUCGUUUUAGAGC | 1367 | AAGAGAG | 1439 |
| GAUGCCA | | UAGAAAUAGCAAGUUAAAAUAAGGCUAGUC | | UUGCUGA | |
| GUUCCU | | CGUUAUCAACUUGAAAAAGUGGCACCGAGU | | GGAUUC | |
| | | CGGUGCUUUU | | | |
| UGGGCAG | 1296 | CCUAAAGUUAAUUAUGUgCCGUUUUAGAGC | 1368 | CCUAAAG | 1440 |
| CAGUAAG | | UAGAAAUAGCAAGUUAAAAUAAGGCUAGUC | | UUAAUUA | |
| GGCUAG | | CGUUAUCAACUUGAAAAAGUGGCACCGAGU | | UGUgCC | |
| | | CGGUGCUUUU | | | |
| UGGGCAG | 1297 | UUAAUUAUGUgCCUGGUCCAGUUUUAGAGC | 1369 | UUAAUUA | 1441 |
| CAGUAAG | | UAGAAAUAGCAAGUUAAAAUAAGGCUAGUC | | UGUgCCU | |
| GGCUAG | | CGUUAUCAACUUGAAAAAGUGGCACCGAGU | | GGUCCA | |
| | | CGGUGCUUUU | | | |
| UGGGCAG | 1298 | AUGUgCCUGGUCCAAGGAACGUUUUAGAGC | 1370 | AUGUgCC | 1442 |
| CAGUAAG | | UAGAAAUAGCAAGUUAAAAUAAGGCUAGUC | | UGGUCCA | |
| GGCUAG | | CGUUAUCAACUUGAAAAAGUGGCACCGAGU | | AGGAAC | |
| | | CGGUGCUUUU | | | |
| UGGGCAG | 1299 | CCUUACUGCUGCCCAAAGAUGUUUUAGAGC | 1371 | CCUUACU | 1443 |
| CAGUAAG | | UAGAAAUAGCAAGUUAAAAUAAGGCUAGUC | | GCUGCCC | |
| GGCUAG | | CGUUAUCAACUUGAAAAAGUGGCACCGAGU | | AAAGAU | |
| | | CGGUGCUUUU | | | |
| UGGGCAG | 1300 | AGAUUGGAAGAGAGUUGCUGGUUUUAGAGC | 1372 | AGAUUGG | 1444 |
| CAGUAAG | | UAGAAAUAGCAAGUUAAAAUAAGGCUAGUC | | AAGAGAG | |
| GGCUAG | | CGUUAUCAACUUGAAAAAGUGGCACCGAGU | | UUGCUG | |
| | | CGGUGCUUUU | | | |
| UGGGCAG | 1301 | AAGAGAGUUGCUGAGGAUUCGUUUUAGAGC | 1373 | AAGAGAG | 1445 |
| CAGUAAG | | UAGAAAUAGCAAGUUAAAAUAAGGCUAGUC | | UUGCUGA | |
| GGCUAG | | CGUUAUCAACUUGAAAAAGUGGCACCGAGU | | GGAUUC | |
| | | CGGUGCUUUU | | | |
| UUGGGCA | 1302 | CCUAAAGUUAAUUAUGUgCCGUUUUAGAGC | 1374 | CCUAAAG | 1446 |
| GCAGUAA | | UAGAAAUAGCAAGUUAAAAUAAGGCUAGUC | | UUAAUUA | |
| GGGCUA | | CGUUAUCAACUUGAAAAAGUGGCACCGAGU | | UGUgCC | |
| | | CGGUGCUUUU | | | |
| UUGGGCA | 1303 | UUAAUUAUGUgCCUGGUCCAGUUUUAGAGC | 1375 | UUAAUUA | 1447 |
| GCAGUAA | | UAGAAAUAGCAAGUUAAAAUAAGGCUAGUC | | UGUgCCU | |
| GGGCUA | | CGUUAUCAACUUGAAAAAGUGGCACCGAGU | | GGUCCA | |
| | | CGGUGCUUUU | | | |
| UUGGGCA | 1304 | AUGUgCCUGGUCCAAGGAACGUUUUAGAGC | 1376 | AUGUgCC | 1448 |
| GCAGUAA | | UAGAAAUAGCAAGUUAAAAUAAGGCUAGUC | | UGGUCCA | |
| GGGCUA | | CGUUAUCAACUUGAAAAAGUGGCACCGAGU | | AGGAAC | |
| | | CGGUGCUUUU | | | |
| UUGGGCA | 1305 | CCUUACUGCUGCCCAAAGAUGUUUUAGAGC | 1377 | CCUUACU | 1449 |
| GCAGUAA | | UAGAAAUAGCAAGUUAAAAUAAGGCUAGUC | | GCUGCCC | |
| GGGCUA | | CGUUAUCAACUUGAAAAAGUGGCACCGAGU | | AAAGAU | |
| | | CGGUGCUUUU | | | |
| UUGGGCA | 1306 | AGAUUGGAAGAGAGUUGCUGGUUUUAGAGC | 1378 | AGAUUGG | 1450 |
| GCAGUAA | | UAGAAAUAGCAAGUUAAAAUAAGGCUAGUC | | AAGAGAG | |
| GGGCUA | | CGUUAUCAACUUGAAAAAGUGGCACCGAGU | | UUGCUG | |
| | | CGGUGCUUUU | | | |
| UUGGGCA | 1307 | AAGAGAGUUGCUGAGGAUUCGUUUUAGAGC | 1379 | AAGAGAG | 1451 |
| GCAGUAA | | UAGAAAUAGCAAGUUAAAAUAAGGCUAGUC | | UUGCUGA | |
| GGGCUA | | CGUUAUCAACUUGAAAAAGUGGCACCGAGU | | GGAUUC | |
| | | CGGUGCUUUU | | | |

### OTHER EMBODIMENTS

From the foregoing description, it will be apparent that variations and modifications may be made to the embodiments and aspects described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The present disclosure may be related to one or more of International Patent Applications No. PCT/US22/75021, PCT/US20/13964, PCT/US20/52822, PCT/US20/18178, PCT/US21/52035, PCT/US22/81241, PCT/US23/67780, PCT/US23/68543, and/or PCT/US23/72911, and/or U.S. Provisional Patent Applications Nos. 63/592,339, 63/612,146, 63/618,520, and/or 63/621,953, the disclosures of which are incorporated herein by reference in their entirety for all purposes. All patents and publications mentioned in this specification are herein incorporated by reference to the same extent as if each independent patent and publication was specifically and individually indicated to be incorporated by reference.

What is claimed:

1. A method for producing a modified immune effector cell having reduced exhaustion after antigen exposure relative to an unedited immune effector cell, the method comprising contacting the cell with:
(i) a base editor, or a polynucleotide encoding the base editor, wherein the base editor comprises a programmable DNA binding domain and a deaminase domain;
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(iii) a guide polynucleotide, or a polynucleotide encoding the guide polynucleotide, wherein the guide polynucleotide directs the base editor to effect a nucleobase alteration in a polynucleotide encoding a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC);
wherein each nucleobase alteration effects a reduction in expression of the encoded polypeptide, thereby producing the modified immune effector cell.

2. The method of claim 1, further comprising expressing a chimeric antigen receptor in the cell.

3. The method of claim 2, further comprising contacting the cell with a guide polynucleotide, or a polynucleotide encoding the guide polynucleotide, wherein the guide polynucleotide directs the base editor to effect a nucleobase alteration in a polynucleotide encoding an antigen bound by the chimeric antigen receptor, wherein the nucleobase alteration effects a reduction in expression of the antigen.

4. The method of claim 1, wherein the base editing reduces expression of a set of polypeptides selected from:
a Roquin-1, DGKζ, or FLI-1 polypeptide in the cell;
a TRAC, B2M, and CIITA polypeptide in the cell;
a CD3g, B2M, and CIITA polypeptide in the cell; and
a PD1 polypeptide in the cell.

5. The method of claim 1, wherein the guide polynucleotide of (ii) is selected from the group consisting of EF8, EF1, EF2, EF3, EF4, EF5, EF6, EF7, EF9, EF10, EF11, EF12, EF13, EF14, EF15, EF16, EF17, EF18, EF19, EF20, EF21, EF22, EF23, EF24, EF25, EF26, EF27, EF28, EF29, EF46, EF47, EF48, EF49, EF50, EF51, EF52, EF53, EF54, EF55, EF56, EF57, EF58, EF59, EF60, EF61, EF62, EF63, EF64, EF65, EF66, EF67, EF68, EF69, EF70, EF71, EF72, EF73, EF74, EF75, EF76, EF77, EF85, EF86, EF87, EF88, EF89, EF90, EF91, EF92, EF93, EF94, EF95, EF96, EF97, EF98, EF99, EF100, EF101, EF102, EF103, EF104, EF105, EF106, EF107, EF108, EF109, EF110, EF111, EF112, EF113, EF114, EF115, EF116, EF117, EF118, EF119, and EF120.

6. A modified immune cell having reduced exhaustion after antigen exposure relative to an unedited immune effector cell, wherein the modified immune cell is produced according to the method of claim 1.

7. A modified immune cell having reduced exhaustion after antigen exposure relative to an unedited immune effector cell, the modified immune cell comprising a nucleobase alteration that reduces or eliminates expression of:
(i) a polypeptide selected from one or more of Roquin-1, ARID1A, BATF, CBLB, CD5, Chop, CISH, DCK, DGKa, DGK, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(ii) a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

8. The immune cell of claim 7, further comprising one or two chimeric antigen receptors targeting one or two antigens associated with a disease or disorder.

9. A modified immune effector cell having reduced exhaustion after antigen exposure relative to an unedited immune effector cell, wherein the modified immune effector cell expresses a chimeric antigen receptor targeting an antigen associated with a disease or disorder, and wherein the modified immune effector cell comprises reduced or undetectable expression of:
(i) one or more of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(ii) one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

10. The immune cell of claim 9, wherein the modified immune cell comprises reduced or undetectable expression of an antigen bound by the chimeric antigen receptor.

11. The immune cell of claim 9, wherein the modified immune cell comprises reduced or undetectable levels of expression of a polypeptide or a set or polypeptide selected from:
Roquin-1, DGKζ, or FLI-1 polypeptides;
TRAC, B2M, and CIITA polypeptides;
CD3g, B2M, and CIITA polypeptides; and
A PD1 polypeptide.

12. The immune cell of claim 9, wherein a population of the modified immune cells comprise:
an alteration in the ratio of CD4+ to CD8+ cells following one or more exposures to antigen relative to a population of unedited immune cells;
an increase in the proportion of CD4+ cells following one or more exposures to antigen relative to a population of unedited immune cells; an increase in CD25, ICOS, OX40, and/or CD28 expression following one or more exposures to antigen relative to an unedited immune cell.

13. A base editor system that comprises:
(i) a base editor, or a polynucleotide encoding the base editor, wherein the base editor comprises a programmable DNA binding domain and a deaminase domain;
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(iii) a guide polynucleotide, or a polynucleotide encoding the guide polynucleotide, wherein the guide polynucleotide directs the base editor to effect a nucleobase alteration in a polynucleotide encoding a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

14. The base editor system of claim 13, wherein the guide polynucleotide of (ii) comprises a polynucleotide sequence with at least about 85% sequence identity to a sequence selected from those listed in Table 1 or Table 2A, or a variant thereof comprising an extension or truncation 1, 2, 3, 4, or 5 nucleotides in length at the 3′ and/or 5′ end.

15. The base editor system of claim 13, wherein the guide polynucleotide of (ii) is selected from the group consisting of EF8, EF1, EF2, EF3, EF4, EF5, EF6, EF7, EF9, EF10, EF11, EF12, EF13, EF14, EF15, EF16, EF17, EF18, EF19, EF20, EF21, EF22, EF23, EF24, EF25, EF26, EF27, EF28, EF29, EF46, EF47, EF48, EF49, EF50, EF51, EF52, EF53, EF54, EF55, EF56, EF57, EF58, EF59, EF60, EF61, EF62, EF63, EF64, EF65, EF66, EF67, EF68, EF69, EF70, EF71, EF72, EF73, EF74, EF75, EF76, EF77, EF85, EF86, EF87, EF88, EF89, EF90, EF91, EF92, EF93, EF94, EF95, EF96, EF97, EF98, EF99, EF100, EF101, EF102, EF103, EF104, EF105, EF106, EF107, EF108, EF109, EF110, EF111, EF112, EF113, EF114, EF115, EF116, EF117, EF118, EF119, and EF120.

16. A cell comprising the base editor system of claim 13.

17. A vector or set of vectors comprising:
(i) a polynucleotide encoding a base editor, wherein the base editor comprises a programmable DNA binding domain and a deaminase domain;
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(iii) a guide polynucleotide, or a polynucleotide encoding the guide polynucleotide, wherein the guide polynucleotide directs the base editor to effect a nucleobase alteration in a polynucleotide encoding a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

18. A pharmaceutical composition comprising an effective amount of the modified immune cell of claim 9 and a pharmaceutically acceptable excipient.

19. A pharmaceutical composition comprising:
(i) a base editor, or a polynucleotide encoding the base editor, wherein the base editor comprises a programmable DNA binding domain and a deaminase domain;
(ii) a guide polynucleotide, or a polynucleotide encoding the guide polynucleotide, wherein the guide polynucleotide directs the base editor to effect a nucleobase alteration in a polynucleotide encoding a polypeptide selected from the group consisting of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(iii) a guide polynucleotide, or a polynucleotide encoding the guide polynucleotide, wherein the guide polynucleotide directs the base editor to effect a nucleobase alteration in a polynucleotide encoding a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC).

20. A method of treating a disease or disorder in a subject, the method comprising administering to the subject an effective amount of a modified immune cell, the cell, or the pharmaceutical composition of claim 19.

21. A kit comprising a modified immune effector cell having reduced exhaustion after antigen exposure relative to an unedited immune effector cell, wherein the modified immune effector cell expresses a chimeric antigen receptor targeting an antigen associated with a disease or disorder, and wherein the modified immune effector cell comprises reduced or undetectable expression of:
(i) one or more of Roquin-1, ARID1A, BATF, CBLB, CD5, Chop, CISH, DCK, DGKα, DGKζ, DHX37, DNMT3A, EIF2A, FLI-1, ID3, IKZF2, IL-6, PFN1, PRDM1, PRKACA, PTP1B, PTPN6, RASA2, Regnase-1, SOCS1, SOX4, TLE, TMEM184B, and TMEM222; and
(ii) one or more of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC)
for use in the method of claim 20.

22. A method for producing a modified immune effector cell having reduced exhaustion after antigen exposure relative to an unedited immune effector cell, the method comprising contacting the cell with:
(i) a base editor, or a polynucleotide encoding the base editor, wherein the base editor comprises a programmable DNA binding domain and a deaminase domain;
(ii) a guide polynucleotide, or a polynucleotide encoding the guide polynucleotide, wherein the guide polynucleotide directs the base editor to effect a nucleobase alteration in a polynucleotide encoding a Roquin-1 polypeptide; and
(iii) a guide polynucleotide, or a polynucleotide encoding the guide polynucleotide, wherein the guide polynucleotide directs the base editor to effect a nucleobase alteration in a polynucleotide encoding a polypeptide selected from the group consisting of beta-2-microglobulin (B2M), cluster of differentiation 3-epsilon (CD3e), cluster of differentiation 3-gamma (CD3g), class II major histocompatibility complex transactivator (CIITA), programmed cell death 1 (PD1), and T cell receptor constant region (TRAC);
wherein each nucleobase alteration effects a reduction in expression of the encoded polypeptide, thereby producing the modified immune effector cell.

23. A method for reducing or eliminating the presence of a neoplasia in a subject, the method comprising administering to the subject a cell produced according to the method of claim 22, wherein the cell expresses a chimeric antigen receptor targeting an antigen associated with the neoplasia.
